SEQUENCE LISTING (5 sequences)

SEQ ID NO: 1 - protein, 1296 aa, Clostridium botulinum serotype A [sequence omitted]

SEQ ID NO: 2 - protein, 1296 aa, Clostridium botulinum serotype A
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHDVLNLTRNGYGSTQYIRFSPDFTFGFEESLEVDTNPLLGAGKFATDPAVTLAHELIHAEHRLYGIAINPNRVFKVNTNAYYEMSGLEVSFEELRTFGGHDAKFIDSLQENEFRLYYYNKFKDVASTLNKAKSIIGTTASLQYMKNVFKEKYLLSEDTSGKFSVDKLKFDKLYKMLTEIYTEDNFVNFFKVINRKTYLNFDKAVFRINIVPDENYTIKDGFNLKGANLSTNFNGQNTEINSRNFTRLKNFTGLFEFYKLLCVRGIIPFKTKSLDEGYNKALNDLCIKVNNWDLFFSPSEDNFTNDLDKVEEITADTNIEAAEENISLDLIQQYYLTFDFDNEPENISIENLSSDIIGQLEPMPNIERFPNGKKYELDKYTMFHYLRAQEFEHGDSRIILTNSAEEALLKPNVAYTFFSSKYVKKINKAVEAFMFLNWAEELVYDFTDETNEVTTMDKIADITIIVPYIGPALNIGNMLSKGEFVEAIIFTGVVAMLEFIPEYALPVFGTFAIVSYIANKVLTVQTINNALSKRNEKWDEVYKYTVTNWLAKVNTQIDLIREKMKKALENQAEATKAIINYQYNQYTEEEKNNINFNIDDLSSKLNESINSAMININKFLDQCSVSYLMNSMIPYAVKRLKDFDASVRDVLLKYIYDNRGTLVLQVDRLKDEVNNTLSADIPFQLSKYVDNKKLLSTFTEYIKNIVNTSILSIVYKKDDLIDLSRYGAKINIGDRVYYDSIDKNQIKLINLESSTIEVILKNAIVYNSMYENFSTSFWIKIPKYFSKINLNNEYTIINCIENNSGWKVSLNYGEIIWTLQDNKQNIQRVVFKYSQMVNISDYINRWIFVTITNNRLTKSKIYINGRLIDQKPISNLGNIHASNKIMFKLDGCRDPRRYIMIKYFNLFDKELNEKEIKDLYDSQSNSGILKDFWGNYLQYDKPYYMLNLFDPNKYVDVNNIGIRGYMYLKGPRGSVVTTNIYLNSTLYEGTKFIIKKYASGNEDNIVRNNDRVYINVVVKNKEYRLATNASQAGVEKILSALEIPDVGNLSQVVVMKSKDDQGIRNKCKMNLQDNNGNDIGFIGFHLYDNIAKLVASNWYNRQVGKASRTFGCSWEFIPVDDGWGESSL

SEQ ID NO: 3 - protein, 1292 aa, Clostridium botulinum serotype A
MPFVNKPFNYRDPGNGVDIAYIKIPNAGQMQPVKAFKIHEGVWVIPERDTFTNPEEGDLNPPPEAKQVPVSYYDSTYLSTDNEKDNYLKGVIKLFDRIYSTGLGRMLLSFIVKGIPFWGGSTIDTELKVIDTNCINVIEPGGSYRSEELNLVITGPSADIIQFECKSFGHDVFNLTRNGYGSTQYIRFSPDFTFGFEESLEVDTNPLLGAGTFATDPAVTLAHELIHAAHRLYGIAINPNRVLKVKTNAYYEMSGLEVSFEELRTFGGNDTNFIDSLWQKKFSRDAYDNLQNIARILNEAKTIVGTTTPLQYMKNIFIRKYFLSEDASGKISVNKAAFKEFYRVLTRGFTELEFVNPFKVINRKTYLNFDKAVFRINIVPDENYTINEGFNLEGANSNGQNTEINSRNFTRLKNFTGLFEFYKLLCVRGIIPFKTKSLDEGYNKALNYLCIKVNNWDLFFSPSEDNFTNDLDKVEEITADTNIEAAEENISSDLIQQYYLTFDFDNEPENISIENLSSDIIGQLEPMPNIERFPNGKKYELDKYTMFHYLRAQEFEHGDSRIILTNSAEEALLKPNVAYTFFSSKYVKKINKAVEAVIFLSWAEELVYDFTDETNEVTTMDKIADITIIVPYIGPALNIGNMVSKGEFVEAILFTGVVALLEFIPEYSLPVFGTFAIVSYIANKVLTVQTINNALSKRNEKWDEVYKYTVTNWLAKVNTQIDLIREKMKKALENQAEATRAIINYQYNQYTEEEKNNINFNIDDLSSKLNRSINRAMININKFLDQCSVSYLMNSMIPYAVKRLKDFDASVRDVLLKYIYDNRGTLILQVDRLKDEVNNTLSADIPFQLSKYVNDKKLLSTFTEYIKNIVNTSILSIVYKKDDLIDLSRYGAKINIGDRVYYDSIDKNQIKLINLESSTIEVILKNAIVYNSMYENFSTSFWIKIPKYFSKINLNNEYTIINCIENNSGWKVSLNYGEIIWTLQDNKQNIQRVVFKYSQMVNISDYINRWMFVTITNNRLTKSKIYINGRLIDQKPISNLGNIHASNKIMFKLDGCRDPRRYIMIKYFNLFDKELNEKEIKDLYDSQSNPGILKDFWGNYLQYDKPYYMLNLFDPNKYVDVNNIGIRGYMYLKGPRGSVMTTNIYLNSTLYMGTKFIIKKYASGNEDNIVRNNDRVYINVVVKNKEYRLATNASQAGVEKILSALEIPDVGNLSQVVVMKSKDDQGIRNKCKMNLQDNNGNDIGFVGFHLYDNIAKLVASNWYNRQVGKASRTFGCSWEFIPVDDGWGESSL

SEQ ID NO: 4 - protein, 1296 aa, Clostridium botulinum serotype A
MPLVNQQINYYDPVNGVDIAYIKIPNAGKMQPVKAFKIHNKVWVIPERDIFTNPEEVDLNPPPEAKQVPISYYDSAYLSTDNEKDNYLKGVIKLFERIYSTDLGRMLLISIVRGIPFWGGGKIDTELKVIDTNCINIIQLDDSYRSEELNLAIIGPSANIIESQCSSFRDDVLNLTRNGYGSTQYIRFSPDFTVGFEESLEVDTNPLLGAGKFAQDPAVALAHELIHAEHRLYGIAINTNRVFKVNTNAYYEMAGLEVSLEELITFGGNDAKFIDSLQKKEFSLYYYNKFKDIASTLNKAKSIVGTTASLQYMKNVFKEKYLLSEDATGKFLVDRLKFDELYKLLTEIYTEDNFVKFFKVLNRKTYLNFDKAVFKINIVPDVNYTIHDGFNLRNTNLAANFNGQNIEINNKNFDKLKNFTGLFEFYKLLCVRGIITSKTKSLDEGYNKALNELCIKVNNWDLFFSPSEDNFTNDLDKVEEITSDTNIEAAEENISLDLIQQYYLNFNFDNEPENTSIENLSSDIIGQLEPMPNIERFPNGKKYELNKYTMFHYLRAQEFKHSNSRIILTNSAKEALLKPNIVYTFFSSKYIKAINKAVEAVTFVNWIENLVYDFTDETNEVSTMDKIADITIVIPYIGPALNIGNMIYKGEFVEAIIFSGAVILLEIVPEIALPVLGTFALVSYVSNKVLTVQTIDNALSKRNEKWDEVYKYIVTNWLAIVNTQINLIREKMKKALENQAEATKAIINYQYNQYTEEEKNNINFNIDDLSSKLNESINSAMININKFLDQCSVSYLMNSMIPYAVKRLKDFDASVRDVLLKYIYDNRGTLIGQVNRLKDKVNNTLSADIPFQLSKYVDNKKLLSTFTEYIKNITNASILSIVYKDDDLIDLSRYGAEIYNGDKVYYNSIDKNQIRLINLESSTIEVILKKAIVYNSMYENFSTSFWIRIPKYFNSISLNNEYTIINCMENNSGWKVSLNYGEIIWTFQDTQEIKQRVVFKYSQMINISDYINRWIFVTITNNRITKSKIYINGRLIDQKPISNLGNIHASNKIMFKLDGCRDPHRYIVIKYFNLFDKELSEKEIKDLYDNQSNSGILKDFWGDYLQYDKSYYMLNLYDPNKYVDVNNVGIRGYMYLKGPRDNVMTTNIYLNSSLYMGTKFIIKKYASGNKDNIVRNNDRVYINVVVKNKEYRLATNASQAGVEKILSALEIPDVGNLSQVVVMKSKNDQGITNKCKMNLQDNNGNDIGFIGFHQFNNIAKLVASNWYNRQIERSSRTLGCSWEFIPVDDGWRERPL

SEQ ID NO: 5 - protein, 57 aa, Clostridium botulinum serotype E
KNELTNKYDIKQIENELNQKVSIAMNNIDRFLTESSISYLMKLINEVKINKLREYDE

(19) United States
(12) Patent Application Publication
(10) Pub. No.: US 2007/0258992 A1
(43) Pub. Date: Nov. 8, 2007

(54) Determining and Reducing Immunoresistance to Botulinum Toxin Therapy Using Botulinum Toxin a Peptides

(76) Inventor: M. Zouhair Atassi, Houston, TX (US)

Correspondence Address: ALLERGAN, INC., 2525 DUPONT DRIVE, T2-7H, IRVINE, CA, 92612-1599, US

(21) Appl. No.: 11/576,219

(22) PCT Filed: Oct. 5, 2005
(86) PCT No.: PCT/US05/36229

§ 371 Date: Mar. 28, 2007

Related U.S. Application Data
(60) Provisional application No. 60/616,682, filed on Oct. 6, 2004.

(51) Int. Cl. A61K 39/08; A61P 21/00; C07K 14/33; C12P 21/00; G01N 33/48; G01N 33/536

(52) U.S. Cl. 424/190.1; 435/7.92; 435/70.3; 436/501; 436/513; 530/324; 530/326

(57) ABSTRACT

The present invention provides BoNT/A peptide compositions, tolerogizing compositions, vaccine compositions and antibody compositions, as well as methods of determining immunoresistance to botulinum toxin therapy in an individual, methods of preventing or reducing immunoresistance to botulinum toxin therapy in an individual, methods of vaccinating an individual against botulinum toxin, methods of preparing anti-BoNT/A antibodies, methods of treating botulinum toxicity in an individual and methods of reducing anti-botulinum toxin antibodies in an individual.

### FIELD OF INVENTION

This invention relates generally to the field of immunology, and, more specifically, to the use of botulinum toxin peptides and anti-botulinum toxin antibodies as diagnostic and therapeutic agents.

### BACKGROUND OF THE INVENTION

Botulinum neurotoxins are proteins produced by several strains of the bacterium Clostridium botulinum, the spores of which are abundant in soil and marine sediments. These proteins are the most toxic substances known to man, being more lethal per molecule than diphtheria toxin, curare and sodium cyanide. There are seven distinct but related botulinum toxin serotypes, designated A through G. Botulinum toxin types A, B, E, and F are the most common causes of botulism in humans, while types C and D cause botulism in other mammals and birds. All seven botulinum toxin serotypes act by similar mechanisms and produce similar lethal effects when inhaled or ingested.

Botulinum toxins interrupt signals normally transmitted from nerve to muscle, thereby resulting in paralysis. Normally, electrical impulses that control muscle function are generated by the brain, brain stem and spinal cord, and these impulses travel from the originating area into peripheral nerves, which control motor function. At the end of these peripheral nerves are compartments for the neurotransmitter acetylcholine, a chemical messenger that transmits the electrical signal of the peripheral nerve to the muscle, instructing the muscle to contract. In the absence of botulinum toxin, acetylcholine is released into the junction between peripheral nerve and muscle when an electrical impulse reaches the storage compartment. The released acetylcholine binds to receptors located on the muscle, signaling the ensuing muscle contraction. However, botulinum toxin interferes with the release of acetylcholine into the junction, thereby blocking transmission of the electrical signal. Normal muscular contraction terminates due to the absence of the electrical signal.

The myorelaxant properties of BoNTs are being exploited in a wide variety of therapeutic and cosmetic applications, see, e.g., William J. Lipham, COSMETIC AND CLINICAL APPLICATIONS OF BOTULINUM TOXIN (Slack, Inc., 2004). For example, BoNT/A has emerged as an important therapeutic treatment for a number of neurological and ophthalmic disorders that have few other effective remedies, such as, e.g., cervical dystonia (asymmetric muscular spasm in the neck that results in forceful turning of the head), strabismus (misalignment of the eyes), focal spasm, such as, e.g., hemifacial spasm (sudden unilateral muscle contractions of the face), and blepharospasm (forceful involuntary closure of the eyelids). Subsequently, proposed uses of BoNT/A as a biopharmaceutical neuromodulator has expanded to cover a wide variety of disorders where chemodenervation of the neuromuscular junctions may be beneficial, such as, e.g., without limitation, chronic lower back pain, oromandibular dystonia (continuous spasms of the face, jaw, neck, tongue, larynx, and respiratory system), spasmodic dysphonia (spasm of the vocal cords that causes sudden disruption of speech), stuttering and voice tremors, and various focal and segmental dystonias. In addition, BoNT/A treatments targeting certain disorders that lack a neuromuscular basis were developed. For example, the effects on the autonomic nervous system that BoNT/A may be of use in treating axillary hyperhydrosis or sweating, and reports indicate BoNT/A may be an effective treatment for myofascial pain and tension, stroke, traumatic brain injury, cerebral palsy, gastrointestinal motility disorders, urinary incontinence cancer and migraine headaches. Lastly, cosmetic and other therapeutic applications are widely known. In fact, the expected use of BoNT/A in both therapeutic and cosmetic treatments of humans is anticipated to expand to an ever widening range of diseases and aliments that can benefit from the myorelaxant properties of this toxin.

While a potent and effective treatment, the inhibition of neurotransmitter release and the resulting neuromuscular paralysis elicited by BoNT/A is not permanent. The reversible nature of these paralytic effects requires periodic treatments in order to maintain the therapeutic benefits from this toxin. As a consequence of this repeated exposure, an immune response against BoNT/A can occur in some patients which reduce or completely prevent the individual's responsiveness to further treatments, see, e.g., Joseph Jankovic, Botulinum toxin: Clinical Implications of Antigenicity and Immunoresistance, (SCIENTIFIC AND THERAPEUTIC ASPECTS OF BOTULINUM TOXIN, 409-415, Mitchell F. Brin et al., eds., Lippincott Williams & Wilkins, 2002); Dirk Dressler, Clinical Presentation and Management of Antibody-induced Failure of Botulinum Toxin Therapy, 19(Suppl. 8) Mov. DISORD. S92-S100 (2004); M. Zouhair Atassi, Basic Immunological Aspects of Botulinum Toxin Therapy, 19(Suppl. 8) MOV. DISORD. S68-S84, (2004).

Thus, there exists a need for methods of predicting or determining immunoresistance in an individual to botulinum toxin therapy, methods of preventing or reducing immunoresistance in an individual to botulinum toxin therapy as well as compositions to carry out these methods. The present invention satisfies this need and provides related advantages as well.

### SUMMARY OF INVENTION

The present invention provides a BoNT/A peptide composition having a length of at most 60 amino acids and consisting of at least 5 contiguous amino acids selected from one of the following BoNT/A amino acid sequences: 449-467 of SEQ ID NO:1(N1), 463-481 of SEQ ID NO:1 (N2), 491-509 of SEQ ID NO:1 (N4), 505-523 of SEQ ID NO:1 (N5), 519-537 of SEQ ID NO:1 (N6), 533-551 of SEQ ID NO:1 (N7), 547-565 of SEQ ID NO:1 (N8), 561-579 of SEQ ID NO:1 (N9), 575-593 of SEQ ID NO:1 (N10), 589-607 of SEQ ID NO:1 (N11), 631-649 of SEQ ID NO:1 (N14), 645-663 of SEQ ID NO:1 (N15), 659-677 of SEQ ID NO:1 (N16), 673-691 of SEQ ID NO:1 (N17), 701-719 of SEQ ID NO:1 (N19), 715-733 of SEQ ID NO:1 (N20), 729-747 of SEQ ID NO:1 (N21), 743-761 of SEQ ID NO:1 (N22), 757-775 of SEQ ID NO:1 (N23), 771-789 of SEQ ID NO:1 (N24), 785-803 of SEQ ID NO:1 (N25), 799-817 of SEQ ID NO:1 (N26), 813-831 of SEQ ID NO:1 (N27), 827-845 of SEQ ID NO:1 (N28), 869-887 of SEQ ID NO:1 (C2), 883-901 of SEQ ID NO:l (C3), 911-929 of SEQ ID NO:l (C5), 925-943 of SEQ ID NO:1 (C6), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1065-1083 of SEQ ID NO:1 (C16), 1079-1097 of SEQ ID NO:1 (C17), 1107-1125 of SEQ ID NO:1 (C19), 1121-1139 of SEQ ID NO:1 (C20), 1135-1153 of SEQ ID NO:1 (C21), 1149-1167 of SEQ ID NO:1 (C22), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28), 1247-1265 of SEQ ID NO:1 (C29), 1261-1279 of SEQ ID NO:1 (C30), or 1275-1296 of SEQ ID NO:1 (C31), or a conservative variant, or a non-conservative variant, or immunoreactive fragment thereof, with the proviso that the BoNT/A peptide is not SEQ ID NO:2.

The present invention further provides a tolerogizing composition comprising a tolerogizing agent and a BoNT/A peptide having a length of at most 60 amino acids and consisting of at least 5 contiguous amino acids selected from one of the following BoNT/A amino acid sequences: 449-467 of SEQ ID NO:1 (N1), 463-481 of SEQ ID NO:1 (N2), 491-509 of SEQ ID NO:1 (N4), 505-523 of SEQ ID NO:1 (N5), 519-537 of SEQ ID NO:1 (N6), 533-551 of SEQ ID NO:1 (N7), 547-565 of SEQ ID NO:1 (N8), 561-579 of SEQ ID NO:1 (N9), 575-593 of SEQ ID NO:1 (N10), 589-607 of SEQ ID NO:1 (N11), 631-649 of SEQ ID NO:1 (N14), 645-663 of SEQ ID NO:1 (N15), 659-677 of SEQ ID NO:1 (N16), 673-691 of SEQ ID NO:1 (N17), 701-719 of SEQ ID NO:1 (N19), 715-733 of SEQ ID NO:1 (N20), 729-747 of SEQ ID NO:1 (N21), 743-761 of SEQ ID NO:1 (N22), 757-775 of SEQ ID NO:1 (N23), 771-789 of SEQ ID NO:1 (N24), 785-803 of SEQ ID NO:1 (N25), 799-817 of SEQ ID NO:1 (N26), 813-831 of SEQ ID NO:1 (N27), 827-845 of SEQ ID NO:1 (N28), 869-887 of SEQ ID NO:1 (C2), 883-901 of SEQ ID NO:1 (C3), 911-929 of SEQ ID NO:1 (C5), 925-943 of SEQ ID NO:1 (C6), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1065-1083 of SEQ ID NO:1 (C16), 1079-1097 of SEQ ID NO:1 (C17), 1107-1125 of SEQ ID NO:1 (C19), 1121-1139 of SEQ ID NO:1 (C20), 1135-1153 of SEQ ID NO:1 (C21), 1149-1167 of SEQ ID NO:1 (C22), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28), 1247-1265 of SEQ ID NO:1 (C29), 1261-1279 of SEQ ID NO:l (C30), or 1275-1296 of SEQ ID NO:1 (C31), or a conservative variant or tolerogenic fragment thereof, with the proviso that the BoNT/A peptide is not SEQ ID NO:2.

The present invention further provides a vaccine composition comprising a BoNT/A peptide having a length of at most 60 amino acids and consisting of at least 5 contiguous amino acids selected from one of the following BoNT/A amino acid sequences: 449-467 of SEQ ID NO:1(N1), 463-481 of SEQ ID NO:1 (N2), 491-509 of SEQ ID NO:1 (N4), 505-523 of SEQ ID NO:1 (N5), 519-537 of SEQ ID NO:1 (N6), 533-551 of SEQ ID NO:1 (N7), 547-565 of SEQ ID NO:1 (N8), 561-579 of SEQ ID NO:1 (N9), 575-593 of SEQ ID NO:1 (N10), 589-607 of SEQ ID NO:1 (N11), 631-649 of SEQ ID NO:1 (N14), 645-663 of SEQ ID NO:1 (N15), 659-677 of SEQ ID NO:1 (N16), 673-691 of SEQ ID NO:1 (N17), 701-719 of SEQ ID NO:1 (N19), 715-733 of SEQ ID NO:1 (N20), 729-747 of SEQ ID NO:1 (N21), 743-761 of SEQ ID NO:1 (N22), 757-775 of SEQ ID NO:1 (N23), 771-789 of SEQ ID NO:1 (N24), 785-803 of SEQ ID NO:1 (N25), 799-817 of SEQ ID NO:1 (N26), 813-831 of SEQ ID NO:1 (N27), 827-845 of SEQ ID NO:1 (N28), 869-887 of SEQ ID NO:1 (C2), 883-901 of SEQ ID NO:1 (C3), 911-929 of SEQ ID NO:1 (C5), 925-943 of SEQ ID NO:1 (C6), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1065-1083 of SEQ ID NO:1 (C16), 1079-1097 of SEQ ID NO:1 (C17), 1107-1125 of SEQ ID NO:1 (C19), 1121-1139 of SEQ ID NO:1 (C20), 1135-1153 of SEQ ID NO:1 (C21), 1149-1167 of SEQ ID NO:1 (C22), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28), 1247-1265 of SEQ ID NO:1 (C29), 1261-1279 of SEQ ID NO:1 (C30), or 1275-1296 of SEQ ID NO:1 (C31), or a conservative variant or immunoreactive fragment thereof, with the proviso that the BoNT/A peptide is not SEQ ID NO:2.

The present invention further provides an antibody composition produced from a BoNT/A peptide having a length of at most 60 amino acids and consisting of at least 5 contiguous amino acids selected from one of the following BoNT/A amino acid sequences: 449-467 of SEQ ID NO:1(N1), 463-481 of SEQ ID NO:1 (N2), 491-509 of SEQ ID NO:1 (N4), 505-523 of SEQ ID NO:1 (N5), 519-537 of SEQ ID NO:1 (N6), 533-551 of SEQ ID NO:1 (N7), 547-565 of SEQ ID NO:1 (N8), 561-579 of SEQ ID NO:1 (N9), 575-593 of SEQ ID NO:1 (N10), 589-607 of SEQ ID NO:1 (N11), 631-649 of SEQ ID NO:1 (N14), 645-663 of SEQ ID NO:1 (N15), 659-677 of SEQ ID NO:1 (N16), 673-691 of SEQ ID NO:1 (N17), 701-719 of SEQ ID NO:1 (N19), 715-733 of SEQ ID NO:1 (N20), 729-747 of SEQ ID NO:1 (N21), 743-761 of SEQ ID NO:1 (N22), 757-775 of SEQ ID NO:1 (N23), 771-789 of SEQ ID NO:1 (N24), 785-803 of SEQ ID NO:1 (N25), 799-817 of SEQ ID NO:1 (N26), 813-831 of SEQ ID NO:1 (N27), 827-845 of SEQ ID NO:1 (N28), 869-887 of SEQ ID NO:1 (C2), 883-901 of SEQ ID NO:1 (C3), 911-929 of SEQ ID NO:1 (C5), 925-943 of SEQ ID NO:1 (C6), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1065-1083 of SEQ ID NO:1 (C16), 1079-1097 of SEQ ID NO:1 (C17), 1107-1125 of SEQ ID NO:1 (C19), 1121-1139 of SEQ ID NO:1 (C20), 1135-1153 of SEQ ID NO:1 (C21), 1149-1167 of SEQ ID NO:1 (C22), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28), 1247-1265 of SEQ ID NO:1 (C29), 1261-1279 of SEQ ID NO:1 (C30), or 1275-1296 of SEQ ID NO:1 (C31), or a conservative variant or immunoreactive fragment thereof, with the proviso that the BoNT/A peptide is not SEQ ID NO:2.

The present invention also provides a method of determining immunoresistance to botulinum toxin therapy in a human or other mammal by determining the presence or absence in said human or other mammal of antibodies immunoreactive with a BoNT/A peptide having a length of at most 60 amino acids and consisting of at least 5 contiguous amino acids selected from one of the following BoNT/A amino acid sequences: 449-467 of SEQ ID NO:1(N1), 463-481 of SEQ ID NO:1 (N2), 491-509 of SEQ ID NO:1 (N4), 505-523 of SEQ ID NO:1 (N5), 519-537 of SEQ ID NO:1 (N6), 533-551 of SEQ ID NO:1 (N7), 547-565 of SEQ ID NO:1 (N8), 561-579 of SEQ ID NO:1 (N9), 575-593 of SEQ ID NO:1 (N10), 589-607 of SEQ ID NO:1 (N11), 631-649 of SEQ ID NO:1 (N14), 645-663 of SEQ ID NO:1 (N15), 659-677 of SEQ ID NO:1 (N16), 673-691 of SEQ ID NO:1 (N17), 701-719 of SEQ ID NO:1 (N19), 715-733 of SEQ ID NO:1 (N20), 729-747 of SEQ ID NO:1 (N21), 743-761 of SEQ ID NO:1 (N22), 757-775 of SEQ ID NO:1 (N23), 771-789 of SEQ ID NO:1 (N24), 785-803 of SEQ ID NO:1 (N25), 799-817 of SEQ ID NO:1 (N26), 813-831 of SEQ ID NO:1 (N27), 827-845 of SEQ ID NO:1 (N28), 869-887 of SEQ ID NO:1 (C2), 883-901 of SEQ ID NO:1 (C3), 911-929 of SEQ ID NO:1 (C5), 925-943 of SEQ ID NO:1 (C6), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1065-1083 of SEQ ID NO:1 (C16), 1079-1097 of SEQ ID NO:1 (C17), 1107-1125 of SEQ ID NO:1 (C19), 1121-1139 of SEQ ID NO:1 (C20), 1135-1153 of SEQ ID NO:1 (C21), 1149-1167 of SEQ ID NO:1 (C22), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28), 1247-1265 of SEQ ID NO:1 (C29), 1261-1279 of SEQ ID NO:1 (C30), or 1275-1296 of SEQ ID NO:1 (C31), or a conservative variant, or a non-conservative variant, or immunoreactive fragment thereof, where the presence of antibodies immunoreactive with the peptide indicates immunoresistance to botulinum toxin therapy, with the proviso that the BoNT/A peptide is not SEQ ID NO:2.

The present invention also provides a method of preventing or reducing immunoresistance to botulinum toxin therapy in a human or other mammal by administering to said human or other mammal a tolerogizing composition comprising a tolerogizing agent and a BoNT/A peptide having a length of at most 60 amino acids and consisting of at least 5 contiguous amino acids selected from one of the following BoNT/A amino acid sequences: 449-467 of SEQ ID NO:1(N1), 463-481 of SEQ ID NO:1 (N2), 491-509 of SEQ ID NO:1 (N4), 505-523 of SEQ ID NO:1 (N5), 519-537 of SEQ ID NO:1 (N6), 533-551 of SEQ ID NO:1 (N7), 547-565 of SEQ ID NO:1 (N8), 561-579 of SEQ ID NO:1 (N9), 575-593 of SEQ ID NO:1 (N10), 589-607 of SEQ ID NO:1 (N11), 631-649 of SEQ ID NO:1 (N14), 645-663 of SEQ ID NO:l (N15), 659-677 of SEQ ID NO:1 (N16), 673-691 of SEQ ID NO:1 (N17), 701-719 of SEQ ID NO:1 (N19), 715-733 of SEQ ID NO:1 (N20), 729-747 of SEQ ID NO:1 (N21), 743-761 of SEQ ID NO:1 (N22), 757-775 of SEQ ID NO:1 (N23), 771-789 of SEQ ID NO:1 (N24), 785-803 of SEQ ID NO:1 (N25), 799-817 of SEQ ID NO:1 (N26), 813-831 of SEQ ID NO:1 (N27), 827-845 of SEQ ID NO:1 (N28), 869-887 of SEQ ID NO:1 (C2), 883-901 of SEQ ID NO:1 (C3), 911-929 of SEQ ID NO:1 (C5), 925-943 of SEQ ID NO:1 (C6), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1065-1083 of SEQ ID NO:1 (C16), 1079-1097 of SEQ ID NO:1 (C17), 1107-1125 of SEQ ID NO:1 (C19), 1121-1139 of SEQ ID NO:1 (C20), 1135-1153 of SEQ ID NO:1 (C21), 1149-1167 of SEQ ID NO:1 (C22), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28), 1247-1265 of SEQ ID NO:1 (C29), 1261-1279 of SEQ ID NO:1 (C30), or 1275-1296 of SEQ ID NO:1 (C31), or a conservative variant or tolerogenic fragment thereof, thereby preventing or reducing immunoresistance to botulinum toxin therapy, with the proviso that the BoNT/A peptide is not SEQ ID NO:2.

The present invention also provides a method of vaccinating a human or other mammal against botulinum toxin by administering to said human or other mammal a vaccine composition comprising a BoNT/A peptide having a length of at most 60 amino acids and consisting of at least 5 contiguous amino acids selected from one of the following BoNT/A amino acid sequences: 449-467 of SEQ ID NO:1(N1), 463-481 of SEQ ID NO:1 (N2), 491-509 of SEQ ID NO:1 (N4), 505-523 of SEQ ID NO:1 (N5), 519-537 of SEQ ID NO:1 (N6), 533-551 of SEQ ID NO:1 (N7), 547-565 of SEQ ID NO:1 (N8), 561-579 of SEQ ID NO:1 (N9), 575-593 of SEQ ID NO:1 (N10), 589-607 of SEQ ID NO:1 (N11), 631-649 of SEQ ID NO:1 (N14), 645-663 of SEQ ID NO:1 (N15), 659-677 of SEQ ID NO:1 (N16), 673-691 of SEQ ID NO:1 (N17), 701-719 of SEQ ID NO:1 (N19), 715-733 of SEQ ID NO:1 (N20), 729-747 of SEQ ID NO:1 (N21), 743-761 of SEQ ID NO:1 (N22), 757-775 of SEQ ID NO:1 (N23), 771-789 of SEQ ID NO:1 (N24), 785-803 of SEQ ID NO:1 (N25), 799-817 of SEQ ID NO:1 (N26), 813-831 of SEQ ID NO:1 (N27), 827-845 of SEQ ID NO:1 (N28), 869-887 of SEQ ID NO:1 (C2), 883-901 of SEQ ID NO:1 (C3), 911-929 of SEQ ID NO:1 (C5), 925-943 of SEQ ID NO:1 (C6), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15),1065-1083 of SEQ ID NO:1 (C16), 1079-1097 of SEQ ID NO:1 (C17), 1107-1125 of SEQ ID NO:1 (C19), 1121-1139 of SEQ ID NO:1 (C20), 1135-1153 of SEQ ID NO:1 (C21), 1149-1167 of SEQ ID NO:1 (C22), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28), 1247-1265 of SEQ ID NO:1 (C29), 1261-1279 of SEQ ID NO:1 (C30), or 1275-1296 of SEQ ID NO:1 (C31), or a conservative variant or immunoreactive fragment thereof, thereby producing an immune response to botulinum toxin in said human or other mammal, with the proviso that the BoNT/A peptide is not SEQ ID NO:2.

The invention additionally provides a method of preparing an anti-BoNT/A antibody by administering to a human or other mammal a BoNT/A peptide having a length of at most 60 amino acids and consisting of at least 5 contiguous amino acids selected from one of the following BoNT/A amino acid sequences: 449-467 of SEQ ID NO:1(N1), 463-481 of SEQ ID NO:1 (N2), 491-509 of SEQ ID NO:1 (N4), 505-523 of SEQ ID NO:1 (N5), 519-537 of SEQ ID NO:1 (N6), 533-551 of SEQ ID NO:1 (N7), 547-565 of SEQ ID NO:1 (N8), 561-579 of SEQ ID NO:1 (N9), 575-593 of SEQ ID NO:1 (N10), 589-607 of SEQ ID NO:1 (N11), 631-649 of SEQ ID NO:1 (N14), 645-663 of SEQ ID NO:1 (N15), 659-677 of SEQ ID NO:1 (N16), 673-691 of SEQ ID NO:1 (N17), 701-719 of SEQ ID NO:1 (N19), 715-733 of SEQ ID NO:1 (N20), 729-747 of SEQ ID NO:1 (N21), 743-761 of SEQ ID NO:1 (N22), 757-775 of SEQ ID NO:1 (N23), 771-789 of SEQ ID NO:1 (N24), 785-803 of SEQ ID NO:1 (N25), 799-817 of SEQ ID NO:l (N26), 813-831 of SEQ ID NO:1 (N27), 827-845 of SEQ ID NO:1 (N28), 869-887 of SEQ ID NO:1 (C2), 883-901 of SEQ ID NO:1 (C3), 911-929 of SEQ ID NO:1 (C5), 925-943 of SEQ ID NO:1 (C6), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1065-1083 of SEQ ID NO:1 (C16), 1079-1097 of SEQ ID NO:1 (C17), 1107-1125 of SEQ ID NO:1 (C19), 1121-1139 of SEQ ID NO:1 (C20), 1135-1153 of SEQ ID NO:1 (C21), 1149-1167 of SEQ ID NO:l (C22), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28), 1247-1265 of SEQ ID NO:1 (C29), 1261-1279 of SEQ ID NO:1 (C30), or 1275-1296 of SEQ ID NO:1 (C31), or a conservative variant or immunoreactive fragment thereof; collecting from the animal a sample containing an antibody or antibody-producing cell; and processing the sample to isolate the anti-BoNT/A antibody, with the proviso that the BoNT/A peptide is not SEQ ID NO:2.

The present invention additionally provides a method of treating botulinum toxicity in a human or other mammal by administering to said human or other mammal a pharmaceutical composition comprising an anti-BoNT/A antibody composition disclosed in the present specification.

The present invention additionally provides a method of reducing botulinum toxin antibodies from a human or other mammal by removing blood from said human or other mammal; contacting the blood, or an antibody-containing component thereof, with a BoNT/A peptide disclosed in the present invention under conditions suitable for forming a complex of each of the amino acid sequences and anti-botulinum toxin antibody; and removing the complex from the blood or antibody-containing component thereof.

### BRIEF DESCRIPTION OF THE DRAWINGS

FIG. 1A shows synthetic consecutive overlapping peptides of the H_{N} domain of BoNT/A having the indicated residues of SEQ ID NO:1. Regions of overlap with adjacent peptides are underlined and bolded. FIG. 1B shows synthetic consecutive overlapping peptides of the H_{C} domain of BoNT/A having the indicated residues of SEQ ID NO:1. Regions of overlap with adjacent peptides are underlined and bolded. The L-peptide control sequence is shown as SEQ ID NO:1.

FIG. 2 shows binding of human anti-pentavalent botulinum toxoid antibodies to overlapping synthetic peptides spanning the BoNT/A H_{N} domain and to active H_{C} peptides. Also shown are binding to L-Peptide and full-length BoNT/A as negative and positive controls, respectively.

FIG. 3 shows binding of anti-pentavalent botulinum toxoid antibodies of ICR outbred mice to synthetic overlapping peptides spanning the BoNT/A H_{N} domain. Also shown are binding to L-Peptide and full-length BoNT/A as negative and positive controls, respectively.

FIG. 4 shows binding of chicken anti-BoNT/A antibodies to 60 synthetic overlapping peptides spanning the entire H-subunit of BoNT/A. Also shown are binding to L-Peptide and full-length BoNT/A as negative and positive controls, respectively.

FIG. 5 shows binding of horse anti-BoNT/A antibodies to active BoNT/A overlapping synthetic peptides spanning the BoNT/A H_{N} domain and to active H_{C} peptides. Also shown are binding to L-Peptide and full-length BoNT/A as negative and positive controls, respectively.

FIG. 6 shows amino acid sequences of the H_{N} domain of BoNT/A (SEQ ID NO:1); the Hc domain of BoNT/A (SEQ ID NO:1); the L peptide (SEQ ID NO:1); and amino acids 731 to 78 of BoNT/E (SEQ ID NO:1).

FIG. 7 shows proliferative responses of LNC (8×10⁵ cells/well) from BALB/c mice primed with 1 :g of BoNT/A toxoid to BoNT/A, BoNT/B and TeNT.

FIG. 8 shows proliferative responses of LNC (5×10⁵ cells/well) of Balb/c mice after 1 injection or after 3 injections with BoNT/A toxoid (1 :g/mouse/injection).

FIG. 9 shows proliferative responses of BoNT/A, BoNT/B and TeNT of LNC (7×10⁵ cells/well) from SJL mice primed with 1 :g BoNT/A toxoid.

FIG. 10 shows proliferative responses of LNC (5×10⁵ cells/well) of SJL mice to various synthetic BoNT/A peptides after 1 injection or after 3 injections with BoNT/A toxoid (1 :g/mouse/injection).

FIG. 11 shows binding of Balb/c anti-BoNT/A antibodies to BoNT/A and to overlapping synthetic peptides spanning the H_{N}-domain. Antisera were assayed at two dilutions (1:500 and 1:250, vol/vol).

FIG. 12 shows binding of SJL anti-BoNT/A antibodies to BoNT/A and to overlapping synthetic peptides of the H_{N}-chain. Antisera were assayed at dilutions of 1:500 and 1:250.

FIG. 13 shows a comparison of the binding profiles of BALB/c and SJL anti-BoNT/A toxoid antibodies at an antisera dilution of 1:250 (vol/vol), to BoNT/A and to overlapping synthetic peptides of the H_{N}-domain.

FIG. 14 shows protective activity of different dilutions of BALB/c and SJL anti-BoNT/A antisera. The results are expressed in percent survival to BoNT/A challenge versus antiserum dilution.

FIG. 15 shows protective activity of BALB/c and SJL anti-BoNT/A antisera obtained on day 36 after a first immunization. Antisera of each strain were tested at the indicated dilutions for their ability to protect recipient ICR mice against 1.05×LD₁₀₀ of active BoNT/A. The results are expressed in percent survival to BoNT/A challenge versus antiserum dilution.

FIG. 16 shows binding of BALB/c total antibodies in non-protecting (day 26) and protecting (day 36) anti-BoNT/A antisera to the overlapping synthetic peptides spanning the entire H chain and to the L-peptide around the enzyme active site of the L chain of BoNT/A. Results are from triplicate analyses and are expressed in net cpm, after correction for nonspecific binding in control wells coated with unrelated protein (BSA) or peptides and also controls of bound label to BoNT/A and to peptides in pre-immune serum of the same mice.

FIG. 17 shows binding of SJL total antibodies in non-protecting (day 26) and protecting (day 36) anti-BoNT/A antisera to the overlapping synthetic peptides spanning the entire H chain and to the L-peptide around the enzyme active site of the L chain of BoNT/A. Results are from triplicate analyses and are expressed in net cpm, after correction as described above.

FIG. 18 shows binding of BALB/c IgG antibodies in non-protecting (day 26) and protecting (day 36) anti-BoNT/A antisera to the overlapping synthetic peptides spanning the entire H chain and to the L-peptide around the enzyme active site of the L chain of BoNT/A. Results are from triplicate analyses and are expressed in net cpm, after correction as described above.

FIG. 19 shows binding of SJL IgG antibodies in non-protecting (day 26) and protecting (day 36) anti-BoNT/A antisera to the overlapping synthetic peptides spanning the entire H chain and to the L-peptide around the enzyme active site of the L chain of BoNT/A. Results are from triplicate analyses and are expressed in net cpm, after correction as described above.

FIG. 20 shows a comparison of IgG antibody binding profiles from protective (day 36) BALB/c and SJL antisera. The data are the same as those shown in FIGS. 4 and 5. Binding studies were performed with antisera at a dilution of 1:250 (vol/vol).

FIG. 21 shows binding to BoNT/A of antibodies in sera from CD patients (n=28) that are MPA-positive for anti-BoNT/A antibodies and in normal controls (n=10). Results are average of three experiments expressed in ratios of antibodies bound to BoNT/A over antibodies bound to negative controls.

FIG. 22 shows binding to BoNT/B of antibodies in MPA anti-BoNT/A positive sera from CD patients (n=28) and in normal controls (n=10). Results are in ratios of antibodies bound to BoNT/B over antibodies bound to negative controls.

FIG. 23 shows mapping of the antibody recognition profile in serum samples from 13 CD patients. Results are expressed as a ratio of antibodies bound to peptides in the CD sera/average of antibodies bound by four negative control peptides.

FIG. 24 shows mapping of the antibody recognition profile in serum samples from 15 CD patients. Results are expressed as a ratio of antibodies bound to peptides in the CD sera/average of antibodies bound by four negative control peptides.

FIG. 25 shows mapping of the antibody recognition profile in serum samples from 28 CD patients. Results are expressed as a ratio of antibodies bound to peptides in the CD sera/average of antibodies bound by four negative control peptides.

FIG. 26 shows binding to peptide N25 of antibodies in MPA-positive sera from CD patients (n=28) and in normal controls (n=10). Results are the average of four experiments and are expressed as a ratio of (antibodies bound to peptide N25)/(average of antibodies bound by negative control peptides N2, N12, C17 and C23).

FIG. 27 shows binding to peptide C10 of antibodies in MPA-positive sera from CD patients (n=28) and in normal controls (n=10). Results are the average of four experiments and are expressed as a ratio of (antibodies bound to peptide C10)/(average of antibodies bound by negative control peptides N2, N12, C17 and C23).

FIG. 28 shows binding to peptide C15 of antibodies in MPA-positive sera from CD patients (n=28) and in normal controls (n=10). Results are the average of four experiments and are expressed as a ratio of (antibodies bound to peptide C10)/(average of antibodies bound by negative control peptides N2, N12, C17 and C23).

FIG. 29 shows binding to peptide C31 of antibodies in MPA-positive sera from CD patients (n=28) and in normal controls (n=10). Results are the average of four experiments and are expressed as a ratio of (antibodies bound to peptide C10)/(average of antibodies bound by negative control peptides N2, N12, C17 and C23).

FIG. 30 shows binding to peptides (N25+C10) of antibodies in MPA-positive sera from CD patients (n=28) and in normal controls (n=10). The results, which are the average of four experiments, are expressed as a ratio of (antibodies bound to peptide N25+C10)/(average of antibodies bound by negative control peptides N2, N12, C17 and C23).

FIG. 31 shows binding peptides (N25+C10+C31) of antibodies in MPA-positive sera from CD patients (n=28) and in normal controls (n=10). Results, which are the average of four experiments, are expressed as a ratio of (antibodies bound to peptides N25+C10+C31)/(average of antibodies bound by negative control peptides N2, N12, C17 and C23).

FIG. 32 shows binding to peptides (N25+C10+C15) of antibodies in MPA-positive sera from CD patients (n=28) and in normal controls (n=10). Results, which are the average of four experiments, are expressed as a ratio of (antibodies bound to peptides N25+C10+C15)/(average of antibodies bound by negative control peptides N2, N12, C17 and C23).

FIG. 33 shows binding to peptides (N25+C10+C15+C31) of antibodies in MPA-positive sera from CD patients (n=28) and in normal controls (n=10). Results, which are the average of four experiments, are expressed as a ratio of (antibodies bound to peptides N25+C10+C15+C31)/(average of antibodies bound by negative control peptides N2, N12, C17 and C23).

FIG. 34 shows saturation curve experiments of ¹²⁵I-labeled BoNT/A to mouse synaptosomes. The experiments were carried out using 50,000 counts/minute (about 1 ng) of ¹²⁵I-labeled active BoNT/A peptide that was allowed to bind to different volumes of mouse a synaptosome preparation (from 0 to 8 μL).

FIG. 35 shows an inhibition of the binding of ¹²⁵I-labeled BoNT/A to mouse synaptosomes by unlabeled BoNT/A (●), or inactivate BoNT/A (▪) peptides. The experiments were carried out using 50,000 counts/minute (about 1 ng) of ¹²⁵I-labeled active BoNT/A peptide that was allowed to bind to 4 μL of synaptosomes in the presence of different amounts of either unlabeled active BoNT/A (●), or inactivate BoNT/A (▪). The levels of binding of ¹²⁵I-labeled toxin in the presence of different amounts of unlabeled toxin relative to the uninhibited controls were used to determine the percent of inhibition. The data are presented in percent binding in the presence of different concentrations of unlabeled BoNT/A (FIG. 35a) and as the percent inhibition values are plotted as a function of the reciprocal of inhibitor concentration (FIG. 35b).

FIG. 36 shows an inhibition of the binding of ¹²⁵I-labeled BoNT/A to mouse synaptosomes by unlabeled H_{N} and H_{C} BoNT/A peptides. The experiments were carried out using 50,000 counts/minute (about 1 ng) of ¹²⁵I-labeled active BoNT/A peptide that was allowed to bind to 4 μL of synaptosomes in the presence of different amounts of individual unlabeled H_{N} and H_{C} BoNT/A peptides. The levels of binding of ¹²⁵I-labeled toxin in the presence of different amounts of individual unlabeled H_{N} and H_{C} BoNT/A peptides relative to the uninhibited controls were used to determine the percent of inhibition. The data are presented in percent binding in the presence of different concentrations of individual unlabeled H_{N} and H_{C} BoNT/A peptides (FIG. 36a) and as the percent inhibition values are plotted as a function of the reciprocal of inhibitor concentration (FIG. 36b).

FIG. 37 shows the inhibition profile of the binding of ¹²⁵I-labeled BoNT/A to mouse synaptosomes by unlabeled H_{N} and H_{C} BoNT/A peptides. The experiments were carried out using 50,000 counts/minute (about 1 ng) of ¹²⁵I-labeled active BoNT/A peptide that was allowed to bind to 4 μL of synaptosomes in the presence of 1.0 μg of an individual unlabeled H-chain BoNT/A peptide. The levels of binding of ¹²⁵I-labeled toxin in the presence of an individual unlabeled H-chain BoNT/A peptide relative to the uninhibited controls were used to determine the percent of inhibition. The figure shows the values of maximum inhibition of each of the 60 H-chain peptides obtained by titrations. N1, amino acids 449 to 467 of SEQ ID NO: 1; N2, amino acids 463 to 481 of SEQ ID NO: 1; N3, amino acids 477 to 495 of SEQ ID NO: 1; N4, amino acids 491-509 of SEQ ID NO: 1; N5, amino acids 505 to 523 of SEQ ID NO: 1; N6, amino acids 519 to 537 of SEQ ID NO: 1; N7, amino acids 533 to 551 of SEQ ID NO: 1; N8, amino acids 547 to 565 of SEQ ID NO: 1; N9, amino acids 561 to 579 of SEQ ID NO: 1; N10, amino acids 575 to 593 of SEQ ID NO: 1; N11, amino acids 589 to 607 of SEQ ID NO: 1; N12, amino acids 603 to 621 of SEQ ID NO: 1; N13, amino acids 617 to 635 of SEQ ID NO: 1; N14, amino acids 631 to 649 of SEQ ID NO: 1; N15, amino acids 645 to 663 of SEQ ID NO: 1; N16, amino acids 659 to 677 of SEQ ID NO: 1; N17, amino acids 673 to 691 of SEQ ID NO: 1; N18, amino acids 687 to 705 of SEQ ID NO: 1; N19, amino acids 701 to 719 of SEQ ID NO: 1; N20, amino acids 715 to 733 of SEQ ID NO: 1; N21, amino acids 729 to 747 of SEQ ID NO: 1; N22, amino acids 743 to 761 of SEQ ID NO: 1; N23, amino acids 757 to 775 of SEQ ID NO: 1; N24, amino acids 771 to 789 of SEQ ID NO: 1; N25, amino acids 785 to 803 of SEQ ID NO: 1; N26, amino acids 799 to 817 of SEQ ID NO: 1; N27, amino acids 813 to 831 of SEQ ID NO: 1; N28, amino acids 827 to 845 of SEQ ID NO: 1; N29, amino acids 841 to 859 of SEQ ID NO: 1; C1, amino acids 855 to 873 of SEQ ID NO: 1; C2, amino acids 869 to 887 of SEQ ID NO: 1; C3, amino acids 883 to 901 of SEQ ID NO: 1; C4, amino acids 897 to 915 of SEQ ID NO: 1; C5, amino acids 911 to 929 of SEQ ID NO: 1; C6, amino acids 925 to 943 of SEQ ID NO: 1; C7, amino acids 939 to 957 of SEQ ID NO: 1; C8, amino acids 953 to 971 of SEQ ID NO: 1; C9, amino acids 967 to 985 of SEQ ID NO: 1; C10, amino acids 981 to 999 of SEQ ID NO: 1; C11, amino acids 995 to 1013 of SEQ ID NO: 1; C12, amino acids 1009 to 1027 of SEQ ID NO: 1; C13, amino acids 1023 to 1041 of SEQ ID NO: 1; C14, amino acids 1037 to 1055 of SEQ ID NO: 1; C15, amino acids 1051 to 1069 of SEQ ID NO: 1; C16, amino acids 1065 to 1083 of SEQ ID NO: 1; C17, amino acids 1079 to 1097 of SEQ ID NO: 1; C18, amino acids 1093 to 1111 of SEQ ID NO: 1; C19, amino acids 1107 to 1125 of SEQ ID NO: 1; C20, amino acids 1121 to 1139 of SEQ ID NO: 1; C21, amino acids 1135 to 1153 of SEQ ID NO: 1; C22, amino acids 1149 to 1167 of SEQ ID NO: 1; C23, amino acids 1163 to 1181 of SEQ ID NO: 1; C24, amino acids 1177 to 1195 of SEQ ID NO: 1; C25, amino acids 1191 to 1209 of SEQ ID NO: 1; C26, amino acids 1205 to 1223 of SEQ ID NO: 1; C27, amino acids 1219 to 1237 of SEQ ID NO: 1; C28, amino acids 1233 to 1251 of SEQ ID NO: 1; C29, amino acids 1247 to 1265 of SEQ ID NO: 1; C30, amino acids 1261 to 1279 of SEQ ID NO: 1; C31, amino acids 1275 to 1296 of SEQ ID NO: 1.

### DETAILED DESCRIPTION OF THE INVENTION

Introduction

This invention relates to botulinum neurotoxin A (BoNT/A) peptides that represent the complete repertoire of epitopes from the H_{N} domain and H_{C} domain of BoNT/A recognized by antibodies from humans immunized with pentavalent botulinum toxoid. BoNT/A peptides of the invention, and antibodies that bind to such peptides, are useful, for example, in methods for predicting or diagnosing immunoresistance to botulinum toxin therapy, for reducing the development of such immunoresistance, and for boosting immunity against unwanted botulinum toxicity.

Botulinum neurotoxins (BoNTs) are a group of protein neurotoxins produced by Clostridium botulinum that are among the most toxic substances known to man. Seven immunologically distinct BoNT serotypes (A through G) are known, including two subtypes of type C (C1 and C2). Botulinum neurotoxins are synthesized from a single polypeptide chain with a molecular weight of about 150 KDa, which is activated after secretion by nicking of a single peptide bond by an endogenous or exogenous protease. In C. botulinum strains that produce BoNTs A, C, D, and some types of B and F, the proteolytic enzyme is endogenous, while in other strains such as those that produce type E and some types B and F, the proteolytic enzyme is exogenous. The nicking of the progenitor toxin generally results in generation of a di-chain molecule of two subunits, a 100 KDa heavy chain (HC) and a 50 KDa light chain (LC). With the exception of BoNT/C2, the two subunits are held together by a disulfide bond, which is important for neurotoxicity of toxin added extracellularly.

Each mature di-chain molecule comprises three functionally distinct domains: 1) an enzymatic domain located in the LC that includes a metalloprotease region containing a zinc-dependent endopeptidase activity which specifically targets core components of the neurotransmitter release apparatus; 2) a translocational domain contained within the amino-terminal half of the H_{C} (denoted H_{N} domain) that facilitates release of the toxin from intracellular vesicles into the cytoplasm of the target cell; and 3) a binding domain found within the carboxy-terminal half of the H_{C} (denoted H_{C} domain) that determines the binding activity and binding specificity of the toxin to the acceptor complex located at the surface of the target cell.

The overall cellular intoxication mechanism whereby the seven BoNT serotypes enter a neuron and inhibit neurotransmitter release is similar and can be described in four steps: 1) membrane binding, 2) complex internalization, 3) light chain translocation, and 4) exocytosis inhibition. The process is initiated when the H_{C} domain of a BoNT binds to BoNT-specific acceptor complex located on the plasma membrane surface of a target cell. The binding specificity of an acceptor complex is thought to be achieved by specific combinations of gangliosides and protein receptors that appear to distinctly comprise each BoNT serotype-specific acceptor. Once bound, the BoNT/acceptor complexes are internalized by endocytosis and the internalized vesicles are sorted to specific intracellular routes. The translocation step appears to be triggered by the acidification of the vesicle compartment. This process seems to initiate two important pH-dependent structural rearrangements that increase hydrophobicity and promote enzymatic activation of the toxin. Once activated, light chain endopeptidase of the toxin is released from the intracellular vesicle into the cytosol where it specifically targets one of three known core components of the neurotransmitter release apparatus. These core proteins [vesicle-associated membrane protein (VAMP)/synaptobrevin, synaptosomal-associated protein of 25 kDa (SNAP-25) and syntaxin] are necessary for synaptic vesicle docking and fusion at the nerve terminal and constitute the synaptic members of the soluble N-ethylmaleimide-sensitive factor-attachment protein-receptor (SNARE) family. The selective proteolysis of synaptic SNAREs accounts for the total block of neurotransmitter release caused by BoNTs in vivo. For greater details see, e.g., Humeau, supra, 2000; Turton, supra, 2002; Atassi, supra, 2003; Lalli, supra, 2003, which are hereby incorporated by reference.

The complete primary structures of BoNTs A through G have been determined, see, e.g., Thomas Binz et al., The Complete Sequence of Botulinum Neurotoxin Type A and Comparison with Other Clostridial Neurotoxins, 265(16) J. BIOL. CHEM. 9153-9158 (1990); A. Willems et al., Sequence of the Gene Coding for the Neurotoxin of Clostridium Botulinum Type A Associated With Infant Botulism: Comparison With Other Clostridial Neurotoxins, 144(7) RES. MICROBIOL. 547-556 (1993); R. A. Hutson et al., Nucleotide Sequence of the Gene Coding for Non-Proteolytic Clostridium Botulinum Type B Neurotoxin: Comparison With Other Clostridial Neurotoxins, 28(2) CURR. MICROBIOL. 101-110 (1994); Kathryn D. Campbell et al., Gene Probes For Identification of the Botulinal Neurotoxin Gene and Specific Identification of Neurotoxin Types B, E, And F, 31(9) J. CLIN. MICROBIOL. 2255-2262 (1993); Daniel Hauser et al., Nucleotide Sequence of Clostridium botulinum C1 Neurotoxin, 18(16) NUCLEIC ACIDS RES. 4924 (1990); Daniel Hauser et al., Comparative Analysis of C3 and Botulinal Neurotoxin Genes and Their Environment in Clostridium Botulinum Types C and D, 175(22) J. BACTERIOL. 7260-7268 (1993); K. Kimura et al., The Complete Nucleotide Sequence of the Gene Coding for Botulinum Type C1 Toxin in the C-ST Phage Genome, 171(3) BIOCHEM. BIOPHYS. RES. COMMUN. 1304-1311 (1990); K. Kimura et al., Cloning of the Structural Gene for Clostridium Botulinum Type C1 Toxin and Whole Nucleotide Sequence of its Light Chain Component, 57(4) APPL. ENVIRON. MICROBIOL. 1168-1172 (1991); Daniel Hauser et al., Botulinal Neurotoxin C1 Complex Genes, Clostridial Neurotoxin Homology and Genetic Transfer in Clostridium botulinum, 33(4) TOXICON 515-526 (1995); Thomas Binz et al., Nucleotide Sequence of the Gene Encoding Clostridium Botulinum Neurotoxin Type D, 18(18) Nucleic Acids Res. 5556 (1990); H. Sunagawa et al., The Complete Amino Acid Sequence of the Clostridium Botulinum Type D Neurotoxin, Deduced by Nucleotide Sequence Analysis of the Encoding Phage D-16 Phi Genome, 54(5) J. VET. MED. SCI. 905-913 (1992); S. Poulet et al., Sequences of the Botulinal Neurotoxin E Derived From Clostridium Botulinum Type E (Strain Beluga) and Clostridium Butyricum (Strains ATCC 43181 And ATCC43755), 183(1) BIOCHEM. BIOPHYS. RES. COMMUN. 107-113 (1992); Sarah M. Whelan et al., The Complete Amino Acid Sequence of the Clostridium Botulinum Type-E Neurotoxin, Derived by Nucleotide-Sequence Analysis of the Encoding Gene, 204(2) EUR. J. BIOCHEM. 657-667 (1992); Alison K. East et al., Sequence of the Gene Encoding Type F Neurotoxin of Clostridium botulinum, 75(2-3) FEMS MICROBIOL. LETT. 225-230 (1992); and Kathryn D. Campbell et al., Nucleotide Sequence of the Gene Coding for Clostridium Botulinum (Clostridium argentinense) Type G Neurotoxin: Genealogical Comparison With Other Clostridial Neurotoxins, 1216(3) BIOCHIM. BIOPHYS. ACTA. 487-491 (1993). In addition, the disulfide pairing in BoNT/A has been determined. Several regions of homology exist within the amino acid sequences of the different serotypes of BoNT, as described in, e.g., M. Zouhair Atassi & Minako Oshima, Structure, Activity, and Immune (T and B Cell) Recognition of Botulinum Neurotoxins, 19(3) CRIT. REV. IMMUNOL. 219-260 (1999).

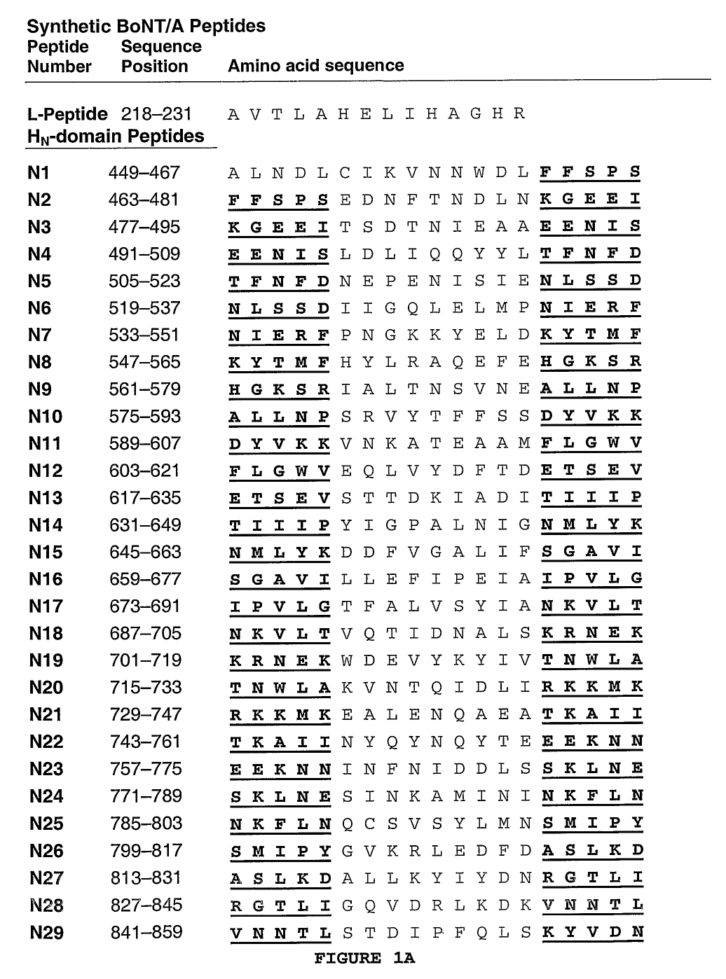

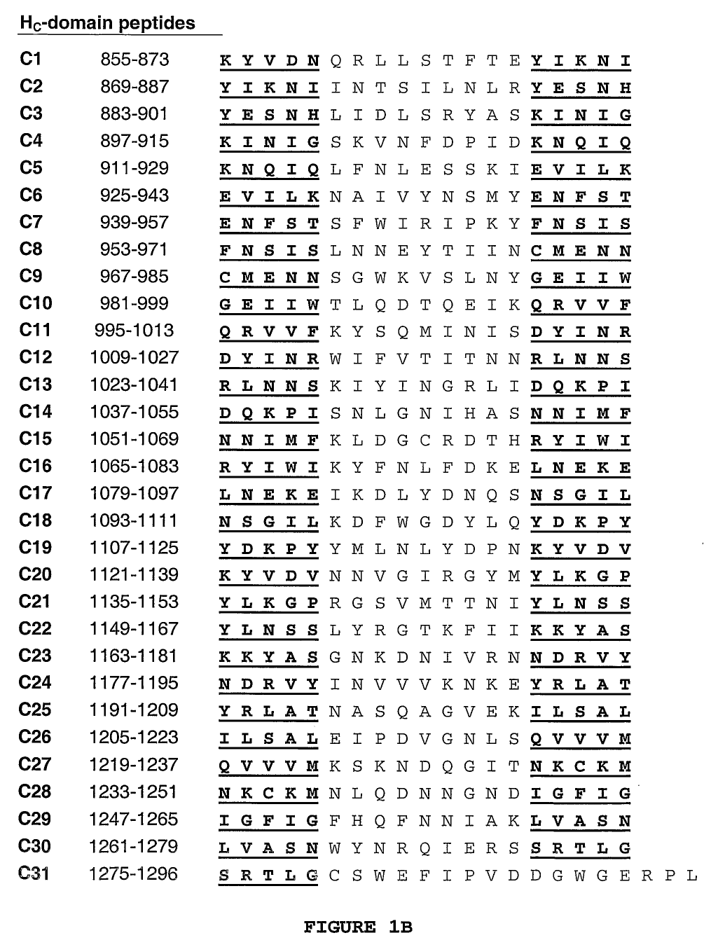

The present invention relates to the discovery of small BoNT/A peptides which elicit antibody responses and represent the repertoire of epitopes found within both the BoNT/A H_{N} domain and H_{C} domain recognized by four animal species, including humans. As shown herein in Examples 1, 2, 3, 4 & 5, antigenic regions of both domains were mapped using human, horse, mouse and chicken sera obtained following immunization with BoNT/A. Mapping was performed using twenty-nine synthetic BoNT/A peptides, each containing nineteen residues, that overlap consecutively by five residues and correspond to the entire length of the H_{N} domain and thirty-one synthetic BoNT/A peptides, each containing nineteen residues, that overlap consecutively by five residues and correspond to the entire length of the H_{C} domain, with the exception of C31, which is twenty-two residues in length. The amino acid sequences of the sixty peptides used for mapping are shown in FIG. 1A. Results from the mapping studies revealed 1) nineteen segments of BoNT/A that represent the complete repertoire of continuous antigenic regions on the BoNT/A H_{N} domain; and 2) 1) nineteen segments of BoNT/A that represent the complete repertoire of continuous antigenic regions on the BoNT/A H_{C} domain, see, e.g., Examples 1, 2, 3, 4, & 5.

As disclosed herein in Example 7, T- and B-cell recognition profiles of the BoNT/A H_{N} domain were mapped in two inbred mouse strains, BALB/c (H-2^{d}) and SJL (H-2^{s}), that are high responders to BoNT/A. As summarized in Table 5, the results obtained with the two high-responder mouse strains demonstrate that responses to each antibody and T cell epitope are under separate genetic control and further indicate that there is partial overlap between antibody and T cell H_{N} recognition regions.

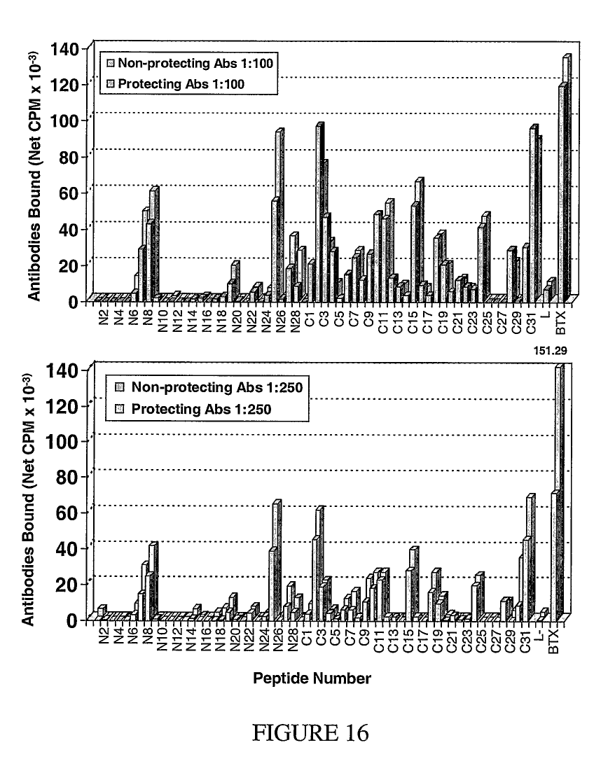

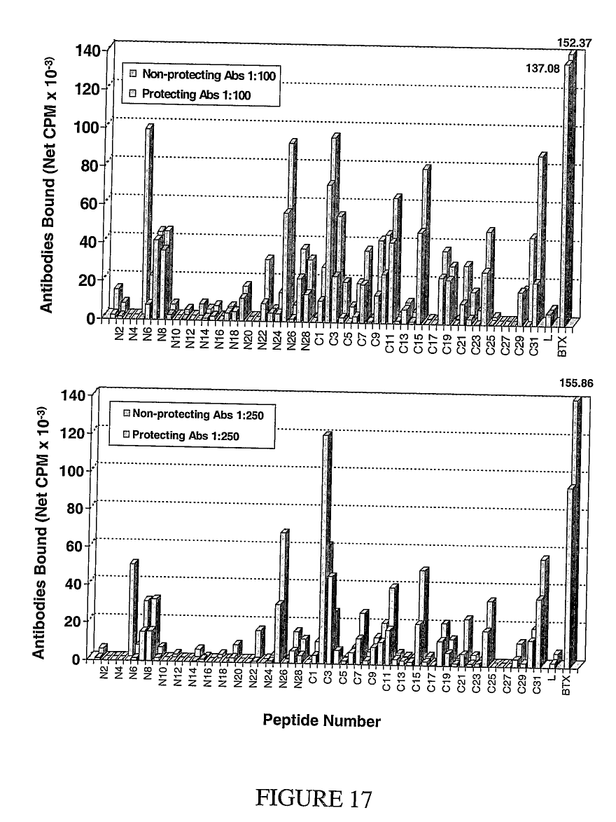

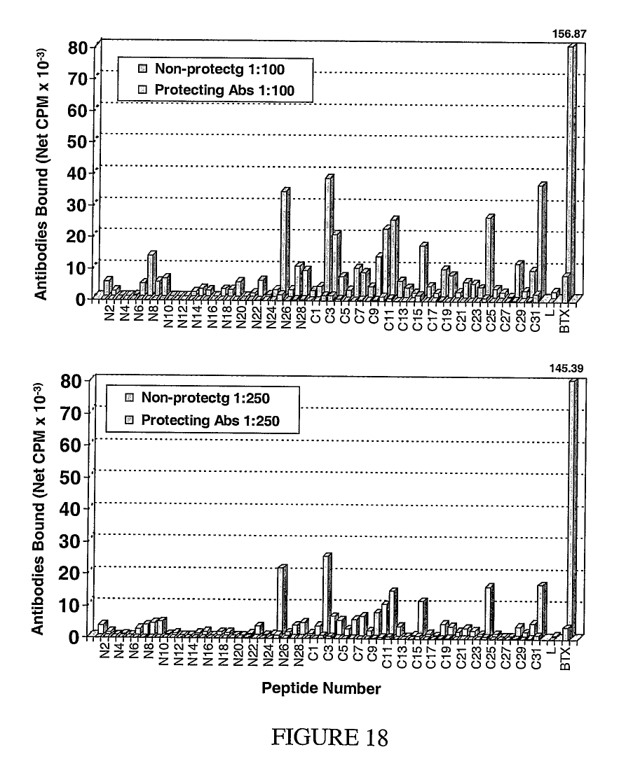

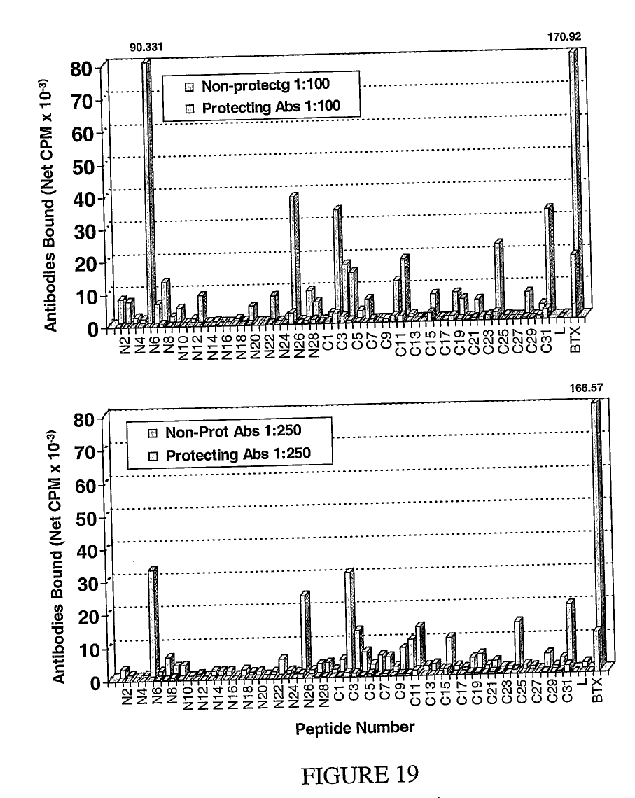

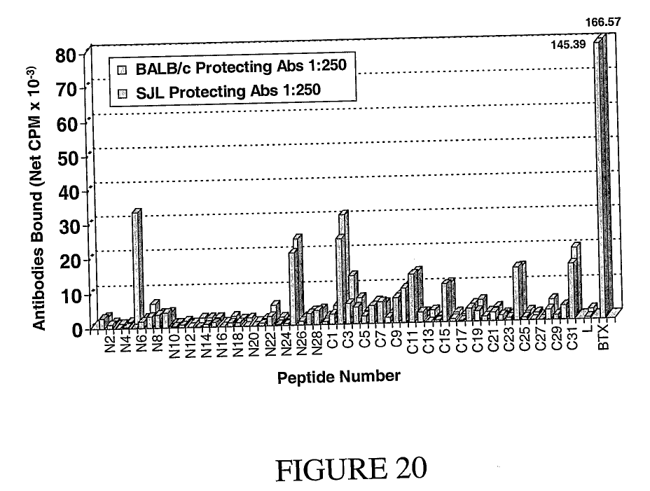

Resistance in the majority of patients is associated with the appearance of blocking anti-toxin antibodies in patient serum (Hilke Göschel et al., Botulinum A Toxin Therapy: Neutralizing and Nonneutralizing Abs—Therapeutic Consequences, 147(1) EXP. NEUROL. 96-102, (1997); Atassi & Oshima, supra, 1999; Jankovic, supra, 2002. While all patient antibody responses against the toxin are not observed initially, additional injections of toxin appear to cause a switch of the non-blocking antibodies in the patient's serum to blocking antibodies. As further disclosed herein in Example 8, the epitope recognition profile was compared in inbred BALB/c and SJL mice before and after the switch from production of non-protective to protective antibodies. The results disclosed herein demonstrated only slight differences in the epitope recognition profiles of non-protective and protective antisera, indicating that changes in antibody binding may not always protection, or lack thereof, by serum from a given strain (FIGS. 16 and 17). Furthermore, as shown in FIGS. 18 and 19, IgG antibodies in the protective antisera of each mouse strain bound to the same peptides as did total antibodies (IgG and IgM) in the same serum, while in both mouse strains, non-protective antisera contained few, if any, IgG antibodies to these peptides. These results appear to indicate that protection can be a function of immunoglobulin class, with IgG antibodies conferring protection against botulinum toxin.

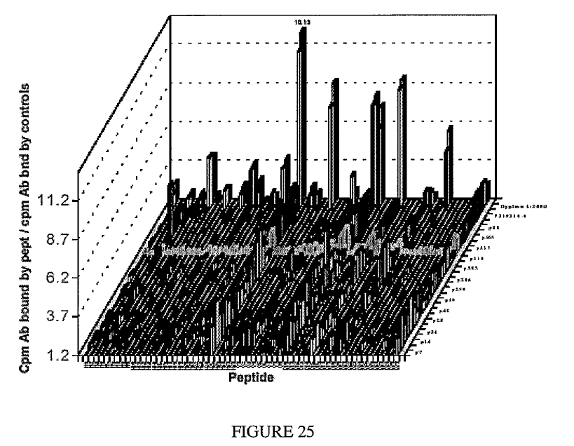

Additional studies disclosed herein in Example 9 demonstrate that in vitro binding assays performed in the presence of excess tetanus toxoid can be used to determine the levels of blocking or protective anti-BoNT/A antibodies in human serum samples. In particular, sera from 28 cervical dystonia patients containing protective antibodies as indicated by the mouse protection assay (MPA) and 10 negative sera controls from unimmunized human were analyzed. As shown in FIGS. 24 to 26 and summarized in Table 6, peptides which bound antibodies in MPA-positive human patient sera also bound antibodies in hyperimmune mouse sera, while the antibody-binding profile of patient sera was more restricted than the profile of the hyperimmune sera. As further disclosed herein in Example 9, several peptides bound antibodies in most patient samples, with 25 out of 28 sera containing antibodies that bound peptide N25; 24 out of 28 sera containing antibodies that bound peptide C10; and lower binding to peptides C15, C20 and C31 seen in the majority of patient samples. These results indicate that, while there is some variability among the peptide-binding profiles of MPA-positive human sera, several synthetic BoNT/A peptides bind antibodies in the large majority of human patient sera that contain protective antibodies.

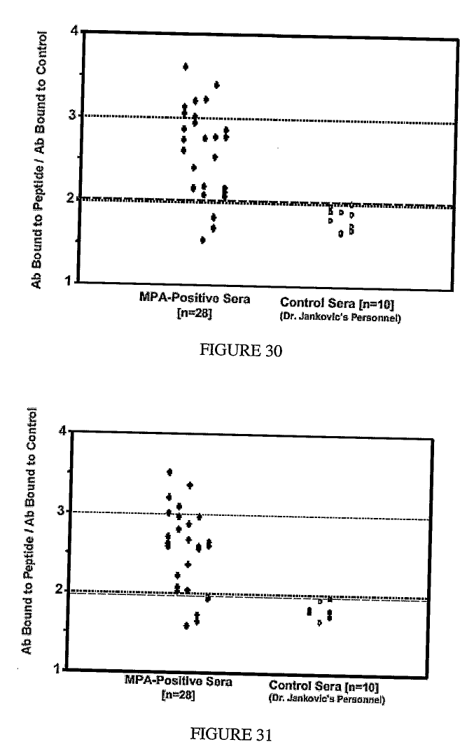

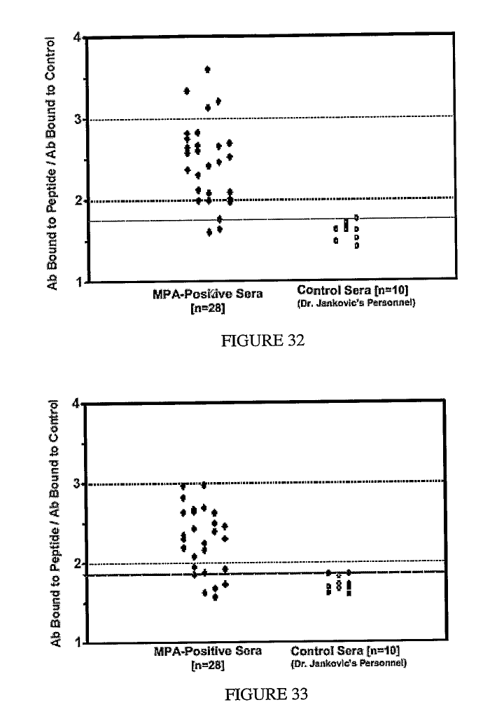

Further results disclosed herein demonstrate that an assay based on a combination of two or more synthetic BoNT/A peptides can be useful for detecting the presence of protective or blocking antibodies in the sera of patients treated with a BoNT/A formulation. As shown in FIG. 30, in an assay combining synthetic peptides N25 and C10, 25 out of 28 (89.3%) of the MPA-positive CD sera were discriminated from control sera. FIG. 32 shows that a combination of the synthetic peptides N25, C10 and C15 also served to distinguish 25 out of 28 (89.3%) of the MPA-positive CD sera from controls. Thus, the results disclosed herein demonstrate that a combination assay using peptides N25 and C10, or peptides N25, C10 and C15 can be useful for detecting the presence of specific anti-toxin antibodies in BOTOX® treated patients. Furthermore, one or a combination of the synthetic peptides N25, C10, N15, N20 or N31, or a conservative variant or immunoreactive fragment thereof, also can be useful in a variety of diagnostic or therapeutic applications including, without limitation, methods of predicting or determining immunoresistance to botulinum toxin therapy; methods of preventing or reducing immunoresistance to botulinum toxin therapy and related tolerogenic compositions; methods of vaccinating against botulinum toxin and related vaccine compositions; methods of removing anti-botulinum toxin antibodies from blood, plasma or serum and affinity-matrices useful therefore; and new therapeutic formulations for blocking the effect of neutralizing antibodies in situ. Such therapeutic formulations include excess synthetic protective antibody-binding peptides together with the active toxin formulation.

As mentioned above, the first step in the intoxication process is the binding of BoNT/A to a cell surface acceptor complex containing BoNT/A-specific receptor proteins and gangliosides. Using the sixty BoNT/A peptides regions necessary for the binding of the toin to the acceptor complex were identified, see, e.g., Example 10. Results from these mapping studies revealed 1) eleven segments of BoNT/A that represent the complete repertoire of continuous antigenic regions on the BoNT/A H_{N} domain; and 2) 1) eight segments of BoNT/A that represent the complete repertoire of continuous antigenic regions on the BoNT/A H_{C} domain, see, e.g., Examples 10.

II. BoNT/A Peptide Compositions

The present invention provides a BoNT/A peptide having a length of at most 60 amino acids and consisting of at least 5 contiguous amino acids selected from one of the following BoNT/A amino acid sequences: 449-467 of SEQ ID NO:1(N1), 463-481 of SEQ ID NO:1 (N2), 491-509 of SEQ ID NO:1 (N4), 505-523 of SEQ ID NO:1 (N5), 519-537 of SEQ ID NO:1 (N6), 533-551 of SEQ ID NO:1 (N7), 547-565 of SEQ ID NO:1 (N8), 561-579 of SEQ ID NO:1 (N9), 575-593 of SEQ ID NO:l (N10), 589-607 of SEQ ID NO:1 (N11), 631-649 of SEQ ID NO:1 (N14), 645-663 of SEQ ID NO:1 (N15), 659-677 of SEQ ID NO:1 (N16), 673-691 of SEQ ID. NO:1 (N17), 701-719 of SEQ ID NO:1 (N19), 715-733 of SEQ ID NO:1 (N20), 729-747 of SEQ ID NO:1 (N21), 743-761 of SEQ ID NO:1 (N22), 757-775 of SEQ ID NO:1 (N23), 771-789 of SEQ ID NO:1 (N24), 785-803 of SEQ ID NO:1 (N25), 799-817 of SEQ ID NO:1 (N26), 813-831 of SEQ ID NO:1 (N27), 827-845 of SEQ ID NO:1 (N28), 869-887 of SEQ ID NO:1 (C2), 883-901 of SEQ ID NO:1 (C3), 911-929 of SEQ ID NO:1 (C5), 925-943 of SEQ ID NO:1 (C6), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1065-1083 of SEQ ID NO:1 (C16), 1079-1097 of SEQ ID NO:1 (C17), 1107-1125 of SEQ ID NO:1 (C19), 1121-1139 of SEQ ID NO:1 (C20), 1135-1153 of SEQ ID NO:1 (C21), 1149-1167 of SEQ ID NO:1 (C22), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28), 1247-1265 of SEQ ID NO:1 (C29), 1261-1279 of SEQ ID NO:1 (C30), or 1275-1296 of SEQ ID NO:1 (C31), or a conservative variant, or a non-conservative variant, or immunoreactive fragment thereof, with the proviso that the BoNT/A peptide is not SEQ ID NO:2.

In one embodiment of the present invention, a BoNT/A peptide has a length of at most 60 amino acids and consists of at least 5 contiguous amino acids selected from one of the following BoNT/A amino acid sequences: 449-467 of SEQ ID NO:1(N1), 463-481 of SEQ ID NO:1 (N2), 491-509 of SEQ ID NO:1 (N4), 505-523 of SEQ ID NO:1 (N5), 519-537 of SEQ ID NO:1 (N6), 533-551 of SEQ ID NO:1 (N7), 547-565 of SEQ ID NO:1 (N8), 561-579 of SEQ ID NO:1 (N9), 575-593 of SEQ ID NO:1 (N10), 589-607 of SEQ ID NO:1 (N11), 631-649 of SEQ ID NO:1 (N14), 645-663 of SEQ ID NO:1 (N15), 659-677 of SEQ ID NO:1 (N16), 673-691 of SEQ ID NO:1 (N17), 701-719 of SEQ ID NO:1 (N19), 715-733 of SEQ ID NO:1 (N20), 729-747 of SEQ ID NO:1 (N21), 743-761 of SEQ ID NO:1 (N22), 757-775 of SEQ ID NO:1 (N23), 771-789 of SEQ ID NO:1 (N24), 785-803 of SEQ ID NO:1 (N25), 799-817 of SEQ ID NO:1 (N26), 813-831 of SEQ ID NO:1 (N27), 827-845 of SEQ ID NO:1 (N28), 869-887 of SEQ ID NO:1 (C2), 883-901 of SEQ ID NO:1 (C3), 911-929 of SEQ ID NO:1 (C5), 925-943 of SEQ ID NO:1 (C6), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:l (C15), 1065-1083 of SEQ ID NO:1 (C16), 1079-1097 of SEQ ID NO:1 (C17), 1107-1125 of SEQ ID NO:1 (C19), 1121-1139 of SEQ ID NO:1 (C20), 1135-1153 of SEQ ID NO:1 (C21), 1149-1167 of SEQ ID NO:1 (C22), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28), 1247-1265 of SEQ ID NO:1 (C29), 1261-1279 of SEQ ID NO:1 (C30), or 1275-1296 of SEQ ID NO:1 (C31). In an aspect of this embodiment, such a BoNT/A peptide is selected from one of the following amino acid sequences: 463-481 of SEQ ID NO:1 (N2), 519-537 of SEQ ID NO:1 (N6), 533-551 of SEQ ID NO:1 (N7), 547-565 of SEQ ID NO:1 (N8), 561-579 of SEQ ID NO:1 (N9), 589-607 of SEQ ID NO:1 (N11), 659-677 of SEQ ID NO:1 (N1 6), 743-761 of SEQ ID NO:1 (N22), 785-803 of SEQ ID NO:1 (N25), 813-831 of SEQ ID NO:1 (N27), 827-845 of SEQ ID NO:1 (N28), 869-887 of SEQ ID NO:1 (C2), 883-901 of SEQ ID NO:1 (C3), 911 -929 of SEQ ID NO:1 (C6), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1121-1139 of SEQ ID NO:1 (C20), 1135-1153 of SEQ ID NO:1 (C21), 1177-1195 of SEQ ID NO:1 (C24), 1261-1279 of SEQ ID NO:l (C30) or 1275-1296 of SEQ ID NO:1 (C31). In another aspect of this embodiment, such a BoNT/A peptide is selected from one of the following amino acid sequences: 463-481 of SEQ ID NO:1 (N2), 533-551 of SEQ ID NO:1 (N7), 547-565 of SEQ ID NO:1 (N8), 561-579 of SEQ ID NO:1 (N9), 659-677 of SEQ ID NO:1 (N16), 743-761 of SEQ ID NO:1 (N22), 785-803 of SEQ ID NO:1 (N25), 813-831 of SEQ ID NO:1 (N27), 883-901 of SEQ ID NO:1 (C3), 939-957 of SEQ ID NO:1 (C7), 967-985 of SEQ ID NO:1 (C9), 981-999 of SEQ ID NO:1 (C1 0), 995-1013 of SEQ ID NO:1 (C11), 1051-1069 of SEQ ID NO:1 (C15), 1121-1139 of SEQ ID NO:1 (C20) or 1177-1195 of SEQ ID NO:1 (C24) or 1275-1296 of SEQ ID NO: 1 (C31). In another aspect of this embodiment, such a BoNT/A peptide is selected from one of the following amino acid sequences: 533-551 of SEQ ID NO: 1 (N7), 547-565 of SEQ ID NO: 1 (N8), 743-761 of SEQ ID NO: 1 (N22), 785-803 of SEQ ID NO: 1 (N25), 813-831 of SEQ ID NO: 1 (N27), 995-1013 of SEQ ID NO: 1 (C11); 1051-1069 of SEQ ID NO: 1 (C15), 1177-1195 of SEQ ID NO: 1 (C24), or 1275-1296 of SEQ ID NO: 1 (C31).

In yet another aspect of this embodiment, a BoNT/A peptide has a length of at most 60 amino acids and consists of at least 5 contiguous amino acids selected from one of the following BoNT/A amino acid sequences: 463-481 of SEQ ID NO:1 (N2), 505-523 of SEQ ID NO:1 (N5), 519-537 of SEQ ID NO:1 (N6), 533-551 of SEQ ID NO:1 (N7), 603-621 of SEQ ID NO:1 (N12), 645-663 of SEQ ID NO:1 (N15), 659-677 of SEQ ID NO:1 (N16), 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1079-1097 of SEQ ID NO:1 (C17), 1107-1125 of SEQ ID NO:1 (C19), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28) or 1275-1296 of SEQ ID NO:1 (C31). In yet another aspect of this embodiment, such a BoNT/A peptide is selected from one of the following amino acid sequences: 533-551 of SEQ ID NO:1 (N7), 659-677 of SEQ ID NO:1 (N16), 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1107-1125 of SEQ ID NO:1 (C19), 1163-1181 of SEQ ID NO:1 (C23), 1191-1209 of SEQ ID NO:1 (C25), 1233-1251 of SEQ ID NO:1 (C28) or 1275-1296 of SEQ ID NO:1 (C31). In yet another aspect of this embodiment, such a BoNT/A peptide is selected from one of the following amino acid sequences: 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1079-1097 of SEQ ID NO:1 (C17), 1107-1125 of SEQ ID NO:1 (C19), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28) or 1275-1296 of SEQ ID NO:1 (C31). In yet another aspect of this embodiment, such a BoNT/A peptide is selected from one of the following amino acid sequences: 729-747 of SEQ ID NO:1 (N21) 799-817 of SEQ ID NO:1 (N26), 1065-1083 of SEQ ID NO:1 (C16), 1163-1181 of SEQ ID NO:1 (C23) or 1275-1296 of SEQ ID NO:1 (C31).

In another embodiment of the present invention, a BoNT/A composition comprises a BoNT/A peptide that has one of the following BoNT/A amino acid sequences: 449-467 of SEQ ID NO:1(N1), 463-481 of SEQ ID NO:1 (N2), 491-509 of SEQ ID NO:1 (N4), 505-523 of SEQ ID NO:1 (N5), 519-537 of SEQ ID NO:1 (N6), 533-551 of SEQ ID NO:1 (N7), 547-565 of SEQ ID NO:1 (N8), 561-579 of SEQ ID NO:1 (N9), 575-593 of SEQ ID NO:1 (N10), 589-607 of SEQ ID NO:1 (N11), 631-649 of SEQ ID NO:1 (N14), 645-663 of SEQ ID NO:1 (N15), 659-677 of SEQ ID NO:1 (N16), 673-691 of SEQ ID NO:1 (N17), 701-719 of SEQ ID NO:1 (N19), 715-733 of SEQ ID NO:1 (N20), 729-747 of SEQ ID NO:1 (N21), 743-761 of SEQ ID NO:1 (N22), 757-775 of SEQ ID NO:1 (N23), 771-789 of SEQ ID NO:1 (N24), 785-803 of SEQ ID NO:1 (N25), 799-817 of SEQ ID NO:1 (N26), 813-831 of SEQ ID NO:1 (N27), 827-845 of SEQ ID NO:1 (N28), 869-887 of SEQ ID NO:1 (C2), 883-901 of SEQ ID NO:1 (C3), 911-929 of SEQ ID NO:1 (C5), 925-943 of SEQ ID NO:1 (C6), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1065-1083 of SEQ ID NO:1 (C16), 1079-1097 of SEQ ID NO:1 (C17), 1107-1125 of SEQ ID NO:1 (C19), 1121-1139 of SEQ ID NO:1 (C20), 1135-1153 of SEQ ID NO:1 (C21), 1149-1167 of SEQ ID NO:1 (C22), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28), 1247-1265 of SEQ ID NO:1 (C29), 1261-1279 of SEQ ID NO:1 (C30), or 1275-1296 of SEQ ID NO:1 (C31). In an aspect of this embodiment, such a BoNT/A peptide is selected from one of the following amino acid sequences: 463-481 of SEQ ID NO:1 (N2), 519-537 of SEQ ID NO:1 (N6), 533-551 of SEQ ID NO:1 (N7), 547-565 of SEQ ID NO:1 (N8), 561-579 of SEQ ID NO:1 (N9), 589-607 of SEQ ID NO:1 (N11), 659-677 of SEQ ID NO:1 (N16), 743-761 of SEQ ID NO:1 (N22), 785-803 of SEQ ID NO:1 (N25), 813-831 of SEQ ID NO:1 (N27), 827-845 of SEQ ID NO:1 (N28), 869-887 of SEQ ID NO:1 (C2), 883-901 of SEQ ID NO:1 (C3), 911-929 of SEQ ID NO:1 (C6), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1121-1139 of SEQ ID NO:1 (C20), 1135-1153 of SEQ ID NO:1 (C21), 1177-1195 of SEQ ID NO:l (C24), 1261-1279 of SEQ ID NO:1 (C30) or 1275-1296 of SEQ ID NO:1 (C31). In another aspect of this embodiment, such a BoNT/A peptide is selected from one of the following amino acid sequences: 463-481 of SEQ ID NO:1 (N2), 533-551 of SEQ ID NO:1 (N7), 547-565 of SEQ ID NO:1 (N8), 561-579 of SEQ ID NO:1 (N9), 659-677 of SEQ ID NO:1 (N16), 743-761 of SEQ ID NO:1 (N22), 785-803 of SEQ ID NO:1 (N25), 813-831 of SEQ ID NO:1 (N27), 883-901 of SEQ ID NO:1 (C3), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1121-1139 of SEQ ID NO:1 (C20) or 1177-1195 of SEQ ID NO:1 (C24) or 1275-1296 of SEQ ID NO: 1 (C31). In another aspect of this embodiment, such a BoNT/A peptide is selected from one of the following amino acid sequences: 533-551 of SEQ ID NO: 1 (N7), 547-565 of SEQ ID NO: 1 (N8), 743-761 of SEQ ID NO: 1 (N22), 785-803 of SEQ ID NO: 1 (N25), 813-831 of SEQ ID NO: 1 (N27), 995-1013 of SEQ ID NO: 1 (C11); 1051-1069 of SEQ ID NO: 1 (Cl 5), 1177-1195 of SEQ ID NO: 1 (C24), or 1275-1296 of SEQ ID NO: 1 (C31).

In yet another aspect of this embodiment, a BoNT/A composition comprises a BoNT/A peptide that has one of the following BoNT/A amino acid sequences: 463-481 of SEQ ID NO:1 (N2), 505-523 of SEQ ID NO:1 (N5), 519-537 of SEQ ID NO:1 (N6), 533-551 of SEQ ID NO:1 (N7),603-621 of SEQ ID NO:1 (N12), 645-663 of SEQ ID NO:1 (N15), 659-677 of SEQ ID NO:1 (N16), 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1079-1097 of SEQ ID NO:1 (C17), 1107-1125 of SEQ ID NO:1 (C19), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28) or 1275-1296 of SEQ ID NO:1 (C31). In yet another aspect of this embodiment, such a BoNT/A peptide is selected from one of the following amino acid sequences: 533-551 of SEQ ID NO:1 (N7), 659-677 of SEQ ID NO:1 (N16), 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1107-1125 of SEQ ID NO:1 (C19), 1163-1181 of SEQ ID NO:1 (C23), 1191-1209 of SEQ ID NO:1 (C25), 1233-1251 of SEQ ID NO:1 (C28) or 1275-1296 of SEQ ID NO:1 (C31). In yet another aspect of this embodiment, such a BoNT/A peptide is selected from one of the following amino acid sequences: 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1079-1097 of SEQ ID NO:1 (C17), 1107-1125 of SEQ ID NO:1 (C19), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28) or 1275-1296 of SEQ ID NO:1 (C31). In yet another aspect of this embodiment, such a BoNT/A peptide is selected from one of the following amino acid sequences: 729-747 of SEQ ID NO:1 (N21) 799-817 of SEQ ID NO:1 (N26), 1065-1083 of SEQ ID NO:1 (C16), 1163-1181 of SEQ ID NO:1 (C23) or 1275-1296 of SEQ ID NO:1 (C31).

In yet another embodiment of the present invention, a BoNT/A peptide has a length of at most 60 amino acids and consists of at least 5 contiguous amino acids selected from one of the following BoNT/A amino acid sequences: 449-467 of SEQ ID NO:1(N1), 463-481 of SEQ ID NO:1 (N2), 491-509 of SEQ ID NO:1 (N4), 505-523 of SEQ ID NO:1 (N5), 519-537 of SEQ ID NO:1 (N6), 533-551 of SEQ ID NO:1 (N7), 547-565 of SEQ ID NO:1 (N8), 561-579 of SEQ ID NO:1 (N9), 575-593 of SEQ ID NO:1 (N10), 589-607 of SEQ ID NO:1 (N11), 631-649 of SEQ ID NO:1 (N14), 645-663 of SEQ ID NO:1 (N15), 659-677 of SEQ ID NO:1 (N16), 673-691 of SEQ ID NO:1 (N17), 701-719 of SEQ ID NO:1 (N19), 715-733 of SEQ ID NO:1 (N20), 729-747 of SEQ ID NO:1 (N21), 743-761 of SEQ ID NO:1 (N22), 757-775 of SEQ ID NO:1 (N23), 771-789 of SEQ ID NO:1 (N24), 785-803 of SEQ ID NO:1 (N25), 799-817 of SEQ ID NO:1 (N26), 813-831 of SEQ ID NO:1 (N27), 827-845 of SEQ ID NO:1 (N28), 869-887 of SEQ ID NO:1 (C2), 883-901 of SEQ ID NO:1 (C3), 911-929 of SEQ ID NO:1 (C5), 925-943 of SEQ ID NO:1 (C6), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1065-1083 of SEQ ID NO:1 (C16), 1079-1097 of SEQ ID NO:1 (C17), 1107-1125 of SEQ ID NO:1 (C19), 1121-1139 of SEQ ID NO:1 (C20), 1135-1153 of SEQ ID NO:1 (C21), 1149-1167 of SEQ ID NO:1 (C22), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28), 1247-1265 of SEQ ID NO:1 (C29), 1261-1279 of SEQ ID NO:1 (C30), or 1275-1296 of SEQ ID NO:1 (C31), or a conservative variant thereof. In an aspect of this embodiment, such a BoNT/A peptide is selected from one of the following amino acid sequences: 463-481 of SEQ ID NO:1 (N2), 519-537 of SEQ ID NO:1 (N6), 533-551 of SEQ ID NO:1 (N7), 547-565 of SEQ ID NO:1 (N8), 561-579 of SEQ ID NO:1 (N9), 589-607 of SEQ ID NO:1 (N11), 659-677 of SEQ ID NO:1 (N16), 743-761 of SEQ ID NO:l (N22), 785-803 of SEQ ID NO:1 (N25), 813-831 of SEQ ID NO:1 (N27), 827-845 of SEQ ID NO:1 (N28), 869-887 of SEQ ID NO:1 (C2), 883-901 of SEQ ID NO:1 (C3), 911-929 of SEQ ID NO:1 (C6), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1121-1139 of SEQ ID NO:1 (C20), 1135-1153 of SEQ ID NO:1 (C21), 1177-1195 of SEQ ID NO:1 (C24), 1261-1279 of SEQ ID NO:1 (C30) or 1275-1296 of SEQ ID NO:1 (C31), or a conservative variant thereof. In another aspect of this embodiment, such a BoNT/A peptide is selected from one of the following amino acid sequences: 463-481 of SEQ ID NO:1 (N2), 533-551 of SEQ ID NO:1 (N7), 547-565 of SEQ ID NO:1 (N8), 561-579 of SEQ ID NO:1 (N9), 659-677 of SEQ ID NO:1 (N16), 743-761 of SEQ ID NO:1 (N22), 785-803 of SEQ ID NO:1 (N25), 813-831 of SEQ ID NO:1 (N27), 883-901 of SEQ ID NO:1 (C3), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1121-1139 of SEQ ID NO:1 (C20) or 1177-1195 of SEQ ID NO:1 (C24) or 1275-1296 of SEQ ID NO: 1 (C31), or a conservative variant thereof. In another aspect of this embodiment, such a BoNT/A peptide is selected from one of the following amino acid sequences: 533-551 of SEQ ID NO: 1 (N7), 547-565 of SEQ ID NO: 1 (N8), 743-761 of SEQ ID NO: 1 (N22), 785-803 of SEQ ID NO: 1 (N25), 813-831 of SEQ ID NO: 1 (N27), 995-1013 of SEQ ID NO: 1 (C11); 1051-1069 of SEQ ID NO: 1 (C15), 1177-1195 of SEQ ID NO: 1 (C24), or 1275-1296 of SEQ ID NO: 1 (C31), or a conservative variant thereof.

In yet another aspect of this embodiment, a BoNT/A peptide has a length of at most 60 amino acids and consists of at least 5 contiguous amino acids selected from one of the following BoNT/A amino acid sequences: 463-481 of SEQ ID NO:1 (N2), 505-523 of SEQ ID NO:1 (N5), 519-537 of SEQ ID NO:1 (N6), 533-551 of SEQ ID NO:1 (N7), 603-621 of SEQ ID NO:1 (N12), 645-663 of SEQ ID NO:1 (N15), 659-677 of SEQ ID NO:1 (N16), 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1079-1097 of SEQ ID NO:1 (C17), 1107-1125 of SEQ ID NO:1 (C19), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28) or 1275-1296 of SEQ ID NO:1 (C31), or a conservative variant thereof. In yet another aspect of this embodiment, such a BoNT/A peptide is selected from one of the following amino acid sequences: 533-551 of SEQ ID NO:1 (N7), 659-677 of SEQ ID NO:1 (N16), 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1107-1125 of SEQ ID NO:1 (C19), 1163-1181 of SEQ ID NO:1 (C23), 1191-1209 of SEQ ID NO:1 (C25), 1233-1251 of SEQ ID NO:1 (C28) or 1275-1296 of SEQ ID NO:1 (C31), or a conservative variant thereof. In yet another aspect of this embodiment, such a BoNT/A peptide is selected from one of the following amino acid sequences: 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1079-1097 of SEQ ID NO:1 (C17), 1107-1125 of SEQ ID NO:1 (C19), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28) or 1275-1296 of SEQ ID NO:1 (C31), or a conservative variant thereof. In yet another aspect of this embodiment, such a BoNT/A peptide is selected from one of the following amino acid sequences: 729-747 of SEQ ID NO:1 (N21) 799-817 of SEQ ID NO:1 (N26), 1065-1083 of SEQ ID NO:1 (C16), 1163-1181 of SEQ ID NO:1 (C23) or 1275-1296 of SEQ ID NO:1 (C31), or a conservative variant thereof.

In yet another embodiment of the present invention, a BoNT/A peptide has a length of at most 60 amino acids and consists of at least 5 contiguous amino acids selected from one of the following BoNT/A amino acid sequences: 449-467 of SEQ ID NO:1(N1), 463-481 of SEQ ID NO:1 (N2), 491-509 of SEQ ID NO:1 (N4), 505-523 of SEQ ID NO:1 (N5), 519-537 of SEQ ID NO:1 (N6), 533-551 of SEQ ID NO:1 (N7), 547-565 of SEQ ID NO:1 (N8), 561-579 of SEQ ID NO:1 (N9), 575-593 of SEQ ID NO:1 (N10), 589-607 of SEQ ID NO:1 (N11), 631-649 of SEQ ID NO:1 (N14), 645-663 of SEQ ID NO:1 (N15), 659-677 of SEQ ID NO:1 (N16), 673-691 of SEQ ID NO:1 (N17), 701-719 of SEQ ID NO:1 (N19), 715-733 of SEQ ID NO:1 (N20), 729-747 of SEQ ID NO:1 (N21), 743-761 of SEQ ID NO:1 (N22), 757-775 of SEQ ID NO:1 (N23), 771-789 of SEQ ID NO:1 (N24), 785-803 of SEQ ID NO:1 (N25), 799-817 of SEQ ID NO:1 (N26), 813-831 of SEQ ID NO:1 (N27), 827-845 of SEQ ID NO:1 (N28), 869-887 of SEQ ID NO:1 (C2), 883-901 of SEQ ID NO:1 (C3), 911-929 of SEQ ID NO:1 (C5), 925-943 of SEQ ID NO:1 (C6), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1065-1083 of SEQ ID NO:1 (C16), 1079-1097 of SEQ ID NO:1 (C17), 1107-1125 of SEQ ID NO:1 (C19), 1121-1139 of SEQ ID NO:1 (C20), 1135-1153 of SEQ ID NO:1 (C21), 1149-1167 of SEQ ID NO:1 (C22), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28), 1247-1265 of SEQ ID NO:1 (C29), 1261-1279 of SEQ ID NO:1 (C30), or 1275-1296 of SEQ ID NO:1 (C31), or a non-conservative variant thereof. In an aspect of this embodiment, such a BoNT/A peptide is selected from one of the following amino acid sequences: 463-481 of SEQ ID NO:1 (N2), 519-537 of SEQ ID NO:1 (N6), 533-551 of SEQ ID NO:1 (N7), 547-565 of SEQ ID NO:1 (N8), 561-579 of SEQ ID NO:1 (N9), 589-607 of SEQ ID NO:1 (N11), 659-677 of SEQ ID NO:1 (N16), 743-761 of SEQ ID NO:1 (N22), 785-803 of SEQ ID NO:1 (N25), 813-831 of SEQ ID NO:1 (N27), 827-845 of SEQ ID NO:1 (N28), 869-887 of SEQ ID NO:1 (C2), 883-901 of SEQ ID NO:1 (C3), 911-929 of SEQ ID NO:1 (C6), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1121-1139 of SEQ ID NO:1 (C20), 1135-1153 of SEQ ID NO:1 (C21), 1177-1195 of SEQ ID NO:1 (C24), 1261-1279 of SEQ ID NO:1 (C30) or 1275-1296 of SEQ ID NO:1 (C31), or a non-conservative variant thereof. In another aspect of this embodiment, such a BoNT/A peptide is selected from one of the following amino acid sequences: 463-481 of SEQ ID NO:1 (N2), 533-551 of SEQ ID NO:1 (N7), 547-565 of SEQ ID NO:1 (N8), 561-579 of SEQ ID NO:1 (N9), 659-677 of SEQ ID NO:1 (N16), 743-761 of SEQ ID NO:1 (N22), 785-803 of SEQ ID NO:1 (N25), 813-831 of SEQ ID NO:1 (N27), 883-901 of SEQ ID NO:1 (C3), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1121-1139 of SEQ ID NO:1 (C20) or 1177-1195 of SEQ ID NO:1 (C24) or 1275-1296 of SEQ ID NO: 1 (C31), or a non-conservative variant thereof. In another aspect of this embodiment, such a BoNT/A peptide is selected from one of the following amino acid sequences: 533-551 of SEQ ID NO: 1 (N7), 547-565 of SEQ ID NO: 1 (N8), 743-761 of SEQ ID NO: 1 (N22), 785-803 of SEQ ID NO: 1 (N25), 813-831 of SEQ ID NO: 1 (N27), 995-1013 of SEQ ID NO: 1 (C11); 1051-1069 of SEQ ID NO: 1 (C15), 1177-1195 of SEQ ID NO: 1 (C24), or 1275-1296 of SEQ ID NO: 1 (C31), or a non-conservative variant thereof.

In yet another aspect of this embodiment, a BoNT/A peptide has a length of at most 60 amino acids and consists of at least 5 contiguous amino acids selected from one of the following BoNT/A amino acid sequences: 463-481 of SEQ ID NO:1 (N2), 505-523 of SEQ ID NO:1 (N5), 519-537 of SEQ ID NO:1 (N6), 533-551 of SEQ ID NO:1 (N7), 603-621 of SEQ ID NO:1 (N12), 645-663 of SEQ ID NO:1 (N15), 659-677 of SEQ ID NO:1 (N16), 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1079-1097 of SEQ ID NO:1 (C17), 1107-1125 of SEQ ID NO:1 (C19), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28) or 1275-1296 of SEQ ID NO:1 (C31), or a non-conservative variant thereof. In yet another aspect of this embodiment, such a BoNT/A peptide is selected from one of the following amino acid sequences: 533-551 of SEQ ID NO:1 (N7), 659-677 of SEQ ID NO:1 (N16), 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1107-1125 of SEQ ID NO:1 (C19), 1163-1181 of SEQ ID NO:1 (C23), 1191-1209 of SEQ ID NO:1 (C25), 1233-1251 of SEQ ID NO:1 (C28) or 1275-1296 of SEQ ID NO:1 (C31), or a non-conservative variant thereof. In yet another aspect of this embodiment, such a BoNT/A peptide is selected from one of the following amino acid sequences: 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1079-1097 of SEQ ID NO:1 (C17), 1107-1125 of SEQ ID NO:1 (C19), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28) or 1275-1296 of SEQ ID NO:1 (C31), or a non-conservative variant thereof. In yet another aspect of this embodiment, such a BoNT/A peptide is selected from one of the following amino acid sequences: 729-747 of SEQ ID NO:1 (N21) 799-817 of SEQ ID NO:1 (N26), 1065-1083 of SEQ ID NO:1 (C16), 1163-1181 of SEQ ID NO:1 (C23) or 1275-1296 of SEQ ID NO:1 (C31), or a non-conservative variant thereof.

In yet another embodiment of the present invention, a BoNT/A peptide has a length of at most 60 amino acids and consists of at least 5 contiguous amino acids selected from one of the following BoNT/A amino acid sequences: 449-467 of SEQ ID NO:1(N1), 463-481 of SEQ ID NO:1 (N2), 491-509 of SEQ ID NO:1 (N4), 505-523 of SEQ ID NO:1 (N5), 519-537 of SEQ ID NO:1 (N6), 533-551 of SEQ ID NO:1 (N7), 547-565 of SEQ ID NO:1 (N8), 561-579 of SEQ ID NO:1 (N9), 575-593 of SEQ ID NO:1 (N10), 589-607 of SEQ ID NO:1 (N11), 631-649 of SEQ ID NO:1 (N14), 645-663 of SEQ ID NO:1 (N15), 659-677 of SEQ ID NO:1 (N16), 673-691 of SEQ ID NO:1 (N17), 701-719 of SEQ ID NO:1 (N19), 715-733 of SEQ ID NO:1 (N20), 729-747 of SEQ ID NO:1 (N21), 743-761 of SEQ ID NO:1 (N22), 757-775 of SEQ ID NO:1 (N23), 771-789 of SEQ ID NO:1 (N24), 785-803 of SEQ ID NO:1 (N25), 799-817 of SEQ ID NO:1 (N26), 813-831 of SEQ ID NO:1 (N27), 827-845 of SEQ ID NO:1 (N28), 869-887 of SEQ ID NO:1 (C2), 883-901 of SEQ ID NO:1 (C3), 911-929 of SEQ ID NO:1 (C5), 925-943 of SEQ ID NO:1 (C6), 939-957 of SEQ ID NO:1 (C7), 967-985 of SEQ ID NO:1 (C9), 981-999 of SEQ ID NO:1 (C10), 995-1013 of SEQ ID NO:1 (C1), 1051-1069 of SEQ ID NO:1 (C15), 1065-1083 of SEQ ID NO:1 (C16), 1079-1097 of SEQ ID NO:1 (C17), 1107-1125 of SEQ ID NO:1 (C19), 1121-1139 of SEQ ID NO:1 (C20), 1135-1153 of SEQ ID NO:1 (C21), 1149-1167 of SEQ ID NO:1 (C22), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28), 1247-1265 of SEQ ID NO:1 (C29), 1261-1279 of SEQ ID NO:1 (C30), or 1275-1296 of SEQ ID NO:1 (C31), or an immunoreactive fragment thereof, with the proviso that the BoNT/A peptide is not SEQ ID NO:2. In an aspect of this embodiment, such a BoNT/A peptide is selected from one of the following amino acid sequences: 463-481 of SEQ ID NO:1 (N2), 519-537 of SEQ ID NO:1 (N6), 533-551 of SEQ ID NO:1 (N7), 547-565 of SEQ ID NO:1 (N8), 561-579 of SEQ ID NO:1 (N9), 589-607 of SEQ ID NO:1 (N11), 659-677 of SEQ ID NO:1 (N16), 743-761 of SEQ ID NO:1 (N22), 785-803 of SEQ ID NO:1 (N25), 813-831 of SEQ ID NO:1 (N27), 827-845 of SEQ ID NO:1 (N28), 869-887 of SEQ ID NO:1 (C2), 883-901 of SEQ ID NO:1 (C3), 911-929 of SEQ ID NO:1 (C6), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1121-1139 of SEQ ID NO:1 (C20), 1135-1153 of SEQ ID NO:1 (C21), 1177-1195 of SEQ ID NO:1 (C24), 1261-1279 of SEQ ID NO:1 (C30) or 1275-1296 of SEQ ID NO:1 (C31), or an immunoreactive fragment thereof, with the proviso that the BoNT/A peptide is not SEQ ID NO:2. In another aspect of this embodiment, such a BoNT/A peptide is selected from one of the following amino acid sequences: 463-481 of SEQ ID NO:1 (N2), 533-551 of SEQ ID NO:1 (N7), 547-565 of SEQ ID NO:1 (N8), 561-579 of SEQ ID NO:1 (N9), 659-677 of SEQ ID NO:1 (N16), 743-761 of SEQ ID NO:1 (N22), 785-803 of SEQ ID NO:1 (N25), 813-831 of SEQ ID NO:1 (N27), 883-901 of SEQ ID NO:1 (C3), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1121-1139 of SEQ ID NO:1 (C20) or 1177-1195 of SEQ ID NO:1 (C24) or 1275-1296 of SEQ ID NO: 1 (C31), or an immunoreactive fragment thereof, with the proviso that the BoNT/A peptide is not SEQ ID NO:2. In another aspect of this embodiment, such a BoNT/A peptide is selected from one of the following amino acid sequences: 533-551 of SEQ ID NO: 1 (N7), 547-565 of SEQ ID NO: 1 (N8), 743-761 of SEQ ID NO: 1 (N22), 785-803 of SEQ ID NO: 1 (N25), 813-831 of SEQ ID NO: 1 (N27), 995-1013 of SEQ ID NO: 1 (C11); 1051-1069 of SEQ ID NO: 1 (C15), 1177-1195 of SEQ ID NO: 1 (C24), or 1275-1296 of SEQ ID NO: 1 (C31), or an immunoreactive fragment thereof, with the proviso that the BoNT/A peptide is not SEQ ID NO:2.

In yet another aspect of this embodiment, a BoNT/A peptide has a length of at most 60 amino acids and consists of at least 5 contiguous amino acids selected from one of the following BoNT/A amino acid sequences: 463-481 of SEQ ID NO:1 (N2), 505-523 of SEQ ID NO:1 (N5), 519-537 of SEQ ID NO:1 (N6), 533-551 of SEQ ID NO:1 (N7), 603-621 of SEQ ID NO:1 (N12), 645-663 of SEQ ID NO:1 (N15), 659-677 of SEQ ID NO:1 (N16), 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1079-1097 of SEQ ID NO:1 (C17), 1107-1125 of SEQ ID NO:1 (C19), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28) or 1275-1296 of SEQ ID NO:1 (C31), or an immunoreactive fragment thereof, with the proviso that the BoNT/A peptide is not SEQ ID NO:2. In yet another aspect of this embodiment, such a BoNT/A peptide is selected from one of the following amino acid sequences: 533-551 of SEQ ID NO:1 (N7), 659-677 of SEQ ID NO:1 (N1 6), 701-719 of SEQ ID NO:1 (N1 9), 729-747 of SEQ ID NO:1 (N21), 757-775 of SEQ ID NO:1 (N23), 799-817 of SEQ ID NO:1 (N26), 1065-1083 of SEQ ID NO:1 (C16), 1107-1125 of SEQ ID NO:1 (C19), 1163-1181 of SEQ ID NO:1 (C23), 1191-1209 of SEQ ID NO:1 (C25), 1233-1251 of SEQ ID NO:1 (C28) or 1275-1296 of SEQ ID NO:1 (C31), or an immunoreactive fragment thereof, with the proviso that the BoNT/A peptide is not SEQ ID NO:2. In yet another aspect of this embodiment, such a BoNT/A peptide is selected from one of the following amino acid sequences: 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1079-1097 of SEQ ID NO:1 (C17), 1107-1125 of SEQ ID NO:1 (C19), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28) or 1275-1296 of SEQ ID NO:1 (C31), or an immunoreactive fragment thereof, with the proviso that the BoNT/A peptide is not SEQ ID NO:2. In yet another aspect of this embodiment, such a BoNT/A peptide is selected from one of the following amino acid sequences: 729-747 of SEQ ID NO:1 (N21) 799-817 of SEQ ID NO:1 (N26), 1065-1083 of SEQ ID NO:1 (C16), 1163-1181 of SEQ ID NO:1 (C23) or 1275-1296 of SEQ ID NO:1 (C31), or an immunoreactive fragment thereof, with the proviso that the BoNT/A peptide is not SEQ ID NO:2.

In is envisioned that a BoNT/A peptide disclosed in the present specification can have any of a variety of lengths from at least 5 amino acids to at most 60 amino acids. Therefore, aspects of this embodiment may include a BoNT/A peptide with at least, e.g., five amino acids, six amino acids, seven amino acids, eight amino acids, nine amino acids, ten amino acids, 11 amino acids, 12 amino acids, 13 amino acids, 14 amino acids, 15 amino acids, 16 amino acids, 17 amino acids, 18 amino acids, 19 amino acids, 20 amino acids, 25 amino acids, 30 amino acids, 35 amino acids, 40 amino acids, 45 amino acids, 50 amino acids, 55 amino acids or 60 amino acids. Other aspects of this embodiment may include a BoNT/A peptide with at least, e.g., five amino acids of SEQ ID NO:1, six amino acids of SEQ ID NO:1, seven amino acids of SEQ ID NO:1, eight amino acids of SEQ ID NO:1, nine amino acids of SEQ ID NO:1, ten amino acids of SEQ ID NO:1, 11 amino acids of SEQ ID NO:1, 12 amino acids of SEQ ID NO:1, 13 amino acids of SEQ ID NO:1, 14 amino acids of SEQ ID NO:1, 15 amino acids of SEQ ID NO:1, 16 amino acids of SEQ ID NO:1, 17 amino acids of SEQ ID NO:1, 18 amino acids of SEQ ID NO:1, 19 amino acids of SEQ ID NO:1, 20 amino acids of SEQ ID NO:1, 25 amino acids of SEQ ID NO:1, 30 amino acids of SEQ ID NO:1, 35 amino acids of SEQ ID NO:1, 40 amino acids of SEQ ID NO:1, 45 amino acids of SEQ ID NO:1, 50 amino acids of SEQ ID NO:1, 55 amino acids or 60 amino acids of SEQ ID NO:1. In further embodiments, such a BoNT/A peptide of the invention may include a BoNT/A peptide with at least, e.g., five amino acids, six amino acids, seven amino acids, eight amino acids, nine amino acids, ten amino acids, 11 amino acids, 12 amino acids, 13 amino acids, 14 amino acids, 15 amino acids, 16 amino acids, 17 amino acids, 18 amino acids, 19 amino acids, 20 amino acids, 25 amino acids, 30 amino acids, 35 amino acids, 40 amino acids, 45 amino acids, 50 amino acids, 55 amino acids or 60 amino acids and consist of at least 5 contiguous amino acids selected from one of the following BoNT/A amino acid sequences: 449-467 of SEQ ID NO:1(N1), 463-481 of SEQ ID NO:1 (N2), 491-509 of SEQ ID NO:1 (N4), 505-523 of SEQ ID NO:1 (N5), 519-537 of SEQ ID NO:1 (N6), 533-551 of SEQ ID NO:1 (N7), 547-565 of SEQ ID NO:1 (N8), 561-579 of SEQ ID NO:1 (N9), 575-593 of SEQ ID NO:1 (N10), 589-607 of SEQ ID NO:1 (N11), 631-649 of SEQ ID NO:1 (N14), 645-663 of SEQ ID NO:1 (N15), 659-677 of SEQ ID NO:1 (N16), 673-691 of SEQ ID NO:1 (N17), 701-719 of SEQ ID NO:1 (N19), 715-733 of SEQ ID NO:1 (N20), 729-747 of SEQ ID NO:1 (N21), 743-761 of SEQ ID NO:1 (N22), 757-775 of SEQ ID NO:1 (N23), 771-789 of SEQ ID NO:1 (N24), 785-803 of SEQ ID NO:1 (N25), 799-817 of SEQ ID NO:1 (N26), 813-831 of SEQ ID NO:1 (N27); 827-845 of SEQ ID NO:1 (N28), 869-887 of SEQ ID NO:1 (C2), 883-901 of SEQ ID NO:1 (C3), 911-929 of SEQ ID NO:1 (C5), 925-943 of SEQ ID NO:1 (C6), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1065-1083 of SEQ ID NO:1 (C16), 1079-1097 of SEQ ID NO:1 (C17), 1107-1125 of SEQ ID NO:1 (C19), 1121-1139 of SEQ ID NO:1 (C20), 1135-1153 of SEQ ID NO:1 (C21), 1149-1167 of SEQ ID NO:1 (C22), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28), 1247-1265 of SEQ ID NO:1 (C29), 1261-1279 of SEQ ID NO:1 (C30) or 1275-1296 of SEQ ID NO:1 (C31), or a conservative variant, a non-conservative variant, or immunoreactive fragment thereof, with the proviso that the BoNT/A peptide is not SEQ ID NO:2.

In another embodiment of the present invention, a BoNT/A peptide composition can comprise one BoNT/A peptide disclosed in the present specification. In another embodiment of the present invention, a BoNT/A peptide composition can comprise a plurality of BoNT/A peptides disclosed in the present specification. Thus, aspects of this embodiment can include one or more BoNT/A peptides, two or more BoNT/A peptides, three or more BoNT/A peptides, four or more BoNT/A peptides, five or more BoNT/A peptides, six or more BoNT/A peptides, seven or more BoNT/A peptides, eight or more BoNT/A peptides, nine or more BoNT/A peptides, ten or more BoNT/A peptides, 15 or more BoNT/A peptides, 20 or more BoNT/A peptides, 25 or more BoNT/A peptides or 30 or more BoNT/A peptides. In other aspects of this embodiment can include one or more BoNT/A conservative variants, two or more BoNT/A conservative variants, three or more BoNT/A conservative variants, four or more BoNT/A conservative variants, five or more BoNT/A conservative variants, six or more BoNT/A conservative variants, seven or more BoNT/A conservative variants, eight or more BoNT/A conservative variants, nine or more BoNT/A conservative variants, ten or more BoNT/A conservative variants, 15 or more BoNT/A conservative variants, 20 or more BoNT/A conservative variants, 25 or more BoNT/A conservative variants or 30 or more BoNT/A conservative variants. In further aspects of this embodiment can include one or more BoNT/A non-conservative variants, two or more BoNT/A non-conservative variants, three or more BoNT/A non-conservative variants, four or more BoNT/A non-conservative variants, five or more BoNT/A non-conservative variants, six or more BoNT/A non-conservative variants, seven or more BoNT/A non-conservative variants, eight or more BoNT/A non-conservative variants, nine or more BoNT/A non-conservative variants, ten or more BoNT/A non-conservative variants, 15 or more BoNT/A non-conservative variants, 20 or more BoNT/A non-conservative variants, 25 or more BoNT/A non-conservative variants or 30 or more BoNT/A non-conservative variants. In still other aspects of this embodiment can include one or more BoNT/A immunoreactive fragments, two or more BoNT/A immunoreactive fragments, three or more BoNT/A immunoreactive fragments, four or more BoNT/A immunoreactive fragments, five or more BoNT/A immunoreactive fragments, six or more BoNT/A immunoreactive fragments, seven or more BoNT/A immunoreactive fragments, eight or more BoNT/A immunoreactive fragments, nine or more BoNT/A peptides, ten or more BoNT/A immunoreactive fragments, 15 or more BoNT/A immunoreactive fragments, 20 or more BoNT/A immunoreactive fragments, 25 or more BoNT/A immunoreactive fragments or 30 or more BoNT/A immunoreactive fragments. BoNT/A peptides disclosed in the present specification can be selected, for example, depending on immunological factors, such as potency of the peptide in inducing an immune response, and technical factors, such as chemical synthesis yields. It is also understood that the two or more BoNT/A peptides can be provided separately or as part of a compound molecule such as a chimeric peptide or heterologous protein.

In an aspect of this embodiment, a BoNT/A peptide composition comprises two or more of the following amino acid sequences: 533-551 of SEQ ID NO:1 (N7), 547-565 of SEQ ID NO:1 (N8), 743-761 of SEQ ID NO:1 (N22), 785-803 of SEQ ID NO:1 (N25), 813-831 of SEQ ID NO:1 (N27), 995-1013 of SEQ ID NO:1 (C11); 1051-1069 of SEQ ID NO:1 (C15), 1177-1195 of SEQ ID NO:1 (C24), and 1275-1296 of SEQ ID NO:1 (C31), or a conservative variant, a non-conservative variant or an immunoreactive fragment thereof, with the proviso that the BoNT/A peptide is not SEQ ID NO:2. In another aspect of this embodiment, one of the selected amino acid sequence is 533-551 of SEQ ID NO:1 (N8) or a conservative variant, a non-conservative variant or an immunoreactive fragment thereof, with the proviso that the BoNT/A peptide is not SEQ ID NO:2. In yet another aspect of this embodiment, the following two amino acid sequences are selected: 533-551 of SEQ ID NO:1 (N8) and 981-999 of SEQ ID NO:1 (C10), or a conservative variant, a non-conservative variant or an immunoreactive fragment thereof, with the proviso that the BoNT/A peptide is not SEQ ID NO:2. In yet another aspect of this embodiment, the following three amino acid sequences are selected: 533-551 of SEQ ID NO:1 (N8), 981-999 of SEQ ID NO:1 (C10) and 1051-1069 of SEQ ID NO:1 (C15), or a conservative variant, a non-conservative variant or an immunoreactive fragment thereof, with the proviso that the BoNT/A peptide is not SEQ ID NO:2. In a further aspect of this embodiment, one of the amino acid sequences selected is 785-803 of SEQ ID NO:1 (N25) or a conservative variant, a non-conservative variant or an immunoreactive fragment thereof, with the proviso that the BoNT/A peptide is not SEQ ID NO:2. In a still further aspect of this embodiment, the following two amino acid sequences are selected: 785-803 of SEQ ID NO:1 (N25) and 981-999 of SEQ ID NO:1 (C10), or a conservative variant, a non-conservative variant or an immunoreactive fragment thereof, with the proviso that the BoNT/A peptide is not SEQ ID NO:2. In a still further aspect of this embodiment, the following three amino acid sequences are selected: 785-803 of SEQ ID NO:1 (N25), 981-999 of SEQ ID NO:1 (C10) and 1051-1069 of SEQ ID NO:1 (C15), or a conservative variant, a non-conservative variant or an immunoreactive fragment thereof, with the proviso that the BoNT/A peptide is not SEQ ID NO:2. In an additional aspect of this embodiment, one of the amino acid sequences selected is 813-831 of SEQ ID NO:1 (N27) or a conservative variant, a non-conservative variant or an immunoreactive fragment thereof, with the proviso that the BoNT/A peptide is not SEQ ID NO:2. In another additional aspect of this embodiment, the following two amino acid sequences are selected: 813-831 of SEQ ID NO:1 (N27) and 981-999 of SEQ ID NO:1 (C10), or a conservative variant, a non-conservative variant or an immunoreactive fragment thereof, with the proviso that the BoNT/A peptide is not SEQ ID NO:2. In another additional aspect of this embodiment, the following three amino acid sequences are selected: 813-831 of SEQ ID NO:1 (N27), 981-999 of SEQ ID NO:1 (C10) and 1051-1069 of SEQ ID NO:1 (C15), or a conservative variant, a non-conservative variant or an immunoreactive fragment thereof, with the proviso that the BoNT/A peptide is not SEQ ID NO:2.

In an aspect of this embodiment, a BoNT/A peptide composition comprises two or more of the following amino acid sequences: 659-677 of SEQ ID NO:1 (N16), 729-747 of SEQ ID NO:1 (N21), 799-817 of SEQ ID NO:1 (N26), 1065-1083 of SEQ ID NO:1 (C16), 1163-1181 of SEQ ID NO:1 (C23), and 1275-1296 of SEQ ID NO:1 (C31), or a conservative variant, a non-conservative variant or an immunoreactive fragment thereof, with the proviso that the BoNT/A peptide is not SEQ ID NO:2. In another aspect of this embodiment, one of the amino acid sequences selected is 1065-1083 of SEQ ID NO:1 (C16) or a conservative variant, a non-conservative variant or an immunoreactive fragment thereof, with the proviso that the BoNT/A peptide is not SEQ ID NO:2. In yet another aspect of this embodiment, the following two amino acid sequences are selected: 1065-1083 of SEQ ID NO:1 (C16) and 1163-1181 of SEQ ID NO:1 (C23), or a conservative variant, a non-conservative variant or an immunoreactive fragment thereof, with the proviso that the BoNT/A peptide is not SEQ ID NO:2. In yet another aspect of this embodiment, the following three amino acid sequences are selected: 1065-1083 of SEQ ID NO:1 (C16), 1163-1181 of SEQ ID NO:1 (C23) and 1275-1296 of SEQ ID NO:1 (C31), or a conservative variant, a non-conservative variant or an immunoreactive fragment thereof, with the proviso that the BoNT/A peptide is not SEQ ID NO:2. In a further aspect of this embodiment, one of the amino acid sequences selected is 799-817 of SEQ ID NO:1 (N26) or a conservative variant, a non-conservative variant or an immunoreactive fragment thereof, with the proviso that the BoNT/A peptide is not SEQ ID NO:2. In a still further aspect of this embodiment, the following two amino acid sequences are selected: 799-817 of SEQ ID NO:1 (N26) and 1065-1083 of SEQ ID NO:1 (C16), or a conservative variant, a non-conservative variant or an immunoreactive fragment thereof, with the proviso that the BoNT/A peptide is not SEQ ID NO:2. In a still further aspect of this embodiment, the following three amino acid sequences are selected: 799-817 of SEQ ID NO:1 (N26), 1065-1083 of SEQ ID NO:1 (C16) and 1163-1181 of SEQ ID NO:1 (C23), or a conservative variant, a non-conservative variant or an immunoreactive fragment thereof, with the proviso that the BoNT/A peptide is not SEQ ID NO:2. In an additional aspect of this embodiment, one of the amino acid sequences selected is 729-747 of SEQ ID NO:1 (N21) or a conservative variant, a non-conservative variant or an immunoreactive fragment thereof, with the proviso that the BoNT/A peptide is not SEQ ID NO:2. In another additional aspect of this embodiment, the following two amino acid sequences are selected: 729-747 of SEQ ID NO:1 (N21) and 1065-1083 of SEQ ID NO:1 (C16), or a conservative variant, a non-conservative variant or an immunoreactive fragment thereof, with the proviso that the BoNT/A peptide is not SEQ ID NO:2. In another additional aspect of this embodiment, the following three amino acid sequences are selected: 729-747 of SEQ ID NO:1 (N21), 1065-1083 of SEQ ID NO:1 (C16) and 1163-1181 of SEQ ID NO:1 (C23), or a conservative variant, a non-conservative variant or an immunoreactive fragment thereof, with the proviso that the BoNT/A peptide is not SEQ ID NO:2.

It is also envisioned that any and all combinations of BoNT/A peptides disclosed in the specification, including, e.g., BoNT/A peptides of SEQ ID NO:1, BoNT/A conservative variants, BoNT/A non-conservative variants and BoNT/A immunoreactive fragments. Thus, aspects of this embodiment include one or more BoNT/A peptides comprising one or more BoNT/A peptides of SEQ ID NO: 1 and one or more BoNT/A conservative variants; one or more BoNT/A peptides of SEQ ID NO: 1 and one or more BoNT/A non-conservative variants; one or more BoNT/A peptides of SEQ ID NO: 1 and one or more BoNT/A immunoreactive fragments; one or more BoNT/A conservative variants and one or more BoNT/A non-conservative variants; one or more BoNT/A conservative variants and one or more BoNT/A immunoreactive fragments; one or more BoNT/A non-conservative variants and one or more BoNT/A immunoreactive fragments; one or more BoNT/A peptides of SEQ ID NO: 1, one or more BoNT/A conservative variants and one or more BoNT/A non-conservative variants; one or more BoNT/A peptides of SEQ ID NO: 1, one or more BoNT/A conservative variants and one or more BoNT/A immunoreactive fragments; one or more BoNT/A peptides of SEQ ID NO: 1, one or more BoNT/A non-conservative variants and one or more BoNT/A immunoreactive fragments; one or more BoNT/A conservative variants, one or more BoNT/A non-conservative variants and one or more BoNT/A immunoreactive fragments; or one or more BoNT/A peptides of SEQ ID NO: 1, one or more BoNT/A conservative variants, one or more BoNT/A non-conservative variants and one or more BoNT/A immunoreactive fragments.

A. BoNT/A Peptides

As used herein, the term “peptide” means two or more amino acids covalently bonded together. The term “BoNT/A peptide,” as used herein, means a peptide having a length of at least five amino acids and at most 60 amino acids and can function in substantially the same manner as the corresponding BoNT/A peptide of SEQ ID NO:1 and can be substituted for the corresponding BoNT/A peptide of SEQ ID NO:1 in any aspect of the present invention. Thus, a BoNT/A peptide can be, for example, a peptide of at least five amino acids and at most 60 amino acids having an amino acid sequence corresponding to a portion of the naturally occurring BoNT/A sequence SEQ ID NO:1, such a peptide having one or more conservative or non-conservative substitutions relative to a portion of SEQ ID NO:1, a conservative variant or non-conservative variant of a portion of a BoNT/A peptide of SEQ ID NO:1, or an immunoreactive fragment. The term “BoNT/A peptide” encompasses “non-conservative variants,” “conservative variants” and “immunoreactive fragments,” each of which is described further below. Specifically excluded from the definition of a BoNT/A peptide is the 57-mer SEQ ID NO:2 described in Toru Kubota et al., Epitope Regions in the Heavy Chain of Clostridium Botulinum Type E Neurotoxin Recognized by Monoclonal Antibodies, 63(4) APPL. ENVIRON. MICROBIOL. 1214-1218 (1997). In one embodiment, a BoNT/A peptide is not SEQ ID NO:10 or a fragment thereof.

B. BoNT/A Conservative Variants

As used herein in reference to BoNT/A, the term “conservative variant” means a BoNT/A peptide that has been altered from the BoNT/A peptide of SEQ ID NO: 1 in which a first amino acid from the BoNT/A peptide of SEQ ID NO: 1 is substituted by another amino acid or an amino acid analog that has at least one property similar to that of the first amino acid. Examples of properties include, without limitation, similar size, topography, charge, hydrophobicity, hydrophilicity, lipophilicity covalant-bonding capacity, hydrogen-bonding capacity, a physicochemically property, of the like, or any combination thereof. A conservative BoNT/A variant can function in substantially the same manner as the BoNT/A peptide of SEQ ID NO: 1 and can be substituted for the BoNT/A peptide of SEQ ID NO: 1 in any aspect of the present invention. A conservative BoNT/A variant may substitute one or more amino acids, two or more amino acids, three or more amino acids, four or more amino acids, five or more amino acids, ten or more amino acids, 20 or more amino acids, 30 or more amino acids, 40 or more amino acids, 50 or more amino acids of the BoNT/A peptide of SEQ ID NO: 1, or a portion thereof, and that such variants can include naturally and non-naturally occurring amino acid analogs as described further below.

As a non-limiting example, a conservative variant can be a sequence in which a first uncharged polar amino acid is conservatively substituted with a second (non-identical) uncharged polar amino acid such as cysteine, serine, threonine, tyrosine, glycine, glutamine or asparagine or an analog thereof. A conservative variant also can be, for example, a sequence in which a first basic amino acid is conservatively substituted with a second basic amino acid such as arginine, lysine, histidine, 5-hydroxylysine, N-methyllysine or an analog thereof. Similarly, a conservative variant can be, for example, a sequence in which a first hydrophobic amino acid is conservatively substituted with a second hydrophobic amino acid such as alanine, valine, leucine, isoleucine, proline, methionine, phenylalanine or tryptophan or an analog thereof. In the same way, a conservative variant can be, for example, a sequence in which a first acidic amino acid is conservatively substituted with a second acidic amino acid such as aspartic acid or glutamic acid or an analog thereof; a sequence in which an aromatic amino acid such as phenylalanine is conservatively substituted with a second aromatic amino acid or amino acid analog, for example, tyrosine; or a sequence in which a first relatively small amino acid such as alanine is substituted with a second relatively small, amino acid or amino acid analog such as glycine or valine or an analog thereof.

As a non-limiting example, conservative variants of BoNT/A peptides include conservative variants of a BoNT/A peptide having residues 445-471 of SEQ ID NO:1; such conservative variants can have, for example, an arginine for lysine substitution at position 456 and an isoleucine for leucine substitution at position 462. Additional conservative variants include conservative variants of the BoNT/A peptide having residues 487-513 of SEQ ID NO:1; such conservative variants can have, for example, a glutamic acid for aspartic acid substitution at position 497; an asparagine for glutamine substitution at position 500; and a phenylalanine for tyrosine substitution at position 502.

C. BoNT/A Non-Conservative Variants

As used herein in reference to BoNT/A, the term “non-conservative BoNT/A variant,” means a BoNT/A peptide that has been altered from the BoNT/A peptide of SEQ ID NO: 1 in which 1) a first amino acid is deleted from the BoNT/A peptide of SEQ ID NO: 1; 2) a second amino acid is added to the BoNT/A peptide of SEQ ID NO: 1; or 3) a first amino acid of the BoNT/A peptide of SEQ ID NO: 1 is substituted by a second amino acid or amino acid analog that does not share any property similar to that of the first amino acid. A non-conservative BoNT/A variant can function in substantially the same manner as the BoNT/A peptide of SEQ ID NO: 1 and can be substituted for the BoNT/A peptide of SEQ ID NO: 1 in any aspect of the present invention. A non-conservative BoNT/A variant can delete one or more amino acids, two or more amino acids, three or more amino acids, four or more amino acids, five or more amino acids, and ten or more amino acids from the BoNT/A peptide of SEQ ID NO: 1, or a portion thereof. A non-conservative BoNT/A variant can add one or more amino acids, two or more amino acids, three or more amino acids, four or more amino acids, five or more amino acids, and ten or more amino acids to the BoNT/A peptide of SEQ ID NO: 1, or a portion thereof. A non-conservative BoNT/A variant can substitute at least 10 contiguous amino acids, at least 15 contiguous amino acids, at least 20 contiguous amino acids, or at least 25 contiguous amino acids from the BoNT/A peptide of SEQ ID NO: 1, or a portion thereof, that possess at least 50% amino acid identity, 65% amino acid identity, 75% amino acid identity, 85% amino acid identity or 95% amino acid identity to the BoNT/A peptide of SEQ ID NO: 1, or a portion thereof, and is capable of selective antibody binding raised against the BoNT/A peptide of SEQ ID NO: 1, or a portion thereof.

D. BoNT/A Immunoreactive Fragments

As used herein in reference to BoNT/A, the term “immunoreactive fragment” means a BoNT/A peptide capable of selectively binding an anti-BoNT/A antibody, with the proviso that the BoNT/A peptide is not SEQ ID NO:2. As used herein, the term “selectively” means having a unique effect or influence or reacting in only one way or with only one thing. An immunoreactive BoNT/A fragment can function in substantially the same manner as the BoNT/A peptide of SEQ ID NO: 1 and can be substituted for the BoNT/A peptide of SEQ ID NO: 1 in any aspect of the present invention. An immunoreactive fragment can be capable of selective antibody binding to anti-BoNT/A antibodies from one or more species. An immunoreactive fragment of a BoNT/A peptide generally has from about six amino acids to 60 amino acids. An immunoreactive fragment of a BoNT/A peptide can havea length of at least, e,g., 5, 6, 7, 8, 9, 10, 12, 15, 18, 20 or 25 amino acids. An immunoreactive fragment of a BoNT/A peptide also can havea length of at most, e.g., 8, 9, 10, 12, 15, 18, 20, 25, 30 or 35 amino acids. In particular embodiments, an immunoreactive fragment of a BoNT/A peptide has from five to sixty amino acids, from five to fifty amino acids, from eight to fifty amino acids, from ten to fifty amino acids, from five to twenty amino acids, from eight to twenty amino acids, from ten to twenty amino acids, from twelve to twenty amino acids or from fifteen to twenty amino acids. An immunoreactive fragment can have any number of conservative, non-conservative, analog or mimetic substitutions, and the like, as disclosed in the present specification.

An immunoreactive fragment can be identified using any of a variety of routine assays for detecting peptide antigen-antibody complexes, the presence of which is an indicator of selective binding. Such assays include, without limitation, enzyme-linked immunosorbent assays, radioimmunoassays, western blotting, enzyme immunoassays, fluorescence immunoassays, luminescent immunoassays and the like and generally are equivalent to the radioimmunoassay disclosed herein in Example I. Methods for detecting a complex between a peptide and an antibody, and thereby determining if the peptide is an “immunoreactive fragment” are well known to those skilled in the art and are described, for example, in ANTIBODIES: A LABORATORY MANUAL (Edward Harlow & David Lane, eds., Cold Spring Harbor Laboratory Press, 2^{nd} ed. 1998a); and USING ANTIBODIES: A LABORATORY MANUAL: PORTABLE PROTOCOL No. I (Edward Harlow & David Lane, Cold Spring Harbor Laboratory Press, 1998b), which are hereby incorporated by reference in their entirety.

As used herein, the term “amino acid” means both naturally occurring and non-naturally occurring amino acids as well as amino acid analogs and mimetics, and includes, but is not limited to, alanyl, valinyl, leucinyl, isoleucinyl, prolinyl, phenylalaninyl, tryptophanyl, methioninyl, glycinyl, serinyl, threoninyl, cysteinyl, tyrosinyl, asparaginyl, glutaminyl, aspartoyl, glutaoyl, lysinyl, argininyl, and histidinyl. As such, a BoNT/A peptide such as, e.g., a native peptide, a conservative variant, a non-conservative variant, or an immunoreactive fragment, can contain one or more non-amide linkage substitutions between amino acids, one or more naturally occurring amino acid substitutions, one or more non-naturally occurring amino acid substitutions, one or more amino acid analog substitutions, or one or more mimetic substitutions. As used herein in reference to BoNT/A, the term “naturally occurring amino acid substitution” means a BoNT/A peptide that has been altered from the BoNT/A peptide of SEQ ID NO: 1 in which a first amino acid from the BoNT/A peptide of SEQ ID NO: 1 is substituted by a naturally occurring amino acid that has at least one property similar to that of the first amino acid. Examples of naturally occurring amino acids, include, without limitation, Naturally occurring amino acids include the 20 (L)-amino acids utilized during protein biosynthesis as well as others such as, without limitation, 4-hydroxyproline, hydroxylysine, desmosine, isodesmosine, homocysteine, citrulline and ornithine.

As used herein in reference to BoNT/A, the term “non-naturally occurring amino acid substitution” means a BoNT/A peptide that has been altered from the BoNT/A peptide of SEQ ID NO: 1 in which a first amino acid from the BoNT/A peptide of SEQ ID NO: 1 is substituted by a non-naturally occurring amino acid that has at least one property similar to that of the first amino acid. Examples of non-naturally occurring amino acids, include, without limitation, (D)-amino acids, norleucine, norvaline, p-fluorophenylalanine, ethionine and the like.

As used herein in reference to BoNT/A, the term “amino acid analog substitution” means a BoNT/A peptide that has been altered from the BoNT/A peptide of SEQ ID NO: 1 in which a first amino acid from the BoNT/A peptide of SEQ ID NO: 1 is substituted by a modified natural or non-natural amino acid that has at least one property similar to that of the first amino acid. Examples of modifications to either a naturally and non-naturally occurring amino acids, include, without limitation, substitution or replacement of chemical groups or moieties on the amino acid or by derivitization of the amino acid. A BoNT/A amino acid analog can function in substantially the same manner as the BoNT/A peptide of SEQ ID NO: 1 and can be substituted for the BoNT/A peptide of SEQ ID NO: 1 in any aspect of the present invention. A BoNT/A amino acid analog may substitute one or more amino acids, two or more amino acids, three or more amino acids, four or more amino acids, five or more amino acids, ten or more amino acids, 20 or more amino acids, 30 or more amino acids, 40 or more amino acids, 50 or more amino acids from the BoNT/A peptide of SEQ ID NO: 1, or a portion thereof.

As used herein in reference to BoNT/A, the term “mimetic substitution” means a BoNT/A peptide that has been altered from the BoNT/A peptide of SEQ ID NO: 1 in which a first amino acid from the BoNT/A peptide of SEQ ID NO: 1 is substituted by a non-natural structure that has at least one property similar to that of the first amino acid. Examples of mimetic properties include, without limitation, topography of a peptide primary structural element, functionality of a peptide primary structural element, topology of a peptide secondary structural element, functionality of a peptide secondary structural element, of the like, or any combination thereof. A BoNT/A mimetic can function in substantially the same manner as the BoNT/A peptide of SEQ ID NO: 1 and can be substituted for the BoNT/A peptide of SEQ ID NO: 1 in any aspect of the present invention. A BoNT/A mimetic may substitute one or more amino acids, two or more amino acids, three or more amino acids, four or more amino acids, five or more amino acids, ten or more amino acids, 20 or more amino acids, 30 or more amino acids, 40 or more amino acids, 50 or more amino acids from the BoNT/A peptide of SEQ ID NO: 1, or a portion thereof. As an example, an organic structure that mimics arginine can have a positive charge moiety located in similar molecular space and having the same degree of mobility as the e-amino group of the side chain of the naturally occurring arginine amino acid.

Non-limiting examples of specific protocols for making and using naturally occurring amino acids, non-naturally occurring amino acids, amino acid analogs and mimetics are described in, e.g., John Jones, AMINO ACID PEPTIDE SYNTHESIS (Oxford University Press, 2^{nd} ed., 2002); Roberts and Vellaccio, p. 341 (THE PEPTIDES: ANALYSIS, SYNTHESIS, BIOLOGY Vol. 5, Erhard Gross & Johannes Meinhofer, eds., Academic Press, Inc., 1983); Mark J. Suto et al., Cytokine Restraining Agents, U.S. Pat. No. 5,420,109 (May 30, 1995); Chapter 7 of Bodanzsky, PRINCIPLES OF PEPTIDE SYNTHESIS (Springer-Verlag, 2^{nd} ed.1993); Stewart and Young SOLID PHASE PEPTIDE SYNTHESIS, Pierce Chemical Co., 2^{nd} ed. 1984); FMOC SOLID PHASE PEPTIDE SYNTHESIS: A PRACTICAL APPROACH (Weng C. Chan & Peter D. White eds., Oxford University Press, 2000); Amy S. Ripka & Daniel H. Rich, Peptidomimetic design, 2(4) CURR. OPIN. CHEM. BIOL. 441-452 (1998); and M. Angels Estiarte & Daniel H. Rich, Peptidomimetics for Drug Design, 803-861 (BURGER'S MEDICINAL CHEMISTRY AND DRUG DISCOVERY Vol. 1 PRINCIPLE AND PRACTICE, Donald J. Abraham ed., Wiley-Interscience, 6^{th} ed 2003), which are hereby incorporated by reference. One skilled in the art understands that these and other well known amino acid analogs and mimetics can be useful in the BoNT/A peptides of the invention.

A BoNT/A peptide disclosed in the present specification, such as, e.g., native peptide, a conservative variant, a non-conservative variant, or an immunoreactive fragment, can be fused to a heterologous protein. As used herein, the term “heterologous protein” means a protein derived from a source other than the gene encoding the BoNT/A peptide of the invention, operationally linked to a BoNT/A peptide disclosed in the present specification, to form a chimeric BoNT/A protein. Such a chimeric BoNT/A protein of the invention can have a variety of lengths including, but not limited to, a length of at most 100 residues, at most 200 residues, at most 300 residues, at most 400 residues, at most 500 residues, at most 800 residues or at most 1000 residues. Non-limiting examples of chimeric BoNT/A proteins include fusions of BoNT/A peptides with immunogenic polypeptides, such as flagellin and cholera enterotoxin; fusions of BoNT/A peptides with immunomodulatory polypeptides, such as IL-2 and B7-1; fusions of BoNT/A peptides with tolerogenic polypeptides, such as another BoNT/A peptide and an antibody selectively reactive with interleukin-12; and fusions of BoNT/A peptides with synthetic sequences.

II. BoNT/A Tolerogenic Compositions

Tolerance is an active antigen-dependent process that occurs in a human or other mammal in response to the antigen that results from a previous exposure to the same antigen. Generally speaking, the production of antibodies by an immune response occurs by a two-step process. Initially, B Imphocytes migrating through the lymphoid tissue are exposed to an antigen whereby these cells become partially activated. Subsequently, if a partially activated B cell encounters a T cell that has also been activated by the same antigen, antibodies against that antigen are produced. If the B cell does not receive the appropriate signal from the corresponding T cell, it will become inactive or die. Immune tolerance is a natural mechanism that eliminates development of B cells that target “self,” rather than foreign antigens. Therapeutic methods using tolerogizing compositions can exploit this immune tolerance system. For example, binding of a tolerogizing composition to a specific B cell is thought to stop production of pathogenic antibodies by causing the inactivatation or death of these pathogenic B cells. In general, a tolerogizing composition that can be used to tolerize B cells in an antigen-specific manner lacks the ability to activate T cells, but retains the ability to bind immune B cells. Therefore, a human or other mammal suffering from an immune response to a particular antigen can be treated with a tolerizing composition and become “tolerized” to that particular antigen.

Thus, the present invention further provides a tolerogizing composition comprising a tolerizing agent operationally linked to a BoNT/A peptide disclosed in the present specification useful, without limitation, for inducing specific immunological non-reactivity (tolerance) to a botulinum toxin antigen. In one embodiment of the present invention, a tolerogizing composition comprises a tolerizing agent operationally linked to a BoNT/A peptide having a length of at most 60 amino acids and consisting of at least 5 contiguous amino acids selected from one of the following BoNT/A amino acid sequences: 449-467 of SEQ ID NO:1(N1), 463-481 of SEQ ID NO:1 (N2), 491-509 of SEQ ID NO:1 (N4), 505-523 of SEQ ID NO:1 (N5), 519-537 of SEQ ID NO:1 (N6), 533-551 of SEQ ID NO:1 (N7), 547-565 of SEQ ID NO:1 (N8), 561-579 of SEQ ID NO:1 (N9), 575-593 of SEQ ID NO:1 (N10), 589-607 of SEQ ID NO:1 (N11), 631-649 of SEQ ID NO:1 (N14), 645-663 of SEQ ID NO:1 (N15), 659-677 of SEQ ID NO:1 (N16), 673-691 of SEQ ID NO:1 (N17), 701-719 of SEQ ID NO:1 (N19), 715-733 of SEQ ID NO:1 (N20), 729-747 of SEQ ID NO:1 (N21), 743-761 of SEQ ID NO:1 (N22), 757-775 of SEQ ID NO:1 (N23), 771-789 of SEQ ID NO:1 (N24), 785-803 of SEQ ID NO:1 (N25), 799-817 of SEQ ID NO:1 (N26), 813-831 of SEQ ID NO:1 (N27), 827-845 of SEQ ID NO:1 (N28), 869-887 of SEQ ID NO:1 (C2), 883-901 of SEQ ID NO:1 (C3), 911-929 of SEQ ID NO:1 (C5), 925-943 of SEQ ID NO:1 (C6), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1065-1083 of SEQ ID NO:1 (C16), 1079-1097 of SEQ ID NO:1 (C17), 1107-1125 of SEQ ID NO:1 (C19), 1121-1139 of SEQ ID NO:1 (C20), 1135-1153 of SEQ ID NO:1 (C21), 1149-1167 of SEQ ID NO:1 (C22), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28), 1247-1265 of SEQ ID NO:1 (C29), 1261-1279 of SEQ ID NO:1 (C30), or 1275-1296 of SEQ ID NO:1 (C31), or a conservative variant or immunoreactive fragment thereof, with the proviso that the BoNT/A peptide is not SEQ ID NO:2.

In another embodiment of the present invention, a tolerogizing composition comprises a tolerizing agent operationally linked to a BoNT/A peptide having a length of at most 60 amino acids and consisting of at least 5 contiguous amino acids selected from one of the following BoNT/A amino acid sequences: 449-467 of SEQ ID NO:1(N1), 463-481 of SEQ ID NO:1 (N2), 491-509 of SEQ ID NO:1 (N4), 505-523 of SEQ ID NO:1 (N5), 519-537 of SEQ ID NO:1 (N6), 533-551 of SEQ ID NO:1 (N7), 547-565 of SEQ ID NO:1 (N8), 561-579 of SEQ ID NO:1 (N9), 575-593 of SEQ ID NO:1 (N10), 589-607 of SEQ ID NO:1 (N11), 631-649 of SEQ ID NO:1 (N14), 645-663 of SEQ ID NO:1 (N15), 659-677 of SEQ ID NO:1 (N16), 673-691 of SEQ ID NO:1 (N17), 701-719 of SEQ ID NO:1 (N19), 715-733 of SEQ ID NO:1 (N20), 729-747 of SEQ ID NO:1 (N21), 743-761 of SEQ ID NO:1 (N22), 757-775 of SEQ ID NO:1 (N23), 771-789 of SEQ ID NO:1 (N24), 785-803 of SEQ ID NO:1 (N25), 799-817 of SEQ ID NO:1 (N26), 813-831 of SEQ ID NO:1 (N27), 827-845 of SEQ ID NO:1 (N28), 869-887 of SEQ ID NO:1 (C2), 883-901 of SEQ ID NO:1 (C3), 911-929 of SEQ ID NO:1 (C5), 925-943 of SEQ ID NO:1 (C6), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1065-1083 of SEQ ID NO:1 (C16), 1079-1097 of SEQ ID NO:1 (C17), 1107-1125 of SEQ ID NO:1 (C19), 1121-1139 of SEQ ID NO:1 (C20), 1135-1153 of SEQ ID NO:1 (C21), 1149-1167 of SEQ ID NO:1 (C22), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28), 1247-1265 of SEQ ID NO:1 (C29), 1261-1279 of SEQ ID NO:1 (C30), or 1275-1296 of SEQ ID NO:1 (C31). In an aspect of this embodiment, such a BoNT/A peptide is selected from one of the following amino acid sequences: 463-481 of SEQ ID NO:1 (N2), 519-537 of SEQ ID NO:1 (N6), 533-551 of SEQ ID NO:1 (N7), 547-565 of SEQ ID NO:1 (N8), 561-579 of SEQ ID NO:1 (N9), 589-607 of SEQ ID NO:1 (N11), 659-677 of SEQ ID NO:1 (N16), 743-761 of SEQ ID NO:1 (N22), 785-803 of SEQ ID NO:1 (N25), 813-831 of SEQ ID NO:1 (N27), 827-845 of SEQ ID NO:1 (N28), 869-887 of SEQ ID NO:1 (C2), 883-901 of SEQ ID NO:1 (C3), 911-929 of SEQ ID NO:1 (C6), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1121-1139 of SEQ ID NO:1 (C20), 1135-1153 of SEQ ID NO:1 (C21), 1177-1195 of SEQ ID NO:1 (C24), 1261-1279 of SEQ ID NO:1 (C30) or 1275-1296 of SEQ ID NO:1 (C31). In another aspect of this embodiment, such a BoNT/A peptide is selected from one of the following amino acid sequences: 463-481 of SEQ ID NO:1 (N2), 533-551 of SEQ ID NO:1 (N7), 547-565 of SEQ ID NO:1 (N8), 561-579 of SEQ ID NO:1 (N9), 659-677 of SEQ ID NO:1 (N16), 743-761 of SEQ ID NO:1 (N22), 785-803 of SEQ ID NO:1 (N25), 813-831 of SEQ ID NO:1 (N27), 883-901 of SEQ ID NO:1 (C3), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1121-1139 of SEQ ID NO:1 (C20) or 1177-1195 of SEQ ID NO:1 (C24) or 1275-1296 of SEQ ID NO: 1 (C31). In another aspect of this embodiment, such a BoNT/A peptide is selected from one of the following amino acid sequences: 533-551 of SEQ ID NO: 1 (N7), 547-565 of SEQ ID NO: 1 (N8), 743-761 of SEQ ID NO: 1 (N22), 785-803 of SEQ ID NO: 1 (N25), 813-831 of SEQ ID NO: 1 (N27), 995-1013 of SEQ ID NO: 1 (C11); 1051-1069 of SEQ ID NO: 1 (C15), 1177-1195 of SEQ ID NO: 1 (C24), or 1275-1296 of SEQ ID NO: 1 (C31).

In yet another aspect of this embodiment, a tolerogizing composition comprises a tolerizing agent operationally linked to a BoNT/A peptide having a length of at most 60 amino acids and consisting of at least 5 contiguous amino acids selected from one of the following BoNT/A amino acid sequences: 463-481 of SEQ ID NO:1 (N2), 505-523 of SEQ ID NO:1 (N5), 519-537 of SEQ ID NO:1 (N6), 533-551 of SEQ ID NO:1 (N7), 603-621 of SEQ ID NO:1 (N12), 645-663 of SEQ ID NO:1 (N15), 659-677 of SEQ ID NO:1 (N16), 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1079-1097 of SEQ ID NO:1 (C17), 1107-1125 of SEQ ID NO:1 (C19), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28) or 1275-1296 of SEQ ID NO:1 (C31). In yet another aspect of this embodiment, such a BoNT/A peptide is selected from one of the following amino acid sequences: 533-551 of SEQ ID NO:1 (N7), 659-677 of SEQ ID NO:1 (N16), 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1107-1125 of SEQ ID NO:1 (C19), 1163-1181 of SEQ ID NO:1 (C23), 1191-1209 of SEQ ID NO:1 (C25), 1233-1251 of SEQ ID NO:1 (C28) or 1275-1296 of SEQ ID NO:1 (C31). In yet another aspect of this embodiment, such a BoNT/A peptide is selected from one of the following amino acid sequences: 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1079-1097 of SEQ ID NO:1 (C17), 1107-1125 of SEQ ID NO:1 (C19), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28) or 1275-1296 of SEQ ID NO:1 (C31). In yet another aspect of this embodiment, such a BoNT/A peptide is selected from one of the following amino acid sequences: 729-747 of SEQ ID NO:1 (N21) 799-817 of SEQ ID NO:1 (N26), 1065-1083 of SEQ ID NO:1 (C16), 1163-1181 of SEQ ID NO:1 (C23) or 1275-1296 of SEQ ID NO:1 (C31).

In yet another embodiment of the present invention, a tolerogizing composition comprises a tolerizing agent operationally linked to a BoNT/A peptide selected from one of the following BoNT/A amino acid sequences: 449-467 of SEQ ID NO:1 (N1), 463-481 of SEQ ID NO:1 (N2), 491-509 of SEQ ID NO:1 (N4), 505-523 of SEQ ID NO:1 (N5), 519-537 of SEQ ID NO:1 (N6), 533-551 of SEQ ID NO:1 (N7), 547-565 of SEQ ID NO:1 (N8), 561-579 of SEQ ID NO:1 (N9), 575-593 of SEQ ID NO:1 (N10), 589-607 of SEQ ID NO:1 (N11), 631-649 of SEQ ID NO:1 (N14), 645-663 of SEQ ID NO:1 (N15), 659-677 of SEQ ID NO:1 (N16), 673-691 of SEQ ID NO:1 (N17), 701-719 of SEQ ID NO:1 (N19), 715-733 of SEQ ID NO:1 (N20), 729-747 of SEQ ID NO:1 (N21), 743-761 of SEQ ID NO:1 (N22), 757-775 of SEQ ID NO:1 (N23), 771-789 of SEQ ID NO:1 (N24), 785-803 of SEQ ID NO:1 (N25), 799-817 of SEQ ID NO:1 (N26), 813-831 of SEQ ID NO:1 (N27), 827-845 of SEQ ID NO:1 (N28), 869-887 of SEQ ID NO:1 (C2), 883-901 of SEQ ID NO:1 (C3), 911-929 of SEQ ID NO:1 (C5), 925-943 of SEQ ID NO:1 (C6), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1065-1083 of SEQ ID NO:1 (C16), 1079-1097 of SEQ ID NO:1 (C17),1107-1125 of SEQ ID NO:1 (C19), 1121-1139 of SEQ ID NO:1 (C20), 1135-1153 of SEQ ID NO:1 (C21), 1149-1167 of SEQ ID NO:1 (C22), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28),1247-1265 of SEQ ID NO:1 (C29), 1261-1279 of SEQ ID NO:1 (C30), or 1275-1296 of SEQ ID NO:1 (C31). In an aspect of this embodiment, such a BoNT/A peptide is selected from one of the following amino acid sequences: 463-481 of SEQ ID NO:1 (N2), 519-537 of SEQ ID NO:1 (N6), 533-551 of SEQ ID NO:1 (N7), 547-565 of SEQ ID NO:1 (N8), 561-579 of SEQ ID NO:1 (N9), 589-607 of SEQ ID NO:1 (N11), 659-677 of SEQ ID NO:1 (N16), 743-761 of SEQ ID NO:1 (N22), 785-803 of SEQ ID NO:1 (N25), 813-831 of SEQ ID NO:1 (N27), 827-845 of SEQ ID NO:1 (N28), 869-887 of SEQ ID NO:1 (C2), 883-901 of SEQ ID NO:1 (C3), 911-929 of SEQ ID NO:1 (C6), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1121-1139 of SEQ ID NO:1 (C20), 1135-1153 of SEQ ID NO:1 (C21), 1177-1195 of SEQ ID NO:1 (C24), 1261-1279 of SEQ ID NO:1 (C30) or 1275-1296 of SEQ ID NO:1 (C31). In another aspect of this embodiment, such a BoNT/A peptide is selected from one of the following amino acid sequences: 463-481 of SEQ ID NO:1 (N2), 533-551 of SEQ ID NO:1 (N7), 547-565 of SEQ ID NO:1 (N8), 561-579 of SEQ ID NO:1 (N9), 659-677 of SEQ ID NO:1 (N16), 743-761 of SEQ ID NO:1 (N22), 785-803 of SEQ ID NO:1 (N25), 813-831 of SEQ ID NO:1 (N27), 883-901 of SEQ ID NO:1 (C3), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1121-1139 of SEQ ID NO:1 (C20) or 1177-1195 of SEQ ID NO:1 (C24) or 1275-1296 of SEQ ID NO: 1 (C31). In another aspect of this embodiment, such a BoNT/A peptide is selected from one of the following amino acid sequences: 533-551 of SEQ ID NO: 1 (N7), 547-565 of SEQ ID NO: 1 (N8), 743-761 of SEQ ID NO: 1 (N22), 785-803 of SEQ ID NO: 1 (N25), 813-831 of SEQ ID NO: 1 (N27), 995-1013 of SEQ ID NO: 1 (C11); 1051-1069 of SEQ ID NO: 1 (C15), 1177-1195 of SEQ ID NO: 1 (C24), or 1275-1296 of SEQ ID NO: 1 (C31).

In yet another aspect of this embodiment, a tolerogizing composition comprises a tolerizing agent operationally linked to a BoNT/A peptide selected from one of the following BoNT/A amino acid sequences: 463-481 of SEQ ID NO:1 (N2), 505-523 of SEQ ID NO:1 (N5), 519-537 of SEQ ID NO:1 (N6), 533-551 of SEQ ID NO:1 (N7), 603-621 of SEQ ID NO:1 (N12), 645-663 of SEQ ID NO:1 (N15), 659-677 of SEQ ID NO:1 (N16), 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1079-1097 of SEQ ID NO:1 (C17), 1107-1125 of SEQ ID NO:1 (C19), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28) or 1275-1296 of SEQ ID NO:1 (C31). In yet another aspect of this embodiment, such a BoNT/A peptide is selected from one of the following amino acid sequences: 533-551 of SEQ ID NO:1 (N7), 659-677 of SEQ ID NO:1 (N16), 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1107-1125 of SEQ ID NO:1 (C19), 1163-1181 of SEQ ID NO:1 (C23), 1191-1209 of SEQ ID NO:1 (C25), 1233-1251 of SEQ ID NO:1 (C28) or 1275-1296 of SEQ ID NO:1 (C31). In yet another aspect of this embodiment, such a BoNT/A peptide is selected from one of the following amino acid sequences: 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1079-1097 of SEQ ID NO:1 (C17), 1107-1125 of SEQ ID NO:1 (C19), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28) or 1275-1296 of SEQ ID NO:1 (C31). In yet another aspect of this embodiment, such a BoNT/A peptide is selected from one of the following amino acid sequences: 729-747 of SEQ ID NO:1 (N21) 799-817 of SEQ ID NO:1 (N26), 1065-1083 of SEQ ID NO:1 (C16), 1163-1181 of SEQ ID NO:1 (C23) or 1275-1296 of SEQ ID NO:1 (C31).

In yet another embodiment of the present invention, a tolerogizing composition comprises a tolerizing agent operationally linked to a BoNT/A peptide having a length of at most 60 amino acids and consisting of at least 5 contiguous amino acids selected from one of the following BoNT/A amino acid sequences: 449-467 of SEQ ID NO:1 (N1), 463-481 of SEQ ID NO:1 (N2), 491-509 of SEQ ID NO:1 (N4), 505-523 of SEQ ID NO:1 (N5), 519-537 of SEQ ID NO:1 (N6), 533-551 of SEQ ID NO:1 (N7), 547-565 of SEQ ID NO:1 (N8), 561-579 of SEQ ID NO:1 (N9), 575-593 of SEQ ID NO:1 (N10), 589-607 of SEQ ID NO:1 (N11), 631-649 of SEQ ID NO:1 (N14), 645-663 of SEQ ID NO:1 (N15), 659-677 of SEQ ID NO:1 (N16), 673-691 of SEQ ID NO:1 (N17), 701-719 of SEQ ID NO:1 (N19), 715-733 of SEQ ID NO:1 (N20), 729-747 of SEQ ID NO:1 (N21), 743-761 of SEQ ID NO:1 (N22), 757-775 of SEQ ID NO:1 (N23), 771-789 of SEQ ID NO:1 (N24), 785-803 of SEQ ID NO:1 (N25), 799-817 of SEQ ID NO:1 (N26), 813-831 of SEQ ID NO:1 (N27), 827-845 of SEQ ID NO:1 (N28), 869-887 of SEQ ID NO:1 (C2), 883-901 of SEQ ID NO:1 (C3), 911-929 of SEQ ID NO:1 (C5), 925-943 of SEQ ID NO:1 (C6), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1065-1083 of SEQ ID NO:1 (C16), 1079-1097 of SEQ ID NO:1 (C17), 1107-1125 of SEQ ID NO:1 (C19), 1121-1139 of SEQ ID NO:1 (C20), 1135-1153 of SEQ ID NO:1 (C21), 1149-1167 of SEQ ID NO:1 (C22), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28), 1247-1265 of SEQ ID NO:1 (C29), 1261-1279 of SEQ ID NO:1 (C30), or 1275-1296 of SEQ ID NO:1 (C31), or a conservative variant thereof. In an aspect of this embodiment, such a BoNT/A peptide is selected from one of the following amino acid sequences: 463-481 of SEQ ID NO:1 (N2), 519-537 of SEQ ID NO:1 (N6), 533-551 of SEQ ID NO:1 (N7), 547-565 of SEQ ID NO:1 (N8), 561-579 of SEQ ID NO:1 (N9), 589-607 of SEQ ID NO:1 (N11), 659-677 of SEQ ID NO:1 (N16), 743-761 of SEQ ID NO:1 (N22), 785-803 of SEQ ID NO:1 (N25), 813-831 of SEQ ID NO:1 (N27), 827-845 of SEQ ID NO:1 (N28), 869-887 of SEQ ID NO:1 (C2), 883-901 of SEQ ID NO:1 (C3), 911-929 of SEQ ID NO:1 (C6), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1121-1139 of SEQ ID NO:1 (C20), 1135-1153 of SEQ ID NO:1 (C21), 1177-1195 of SEQ ID NO:1 (C24), 1261-1279 of SEQ ID NO:1 (C30) or 1275-1296 of SEQ ID NO:1 (C31), or a conservative variant thereof. In another aspect of this embodiment, such a BoNT/A peptide is selected from one of the following amino acid sequences: 463-481 of SEQ ID NO:1 (N2), 533-551 of SEQ ID NO:1 (N7), 547-565 of SEQ ID NO:1 (N8), 561-579 of SEQ ID NO:1 (N9), 659-677 of SEQ ID NO:1 (N16), 743-761 of SEQ ID NO:1 (N22), 785-803 of SEQ ID NO:1 (N25), 813-831 of SEQ ID NO:1 (N27), 883-901 of SEQ ID NO:1 (C3), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1121-1139 of SEQ ID NO:1 (C20) or 1177-1195 of SEQ ID NO:1 (C24) or 1275-1296 of SEQ ID NO: 1 (C31), or a conservative variant thereof. In another aspect of this embodiment, such a BoNT/A peptide is selected from one of the following amino acid sequences: 533-551 of SEQ ID NO: 1 (N7), 547-565 of SEQ ID NO: 1 (N8), 743-761 of SEQ ID NO: 1 (N22), 785-803 of SEQ ID NO: 1 (N25), 813-831 of SEQ ID NO: 1 (N27), 995-1013 of SEQ ID NO: 1 (C11); 1051-1069 of SEQ ID NO: 1 (C15), 1177-1195 of SEQ ID NO: 1 (C24), or 1275-1296 of SEQ ID NO: 1 (C31), or a conservative variant thereof.

In yet another aspect of this embodiment, a tolerogizing composition comprises a tolerizing agent operationally linked to a BoNT/A peptide having a length of at most 60 amino acids and consisting of at least 5 contiguous amino acids selected from one of the following BoNTIA amino acid sequences: 463-481 of SEQ ID NO:1 (N2), 505-523 of SEQ ID NO:1 (N5), 519-537 of SEQ ID NO:1 (N6), 533-551 of SEQ ID NO:1 (N7), 603-621 of SEQ ID NO:1 (N12), 645-663 of SEQ ID NO:1 (N15), 659-677 of SEQ ID NO:1 (N16), 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1079-1097 of SEQ ID NO:1 (C17), 1107-1125 of SEQ ID NO:1 (C19), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28) or 1275-1296 of SEQ ID NO:1 (C31), or a conservative variant thereof. In yet another aspect of this embodiment, such a BoNT/A peptide is selected from one of the following amino acid sequences: 533-551 of SEQ ID NO:1 (N7), 659-677 of SEQ ID NO:1 (N16), 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1107-1125 of SEQ ID NO:1 (C19), 1163-1181 of SEQ ID NO:1 (C23), 1191-1209 of SEQ ID NO:1 (C25), 1233-1251 of SEQ ID NO:1 (C28) or 1275-1296 of SEQ ID NO:1 (C31), or a conservative variant thereof. In yet another aspect of this embodiment, such a BoNT/A peptide is selected from one of the following amino acid sequences: 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1079-1097 of SEQ ID NO:1 (C17), 1107-1125 of SEQ ID NO:1 (C19), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28) or 1275-1296 of SEQ ID NO:1 (C31), or a conservative variant thereof. In yet another aspect of this embodiment, such a BoNT/A peptide is selected from one of the following amino acid sequences: 729-747 of SEQ ID NO:1 (N21) 799-817 of SEQ ID NO:1 (N26), 1065-1083 of SEQ ID NO:1 (C16), 1163-1181 of SEQ ID NO:1 (C23) or 1275-1296 of SEQ ID NO:1 (C31), or a conservative variant thereof.

In yet another embodiment of the present invention, a tolerogizing composition comprises a tolerizing agent operationally linked to a BoNT/A peptide having a length of at most 60 amino acids and consisting of at least 5 contiguous amino acids selected from one of the following BoNT/A amino acid sequences: 449-467 of SEQ ID NO:1(N1), 463-481 of SEQ ID NO:1 (N2), 491-509 of SEQ ID NO:1 (N4), 505-523 of SEQ ID NO:1 (N5), 519-537 of SEQ ID NO:1 (N6), 533-551 of SEQ ID NO:1 (N7), 547-565 of SEQ ID NO:1 (N8), 561-579 of SEQ ID NO:1 (N9), 575-593 of SEQ ID NO:1 (N10), 589-607 of SEQ ID NO:1 (N11), 631-649 of SEQ ID NO:1 (N14), 645-663 of SEQ ID NO:1 (N15), 659-677 of SEQ ID NO:1 (N16), 673-691 of SEQ ID NO:1 (N17), 701-719 of SEQ ID NO:1 (N19), 715-733 of SEQ ID NO:1 (N20), 729-747 of SEQ ID NO:1 (N21), 743-761 of SEQ ID NO:1 (N22), 757-775 of SEQ ID NO:1 (N23), 771-789 of SEQ ID NO:1 (N24), 785-803 of SEQ ID NO:1 (N25), 799-817 of SEQ ID NO:1 (N26), 813-831 of SEQ ID NO:1 (N27), 827-845 of SEQ ID NO:1 (N28), 869-887 of SEQ ID NO:1 (C2), 883-901 of SEQ ID NO:1 (C3), 911-929 of SEQ ID NO:1 (C5), 925-943 of SEQ ID NO:1 (C6), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1065-1083 of SEQ ID NO:1 (C16), 1079-1097 of SEQ ID NO:1 (C17), 1107-1125 of SEQ ID NO:1 (C19), 1121-1139 of SEQ ID NO:1 (C20), 1135-1153 of SEQ ID NO:1 (C21), 1149-1167 of SEQ ID NO:1 (C22), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28), 1247-1265 of SEQ ID NO:1 (C29), 1261-1279 of SEQ ID NO:1 (C30), or 1275-1296 of SEQ ID NO:1 (C31), or a non-conservative variant thereof. In an aspect of this embodiment, such a BoNT/A peptide is selected from one of the following amino acid sequences: 463-481 of SEQ ID NO:1 (N2), 519-537 of SEQ ID NO:1 (N6), 533-551 of SEQ ID NO:1 (N7), 547-565 of SEQ ID NO:1 (N8), 561-579 of SEQ ID NO:1 (N9), 589-607 of SEQ ID NO:1 (N11), 659-677 of SEQ ID NO:1 (N16), 743-761 of SEQ ID NO:1 (N22), 785-803 of SEQ ID NO:1 (N25), 813-831 of SEQ ID NO:1 (N27), 827-845 of SEQ ID NO:1 (N28), 869-887 of SEQ ID NO:1 (C2), 883-901 of SEQ ID NO:1 (C3), 911-929 of SEQ ID NO:1 (C6), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1121-1139 of SEQ ID NO:1 (C20), 1135-1153 of SEQ ID NO:1 (C21), 1177-1195 of SEQ ID NO:1 (C24), 1261-1279 of SEQ ID NO:1 (C30) or 1275-1296 of SEQ ID NO:1 (C31), or a non-conservative variant thereof. In another aspect of this embodiment, such a BoNT/A peptide is selected from one of the following amino acid sequences: 463-481 of SEQ ID NO:1 (N2), 533-551 of SEQ ID NO:1 (N7), 547-565 of SEQ ID NO:1 (N8), 561-579 of SEQ ID NO:1 (N9), 659-677 of SEQ ID NO:1 (N16), 743-761 of SEQ ID NO:1 (N22), 785-803 of SEQ ID NO:1 (N25), 813-831 of SEQ ID NO:1 (N27), 883-901 of SEQ ID NO:1 (C3), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1121-1139 of SEQ ID NO:1 (C20) or 1177-1195 of SEQ ID NO:1 (C24) or 1275-1296 of SEQ ID NO: 1 (C31), or a non-conservative variant thereof. In another aspect of this embodiment, such a BoNT/A peptide is selected from one of the following amino acid sequences: 533-551 of SEQ ID NO: 1 (N7), 547-565 of SEQ ID NO: 1 (N8), 743-761 of SEQ ID NO: 1 (N22), 785-803 of SEQ ID NO: 1 (N25), 813-831 of SEQ ID NO: 1 (N27), 995-1013 of SEQ ID NO: 1 (C11); 1051-1069 of SEQ ID NO: 1 (C15), 1177-1195 of SEQ ID NO: 1 (C24), or 1275-1296 of SEQ ID NO: 1 (C31), or a non-conservative variant thereof.

In yet another aspect of this embodiment, a tolerogizing composition comprises a tolerizing agent operationally linked to a BoNT/A peptide having a length of at most 60 amino acids and consisting of at least 5 contiguous amino acids selected from one of the following BoNT/A amino acid sequences: 463-481 of SEQ ID NO:1 (N2), 505-523 of SEQ ID NO:1 (N5), 519-537 of SEQ ID NO:1 (N6), 533-551 of SEQ ID NO:1 (N7), 603-621 of SEQ ID NO:1 (N12), 645-663 of SEQ ID NO:1 (N15), 659-677 of SEQ ID NO:1 (N16), 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1079-1097 of SEQ ID NO:1 (C17), 1107-1125 of SEQ ID NO:1 (C19), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28) or 1275-1296 of SEQ ID NO:1 (C31), or a non-conservative variant thereof. In yet another aspect of this embodiment, such a BoNT/A peptide is selected from one of the following amino acid sequences: 533-551 of SEQ ID NO:1 (N7), 659-677 of SEQ ID NO:1 (N16), 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1107-1125 of SEQ ID NO:1 (C19), 1163-1181 of SEQ ID NO:1 (C23), 1191-1209 of SEQ ID NO:1 (C25), 1233-1251 of SEQ ID NO:1 (C28) or 1275-1296 of SEQ ID NO:1 (C31), or a non-conservative variant thereof. In yet another aspect of this embodiment, such a BoNT/A peptide is selected from one of the following amino acid sequences: 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1079-1097 of SEQ ID NO:1 (C17), 1107-1125 of SEQ ID NO:1 (C19), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28) or 1275-1296 of SEQ ID NO:1 (C31), or a non-conservative variant thereof. In yet another aspect of this embodiment, such a BoNT/A peptide is selected from one of the following amino acid sequences: 729-747 of SEQ ID NO:1 (N21) 799-817 of SEQ ID NO:1 (N26), 1065-1083 of SEQ ID NO:1 (C16), 1163-1181 of SEQ ID NO:1 (C23) or 1275-1296 of SEQ ID NO:1 (C31), or a non-conservative variant thereof.

In yet another embodiment of the present invention, a tolerogizing composition comprises a tolerizing agent operationally linked to a BoNT/A peptide having a length of at most 60 amino acids and consisting of at least 5 contiguous amino acids selected from one of the following BoNT/A amino acid sequences: 449-467 of SEQ ID NO:1(N1), 463-481 of SEQ ID NO:1 (N2), 491-509 of SEQ ID NO:1 (N4), 505-523 of SEQ ID NO:1 (N5), 519-537 of SEQ ID NO:1 (N6), 533-551 of SEQ ID NO:1 (N7), 547-565 of SEQ ID NO:1 (N8), 561-579 of SEQ ID NO:1 (N9), 575-593 of SEQ ID NO:1 (N10), 589-607 of SEQ ID NO:1 (N11), 631-649 of SEQ ID NO:1 (N14), 645-663 of SEQ ID NO:1 (N15), 659-677 of SEQ ID NO:1 (N16), 673-691 of SEQ ID NO:1 (N17), 701-719 of SEQ ID NO:1 (N19), 715-733 of SEQ ID NO:1 (N20), 729-747 of SEQ ID NO:1 (N21), 743-761 of SEQ ID NO:1 (N22), 757-775 of SEQ ID NO:1 (N23), 771-789 of SEQ ID NO:1 (N24), 785-803 of SEQ ID NO:1 (N25), 799-817 of SEQ ID NO:1 (N26), 813-831 of SEQ ID NO:1 (N27), 827-845 of SEQ ID NO:1 (N28), 869-887 of SEQ ID NO:1 (C2), 883-901 of SEQ ID NO:1 (C3), 911-929 of SEQ ID NO:1 (C5), 925-943 of SEQ ID NO:1 (C6), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1065-1083 of SEQ ID NO:1 (C16), 1079-1097 of SEQ ID NO:1 (C17), 1107-1125 of SEQ ID NO:1 (C19), 1121-1139 of SEQ ID NO:1 (C20), 1135-1153 of SEQ ID NO:1 (C21), 1149-1167 of SEQ ID NO:1 (C22), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28), 1247-1265 of SEQ ID NO:1 (C29), 1261-1279 of SEQ ID NO:1 (C30), or 1275-1296 of SEQ ID NO:1 (C31), or an immunoreactive fragment thereof, with the proviso that the BoNT/A peptide is not SEQ ID NO:2. In an aspect of this embodiment, such a BoNT/A peptide is selected from one of the following amino acid sequences: 463-481 of SEQ ID NO:1 (N2), 519-537 of SEQ ID NO:1 (N6), 533-551 of SEQ ID NO:1 (N7), 547-565 of SEQ ID NO:1 (N8), 561-579 of SEQ ID NO:1 (N9), 589-607 of SEQ ID NO:1 (N11), 659-677 of SEQ ID NO:1 (N16), 743-761 of SEQ ID NO:1 (N22), 785-803 of SEQ ID NO:1 (N25), 813-831 of SEQ ID NO:1 (N27), 827-845 of SEQ ID NO:1 (N28), 869-887 of SEQ ID NO:1 (C2), 883-901 of SEQ ID NO:1 (C3), 911-929 of SEQ ID NO:1 (C6), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1121-1139 of SEQ ID NO:1 (C20), 1135-1153 of SEQ ID NO:1 (C21), 1177-1195 of SEQ ID NO:1 (C24), 1261-1279 of SEQ ID NO:1 (C30) or 1275-1296 of SEQ ID NO:1 (C31), or an immunoreactive fragment thereof, with the proviso that the BoNT/A peptide is not SEQ ID NO:2. In another aspect of this embodiment, such a BoNT/A peptide is selected from one of the following amino acid sequences: 463-481 of SEQ ID NO:1 (N2), 533-551 of SEQ ID NO:1 (N7), 547-565 of SEQ ID NO:1 (N8), 561-579 of SEQ ID NO:1 (N9), 659-677 of SEQ ID NO:1 (N16), 743-761 of SEQ ID NO:1 (N22), 785-803 of SEQ ID NO:1 (N25), 813-831 of SEQ ID NO:1 (N27), 883-901 of SEQ ID NO:1 (C3), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1121-1139 of SEQ ID NO:1 (C20) or 1177-1195 of SEQ ID NO:1 (C24) or 1275-1296 of SEQ ID NO: 1 (C31), or an immunoreactive fragment thereof, with the proviso that the BoNT/A peptide is not SEQ ID NO:2. In another aspect of this embodiment, such a BoNT/A peptide is selected from one of the following amino acid sequences: 533-551 of SEQ ID NO: 1 (N7), 547-565 of SEQ ID NO: 1 (N8), 743-761 of SEQ ID NO: 1 (N22), 785-803 of SEQ ID NO: 1 (N25), 813-831 of SEQ ID NO: 1 (N27), 995-1013 of SEQ ID NO: 1(C11); 1051-1069 of SEQ ID NO: 1 (C15), 1177-1195 of SEQ ID NO: 1 (C24), or 1275-1296 of SEQ ID NO: 1 (C31), or an immunoreactive fragment thereof, with the proviso that the BoNT/A peptide is not SEQ ID NO:2.

In yet another aspect of this embodiment, a tolerogizing composition comprises a tolerizing agent operationally linked to a BoNT/A peptide having a length of at most 60 amino acids and consisting of at least 5 contiguous amino acids selected from one of the following BoNT/A amino acid sequences: 463-481 of SEQ ID NO:1 (N2), 505-523 of SEQ ID NO:1 (N5), 519-537 of SEQ ID NO:1 (N6), 533-551 of SEQ ID NO:1 (N7), 603-621 of SEQ ID NO:1 (N12), 645-663 of SEQ ID NO:1 (N15), 659-677 of SEQ ID NO:1 (N16), 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1079-1097 of SEQ ID NO:1 (C17), 1107-1125 of SEQ ID NO:1 (C19), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28) or 1275-1296 of SEQ ID NO:1 (C31), or an immunoreactive fragment thereof, with the proviso that the BoNT/A peptide is not SEQ ID NO:2. In yet another aspect of this embodiment, such a BoNT/A peptide is selected from one of the following amino acid sequences: 533-551 of SEQ ID NO:1 (N7), 659-677 of SEQ ID NO:1 (N16), 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1107-1125 of SEQ ID NO:1 (C19), 1163-1181 of SEQ ID NO:1 (C23), 1191-1209 of SEQ ID NO:1 (C25), 1233-1251 of SEQ ID NO:1 (C28) or 1275-1296 of SEQ ID NO:1 (C31), or an immunoreactive fragment thereof, with the proviso that the BoNT/A peptide is not SEQ ID NO:2. In yet another aspect of this embodiment, such a BoNT/A peptide is selected from one of the following amino acid sequences: 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1079-1097 of SEQ ID NO:1 (C17), 1107-1125 of SEQ ID NO:1 (C19), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28) or 1275-1296 of SEQ ID NO:1 (C31), or an immunoreactive fragment thereof, with the proviso that the BoNT/A peptide is not SEQ ID NO:2. In yet another aspect of this embodiment, such a BoNT/A peptide is selected from one of the following amino acid sequences: 729-747 of SEQ ID NO:1 (N21) 799-817 of SEQ ID NO:1 (N26), 1065-1083 of SEQ ID NO:1 (C16), 1163-1181 of SEQ ID NO:1 (C23) or 1275-1296 of SEQ ID NO:1 (C31), or an immunoreactive fragment thereof, with the proviso that the BoNT/A peptide is not SEQ ID NO:2.

In is envisioned that a BoNT/A peptide useful in a tolerogizing composition disclosed in the present specification can have any of a variety of lengths from at least 5 amino acids to at most 60 amino acids. Therefore, aspects of this embodiment may include a BoNT/A peptide with at least, e.g., five amino acids, six amino acids, seven amino acids, eight amino acids, nine amino acids, ten amino acids, 11 amino acids, 12 amino acids, 13 amino acids, 14 amino acids, 15 amino acids, 16 amino acids, 17 amino acids, 18 amino acids, 19 amino acids, 20 amino acids, 25 amino acids, 30 amino acids, 35 amino acids, 40 amino acids, 45 amino acids, 50 amino acids, 55 amino acids or 60 amino acids. Other aspects of this embodiment may include a BoNT/A peptide with at least, e.g., five amino acids of SEQ ID NO:1, six amino acids of SEQ ID NO:1, seven amino acids of SEQ ID NO:1, eight amino acids of SEQ ID NO:1, nine amino acids of SEQ ID NO:1, ten amino acids of SEQ ID NO:1, 11 amino acids of SEQ ID NO:1, 12 amino acids of SEQ ID NO:1, 13 amino acids of SEQ ID NO:1, 14 amino acids of SEQ ID NO:1, 15 amino acids of SEQ ID NO:1, 16 amino acids of SEQ ID NO:1, 17 amino acids of SEQ ID NO:1, 18 amino acids of SEQ ID NO:1, 19 amino acids of SEQ ID NO:1, 20 amino acids of SEQ ID NO:1, 25 amino acids of SEQ ID NO:1, 30 amino acids of SEQ ID NO:1, 35 amino acids of SEQ ID NO:1, 40 amino acids of SEQ ID NO:1, 45 amino acids of SEQ ID NO:1, 50 amino acids of SEQ ID NO:1, 55 amino acids or 60 amino acids of SEQ ID NO:1. In further embodiments, such a BoNT/A peptide of the invention may include a BoNT/A peptide with at least, e.g., five amino acids, six amino acids, seven amino acids, eight amino acids, nine amino acids, ten amino acids, 11 amino acids, 12 amino acids, 13 amino acids, 14 amino acids, 15 amino acids, 16 amino acids, 17 amino acids, 18 amino acids, 19 amino acids, 20 amino acids, 25 amino acids, 30 amino acids, 35 amino acids, 40 amino acids, 45 amino acids, 50 amino acids, 55 amino acids or 60 amino acids and consist of at least 5 contiguous amino acids selected from one of the following BoNT/A amino acid sequences: 449-467 of SEQ ID NO:1(N1), 463-481 of SEQ ID NO:1 (N2), 491-509 of SEQ ID NO:1 (N4), 505-523 of SEQ ID NO:1 (N5), 519-537 of SEQ ID NO:1 (N6), 533-551 of SEQ ID NO:1 (N7), 547-565 of SEQ ID NO:1 (N8), 561-579 of SEQ ID NO:1 (N9), 575-593 of SEQ ID NO:1 (N10), 589-607 of SEQ ID NO:1 (N11), 631-649 of SEQ ID NO:1 (N14), 645-663 of SEQ ID NO:1 (N15), 659-677 of SEQ ID NO:1 (N16), 673-691 of SEQ ID NO:1 (N17), 701-719 of SEQ ID NO:1 (N19), 715-733 of SEQ ID NO:1 (N20), 729-747 of SEQ ID NO:1 (N21), 743-761 of SEQ ID NO:1 (N22), 757-775 of SEQ ID NO:1 (N23), 771-789 of SEQ ID NO:1 (N24), 785-803 of SEQ ID NO:1 (N25), 799-817 of SEQ ID NO:1 (N26), 813-831 of SEQ ID NO:1 (N27), 827-845 of SEQ ID NO:1 (N28), 869-887 of SEQ ID NO:1 (C2), 883-901 of SEQ ID NO:1 (C3), 911-929 of SEQ ID NO:1 (C5), 925-943 of SEQ ID NO:1 (C6), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1065-1083 of SEQ ID NO:1 (C16), 1079-1097 of SEQ ID NO:1 (C17), 1107-1125 of SEQ ID NO:1 (C19), 1121-1139 of SEQ ID NO:1 (C20), 1135-1153 of SEQ ID NO:1 (C21), 1149-1167 of SEQ ID NO:1 (C22), 1163-1181 of SEQ ID NO:1 (C23), 1177-1195 of SEQ ID NO:1 (C24),1191-1209 of SEQ ID NO:1 (C25),1233-1251 of SEQ ID NO:1 (C28), 1247-1265 of SEQ ID NO:1 (C29), 1261-1279 of SEQ ID NO:1 (C30) or 1275-1296 of SEQ ID NO:1 (C31), or a conservative variant, a non-conservative variant, or immunoreactive fragment thereof, with the proviso that the BoNT/A peptide is not SEQ ID NO:2.

In another embodiment of the present invention, a tolerogizing composition can comprise one BoNT/A peptide disclosed in the present specification. In another embodiment of the present invention, a tolerogizing composition can comprise a plurality of BoNT/A peptides disclosed in the present specification. Thus, aspects of this embodiment can include one or more BoNT/A peptides, two or more BoNT/A peptides, three or more BoNT/A peptides, four or more BoNT/A peptides, five or more BoNT/A peptides, six or more BoNT/A peptides, seven or more BoNT/A peptides, eight or more BoNT/A peptides, nine or more BoNT/A peptides, ten or more BoNT/A peptides, 15 or more BoNT/A peptides, 20 or more BoNT/A peptides, 25 or more BoNT/A peptides or 30 or more BoNT/A peptides. In other aspects of this embodiment can include one or more BoNT/A conservative variants, two or more BoNT/A conservative variants, three or more BoNT/A conservative variants, four or more BoNT/A conservative variants, five or more BoNT/A conservative variants, six or more BoNT/A conservative variants, seven or more BoNT/A conservative variants, eight or more BoNT/A conservative variants, nine or more BoNT/A conservative variants, ten or more BoNT/A conservative variants, 15 or more BoNT/A conservative variants, 20 or more BoNT/A conservative variants, 25 or more BoNT/A conservative variants or 30 or more BoNT/A conservative variants. In further aspects of this embodiment can include one or more BoNT/A non-conservative variants, two or more BoNT/A non-conservative variants, three or more BoNT/A non-conservative variants, four or more BoNT/A non-conservative variants, five or more BoNT/A non-conservative variants, six or more BoNT/A non-conservative variants, seven or more BoNT/A non-conservative variants, eight or more BoNT/A non-conservative variants, nine or more BoNT/A non-conservative variants, ten or more BoNT/A non-conservative variants, 15 or more BoNT/A non-conservative variants, 20 or more BoNT/A non-conservative variants, 25 or more BoNT/A non-conservative variants or 30 or more BoNT/A non-conservative variants. In still other aspects of this embodiment can include one or more BoNT/A immunoreactive fragments, two or more BoNT/A immunoreactive fragments, three or more BoNT/A immunoreactive fragments, four or more BoNT/A immunoreactive fragments, five or more BoNT/A immunoreactive fragments, six or more BoNT/A immunoreactive fragments, seven or more BoNT/A immunoreactive fragments, eight or more BoNT/A immunoreactive fragments, nine or more BoNT/A peptides, ten or more BoNT/A immunoreactive fragments, 15 or more BoNT/A immunoreactive fragments, 20 or more BoNT/A immunoreactive fragments, 25 or more BoNT/A immunoreactive fragments or 30 or more BoNT/A immunoreactive fragments. BoNT/A peptides disclosed in the present specification useful for a tolerogizing composition can be selected, for example, depending on immunological factors, such as potency of the peptide in inducing an immune response, and technical factors, such as chemical synthesis yields. It is also understood that the two or more BoNT/A peptides can be provided separately or as part of a compound molecule such as a chimeric peptide or heterologous protein.

In an aspect of this embodiment, a tolerogizing composition comprises a tolerizing agent operationally linked to two or more of the following amino acid sequences: 533-551 of SEQ ID NO:1 (N7), 547-565 of SEQ ID NO:1 (N8), 743-761 of SEQ ID NO:1 (N22), 785-803 of SEQ ID NO:1 (N25), 813-831 of SEQ ID NO:1 (N27), 995-1013 of SEQ ID NO:1 (C11); 1051-1069 of SEQ ID NO:1 (C15), 1177-1195 of SEQ ID NO:1 (C24), and 1275-1296 of SEQ ID NO:1 (C31), or a conservative variant, a non-conservative variant or an immunoreactive fragment thereof, with the proviso that the BoNT/A peptide is not SEQ ID NO:2. In another aspect of this embodiment, one of the selected amino acid sequence is 533-551 of SEQ ID NO:1 (N8) or a conservative variant, a non-conservative variant or an immunoreactive fragment thereof, with the proviso that the BoNT/A peptide is not SEQ ID NO:2. In yet another aspect of this embodiment, the following two amino acid sequences are selected: 533-551 of SEQ ID NO:1 (N8) and 981-999 of SEQ ID NO:1 (C10), or a conservative variant, a non-conservative variant or an immunoreactive fragment thereof, with the proviso that the BoNT/A peptide is not SEQ ID NO:2. In yet another aspect of this embodiment, the following three amino acid sequences are selected: 533-551 of SEQ ID NO:1 (N8), 981-999 of SEQ ID NO:1 (C10) and 1051-1069 of SEQ ID NO:1 (C15), or a conservative variant, a non-conservative variant or an immunoreactive fragment thereof, with the proviso that the BoNT/A peptide is not SEQ ID NO:2. In a further aspect of this embodiment, one of the amino acid sequences selected is 785-803 of SEQ ID NO:1 (N25) or a conservative variant, a non-conservative variant or an immunoreactive fragment thereof, with the proviso that the BoNT/A peptide is not SEQ ID NO:2. In a still further aspect of this embodiment, the following two amino acid sequences are selected: 785-803 of SEQ ID NO:1 (N25) and 981-999 of SEQ ID NO:1 (Co), or a conservative variant, a non-conservative variant or an immunoreactive fragment thereof, with the proviso that the BoNT/A peptide is not SEQ ID NO:2. In a still further aspect of this embodiment, the following three amino acid sequences are selected: 785-803 of SEQ ID NO:1 (N25), 981-999 of SEQ ID NO:1 (C10) and 1051-1069 of SEQ ID NO:1 (C15), or a conservative variant, a non-conservative variant or an immunoreactive fragment thereof, with the proviso that the BoNT/A peptide is not SEQ ID NO:2. In an additional aspect of this embodiment, one of the amino acid sequences selected is 813-831 of SEQ ID NO:1 (N27) or a conservative variant, a non-conservative variant or an immunoreactive fragment thereof, with the proviso that the BoNT/A peptide is not SEQ ID NO:2. In another additional aspect of this embodiment, the following two amino acid sequences are selected: 813-831 of SEQ ID NO:1 (N27) and 981-999 of SEQ ID NO:1 (C10), or a conservative variant, a non-conservative variant or an immunoreactive fragment thereof, with the proviso that the BoNT/A peptide is not SEQ ID NO:2. In another additional aspect of this embodiment, the following three amino acid sequences are selected: 813-831 of SEQ ID NO:1 (N27), 981-999 of SEQ ID NO:1 (C10) and 1051-1069 of SEQ ID NO:1 (C15), or a conservative variant, a non-conservative variant or an immunoreactive fragment thereof, with the proviso that the BoNT/A peptide is not SEQ ID NO:2.

In an aspect of this embodiment, a tolerogizing composition comprises a tolerizing agent operationally linked to two or more of the following amino acid sequences: 659-677 of SEQ ID NO:1 (N16), 729-747 of SEQ ID NO:1 (N21), 799-817 of SEQ ID NO:1 (N26), 1065-1083 of SEQ ID NO:1 (C16), 1163-1181 of SEQ ID NO:1 (C23), and 1275-1296 of SEQ ID NO:1 (C31), or a conservative variant, a non-conservative variant or an immunoreactive fragment thereof, with the proviso that the BoNT/A peptide is not SEQ ID NO:2. In another aspect of this embodiment, one of the amino acid sequences selected is 1065-1083 of SEQ ID NO:1 (C16) or a conservative variant, a non-conservative variant or an immunoreactive fragment thereof, with the proviso that the BoNT/A peptide is not SEQ ID NO:2. In yet another aspect of this embodiment, the following two amino acid sequences are selected: 1065-1083 of SEQ ID NO:1 (C16) and 1163-1181 of SEQ ID NO:1 (C23), or a conservative variant, a non-conservative variant or an immunoreactive fragment thereof, with the proviso that the BoNT/A peptide is not SEQ ID NO:2. In yet another aspect of this embodiment, the following three amino acid sequences are selected: 1065-1083 of SEQ ID NO:1 (C16), 1163-1181 of SEQ ID NO:1 (C23) and 1275-1296 of SEQ ID NO:1 (C31), or a conservative variant, a non-conservative variant or an immunoreactive fragment thereof, with the proviso that the BoNT/A peptide is not SEQ ID NO:2. In a further aspect of this embodiment, one of the amino acid sequences selected is 799-817 of SEQ ID NO:1 (N26) or a conservative variant, a non-conservative variant or an immunoreactive fragment thereof, with the proviso that the BoNT/A peptide is not SEQ ID NO:2. In a still further aspect of this embodiment, the following two amino acid sequences are selected: 799-817 of SEQ ID NO:1 (N26) and 1065-1083 of SEQ ID NO:1 (C16), or a conservative variant, a non-conservative variant or an immunoreactive fragment thereof, with the proviso that the BoNT/A peptide is not SEQ ID NO:2. In a still further aspect of this embodiment, the following three amino acid sequences are selected: 799-817 of SEQ ID NO:1 (N26), 1065-1083 of SEQ ID NO:1 (C16) and 1163-1181 of SEQ ID NO:1 (C23), or a conservative variant, a non-conservative variant or an immunoreactive fragment thereof, with the proviso that the BoNT/A peptide is not SEQ ID NO:2. In an additional aspect of this embodiment, one of the amino acid sequences selected is 729-747 of SEQ ID NO:1 (N21) or a conservative variant, a non-conservative variant or an immunoreactive fragment thereof, with the proviso that the BoNT/A peptide is not SEQ ID NO:2. In another additional aspect of this embodiment, the following two amino acid sequences are selected: 729-747 of SEQ ID NO:1 (N21) and 1065-1083 of SEQ ID NO:1 (C16), or a conservative variant, a non-conservative variant or an immunoreactive fragment thereof, with the proviso that the BoNT/A peptide is not SEQ ID NO:2. In another additional aspect of this embodiment, the following three amino acid sequences are selected: 729-747 of SEQ ID NO:1 (N21), 1065-1083 of SEQ ID NO:1 (C16) and 1163-1181 of SEQ ID NO:1 (C23), or a conservative variant, a non-conservative variant or an immunoreactive fragment thereof, with the proviso that the BoNT/A peptide is not SEQ ID NO:2.

It is also envisioned that any and all combinations of BoNT/A peptides disclosed in the specification, including, e.g., BoNT/A peptides of SEQ ID NO:1, BoNT/A conservative variants, BoNT/A non-conservative variants and BoNT/A immunoreactive fragments, can be used in a tolerogizing composition. Thus, aspects of this embodiment include one or more BoNT/A peptides comprising one or more BoNT/A peptides of SEQ ID NO: 1 and one or more BoNT/A conservative variants; one or more BoNT/A peptides of SEQ ID NO: 1 and one or more BoNT/A non-conservative variants; one or more BoNT/A peptides of SEQ ID NO: 1 and one or more BoNT/A immunoreactive fragments; one or more BoNT/A conservative variants and one or more BoNT/A non-conservative variants; one or more BoNT/A conservative variants and one or more BoNT/A immunoreactive fragments; one or more BoNT/A non-conservative variants and one or more BoNT/A immunoreactive fragments; one or more BoNT/A peptides of SEQ ID NO: 1, one or more BoNT/A conservative variants and one or more BoNT/A non-conservative variants; one or more BoNT/A peptides of SEQ ID NO: 1, one or more BoNT/A conservative variants and one or more BoNT/A immunoreactive fragments; one or more BoNT/A peptides of SEQ ID NO: 1, one or more BoNT/A non-conservative variants and one or more BoNT/A immunoreactive fragments; one or more BoNT/A conservative variants, one or more BoNT/A non-conservative variants and one or more BoNT/A immunoreactive fragments; or one or more BoNT/A peptides of SEQ ID NO: 1, one or more BoNT/A conservative variants, one or more BoNT/A non-conservative variants and one or more BoNT/A immunoreactive fragments.

It is envisioned that a wide variety of tolerogizing agents can be useful in a tolerogizing composition disclosed in the present specification. As used herein, the term “tolerogizing agent” means a molecule, compound or polymer that causes, promotes or enhances tolerogenic activity when combined with a BoNT/A peptide disclosed in the present specification. As non-limiting examples, a tolerogizing agent can be a liquid, solid, or emulsion, depending, for example, on the route of administration and physical properties of the tolerogizing agent. A tolerogizing agent is operationally linked to a BoNT/A peptide disclosed in the present specification. As used herein, the term “operationally linked” means to covalently attach a tolerogizing agent to a BoNT/A peptide in a manner that renders the peptide tolerogenic. Such tolerogizing agents can be operationally linked to a BoNT/A peptide, for example, as described in M. Zouhair Atassi & Tetsuo Ashizawa, PVA or PEG Conjugates of Peptides for Epitope-Specific Immunosuppression, U.S. Pat. No. 6,048,529 (Apr. 11, 2000); Emilio Barbera-Guillem & M. Bud Nelson, Compositions and Methods for Tolerization in Immune Complex-Mediated Disease Progression, U.S. Pat. No. 6,245,752 (Jun. 12, 2001); and Edward Jess Victoria et al., APL Immunoreactive Peptides, Conjugates Thereof and Methods of Treatment for APL Antibody-Mediated Pathologies, U.S. Pat. No. 6,410,775 (Jun. 25, 2002), which are hereby incorporated by reference in their entirety. A variety of tolerogizing agents are useful in the invention including, without limitation, polyethylene glycol (PEG), monomethoxypolyethylene glycol (mPEG), and polyvinyl alcohol (PVA). Additional molecules are also known in the art to cause, promote or enhance tolerance, see, e.g., Paul A. Barstad, & Gilbert M. Iverson, Composition For Inducing Humoral Anergy to an Immunogen Comprising a T Cell Epitope-Deficient Analog of the Immunogen Conjugated to a Nonimmunogenic Carrier, U.S. Pat. No. 5,268,454 (Dec. 7, 1993); M. Zouhair Atassi & Tetsuo Ashizawa, PVA or PEG Conjugates of Peptides for Epitope-Specific Immunosuppression, U.S. Pat. No. 6,048,529 (Apr. 11, 2000); and Stephen M. Coutts et al., Composition for Inducing Humoral Anergy to an Immunogen Comprising a T Cell Epitope-Deficient Analog of the Immunogen Conjugated to a Nonimmunogenic Valency Platform Molecule, U.S. Pat. No.6,060,056 (May 9, 2000), which are hereby incorporated by reference in their entirety.

BoNT/A peptides disclosed in the present specification included in a tolerogizing composition can be selected, for example, depending on immunological factors, such as potency of the peptide in inducing a tolerogizing response, and technical factors, such as chemical synthesis yields. As used herein in reference to BoNT/A, the term “tolerogizing response” means a BoNT/A peptide of SEQ ID NO:1, a BoNT/A conservative variant, a BoNT/A non-conservative variant or a BoNT/A immunoreactive fragment that has tolerogenic activity as defined by the ability either alone, or in combination with one or more other molecules, to produce a decreased immunological response. A BoNT/A peptide exhibiting a tolerogizing response can be identified using any of a variety of assays, including in vitro assays such as T-cell proliferation or cytokine secretion assays and in vivo assays such as the induction of tolerance in animal models of botulinum toxicity. T-cell proliferation assays, for example, are well recognized in the art as predictive of tolerogenic activity (see, for example, H. Miyahara et al., Identification and Characterization Of A Major Tolerogenic T-Cell Epitope of Type II Collagen That Suppresses Arthritis in B10.RIII Mice, 86(1) IMMUNOLOGY 110-115 (1995); and Knut E. A. Lundin et al, Gliadin-Specific, HLA-DQ(Alpha 1*0501,Beta 1*0201) Restricted T Cells Isolated From the Small Intestinal Mucosa of Celiac Disease Patients, 178(1) J. EXP. MED. 187-196 (1993), which are hereby incorporated by reference in their entirety. A T-cell proliferation assay can be performed, for example, by culturing T-cells with irradiated antigen-presenting cells, such as normal spleen cells, in microtiter wells for 3 days with varying concentrations of the BoNT/A fragment to be assayed; adding ³H-thymidine; and measuring incorporation of ³H-thymidine into DNA.

A BoNT/A peptide exhibiting a tolerogizing response can also be identified using a T-cell cytokine secretion assay known in the art. In such an assay, T cells can be cultured, for example, with irradiated antigen-presenting cells in microtiter wells with varying concentrations of the fragment of interest and, after three days, the culture supernatants can be assayed for IL-2, IL-4 or IFN-γ as described in C. Czerkinsky et al., Detection of Human Cytokine-Secreting Cells in Distinct Anatomical Compartments, 119 IMMUNOL. REV. 5-22 (1991).

A BoNT/A peptide exhibiting a tolerogizing response can additionally be identified by its ability to induce tolerance in vivo, as indicated by a decreased immunological response, which can be a decreased T-cell response, such as a decreased proliferative response or cytokine secretion response as described above, or a decreased antibody titer to the antigen. A neonatal or adult mouse can be tolerized with a fragment of a BoNT/A peptide, and a T-cell response or anti-BoNT/A antibody titer can be assayed after challenging by immunization. As an example, a neonatal mouse can be tolerized within 48 hours of birth by intraperitoneal administration of about 100 pg of a fragment of a BoNT/A peptide emulsified with incomplete Freund's adjuvant and subsequently immunized with BoNT/A toxin at about 8 weeks of age, see, for example, Miyahara et al., supra, 1995. An adult mouse can be tolerized intravenously with about 0.33 mg of a fragment of a BoNT/A peptide, administered daily for three days (total dose 1 mg), and immunized one week later with BoNT/A. A decreased T-cell response, such as decreased proliferation or cytokine secretion, which indicates tolerogenic activity, can be measured using T-cells harvested 10 days after immunization. In addition, a decreased anti-BoNT/A antibody titer, which also indicates tolerogenic activity, can be assayed using blood harvested 4-8 weeks after immunization. Methods for assaying a T-cell response or anti-BoNT/A antibody titer are described above and are well known in the art.

Several well-accepted models of botulinum toxicity can be useful in identifying a BoNT/A peptide exhibiting a tolerogizing response. Such models include, without limitation, rodent, rabbit and monkey models of foodborne botulism, rodent and chicken models of infant botulism and rodent models of wound botulism, which are described, for example, in Simpson (Ed.) Botulinum Neurotoxin and Tetanus Toxin Academic Press, Inc., San Diego, Calif. (1989). The skilled person understands that these and a variety of other well known in vitro and in vivo assays can be useful for identifying a tolerogenic fragment of a BoNT/A peptide.

IV. BoNT/A Vaccine Compositions

The present invention further provides vaccine compositions useful, for example, for inducing specific immunity against one or more botulinum toxins such as BoNT/A. Such specific immunity can protect a human or other mammal from intoxication produced by exposure to botulinum toxin. As used herein, the term “vaccine” means a composition which, when administered to a human or other mammal, stimulates an immune response against an antigen. The term “immune response” refers to any response to a vaccine composition or other immunogenic compound by the immune system of a vertebrate subject. Exemplary immune responses include, but not limited to cellular as well as local and systemic humoral immunity, such as CTL responses, including antigen-specific induction of CD8+ CTLs, helper T-cell responses, including T-cell proliferative responses and cytokine release, and B-cell responses including antibody response. The term “inducing an immune response” refers to administration of a vaccine composition or other immunogenic compound or a nucleic acid encoding the vaccine composition or other immunogenic compound, wherein an immune response is affected, i.e., stimulated, initiated or induced. A vaccine composition can be useful, for example, for preventing or ameliorating intoxication produced by unwanted exposure to botulinum toxin. Vaccination using peptides has been shown to effectively block the effect of protein toxins. See, for example, Behzod Z. Dolimbek & M. Zouhair Atassi, 13(5) J. PROT. CHEM. 490-493 (1994); M. Zouhair Atassi et al., Antibody and T-Cell Recognition of Alpha-Bungarotoxin and its Synthetic Loop-Peptides, 32(12) MOL. IMMUNOL. 919-929 (1995); and Behzod Z. Dolimbek et al., Protection Against Alpha-Bungarotoxin Poisoning by Immunization with Synthetic Toxin Peptides, 33(7-8) MOL. IMMUNOL. 681-689 (1996).

Thus, the present invention further provides a vaccine composition comprising a BoNT/A peptide disclosed in the present specification. In one embodiment of the present invention, a vaccine composition comprises a BoNT/A peptide having a length of at most 60 amino acids and consisting of at least 5 contiguous amino acids selected from one of the following BoNT/A amino acid sequences: 449-467 of SEQ ID NO:1(N1), 463-481 of SEQ ID NO:1 (N2), 491-509 of SEQ ID NO:1 (N4), 505-523 of SEQ ID NO:1 (N5), 519-537 of SEQ ID NO:1 (N6), 533-551 of SEQ ID NO:1 (N7), 547-565 of SEQ ID NO:1 (N8), 561-579 of SEQ ID NO:1 (N9), 575-593 of SEQ ID NO:1 (N10), 589-607 of SEQ ID NO:1 (N11), 631-649 of SEQ ID NO:1 (N14), 645-663 of SEQ ID NO:1 (N15), 659-677 of SEQ ID NO:1 (N16), 673-691 of SEQ ID NO:1 (N17), 701-719 of SEQ ID NO:1 (N19), 715-733 of SEQ ID NO:1 (N20), 729-747 of SEQ ID NO:1 (N21), 743-761 of SEQ ID NO:1 (N22), 757-775 of SEQ ID NO:1 (N23), 771-789 of SEQ ID NO:1 (N24), 785-803 of SEQ ID NO:1 (N25), 799-817 of SEQ ID NO:1 (N26), 813-831 of SEQ ID NO:1 (N27), 827-845 of SEQ ID NO:1 (N28), 869-887 of SEQ ID NO:1 (C2), 883-901 of SEQ ID NO:1 (C3), 911-929 of SEQ ID NO:1 (C5), 925-943 of SEQ ID NO:1 (C6), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1065-1083 of SEQ ID NO:1 (C16), 1079-1097 of SEQ ID NO:1 (C17), 1107-1125 of SEQ ID NO:1 (C19), 1121-1139 of SEQ ID NO:1 (C20), 1135-1153 of SEQ ID NO:1 (C21), 1149-1167 of SEQ ID NO:1 (C22), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28),1247-1265 of SEQ ID NO:1 (C29),1261-1279 of SEQ ID NO:1 (C30), or 1275-1296 of SEQ ID NO:1 (C31), or a conservative variant or immunoreactive fragment thereof, with the proviso that the BoNT/A peptide is not SEQ ID NO:2.

In another embodiment of the present invention, a vaccine composition comprises a BoNT/A peptide having a length of at most 60 amino acids and consisting of at least 5 contiguous amino acids selected from one of the following BoNT/A amino acid sequences: 449-467 of SEQ ID NO:1(N1), 463-481 of SEQ ID NO:1 (N2), 491-509 of SEQ ID NO:1 (N4), 505-523 of SEQ ID NO:1 (N5), 519-537 of SEQ ID NO:1 (N6), 533-551 of SEQ ID NO:1 (N7), 547-565 of SEQ ID NO:1 (N8), 561-579 of SEQ ID NO:1 (N9), 575-593 of SEQ ID NO:1 (N10), 589-607 of SEQ ID NO:1 (N11), 631-649 of SEQ ID NO:1 (N14), 645-663 of SEQ ID NO:1 (N15), 659-677 of SEQ ID NO:1 (N16), 673-691 of SEQ ID NO:1 (N17), 701-719 of SEQ ID NO:1 (N19), 715-733 of SEQ ID NO:1 (N20), 729-747 of SEQ ID NO:1 (N21), 743-761 of SEQ ID NO:1 (N22), 757-775 of SEQ ID NO:1 (N23), 771-789 of SEQ ID NO:1 (N24), 785-803 of SEQ ID NO:1 (N25), 799-817 of SEQ ID NO:1 (N26), 813-831 of SEQ ID NO:1 (N27), 827-845 of SEQ ID NO:1 (N28), 869-887 of SEQ ID NO:1 (C2), 883-901 of SEQ ID NO:1 (C3), 911-929 of SEQ ID NO:1 (C5), 925-943 of SEQ ID NO:1 (C6), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1065-1083 of SEQ ID NO:1 (C16), 1079-1097 of SEQ ID NO:1 (C17),1107-1125 of SEQ ID NO:1 (C19),1121-1139 of SEQ ID NO:1 (C20),1135-1153 of SEQ ID NO:1 (C21), 1149-1167 of SEQ ID NO:1 (C22), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28), 1247-1265 of SEQ ID NO:1 (C29), 1261-1279 of SEQ ID NO:1 (C30), or 1275-1296 of SEQ ID NO:1 (C31). In an aspect of this embodiment, such a BoNT/A peptide is selected from one of the following amino acid sequences: 463-481 of SEQ ID NO:1 (N2), 519-537 of SEQ ID NO:1 (N6), 533-551 of SEQ ID NO:1 (N7), 547-565 of SEQ ID NO:1 (N8), 561-579 of SEQ ID NO:1 (N9), 589-607 of SEQ ID NO:1 (N11), 659-677 of SEQ ID NO:1 (N16), 743-761 of SEQ ID NO:1 (N22), 785-803 of SEQ ID NO:1 (N25), 813-831 of SEQ ID NO:1 (N27), 827-845 of SEQ ID NO:1 (N28), 869-887 of SEQ ID NO:1 (C2), 883-901 of SEQ ID NO:1 (C3), 911-929 of SEQ ID NO:1 (C6), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1121-1139 of SEQ ID NO:1 (C20), 1135-1153 of SEQ ID NO:1 (C21), 1177-1195 of SEQ ID NO:1 (C24), 1261-1279 of SEQ ID NO:1 (C30) or 1275-1296 of SEQ ID NO:1 (C31). In another aspect of this embodiment, such a BoNT/A peptide is selected from one of the following amino acid sequences: 463-481 of SEQ ID NO:1 (N2), 533-551 of SEQ ID NO:1 (N7), 547-565 of SEQ ID NO:1 (N8), 561-579 of SEQ ID NO:1 (N9), 659-677 of SEQ ID NO:1 (N16), 743-761 of SEQ ID NO:1 (N22), 785-803 of SEQ ID NO:1 (N25), 813-831 of SEQ ID NO:1 (N27), 883-901 of SEQ ID NO:1 (C3), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1121-1139 of SEQ ID NO:1 (C20) or 1177-1195 of SEQ ID NO:1 (C24) or 1275-1296 of SEQ ID NO: 1 (C31). In another aspect of this embodiment, such a BoNT/A peptide is selected from one of the following amino acid sequences: 533-551 of SEQ ID NO: 1 (N7), 547-565 of SEQ ID NO: 1 (N8), 743-761 of SEQ ID NO: 1 (N22), 785-803 of SEQ ID NO: 1 (N25), 813-831 of SEQ ID NO: 1 (N27), 995-1013 of SEQ ID NO: 1 (C11); 1051-1069 of SEQ ID NO: 1 (C15), 1177-1195 of SEQ ID NO: 1 (C24), or 1275-1296 of SEQ ID NO: 1 (C31).

In yet another aspect of this embodiment, a vaccine composition comprises a BoNT/A peptide having a length of at most 60 amino acids and consisting of at least 5 contiguous amino acids selected from one of the following BoNT/A amino acid sequences: 463-481 of SEQ ID NO:1 (N2), 505-523 of SEQ ID NO:1 (N5), 519-537 of SEQ ID NO:1 (N6), 533-551 of SEQ ID NO:1 (N7), 603-621 of SEQ ID NO:1 (N12), 645-663 of SEQ ID NO:1 (N15), 659-677 of SEQ ID NO:1 (N16), 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1079-1097 of SEQ ID NO:1 (C17), 1107-1125 of SEQ ID NO:1 (C19), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28) or 1275-1296 of SEQ ID NO:1 (C31). In yet another aspect of this embodiment, such a BoNT/A peptide is selected from one of the following amino acid sequences: 533-551 of SEQ ID NO:1 (N7), 659-677 of SEQ ID NO:1 (N16), 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1107-1125 of SEQ ID NO:1 (C19), 1163-1181 of SEQ ID NO:1 (C23), 1191-1209 of SEQ ID NO:1 (C25), 1233-1251 of SEQ ID NO:1 (C28) or 1275-1296 of SEQ ID NO:1 (C31). In yet another aspect of this embodiment, such a BoNT/A peptide is selected from one of the following amino acid sequences: 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1079-1097 of SEQ ID NO:1 (C17), 1107-1125 of SEQ ID NO:1 (C19), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28) or 1275-1296 of SEQ ID NO:1 (C31). In yet another aspect of this embodiment, such a BoNT/A peptide is selected from one of the following amino acid sequences: 729-747 of SEQ ID NO:1 (N21) 799-817 of SEQ ID NO:1 (N26), 1065-1083 of SEQ ID NO:1 (C16), 1163-1181 of SEQ ID NO:1 (C23) or 1275-1296 of SEQ ID NO:1 (C31).

In yet another embodiment of the present invention, a vaccine composition comprises a BoNT/A peptide selected from one of the following BoNT/A amino acid sequences: 449-467 of SEQ ID NO:1(N1), 463-481 of SEQ ID NO:1 (N2), 491-509 of SEQ ID NO:1 (N4), 505-523 of SEQ ID NO:1 (N5), 519-537 of SEQ ID NO:1 (N6), 533-551 of SEQ ID NO:1 (N7), 547-565 of SEQ ID NO:1 (N8), 561-579 of SEQ ID NO:1 (N9), 575-593 of SEQ ID NO:1 (N10), 589-607 of SEQ ID NO:1 (N11), 631-649 of SEQ ID NO:1 (N14), 645-663 of SEQ ID NO:1 (N15), 659-677 of SEQ ID NO:1 (N16), 673-691 of SEQ ID NO:1 (N17), 701-719 of SEQ ID NO:1 (N19), 715-733 of SEQ ID NO:1 (N20), 729-747 of SEQ ID NO:1 (N21), 743-761 of SEQ ID NO:1 (N22), 757-775 of SEQ ID NO:1 (N23), 771-789 of SEQ ID NO:1 (N24), 785-803 of SEQ ID NO:1 (N25), 799-817 of SEQ ID NO:1 (N26), 813-831 of SEQ ID NO:1 (N27), 827-845 of SEQ ID NO:1 (N28), 869-887 of SEQ ID NO:1 (C2), 883-901 of SEQ ID NO:1 (C3), 911-929 of SEQ ID NO:1 (C5), 925-943 of SEQ ID NO:1 (C6), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1065-1083 of SEQ ID NO:1 (C16), 1079-1097 of SEQ ID NO:1 (C17), 1107-1125 of SEQ ID NO:1 (C19), 1121-1139 of SEQ ID NO:1 (C20), 1135-1153 of SEQ ID NO:1 (C21), 1149-1167 of SEQ ID NO:1 (C22), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28), 1247-1265 of SEQ ID NO:1 (C29), 1261-1279 of SEQ ID NO:1 (C30), or 1275-1296 of SEQ ID NO:1 (C31). In an aspect of this embodiment, such a BoNT/A peptide is selected from one of the following amino acid sequences: 463-481 of SEQ ID NO:1 (N2), 519-537 of SEQ ID NO:1 (N6), 533-551 of SEQ ID NO:1 (N7), 547-565 of SEQ ID NO:1 (N8), 561-579 of SEQ ID NO:1 (N9), 589-607 of SEQ ID NO:1 (N11), 659-677 of SEQ ID NO:1 (N16), 743-761 of SEQ ID NO:1 (N22), 785-803 of SEQ ID NO:1 (N25), 813-831 of SEQ ID NO:1 (N27), 827-845 of SEQ ID NO:1 (N28), 869-887 of SEQ ID NO:1 (C2), 883-901 of SEQ ID NO:1 (C3), 911-929 of SEQ ID NO:1 (C6), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1121-1139 of SEQ ID NO:1 (C20), 1135-1153 of SEQ ID NO:1 (C21), 1177-1195 of SEQ ID NO:1 (C24), 1261-1279 of SEQ ID NO:1 (C30) or 1275-1296 of SEQ ID NO:1 (C31). In another aspect of this embodiment, such a BoNT/A peptide is selected from one of the following amino acid sequences: 463-481 of SEQ ID NO:1 (N2), 533-551 of SEQ ID NO:1 (N7), 547-565 of SEQ ID NO:1 (N8), 561-579 of SEQ ID NO:1 (N9), 659-677 of SEQ ID NO:1 (N16), 743-761 of SEQ ID NO:1 (N22), 785-803 of SEQ ID NO:1 (N25), 813-831 of SEQ ID NO:1 (N27), 883-901 of SEQ ID NO:1 (C3), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1121-1139 of SEQ ID NO:1 (C20) or 1177-1195 of SEQ ID NO:1 (C24) or 1275-1296 of SEQ ID NO: 1 (C31). In another aspect of this embodiment, such a BoNT/A peptide is selected from one of the following amino acid sequences: 533-551 of SEQ ID NO: 1 (N7), 547-565 of SEQ ID NO: 1 (N8), 743-761 of SEQ ID NO: 1 (N22), 785-803 of SEQ ID NO: 1 (N25), 813-831 of SEQ ID NO: 1 (N27), 995-1013 of SEQ ID NO: 1 (C11); 1051-1069 of SEQ ID NO: 1 (C15), 1177-1195 of SEQ ID NO: 1 (C24), or 1275-1296 of SEQ ID NO: 1 (C31).

In yet another aspect of this embodiment, a vaccine composition comprises a BoNT/A peptide selected from one of the following BoNT/A amino acid sequences: 463-481 of SEQ ID NO:1 (N2), 505-523 of SEQ ID NO:1 (N5), 519-537 of SEQ ID NO:1 (N6), 533-551 of SEQ ID NO:1 (N7), 603-621 of SEQ ID NO:1 (N12), 645-663 of SEQ ID NO:1 (N15), 659-677 of SEQ ID NO:1 (N16), 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1079-1097 of SEQ ID NO:1 (C17), 1107-1125 of SEQ ID NO:1 (C19), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28) or 1275-1296 of SEQ ID NO:1 (C31). In yet another aspect of this embodiment, such a BoNT/A peptide is selected from one of the following amino acid sequences: 533-551 of SEQ ID NO:1 (N7), 659-677 of SEQ ID NO:1 (N16), 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1107-1125 of SEQ ID NO:1 (C19), 1163-1181 of SEQ ID NO:1 (C23), 1191-1209 of SEQ ID NO:1 (C25), 1233-1251 of SEQ ID NO:1 (C28) or 1275-1296 of SEQ ID NO:1 (C31). In yet another aspect of this embodiment, such a BoNT/A peptide is selected from one of the following amino acid sequences: 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1079-1097 of SEQ ID NO:1 (C17), 1107-1125 of SEQ ID NO:1 (C19), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28) or 1275-1296 of SEQ ID NO:1 (C31). In yet another aspect of this embodiment, such a BoNT/A peptide is selected from one of the following amino acid sequences: 729-747 of SEQ ID NO:1 (N21) 799-817 of SEQ ID NO:1 (N26), 1065-1083 of SEQ ID NO:1 (C16), 1163-1181 of SEQ ID NO:1 (C23) or 1275-1296 of SEQ ID NO:1 (C31).

In yet another embodiment of the present invention, a vaccine composition comprises a BoNT/A peptide having a length of at most 60 amino acids and consisting of at least 5 contiguous amino acids selected from one of the following BoNT/A amino acid sequences: 449-467 of SEQ ID NO:1(N1), 463-481 of SEQ ID NO:1 (N2), 491-509 of SEQ ID NO:1 (N4), 505-523 of SEQ ID NO:1 (N5), 519-537 of SEQ ID NO:1 (N6), 533-551 of SEQ ID NO:1 (N7), 547-565 of SEQ ID NO:1 (N8), 561-579 of SEQ ID NO:1 (N9), 575-593 of SEQ ID NO:1 (N10), 589-607 of SEQ ID NO:1 (N11), 631-649 of SEQ ID NO:1 (N14), 645-663 of SEQ ID NO:1 (N15), 659-677 of SEQ ID NO:1 (N16), 673-691 of SEQ ID NO:1 (N17), 701-719 of SEQ ID NO:1 (N19), 715-733 of SEQ ID NO:1 (N20), 729-747 of SEQ ID NO:1 (N21), 743-761 of SEQ ID NO:1 (N22), 757-775 of SEQ ID NO:1 (N23), 771-789 of SEQ ID NO:1 (N24), 785-803 of SEQ ID NO:1 (N25), 799-817 of SEQ ID NO:1 (N26), 813-831 of SEQ ID NO:1 (N27), 827-845 of SEQ ID NO:1 (N28), 869-887 of SEQ ID NO:1 (C2), 883-901 of SEQ ID NO:1 (C3), 911-929 of SEQ ID NO:1 (C5), 925-943 of SEQ ID NO:1 (C6), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1065-1083 of SEQ ID NO:1 (C16), 1079-1097 of SEQ ID NO:1 (C17), 1107-1125 of SEQ ID NO:1 (C19), 1121-1139 of SEQ ID NO:1 (C20), 1135-1153 of SEQ ID NO:1 (C21), 1149-1167 of SEQ ID NO:1 (C22), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28), 1247-1265 of SEQ ID NO:1 (C29), 1261-1279 of SEQ ID NO:1 (C30), or 1275-1296 of SEQ ID NO:1 (C31), or a conservative variant thereof. In an aspect of this embodiment, such a BoNT/A peptide is selected from one of the following amino acid sequences: 463-481 of SEQ ID NO:1 (N2), 519-537 of SEQ ID NO:1 (N6), 533-551 of SEQ ID NO:1 (N7), 547-565 of SEQ ID NO:1 (N8), 561-579 of SEQ ID NO:1 (N9), 589-607 of SEQ ID NO:1 (N11), 659-677 of SEQ ID NO:1 (N16), 743-761 of SEQ ID NO:1 (N22), 785-803 of SEQ ID NO:1 (N25), 813-831 of SEQ ID NO:1 (N27), 827-845 of SEQ ID NO:1 (N28), 869-887 of SEQ ID NO:1 (C2), 883-901 of SEQ ID NO:1 (C3), 911-929 of SEQ ID NO:1 (C6), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1121-1139 of SEQ ID NO:1 (C20), 1135-1153 of SEQ ID NO:1 (C21), 1177-1195 of SEQ ID NO:1 (C24), 1261-1279 of SEQ ID NO:1 (C30) or 1275-1296 of SEQ ID NO:1 (C31), or a conservative variant thereof. In another aspect of this embodiment, such a BoNT/A peptide is selected from one of the following amino acid sequences: 463-481 of SEQ ID NO:1 (N2), 533-551 of SEQ ID NO:1 (N7), 547-565 of SEQ ID NO:1 (N8), 561-579 of SEQ ID NO:1 (N9), 659-677 of SEQ ID NO:1 (N16), 743-761 of SEQ ID NO:1 (N22), 785-803 of SEQ ID NO:1 (N25), 813-831 of SEQ ID NO:1 (N27), 883-901 of SEQ ID NO:1 (C3), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (Cl 5), 1121-1139 of SEQ ID NO:1 (C20) or 1177-1195 of SEQ ID NO:1 (C24) or 1275-1296 of SEQ ID NO: 1 (C31), or a conservative variant thereof. In another aspect of this embodiment, such a BoNT/A peptide is selected from one of the following amino acid sequences: 533-551 of SEQ ID NO: 1 (N7), 547-565 of SEQ ID NO: 1 (N8), 743-761 of SEQ ID NO: 1 (N22), 785-803 of SEQ ID NO: 1 (N25), 813-831 of SEQ ID NO: 1 (N27), 995-1013 of SEQ ID NO: 1 (C11); 1051-1069 of SEQ ID NO: 1 (C15), 1177-1195 of SEQ ID NO: 1 (C24), or 1275-1296 of SEQ ID NO: 1 (C31), or a conservative variant thereof.

In yet another aspect of this embodiment, a vaccine composition comprises a BoNT/A peptide having a length of at most 60 amino acids and consisting of at least 5 contiguous amino acids selected from one of the following BoNT/A amino acid sequences: 463-481 of SEQ ID NO:1 (N2), 505-523 of SEQ ID NO:1 (N5), 519-537 of SEQ ID NO:1 (N6), 533-551 of SEQ ID NO:1 (N7), 603-621 of SEQ ID NO:1 (N12), 645-663 of SEQ ID NO:1 (N15), 659-677 of SEQ ID NO:1 (N16), 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1079-1097 of SEQ ID NO:1 (C17), 1107-1125 of SEQ ID NO:1 (C19), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28) or 1275-1296 of SEQ ID NO:1 (C31), or a conservative variant thereof. In yet another aspect of this embodiment, such a BoNT/A peptide is selected from one of the following amino acid sequences: 533-551 of SEQ ID NO:1 (N7), 659-677 of SEQ ID NO:1 (N16), 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1107-1125 of SEQ ID NO:1 (C19), 1163-1181 of SEQ ID NO:1 (C23), 1191-1209 of SEQ ID NO:1 (C25), 1233-1251 of SEQ ID NO:1 (C28) or 1275-1296 of SEQ ID NO:1 (C31), or a conservative variant thereof. In yet another aspect of this embodiment, such a BoNT/A peptide is selected from one of the following amino acid sequences: 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1079-1097 of SEQ ID NO:1 (C17), 1107-1125 of SEQ ID NO:1 (C19), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28) or 1275-1296 of SEQ ID NO:1 (C31), or a conservative variant thereof. In yet another aspect of this embodiment, such a BoNT/A peptide is selected from one of the following amino acid sequences: 729-747 of SEQ ID NO:1 (N21) 799-817 of SEQ ID NO:1 (N26), 1065-1083 of SEQ ID NO:1 (C16), 1163-1181 of SEQ ID NO:1 (C23) or 1275-1296 of SEQ ID NO:1 (C31), or a conservative variant thereof.

In yet another embodiment of the present invention, a vaccine composition comprises a BoNT/A peptide having a length of at most 60 amino acids and consisting of at least 5 contiguous amino acids selected from one of the following BoNT/A amino acid sequences: 449-467 of SEQ ID NO:1(N1), 463-481 of SEQ ID NO:1 (N2), 491-509 of SEQ ID NO:1 (N4), 505-523 of SEQ ID NO:1 (N5), 519-537 of SEQ ID NO:1 (N6), 533-551 of SEQ ID NO:1 (N7), 547-565 of SEQ ID NO:1 (N8), 561-579 of SEQ ID NO:1 (N9), 575-593 of SEQ ID NO:1 (N10), 589-607 of SEQ ID NO:1 (N11), 631-649 of SEQ ID NO:1 (N14), 645-663 of SEQ ID NO:1 (N15), 659-677 of SEQ ID NO:1 (N16), 673-691 of SEQ ID NO:1 (N17), 701-719 of SEQ ID NO:1 (N19), 715-733 of SEQ ID NO:1 (N20), 729-747 of SEQ ID NO:1 (N21), 743-761 of SEQ ID NO:1 (N22), 757-775 of SEQ ID NO:1 (N23), 771-789 of SEQ ID NO:1 (N24), 785-803 of SEQ ID NO:1 (N25), 799-817 of SEQ ID NO:1 (N26), 813-831 of SEQ ID NO:1 (N27), 827-845 of SEQ ID NO:1 (N28), 869-887 of SEQ ID NO:1 (C2), 883-901 of SEQ ID NO:1 (C3), 911-929 of SEQ ID NO:1 (C5), 925-943 of SEQ ID NO:1 (C6), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1065-1083 of SEQ ID NO:1 (C16), 1079-1097 of SEQ ID NO:1 (C17), 1107-1125 of SEQ ID NO:1 (C19), 1121-1139 of SEQ ID NO:1 (C20), 1135-1153 of SEQ ID NO:1 (C21), 1149-1167 of SEQ ID NO:1 (C22), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28), 1247-1265 of SEQ ID NO:1 (C29), 1261-1279 of SEQ ID NO:1 (C30), or 1275-1296 of SEQ ID NO:1 (C31), or a non-conservative variant thereof. In an aspect of this embodiment, such a BoNT/A peptide is selected from one of the following amino acid sequences: 463-481 of SEQ ID NO:1 (N2), 519-537 of SEQ ID NO:1 (N6), 533-551 of SEQ ID NO:1 (N7), 547-565 of SEQ ID NO:1 (N8), 561-579 of SEQ ID NO:1 (N9), 589-607 of SEQ ID NO:1 (N11), 659-677 of SEQ ID NO:1 (N16), 743-761 of SEQ ID NO:1 (N22), 785-803 of SEQ ID NO:1 (N25), 813-831 of SEQ ID NO:1 (N27), 827-845 of SEQ ID NO:1 (N28), 869-887 of SEQ ID NO:1 (C2), 883-901 of SEQ ID NO:1 (C3), 911-929 of SEQ ID NO:1 (C6), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1121-1139 of SEQ ID NO:1 (C20), 1135-1153 of SEQ ID NO:1 (C21), 1177-1195 of SEQ ID NO:1 (C24), 1261-1279 of SEQ ID NO:1 (C30) or 1275-1296 of SEQ ID NO:1 (C31), or a non-conservative variant thereof. In another aspect of this embodiment, such a BoNT/A peptide is selected from one of the following amino acid sequences: 463-481 of SEQ ID NO:1 (N2), 533-551 of SEQ ID NO:1 (N7), 547-565 of SEQ ID NO:1 (N8), 561-579 of SEQ ID NO:1 (N9), 659-677 of SEQ ID NO:1 (N16), 743-761 of SEQ ID NO:1 (N22), 785-803 of SEQ ID NO:1 (N25), 813-831 of SEQ ID NO:1 (N27), 883-901 of SEQ ID NO:1 (C3), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1121-1139 of SEQ ID NO:1 (C20) or 1177-1195 of SEQ ID NO:1 (C24) or 1275-1296 of SEQ ID NO: 1 (C31), or a non-conservative variant thereof. In another aspect of this embodiment, such a BoNT/A peptide is selected from one of the following amino acid sequences: 533-551 of SEQ ID NO: 1 (N7), 547-565 of SEQ ID NO: 1 (N8), 743-761 of SEQ ID NO: 1 (N22),.785-803 of SEQ ID NO: 1 (N25), 813-831 of SEQ ID NO: 1 (N27), 995-1013 of SEQ ID NO: 1 (C11); 1051-1069 of SEQ ID NO: 1 (C15), 1177-1195 of SEQ ID NO: 1 (C24), or 1275-1296 of SEQ ID NO: 1 (C31), or a non-conservative variant thereof.

In yet another aspect of this embodiment, a vaccine composition comprises a BoNT/A peptide having a length of at most 60 amino acids and consisting of at least 5 contiguous amino acids selected from one of the following BoNT/A amino acid sequences: 463-481 of SEQ ID NO:1 (N2), 505-523 of SEQ ID NO:1 (N5), 519-537 of SEQ ID NO:1 (N6), 533-551 of SEQ ID NO:1 (N7), 603-621 of SEQ ID NO:1 (N12), 645-663 of SEQ ID NO:1 (N15), 659-677 of SEQ ID NO:1 (N16), 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1079-1097 of SEQ ID NO:1 (C17), 1107-1125 of SEQ ID NO:1 (C19), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28) or 1275-1296 of SEQ ID NO:1 (C31), or a non-conservative variant thereof. In yet another aspect of this embodiment, such a BoNT/A peptide is selected from one of the following amino acid sequences: 533-551 of SEQ ID NO:1 (N7), 659-677 of SEQ ID NO:1 (N16), 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1107-1125 of SEQ ID NO:1 (C19), 1163-1181 of SEQ ID NO:1 (C23), 1191-1209 of SEQ ID NO:1 (C25), 1233-1251 of SEQ ID NO:1 (C28) or 1275-1296 of SEQ ID NO:1 (C31), or a non-conservative variant thereof. In yet another aspect of this embodiment, such a BoNT/A peptide is selected from one of the following amino acid sequences: 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1079-1097 of SEQ ID NO:1 (C17), 1107-1125 of SEQ ID NO:1 (C19), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28) or 1275-1296 of SEQ ID NO:1 (C31), or a non-conservative variant thereof. In yet another aspect of this embodiment, such a BoNT/A peptide is selected from one of the following amino acid sequences: 729-747 of SEQ ID NO:1 (N21) 799-817 of SEQ ID NO:1 (N26), 1065-1083 of SEQ ID NO:1 (C16), 1163-1181 of SEQ ID NO:1 (C23) or 1275-1296 of SEQ ID NO:1 (C31), or a non-conservative variant thereof.

In yet another embodiment of the present invention, a vaccine composition comprises a BoNT/A peptide having a length of at most 60 amino acids and consisting of at least 5 contiguous amino acids selected from one of the following BoNT/A amino acid sequences: 449-467 of SEQ ID NO:1(N1), 463-481 of SEQ ID NO:1 (N2), 491-509 of SEQ ID NO:1 (N4), 505-523 of SEQ ID NO:1 (N5), 519-537 of SEQ ID NO:1 (N6), 533-551 of SEQ ID NO:1 (N7), 547-565 of SEQ ID NO:1 (N8), 561-579 of SEQ ID NO:1 (N9), 575-593 of SEQ ID NO:1 (N10), 589-607 of SEQ ID NO:1 (N11), 631-649 of SEQ ID NO:1 (N14), 645-663 of SEQ ID NO:1 (N15), 659-677 of SEQ ID NO:1 (N16), 673-691 of SEQ ID NO:1 (N17), 701-719 of SEQ ID NO:1 (N19), 715-733 of SEQ ID NO:1 (N20), 729-747 of SEQ ID NO:1 (N21), 743-761 of SEQ ID NO:1 (N22), 757-775 of SEQ ID NO:1 (N23), 771-789 of SEQ ID NO:1 (N24), 785-803 of SEQ ID NO:1 (N25), 799-817 of SEQ ID NO:1 (N26), 813-831 of SEQ ID NO:1 (N27), 827-845 of SEQ ID NO:1 (N28), 869-887 of SEQ ID NO:1 (C2), 883-901 of SEQ ID NO:1 (C3), 911-929 of SEQ ID NO:1 (C5), 925-943 of SEQ ID NO:1 (C6), 939-957 of SEQ ID NO:1 (C7), 967-985 of SEQ ID NO:1 (C9), 981-999 of SEQ ID NO:1 (CO), 995-1013 of SEQ ID NO:1 (C11), 1051-1069 of SEQ ID NO:1 (C15), 1065-1083 of SEQ ID NO:1 (C16), 1079-1097 of SEQ ID NO:1 (C17), 1107-1125 of SEQ ID NO:1 (C19), 1121-1139 of SEQ ID NO:1 (C20), 1135-1153 of SEQ ID NO:1 (C21), 1149-1167 of SEQ ID NO:1 (C22), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28), 1247-1265 of SEQ ID NO:1 (C29), 1261-1279 of SEQ ID NO:1 (C30), or 1275-1296 of SEQ ID NO:1 (C31), or an immunoreactive fragment thereof, with the proviso that the BoNT/A peptide is not SEQ ID NO:2. In an aspect of this embodiment, such a BoNT/A peptide is selected from one of the following amino acid sequences: 463-481 of SEQ ID NO:1 (N2), 519-537 of SEQ ID NO:1 (N6), 533-551 of SEQ ID NO:1 (N7), 547-565 of SEQ ID NO:1 (N8), 561-579 of SEQ ID NO:1 (N9), 589-607 of SEQ ID NO:1 (N11), 659-677 of SEQ ID NO:1 (N16), 743-761 of SEQ ID NO:1 (N22), 785-803 of SEQ ID NO:1 (N25), 813-831 of SEQ ID NO:1 (N27), 827-845 of SEQ ID NO:1 (N28), 869-887 of SEQ ID NO:1 (C2), 883-901 of SEQ ID NO:1 (C3), 911-929 of SEQ ID NO:1 (C6), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1121-1139 of SEQ ID NO:1 (C20), 1135-1153 of SEQ ID NO:1 (C21), 1177-1195 of SEQ ID NO:1 (C24), 1261-1279 of SEQ ID NO:1 (C30) or 1275-1296 of SEQ ID NO:1 (C31), or an immunoreactive fragment thereof, with the proviso that the BoNT/A peptide is not SEQ ID NO:2. In another aspect of this embodiment, such a BoNT/A peptide is selected from one of the following amino acid sequences: 463-481 of SEQ ID NO:1 (N2), 533-551 of SEQ ID NO:1 (N7), 547-565 of SEQ ID NO:1 (N8), 561-579 of SEQ ID NO:1 (N9), 659-677 of SEQ ID NO:1 (N16), 743-761 of SEQ ID NO:1 (N22), 785-803 of SEQ ID NO:1 (N25), 813-831 of SEQ ID NO:1 (N27), 883-901 of SEQ ID NO:1 (C3), 939-957 of SEQ ID NO:1 (C7), 967-985 of SEQ ID NO:1 (C9), 981-999 of SEQ ID NO:1 (C10), 995-1013 of SEQ ID NO:1 (Cl 1), 1051-1069 of SEQ ID NO:1 (C15), 1121-1139 of SEQ ID NO:1 (C20) or 1177-1195 of SEQ ID NO:1 (C24) or 1275-1296 of SEQ ID NO: 1 (C31), or an immunoreactive fragment thereof, with the proviso that the BoNT/A peptide is not SEQ ID NO:2. In another aspect of this embodiment, such a BoNT/A peptide is selected from one of the following amino acid sequences: 533-551 of SEQ ID NO: 1 (N7), 547-565 of SEQ ID NO: 1 (NB), 743-761 of SEQ ID NO: 1 (N22), 785-803 of SEQ ID NO: 1 (N25), 813-831 of SEQ ID NO: 1 (N27), 995-1013 of SEQ ID NO: 1 (C11); 1051-1069 of SEQ ID NO: 1 (C15), 1177-1195 of SEQ ID NO: 1 (C24), or 1275-1296 of SEQ ID NO: 1 (C31), or an immunoreactive fragment thereof, with the proviso that the BoNT/A peptide is not SEQ ID NO:2.

In yet another aspect of this embodiment, a vaccine composition comprises a BoNT/A peptide having a length of at most 60 amino acids and consisting of at least 5 contiguous amino acids selected from one of the following BoNT/A amino acid sequences: 463-481 of SEQ ID NO:1 (N2), 505-523 of SEQ ID NO:1 (N5), 519-537 of SEQ ID NO:1 (N6), 533-551 of SEQ ID NO:1 (N7), 603-621 of SEQ ID NO:1 (N12), 645-663 of SEQ ID NO:1 (N15), 659-677 of SEQ ID NO:1 (N16), 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1079-1097 of SEQ ID NO:1 (C17), 1107-1125 of SEQ ID NO:1 (C19), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28) or 1275-1296 of SEQ ID NO:1 (C31), or an immunoreactive fragment thereof, with the proviso that the BoNT/A peptide is not SEQ ID NO:2. In yet another aspect of this embodiment, such a BoNT/A peptide is selected from one of the following amino acid sequences: 533-551 of SEQ ID NO:1 (N7), 659-677 of SEQ ID NO:1 (N16), 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1107-1125 of SEQ ID NO:1 (C19), 1163-1181 of SEQ ID NO:1 (C23), 1191-1209 of SEQ ID NO:1 (C25), 1233-1251 of SEQ ID NO:1 (C28) or 1275-1296 of SEQ ID NO:1 (C31), or an immunoreactive fragment thereof, with the proviso that the BoNT/A peptide is not SEQ ID NO:2. In yet another aspect of this embodiment, such a BoNT/A peptide is selected from one of the following amino acid sequences: 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1079-1097 of SEQ ID NO:1 (C17), 1107-1125 of SEQ ID NO:1 (C19), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28) or 1275-1296 of SEQ ID NO:1 (C31), or an immunoreactive fragment thereof, with the proviso that the BoNT/A peptide is not SEQ ID NO:2. In yet another aspect of this embodiment, such a BoNT/A peptide is selected from one of the following amino acid sequences: 729-747 of SEQ ID NO:1 (N21) 799-817 of SEQ ID NO:1 (N26), 1065-1083 of SEQ ID NO:1 (C16), 1163-1181 of SEQ ID NO:1 (C23) or 1275-1296 of SEQ ID NO:1 (C31), or an immunoreactive fragment thereof, with the proviso that the BoNT/A peptide is not SEQ ID NO:2.

In is envisioned that a BoNT/A peptide useful in a vaccine composition disclosed in the present specification can have any of a variety of lengths from at least 5 amino acids to at most 60 amino acids. Therefore, aspects of this embodiment may include a BoNT/A peptide with at least, e.g., five amino acids, six amino acids, seven amino acids, eight amino acids, nine amino acids, ten amino acids, 11 amino acids, 12 amino acids, 13 amino acids, 14 amino acids, 15 amino acids, 16 amino acids, 17 amino acids, 18 amino acids, 19 amino acids, 20 amino acids, 25 amino acids, 30 amino acids, 35 amino acids, 40 amino acids, 45 amino acids, 50 amino acids, 55 amino acids or 60 amino acids. Other aspects of this embodiment may include a BoNT/A peptide with at least, e.g., five amino acids of SEQ ID NO:1, six amino acids of SEQ ID NO:1, seven amino acids of SEQ ID NO:1, eight amino acids of SEQ ID NO:1, nine amino acids of SEQ ID NO:1, ten amino acids of SEQ ID NO:1, 11 amino acids of SEQ ID NO:1, 12 amino acids of SEQ ID NO:1, 13 amino acids of SEQ ID NO:1, 14 amino acids of SEQ ID NO:1, 15 amino acids of SEQ ID NO:1, 16 amino acids of SEQ ID NO:1, 17 amino acids of SEQ ID NO:1, 18 amino acids of SEQ ID NO:1, 19 amino acids of SEQ ID NO:1, 20 amino acids of SEQ ID NO:1, 25 amino acids of SEQ ID NO:1, 30 amino acids of SEQ ID NO:1, 35 amino acids of SEQ ID NO:1, 40 amino acids of SEQ ID NO:1, 45 amino acids of SEQ ID NO:1, 50 amino acids of SEQ ID NO:1, 55 amino acids or 60 amino acids of SEQ ID NO:1. In further embodiments, such a BoNT/A peptide of the invention may include a BoNT/A peptide with at least, e.g., five amino acids, six amino acids, seven amino acids, eight amino acids, nine amino acids, ten amino acids, 11 amino acids, 12 amino acids, 13 amino acids, 14 amino acids, 15 amino acids, 16 amino acids, 17 amino acids, 18 amino acids, 19 amino acids, 20 amino acids, 25 amino acids, 30 amino acids, 35 amino acids, 40 amino acids, 45 amino acids, 50 amino acids, 55 amino acids or 60 amino acids and consist of at least 5 contiguous amino acids selected from one of the following BoNT/A amino acid sequences: 449-467 of SEQ ID NO:1(N1), 463-481 of SEQ ID NO:1 (N2), 491-509 of SEQ ID NO:1 (N4), 505-523 of SEQ ID NO:1 (N5), 519-537 of SEQ ID NO:1 (N6), 533-551 of SEQ ID NO:1 (N7), 547-565 of SEQ ID NO:1 (N8), 561-579 of SEQ ID NO:1 (N9), 575-593 of SEQ ID NO:1 (N10), 589-607 of SEQ ID NO:1 (N11), 631-649 of SEQ ID NO:1 (N14), 645-663 of SEQ ID NO:1 (N15), 659-677 of SEQ ID NO:1 (N16), 673-691 of SEQ ID NO:1 (N17), 701-719 of SEQ ID NO:1 (N19), 715-733 of SEQ ID NO:1 (N20), 729-747 of SEQ ID NO:1 (N21), 743-761 of SEQ ID NO:1 (N22), 757-775 of SEQ ID NO:1 (N23), 771-789 of SEQ ID NO:1 (N24), 785-803 of SEQ ID NO:1 (N25), 799-817 of SEQ ID NO:1 (N26), 813-831 of SEQ ID NO:1 (N27), 827-845 of SEQ ID NO:1 (N28), 869-887 of SEQ ID NO:1 (C2), 883-901 of SEQ ID NO:1 (C3), 911-929 of SEQ ID NO:1 (C5), 925-943 of SEQ ID NO:1 (C6), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1065-1083 of SEQ ID NO:1 (C16), 1079-1097 of SEQ ID NO:1 (C17), 1107-1125 of SEQ ID NO:1 (C19), 1121-1139 of SEQ ID NO:1 (C20), 1135-1153 of SEQ ID NO:1 (C21), 1149-1167 of SEQ ID NO:1 (C22), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28), 1247-1265 of SEQ ID NO:1 (C29), 1261-1279 of SEQ ID NO:1 (C30) or 1275-1296 of SEQ ID NO:1 (C31), or a conservative variant, a non-conservative variant, or immunoreactive fragment thereof, with the proviso that the BoNT/A peptide is not SEQ ID NO:2.

In another embodiment of the present invention, a vaccine composition can comprise one BoNT/A peptide disclosed in the present specification. In another embodiment of the present invention, a vaccine composition can comprise a plurality of BoNT/A peptides disclosed in the present specification. Thus, aspects of this embodiment can include one or more BoNT/A peptides, two or more BoNT/A peptides, three or more BoNT/A peptides, four or more BoNT/A peptides, five or more BoNT/A peptides, six or more BoNT/A peptides, seven or more BoNT/A peptides, eight or more BoNT/A peptides, nine or more BoNT/A peptides, ten or more BoNT/A peptides, 15 or more BoNT/A peptides, 20 or more BoNT/A peptides, 25 or more BoNT/A peptides or 30 or more BoNT/A peptides. In other aspects of this embodiment can include one or more BoNT/A conservative variants, two or more BoNT/A conservative variants, three or more BoNT/A conservative variants, four or more BoNT/A conservative variants, five or more BoNT/A conservative variants, six or more BoNT/A conservative variants, seven or more BoNT/A conservative variants, eight or more BoNT/A conservative variants, nine or more BoNT/A conservative variants, ten or more BoNT/A conservative variants, 15 or more BoNT/A conservative variants, 20 or more BoNT/A conservative variants, 25 or more BoNT/A conservative variants or 30 or more BoNT/A conservative variants. In further aspects of this embodiment can include one or more BoNT/A non-conservative variants, two or more BoNT/A non-conservative variants, three or more BoNT/A non-conservative variants, four or more BoNT/A non-conservative variants, five or more BoNT/A non-conservative variants, six or more BoNT/A non-conservative variants, seven or more BoNT/A non-conservative variants, eight or more BoNT/A non-conservative variants, nine or more BoNT/A non-conservative variants, ten or more BoNT/A non-conservative variants, 15 or more BoNT/A non-conservative variants, 20 or more BoNT/A non-conservative variants, 25 or more BoNT/A non-conservative variants or 30 or more BoNT/A non-conservative variants. In still other aspects of this embodiment can include one or more BoNT/A immunoreactive fragments, two or more BoNT/A immunoreactive fragments, three or more BoNT/A immunoreactive fragments, four or more BoNT/A immunoreactive fragments, five or more BoNT/A immunoreactive fragments, six or more BoNT/A immunoreactive fragments, seven or more BoNT/A immunoreactive fragments, eight or more BoNT/A immunoreactive fragments, nine or more BoNT/A peptides, ten or more BoNT/A immunoreactive fragments, 15 or more BoNT/A immunoreactive fragments, 20 or more BoNT/A immunoreactive fragments, 25 or more BoNT/A immunoreactive fragments or 30 or more BoNT/A immunoreactive fragments. BoNT/A peptides disclosed in the present specification useful for a vaccine composition can be selected, for example, depending on immunological factors, such as potency of the peptide in inducing an immune response, and technical factors, such as chemical synthesis yields. It is also understood that the two or more BoNT/A peptides can be provided separately or as part of a compound molecule such as a chimeric peptide or heterologous protein.

In an aspect of this embodiment, a vaccine composition comprises two or more of the following amino acid sequences: 533-551 of SEQ ID NO:1 (N7), 547-565 of SEQ ID NO:1 (N8), 743-761 of SEQ ID NO:1 (N22), 785-803 of SEQ ID NO:1 (N25), 813-831 of SEQ ID NO:1 (N27), 995-1013 of SEQ ID NO:1 (C11); 1051-1069 of SEQ ID NO:1 (C15), 1177-1195 of SEQ ID NO:1 (C24), and 1275-1296 of SEQ ID NO:1 (C31), or a conservative variant, a non-conservative variant or an immunoreactive fragment thereof, with the proviso that the BoNT/A peptide is not SEQ ID NO:2. In another aspect of this embodiment, one of the selected amino acid sequence is 533-551 of SEQ ID NO:1 (N8) or a conservative variant, a non-conservative variant or an immunoreactive fragment thereof, with the proviso that the BoNT/A peptide is not SEQ ID NO:2. In yet another aspect of this embodiment, the following two amino acid sequences are selected: 533-551 of SEQ ID NO:1 (N8) and 981-999 of SEQ ID NO:1 (C10), or a conservative variant, a non-conservative variant or an immunoreactive fragment thereof, with the proviso that the BoNT/A peptide is not SEQ ID NO:2. In yet another aspect of this embodiment, the following three amino acid sequences are selected: 533-551 of SEQ ID NO:1 (N8), 981-999 of SEQ ID NO:1 (C10) and 1051-1069 of SEQ ID NO:1 (C15), or a conservative variant, a non-conservative variant or an immunoreactive fragment thereof, with the proviso that the BoNT/A peptide is not SEQ ID NO:2. In a further aspect of this embodiment, one of the amino acid sequences selected is 785-803 of SEQ ID NO:1 (N25) or a conservative variant, a non-conservative variant or an immunoreactive fragment thereof, with the proviso that the BoNT/A peptide is not SEQ ID NO:2. In a still further aspect of this embodiment, the following two amino acid sequences are selected: 785-803 of SEQ ID NO:1 (N25) and 981-999 of SEQ ID NO:1 (C10), or a conservative variant, a non-conservative variant or an immunoreactive fragment thereof, with the proviso that the BoNT/A peptide is not SEQ ID NO:2. In a still further aspect of this embodiment, the following three amino acid sequences are selected: 785-803 of SEQ ID NO:1 (N25), 981-999 of SEQ ID NO:1 (C10) and 1051-1069 of SEQ ID NO:1 (C15), or a conservative variant, a non-conservative variant or an immunoreactive fragment thereof, with the proviso that the BoNT/A peptide is not SEQ ID NO:2. In an additional aspect of this embodiment, one of the amino acid sequences selected is 813-831 of SEQ ID NO:1 (N27) or a conservative variant, a non-conservative variant or an immunoreactive fragment thereof, with the proviso that the BoNT/A peptide is not SEQ ID NO:2. In another additional aspect of this embodiment, the following two amino acid sequences are selected: 813-831 of SEQ ID NO:1 (N27) and 981-999 of SEQ ID NO:1 (C10), or a conservative variant, a non-conservative variant or an immunoreactive fragment thereof, with the proviso that the BoNT/A peptide is not SEQ ID NO:2. In another additional aspect of this embodiment, the following three amino acid sequences are selected: 813-831 of SEQ ID NO:1 (N27), 981-999 of SEQ ID NO:1 (C10) and 1051-1069 of SEQ ID NO:1 (C15), or a conservative variant, a non-conservative variant or an immunoreactive fragment thereof, with the proviso that the BoNT/A peptide is not SEQ ID NO:2.

In an aspect of this embodiment, a vaccine composition comprises two or more of the following amino acid sequences: 659-677 of SEQ ID NO:1 (N16), 729-747 of SEQ ID NO:1 (N21), 799-817 of SEQ ID NO:1 (N26), 1065-1083 of SEQ ID NO:1 (C16), 1163-1181 of SEQ ID NO:1 (C23), and 1275-1296 of SEQ ID NO:1 (C31), or a conservative variant, a non-conservative variant or an immunoreactive fragment thereof, with the proviso that the BoNT/A peptide is not SEQ ID NO:2. In another aspect of this embodiment, one of the amino acid sequences selected is 1065-1083 of SEQ ID NO:1 (C16) or a conservative variant, a non-conservative variant or an immunoreactive fragment thereof, with the proviso that the BoNT/A peptide is not SEQ ID NO:2. In yet another aspect of this embodiment, the following two amino acid sequences are selected: 1065-1083 of SEQ ID NO:1 (C16) and 1163-1181 of SEQ ID NO:1 (C23), or a conservative variant, a non-conservative variant or an immunoreactive fragment thereof, with the proviso that the BoNT/A peptide is not SEQ ID NO:2. In yet another aspect of this embodiment, the following three amino acid sequences are selected: 1065-1083 of SEQ ID NO:1 (C16), 1163-1181 of SEQ ID NO:1 (C23) and 1275-1296 of SEQ ID NO:1 (C31), or a conservative variant, a non-conservative variant or an immunoreactive fragment thereof, with the proviso that the BoNT/A peptide is not SEQ ID NO:2. In a further aspect of this embodiment, one of the amino acid sequences selected is 799-817 of SEQ ID NO:1 (N26) or a conservative variant, a non-conservative variant or an immunoreactive fragment thereof, with the proviso that the BoNT/A peptide is not SEQ ID NO:2. In a still further aspect of this embodiment, the following two amino acid sequences are selected: 799-817 of SEQ ID NO:1 (N26) and 1065-1083 of SEQ ID NO:1 (C16), or a conservative variant, a non-conservative variant or an immunoreactive fragment thereof, with the proviso that the BoNT/A peptide is not SEQ ID NO:2. In a still further aspect of this embodiment, the following three amino acid sequences are selected: 799-817 of SEQ ID NO:1 (N26), 1065-1083 of SEQ ID NO:1 (C16) and 1163-1181 of SEQ ID NO:1 (C23), or a conservative variant, a non-conservative variant or an immunoreactive fragment thereof, with the proviso that the BoNT/A peptide is not SEQ ID NO:2. In an additional aspect of this embodiment, one of the amino acid sequences selected is 729-747 of SEQ ID NO:1 (N21) or a conservative variant, a non-conservative variant or an immunoreactive fragment thereof, with the proviso that the BoNT/A peptide is not SEQ ID NO:2. In another additional aspect of this embodiment, the following two amino acid sequences are selected: 729-747 of SEQ ID NO:1 (N21) and 1065-1083 of SEQ ID NO:1 (C16), or a conservative variant, a non-conservative variant or an immunoreactive fragment thereof, with the proviso that the BoNT/A peptide is not SEQ ID NO:2. In another additional aspect of this embodiment, the following three amino acid sequences are selected: 729-747 of SEQ ID NO:1 (N21), 1065-1083 of SEQ ID NO:1 (C16) and 1163-1181 of SEQ ID NO:1 (C23), or a conservative variant, a non-conservative variant or an immunoreactive fragment thereof, with the proviso that the BoNT/A peptide is not SEQ ID NO:2.

It is also envisioned that any and all combinations of BoNT/A peptides disclosed in the specification, including, e.g., BoNT/A peptides of SEQ ID NO:1, BoNT/A conservative variants, BoNT/A non-conservative variants and BoNT/A immunoreactive fragments, can be used in a vaccine composition. Thus, aspects of this embodiment include one or more BoNT/A peptides comprising one or more BoNT/A peptides of SEQ ID NO: 1 and one or more BoNT/A conservative variants; one or more BoNT/A peptides of SEQ ID NO: 1 and one or more BoNT/A non-conservative variants; one or more BoNT/A peptides of SEQ ID NO: 1 and one or more BoNT/A immunoreactive fragments; one or more BoNT/A conservative variants and one or more BoNT/A non-conservative variants; one or more BoNT/A conservative variants and one or more BoNT/A immunoreactive fragments; one or more BoNT/A non-conservative variants and one or more BoNT/A immunoreactive fragments; one or more BoNT/A peptides of SEQ ID NO: 1, one or more BoNT/A conservative variants and one or more BoNT/A non-conservative variants; one or more BoNT/A peptides of SEQ ID NO: 1, one or more BoNT/A conservative variants and one or more BoNT/A immunoreactive fragments; one or more BoNT/A peptides of SEQ ID NO: 1, one or more BoNT/A non-conservative variants and one or more BoNT/A immunoreactive fragments; one or more BoNT/A conservative variants, one or more BoNT/A non-conservative variants and one or more BoNT/A immunoreactive fragments; or one or more BoNT/A peptides of SEQ ID NO: 1, one or more BoNT/A conservative variants, one or more BoNT/A non-conservative variants and one or more BoNT/A immunoreactive fragments.

In yet another embodiment of the present invention, a vaccine composition can optionally comprises one or more carriers. The main objective of these carriers is to enhance the immunogenicity of an antigen, a hapten, or any other antigenic compound that is immunogenic, non-immunogenic, or weakly immunogenic when not associated with the carrier. The use of carriers in therapeutic compositions of the vaccine type is well known, see, e.g., David W. Waggoner, Jr. et al., Immunogenicity-enhancing carriers and compositions thereof and methods of using the same, U.S. Patent Publication No. 20040057958 (Mar. 25, 2004), which is hereby incorporated by reference in its entirety.

In yet another embodiment of the present invention, a vaccine composition also optionally comprises one or more adjuvants. The term “adjuvant”, as used herein, means any substance or mixture of substances that increases or diversifies the immune response to an antigenic compound. An adjuvant can, for example, serve to reduce the number of immunizations or the amount of antigen required for protective immunization. In certain embodiments, an vaccine composition optionally comprises one or more adjuvants. The use of adjuvants in therapeutic compositions of the vaccine type is well known. The main objective of these adjuvants is to allow an increase in the immune response. These adjuvants are diverse in nature. They may, for example, consist of liposomes, oily phases, including, without limitation, the Freund type of adjuvants, such as, e.g., Freund's complete adjuvant (FCA); Freund's incomplete adjuvant (FIA); sapogenin glycosides, such as, e.g., saponins; ; carbopol; N-acetylmuramyl-L-alanyl-D-isoglutamine (commonly known as muramyl dipeptide or “MDP”); and lipopolysaccharide (LPS). Such adjuvants are generally used in the form of an emulsion with an aqueous phase, or, more commonly, may consist of water-insoluble inorganic salts. These inorganic salts may consist, for example, of aluminum hydroxide, zinc sulfate, colloidal iron hydroxide, calcium phosphate or calcium chloride. Aluminum hydroxide (Al(OH).sub.3) is a commonly used adjuvant. Currently, the only FDA-approved adjuvant for use in humans is aluminum salts (Alum) which are used to “depot” antigens by precipitation of the antigens. Adjuvants provided above are merely exemplary. In fact, any adjuvant may be used in the immunogenic composition of the present invention as long as the adjuvant satisfies the requisite characteristics that are necessary for practicing the present invention. As indicated above, the carrier of the compositions of the present invention itself may act as an adjuvant. Specific adjuvants and methods of making and using are are described in, e.g., Gupta et al. Vaccine, 11: 993-306, 1993; Arnon, R. (Ed.) Synthetic Vaccines 1:83-92, CRC Press, Inc., Boca Raton, Fla., 1987; and David W. Waggoner, Jr. et al., Immunogenicity-Enhancing Carriers and Compositions Thereof and Methods of Using the Same, U.S. Patent Publication No. 20040057958 (Mar. 25, 2004), which are hereby incorporated by reference in their entirety. Additional adjuvants include any compound described in Chapter 7 (pp 141-227) of “Vaccine Design, The Subunit and Adjuvant Approach” (eds. Powell, M. F. and Newman, M. J.) Pharmaceutical Biotechnology, Volume 6, Plenum Press (New York). Examples from this compendium include Muramyl Dipeptide (MDP) and Montanide 720. Molecules such as Poly Inosine:Cytosine (Poly I:C) or plasmid DNA containing CpG motifs can also be administered as adjuvants in combination with antigens encapsulated in microparticles. In another example, the adjuvant is an agent that facilitates entry of the antigenic compound into the cytoplasm of a cell such as listeriolysin, streptolysin or a mixture thereof.

In yet another embodiment of the present invention, a vaccine composition can includes a BoNT/A peptide which is, for example, conjugated to, or expressed as, a fusion protein with another molecule. The molecule selected for fusion to a BoNT/A peptide will depend on the particular design of the vaccine. Non-limiting examples of BoNT/A fusion proteins useful in the invention include fusions with molecules that increase immune response against the BoNT/A peptide, such as cholera enterotoxin A2 and other peptides against which an immune response is desired, such as another BONT peptide. In one embodiment, a vaccine of the invention contains a BoNT/A peptide fused to a peptide or protein adjuvant.

V. BoNT/A Antibody Compositions

The present invention further provides an antibody composition having selectivity for an epitope contained within a BoNT/A peptide disclosed in the present specification. In one embodiment of the present invention, an antibody composition selectively binds to an eptitope contained within a BoNT/A peptide having a length of at most 60 amino acids and consisting of at least 5 contiguous amino acids selected from one of the following BoNT/A amino acid sequences: 449-467 of SEQ ID NO:1(N1), 463-481 of SEQ ID NO:1 (N2), 491-509 of SEQ ID NO:1 (N4), 505-523 of SEQ ID NO:1 (N5), 519-537 of SEQ ID NO:1 (N6), 533-551 of SEQ ID NO:1 (N7), 547-565 of SEQ ID NO:1 (N8), 561-579 of SEQ ID NO:1 (N9), 575-593 of SEQ ID NO:1 (N10), 589-607 of SEQ ID NO:1 (N11), 631-649 of SEQ ID NO:1 (N14), 645-663 of SEQ ID NO:1 (N15), 659-677 of SEQ ID NO:1 (N16), 673-691 of SEQ ID NO:1 (N17), 701-719 of SEQ ID NO:1 (N19), 715-733 of SEQ ID NO:1 (N20), 729-747 of SEQ ID NO:1 (N21), 743-761 of SEQ ID NO:1 (N22), 757-775 of SEQ ID NO:1 (N23), 771-789 of SEQ ID NO:1 (N24), 785-803 of SEQ ID NO:1 (N25), 799-817 of SEQ ID NO:1 (N26), 813-831 of SEQ ID NO:1 (N27), 827-845 of SEQ ID NO:1 (N28), 869-887 of SEQ ID NO:1 (C2), 883-901 of SEQ ID NO:1 (C3), 911-929 of SEQ ID NO:1 (C5), 925-943 of SEQ ID NO:1 (C6), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1065-1083 of SEQ ID NO:1 (C16), 1079-1097 of SEQ ID NO:1 (C17), 1107-1125 of SEQ ID NO:1 (C19), 1121-1139 of SEQ ID NO:1 (C20), 1135-1153 of SEQ ID NO:1 (C21), 1149-1167 of SEQ ID NO:1 (C22), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28), 1247-1265 of SEQ ID NO:1 (C29), 1261-1279 of SEQ ID NO:1 (C30), or 1275-1296 of SEQ ID NO:1 (C31), or a conservative variant or immunoreactive fragment thereof, with the proviso that the BoNT/A peptide is not SEQ ID NO:2.

In another embodiment of the present invention, an antibody composition selectively binds to an eptitope contained within a BoNT/A peptide having a length of at most 60 amino acids and consisting of at least 5 contiguous amino acids selected from one of the following BoNT/A amino acid sequences: 449-467 of SEQ ID NO:1(N1), 463-481 of SEQ ID NO:1 (N2), 491-509 of SEQ ID NO:1 (N4), 505-523 of SEQ ID NO:1 (N5), 519-537 of SEQ ID NO:1 (N6), 533-551 of SEQ ID NO:1 (N7), 547-565 of SEQ ID NO:1 (N8), 561-579 of SEQ ID NO:1 (N9), 575-593 of SEQ ID NO:1 (N10), 589-607 of SEQ ID NO:1 (N11), 631-649 of SEQ ID NO:1 (N14), 645-663 of SEQ ID NO:1 (N15), 659-677 of SEQ ID NO:1 (N16), 673-691 of SEQ ID NO:1 (N17), 701-719 of SEQ ID NO:1 (N19), 715-733 of SEQ ID NO:1 (N20), 729-747 of SEQ ID NO:1 (N21), 743-761 of SEQ ID NO:1 (N22), 757-775 of SEQ ID NO:1 (N23), 771-789 of SEQ ID NO:1 (N24), 785-803 of SEQ ID NO:1 (N25), 799-817 of SEQ ID NO:1 (N26), 813-831 of SEQ ID NO:1 (N27), 827-845 of SEQ ID NO:1 (N28), 869-887 of SEQ ID NO:1 (C2), 883-901 of SEQ ID NO:1 (C3), 911-929 of SEQ ID NO:1 (C5), 925-943 of SEQ ID NO:1 (C6), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1065-1083 of SEQ ID NO:1 (C16), 1079-1097 of SEQ ID NO:1 (C17), 1107-1125 of SEQ ID NO:1 (C19), 1121-1139 of SEQ ID NO:1 (C20), 1135-1153 of SEQ ID NO:1 (C21), 1149-1167 of SEQ ID NO:1 (C22), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28), 1247-1265 of SEQ ID NO:1 (C29), 1261-1279 of SEQ ID NO:1 (C30), or 1275-1296 of SEQ ID NO:1 (C31). In an aspect of this embodiment, such a BoNT/A peptide is selected from one of the following amino acid sequences: 463-481 of SEQ ID NO:1 (N2), 519-537 of SEQ ID NO:1 (N6), 533-551 of SEQ ID NO:1 (N7), 547-565 of SEQ ID NO:1 (N8), 561-579 of SEQ ID NO:1 (N9), 589-607 of SEQ ID NO:1 (N11), 659-677 of SEQ ID NO:1 (N16), 743-761 of SEQ ID NO:1 (N22), 785-803 of SEQ ID NO:1 (N25), 813-831 of SEQ ID NO:1 (N27), 827-845 of SEQ ID NO:1 (N28), 869-887 of SEQ ID NO:1 (C2), 883-901 of SEQ ID NO:1 (C3), 911-929 of SEQ ID NO:1 (C6), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1121-1139 of SEQ ID NO:1 (C20), 1135-1153 of SEQ ID NO:1 (C21), 1177-1195 of SEQ ID NO:1 (C24), 1261-1279 of SEQ ID NO:1 (C30) or 1275-1296 of SEQ ID NO:1 (C31). In another aspect of this embodiment, such a BoNT/A peptide is selected from one of the following amino acid sequences: 463-481 of SEQ ID NO:1 (N2), 533-551 of SEQ ID NO:1 (N7), 547-565 of SEQ ID NO:1 (N8), 561-579 of SEQ ID NO:1 (N9), 659-677 of SEQ ID NO:1 (N16), 743-761 of SEQ ID NO:1 (N22), 785-803 of SEQ ID NO:1 (N25), 813-831 of SEQ ID NO:1 (N27), 883-901 of SEQ ID NO:1 (C3), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1121-1139 of SEQ ID NO:1 (C20) or 1177-1195 of SEQ ID NO:1 (C24) or 1275-1296 of SEQ ID NO: 1 (C31). In another aspect of this embodiment, such a BoNT/A peptide is selected from one of the following amino acid sequences: 533-551 of SEQ ID NO: 1 (N7), 547-565 of SEQ ID NO: 1 (N8), 743-761 of SEQ ID NO: 1 (N22), 785-803 of SEQ ID NO: 1 (N25), 813-831 of SEQ ID NO: 1 (N27), 995-1013 of SEQ ID NO: 1 (C11); 1051-1069 of SEQ ID NO: 1 (C15), 1177-1195 of SEQ ID NO: 1 (C24), or 1275-1296 of SEQ ID NO: 1 (C31).

In yet another aspect of this embodiment, an antibody composition selectively binds to an eptitope contained within a BoNT/A peptide having a length of at most 60 amino acids and consisting of at least 5 contiguous amino acids selected from one of the following BoNT/A amino acid sequences: 463-481 of SEQ ID NO:1 (N2), 505-523 of SEQ ID NO:1 (N5), 519-537 of SEQ ID NO:1 (N6), 533-551 of SEQ ID NO:1 (N7), 603-621 of SEQ ID NO:1 (N12), 645-663 of SEQ ID NO:1 (N15), 659-677 of SEQ ID NO:1 (N16), 701-719 of SEQ ID NO:1 (N19), 729-747 of SEQ ID NO:1 (N21), 757-775 of SEQ ID NO:1 (N23), 799-817 of SEQ ID NO:1 (N260, 1065-1083 of SEQ ID NO:1 (C16), 1079-1097 of SEQ ID NO:1 (C17), 1107-1125 of SEQ ID NO:1 (C19), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28) or 1275-1296 of SEQ ID NO:1 (C31). In yet another aspect of this embodiment, such a BoNT/A peptide is selected from one of the following amino acid sequences: 533-551 of SEQ ID NO:1 (N7), 659-677 of SEQ ID NO:1 (N16), 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1107-1125 of SEQ ID NO:1 (C19), 1163-1181 of SEQ ID NO:1 (C23), 1191-1209 of SEQ ID NO:1 (C25), 1233-1251 of SEQ ID NO:1 (C28) or 1275-1296 of SEQ ID NO:1 (C31). In yet another aspect of this embodiment, such a BoNT/A peptide is selected from one of the following amino acid sequences: 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1079-1097 of SEQ ID NO:1 (C17), 1107-1125 of SEQ ID NO:1 (C19), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28) or 1275-1296 of SEQ ID NO:1 (C31). In yet another aspect of this embodiment, such a BoNT/A peptide is selected from one of the following amino acid sequences: 729-747 of SEQ ID NO:1 (N21) 799-817 of SEQ ID NO:1 (N26), 1065-1083 of SEQ ID NO:1 (C16), 1163-1181 of SEQ ID NO:1 (C23) or 1275-1296 of SEQ ID NO:1 (C31).

In yet another embodiment of the present invention, an antibody composition selectively binds to an eptitope contained within a BoNT/A peptide selected from one of the following BoNT/A amino acid sequences: 449-467 of SEQ ID NO:1(N1), 463-481 of SEQ ID NO:1 (N2), 491-509 of SEQ ID NO:1 (N4), 505-523 of SEQ ID NO:1 (N5), 519-537 of SEQ ID NO:1 (N6), 533-551 of SEQ ID NO:1 (N7), 547-565 of SEQ ID NO:1 (N8), 561-579 of SEQ ID NO:1 (N9), 575-593 of SEQ ID NO:1 (N10), 589-607 of SEQ ID NO:1 (N11), 631-649 of SEQ ID NO:1 (N14), 645-663 of SEQ ID NO:1 (N15), 659-677 of SEQ ID NO:1 (N16), 673-691 of SEQ ID NO:1 (N17), 701-719 of SEQ ID NO:1 (N19), 715-733 of SEQ ID NO:1 (N20), 729-747 of SEQ ID NO:1 (N21), 743-761 of SEQ ID NO:1 (N22), 757-775 of SEQ ID NO:1 (N23), 771-789 of SEQ ID NO:1 (N24), 785-803 of SEQ ID NO:1 (N25), 799-817 of SEQ ID NO:1 (N26), 813-831 of SEQ ID NO:1 (N27), 827-845 of SEQ ID NO:1 (N28), 869-887 of SEQ ID NO:1 (C2), 883-901 of SEQ ID NO:1 (C3), 911-929 of SEQ ID NO:1 (C5), 925-943 of SEQ ID NO:1 (C6), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1065-1083 of SEQ ID NO:1 (C16), 1079-1097 of SEQ ID NO:1 (C17), 1107-1125 of SEQ ID NO:1 (C19), 1121-1139 of SEQ ID NO:1 (C20), 1135-1153 of SEQ ID NO:1 (C21), 1149-1167 of SEQ ID NO:1 (C22), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28), 1247-1265 of SEQ ID NO:1 (C29), 1261-1279 of SEQ ID NO:1 (C30), or 1275-1296 of SEQ ID NO:1 (C31). In an aspect of this embodiment, such a BoNT/A peptide is selected from one of the following amino acid sequences: 463-481 of SEQ ID NO:1 (N2), 519-537 of SEQ ID NO:1 (N6), 533-551 of SEQ ID NO:1 (N7), 547-565 of SEQ ID NO:1 (N8), 561-579 of SEQ ID NO:1 (N9), 589-607 of SEQ ID NO:1 (N11), 659-677 of SEQ ID NO:1 (N16), 743-761 of SEQ ID NO:1 (N22), 785-803 of SEQ ID NO:1 (N25), 813-831 of SEQ ID NO:1 (N27), 827-845 of SEQ ID NO:1 (N28), 869-887 of SEQ ID NO:1 (C2), 883-901 of SEQ ID NO:1 (C3), 911-929 of SEQ ID NO:1 (C6), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1121-1139 of SEQ ID NO:1 (C20), 1135-1153 of SEQ ID NO:1 (C21), 1177-1195 of SEQ ID NO:1 (C24), 1261-1279 of SEQ ID NO:1 (C30) or 1275-1296 of SEQ ID NO:1 (C31). In another aspect of this embodiment, such a BoNT/A peptide is selected from one of the following amino acid sequences: 463-481 of SEQ ID NO:1 (N2), 533-551 of SEQ ID NO:1 (N7), 547-565 of SEQ ID NO:1 (N8), 561-579 of SEQ ID NO:1 (N9), 659-677 of SEQ ID NO:1 (N16), 743-761 of SEQ ID NO:1 (N22), 785-803 of SEQ ID NO:1 (N25), 813-831 of SEQ ID NO:1 (N27), 883-901 of SEQ ID NO:1 (C3), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1121-1139 of SEQ ID NO:1 (C20) or 1177-1195 of SEQ ID NO:1 (C24) or 1275-1296 of SEQ ID NO: 1 (C31). In another aspect of this embodiment, such a BoNT/A peptide is selected from one of the following amino acid sequences: 533-551 of SEQ ID NO: 1 (N7), 547-565 of SEQ ID NO: 1 (N8), 743-761 of SEQ ID NO: 1 (N22), 785-803 of SEQ ID NO: 1 (N25), 813-831 of SEQ ID NO: 1 (N27), 995-1013 of SEQ ID NO: 1 (C11); 1051-1069 of SEQ ID NO: 1 (C15),1177-1195 of SEQ ID NO: 1 (C24), or 1275-1296 of SEQ ID NO: 1 (C31).

In yet another aspect of this embodiment, an antibody composition selectively binds to an eptitope contained within a BoNT/A peptide selected from one of the following BoNT/A amino acid sequences: 463-481 of SEQ ID NO:1 (N2), 505-523 of SEQ ID NO:1 (N5), 519-537 of SEQ ID NO:1 (N6), 533-551 of SEQ ID NO:1 (N7), 603-621 of SEQ ID NO:1 (N1 2), 645-663 of SEQ ID NO:1 (N15), 659-677 of SEQ ID NO:1 (N16), 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1079-1097 of SEQ ID NO:1 (C17), 1107-1125 of SEQ ID NO:1 (C19), 1163-1181 of SEQ ID NO:1 (C23),1177-1195 of SEQ ID NO:1 (C24), 1191-1209 of SEQ ID NO:1 (C25), 1233-1251 of SEQ ID NO:1 (C28) or 1275-1296 of SEQ ID NO:1 (C31). In yet another aspect of this embodiment, such a BoNT/A peptide is selected from one of the following amino acid sequences: 533-551 of SEQ ID NO:1 (N7), 659-677 of SEQ ID NO:1 (N16), 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1107-1125 of SEQ ID NO:1 (C19), 1163-1181 of SEQ ID NO:1 (C23), 1191-1209 of SEQ ID NO:1 (C25), 1233-1251 of SEQ ID NO:1 (C28) or 1275-1296 of SEQ ID NO:1 (C31). In yet another aspect of this embodiment, such a BoNT/A peptide is selected from one of the following amino acid sequences: 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1079-1097 of SEQ ID NO:1 (C17), 1107-1125 of SEQ ID NO:1 (C19), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28) or 1275-1296 of SEQ ID NO:1 (C31). In yet another aspect of this embodiment, such a BoNT/A peptide is selected from one of the following amino acid sequences: 729-747 of SEQ ID NO:1 (N21) 799-817 of SEQ ID NO:l (N26), 1065-1083 of SEQ ID NO:1 (C16), 1163-1181 of SEQ ID NO:1 (C23) or 1275-1296 of SEQ ID NO:1 (C31).

In yet another embodiment of the present invention, an antibody composition selectively binds to an eptitope contained within a BoNT/A peptide having a length of at most 60 amino acids and consisting of at least 5 contiguous amino acids selected from one of the following BoNT/A amino acid sequences: 449-467 of SEQ ID NO:1(N1), 463-481 of SEQ ID NO:1 (N2), 491-509 of SEQ ID NO:l (N4), 505-523 of SEQ ID NO:1 (N5), 519-537 of SEQ ID NO:1 (N6), 533-551 of SEQ ID NO:1 (N7), 547-565 of SEQ ID NO:1 (N8), 561-579 of SEQ ID NO:1 (N9), 575-593 of SEQ ID NO:1 (N10), 589-607 of SEQ ID NO:1 (N11), 631-649 of SEQ ID NO:1 (N14), 645-663 of SEQ ID NO:1 (N15), 659-677 of SEQ ID NO:1 (N16), 673-691 of SEQ ID NO:1 (N17), 701-719 of SEQ ID NO:1 (N19), 715-733 of SEQ ID NO:1 (N20), 729-747 of SEQ ID NO:1 (N21), 743-761 of SEQ ID NO:1 (N22), 757-775 of SEQ ID NO:1 (N23), 771-789 of SEQ ID NO:1 (N24), 785-803 of SEQ ID NO:1 (N25), 799-817 of SEQ ID NO:1 (N26), 813-831 of SEQ ID NO:1 (N27), 827-845 of SEQ ID NO:1 (N28), 869-887 of SEQ ID NO:1 (C2), 883-901 of SEQ ID NO:1 (C3), 911-929 of SEQ ID NO:1 (C5), 925-943 of SEQ ID NO:1 (C6), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1065-1083 of SEQ ID NO:1 (C16), 1079-1097 of SEQ ID NO:1 (C17), 1107-1125 of SEQ ID NO:1 (C19), 1121-1139 of SEQ ID NO:1 (C20), 1135-1153 of SEQ ID NO:1 (C21),1149-1167 of SEQ ID NO:1 (C22), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28), 1247-1265 of SEQ ID NO:1 (C29), 1261-1279 of SEQ ID NO:1 (C30), or 1275-1296 of SEQ ID NO:1 (C31), or a conservative variant thereof. In an aspect of this embodiment, such a BoNT/A peptide is selected from one of the following amino acid sequences: 463-481 of SEQ ID NO:1 (N2), 519-537 of SEQ ID NO:1 (N6), 533-551 of SEQ ID NO:1 (N7),547-565 of SEQ ID NO:1 (N8),561-579 of SEQ ID NO:1 (N9), 589-607 of SEQ ID NO:1 (N11), 659-677 of SEQ ID NO:1 (N16), 743-761 of SEQ ID NO:1 (N22), 785-803 of SEQ ID NO:1 (N25), 813-831 of SEQ ID NO:1 (N27), 827-845 of SEQ ID NO:1 (N28), 869-887 of SEQ ID NO:1 (C2), 883-901 of SEQ ID NO:1 (C3), 911-929 of SEQ ID NO:1 (C6), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1121-1139 of SEQ ID NO:1 (C20), 1135-1153 of SEQ ID NO:1 (C21), 1177-1195 of SEQ ID NO:1 (C24), 1261-1279 of SEQ ID NO:1 (C30) or 1275-1296 of SEQ ID NO:1 (C31), or a conservative variant thereof. In another aspect of this embodiment, such a BoNT/A peptide is selected from one of the following amino acid sequences: 463-481 of SEQ ID NO:1 (N2), 533-551 of SEQ ID NO:1 (N7), 547-565 of SEQ ID NO:1 (N8), 561-579 of SEQ ID NO:1 (N9), 659-677 of SEQ ID NO:1 (N1 6), 743-761 of SEQ ID NO:1 (N22), 785-803 of SEQ ID NO:1 (N25), 813-831 of SEQ ID NO:1 (N27), 883-901 of SEQ ID NO:1 (C3), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1121-1139 of SEQ ID NO:1 (C20) or 1177-1195 of SEQ ID NO:1 (C24) or 1275-1296 of SEQ ID NO: 1 (C31), or a conservative variant thereof. In another aspect of this embodiment, such a BoNT/A peptide is selected from one of the following amino acid sequences: 533-551 of SEQ ID NO: 1 (N7), 547-565 of SEQ ID NO: 1 (N8), 743-761 of SEQ ID NO: 1 (N22), 785-803 of SEQ ID NO: 1 (N25), 813-831 of SEQ ID NO: 1 (N27), 995-1013 of SEQ ID NO: 1 (C11); 1051-1069 of SEQ ID NO: 1 (C15), 1177-1195 of SEQ ID NO: 1 (C24), or 1275-1296 of SEQ ID NO: 1 (C31), or a conservative variant thereof.

In yet another aspect of this embodiment, an antibody composition selectively binds to an eptitope contained within a BoNT/A peptide having a length of at most 60 amino acids and consisting of at least 5 contiguous amino acids selected from one of the following BoNT/A amino acid sequences: 463-481 of SEQ ID NO:1 (N2), 505-523 of SEQ ID NO:1 (N5), 519-537 of SEQ ID NO:1 (N6), 533-551 of SEQ ID NO:1 (N7), 603-621 of SEQ ID NO:1 (N12), 645-663 of SEQ ID NO:1 (N15), 659-677 of SEQ ID NO:1 (N16), 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1079-1097 of SEQ ID NO:l (C17), 1107-1125 of SEQ ID NO:1 (C19), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28) or 1275-1296 of SEQ ID NO:1 (C31), or a conservative variant thereof. In yet another aspect of this embodiment, such a BoNT/A peptide is selected from one of the following amino acid sequences: 533-551 of SEQ ID NO:1 (N7), 659-677 of SEQ ID NO:1 (N16), 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1107-1125 of SEQ ID NO:1 (C19), 1163-1181 of SEQ ID NO:1 (C23), 1191-1209 of SEQ ID NO:1 (C25), 1233-1251 of SEQ ID NO:1 (C28) or 1275-1296 of SEQ ID NO:1 (C31), or a conservative variant thereof. In yet another aspect of this embodiment, such a BoNT/A peptide is selected from one of the following amino acid sequences: 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1079-1097 of SEQ ID NO:1 (C17), 1107-1125 of SEQ ID NO:1 (C19), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28) or 1275-1296 of SEQ ID NO:1 (C31), or a conservative variant thereof. In yet another aspect of this embodiment, such a BoNT/A peptide is selected from one of the following amino acid sequences: 729-747 of SEQ ID NO:1 (N21) 799-817 of SEQ ID NO:1 (N26), 1065-1083 of SEQ ID NO:1 (C16), 1163-1181 of SEQ ID NO:1 (C23) or 1275-1296 of SEQ ID NO:1 (C31), or a conservative variant thereof.

In yet another embodiment of the present invention, an antibody composition selectively binds to an eptitope contained within a BoNT/A peptide having a length of at most 60 amino acids and consisting of at least 5 contiguous amino acids selected from one of the following BoNT/A amino acid sequences: 449-467 of SEQ ID NO:1(N1), 463-481 of SEQ ID NO:1 (N2), 491-509 of SEQ ID NO:1 (N4), 505-523 of SEQ ID NO:1 (N5), 519-537 of SEQ ID NO:1 (N6), 533-551 of SEQ ID NO:1 (N7), 547-565 of SEQ ID NO:1 (N8), 561-579 of SEQ ID NO:1 (N9), 575-593 of SEQ ID NO:1 (N10), 589-607 of SEQ ID NO:1 (N11), 631-649 of SEQ ID NO:1 (N14), 645-663 of SEQ ID NO:1 (N15), 659-677 of SEQ ID NO:1 (N16), 673-691 of SEQ ID NO:1 (N17), 701-719 of SEQ ID NO:1 (N19), 715-733 of SEQ ID NO:1 (N20), 729-747 of SEQ ID NO:1 (N21), 743-761 of SEQ ID NO:1 (N22), 757-775 of SEQ ID NO:1 (N23), 771-789 of SEQ ID NO:1 (N24), 785-803 of SEQ ID NO:1 (N25), 799-817 of SEQ ID NO:1 (N26), 813-831 of SEQ ID NO:1 (N27), 827-845 of SEQ ID NO:1 (N28), 869-887 of SEQ ID NO:1 (C2), 883-901 of SEQ ID NO:1 (C3), 911-929 of SEQ ID NO:1 (C5), 925-943 of SEQ ID NO:1 (C6), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1065-1083 of SEQ ID NO:1 (C16), 1079-1097 of SEQ ID NO:1 (C17), 1107-1125 of SEQ ID NO:1 (C19), 1121-1139 of SEQ ID NO:1 (C20), 1135-1153 of SEQ ID NO:1 (C21), 1149-1167 of SEQ ID NO:1 (C22), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28), 1247-1265 of SEQ ID NO:1 (C29), 1261-1279 of SEQ ID NO:1 (C30), or 1275-1296 of SEQ ID NO:1 (C31), or a non-conservative variant thereof. In an aspect of this embodiment, such a BoNT/A peptide is selected from one of the following amino acid sequences: 463-481 of SEQ ID NO:1 (N2), 519-537 of SEQ ID NO:1 (N6), 533-551 of SEQ ID NO:1 (N7), 547-565 of SEQ ID NO:1 (N8), 561-579 of SEQ ID NO:1 (N9), 589-607 of SEQ ID NO:1 (N11), 659-677 of SEQ ID NO:1 (N16), 743-761 of SEQ ID NO:1 (N22), 785-803 of SEQ ID NO:1 (N25), 813-831 of SEQ ID NO:1 (N27), 827-845 of SEQ ID NO:1 (N28), 869-887 of SEQ ID NO:1 (C2), 883-901 of SEQ ID NO:1 (C3), 911-929 of SEQ ID NO:1 (C6), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1121-1139 of SEQ ID NO:1 (C20), 1135-1153 of SEQ ID NO:1 (C21), 1177-1195 of SEQ ID NO:1 (C24), 1261-1279 of SEQ ID NO:1 (C30) or 1275-1296 of SEQ ID NO:1 (C31), or a non-conservative variant thereof. In another aspect of this embodiment, such a BoNT/A peptide is selected from one of the following amino acid sequences: 463-481 of SEQ ID NO:1 (N2), 533-551 of SEQ ID NO:1 (N7), 547-565 of SEQ ID NO:1 (N8), 561-579 of SEQ ID NO:1 (N9), 659-677 of SEQ ID NO:1 (N16), 743-761 of SEQ ID NO:1 (N22), 785-803 of SEQ ID NO:1 (N25), 813-831 of SEQ ID NO:1 (N27), 883-901 of SEQ ID NO:1 (C3), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1121-1139 of SEQ ID NO:1 (C20) or 1177-1195 of SEQ ID NO:1 (C24) or 1275-1296 of SEQ ID NO: 1 (C31), or a non-conservative variant thereof. In another aspect of this embodiment, such a BoNT/A peptide is selected from one of the following amino acid sequences: 533-551 of SEQ ID NO: 1 (N7), 547-565 of SEQ ID NO: 1 (N8), 743-761 of SEQ ID NO: 1 (N22), 785-803 of SEQ ID NO: 1 (N25), 813-831 of SEQ ID NO: 1 (N27), 995-1013 of SEQ ID NO: 1 (C11); 1051-1069 of SEQ ID NO: 1 (C15), 1177-1195 of SEQ ID NO: 1 (C24), or 1275-1296 of SEQ ID NO: 1 (C31), or a non-conservative variant thereof.

In yet another aspect of this embodiment, an antibody composition selectively binds to an eptitope contained within a BoNT/A peptide having a length of at most 60 amino acids and consisting of at least 5 contiguous amino acids selected from one of the following BoNT/A amino acid sequences: 463-481 of SEQ ID NO:1 (N2), 505-523 of SEQ ID NO:1 (N5), 519-537 of SEQ ID NO:1 (N6), 533-551 of SEQ ID NO:1 (N7), 603-621 of SEQ ID NO:1 (N12), 645-663 of SEQ ID NO:1 (N15), 659-677 of SEQ ID NO:1 (N16), 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1079-1097 of SEQ ID NO:1 (C17), 1107-1125 of SEQ ID NO:1 (C19), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28) or 1275-1296 of SEQ ID NO:1 (C31), or a non-conservative variant thereof. In yet another aspect of this embodiment, such a BoNT/A peptide is selected from one of the following amino acid sequences: 533-551 of SEQ ID NO:1 (N7), 659-677 of SEQ ID NO:1 (N16), 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1107-1125 of SEQ ID NO:1 (C19), 1163-1181 of SEQ ID NO:1 (C23), 1191-1209 of SEQ ID NO:1 (C25), 1233-1251 of SEQ ID NO:1 (C28) or 1275-1296 of SEQ ID NO:1 (C31), or a non-conservative variant thereof. In yet another aspect of this embodiment, such a BoNT/A peptide is selected from one of the following amino acid sequences: 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1079-1097 of SEQ ID NO:1 (C17), 1107-1125 of SEQ ID NO:1 (C19), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28) or 1275-1296 of SEQ ID NO:1 (C31), or a non-conservative variant thereof. In yet another aspect of this embodiment, such a BoNT/A peptide is selected from one of the following amino acid sequences: 729-747 of SEQ ID NO:1 (N21) 799-817 of SEQ ID NO:1 (N26), 1065-1083 of SEQ ID NO:1 (C16), 1163-1181 of SEQ ID NO:1 (C23) or 1275-1296 of SEQ ID NO:1 (C31), or a non-conservative variant thereof.

In yet another embodiment of the present invention, an antibody composition selectively binds to an eptitope contained within a BoNT/A peptide having a length of at most 60 amino acids and consisting of at least 5 contiguous amino acids selected from one of the following BoNT/A amino acid sequences: 449-467 of SEQ ID NO:1(N1), 463-481 of SEQ ID NO:1 (N2), 491-509 of SEQ ID NO:1 (N4), 505-523 of SEQ ID NO:1 (N5), 519-537 of SEQ ID NO:1 (N6), 533-551 of SEQ ID NO:1 (N7), 547-565 of SEQ ID NO:1 (N8), 561-579 of SEQ ID NO:1 (N9), 575-593 of SEQ ID NO:1 (N10), 589-607 of SEQ ID NO:1 (N11), 631-649 of SEQ ID NO:1 (N14), 645-663 of SEQ ID NO:1 (N15), 659-677 of SEQ ID NO:1 (N16), 673-691 of SEQ ID NO:1 (N17), 701-719 of SEQ ID NO:1 (N19), 715-733 of SEQ ID NO:1 (N20), 729-747 of SEQ ID NO:1 (N21), 743-761 of SEQ ID NO:1 (N22), 757-775 of SEQ ID NO:1 (N23), 771-789 of SEQ ID NO:1 (N24), 785-803 of SEQ ID NO:1 (N25), 799-817 of SEQ ID NO:1 (N26), 813-831 of SEQ ID NO:1 (N27), 827-845 of SEQ ID NO:1 (N28), 869-887 of SEQ ID NO:1 (C2), 883-901 of SEQ ID NO:1 (C3), 911-929 of SEQ ID NO:1 (C5), 925-943 of SEQ ID NO:1 (C6), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1065-1083 of SEQ ID NO:1 (C16), 1079-1097 of SEQ ID NO:1 (C17), 1107-1125 of SEQ ID NO:1 (C19), 1121-1139 of SEQ ID NO:1 (C20), 1135-1153 of SEQ ID NO:1 (C21), 1149-1167 of SEQ ID NO:1 (C22), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28), 1247-1265 of SEQ ID NO:1 (C29), 1261-1279 of SEQ ID NO:1 (C30), or 1275-1296 of SEQ ID NO:1 (C31), or an immunoreactive fragment thereof, with the proviso that the BoNT/A peptide is not SEQ ID NO:2. In an aspect of this embodiment, such a BoNT/A peptide is selected from one of the following amino acid sequences: 463-481 of SEQ ID NO:1 (N2), 519-537 of SEQ ID NO:1 (N6), 533-551 of SEQ ID NO:1 (N7), 547-565 of SEQ ID NO:1 (N8), 561-579 of SEQ ID NO:1 (N9), 589-607 of SEQ ID NO:1 (N11), 659-677 of SEQ ID NO:1 (N16), 743-761 of SEQ ID NO:1 (N22), 785-803 of SEQ ID NO:1 (N25), 813-831 of SEQ ID NO:1 (N27), 827-845 of SEQ ID NO:1 (N28), 869-887 of SEQ ID NO:1 (C2), 883-901 of SEQ ID NO:1 (C3), 911-929 of SEQ ID NO:1 (C6), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1121-1139 of SEQ ID NO:1 (C20), 1135-1153 of SEQ ID NO:1 (C21), 1177-1195 of SEQ ID NO:1 (C24), 1261-1279 of SEQ ID NO:1 (C30) or 1275-1296 of SEQ ID NO:1 (C31), or an immunoreactive fragment thereof, with the proviso that the BoNT/A peptide is not SEQ ID NO:2. In another aspect of this embodiment, such a BoNT/A peptide is selected from one of the following amino acid sequences: 463-481 of SEQ ID NO:1 (N2), 533-551 of SEQ ID NO:1 (N7), 547-565 of SEQ ID NO:1 (N8), 561-579 of SEQ ID NO:1 (N9), 659-677 of SEQ ID NO:1 (N16), 743-761 of SEQ ID NO:1 (N22), 785-803 of SEQ ID NO:1 (N25), 813-831 of SEQ ID NO:1 (N27), 883-901 of SEQ ID NO:1 (C3), 939-957 of SEQ ID NO:1 (C7), 967-985 of SEQ ID NO:1 (C9), 981-999 of SEQ ID NO:1(C10), 995-1013 of SEQ ID NO:1 (C11), 1051-1069 of SEQ ID NO:1 (C15), 1121-1139 of SEQ ID NO:1 (C20) or 1177-1195 of SEQ ID NO:1 (C24) or 1275-1296 of SEQ ID NO: 1 (C31), or an immunoreactive fragment thereof, with the proviso that the BoNT/A peptide is not SEQ ID NO:2. In another aspect of this embodiment, such a BoNT/A peptide is selected from one of the following amino acid sequences: 533-551 of SEQ ID NO: 1 (N7), 547-565 of SEQ ID NO: 1 (N8), 743-761 of SEQ ID NO: 1 (N22), 785-803 of SEQ ID NO: 1 (N25), 813-831 of SEQ ID NO: 1 (N27), 995-1013 of SEQ ID NO: 1 (C11); 1051-1069 of SEQ ID NO: 1 (C15), 1177-1195 of SEQ ID NO: 1 (C24), or 1275-1296 of SEQ ID NO: 1 (C31), or an immunoreactive fragment thereof, with the proviso that the BoNT/A peptide is not SEQ ID NO:2.

In yet another aspect of this embodiment, an antibody composition selectively binds to an eptitope contained within a BoNT/A peptide having a length of at most 60 amino acids and consisting of at least 5 contiguous amino acids selected from one of the following BoNT/A amino acid sequences: 463-481 of SEQ ID NO:1 (N2), 505-523 of SEQ ID NO:1 (N5), 519-537 of SEQ ID NO:1 (N6), 533-551 of SEQ ID NO:1 (N7), 603-621 of SEQ ID NO:1 (N12), 645-663 of SEQ ID NO:1 (N15), 659-677 of SEQ ID NO:1 (N16), 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1079-1097 of SEQ ID NO:1 (C17), 1107-1125 of SEQ ID NO:1 (C19), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28) or 1275-1296 of SEQ ID NO:1 (C31), or an immunoreactive fragment thereof, with the proviso that the BoNT/A peptide is not SEQ ID NO:2. In yet another aspect of this embodiment, such a BoNT/A peptide is selected from one of the following amino acid sequences: 533-551 of SEQ ID NO:1 (N7), 659-677 of SEQ ID NO:1 (N16), 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1107-1125 of SEQ ID NO:1 (C19), 1163-1181 of SEQ ID NO:1 (C23), 1191-1209 of SEQ ID NO:1 (C25), 1233-1251 of SEQ ID NO:1 (C28) or 1275-1296 of SEQ ID NO:1 (C31), or an immunoreactive fragment thereof, with the proviso that the BoNT/A peptide is not SEQ ID NO:2. In yet another aspect of this embodiment, such a BoNT/A peptide is selected from one of the following amino acid sequences: 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1079-1097 of SEQ ID NO:1 (C17), 1107-1125 of SEQ ID NO:1 (C19), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28) or 1275-1296 of SEQ ID NO:1 (C31), or an immunoreactive fragment thereof, with the proviso that the BoNT/A peptide is not SEQ ID NO:2. In yet another aspect of this embodiment, such a BoNT/A peptide is selected from one of the following amino acid sequences: 729-747 of SEQ ID NO:1 (N21) 799-817 of SEQ ID NO:1 (N26), 1065-1083 of SEQ ID NO:1 (C16), 1163-1181 of SEQ ID NO:1 (C23) or 1275-1296 of SEQ ID NO:1 (C31), or an immunoreactive fragment thereof, with the proviso that the BoNT/A peptide is not SEQ ID NO:2.

In is envisioned that a BoNT/A peptide useful as an epitope for an antibody composition disclosed in the present specification can have any of a variety of lengths from at least 5 amino acids to at most 60 amino acids. Therefore, aspects of this embodiment may include a BoNT/A peptide with at least, e.g., five amino acids, six amino acids, seven amino acids, eight amino acids, nine amino acids, ten amino acids, 11 amino acids, 12 amino acids, 13 amino acids, 14 amino acids, 15 amino acids, 16 amino acids, 17 amino acids, 18 amino acids, 19 amino acids, 20 amino acids, 25 amino acids, 30 amino acids, 35 amino acids, 40 amino acids, 45 amino acids, 50 amino acids, 55 amino acids or 60 amino acids. Other aspects of this embodiment may include a BoNT/A peptide with at least, e.g., five amino acids of SEQ ID NO:1, six amino acids of SEQ ID NO:1, seven amino acids of SEQ ID NO:1, eight amino acids of SEQ ID NO:1, nine amino acids of SEQ ID NO:1, ten amino acids of SEQ ID NO:1, 11 amino acids of SEQ ID NO:1, 12 amino acids of SEQ ID NO:1, 13 amino acids of SEQ ID NO:1, 14 amino acids of SEQ ID NO:1, 15 amino acids of SEQ ID NO:1, 16 amino acids of SEQ ID NO:1, 17 amino acids of SEQ ID NO:1, 18 amino acids of SEQ ID NO:1 , 19 amino acids of SEQ ID NO:1, 20 amino acids of SEQ ID NO:1, 25 amino acids of SEQ ID NO:1, 30 amino acids of SEQ ID NO:1, 35 amino acids of SEQ ID NO:1, 40 amino acids of SEQ ID NO:1, 45 amino acids of SEQ ID NO:1, 50 amino acids of SEQ ID NO:1, 55 amino acids or 60 amino acids of SEQ ID NO:1. In further embodiments, such a BoNT/A peptide of the invention may include a BoNT/A peptide with at least, e.g., five amino acids, six amino acids, seven amino acids, eight amino acids, nine amino acids, ten amino acids, 11 amino acids, 12 amino acids, 13 amino acids, 14 amino acids, 15 amino acids, 16 amino acids, 17 amino acids, 18 amino acids, 19 amino acids, 20 amino acids, 25 amino acids, 30 amino acids, 35 amino acids, 40 amino acids, 45 amino acids, 50 amino acids, 55 amino acids or 60 amino acids and consist of at least 5 contiguous amino acids selected from one of the following BoNT/A amino acid sequences: 449-467 of SEQ ID NO:1 (N1), 463-481 of SEQ ID NO:1 (N2), 491-509 of SEQ ID NO:1 (N4), 505-523 of SEQ ID NO:1 (N5), 519-537 of SEQ ID NO:1 (N6), 533-551 of SEQ ID NO:1 (N7), 547-565 of SEQ ID NO:1 (N8), 561-579 of SEQ ID NO:1 (N9), 575-593 of SEQ ID NO:1 (N10), 589-607 of SEQ ID NO:1 (N11), 631-649 of SEQ ID NO:1 (N14), 645-663 of SEQ ID NO:1 (N15), 659-677 of SEQ ID NO:1 (N16), 673-691 of SEQ ID NO:1 (N17), 701-719 of SEQ ID NO:1 (N19), 715-733 of SEQ ID NO:1 (N20), 729-747 of SEQ ID NO:1 (N21), 743-761 of SEQ ID NO:1 (N22), 757-775 of SEQ ID NO:1 (N23), 771-789 of SEQ ID NO:1 (N24), 785-803 of SEQ ID NO:1 (N25), 799-817 of SEQ ID NO:1 (N26), 813-831 of SEQ ID NO:1 (N27), 827-845 of SEQ ID NO:1 (N28), 869-887 of SEQ ID NO:1 (C2), 883-901 of SEQ ID NO:1 (C3), 911-929 of SEQ ID NO:1 (C5), 925-943 of SEQ ID NO:1 (C6), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1065-1083 of SEQ ID NO:1 (C16), 1079-1097 of SEQ ID NO:1 (C17), 1107-1125 of SEQ ID NO:1 (C19), 1121-1139 of SEQ ID NO:1 (C20), 1135-1153 of SEQ ID NO:1 (C21), 1149-1167 of SEQ ID NO:1 (C22), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28), 1247-1265 of SEQ ID NO:1 (C29), 1261-1279 of SEQ ID NO:1 (C30) or 1275-1296 of SEQ ID NO:1 (C31), or a conservative variant, a non-conservative variant, or immunoreactive fragment thereof, with the proviso that the BoNT/A peptide is not SEQ ID NO:2.

In another embodiment of the present invention, a BoNT/A peptide useful as an epitope for an antibody composition can comprise one BoNT/A peptide disclosed in the present specification. In another embodiment of the present invention, a BoNT/A peptide useful as an epitope for an antibody composition can comprise a plurality of BoNT/A peptides disclosed in the present specification. Thus, aspects of this embodiment can include one or more BoNT/A peptides, two or more BoNT/A peptides, three or more BoNT/A peptides, four or more BoNT/A peptides, five or more BoNT/A peptides, six or more BoNT/A peptides, seven or more BoNT/A peptides, eight or more BoNT/A peptides, nine or more BoNT/A peptides, ten or more BoNT/A peptides, 15 or more BoNT/A peptides, 20 or more BoNT/A peptides, 25 or more BoNT/A peptides or 30 or more BoNT/A peptides. In other aspects of this embodiment can include one or more BoNT/A conservative variants, two or more BoNT/A conservative variants, three or more BoNT/A conservative variants, four or more BoNT/A conservative variants, five or more BoNT/A conservative variants, six or more BoNT/A conservative variants, seven or more BoNT/A conservative variants, eight or more BoNT/A conservative variants, nine or more BoNT/A conservative variants, ten or more BoNT/A conservative variants, 15 or more BoNT/A conservative variants, 20 or more BoNT/A conservative variants, 25 or more BoNT/A conservative variants or 30 or more BoNT/A conservative variants. In further aspects of this embodiment can include one or more BoNT/A non-conservative variants, two or more BoNT/A non-conservative variants, three or more BoNT/A non-conservative variants, four or more BoNT/A non-conservative variants, five or more BoNT/A non-conservative variants, six or more BoNT/A non-conservative variants, seven or more BoNT/A non-conservative variants, eight or more BoNT/A non-conservative variants, nine or more BoNT/A non-conservative variants, ten or more BoNT/A non-conservative variants, 15 or more BoNT/A non-conservative variants, 20 or more BoNT/A non-conservative variants, 25 or more BoNT/A non-conservative variants or 30 or more BoNT/A non-conservative variants. In still other aspects of this embodiment can include one or more BoNT/A immunoreactive fragments, two or more BoNT/A immunoreactive fragments, three or more BoNT/A immunoreactive fragments, four or more BoNT/A immunoreactive fragments, five or more BoNT/A immunoreactive fragments, six or more BoNT/A immunoreactive fragments, seven or more BoNT/A immunoreactive fragments, eight or more BoNT/A immunoreactive fragments, nine or more BoNT/A peptides, ten or more BoNT/A immunoreactive fragments, 15 or more BoNT/A immunoreactive fragments, 20 or more BoNT/A immunoreactive fragments, 25 or more BoNT/A immunoreactive fragments or 30 or more BoNT/A immunoreactive fragments. BoNT/A peptides disclosed in the present specification useful as an epitope for an antibody composition can be selected, for example, depending on immunological factors, such as potency of the peptide in inducing an immune response, and technical factors, such as chemical synthesis yields. It is also understood that the two or more BoNT/A peptides can be provided separately or as part of a compound molecule such as a chimeric peptide or heterologous protein.

It is also envisioned that any and all combinations of BoNT/A peptides disclosed in the specification, including, e.g., BoNT/A peptides of SEQ ID NO:1, BoNT/A conservative variants, BoNT/A non-conservative variants and BoNT/A immunoreactive fragments, can be useful as an epitope for an antibody composition. Thus, aspects of this embodiment include one or more BoNT/A peptides comprising one or more BoNT/A peptides of SEQ ID NO: 1 and one or more BoNT/A conservative variants; one or more BoNT/A peptides of SEQ ID NO: 1 and one or more BoNT/A non-conservative variants; one or more BoNT/A peptides of SEQ ID NO: 1 and one or more BoNT/A immunoreactive fragments; one or more BoNT/A conservative variants and one or more BoNT/A non-conservative variants; one or more BoNT/A conservative variants and one or more BoNT/A immunoreactive fragments; one or more BoNT/A non-conservative variants and one or more BoNT/A immunoreactive fragments; one or more BoNT/A peptides of SEQ ID NO: 1, one or more BoNT/A conservative variants and one or more BoNT/A non-conservative variants; one or more BoNT/A peptides of SEQ ID NO: 1, one or more BoNT/A conservative variants and one or more BoNT/A immunoreactive fragments; one or more BoNT/A peptides of SEQ ID NO: 1, one or more BoNT/A non-conservative variants and one or more BoNT/A immunoreactive fragments; one or more BoNT/A conservative variants, one or more BoNT/A non-conservative variants and one or more BoNT/A immunoreactive fragments; or one or more BoNT/A peptides of SEQ ID NO: 1, one or more BoNT/A conservative variants, one or more BoNT/A non-conservative variants and one or more BoNT/A immunoreactive fragments.

The term “antibody”, as used herein, includes polyclonal and monoclonal antibodies, as well as antigenic compound-binding fragments of such antibodies including, without limitation, Fab, F(ab′).sub.2, Fd, Fv fragments, and single chain derivatives of the same. “Antibody” also includes cell-associated antibodies, such as Ig receptors, for example. In addition, the term “antibody” includes naturally occurring antibodies, as well as non-naturally occurring antibodies, including, for example, chimeric, bifunctional, and humanized antibodies, and related synthetic isoforms. As used herein, an “epitope” means the site on an antigen that is recognized and bound by a particular antibody or T-cell receptor. The minimal size of a protein epitope, as defined herein, is about five amino acids, and a protein epitope typically comprises at least eight amino acids. It is to be noted, however, that an epitope might comprise a portion of an antigen other than the amino acid sequence, e.g., a carbohydrate moiety or a lipid moiety. Furthermore, an epitope may be discontinuous, i.e., it comprises amino acid residues that are not adjacent in the polypeptide but are brought together into an epitope by way of the secondary, tertiary, or quaternary structure of the protein. As used herein, the term “selectively binds” means the discriminatory binding of the antibody to the indicated target peptide or polypeptide such that the antibody does not substantially cross react with unrelated peptides or polypeptides. Specific reactivity can include binding properties such as binding specificity, binding affinity and binding avidity. For example, an antibody can bind a target peptide or polypeptide with a binding affinity (Kd) of about 10⁻⁴ M or more, 10⁻⁶ M or more, 10⁻⁷ M or more, 10⁻⁸ M or more, 10⁻⁹ M or more, or 10⁻¹⁰ M or more. Specific protocols for making and using antibodies as well as detecting, and measuring antibody binding are known in the art and disclosed herein, see, e.g., Harlow & Lane, supra, 1998a; Harlow & Lane, supra, 1998b; MOLECULAR CLONING, A LABORATORY MANUAL, supra, 2001; and CURRENT PROTOCOLS IN MOLECULAR BIOLOGY, supra, 2004. BoNT/A peptides disclosed in the present specification used to selectively bind an antibody composition disclosed in the present specification can be selected, for example, depending on immunological factors, such as potency of the peptide in inducing a tolerogizing response, and technical factors, such as chemical synthesis yields.

VI. Methods of Determining BoNT/A Immunoresistance

As described above, patients treated with botulinum toxin can develop immunoresistance to the therapeutic treatment, reducing or eliminating the beneficial effect of botulinum toxin therapy. Methods that determine whether a patient is mounting an immune response against a BoNT/A peptide are of major importance. These assays would allow the immunoresponsive state of the patient to be evaluated periodically during the course of a BoNT/A therapy. By knowing the predisposition of an individual 1) the potential value of a specific BoNT/A treatment can be determined prior to its administration to a patient; and 2) the possible benefit from continued BoNT/A therapy can be assessed and any possible adjustments to a treatment determined. Therefore, these assays present a major benefit in terms of providing better patient care and reducing health care costs. The BoNT/A peptides disclosed in the present specification are useful in methods of determining immunoresistance to botulinum toxin therapy in an individual. These peptides each contain one or more epitopes recognized by antibodies contained in antisera from animals immunized with BoNT/A, and thus can serve as binding substrates for anti-BoNT/A antibodies.

Thus, the present invention provides a method of determining immunoresistance to botulinum toxin therapy in a human or other mammal by determining the presence or absence in the human or other mammal of antibodies immunoreactive with a BoNT/A peptide composition disclosed in the present specification, where the presence of antibodies immunoreactive with the a BoNT/A peptide indicates immunoresistance to BoNT/A therapy. In one embodiment of the present invention, a method of determining the the presence or absence of an anti-BoNT/A antibody in a human or other mammal comprises the steps of combining a BoNT/A peptide and test sample and detecting the amount of complexes formed by said BoNT/A peptide and anti-BoNT/A antibody. In a preferred embodiment of the present invention, a method of determining the the presence or absence of an anti-BoNT/A antibody in a human or other mammal comprises the steps of combining a BoNT/A peptide and a test sample, detecting the amount of complexes formed by said BoNT/A peptide and BoNT/A antibody and correlating the amount of said complexes formed from said test sample relative to the amount of complexes formed by said BoNT/A peptide and said antibody from a control sample.

In one aspect of the present invention, all steps of a method for determining the presence or absence of a BoNT/A antibody are performed in solution. In other aspects of the method disclosed in the present ispecification, it is also envisioned that a method can optionally attach an assay component to a solid or insoluble material. Such a solid support can be, without limitation, e.g., a tube; plate; pins or “dipsticks”, column; particle, bead or other spherical or fibrous chromatographic media, such as, e.g., agarose beads, sepharose beads, silica beads and plastic beads; sheets or membranes, such as, e.g., nitrocellulose and polyvinylidene fluoride (PVDF). The solid support selected can have a physical property that renders it readily separable from soluble or unbound material and generally allows unbound materials, such as, e.g., excess reagents, reaction by-products, or solvents, to be separated or otherwise removed (by, e.g., washing, filtration, centrifugation, etc.) from solid support-bound assay component. Non-limiting examples of how to make and use a solid support-bound assay component are described in, e.g., MOLECULAR CLONING, A LABORATORY MANUAL, supra, 2001; and CURRENT PROTOCOLS IN MOLECULAR BIOLOGY, supra, 2004.

In an embodiment of the present invention, a BoNT/A peptide useful in a method disclosed in the present specification for determining the presence or absence of an anti-BoNT/A antibody has a length of at most 60 amino acids and consisting of at least 5 contiguous amino acids selected from one of the following BoNT/A amino acid sequences: 449-467 of SEQ ID NO:1 (N1), 463-481 of SEQ ID NO:1 (N2), 491-509 of SEQ ID NO:1 (N4), 505-523 of SEQ ID NO:1 (N5), 519-537 of SEQ ID NO:1 (N6), 533-551 of SEQ ID NO:1 (N7), 547-565 of SEQ ID NO:1 (N8), 561-579 of SEQ ID NO:1 (N9), 575-593 of SEQ ID NO:1 (N10), 589-607 of SEQ ID NO:1 (N11), 631-649 of SEQ ID NO:1 (N14), 645-663 of SEQ ID NO:1 (N15), 659-677 of SEQ ID NO:1 (N16), 673-691 of SEQ ID NO:1 (N17), 701-719 of SEQ ID NO:1 (N19), 715-733 of SEQ ID NO:1 (N20), 729-747 of SEQ ID NO:1 (N21), 743-761 of SEQ ID NO:1 (N22), 757-775 of SEQ ID NO:1 (N23), 771-789 of SEQ ID NO:1 (N24), 785-803 of SEQ ID NO:1 (N25), 799-817 of SEQ ID NO:1 (N26), 813-831 of SEQ ID NO:1 (N27), 827-845 of SEQ ID NO:1 (N28), 869-887 of SEQ ID NO:1 (C2), 883-901 of SEQ ID NO:1 (C3), 911-929 of SEQ ID NO:1 (C5), 925-943 of SEQ ID NO:1 (C6), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1065-1083 of SEQ ID NO:1 (C16), 1079-1097 of SEQ ID NO:1 (C17), 1107-1125 of SEQ ID NO:1 (C19), 1121-1139 of SEQ ID NO:1 (C20), 1135-1153 of SEQ ID NO:1 (C21), 1149-1167 of SEQ ID NO:1 (C22), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28), 1247-1265 of SEQ ID NO:1 (C29), 1261-1279 of SEQ ID NO:1 (C30), or 1275-1296 of SEQ ID NO:1 (C31), or a conservative variant or immunoreactive fragment thereof, with the proviso that the BoNT/A peptide is not SEQ ID NO:2.

In another embodiment of the present invention, a BoNT/A peptide useful in a method disclosed in the present specification for determining the presence or absence of an anti-BoNT/A antibody has a length of at most 60 amino acids and consisting of at least 5 contiguous amino acids selected from one of the following BoNT/A amino acid sequences: 449-467 of SEQ ID NO:1(N1), 463-481 of SEQ ID NO:1 (N2), 491-509 of SEQ ID NO:1 (N4), 505-523 of SEQ ID NO:1 (N5), 519-537 of SEQ ID NO:1 (N6), 533-551 of SEQ ID NO:1 (N7), 547-565 of SEQ ID NO:1 (N8), 561-579 of SEQ ID NO:1 (N9), 575-593 of SEQ ID NO:1 (N10), 589-607 of SEQ ID NO:1 (N11), 631-649 of SEQ ID NO:1 (N14), 645-663 of SEQ ID NO:1 (N15), 659-677 of SEQ ID NO:1 (N16), 673-691 of SEQ ID NO:1 (N17), 701-719 of SEQ ID NO:1 (N19), 715-733 of SEQ ID NO:1 (N20), 729-747 of SEQ ID NO:1 (N21), 743-761 of SEQ ID NO:1 (N22), 757-775 of SEQ ID NO:1 (N23), 771-789 of SEQ ID NO:1 (N24), 785-803 of SEQ ID NO:1 (N25), 799-817 of SEQ ID NO:1 (N26), 813-831 of SEQ ID NO:1 (N27), 827-845 of SEQ ID NO:1 (N28), 869-887 of SEQ ID NO:1 (C2), 883-901 of SEQ ID NO:1 (C3), 911-929 of SEQ ID NO:1 (C5), 925-943 of SEQ ID NO:1 (C6), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1065-1083 of SEQ ID NO:1 (C16), 1079-1097 of SEQ ID NO:1 (C17), 1107-1125 of SEQ ID NO:1 (C19), 1121-1139 of SEQ ID NO:1 (C20), 1135-1153 of SEQ ID NO:1 (C21), 1149-1167 of SEQ ID NO:1 (C22), 1163-1181 of SEQ ID NO:1 (C23), 1177-1195 of SEQ ID NO:1 (C24),1191-1209 of SEQ ID NO:1 (C25), 1233-1251 of SEQ ID NO:1 (C28), 1247-1265 of SEQ ID NO:1 (C29), 1261-1279 of SEQ ID NO:1 (C30), or 1275-1296 of SEQ ID NO:1 (C31). In an aspect of this embodiment, such a BoNT/A peptide is selected from one of the following amino acid sequences: 463-481 of SEQ ID NO:1 (N2), 519-537 of SEQ ID NO:1 (N6), 533-551 of SEQ ID NO:1 (N7), 547-565 of SEQ ID NO:1 (N8), 561-579 of SEQ ID NO:1 (N9), 589-607 of SEQ ID NO:1 (N11), 659-677 of SEQ ID NO:1 (N16), 743-761 of SEQ ID NO:1 (N22), 785-803 of SEQ ID NO:1 (N25), 813-831 of SEQ ID NO:1 (N27), 827-845 of SEQ ID NO:1 (N28), 869-887 of SEQ ID NO:1 (C2), 883-901 of SEQ ID NO:1 (C3), 911-929 of SEQ ID NO:1 (C6), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1121-1139 of SEQ ID NO:1 (C20), 1135-1153 of SEQ ID NO:1 (C21), 1177-1195 of SEQ ID NO:1 (C24), 1261-1279 of SEQ ID NO:1 (C30) or 1275-1296 of SEQ ID NO:1 (C31). In another aspect of this embodiment, such a BoNT/A peptide is selected from one of the following amino acid sequences: 463-481 of SEQ ID NO:1 (N2), 533-551 of SEQ ID NO:1 (N7), 547-565 of SEQ ID NO:1 (N8), 561-579 of SEQ ID NO:1 (N9), 659-677 of SEQ ID NO:1 (N16), 743-761 of SEQ ID NO:1 (N22), 785-803 of SEQ ID NO:1 (N25), 813-831 of SEQ ID NO:1 (N27), 883-901 of SEQ ID NO:1 (C3), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1121-1139 of SEQ ID NO:1 (C20) or 1177-1195 of SEQ ID NO:1 (C24) or 1275-1296 of SEQ ID NO: 1 (C31). In another aspect of this embodiment, such a BoNT/A peptide is selected from one of the following amino acid sequences: 533-551 of SEQ ID NO: 1 (N7), 547-565 of SEQ ID NO: 1 (N8), 743-761 of SEQ ID NO: 1 (N22), 785-803 of SEQ ID NO: 1 (N25), 813-831 of SEQ ID NO: 1 (N27), 995-1013 of SEQ ID NO: 1 (C11); 1051-1069 of SEQ ID NO: 1 (C15), 1177-1195 of SEQ ID NO: 1 (C24), or 1275-1296 of SEQ ID NO: 1 (C31).

In yet another aspect of this embodiment, a BoNT/A peptide useful in a method disclosed in the present specification for determining the presence or absence of an anti-BoNT/A antibody has a length of at most 60 amino acids and consisting of at least 5 contiguous amino acids selected from one of the following BoNT/A amino acid sequences: 463-481 of SEQ ID NO:1 (N2), 505-523 of SEQ ID NO:1 (N5), 519-537 of SEQ ID NO:1 (N6), 533-551 of SEQ ID NO:1 (N7), 603-621 of SEQ ID NO:1 (N12), 645-663 of SEQ ID NO:1 (N15), 659-677 of SEQ ID NO:1 (N16), 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1079-1097 of SEQ ID NO:1 (C17), 1107-1125 of SEQ ID NO:1 (C19), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28) or 1275-1296 of SEQ ID NO:1 (C31). In yet another aspect of this embodiment, such a BoNT/A peptide is selected from one of the following amino acid sequences: 533-551 of SEQ ID NO:1 (N7), 659-677 of SEQ ID NO:1 (N16), 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1107-1125 of SEQ ID NO:1 (C19), 1163-1181 of SEQ ID NO:1 (C23), 1191-1209 of SEQ ID NO:1 (C25), 1233-1251 of SEQ ID NO:1 (C28) or 1275-1296 of SEQ ID NO:1 (C31). In yet another aspect of this embodiment, such a BoNT/A peptide is selected from one of the following amino acid sequences: 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1079-1097 of SEQ ID NO:1 (C17), 1107-1125 of SEQ ID NO:1 (C19), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28) or 1275-1296 of SEQ ID NO:1 (C31). In yet another aspect of this embodiment, such a BoNT/A peptide is selected from one of the following amino acid sequences: 729-747 of SEQ ID NO:1 (N21) 799-817 of SEQ ID NO:1 (N26), 1065-1083 of SEQ ID NO:1 (C16), 1163-1181 of SEQ ID NO:1 (C23) or 1275-1296 of SEQ ID NO:1 (C31).

In yet another embodiment of the present invention, a BoNT/A peptide useful in a method disclosed in the present specification for determining the presence or absence of an anti-BoNT/A antibody is selected from one of the following BoNT/A amino acid sequences: 449-467 of SEQ ID NO:1(N1), 463-481 of SEQ ID NO:1 (N2), 491-509 of SEQ ID NO:1 (N4), 505-523 of SEQ ID NO:1 (N5), 519-537 of SEQ ID NO:1 (N6), 533-551 of SEQ ID NO:1 (N7), 547-565 of SEQ ID NO:1 (N8), 561-579 of SEQ ID NO:1 (N9), 575-593 of SEQ ID NO:1 (N10), 589-607 of SEQ ID NO:1 (N11), 631-649 of SEQ ID NO:1 (N14), 645-663 of SEQ ID NO:1 (N15), 659-677 of SEQ ID NO:1 (N16), 673-691 of SEQ ID NO:1 (N17), 701-719 of SEQ ID NO:1 (N19), 715-733 of SEQ ID NO:1 (N20), 729-747 of SEQ ID NO:1 (N21), 743-761 of SEQ ID NO:1 (N22), 757-775 of SEQ ID NO:1 (N23), 771-789 of SEQ ID NO:1 (N24), 785-803 of SEQ ID NO:1 (N25), 799-817 of SEQ ID NO:1 (N26), 813-831 of SEQ ID NO:1 (N27), 827-845 of SEQ ID NO:1 (N28), 869-887 of SEQ ID NO:1 (C2), 883-901 of SEQ ID NO:1 (C3), 911-929 of SEQ ID NO:1 (C5), 925-943 of SEQ ID NO:1 (C6), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1065-1083 of SEQ ID NO:1 (C16), 1079-1097 of SEQ ID NO:1 (C17), 1107-1125 of SEQ ID NO:1 (C19), 1121-1139 of SEQ ID NO:1 (C20), 1135-1153 of SEQ ID NO:1 (C21), 1149-1167 of SEQ ID NO:1 (C22), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28), 1247-1265 of SEQ ID NO:1 (C29), 1261-1279 of SEQ ID NO:1 (C30), or 1275-1296 of SEQ ID NO:1 (C31). In an aspect of this embodiment, such a BoNT/A peptide is selected from one of the following amino acid sequences: 463-481 of SEQ ID NO:1 (N2), 519-537 of SEQ ID NO:1 (N6), 533-551 of SEQ ID NO:1 (N7), 547-565 of SEQ ID NO:1 (N8), 561-579 of SEQ ID NO:1 (N9), 589-607 of SEQ ID NO:1 (N11), 659-677 of SEQ ID NO:1 (N16), 743-761 of SEQ ID NO:1 (N22), 785-803 of SEQ ID NO:1 (N25), 813-831 of SEQ ID NO:1 (N27), 827-845 of SEQ ID NO:1 (N28), 869-887 of SEQ ID NO:1 (C2), 883-901 of SEQ ID NO:1 (C3), 911-929 of SEQ ID NO:1 (C6), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1121-1139 of SEQ ID NO:1 (C20), 1135-1153 of SEQ ID NO:1 (C21), 1177-1195 of SEQ ID NO:1 (C24), 1261-1279 of SEQ ID NO:1 (C30) or 1275-1296 of SEQ ID NO:1 (C31). In another aspect of this embodiment, such a BoNT/A peptide is selected from one of the following amino acid sequences: 463-481 of SEQ ID NO:1 (N2), 533-551 of SEQ ID NO:1 (N7), 547-565 of SEQ ID NO:1 (N8), 561-579 of SEQ ID NO:1 (N9), 659-677 of SEQ ID NO:1 (N16), 743-761 of SEQ ID NO:1 (N22), 785-803 of SEQ ID NO:1 (N25), 813-831 of SEQ ID NO:1 (N27), 883-901 of SEQ ID NO:1 (C3), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1121-1139 of SEQ ID NO:1 (C20) or 1177-1195 of SEQ ID NO:1 (C24) or 1275-1296 of SEQ ID NO: 1 (C31). In another aspect of this embodiment, such a BoNT/A peptide is selected from one of the following amino acid sequences: 533-551 of SEQ ID NO: 1 (N7), 547-565 of SEQ ID NO: 1 (N8), 743-761 of SEQ ID NO: 1 (N22), 785-803 of SEQ ID NO: 1 (N25), 813-831 of SEQ ID NO: 1 (N27), 995-1013 of SEQ ID NO: 1 (C11); 1051-1069 of SEQ ID NO: 1 (C15), 1177-1195 of SEQ ID NO: 1 (C24), or 1275-1296 of SEQ ID NO: 1 (C31).

In yet another aspect of this embodiment, a BoNT/A peptide useful in a method disclosed in the present specification for determining the presence or absence of an anti-BoNT/A antibody is selected from one of the following BoNT/A amino acid sequences: 463-481 of SEQ ID NO:1 (N2), 505-523 of SEQ ID NO:1 (N5), 519-537 of SEQ ID NO:1 (N6), 533-551 of SEQ ID NO:1 (N7), 603-621 of SEQ ID NO:1 (N12), 645-663 of SEQ ID NO:1 (N15), 659-677 of SEQ ID NO:1 (N16), 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1079-1097 of SEQ ID NO:1 (C17), 1107-1125 of SEQ ID NO:1 (C19), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28) or 1275-1296 of SEQ ID NO:1 (C31). In yet another aspect of this embodiment, such a BoNT/A peptide is selected from one of the following amino acid sequences: 533-551 of SEQ ID NO:1 (N7), 659-677 of SEQ ID NO:1 (N16), 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1107-1125 of SEQ ID NO:1 (C19), 1163-1181 of SEQ ID NO:1 (C23), 1191-1209 of SEQ ID NO:1 (C25), 1233-1251 of SEQ ID NO:1 (C28) or 1275-1296 of SEQ ID NO:1 (C31). In yet another aspect of this embodiment, such a BoNT/A peptide is selected from one of the following amino acid sequences: 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1079-1097 of SEQ ID NO:1 (C17), 1107-1125 of SEQ ID NO:1 (C19), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28) or 1275-1296 of SEQ ID NO:1 (C31). In yet another aspect of this embodiment, such a BoNT/A peptide is selected from one of the following amino acid sequences: 729-747 of SEQ ID NO:1 (N21) 799-817 of SEQ ID NO:1 (N26), 1065-1083 of SEQ ID NO:1 (C16), 1163-1181 of SEQ ID NO:1 (C23) or 1275-1296 of SEQ ID NO:1 (C31).

In yet another embodiment of the present invention, a BoNT/A peptide useful in a method disclosed in the present specification for determining the presence or absence of an anti-BoNT/A antibody has a length of at most 60 amino acids and consisting of at least 5 contiguous amino acids selected from one of the following BoNT/A amino acid sequences: 449-467 of SEQ ID NO:1(N1), 463-481 of SEQ ID NO:1 (N2), 491-509 of SEQ ID NO:1 (N4), 505-523 of SEQ ID NO:1 (N5), 519-537 of SEQ ID NO:1 (N6), 533-551 of SEQ ID NO:1 (N7), 547-565 of SEQ ID NO:1 (N8), 561-579 of SEQ ID NO:1 (N9), 575-593 of SEQ ID NO:1 (N10), 589-607 of SEQ ID NO:1 (N11), 631-649 of SEQ ID NO:1 (N14), 645-663 of SEQ ID NO:1 (N15), 659-677 of SEQ ID NO:1 (N16), 673-691 of SEQ ID NO:1 (N17), 701-719 of SEQ ID NO:1 (N19), 715-733 of SEQ ID NO:1 (N20), 729-747 of SEQ ID NO:1 (N21), 743-761 of SEQ ID NO:1 (N22), 757-775 of SEQ ID NO:1 (N23), 771-789 of SEQ ID NO:1 (N24), 785-803 of SEQ ID NO:1 (N25), 799-817 of SEQ ID NO:1 (N26), 813-831 of SEQ ID NO:1 (N27), 827-845 of SEQ ID NO:1 (N28), 869-887 of SEQ ID NO:1 (C2), 883-901 of SEQ ID NO:1 (C3), 911-929 of SEQ ID NO:1 (C5), 925-943 of SEQ ID NO:1 (C6), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1065-1083 of SEQ ID NO:1 (C16), 1079-1097 of SEQ ID NO:1 (C17), 1107-1125 of SEQ ID NO:1 (C19), 1121-1139 of SEQ ID NO:1 (C20), 1135-1153 of SEQ ID NO:1 (C21), 1149-1167 of SEQ ID NO:1 (C22), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28), 1247-1265 of SEQ ID NO:1 (C29), 1261-1279 of SEQ ID NO:1 (C30), or 1275-1296 of SEQ ID NO:1 (C31), or a conservative variant thereof. In an aspect of this embodiment, such a BoNT/A peptide is selected from one of the following amino acid sequences: 463-481 of SEQ ID NO:1 (N2), 519-537 of SEQ ID NO:1 (N6), 533-551 of SEQ ID NO:1 (N7), 547-565 of SEQ ID NO:1 (N8), 561-579 of SEQ ID NO:1 (N9), 589-607 of SEQ ID NO:1 (N11), 659-677 of SEQ ID NO:1 (N16), 743-761 of SEQ ID NO:1 (N22), 785-803 of SEQ ID NO:1 (N25), 813-831 of SEQ ID NO:1 (N27), 827-845 of SEQ ID NO:1 (N28), 869-887 of SEQ ID NO:1 (C2), 883-901 of SEQ ID NO:1 (C3), 911-929 of SEQ ID NO:1 (C6), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1121-1139 of SEQ ID NO:1 (C20), 1135-1153 of SEQ ID NO:1 (C21), 1177-1195 of SEQ ID NO:1 (C24), 1261-1279 of SEQ ID NO:1 (C30) or 1275-1296 of SEQ ID NO:1 (C31), or a conservative variant thereof. In another aspect of this embodiment, such a BoNT/A peptide is selected from one of the following amino acid sequences: 463-481 of SEQ ID NO:1 (N2), 533-551 of SEQ ID NO:1 (N7), 547-565 of SEQ ID NO:1 (N8), 561-579 of SEQ ID NO:1 (N9), 659-677 of SEQ ID NO:1 (N16), 743-761 of SEQ ID NO:1 (N22), 785-803 of SEQ ID NO:1 (N25), 813-831 of SEQ ID NO:1 (N27), 883-901 of SEQ ID NO:1 (C3), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1121-1139 of SEQ ID NO:1 (C20) or 1177-1195 of SEQ ID NO:1 (C24) or 1275-1296 of SEQ ID NO: 1 (C31), or a conservative variant thereof. In another aspect of this embodiment, such a BoNT/A peptide is selected from one of the following amino acid sequences: 533-551 of SEQ ID NO: 1 (N7), 547-565 of SEQ ID NO: 1 (N8), 743-761 of SEQ ID NO: 1 (N22), 785-803 of SEQ ID NO: 1 (N25), 813-831 of SEQ ID NO: 1 (N27), 995-1013 of SEQ ID NO: 1 (C11); 1051-1069 of SEQ ID NO: 1 (C15), 1177-1195 of SEQ ID NO: 1 (C24), or 1275-1296 of SEQ ID NO: 1 (C31), or a conservative variant thereof.

In yet another aspect of this embodiment, a BoNT/A peptide useful in a method disclosed in the present specification for determining the presence or absence of an anti-BoNT/A antibody has a length of at most 60 amino acids and consisting of at least 5 contiguous amino acids selected from one of the following BoNT/A amino acid sequences: 463-481 of SEQ ID NO:1 (N2), 505-523 of SEQ ID NO:1 (N5), 519-537 of SEQ ID NO:1 (N6), 533-551 of SEQ ID NO:1 (N7), 603-621 of SEQ ID NO:1 (N12), 645-663 of SEQ ID NO:1 (N15), 659-677 of SEQ ID NO:1 (N16), 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1079-1097 of SEQ ID NO:1 (C17), 1107-1125 of SEQ ID NO:1 (C19), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28) or 1275-1296 of SEQ ID NO:1 (C31), or a conservative variant thereof. In yet another aspect of this embodiment, such a BoNT/A peptide is selected from one of the following amino acid sequences: 533-551 of SEQ ID NO:1 (N7), 659-677 of SEQ ID NO:1 (N16), 701-719 of SEQ ID NO:1 (N1 9), 729-747 of SEQ ID NO:1 (N21), 757-775 of SEQ ID NO:1 (N23), 799-817 of SEQ ID NO:1 (N26), 1065-1083 of SEQ ID NO:1 (C16), 1107-1125 of SEQ ID NO:1 (C19), 1163-1181 of SEQ ID NO:1 (C23), 1191-1209 of SEQ ID NO:1 (C25), 1233-1251 of SEQ ID NO:1 (C28) or 1275-1296 of SEQ ID NO:1 (C31), or a conservative variant thereof. In yet another aspect of this embodiment, such a BoNT/A peptide is selected from one of the following amino acid sequences: 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1079-1097 of SEQ ID NO:1 (C17), 1107-1125 of SEQ ID NO:1 (C19), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28) or 1275-1296 of SEQ ID NO:1 (C31), or a conservative variant thereof. In yet another aspect of this embodiment, such a BoNT/A peptide is selected from one of the following amino acid sequences: 729-747 of SEQ ID NO:1 (N21) 799-817 of SEQ ID NO:1 (N26), 1065-1083 of SEQ ID NO:1 (C16), 1163-1181 of SEQ ID NO:1 (C23) or 1275-1296 of SEQ ID NO:1 (C31), or a conservative variant thereof.

In yet another embodiment of the present invention, a BoNT/A peptide useful in a method disclosed in the present specification for determining the presence or absence of an anti-BoNT/A antibody has a length of at most 60 amino acids and consisting of at least 5 contiguous amino acids selected from one of the following BoNT/A amino acid sequences: 449-467 of SEQ ID NO:1(N1), 463-481 of SEQ ID NO:1 (N2), 491-509 of SEQ ID NO:1 (N4), 505-523 of SEQ ID NO:1 (N5), 519-537 of SEQ ID NO:1 (N6), 533-551 of SEQ ID NO:1 (N7), 547-565 of SEQ ID NO:1 (N8), 561-579 of SEQ ID NO:1 (N9), 575-593 of SEQ ID NO:1 (N10), 589-607 of SEQ ID NO:1 (N11), 631-649 of SEQ ID NO:1 (N14), 645-663 of SEQ ID NO:1 (N15), 659-677 of SEQ ID NO:1 (N16), 673-691 of SEQ ID NO:1 (N17), 701-719 of SEQ ID NO:1 (N19), 715-733 of SEQ ID NO:1 (N20), 729-747 of SEQ ID NO:1 (N21), 743-761 of SEQ ID NO:1 (N22), 757-775 of SEQ ID NO:1 (N23), 771-789 of SEQ ID NO:1 (N24), 785-803 of SEQ ID NO:1 (N25), 799-817 of SEQ ID NO:1 (N26), 813-831 of SEQ ID NO:1 (N27), 827-845 of SEQ ID NO:1 (N28), 869-887 of SEQ ID NO:1 (C2), 883-901 of SEQ ID NO:1 (C3), 911-929 of SEQ ID NO:1 (C5), 925-943 of SEQ ID NO:1 (C6), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1065-1083 of SEQ ID NO:1 (C16), 1079-1097 of SEQ ID NO:1 (C17), 1107-1125 of SEQ ID NO:1 (C19), 1121-1139 of SEQ ID NO:1 (C20), 1135-1153 of SEQ ID NO:1 (C21), 1149-1167 of SEQ ID NO:1 (C22), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28), 1247-1265 of SEQ ID NO:1 (C29), 1261-1279 of SEQ ID NO:1 (C30), or 1275-1296 of SEQ ID NO:1 (C31), or a non-conservative variant thereof. In an aspect of this embodiment, such a BoNT/A peptide is selected from one of the following amino acid sequences: 463-481 of SEQ ID NO:1 (N2), 519-537 of SEQ ID NO:1 (N6), 533-551 of SEQ ID NO:1 (N7), 547-565 of SEQ ID NO:1 (N8), 561-579 of SEQ ID NO:1 (N9), 589-607 of SEQ ID NO:1 (N11), 659-677 of SEQ ID NO:1 (N16), 743-761 of SEQ ID NO:1 (N22), 785-803 of SEQ ID NO:1 (N25), 813-831 of SEQ ID NO:1 (N27), 827-845 of SEQ ID NO:1 (N28), 869-887 of SEQ ID NO:1 (C2), 883-901 of SEQ ID NO:1 (C3), 911-929 of SEQ ID NO:1 (C6), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1121-1139 of SEQ ID NO:1 (C20), 1135-1153 of SEQ ID NO:1 (C21), 1177-1195 of SEQ ID NO:1 (C24), 1261-1279 of SEQ ID NO:1 (C30) or 1275-1296 of SEQ ID NO:1 (C31), or a non-conservative variant thereof. In another aspect of this embodiment, such a BoNT/A peptide is selected from one of the following amino acid sequences: 463-481 of SEQ ID NO:1 (N2), 533-551 of SEQ ID NO:1 (N7), 547-565 of SEQ ID NO:1 (N8), 561-579 of SEQ ID NO:1 (N9), 659-677 of SEQ ID NO:1 (N16), 743-761 of SEQ ID NO:1 (N22), 785-803 of SEQ ID NO:1 (N25), 813-831 of SEQ ID NO:1 (N27), 883-901 of SEQ ID NO:1 (C3), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1121-1139 of SEQ ID NO:1 (C20) or 1177-1195 of SEQ ID NO:1 (C24) or 1275-1296 of SEQ ID NO: 1 (C31), or a non-conservative variant thereof. In another aspect of this embodiment, such a BoNT/A peptide is selected from one of the following amino acid sequences: 533-551 of SEQ ID NO: 1 (N7), 547-565 of SEQ ID NO: 1 (N8), 743-761 of SEQ ID NO: 1 (N22), 785-803 of SEQ ID NO: 1 (N25), 813-831 of SEQ ID NO: 1 (N27), 995-1013 of SEQ ID NO: 1 (C11); 1051-1069 of SEQ ID NO: 1 (C15), 1177-1195 of SEQ ID NO: 1 (C24), or 1275-1296 of SEQ ID NO: 1 (C31), or a non-conservative variant thereof.

In yet another aspect of this embodiment, a BoNT/A peptide useful in a method disclosed in the present specification for determining the presence or absence of an anti-BoNT/A antibody has a length of at most 60 amino acids and consisting of at least 5 contiguous amino acids selected from one of the following BoNT/A amino acid sequences: 463-481 of SEQ ID NO:1 (N2), 505-523 of SEQ ID NO:1 (N5), 519-537 of SEQ ID NO:1 (N6), 533-551 of SEQ ID NO:1 (N7), 603-621 of SEQ ID NO:1 (N12), 645-663 of SEQ ID NO:1 (N15), 659-677 of SEQ ID NO:1 (N16), 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1079-1097 of SEQ ID NO:1 (C17), 1107-1125 of SEQ ID NO:1 (C19), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28) or 1275-1296 of SEQ ID NO:1 (C31), or a non-conservative variant thereof. In yet another aspect of this embodiment, such a BoNT/A peptide is selected from one of the following amino acid sequences: 533-551 of SEQ ID NO:1 (N7), 659-677 of SEQ ID NO:1 (N16), 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1107-1125 of SEQ ID NO:1 (C19), 1163-1181 of SEQ ID NO:1 (C23), 1191-1209 of SEQ ID NO:1 (C25), 1233-1251 of SEQ ID NO:1 (C28) or 1275-1296 of SEQ ID NO:1 (C31), or a non-conservative variant thereof. In yet another aspect of this embodiment, such a BoNT/A peptide is selected from one of the following amino acid sequences: 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1079-1097 of SEQ ID NO:1 (C17), 1107-1125 of SEQ ID NO:1 (C19), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28) or 1275-1296 of SEQ ID NO:1 (C31), or a non-conservative variant thereof. In yet another aspect of this embodiment, such a BoNT/A peptide is selected from one of the following amino acid sequences: 729-747 of SEQ ID NO:1 (N21) 799-817 of SEQ ID NO:1 (N26), 1065-1083 of SEQ ID NO:1 (C16), 1163-1181 of SEQ ID NO:1 (C23) or 1275-1296 of SEQ ID NO:1 (C31), or a non-conservative variant thereof.

In yet another embodiment of the present invention, a BoNT/A peptide useful in a method disclosed in the present specification for determining the presence or absence of an anti-BoNT/A antibody has a length of at most 60 amino acids and consisting of at least 5 contiguous amino acids selected from one of the following BoNT/A amino acid sequences: 449-467 of SEQ ID NO:1(N1), 463-481 of SEQ ID NO:1 (N2), 491-509 of SEQ ID NO:1 (N4), 505-523 of SEQ ID NO:1 (N5), 519-537 of SEQ ID NO:1 (N6), 533-551 of SEQ ID NO:1 (N7), 547-565 of SEQ ID NO:1 (N8), 561-579 of SEQ ID NO:1 (N9), 575-593 of SEQ ID NO:1 (N10), 589-607 of SEQ ID NO:1 (N11), 631-649 of SEQ ID NO:1 (N14), 645-663 of SEQ ID NO:1 (N15), 659-677 of SEQ ID NO:1 (N16), 673-691 of SEQ ID NO:1 (N17), 701-719 of SEQ ID NO:1 (N19), 715-733 of SEQ ID NO:1 (N20), 729-747 of SEQ ID NO:1 (N21), 743-761 of SEQ ID NO:1 (N22), 757-775 of SEQ ID NO:1 (N23), 771-789 of SEQ ID NO:1 (N24), 785-803 of SEQ ID NO:1 (N25), 799-817 of SEQ ID NO:1 (N26), 813-831 of SEQ ID NO:1 (N27), 827-845 of SEQ ID NO:1 (N28), 869-887 of SEQ ID NO:1 (C2), 883-901 of SEQ ID NO:1 (C3), 911-929 of SEQ ID NO:1 (C5), 925-943 of SEQ ID NO:1 (C6), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1065-1083 of SEQ ID NO:1 (C16), 1079-1097 of SEQ ID NO:1 (C17), 1107-1125 of SEQ ID NO:1 (C19), 1121-1139 of SEQ ID NO:1 (C20), 1135-1153 of SEQ ID NO:1 (C21), 1149-1167 of SEQ ID NO:1 (C22), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28), 1247-1265 of SEQ ID NO:1 (C29), 1261-1279 of SEQ ID NO:1 (C30), or 1275-1296 of SEQ ID NO:1 (C31), or an immunoreactive fragment thereof, with the proviso that the BoNT/A peptide is not SEQ ID NO:2. In an aspect of this embodiment, such a BoNT/A peptide is selected from one of the following amino acid sequences: 463-481 of SEQ ID NO:1 (N2), 519-537 of SEQ ID NO:1 (N6), 533-551 of SEQ ID NO:1 (N7), 547-565 of SEQ ID NO:1 (N8), 561-579 of SEQ ID NO:1 (N9), 589-607 of SEQ ID NO:1 (N11), 659-677 of SEQ ID NO:1 (N16), 743-761 of SEQ ID NO:1 (N22), 785-803 of SEQ ID NO:1 (N25), 813-831 of SEQ ID NO:1 (N27), 827-845 of SEQ ID NO:1 (N28), 869-887 of SEQ ID NO:1 (C2), 883-901 of SEQ ID NO:1 (C3), 911-929 of SEQ ID NO:1 (C6), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1121-1139 of SEQ ID NO:1 (C20), 1135-1153 of SEQ ID NO:1 (C21), 1177-1195 of SEQ ID NO:1 (C24), 1261-1279 of SEQ ID NO:1 (C30) or 1275-1296 of SEQ ID NO:1 (C31), or an immunoreactive fragment thereof, with the proviso that the BoNT/A peptide is not SEQ ID NO:2. In another aspect of this embodiment, such a BoNT/A peptide is selected from one of the following amino acid sequences: 463-481 of SEQ ID NO:1 (N2), 533-551 of SEQ ID NO:1 (N7), 547-565 of SEQ ID NO:1 (N8), 561-579 of SEQ ID NO:1 (N9), 659-677 of SEQ ID NO:1 (N16), 743-761 of SEQ ID NO:1 (N22), 785-803 of SEQ ID NO:1 (N25), 813-831 of SEQ ID NO:1 (N27), 883-901 of SEQ ID NO:1 (C3), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1121-1139 of SEQ ID NO:1 (C20) or 1177-1195 of SEQ ID NO:1 (C24) or 1275-1296 of SEQ ID NO: 1 (C31), or an immunoreactive fragment thereof, with the proviso that the BoNT/A peptide is not SEQ ID NO:2. In another aspect of this embodiment, such a BoNT/A peptide is selected from one of the following amino acid sequences: 533-551 of SEQ ID NO: 1 (N7), 547-565 of SEQ ID NO: 1 (N8), 743-761 of SEQ ID NO: 1 (N22), 785-803 of SEQ ID NO: 1 (N25), 813-831 of SEQ ID NO: 1 (N27), 995-1013 of SEQ ID NO: 1 (C11); 1051-1069 of SEQ ID NO: 1 (C15), 1177-1195 of SEQ ID NO: 1 (C24), or 1275-1296 of SEQ ID NO: 1 (C31), or an immunoreactive fragment thereof, with the proviso that the BoNT/A peptide is not SEQ ID NO:2.

In yet another aspect of this embodiment, a BoNT/A peptide useful in a method disclosed in the present specification for determining the presence or absence of an anti-BoNT/A antibody has a length of at most 60 amino acids and consisting of at least 5 contiguous amino acids selected from one of the following BoNT/A amino acid sequences: 463-481 of SEQ ID NO:1 (N2), 505-523 of SEQ ID NO:1 (N5), 519-537 of SEQ ID NO:1 (N6), 533-551 of SEQ ID NO:1 (N7), 603-621 of SEQ ID NO:1 (N12), 645-663 of SEQ ID NO:1 (N15), 659-677 of SEQ ID NO:1 (N16), 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1079-1097 of SEQ ID NO:1 (C17), 1107-1125 of SEQ ID NO:1 (C19), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28) or 1275-1296 of SEQ ID NO:1 (C31), or an immunoreactive fragment thereof, with the proviso that the BoNT/A peptide is not SEQ ID NO:2. In yet another aspect of this embodiment, such a BoNT/A peptide is selected from one of the following amino acid sequences: 533-551 of SEQ ID NO:1 (N7), 659-677 of SEQ ID NO:1 (N16), 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1107-1125 of SEQ ID NO:1 (C19), 1163-1181 of SEQ ID NO:1 (C23), 1191-1209 of SEQ ID NO:1 (C25), 1233-1251 of SEQ ID NO:1 (C28) or 1275-1296 of SEQ ID NO:1 (C31), or an immunoreactive fragment thereof, with the proviso that the BoNT/A peptide is not SEQ ID NO:2. In yet another aspect of this embodiment, such a BoNT/A peptide is selected from one of the following amino acid sequences: 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1079-1097 of SEQ ID NO:1 (C17), 1107-1125 of SEQ ID NO:1 (C19), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28) or 1275-1296 of SEQ ID NO:1 (C31), or an immunoreactive fragment thereof, with the proviso that the BoNT/A peptide is not SEQ ID NO:2. In yet another aspect of this embodiment, such a BoNT/A peptide is selected from one of the following amino acid sequences: 729-747 of SEQ ID NO:1 (N21) 799-817 of SEQ ID NO:1 (N26), 1065-1083 of SEQ ID NO:1 (C16), 1163-1181 of SEQ ID NO:1 (C23) or 1275-1296 of SEQ ID NO:1 (C31), or an immunoreactive fragment thereof, with the proviso that the BoNT/A peptide is not SEQ ID NO:2.

In is envisioned that a BoNT/A peptide useful in a method disclosed in the present specification for determining the presence or absence of an anti-BoNT/A antibody can have any of a variety of lengths from at least 5 amino acids to at most 60 amino acids. Therefore, aspects of this embodiment may include a BoNT/A peptide with at least, e.g., five amino acids, six amino acids, seven amino acids, eight amino acids, nine amino acids, ten amino acids, 11 amino acids, 12 amino acids, 13 amino acids, 14 amino acids, 15 amino acids, 16 amino acids, 17 amino acids, 18 amino acids, 19 amino acids, 20 amino acids, 25 amino acids, 30 amino acids, 35 amino acids, 40 amino acids, 45 amino acids, 50 amino acids, 55 amino acids or 60 amino acids. Other aspects of this embodiment may include a BoNT/A peptide with at least, e.g., five amino acids of SEQ ID NO:1, six amino acids of SEQ ID NO:1, seven amino acids of SEQ ID NO:1, eight amino acids of SEQ ID NO:1, nine amino acids of SEQ ID NO:1, ten amino acids of SEQ ID NO:1, 11 amino acids of SEQ ID NO:1, 12 amino acids of SEQ ID NO:1, 13 amino acids of SEQ ID NO:1, 14 amino acids of SEQ ID NO:1, 15 amino acids of SEQ ID NO:1, 16 amino acids of SEQ ID NO:1, 17 amino acids of SEQ ID NO:1, 18 amino acids of SEQ ID NO:1, 19 amino acids of SEQ ID NO:1, 20 amino acids of SEQ ID NO:1, 25 amino acids of SEQ ID NO:1, 30 amino acids of SEQ ID NO:1, 35 amino acids of SEQ ID NO:1, 40 amino acids of SEQ ID NO:1, 45 amino acids of SEQ ID NO:1, 50 amino acids of SEQ ID NO:1, 55 amino acids or 60 amino acids of SEQ ID NO:1. In further embodiments, such a BoNT/A peptide of the invention may include a BoNT/A peptide with at least, e.g., five amino acids, six amino acids, seven amino acids, eight amino acids, nine amino acids, ten amino acids, 11 amino acids, 12 amino acids, 13 amino acids, 14 amino acids, 15 amino acids, 16 amino acids, 17 amino acids, 18 amino acids, 19 amino acids, 20 amino acids, 25 amino acids, 30 amino acids, 35 amino acids, 40 amino acids, 45 amino acids, 50 amino acids, 55 amino acids or 60 amino acids and consist of at least 5 contiguous amino acids selected from one of the following BoNT/A amino acid sequences: 449-467 of SEQ ID NO:1(N1), 463-481 of SEQ ID NO:1 (N2), 491-509 of SEQ ID NO:1 (N4), 505-523 of SEQ ID NO:1 (N5), 519-537 of SEQ ID NO:1 (N6), 533-551 of SEQ ID NO:1 (N7), 547-565 of SEQ ID NO:1 (N8), 561-579 of SEQ ID NO:1 (N9), 575-593 of SEQ ID NO:1 (N10), 589-607 of SEQ ID NO:1 (N11), 631-649 of SEQ ID NO:1 (N14), 645-663 of SEQ ID NO:1 (N15), 659-677 of SEQ ID NO:1 (N16), 673-691 of SEQ ID NO:1 (N17), 701-719 of SEQ ID NO:1 (N19), 715-733 of SEQ ID NO:1 (N20), 729-747 of SEQ ID NO:1 (N21), 743-761 of SEQ ID NO:1 (N22), 757-775 of SEQ ID NO:1 (N23), 771-789 of SEQ ID NO:1 (N24), 785-803 of SEQ ID NO:1 (N25), 799-817 of SEQ ID NO:1 (N26), 813-831 of SEQ ID NO:1 (N27), 827-845 of SEQ ID NO:1 (N28), 869-887 of SEQ ID NO:1 (C2), 883-901 of SEQ ID NO:1 (C3), 911-929 of SEQ ID NO:1 (C5), 925-943 of SEQ ID NO:1 (C6), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1065-1083 of SEQ ID NO:1 (C16), 1079-1097 of SEQ ID NO:1 (C17), 1107-1125 of SEQ ID NO:1 (C19), 1121-1139 of SEQ ID NO:1 (C20), 1135-1153 of SEQ ID NO:1 (C21), 1149-1167 of SEQ ID NO:1 (C22), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28), 1247-1265 of SEQ ID NO:1 (C29), 1261-1279 of SEQ ID NO:1 (C30) or 1275-1296 of SEQ ID NO:1 (C31), or a conservative variant, a non-conservative variant, or immunoreactive fragment thereof, with the proviso that the BoNT/A peptide is not SEQ ID NO:2.

In another embodiment of the present invention, a BoNT/A peptide useful in a method disclosed in the present specification for determining the presence or absence of an anti-BoNT/A antibody can comprise one BoNT/A peptide disclosed in the present specification. In another embodiment of the present invention, a BoNT/A peptide useful in a method disclosed in the present specification for determining the presence or absence of an anti-BoNT/A antibody can comprise a plurality of BoNT/A peptides disclosed in the present specification. Thus, aspects of this embodiment can include one or more BoNT/A peptides, two or more BoNT/A peptides, three or more BoNT/A peptides, four or more BoNT/A peptides, five or more BoNT/A peptides, six or more BoNT/A peptides, seven or more BoNT/A peptides, eight or more BoNT/A peptides, nine or more BoNT/A peptides, ten or more BoNT/A peptides, 15 or more BoNT/A peptides, 20 or more BoNT/A peptides, 25 or more BoNT/A peptides or 30 or more BoNT/A peptides. In other aspects of this embodiment can include one or more BoNT/A conservative variants, two or more BoNT/A conservative variants, three or more BoNT/A conservative variants, four or more BoNT/A conservative variants, five or more BoNT/A conservative variants, six or more BoNT/A conservative variants, seven or more BoNT/A conservative variants, eight or more BoNT/A conservative variants, nine or more BoNT/A conservative variants, ten or more BoNT/A conservative variants, 15 or more BoNT/A conservative variants, 20 or more BoNT/A conservative variants, 25 or more BoNT/A conservative variants or 30 or more BoNT/A conservative variants. In further aspects of this embodiment can include one or more BoNT/A non-conservative variants, two or more BoNT/A non-conservative variants, three or more BoNT/A non-conservative variants, four or more BoNT/A non-conservative variants, five or more BoNT/A non-conservative variants, six or more BoNT/A non-conservative variants, seven or more BoNT/A non-conservative variants, eight or more BoNT/A non-conservative variants, nine or more BoNT/A non-conservative variants, ten or more BoNT/A non-conservative variants, 15 or more BoNT/A non-conservative variants, 20 or more BoNT/A non-conservative variants, 25 or more BoNT/A non-conservative variants or 30 or more BoNT/A non-conservative variants. In still other aspects of this embodiment can include one or more BoNT/A immunoreactive fragments, two or more BoNT/A immunoreactive fragments, three or more BoNT/A immunoreactive fragments, four or more BoNT/A immunoreactive fragments, five or more BoNT/A immunoreactive fragments, six or more BoNT/A immunoreactive fragments, seven or more BoNT/A immunoreactive fragments, eight or more BoNT/A immunoreactive fragments, nine or more BoNT/A peptides, ten or more BoNT/A immunoreactive fragments, 15 or more BoNT/A immunoreactive fragments, 20 or more BoNT/A immunoreactive fragments, 25 or more BoNT/A immunoreactive fragments or 30 or more BoNT/A immunoreactive fragments. BoNT/A peptides disclosed in the present specification useful for determining the presence or absence of an anti-BoNT/A antibody can be selected, for example, depending on immunological factors, such as potency of the peptide in inducing an immune response, and technical factors, such as chemical synthesis yields. It is also understood that the two or more BoNT/A peptides can be provided separately or as part of a compound molecule such as a chimeric peptide or heterologous protein.

In an aspect of this embodiment, a method of determining the presence or absence of a BoNT/A antibody uses two or more immunoreactive BoNT/A peptides selected from the following amino acid sequences: 533-551 of SEQ ID NO:1 (N7), 547-565 of SEQ ID NO:1 (N8), 743-761 of SEQ ID NO:1 (N22), 785-803 of SEQ ID NO:1 (N25), 813-831 of SEQ ID NO:1 (N27), 995-1013 of SEQ ID NO:1 (C11); 1051-1069 of SEQ ID NO:1 (C15), 1177-1195 of SEQ ID NO:1 (C24), and 1275-1296 of SEQ ID NO:1 (C31), or a conservative variant, a non-conservative variant or an immunoreactive fragment thereof, with the proviso that the BoNT/A peptide is not SEQ ID NO:2. In another aspect of this embodiment, one of the selected amino acid sequence is 533-551 of SEQ ID NO:1 (N8) or a conservative variant, a non-conservative variant or an immunoreactive fragment thereof, with the proviso that the BoNT/A peptide is not SEQ ID NO:2. In yet another aspect of this embodiment, the following two amino acid sequences are selected: 533-551 of SEQ ID NO:1 (N8) and 981-999 of SEQ ID NO:1 (C10), or a conservative variant, a non-conservative variant or an immunoreactive fragment thereof, with the proviso that the BoNT/A peptide is not SEQ ID NO:2. In yet another aspect of this embodiment, the following three amino acid sequences are selected: 533-551 of SEQ ID NO:1 (N8), 981-999 of SEQ ID NO:1 (C10) and 1051-1069 of SEQ ID NO:1 (C15), or a conservative variant, a non-conservative variant or an immunoreactive fragment thereof, with the proviso that the BoNT/A peptide is not SEQ ID NO:2. In a further aspect of this embodiment, one of the amino acid sequences selected is 785-803 of SEQ ID NO:1 (N25) or a conservative variant, a non-conservative variant or an immunoreactive fragment thereof, with the proviso that the BoNT/A peptide is not SEQ ID NO:2. In a still further aspect of this embodiment, the following two amino acid sequences are selected: 785-803 of SEQ ID NO:1 (N25) and 981-999 of SEQ ID NO:1 (C10), or a conservative variant, a non-conservative variant or an immunoreactive fragment thereof, with the proviso that the BoNT/A peptide is not SEQ ID NO:2. In a still further aspect of this embodiment, the following three amino acid sequences are selected: 785-803 of SEQ ID NO:1 (N25), 981-999 of SEQ ID NO:1 (C10) and 1051-1069 of SEQ ID NO:1 (C15), or a conservative variant, a non-conservative variant or an immunoreactive fragment thereof, with the proviso that the BoNT/A peptide is not SEQ ID NO:2. In an additional aspect of this embodiment, one of the amino acid sequences selected is 813-831 of SEQ ID NO:1 (N27) or a conservative variant, a non-conservative variant or an immunoreactive fragment thereof, with the proviso that the BoNT/A peptide is not SEQ ID NO:2. In another additional aspect of this embodiment, the following two amino acid sequences are selected: 813-831 of SEQ ID NO:1 (N27) and 981-999 of SEQ ID NO:1 (C10), or a conservative variant, a non-conservative variant or an immunoreactive fragment thereof, with the proviso that the BoNT/A peptide is not SEQ ID NO:2. In another additional aspect of this embodiment, the following three amino acid sequences are selected: 813-831 of SEQ ID NO:1 (N27), 981-999 of SEQ ID NO:1 (C10) and 1051-1069 of SEQ ID NO:1 (C15), or a conservative variant, a non-conservative variant or an immunoreactive fragment thereof, with the proviso that the BoNT/A peptide is not SEQ ID NO:2.

In an aspect of this embodiment, a method of determining the presence or absence of a BoNT/A antibody uses two or more immunoreactive BoNT/A peptides selected from the following amino acid sequences: 659-677 of SEQ ID NO:1 (N16), 729-747 of SEQ ID NO:1 (N21), 799-817 of SEQ ID NO:1 (N26), 1065-1083 of SEQ ID NO:1 (C16), 1163-1181 of SEQ ID NO:1 (C23), and 1275-1296 of SEQ ID NO:1 (C31), or a conservative variant, a non-conservative variant or an immunoreactive fragment thereof, with the proviso that the BoNT/A peptide is not SEQ ID NO:2. In another aspect of this embodiment, one of the amino acid sequences selected is 1065-1083 of SEQ ID NO:1 (C16) or a conservative variant, a non-conservative variant or an immunoreactive fragment thereof, with the proviso that the BoNT/A peptide is not SEQ ID NO:2. In yet another aspect of this embodiment, the following two amino acid sequences are selected: 1065-1083 of SEQ ID NO:1 (C16) and 1163-1181 of SEQ ID NO:1 (C23), or a conservative variant, a non-conservative variant or an immunoreactive fragment thereof, with the proviso that the BoNT/A peptide is not SEQ ID NO:2. In yet another aspect of this embodiment, the following three amino acid sequences are selected: 1065-1083 of SEQ ID NO:1 (C16), 1163-1181 of SEQ ID NO:1 (C23) and 1275-1296 of SEQ ID NO:1 (C31), or a conservative variant, a non-conservative variant or an immunoreactive fragment thereof, with the proviso that the BoNT/A peptide is not SEQ ID NO:2. In a further aspect of this embodiment, one of the amino acid sequences selected is 799-817 of SEQ ID NO:1 (N26) or a conservative variant, a non-conservative variant or an immunoreactive fragment thereof, with the proviso that the BoNT/A peptide is not SEQ ID NO:2. In a still further aspect of this embodiment, the following two amino acid sequences are selected: 799-817 of SEQ ID NO:1 (N26) and 1065-1083 of SEQ ID NO:1 (C16), or a conservative variant, a non-conservative variant or an immunoreactive fragment thereof, with the proviso that the BoNT/A peptide is not SEQ ID NO:2. In a still further aspect of this embodiment, the following three amino acid sequences are selected: 799-817 of SEQ ID NO:1 (N26), 1065-1083 of SEQ ID NO:1 (C16) and 1163-1181 of SEQ ID NO:1 (C23), or a conservative variant, a non-conservative variant or an immunoreactive fragment thereof, with the proviso that the BoNT/A peptide is not SEQ ID NO:2. In an additional aspect of this embodiment, one of the amino acid sequences selected is 729-747 of SEQ ID NO:1 (N21) or a conservative variant, a non-conservative variant or an immunoreactive fragment thereof, with the proviso that the BoNT/A peptide is not SEQ ID NO:2. In another additional aspect of this embodiment, the following two amino acid sequences are selected: 729-747 of SEQ ID NO:1 (N21) and 1065-1083 of SEQ ID NO:1 (C16), or a conservative variant, a non-conservative variant or an immunoreactive fragment thereof, with the proviso that the BoNT/A peptide is not SEQ ID NO:2. In another additional aspect of this embodiment, the following three amino acid sequences are selected: 729-747 of SEQ ID NO:1 (N21), 1065-1083 of SEQ ID NO:1 (C16) and 1163-1181 of SEQ ID NO:1 (C23), or a conservative variant, a non-conservative variant or an immunoreactive fragment thereof, with the proviso that the BoNT/A peptide is not SEQ ID NO:2.

It is also envisioned that any and all combinations of BoNT/A peptides disclosed in the specification can be useful for determining the presence or absence of a BoNT/A antibody, including, e.g., BoNT/A peptides of SEQ ID NO:1, BoNT/A conservative variants, BoNT/A non-conservative variants and BoNT/A immunoreactive fragments. Thus, aspects of this embodiment include one or more BoNT/A peptides comprising one or more BoNT/A peptides of SEQ ID NO: 1 and one or more BoNT/A conservative variants; one or more BoNT/A peptides of SEQ ID NO: 1 and one or more BoNT/A non-conservative variants; one or more BoNT/A peptides of SEQ ID NO: 1 and one or more BoNT/A immunoreactive fragments; one or more BoNT/A conservative variants and one or more BoNT/A non-conservative variants; one or more BoNT/A conservative variants and one or more BoNT/A immunoreactive fragments; one or more BoNT/A non-conservative variants and one or more BoNT/A immunoreactive fragments; one or more BoNT/A peptides of SEQ ID NO: 1, one or more BoNT/A conservative variants and one or more BoNT/A non-conservative variants; one or more BoNT/A peptides of SEQ ID NO: 1, one or more BoNT/A conservative variants and one or more BoNT/A immunoreactive fragments; one or more BoNT/A peptides of SEQ ID NO: 1, one or more BoNT/A non-conservative variants and one or more BoNT/A immunoreactive fragments; one or more BoNT/A conservative variants, one or more BoNT/A non-conservative variants and one or more BoNT/A immunoreactive fragments; or one or more BoNT/A peptides of SEQ ID NO: 1, one or more BoNT/A conservative variants, one or more BoNT/A non-conservative variants and one or more BoNT/A immunoreactive fragments.

Any of the above methods of the invention can be practiced, if desired, by selectively determining the presence or absence in the individual of IgG antibodies immunoreactive with each of the amino acid sequences. Any of a variety of means can be used to determine the presence or absence of antibodies immunoreactive with each of the specified amino acid sequences including, yet not limited to, enzyme-linked immunosorbent assays and radioimmunoassays, see e.g., MOLECULAR CLONING, A LABORATORY MANUAL, supra, 2001; and CURRENT PROTOCOLS IN MOLECULAR BIOLOGY, supra, 2004. In one embodiment, the botulinum toxin therapy is BoNT/A therapy.

A variety of assays are useful in a method of the invention for determining the presence or absence of antibodies immunoreactive with a BoNT/A peptide including, without limitation, enzyme-linked immunosorbent assays and radioimmunoassays, see e.g., MOLECULAR CLONING, A LABORATORY MANUAL, supra, 2001; and CURRENT PROTOCOLS IN MOLECULAR BIOLOGY, supra, 2004. The methods of the invention can be useful for predicting or determining immunoresistance to any of a variety of botulinum toxin therapies including, but not limited to, BOTOX® therapy.

The term “immunoresistance,” as used herein in reference to botulinum toxin therapy, means a reduction in beneficial effect of botulinum toxin therapy in a human or other mammal resulting from the presence in the human or other mammal of antibodies that bind to botulinum toxin. As used herein, the term “botulinum toxin therapy” means administration to a human or other mammal one or more controlled doses of botulinum toxin to obtain a beneficial therapeutic or cosmetic effect. The term botulinum toxin therapy encompasses, without limitation, the use of any naturally occurring or modified or engineered form of a botulinum toxin or a domain or fragment thereof, in any formulation, combined with any carrier or active ingredient and administered by any route of administration. An exemplary well-known botulinum toxin therapy is BOTOX® therapy. Appropriate therapeutic and cosmetic uses of botulinum toxin therapy are known in the art as discussed above.

A variety of assay formats employing one or more BoNT/A peptides of the invention can be used to determine the presence or absence of antibodies immunoreactive with a BoNT/A and, therefore, to predict or determine imunoresistance to botulinum toxin therapy according to a method of the invention. Such assay formats generally involve detecting an antigen-antibody interaction. Non-limiting examples include radioimmunoassays, enzyme immunoassays, fluorescence immunoassays, luminescent immunoassays and other nonradioisotopic assay formats. Non-competitive assays can be performed, for example, by attaching one or more selected BoNT/A peptides to a solid support; adding a test specimen; adding a secondary antibody, which is an antibody selective for the test antibody; and detecting the secondary antibody, typically by a physical property or enzymatic activity of the secondary antibody. In such an assay, the amount of signal that is detected can be proportional to the amount of antibodies which are immunoreactive with the one or more BoNT/A peptides and are present in the test specimen.

As a further non-limiting example, a competitive assay can be performed by attaching one or more selected BoNT/A peptides to a solid support; adding simultaneously a test specimen and an enzyme-labeled secondary antibody; and adding a substrate that produces a detectable compound when acted upon by the enzyme. In this type of assay format, the amount of signal that is detected is inversely proportional to the amount of BoNT antibody present in the test specimen.

In any assay format selected, a BoNT/A peptide disclosed in the specification optionally can be attached to a solid support. Such a solid support can be, without limitation, a tube, plate, column, particle or bead. The solid support selected can have a physical property that renders it readily separable from soluble or unbound material and generally allows unbound materials, such as unbound antibodies, to be washed away or otherwise removed from support-bound antibodies.

In one embodiment, the presence or absence of anti-BoNT/A antibodies immunoreactive with a BoNT/A peptide is determined using an enzyme-linked immunosorbent assay (ELISA). In another embodiment, the presence or absence of antibodies immunoreactive with a BoNT/A peptide is determined using a radioimmunoassay.

It is understood that a method disclosed in the spresent specification for determining immunoresistance to botulinum toxin therapy can be determined using a test specimen obtained from a human or other mammal prior to receipt of botulinum toxin therapy, after a single botulinum toxin treatment, after multiple botulinum toxin treatments, or after onset of resistance to botulinum toxin therapy. Useful test specimens include, but are not limited to, blood, plasma and serum. It further is understood that a method of the invention can be used to predict the likelihood of a human or other mammal developing immunoresistance or to confirm that the presence of anti-BoNT/A antibodies are a cause underlying resistance to botulinum toxin therapy.

VII. Methods of Preventing or Reducing BoNT/A Immunoresistance

As described above, patients treated with botulinum toxin can develop immunoresistance to the therapeutic treatment, reducing or eliminating the beneficial effect of botulinum toxin therapy. Methods that prevent or reduce the development of a BoNT/A-specific immune response in a human or other mammal, which in turn can prevent or reduce immunoresistance to a botulinum toxin therapy, are of major importance. These treatments would allow for 1) the suppression of a potential deleterious immune response in a patient undergoing BoNT/A therapy thereby affording a more prolonged treatment course relative to current therapies; 2) the suppression of a BoNT/A immunoresponsive state in a patient thereby offering additional treatments that would otherwise have been ineffective. Therefore, these assays present a major benefit in terms of providing better patient care and reducing health care costs. The BoNT/A peptides disclosed in the present specification are useful in methods of determining immunoresistance to botulinum toxin therapy in a human or other mammal. These peptides each contain one or more epitopes recognized by antibodies contained in antisera from animals immunized with BoNT/A, and thus can serve as binding substrates for anti-BoNT/A antibodies. The methods disclosed in the present specification can be useful for preventing or reducing immunoresistance to any of a variety of botulinum toxin therapies including, but not limited to, BoNT/A therapy.

Thus, the present invention provides a method of preventing or reducing immunoresistance to botulinum toxin therapy in a human or other mammal by administering to a human or other mammal a tolerogizing composition disclosed in the present specification comprising a tolerogizing agent and a BoNT/A peptide composition disclosed in the present specification. A tolerogizing composition can be administered to a human or other mammal prior to administering botulinum toxin therapy to prevent the development of immunoresistance, during a course of botulinum toxin therapy, or after onset of immunoresistance, for example, when symptoms of resistance are first apparent. In addition, a tolerogizing composition can be administered to a human or other mammal who is at increased risk for immunoresistance to botulinum toxin therapy. Those skilled in the art will be able to determine an appropriate candidate for receiving a tolerogizing composition of the invention based on, for example, the particular condition to be treated and the presence or likelihood of symptoms of immunoresistance. In one embodiment, a method of the present invention is practiced by administering a tolerogizing composition prior to the human or other mammal receiving a BoNT/A therapy. Such a human or other mammal can be, for example, an individual at increased risk for developing immunoresistance to botulinum toxin therapy. In another embodiment, a method of the present invention is practiced by administering a tolerogizing composition after the human or other mammal has recieved a BoNT/A therapy. In yet another embodiment, a method of the present invention is practiced by administering a tolerogizing composition to a human or other mammal who has not been diagnosed with a BoNT/A immunoresistance condition. In yet another embodiment, a method of the present invention is practiced by administering a tolerogizing composition to a human or other mammal who has been diagnosed with a BoNT/A immunoresistance condition.

In an embodiment of the present invention, a BoNT/A peptide useful in a method disclosed in the present specification for preventing or reducing development of a BoNT-specific immune response has a length of at most 60 amino acids and consisting of at least 5 contiguous amino acids selected from one of the following BoNT/A amino acid sequences: 449-467 of SEQ ID NO:1(N1), 463-481 of SEQ ID NO:1 (N2), 491-509 of SEQ ID NO:1 (N4), 505-523 of SEQ ID NO:1 (N5), 519-537 of SEQ ID NO:1 (N6), 533-551 of SEQ ID NO:1 (N7), 547-565 of SEQ ID NO:1 (N8), 561-579 of SEQ ID NO:1 (N9), 575-593 of SEQ ID NO:1 (N10), 589-607 of SEQ ID NO:1 (N11), 631-649 of SEQ ID NO:1 (N14), 645-663 of SEQ ID NO:1 (N15), 659-677 of SEQ ID NO:1 (N16), 673-691 of SEQ ID NO:1 (N17), 701-719 of SEQ ID NO:1 (N19), 715-733 of SEQ ID NO:1 (N20), 729-747 of SEQ ID NO:1 (N21), 743-761 of SEQ ID NO:1 (N22), 757-775 of SEQ ID NO:1 (N23), 771-789 of SEQ ID NO:1 (N24), 785-803 of SEQ ID NO:1 (N25), 799-817 of SEQ ID NO:1 (N26), 813-831 of SEQ ID NO:1 (N27), 827-845 of SEQ ID NO:1 (N28), 869-887 of SEQ ID NO:1 (C2), 883-901 of SEQ ID NO:1 (C3), 911-929 of SEQ ID NO:1 (C5), 925-943 of SEQ ID NO:1 (C6), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1065-1083 of SEQ ID NO:1 (C16), 1079-1097 of SEQ ID NO:1 (C17),1107-1125 of SEQ ID NO:1 (C19), 1121-1139 of SEQ ID NO:1 (C20), 1135-1153 of SEQ ID NO:1 (C21), 1149-1167 of SEQ ID NO:1 (C22), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28), 1247-1265 of SEQ ID NO:1 (C29), 1261-1279 of SEQ ID NO:1 (C30), or 1275-1296 of SEQ ID NO:1 (C31), or a conservative variant or immunoreactive fragment thereof, with the proviso that the BoNT/A peptide is not SEQ ID NO:2.

In another embodiment of the present invention, a BoNT/A peptide useful in a method disclosed in the present specification for preventing or reducing development of a BoNT-specific immune response has a length of at most 60 amino acids and consisting of at least 5 contiguous amino acids selected from one of the following BoNT/A amino acid sequences: 449-467 of SEQ ID NO:1(N1), 463-481 of SEQ ID NO:1 (N2), 491-509 of SEQ ID NO:1 (N4), 505-523 of SEQ ID NO:1 (N5), 519-537 of SEQ ID NO:1 (N6), 533-551 of SEQ ID NO:1 (N7), 547-565 of SEQ ID NO:1 (N8), 561-579 of SEQ ID NO:1 (N9), 575-593 of SEQ ID NO:1 (N10), 589-607 of SEQ ID NO:1 (N11), 631-649 of SEQ ID NO:1 (N14), 645-663 of SEQ ID NO:1 (N15), 659-677 of SEQ ID NO:1 (N16), 673-691 of SEQ ID NO:1 (N17), 701-719 of SEQ ID NO:1 (N19), 715-733 of SEQ ID NO:1 (N20), 729-747 of SEQ ID NO:1 (N21), 743-761 of SEQ ID NO:1 (N22), 757-775 of SEQ ID NO:1 (N23), 771-789 of SEQ ID NO:1 (N24), 785-803 of SEQ ID NO:1 (N25), 799-817 of SEQ ID NO:1 (N26), 813-831 of SEQ ID NO:1 (N27), 827-845 of SEQ ID NO:1 (N28), 869-887 of SEQ ID NO:1 (C2), 883-901 of SEQ ID NO:1 (C3), 911-929 of SEQ ID NO:1 (C5), 925-943 of SEQ ID NO:1 (C6), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1065-1083 of SEQ ID NO:1 (C16), 1079-1097 of SEQ ID NO:1 (C17), 1107-1125 of SEQ ID NO:1 (C19), 1121-1139 of SEQ ID NO:1 (C20), 1135-1153 of SEQ ID NO:1 (C21), 1149-1167 of SEQ ID NO:1 (C22), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28), 1247-1265 of SEQ ID NO:1 (C29), 1261-1279 of SEQ ID NO:1 (C30), or 1275-1296 of SEQ ID NO:1 (C31). In an aspect of this embodiment, such a BoNT/A peptide is selected from one of the following amino acid sequences: 463-481 of SEQ ID NO:1 (N2), 519-537 of SEQ ID NO:1 (N6), 533-551 of SEQ ID NO:1 (N7), 547-565 of SEQ ID NO:1 (N8), 561-579 of SEQ ID NO:1 (N9), 589-607 of SEQ ID NO:1 (N11), 659-677 of SEQ ID NO:1 (N16), 743-761 of SEQ ID NO:1 (N22), 785-803 of SEQ ID NO:1 (N25), 813-831 of SEQ ID NO:1 (N27), 827-845 of SEQ ID NO:1 (N28), 869-887 of SEQ ID NO:1 (C2), 883-901 of SEQ ID NO:1 (C3), 911-929 of SEQ ID NO:1 (C6), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1121-1139 of SEQ ID NO:1 (C20), 1135-1153 of SEQ ID NO:1 (C21), 1177-1195 of SEQ ID NO:1 (C24), 1261-1279 of SEQ ID NO:1 (C30) or 1275-1296 of SEQ ID NO:1 (C31). In another aspect of this embodiment, such a BoNT/A peptide is selected from one of the following amino acid sequences: 463-481 of SEQ ID NO:1 (N2), 533-551 of SEQ ID NO:1 (N7), 547-565 of SEQ ID NO:1 (N8), 561-579 of SEQ ID NO:1 (N9), 659-677 of SEQ ID NO:1 (N16), 743-761 of SEQ ID NO:1 (N22), 785-803 of SEQ ID NO:1 (N25), 813-831 of SEQ ID NO:1 (N27), 883-901 of SEQ ID NO:1 (C3), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1121-1139 of SEQ ID NO:1 (C20) or 1177-1195 of SEQ ID NO:1 (C24) or 1275-1296 of SEQ ID NO: 1 (C31). In another aspect of this embodiment, such a BoNT/A peptide is selected from one of the following amino acid sequences: 533-551 of SEQ ID NO: 1 (N7), 547-565 of SEQ ID NO: 1 (N8), 743-761 of SEQ ID NO: 1 (N22), 785-803 of SEQ ID NO: 1 (N25), 813-831 of SEQ ID NO: 1 (N27), 995-1013 of SEQ ID NO: 1 (C11); 1051-1069 of SEQ ID NO: 1 (C15), 1177-1195 of SEQ ID NO: 1 (C24), or 1275-1296 of SEQ ID NO: 1 (C31).

In yet another aspect of this embodiment, a BoNT/A peptide useful in a method disclosed in the present specification for preventing or reducing development of a BoNT-specific immune response has a length of at most 60 amino acids and consisting of at least 5 contiguous amino acids selected from one of the following BoNT/A amino acid sequences: 463-481 of SEQ ID NO:1 (N2), 505-523 of SEQ ID NO:1 (N5), 519-537 of SEQ ID NO:1 (N6), 533-551 of SEQ ID NO:1 (N7), 603-621 of SEQ ID NO:1 (N12), 645-663 of SEQ ID NO:1 (N15), 659-677 of SEQ ID NO:1 (N16), 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1079-1097 of SEQ ID NO:1 (C17), 1107-1125 of SEQ ID NO:1 (C19), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28) or 1275-1296 of SEQ ID NO:1 (C31). In yet another aspect of this embodiment, such a BoNT/A peptide is selected from one of the following amino acid sequences: 533-551 of SEQ ID NO:1 (N7), 659-677 of SEQ ID NO:1 (N16), 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1107-1125 of SEQ ID NO:1 (C19), 1163-1181 of SEQ ID NO:1 (C23), 1191-1209 of SEQ ID NO:1 (C25), 1233-1251 of SEQ ID NO:1 (C28) or 1275-1296 of SEQ ID NO:1 (C31). In yet another aspect of this embodiment, such a BoNT/A peptide is selected from one of the following amino acid sequences: 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1079-1097 of SEQ ID NO:1 (C17), 1107-1125 of SEQ ID NO:1 (C19), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28) or 1275-1296 of SEQ ID NO:1 (C31). In yet another aspect of this embodiment, such a BoNT/A peptide is selected from one of the following amino acid sequences: 729-747 of SEQ ID NO:1 (N21) 799-817 of SEQ ID NO:1 (N26), 1065-1083 of SEQ ID NO:1 (C16), 1163-1181 of SEQ ID NO:1 (C23) or 1275-1296 of SEQ ID NO:1 (C31).

In yet another embodiment of the present invention, a BoNT/A peptide useful in a method disclosed in the present specification for preventing or reducing development of a BoNT-specific immune response is selected from one of the following BoNT/A amino acid sequences: 449-467 of SEQ ID NO:1(N1), 463-481 of SEQ ID NO:1 (N2), 491-509 of SEQ ID NO:1 (N4), 505-523 of SEQ ID NO:1 (N5), 519-537 of SEQ ID NO:1 (N6), 533-551 of SEQ ID NO:1 (N7), 547-565 of SEQ ID NO:1 (N8), 561-579 of SEQ ID NO:1 (N9), 575-593 of SEQ ID NO:1 (N10), 589-607 of SEQ ID NO:1 (N11), 631-649 of SEQ ID NO:1 (N14), 645-663 of SEQ ID NO:1 (N15), 659-677 of SEQ ID NO:1 (N16), 673-691 of SEQ ID NO:1 (N17), 701-719 of SEQ ID NO:1 (N19), 715-733 of SEQ ID NO:1 (N20), 729-747 of SEQ ID NO:1 (N21), 743-761 of SEQ ID NO:1 (N22), 757-775 of SEQ ID NO:1 (N23), 771-789 of SEQ ID NO:1 (N24), 785-803 of SEQ ID NO:1 (N25), 799-817 of SEQ ID NO:1 (N26), 813-831 of SEQ ID NO:1 (N27), 827-845 of SEQ ID NO:1 (N28), 869-887 of SEQ ID NO:1 (C2), 883-901 of SEQ ID NO:1 (C3), 911-929 of SEQ ID NO:1 (C5), 925-943 of SEQ ID NO:l (C6), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1065-1083 of SEQ ID NO:1 (C16), 1079-1097 of SEQ ID NO:1 (C17), 1107-1125 of SEQ ID NO:1 (C19), 1121-1139 of SEQ ID NO:l (C20), 1135-1153 of SEQ ID NO:1 (C21), 1149-1167 of SEQ ID NO:1 (C22), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28), 1247-1265 of SEQ ID NO:1 (C29), 1261-1279 of SEQ ID NO:1 (C30), or 1275-1296 of SEQ ID NO:1 (C31). In an aspect of this embodiment, such a BoNT/A peptide is selected from one of the following amino acid sequences: 463-481 of SEQ ID NO:1 (N2), 519-537 of SEQ ID NO:1 (N6), 533-551 of SEQ ID NO:1 (N7), 547-565 of SEQ ID NO:1 (N8), 561-579 of SEQ ID NO:1 (N9), 589-607 of SEQ ID NO:1 (N11), 659-677 of SEQ ID NO:1 (N16), 743-761 of SEQ ID NO:1 (N22), 785-803 of SEQ ID NO:1 (N25), 813-831 of SEQ ID NO:1 (N27), 827-845 of SEQ ID NO:1 (N28), 869-887 of SEQ ID NO:1 (C2), 883-901 of SEQ ID NO:1 (C3), 911-929 of SEQ ID NO:1 (C6), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1121-1139 of SEQ ID NO:1 (C20), 1135-1153 of SEQ ID NO:1 (C21), 1177-1195 of SEQ ID NO:1 (C24), 1261-1279 of SEQ ID NO:1 (C30) or 1275-1296 of SEQ ID NO:1 (C31). In another aspect of this embodiment, such a BoNT/A peptide is selected from one of the following amino acid sequences: 463-481 of SEQ ID NO:1 (N2), 533-551 of SEQ ID NO:1 (N7), 547-565 of SEQ ID NO:1 (N8), 561-579 of SEQ ID NO:1 (N9), 659-677 of SEQ ID NO:1 (N16), 743-761 of SEQ ID NO:1 (N22), 785-803 of SEQ ID NO:1 (N25), 813-831 of SEQ ID NO:1 (N27), 883-901 of SEQ ID NO:1 (C3), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1121-1139 of SEQ ID NO:1 (C20) or 1177-1195 of SEQ ID NO:1 (C24) or 1275-1296 of SEQ ID NO: 1 (C31). In another aspect of this embodiment, such a BoNT/A peptide is selected from one of the following amino acid sequences: 533-551 of SEQ ID NO: 1 (N7), 547-565 of SEQ ID NO: 1 (N8), 743-761 of SEQ ID NO: 1 (N22), 785-803 of SEQ ID NO: 1 (N25), 813-831 of SEQ ID NO: 1 (N27), 995-1013 of SEQ ID NO: 1 (C11); 1051-1069 of SEQ ID NO: 1 (C15), 1177-1195 of SEQ ID NO: 1 (C24), or 1275-1296 of SEQ ID NO: 1 (C31).

In yet another aspect of this embodiment, a BoNT/A peptide useful in a method disclosed in the present specification for preventing or reducing development of a BoNT-specific immune response is selected from one of the following BoNT/A amino acid sequences: 463-481 of SEQ ID NO:1 (N2), 505-523 of SEQ ID NO:1 (N5), 519-537 of SEQ ID NO:1 (N6), 533-551 of SEQ ID NO:1 (N7), 603-621 of SEQ ID NO:1 (N12), 645-663 of SEQ ID NO:1 (N15), 659-677 of SEQ ID NO:1 (N16), 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1079-1097 of SEQ ID NO:1 (C17), 1107-1125 of SEQ ID NO:1 (C19), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28) or 1275-1296 of SEQ ID NO:1 (C31). In yet another aspect of this embodiment, such a BoNT/A peptide is selected from one of the following amino acid sequences: 533-551 of SEQ ID NO:1 (N7), 659-677 of SEQ ID NO:1 (N16), 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1107-1125 of SEQ ID NO:1 (C19), 1163-1181 of SEQ ID NO:1 (C23), 1191-1209 of SEQ ID NO:1 (C25), 1233-1251 of SEQ ID NO:1 (C28) or 1275-1296 of SEQ ID NO:1 (C31). In yet another aspect of this embodiment, such a BoNT/A peptide is selected from one of the following amino acid sequences: 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1079-1097 of SEQ ID NO:l (C17), 1107-1125 of SEQ ID NO:1 (C19), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28) or 1275-1296 of SEQ ID NO:1 (C31). In yet another aspect of this embodiment, such a BoNT/A peptide is selected from one of the following amino acid sequences: 729-747 of SEQ ID NO:1 (N21) 799-817 of SEQ ID NO:1 (N26), 1065-1083 of SEQ ID NO:1 (C16), 1163-1181 of SEQ ID NO:1 (C23) or 1275-1296 of SEQ ID NO:1 (C31).

In yet another embodiment of the present invention, a BoNT/A peptide useful in a method disclosed in the present specification for preventing or reducing development of a BoNT-specific immune response has a length of at most 60 amino acids and consisting of at least 5 contiguous amino acids selected from one of the following BoNT/A amino acid sequences: 449-467 of SEQ ID NO:1(N1), 463-481 of SEQ ID NO:1 (N2), 491-509 of SEQ ID NO:1 (N4), 505-523 of SEQ ID NO:1 (N5), 519-537 of SEQ ID NO:1 (N6), 533-551 of SEQ ID NO:1 (N7), 547-565 of SEQ ID NO:1 (N8), 561-579 of SEQ ID NO:1 (N9), 575-593 of SEQ ID NO:1 (N10), 589-607 of SEQ ID NO:1 (N11), 631-649 of SEQ ID NO:1 (N14), 645-663 of SEQ ID NO:1 (N15), 659-677 of SEQ ID NO:1 (N16), 673-691 of SEQ ID NO:1 (N17), 701-719 of SEQ ID NO:1 (N19), 715-733 of SEQ ID NO:1 (N20), 729-747 of SEQ ID NO:1 (N21), 743-761 of SEQ ID NO:1 (N22), 757-775 of SEQ ID NO:1 (N23), 771-789 of SEQ ID NO:1 (N24), 785-803 of SEQ ID NO:1 (N25), 799-817 of SEQ ID NO:1 (N26), 813-831 of SEQ ID NO:1 (N27), 827-845 of SEQ ID NO:1 (N28), 869-887 of SEQ ID NO:1 (C2), 883-901 of SEQ ID NO:1 (C3), 911-929 of SEQ ID NO:1 (C5), 925-943 of SEQ ID NO:1 (C6), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1065-1083 of SEQ ID NO:1 (C16), 1079-1097 of SEQ ID NO:1 (C17), 1107-1125 of SEQ ID NO:1 (C19), 1121-1139 of SEQ ID NO:1 (C20), 1135-1153 of SEQ ID NO:1 (C21), 1149-1167 of SEQ ID NO:1 (C22), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28), 1247-1265 of SEQ ID NO:1 (C29), 1261-1279 of SEQ ID NO:1 (C30), or 1275-1296 of SEQ ID NO:1 (C31), or a conservative variant thereof. In an aspect of this embodiment, such a BoNT/A peptide is selected from one of the following amino acid sequences: 463-481 of SEQ ID NO:1 (N2), 519-537 of SEQ ID NO:1 (N6), 533-551 of SEQ ID NO:1 (N7), 547-565 of SEQ ID NO:1 (N8), 561-579 of SEQ ID NO:1 (N9), 589-607 of SEQ ID NO:1 (N11), 659-677 of SEQ ID NO:1 (N16), 743-761 of SEQ ID NO:1 (N22), 785-803 of SEQ ID NO:1 (N25), 813-831 of SEQ ID NO:1 (N27), 827-845 of SEQ ID NO:1 (N28), 869-887 of SEQ ID NO:1 (C2), 883-901 of SEQ ID NO:1 (C3), 911-929 of SEQ ID NO:1 (C6), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1121-1139 of SEQ ID NO:1 (C20), 1135-1153 of SEQ ID NO:1 (C21), 1177-1195 of SEQ ID NO:1 (C24), 1261-1279 of SEQ ID NO:1 (C30) or 1275-1296 of SEQ ID NO:1 (C31), or a conservative variant thereof. In another aspect of this embodiment, such a BoNT/A peptide is selected from one of the following amino acid sequences: 463-481 of SEQ ID NO:1 (N2), 533-551 of SEQ ID NO:1 (N7), 547-565 of SEQ ID NO:1 (N8), 561-579 of SEQ ID NO:1 (N9), 659-677 of SEQ ID NO:1 (N16), 743-761 of SEQ ID NO:1 (N22), 785-803 of SEQ ID NO:1 (N25), 813-831 of SEQ ID NO:1 (N27), 883-901 of SEQ ID NO:1 (C3), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1121-1139 of SEQ ID NO:1 (C20) or 1177-1195 of SEQ ID NO:1 (C24) or 1275-1296 of SEQ ID NO: 1 (C31), or a conservative variant thereof. In another aspect of this embodiment, such a BoNT/A peptide is selected from one of the following amino acid sequences: 533-551 of SEQ ID NO: 1 (N7), 547-565 of SEQ ID NO: 1 (N8), 743-761 of SEQ ID NO: 1 (N22), 785-803 of SEQ ID NO: 1 (N25), 813-831 of SEQ ID NO: 1 (N27), 995-1013 of SEQ ID NO: 1 (C11); 1051-1069 of SEQ ID NO: 1 (C15), 1177-1195 of SEQ ID NO: 1 (C24), or 1275-1296 of SEQ ID NO: 1 (C31), or a conservative variant thereof.

In yet another aspect of this embodiment, a BoNT/A peptide useful in a method disclosed in the present specification for preventing or reducing development of a BoNT-specific immune response has a length of at most 60 amino acids and consisting of at least 5 contiguous amino acids selected from one of the following BoNT/A amino acid sequences: 463-481 of SEQ ID NO:1 (N2), 505-523 of SEQ ID NO:1 (N5), 519-537 of SEQ ID NO:1 (N6), 533-551 of SEQ ID NO:1 (N7), 603-621 of SEQ ID NO:1 (N12), 645-663 of SEQ ID NO:1 (N15), 659-677 of SEQ ID NO:1 (N16), 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1079-1097 of SEQ ID NO:1 (C17), 1107-1125 of SEQ ID NO:1 (C19), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28) or 1275-1296 of SEQ ID NO:1 (C31), or a conservative variant thereof. In yet another aspect of this embodiment, such a BoNT/A peptide is selected from one of the following amino acid sequences: 533-551 of SEQ ID NO:1 (N7), 659-677 of SEQ ID NO:1 (N16), 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1107-1125 of SEQ ID NO:1 (C19), 1163-1181 of SEQ ID NO:1 (C23), 1191-1209 of SEQ ID NO:1 (C25), 1233-1251 of SEQ ID NO:1 (C28) or 1275-1296 of SEQ ID NO:1 (C31), or a conservative variant thereof. In yet another aspect of this embodiment, such a BoNT/A peptide is selected from one of the following amino acid sequences: 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1079-1097 of SEQ ID NO:1 (C17), 1107-1125 of SEQ ID NO:1 (C19), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28) or 1275-1296 of SEQ ID NO:1 (C31), or a conservative variant thereof. In yet another aspect of this embodiment, such a BoNT/A peptide is selected from one of the following amino acid sequences: 729-747 of SEQ ID NO:1 (N21) 799-817 of SEQ ID NO:1 (N26), 1065-1083 of SEQ ID NO:1 (C16), 1163-1181 of SEQ ID NO:1 (C23) or 1275-1296 of SEQ ID NO:1 (C31), or a conservative variant thereof.

In yet another embodiment of the present invention, a BoNT/A peptide useful in a method disclosed in the present specification for preventing or reducing development of a BoNT-specific immune response has a length of at most 60 amino acids and consisting of at least 5 contiguous amino acids selected from one of the following BoNT/A amino acid sequences: 449-467 of SEQ ID NO:l (N1), 463-481 of SEQ ID NO:l (N2), 491-509 of SEQ ID NO:1 (N4), 505-523 of SEQ ID NO:1 (N5), 519-537 of SEQ ID NO:1 (N6), 533-551 of SEQ ID NO:1 (N7), 547-565 of SEQ ID NO:1 (N8), 561-579 of SEQ ID NO:1 (N9), 575-593 of SEQ ID NO:1 (N10), 589-607 of SEQ ID NO:1 (N11), 631-649 of SEQ ID NO:1 (N14), 645-663 of SEQ ID NO:1 (N15), 659-677 of SEQ ID NO:1 (N16), 673-691 of SEQ ID NO:1 (N17), 701-719 of SEQ ID NO:1 (N19), 715-733 of SEQ ID NO:1 (N20), 729-747 of SEQ ID NO:1 (N21), 743-761 of SEQ ID NO:1 (N22), 757-775 of SEQ ID NO:1 (N23), 771-789 of SEQ ID NO:1 (N24), 785-803 of SEQ ID NO:1 (N25), 799-817 of SEQ ID NO:1 (N26), 813-831 of SEQ ID NO:1 (N27), 827-845 of SEQ ID NO:1 (N28), 869-887 of SEQ ID NO:1 (C2), 883-901 of SEQ ID NO:1 (C3), 911-929 of SEQ ID NO:1 (C5), 925-943 of SEQ ID NO:l (C6), 939-957 of SEQ ID NO:1 (C7), 967-985 of SEQ ID NO:1 (C9), 981-999 of SEQ ID NO:1 (Co), 995-1013 of SEQ ID NO:1 (C11), 1051-1069 of SEQ ID NO:1 (C15), 1065-1083 of SEQ ID NO:1 (C16), 1079-1097 of SEQ ID NO:1 (C17), 1107-1125 of SEQ ID NO:1 (C19), 1121-1139 of SEQ ID NO:1 (C20), 1135-1153 of SEQ ID NO:1 (C21), 1149-1167 of SEQ ID NO:1 (C22), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28), 1247-1265 of SEQ ID NO:1 (C29), 1261-1279 of SEQ ID NO:1 (C30), or 1275-1296 of SEQ ID NO:1 (C31), or a non-conservative variant thereof. In an aspect of this embodiment, such a BoNT/A peptide is selected from one of the following amino acid sequences: 463-481 of SEQ ID NO:1 (N2), 519-537 of SEQ ID NO:1 (N6), 533-551 of SEQ ID NO:1 (N7), 547-565 of SEQ ID NO:1 (N8), 561-579 of SEQ ID NO:1 (N9), 589-607 of SEQ ID NO:1 (N11), 659-677 of SEQ ID NO:1 (N16), 743-761 of SEQ ID NO:1 (N22), 785-803 of SEQ ID NO:1 (N25), 813-831 of SEQ ID NO:1 (N27), 827-845 of SEQ ID NO:1 (N28), 869-887 of SEQ ID NO:1 (C2), 883-901 of SEQ ID NO:1 (C3), 911-929 of SEQ ID NO:1 (C6), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1121-1139 of SEQ ID NO:1 (C20), 1135-1153 of SEQ ID NO:1 (C21), 1177-1195 of SEQ ID NO:1 (C24), 1261-1279 of SEQ ID NO:1 (C30) or 1275-1296 of SEQ ID NO:1 (C31), or a non-conservative variant thereof. In another aspect of this embodiment, such a BoNT/A peptide is selected from one of the following amino acid sequences: 463-481 of SEQ ID NO:1 (N2), 533-551 of SEQ ID NO:1 (N7), 547-565 of SEQ ID NO:1 (N8), 561-579 of SEQ ID NO:1 (N9), 659-677 of SEQ ID NO:1 (N16), 743-761 of SEQ ID NO:1 (N22), 785-803 of SEQ ID NO:1 (N25), 813-831 of SEQ ID NO:1 (N27), 883-901 of SEQ ID NO:1 (C3), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1121-1139 of SEQ ID NO:1 (C20) or 1177-1195 of SEQ ID NO:1 (C24) or 1275-1296 of SEQ ID NO: 1 (C31), or a non-conservative variant thereof. In another aspect of this embodiment, such a BoNT/A peptide is selected from one of the following amino acid sequences: 533-551 of SEQ ID NO: 1 (N7), 547-565 of SEQ ID NO: 1 (N8), 743-761 of SEQ ID NO: 1 (N22), 785-803 of SEQ ID NO: 1 (N25), 813-831 of SEQ ID NO: 1 (N27), 995-1013 of SEQ ID NO: 1 (C11); 1051-1069 of SEQ ID NO: 1 (C15), 1177-1195 of SEQ ID NO: 1 (C24), or 1275-1296 of SEQ ID NO: 1 (C31), or a non-conservative variant thereof.

In yet another aspect of this embodiment, a BoNT/A peptide useful in a method disclosed in the present specification for preventing or reducing development of a BoNT-specific immune response has a length of at most 60 amino acids and consisting of at least 5 contiguous amino acids selected from one of the following BoNT/A amino acid sequences: 463-481 of SEQ ID NO:1 (N2), 505-523 of SEQ ID NO:1 (N5), 519-537 of SEQ ID NO:1 (N6), 533-551 of SEQ ID NO:1 (N7), 603-621 of SEQ ID NO:1 (N12), 645-663 of SEQ ID NO:1 (N15), 659-677 of SEQ ID NO:1 (N16), 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1079-1097 of SEQ ID NO:1 (C17), 1107-1125 of SEQ ID NO:1 (C19), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28) or 1275-1296 of SEQ ID NO:1 (C31), or a non-conservative variant thereof. In yet another aspect of this embodiment, such a BoNT/A peptide is selected from one of the following amino acid sequences: 533-551 of SEQ ID NO:1 (N7), 659-677 of SEQ ID NO:1 (N16), 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1107-1125 of SEQ ID NO:1 (C19), 1163-1181 of SEQ ID NO:1 (C23), 1191-1209 of SEQ ID NO:1 (C25), 1233-1251 of SEQ ID NO:1 (C28) or 1275-1296 of SEQ ID NO:1 (C31), or a non-conservative variant thereof. In yet another aspect of this embodiment, such a BoNT/A peptide is selected from one of the following amino acid sequences: 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1079-1097 of SEQ ID NO:1 (C17), 1107-1125 of SEQ ID NO:1 (C19), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28) or 1275-1296 of SEQ ID NO:1 (C31), or a non-conservative variant thereof. In yet another aspect of this embodiment, such a BoNT/A peptide is selected from one of the following amino acid sequences: 729-747 of SEQ ID NO:1 (N21) 799-817 of SEQ ID NO:1 (N26), 1065-1083 of SEQ ID NO:1 (C16), 1163-1181 of SEQ ID NO:1 (C23) or 1275-1296 of SEQ ID NO:1 (C31), or a non-conservative variant thereof.

In yet another embodiment of the present invention, a BoNT/A peptide useful in a method disclosed in the present specification for preventing or reducing development of a BoNT-specific immune response has a length of at most 60 amino acids and consisting of at least 5 contiguous amino acids selected from one of the following BoNT/A amino acid sequences: 449-467 of SEQ ID NO:1(N1), 463-481 of SEQ ID NO:1 (N2), 491-509 of SEQ ID NO:1 (N4), 505-523 of SEQ ID NO:1 (N5), 519-537 of SEQ ID NO:1 (N6), 533-551 of SEQ ID NO:1 (N7), 547-565 of SEQ ID NO:1 (N8), 561-579 of SEQ ID NO:1 (N9), 575-593 of SEQ ID NO:1 (N10), 589-607 of SEQ ID NO:1 (N11), 631-649 of SEQ ID NO:1 (N14), 645-663 of SEQ ID NO:1 (N15), 659-677 of SEQ ID NO:1 (N16), 673-691 of SEQ ID NO:1 (N17), 701-719 of SEQ ID NO:1 (N19), 715-733 of SEQ ID NO:1 (N20), 729-747 of SEQ ID NO:1 (N21), 743-761 of SEQ ID NO:1 (N22), 757-775 of SEQ ID NO:1 (N23), 771-789 of SEQ ID NO:1 (N24), 785-803 of SEQ ID NO:1 (N25), 799-817 of SEQ ID NO:1 (N26), 813-831 of SEQ ID NO:1 (N27), 827-845 of SEQ ID NO:1 (N28), 869-887 of SEQ ID NO:1 (C2), 883-901 of SEQ ID NO:1 (C3), 911-929 of SEQ ID NO:1 (C5), 925-943 of SEQ ID NO:1 (C6), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1065-1083 of SEQ ID NO:1 (C16), 1079-1097 of SEQ ID NO:1 (C17), 1107-1125 of SEQ ID NO:1 (C19), 1121-1139 of SEQ ID NO:1 (C20), 1135-1153 of SEQ ID NO:1 (C21), 1149-1167 of SEQ ID NO:1 (C22), 1163-1181 of SEQ ID NO:1 (C23), 1177-1195 of SEQ ID NO:1 (C24), 1191-1209 of SEQ ID NO:l (C25), 1233-1251 of SEQ ID NO:1 (C28), 1247-1265 of SEQ ID NO:1 (C29), 1261-1279 of SEQ ID NO:1 (C30), or 1275-1296 of SEQ ID NO:1 (C31), or an immunoreactive fragment thereof, with the proviso that the BoNT/A peptide is not SEQ ID NO:2. In an aspect of this embodiment, such a BoNT/A peptide is selected from one of the following amino acid sequences: 463-481 of SEQ ID NO:1 (N2), 519-537 of SEQ ID NO:1 (N6), 533-551 of SEQ ID NO:1 (N7), 547-565 of SEQ ID NO:1 (N8), 561-579 of SEQ ID NO:1 (N9), 589-607 of SEQ ID NO:1 (N11), 659-677 of SEQ ID NO:1 (N16), 743-761 of SEQ ID NO:1 (N22), 785-803 of SEQ ID NO:1 (N25), 813-831 of SEQ ID NO:1 (N27), 827-845 of SEQ ID NO:1 (N28), 869-887 of SEQ ID NO:1 (C2), 883-901 of SEQ ID NO:1 (C3), 911-929 of SEQ ID NO:1 (C6), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1121-1139 of SEQ ID NO:1 (C20), 1135-1153 of SEQ ID NO:1 (C21), 1177-1195 of SEQ ID NO:1 (C24), 1261-1279 of SEQ ID NO:1 (C30) or 1275-1296 of SEQ ID NO:1 (C31), or an immunoreactive fragment thereof, with the proviso that the BoNT/A peptide is not SEQ ID NO:2. In another aspect of this embodiment, such a BoNT/A peptide is selected from one of the following amino acid sequences: 463-481 of SEQ ID NO:1 (N2), 533-551 of SEQ ID NO:1 (N7), 547-565 of SEQ ID NO:1 (N8), 561-579 of SEQ ID NO:1 (N9), 659-677 of SEQ ID NO:1 (N16), 743-761 of SEQ ID NO:1 (N22), 785-803 of SEQ ID NO:l (N25), 813-831 of SEQ ID NO:1 (N27), 883-901 of SEQ ID NO:1 (C3), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1121-1139 of SEQ ID NO:1 (C20) or 1177-1195 of SEQ ID NO:1 (C24) or 1275-1296 of SEQ ID NO: 1 (C31), or an immunoreactive fragment thereof, with the proviso that the BoNT/A peptide is not SEQ ID NO:2. In another aspect of this embodiment, such a BoNT/A peptide is selected from one of the following amino acid sequences: 533-551 of SEQ ID NO: 1 (N7), 547-565 of SEQ ID NO: 1 (N8), 743-761 of SEQ ID NO: 1 (N22), 785-803 of SEQ ID NO: 1 (N25), 813-831 of SEQ ID NO: 1 (N27), 995-1013 of SEQ ID NO: 1 (C11); 1051-1069 of SEQ ID NO: 1 (C15), 1177-1195 of SEQ ID NO: 1 (C24), or 1275-1296 of SEQ ID NO: 1 (C31), or an immunoreactive fragment thereof, with the proviso that the BoNT/A peptide is not SEQ ID NO:2.

In yet another aspect of this embodiment, a BoNT/A peptide useful in a method disclosed in the present specification for preventing or reducing development of a BoNT-specific immune response has a length of at most 60 amino acids and consisting of at least 5 contiguous amino acids selected from one of the following BoNT/A amino acid sequences: 463-481 of SEQ ID NO:1 (N2), 505-523 of SEQ ID NO:1 (N5), 519-537 of SEQ ID NO:1 (N6), 533-551 of SEQ ID NO:1 (N7), 603-621 of SEQ ID NO:1 (N12), 645-663 of SEQ ID NO:1 (N15), 659-677 of SEQ ID NO:1 (N16), 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1079-1097 of SEQ ID NO:1 (C17), 1107-1125 of SEQ ID NO:1 (C19), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28) or 1275-1296 of SEQ ID NO:1 (C31), or an immunoreactive fragment thereof, with the proviso that the BoNT/A peptide is not SEQ ID NO:2. In yet another aspect of this embodiment, such a BoNT/A peptide is selected from one of the following amino acid sequences: 533-551 of SEQ ID NO:1 (N7), 659-677 of SEQ ID NO:1 (N16), 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1107-1125 of SEQ ID NO:1 (C19), 1163-1181 of SEQ ID NO:1 (C23), 1191-1209 of SEQ ID NO:l (C25), 1233-1251 of SEQ ID NO:1 (C28) or 1275-1296 of SEQ ID NO:1 (C31), or an immunoreactive fragment thereof, with the proviso that the BoNT/A peptide is not SEQ ID NO:2. In yet another aspect of this embodiment, such a BoNT/A peptide is selected from one of the following amino acid sequences: 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1079-1097 of SEQ ID NO:1 (C17), 1107-1125 of SEQ ID NO:1 (C19), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28) or 1275-1296 of SEQ ID NO:1 (C31), or an immunoreactive fragment thereof, with the proviso that the BoNT/A peptide is not SEQ ID NO:2. In yet another aspect of this embodiment, such a BoNT/A peptide is selected from one of the following amino acid sequences: 729-747 of SEQ ID NO:1 (N21) 799-817 of SEQ ID NO:1 (N26), 1065-1083 of SEQ ID NO:1 (C16), 1163-1181 of SEQ ID NO:1 (C23) or 1275-1296 of SEQ ID NO:l (C31), or an immunoreactive fragment thereof, with the proviso that the BoNT/A peptide is not SEQ ID NO:2.

In is envisioned that a BoNT/A peptide useful in a method disclosed in the present specification for preventing or reducing development of a BoNT-specific immune response can have any of a variety of lengths from at least 5 amino acids to at most 60 amino acids. Therefore, aspects of this embodiment may include a BoNT/A peptide with at least, e.g., five amino acids, six amino acids, seven amino acids, eight amino acids, nine amino acids, ten amino acids, 11 amino acids, 12 amino acids, 13 amino acids, 14 amino acids, 15 amino acids, 16 amino acids, 17 amino acids, 18 amino acids, 19 amino acids, 20 amino acids, 25 amino acids, 30 amino acids, 35 amino acids, 40 amino acids, 45 amino acids, 50 amino acids, 55 amino acids or 60 amino acids. Other aspects of this embodiment may include a BoNT/A peptide with at least, e.g., five amino acids of SEQ ID NO:1, six amino acids of SEQ ID NO:1, seven amino acids of SEQ ID NO:1, eight amino acids of SEQ ID NO:1, nine amino acids of SEQ ID NO:1, ten amino acids of SEQ ID NO:1, 11 amino acids of SEQ ID NO:1, 12 amino acids of SEQ ID NO:1, 13 amino acids of SEQ ID NO:1, 14 amino acids of SEQ ID NO:1, 15 amino acids of SEQ ID NO:1, 16 amino acids of SEQ ID NO:1, 17 amino acids of SEQ ID NO:1, 18 amino acids of SEQ ID NO:1, 19 amino acids of SEQ ID NO:1, 20 amino acids of SEQ ID NO:1, 25 amino acids of SEQ ID NO:1, 30 amino acids of SEQ ID NO:1, 35 amino acids of SEQ ID NO:1, 40 amino acids of SEQ ID NO:1, 45 amino acids of SEQ ID NO:1, 50 amino acids of SEQ ID NO:1, 55 amino acids or 60 amino acids of SEQ ID NO:1. In further embodiments, such a BoNT/A peptide of the invention may include a BoNT/A peptide with at least, e.g., five amino acids, six amino acids, seven amino acids, eight amino acids, nine amino acids, ten amino acids, 11 amino acids, 12 amino acids, 13 amino acids, 14 amino acids, 15 amino acids, 16 amino acids, 17 amino acids, 18 amino acids, 19 amino acids, 20 amino acids, 25 amino acids, 30 amino acids, 35 amino acids, 40 amino acids, 45 amino acids, 50 amino acids, 55 amino acids or 60 amino acids and consist of at least 5 contiguous amino acids selected from one of the following BoNT/A amino acid sequences: 449-467 of SEQ ID NO:1(N1), 463-481 of SEQ ID NO:1 (N2), 491-509 of SEQ ID NO:1 (N4), 505-523 of SEQ ID NO:1 (N5), 519-537 of SEQ ID NO:1 (N6), 533-551 of SEQ ID NO:1 (N7), 547-565 of SEQ ID NO:1 (N8), 561-579 of SEQ ID NO:1 (N9), 575-593 of SEQ ID NO:1 (N10), 589-607 of SEQ ID NO:1 (N11), 631-649 of SEQ ID NO:1 (N14), 645-663 of SEQ ID NO:1 (N15), 659-677 of SEQ ID NO:1 (N16), 673-691 of SEQ ID NO:1 (N17), 701-719 of SEQ ID NO:1 (N19), 715-733 of SEQ ID NO:1 (N20), 729-747 of SEQ ID NO:1 (N21), 743-761 of SEQ ID NO:1 (N22), 757-775 of SEQ ID NO:1 (N23), 771-789 of SEQ ID NO:1 (N24), 785-803 of SEQ ID NO:1 (N25), 799-817 of SEQ ID NO:1 (N26), 813-831 of SEQ ID NO:1 (N27), 827-845 of SEQ ID NO:1 (N28), 869-887 of SEQ ID NO:1 (C2), 883-901 of SEQ ID NO:1 (C3), 911-929 of SEQ ID NO:1 (C5), 925-943 of SEQ ID NO:1 (C6), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1065-1083 of SEQ ID NO:1 (C16), 1079-1097 of SEQ ID NO:1 (C17), 1107-1125 of SEQ ID NO:1 (C19), 1121-1139 of SEQ ID NO:1 (C20), 1135-1153 of SEQ ID NO:1 (C21), 1149-1167 of SEQ ID NO:1 (C22), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28), 1247-1265 of SEQ ID NO:1 (C29), 1261-1279 of SEQ ID NO:1 (C30) or 1275-1296 of SEQ ID NO:l (C31), or a conservative variant, a non-conservative variant, or immunoreactive fragment thereof, with the proviso that the BoNT/A peptide is not SEQ ID NO:2.

In another embodiment of the present invention, a BoNT/A peptide useful in a method disclosed in the present specification for preventing or reducing development of a BoNT-specific immune response can comprise one BoNT/A peptide disclosed in the present specification. In another embodiment of the present invention, a BoNT/A peptide useful in a method disclosed in the present specification for preventing or reducing development of a BoNT-specific immune response can comprise a plurality of BoNT/A peptides disclosed in the present specification. Thus, aspects of this embodiment can include one or more BoNT/A peptides, two or more BoNT/A peptides, three or more BoNT/A peptides, four or more BoNT/A peptides, five or more BoNT/A peptides, six or more BoNT/A peptides, seven or more BoNT/A peptides, eight or more BoNT/A peptides, nine or more BoNT/A peptides, ten or more BoNT/A peptides, 15 or more BoNT/A peptides, 20 or more BoNT/A peptides, 25 or more BoNT/A peptides or 30 or more BoNT/A peptides. In other aspects of this embodiment can include one or more BoNT/A conservative variants, two or more BoNT/A conservative variants, three or more BoNT/A conservative variants, four or more BoNT/A conservative variants, five or more BoNT/A conservative variants, six or more BoNT/A conservative variants, seven or more BoNT/A conservative variants, eight or more BoNT/A conservative variants, nine or more BoNT/A conservative variants, ten or more BoNT/A conservative variants, 15 or more BoNT/A conservative variants, 20 or more BoNT/A conservative variants, 25 or more BoNT/A conservative variants or 30 or more BoNT/A conservative variants. In further aspects of this embodiment can include one or more BoNT/A non-conservative variants, two or more BoNT/A non-conservative variants, three or more BoNT/A non-conservative variants, four or more BoNT/A non-conservative variants, five or more BoNT/A non-conservative variants, six or more BoNT/A non-conservative variants, seven or more BoNT/A non-conservative variants, eight or more BoNT/A non-conservative variants, nine or more BoNT/A non-conservative variants, ten or more BoNT/A non-conservative variants, 15 or more BoNT/A non-conservative variants, 20 or more BoNT/A non-conservative variants, 25 or more BoNT/A non-conservative variants or 30 or more BoNT/A non-conservative variants. In still other aspects of this embodiment can include one or more BoNT/A immunoreactive fragments, two or more BoNT/A immunoreactive fragments, three or more BoNT/A immunoreactive fragments, four or more BoNT/A immunoreactive fragments, five or more BoNT/A immunoreactive fragments, six or more BoNT/A immunoreactive fragments, seven or more BoNT/A immunoreactive fragments, eight or more BoNT/A immunoreactive fragments, nine or more BoNT/A peptides, ten or more BoNT/A immunoreactive fragments, 15 or more BoNT/A immunoreactive fragments, 20 or more BoNT/A immunoreactive fragments, 25 or more BoNT/A immunoreactive fragments or 30 or more BoNT/A immunoreactive fragments. BoNT/A peptides disclosed in the present specification useful for preventing or reducing development of a BoNT-specific immune response can be selected, for example, depending on immunological factors, such as potency of the peptide in inducing an immune response, and technical factors, such as chemical synthesis yields. It is also understood that the two or more BoNT/A peptides can be provided separately or as part of a compound molecule such as a chimeric peptide or heterologous protein.

In an aspect of this embodiment, a method of preventing or reducing development of a BoNT-specific immune response uses two or more immunoreactive BoNT/A peptides selected from the following amino acid sequences: 533-551 of SEQ ID NO:1 (N7), 547-565 of SEQ ID NO:1 (N8), 743-761 of SEQ ID NO:1 (N22), 785-803 of SEQ ID NO:1 (N25), 813-831 of SEQ ID NO:1 (N27), 995-1013 of SEQ ID NO:1 (C11); 1051-1069 of SEQ ID NO:1 (C15), 1177-1195 of SEQ ID NO:1 (C24), and 1275-1296 of SEQ ID NO:1 (C31), or a conservative variant, a non-conservative variant or an immunoreactive fragment thereof, with the proviso that the BoNT/A peptide is not SEQ ID NO:2. In another aspect of this embodiment, one of the selected amino acid sequence is 533-551 of SEQ ID NO:1 (N8) or a conservative variant, a non-conservative variant or an immunoreactive fragment thereof, with the proviso that the BoNT/A peptide is not SEQ ID NO:2. In yet another aspect of this embodiment, the following two amino acid sequences are selected: 533-551 of SEQ ID NO:1 (N8) and 981-999 of SEQ ID NO:1 (C10), or a conservative variant, a non-conservative variant or an immunoreactive fragment thereof, with the proviso that the BoNT/A peptide is not SEQ ID NO:2. In yet another aspect of this embodiment, the following three amino acid sequences are selected: 533-551 of SEQ ID NO:1 (N8), 981-999 of SEQ ID NO:1 (C10) and 1051-1069 of SEQ ID NO:1 (C15), or a conservative variant, a non-conservative variant or an immunoreactive fragment thereof, with the proviso that the BoNT/A peptide is not SEQ ID NO:2. In a further aspect of this embodiment, one of the amino acid sequences selected is 785-803 of SEQ ID NO:1 (N25) or a conservative variant, a non-conservative variant or an immunoreactive fragment thereof, with the proviso that the BoNT/A peptide is not SEQ ID NO:2. In a still further aspect of this embodiment, the following two amino acid sequences are selected: 785-803 of SEQ ID NO:1 (N25) and 981-999 of SEQ ID NO:1 (C10), or a conservative variant, a non-conservative variant or an immunoreactive fragment thereof, with the proviso that the BoNT/A peptide is not SEQ ID NO:2. In a still further aspect of this embodiment, the following three amino acid sequences are selected: 785-803 of SEQ ID NO:1 (N25), 981-999 of SEQ ID NO:1 (C10) and 1051-1069 of SEQ ID NO:1 (C15), or a conservative variant, a non-conservative variant or an immunoreactive fragment thereof, with the proviso that the BoNT/A peptide is not SEQ ID NO:2. In an additional aspect of this embodiment, one of the amino acid sequences selected is 813-831 of SEQ ID NO:1 (N27) or a conservative variant, a non-conservative variant or an immunoreactive fragment thereof, with the proviso that the BoNT/A peptide is not SEQ ID NO:2. In another additional aspect of this embodiment, the following two amino acid sequences are selected: 813-831 of SEQ ID NO:1 (N27) and 981-999 of SEQ ID NO:1 (C10), or a conservative variant, a non-conservative variant or an immunoreactive fragment thereof, with the proviso that the BoNT/A peptide is not SEQ ID NO:2. In another additional aspect of this embodiment, the following three amino acid sequences are selected: 813-831 of SEQ ID NO:1 (N27), 981-999 of SEQ ID NO:1 (C10) and 1051-1069 of SEQ ID NO:1 (C15), or a conservative variant, a non-conservative variant or an immunoreactive fragment thereof, with the proviso that the BoNT/A peptide is not SEQ ID NO:2.

In an aspect of this embodiment, a method of preventing or reducing development of a BoNT-specific immune response uses two or more immunoreactive BoNT/A peptides selected from the following amino acid sequences: 659-677 of SEQ ID NO:1 (N16), 729-747 of SEQ ID NO:1 (N21), 799-817 of SEQ ID NO:1 (N26), 1065-1083 of SEQ ID NO:1 (C16), 1163-1181 of SEQ ID NO:1 (C23), and 1275-1296 of SEQ ID NO:1 (C31), or a conservative variant, a non-conservative variant or an immunoreactive fragment thereof, with the proviso that the BoNT/A peptide is not SEQ ID NO:2. In another aspect of this embodiment, one of the amino acid sequences selected is 1065-1083 of SEQ ID NO:1 (C16) or a conservative variant, a non-conservative variant or an immunoreactive fragment thereof, with the proviso that the BoNT/A peptide is not SEQ ID NO:2. In yet another aspect of this embodiment, the following two amino acid sequences are selected: 1065-1083 of SEQ ID NO:1 (C16) and 1163-1181 of SEQ ID NO:1 (C23), or a conservative variant, a non-conservative variant or an immunoreactive fragment thereof, with the proviso that the BoNT/A peptide is not SEQ ID NO:2. In yet another aspect of this embodiment, the following three amino acid sequences are selected: 1065-1083 of SEQ ID NO:1 (C16), 1163-1181 of SEQ ID NO:1 (C23) and 1275-1296 of SEQ ID NO:1 (C31), or a conservative variant, a non-conservative variant or an immunoreactive fragment thereof, with the proviso that the BoNT/A peptide is not SEQ ID NO:2. In a further aspect of this embodiment, one of the amino acid sequences selected is 799-817 of SEQ ID NO:1 (N26) or a conservative variant, a non-conservative variant or an immunoreactive fragment thereof, with the proviso that the BoNT/A peptide is not SEQ ID NO:2. In a still further aspect of this embodiment, the following two amino acid sequences are selected: 799-817 of SEQ ID NO:1 (N26) and 1065-1083 of SEQ ID NO:1 (C16), or a conservative variant, a non-conservative variant or an immunoreactive fragment thereof, with the proviso that the BoNT/A peptide is not SEQ ID NO:2. In a still further aspect of this embodiment, the following three amino acid sequences are selected: 799-817 of SEQ ID NO:1 (N26), 1065-1083 of SEQ ID NO:1 (C16) and 1163-1181 of SEQ ID NO:1 (C23), or a conservative variant, a non-conservative variant or an immunoreactive fragment thereof, with the proviso that the BoNT/A peptide is not SEQ ID NO:2. In an additional aspect of this embodiment, one of the amino acid sequences selected is 729-747 of SEQ ID NO:1 (N21) or a conservative variant, a non-conservative variant or an immunoreactive fragment thereof, with the proviso that the BoNT/A peptide is not SEQ ID NO:2. In another additional aspect of this embodiment, the following two amino acid sequences are selected: 729-747 of SEQ ID NO:1 (N21) and 1065-1083 of SEQ ID NO:1 (C16), or a conservative variant, a non-conservative variant or an immunoreactive fragment thereof, with the proviso that the BoNT/A peptide is not SEQ ID NO:2. In another additional aspect of this embodiment, the following three amino acid sequences are selected: 729-747 of SEQ ID NO:1 (N21), 1065-1083 of SEQ ID NO:1 (C16) and 1163-1181 of SEQ ID NO:1 (C23), or a conservative variant, a non-conservative variant or an immunoreactive fragment thereof, with the proviso that the BoNT/A peptide is not SEQ ID NO:2.

It is also envisioned that any and all combinations of BoNT/A peptides disclosed in the specification can be useful for preventing or reducing development of a BoNT-specific immune response, including, e.g., BoNT/A peptides of SEQ ID NO:1, BoNT/A conservative variants, BoNT/A non-conservative variants and BoNT/A immunoreactive fragments. Thus, aspects of this embodiment include one or more BoNT/A peptides comprising one or more BoNT/A peptides of SEQ ID NO: 1 and one or more BoNT/A conservative variants; one or more BoNT/A peptides of SEQ ID NO: 1 and one or more BoNT/A non-conservative variants; one or more BoNT/A peptides of SEQ ID NO: 1 and one or more BoNT/A immunoreactive fragments; one or more BoNT/A conservative variants and one or more BoNT/A non-conservative variants; one or more BoNT/A conservative variants and one or more BoNT/A immunoreactive fragments; one or more BoNT/A non-conservative variants and one or more BoNT/A immunoreactive fragments; one or more BoNT/A peptides of SEQ ID NO: 1, one or more BoNT/A conservative variants and one or more BoNT/A non-conservative variants; one or more BoNT/A peptides of SEQ ID NO: 1, one or more BoNT/A conservative variants and one or more BoNT/A immunoreactive fragments; one or more BoNT/A peptides of SEQ ID NO: 1, one or more BoNT/A non-conservative variants and one or more BoNT/A immunoreactive fragments; one or more BoNT/A conservative variants, one or more BoNT/A non-conservative variants and one or more BoNT/A immunoreactive fragments; or one or more BoNT/A peptides of SEQ ID NO: 1, one or more BoNT/A conservative variants, one or more BoNT/A non-conservative variants and one or more BoNT/A immunoreactive fragments.

A tolerogizing agent and BoNT/A peptide can be formulated in a variety of pharmaceutically acceptable media, as described below. An effective dose of a BoNT/A peptide of the invention for inducing tolerance in a human or other mammal will depend upon the particular BoNT/A peptide selected, the tolerogizing agent used, the route administration, and the particular characteristics of the human or other mammal, such as age, weight, general health and the like. An effective dose can be determined in an animal model, such as one of those described hereinabove, prior to administration to humans. Tolerogizing agents and BoNT/A peptides useful in the invention can be administered by a variety of routes to stimulate an immune response. As a non-limiting example, oral tolerance is well-recognized in the art (see, for example, Weiner, Hospital Practice, pp. 53-58 (Sep. 15, 1995). Those skilled in the art can readily determine for a particular tolerogizing composition, a suitable pharmacological composition, an appropriate antigen payload; route of administration; volume of dose; and tolerogizing regimen useful in a particular animal, for example, humans.

As disclosed herein a tolerogizing composition is administered to a human or other mammal to treat a condition characterized by BoNT/A immunoresistance. As used herein, the term “treating,” when used in reference to administering to a human or other mammal an effective amount of a tolerogizing composition, means reducing a symptom of a condition characterized by resistance to a BoNT/A theraphy, or delaying or preventing onset of a symptom of a condition characterized by BoNT/A immunoresistance in the human or other mammal. For example, the term “treating” can mean reducing a symptom of a condition characterized by BoNT/A immunoresistance by at least 30%, 40%, 60%, 70%, 80%, 90% or 100%. The effectiveness of a tolerogizing composition in treating a condition characterized by BoNT/A immunoresistance can be determined by observing one or more clinical symptoms or physiological indicators associated with the condition. An improvement in a condition characterized by BoNT/A immunoresistance also can be indicated by a reduced need for a concurrent therapy. Those of skill in the art will know the appropriate symptoms or indicators associated with specific conditions and will know how to determine if an human or other mammal is a candidate for treatment with a tolerogizing composition disclosed in the present specification. In particular, it is understood that those skilled in the art will be able to determine if a condition if characterized by BoNT/A immunoresistance, for example, by comparison of levels of BoNT/A immunoresistance from the human or other mammal with a normal control cells.

The appropriate effective amount to be administered for a particular application of the methods can be determined by those skilled in the art, using the guidance provided herein. For example, an effective amount can be extrapolated from in vitro and in vivo assays as described herein above. One skilled in the art will recognize that the condition of the patient can be monitored throughout the course of therapy and that the effective amount of a tolerogizing composition that is administered can be adjusted accordingly.

A tolerogizing composition useful in the invention generally is administered in a pharmaceutical acceptable composition. As used herein, the term “pharmaceutically acceptable” refer to any molecular entity or composition that does not produce an adverse, allergic or other untoward or unwanted reaction when administered to a human or other mammal. As used herein, the term “pharmaceutically acceptable composition” refers to a therapeutically effective concentration of an active ingredient. A pharmaceutical composition may be administered to a patient alone, or in combination with other supplementary active ingredients, agents, drugs or hormones. The pharmaceutical compositions may be manufactured using any of a variety of processes, including, without limitation, conventional mixing, dissolving, granulating, dragee-making, levigating, emulsifying, encapsulating, entrapping, and lyophilizing. The pharmaceutical composition can take any of a variety of forms including, without limitation, a sterile solution, suspension, emulsion, lyophilizate, tablet, pill, pellet, capsule, powder, syrup, elixir or any other dosage form suitable for administration.

It is also envisioned that a pharmaceutical composition disclosed in the present specification can optionally include a pharmaceutically acceptable carriers that facilitate processing of an active ingredient into pharmaceutically acceptable compositions. As used herein, the term “pharmacologically acceptable carrier” refers to any carrier that has substantially no long term or permanent detrimental effect when administered and encompasses terms such as “pharmacologically acceptable vehicle, stabilizer, diluent, auxiliary or excipient.” Such a carrier generally is mixed with an active compound, or permitted to dilute or enclose the active compound and can be a solid, semi-solid, or liquid agent. It is understood that the active ingredients can be soluble or can be delivered as a suspension in the desired carrier or diluent. Any of a variety of pharmaceutically acceptable carriers can be used including, without limitation, aqueous media such as, e.g., distilled, deionized water, saline; solvents; dispersion media; coatings; antibacterial and antifungal agents; isotonic and absorption delaying agents; or any other inactive ingredient. Selection of a pharmacologically acceptable carrier can depend on the mode of administration. Except insofar as any pharmacologically acceptable carrier is incompatible with the active ingredient, its use in pharmaceutically acceptable compositions is contemplated. Non-limiting examples of specific uses of such pharmaceutical carriers can be found in PHARMACEUTICAL DOSAGE FORMS AND DRUG DELIVERY SYSTEMS (Howard C. Ansel et al., eds., Lippincott Williams & Wilkins Publishers, 7^{th} ed. 1999); REMINGTON: THE SCIENCE AND PRACTICE OF PHARMACY (Alfonso R. Gennaro ed., Lippincott, Williams & Wilkins, 20^{th} ed. 2000); GOODMAN & GILMAN'S THE PHARMACOLOGICAL BASIS OF THERAPEUTICS (Joel G. Hardman et al., eds., McGraw-Hill Professional, 10^{th} ed. 2001); and HANDBOOK OF PHARMACEUTICAL EXCIPIENTS (Raymond C. Rowe et al., APhA Publications, 4^{th} edition 2003) which are hereby incorporated by reference in their entirety. These protocols are routine procedures and any modifications are well within the scope of one skilled in the art and from the teaching herein.

It is further envisioned that a pharmaceutical composition disclosed in the present specification can optionally include, without limitation, other pharmaceutically acceptable components, including, without limitation, buffers, preservatives, tonicity adjusters, salts, antioxidants, physiological substances, pharmacological substances, bulking agents, emulsifying agents, wetting agents, sweetening or flavoring agents, and the like. Various buffers and means for adjusting pH can be used to prepare a pharmaceutical composition disclosed in the present specification, provided that the resulting preparation is pharmaceutically acceptable. Such buffers include, without limitation, acetate buffers, citrate buffers, phosphate buffers, neutral buffered saline, phosphate buffered saline and borate buffers. It is understood that acids or bases can be used to adjust the pH of a composition as needed. Pharmaceutically acceptable antioxidants include, without limitation, sodium metabisulfite, sodium thiosulfate, acetylcysteine, butylated hydroxyanisole and butylated hydroxytoluene. Useful preservatives include, without limitation, benzalkonium chloride, chlorobutanol, thimerosal, phenylmercuric acetate, phenylmercuric nitrate and a stabilized oxy chloro composition, for example, PURITE®. Tonicity adjustors useful in a pharmaceutical composition include, without limitation, salts such as, e.g., sodium chloride, potassium chloride, mannitol or glycerin and other pharmaceutically acceptable tonicity adjustor. The pharmaceutical composition may be provided as a salt and can be formed with many acids, including but not limited to, hydrochloric, sulfuric, acetic, lactic, tartaric, malic, succinic, etc. Salts tend to be more soluble in aqueous or other protonic solvents than are the corresponding free base forms. It is understood that these and other substances known in the art of pharmacology can be included in a pharmaceutical composition useful in the invention.

A tolerogizing composition useful in a method of the invention is administered to a human or other mammal in an effective amount. Such an effective amount generally is the minimum dose necessary to achieve the desired therapeutic effect, which can be, for example, that amount roughly necessary to reduce the symptoms associated with a BoNT/A immunoresistant response. For example, the term “effective amount” when used with respect to treating BoNT/A Immunoresistance can be a dose sufficient to the symptoms, for example, by at least 30%, 40%, 50%, 60%, 70%, 80%, 90% or 100%. Such a dose generally is in the range of 0.1-1000 mg/day and can be, for example, in the range of 0.1-500 mg/day, 0.5-500 mg/day, 0.5-100 mg/day, 0.5-50 mg/day, 0.5-20 mg/day, 0.5-10 mg/day or 0.5-5 mg/day, with the actual amount to be administered determined by a physician taking into account the relevant circumstances including the severity of the BoNT/A immunoresistance, the age and weight of the patient, the patient's general physical condition, the cause of the BoNT/A immunoresistance and the route of administration. Where repeated administration is used, the frequency of administration depends, in part, on the half-life of the tolerogizing composition. Suppositories and extended release formulations can be useful in the invention and include, for example, dermal patches, formulations for deposit on or under the skin and formulations for intramuscular injection. It is understood that slow-release formulations also can be useful in the methods of the invention. The subject receiving the tolerogizing composition can be any mammal or other vertebrate capable of experiencing immunoresistance to a BoNT/A treatment, for example, a human, primate, horse, cow, dog, cat or bird.

Various routes of administration can be useful for treating BoNT/A immunoresistance, according to a method of the invention. A pharmaceutical composition useful in the methods of the invention can be administered to a mammal by any of a variety of means depending, for example, on the type and location of BoNT/A immunoresistance to be treated, the BoNT/A tolerogizing composition, or other compound to be included in the composition, and the history, risk factors and symptoms of the subject. Routes of administration suitable for the methods of the invention include both systemic and local administration. As non-limiting examples, a pharmaceutical composition useful for treating BoNT/A immunoresistance can be administered orally or by subcutaneous pump; by dermal patch; by intravenous, subcutaneous or intramuscular injection; by topical drops, creams, gels or ointments; as an implanted or injected extended release formulation; as a bioerodible or non-bioerodible delivery system; by subcutaneous minipump or other implanted device; by intrathecal pump or injection; or by epidural injection. An exemplary list of biodegradable polymers and methods of use are described in, e.g., HANDBOOK OF BIODEGRADABLE POLYMERS (Abraham J. Domb et al., eds., Overseas Publishers Association, 1997); CONTROLLED DRUG DELIVERY: DESIGNING TECHNOLOGIES FOR THE FUTURE (Kinam Park & Randy J. Mrsny eds., American Chemical Association, 2000); Vernon G. Wong, Method for Reducing or Preventing Transplant Rejection in the Eye and Intraocular Implants for Use Therefor, U.S. Pat. No. 6,699,493 (Mar. 2, 2004); Vernon G. Wong & Mae W. L. Hu, Methods for Treating Inflammation-mediated Conditions of the Eye, U.S. Pat. No. 6,726,918 (Apr. 27, 2004); David A. Weber et al., Methods and Apparatus for Delivery of Ocular Implants, U.S. Patent Publication No. US2004/0054374 (Mar. 18, 2004); Thierry Nivaggioli et al., Biodegradable Ocular Implant, U.S. Patent Publication No. US2004/0137059 (Jul.15, 2004), which are hereby incorporated by reference in their entirety. It is understood that the frequency and duration of dosing will be dependent, in part, on the relief desired and the half-life of the tolerogizing composition.

In particular embodiments, a method of the invention is practiced by peripheral administration of a tolerogizing composition. As used herein, the term “peripheral administration” or “administered peripherally” means introducing an agent into a subject outside of the central nervous system. Peripheral administration encompasses any route of administration other than direct administration to the spine or brain. As such, it is clear that intrathecal and epidural administration as well as cranial injection or implantation are not within the scope of the term “peripheral administration” or “administered peripherally.”

Peripheral administration can be local or systemic. Local administration results in significantly more of a pharmaceutical composition being delivered to and about the site of local administration than to regions distal to the site of administration. Systemic administration results in delivery of a pharmaceutical composition to essentially the entire peripheral nervous system of the subject and may also result in delivery to the central nervous system depending on the properties of the composition.

Routes of peripheral administration useful in the methods of the invention encompass, without limitation, oral administration, topical administration, intravenous or other injection, and implanted minipumps or other extended release devices or formulations. A pharmaceutical composition useful in the invention can be peripherally administered, for example, orally in any acceptable form such as in a tablet, liquid, capsule, powder, or the like; by intravenous, intraperitoneal, intramuscular, subcutaneous or parenteral injection; by transdermal diffusion or electrophoresis; topically in any acceptable form such as in drops, creams, gels or ointments; and by minipump or other implanted extended release device or formulation.

VIII. Method for Vaccinating an Individual Against BoNT

A vaccine of the invention can stimulate an immune response against botulinum toxin in a human or other mammal, resulting in the production of antibodies that bind to and neutralize botulinum toxin. Such an immune response increases the ability of a human or other mammal's immune system to destroy botulinum toxin and thereby prevent harmful effects of botulinum toxin exposure. Thus, the present invention provides a method of preventing or reducing botulinum toxicity in a human or other mammal by administering to the human or other mammal a vaccine composition disclosed in the present specification comprising a BoNT/A peptide disclosed in the present specification.

Thus, the present invention provides a method of preventing or reducing BoNT/A toxicity in a human or other mammal by administering to a human or other mammal a vaccine composition disclosed in the present specification. A vaccine composition can be administered to a human or other mammal prior to Botulinum toxin exposure to reduce or prevent BoNT/A toxicity, or after exposure to a Botulinum toxin, for example, when symptoms of toxicity are first apparent. In addition, a vaccine composition can be administered to a human or other mammal who is at increased risk for BoNT/A toxicity. Those skilled in the art will be able to determine an appropriate candidate for receiving a vaccine composition of the invention based on, for example, the particular condition to be treated and the presence or likelihood Botulinum toxin exposure. In one embodiment, a method of the present invention is practiced by administering a vaccine composition to a human or other mammal prior to exposure to a Botulinum toxin. Such a human or other mammal can be, for example, an individual at increased risk for exposure to a Botulinum toxin. In another embodiment, a method of the present invention is practiced by administering a vaccine composition after the human or other mammal has been exposed to a Botulinum toxin. In yet another embodiment, a method of the present invention is practiced by administering a vaccine composition to a human or other mammal who has not been diagnosed with Botulinum toxicity. In yet another embodiment, a method of the present invention is practiced by administering a vaccine composition to a human or other mammal who has been diagnosed with Botulinum toxicity.

In an embodiment of the present invention, a BoNT/A peptide useful in a method disclosed in the present specification for preventing or reducing BoNT toxicity has a length of at most 60 amino acids and consisting of at least 5 contiguous amino acids selected from one of the following BoNT/A amino acid sequences: 449-467 of SEQ ID NO:1(N1), 463-481 of SEQ ID NO:1 (N2), 491-509 of SEQ ID NO:1 (N4), 505-523 of SEQ ID NO:1 (N5), 519-537 of SEQ ID NO:1 (N6), 533-551 of SEQ ID NO:1 (N7), 547-565 of SEQ ID NO:1 (N8), 561-579 of SEQ ID NO:1 (N9), 575-593 of SEQ ID NO:1 (N10), 589-607 of SEQ ID NO:1 (N11), 631-649 of SEQ ID NO:1 (N14), 645-663 of SEQ ID NO:1 (N15), 659-677 of SEQ ID NO:1 (N16), 673-691 of SEQ ID NO:1 (N17), 701-719 of SEQ ID NO:1 (N19), 715-733 of SEQ ID NO:1 (N20), 729-747 of SEQ ID NO:1 (N21), 743-761 of SEQ ID NO:1 (N22), 757-775 of SEQ ID NO:1 (N23), 771-789 of SEQ ID NO:1 (N24), 785-803 of SEQ ID NO:1 (N25), 799-817 of SEQ ID NO:1 (N26), 813-831 of SEQ ID NO:1 (N27), 827-845 of SEQ ID NO:1 (N28), 869-887 of SEQ ID NO:1 (C2), 883-901 of SEQ ID NO:1 (C3), 911-929 of SEQ ID NO:1 (C5), 925-943 of SEQ ID NO:1 (C6), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1065-1083 of SEQ ID NO:1 (C16), 1079-1097 of SEQ ID NO:1 (C17), 1107-1125 of SEQ ID NO:1 (C19), 1121-1139 of SEQ ID NO:1 (C20), 1135-1153 of SEQ ID NO:1 (C21), 1149-1167 of SEQ ID NO:1 (C22), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28), 1247-1265 of SEQ ID NO:1 (C29), 1261-1279 of SEQ ID NO:1 (C30), or 1275-1296 of SEQ ID NO:1 (C31), or a conservative variant or immunoreactive fragment thereof, with the proviso that the BoNT/A peptide is not SEQ ID NO:2.

In another embodiment of the present invention, a BoNT/A peptide useful in a method disclosed in the present specification for preventing or reducing BoNT toxicity has a length of at most 60 amino acids and consisting of at least 5 contiguous amino acids selected from one of the following BoNT/A amino acid sequences: 449-467 of SEQ ID NO:1 (N1), 463-481 of SEQ ID NO:1 (N2), 491-509 of SEQ ID NO:1 (N4), 505-523 of SEQ ID NO:1 (N5), 519-537 of SEQ ID NO:1 (N6), 533-551 of SEQ ID NO:1 (N7), 547-565 of SEQ ID NO:1 (N8), 561-579 of SEQ ID NO:1 (N9), 575-593 of SEQ ID NO:1 (N10), 589-607 of SEQ ID NO:1 (N11), 631-649 of SEQ ID NO:1 (N14), 645-663 of SEQ ID NO:1 (N15), 659-677 of SEQ ID NO:1 (N16), 673-691 of SEQ ID NO:1 (N17), 701-719 of SEQ ID NO:1 (N19), 715-733 of SEQ ID NO:1 (N20), 729-747 of SEQ ID NO:1 (N21), 743-761 of SEQ ID NO:1 (N22), 757-775 of SEQ ID NO:1 (N23), 771-789 of SEQ ID NO:1 (N24), 785-803 of SEQ ID NO:1 (N25), 799-817 of SEQ ID NO:1 (N26), 813-831 of SEQ ID NO:1 (N27), 827-845 of SEQ ID NO:1 (N28), 869-887 of SEQ ID NO:1 (C2), 883-901 of SEQ ID NO:1 (C3), 911-929 of SEQ ID NO:1 (C5), 925-943 of SEQ ID NO:1 (C6), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1065-1083 of SEQ ID NO:1 (C16), 1079-1097 of SEQ ID NO:1 (C17), 1107-1125 of SEQ ID NO:1 (C19), 1121-1139 of SEQ ID NO:1 (C20), 1135-1153 of SEQ ID NO:1 (C21), 1149-1167 of SEQ ID NO:1 (C22), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28), 1247-1265 of SEQ ID NO:1 (C29), 1261-1279 of SEQ ID NO:1 (C30), or 1275-1296 of SEQ ID NO:1 (C31). In an aspect of this embodiment, such a BoNT/A peptide is selected from one of the following amino acid sequences: 463-481 of SEQ ID NO:1 (N2), 519-537 of SEQ ID NO:1 (N6), 533-551 of SEQ ID NO:1 (N7), 547-565 of SEQ ID NO:1 (N8), 561-579 of SEQ ID NO:1 (N9), 589-607 of SEQ ID NO:1 (N11), 659-677 of SEQ ID NO:1 (N16), 743-761 of SEQ ID NO:1 (N22), 785-803 of SEQ ID NO:1 (N25), 813-831 of SEQ ID NO:1 (N27), 827-845 of SEQ ID NO:1 (N28), 869-887 of SEQ ID NO:1 (C2), 883-901 of SEQ ID NO:1(C3), 911-929 of SEQ ID NO:1(C6), 939-957 of SEQ ID NO:1(C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1121-1139 of SEQ ID NO:1 (C20), 1135-1153 of SEQ ID NO:1 (C21), 1177-1195 of SEQ ID NO:1 (C24), 1261-1279 of SEQ ID NO:1 (C30) or 1275-1296 of SEQ ID NO:1 (C31). In another aspect of this embodiment, such a BoNT/A peptide is selected from one of the following amino acid sequences: 463-481 of SEQ ID NO:1 (N2), 533-551 of SEQ ID NO:1 (N7), 547-565 of SEQ ID NO:1 (N8), 561-579 of SEQ ID NO:1 (N9), 659-677 of SEQ ID NO:1 (N16),743-761 of SEQ ID NO:1 (N22), 785-803 of SEQ ID NO:1 (N25), 813-831 of SEQ ID NO:1 (N27), 883-901 of SEQ ID NO:1 (C3), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1121-1139 of SEQ ID NO:1 (C20) or 1177-1195 of SEQ ID NO:1 (C24) or 1275-1296 of SEQ ID NO: 1 (C31). In another aspect of this embodiment, such a BoNT/A peptide is selected from one of the following amino acid sequences: 533-551 of SEQ ID NO: 1 (N7), 547-565 of SEQ ID NO: 1 (N8), 743-761 of SEQ ID NO: 1 (N22), 785-803 of SEQ ID NO: 1 (N25), 813-831 of SEQ ID NO: 1 (N27), 995-1013 of SEQ ID NO: 1 (C11); 1051-1069 of SEQ ID NO: 1 (C15), 1177-1195 of SEQ ID NO: 1 (C24), or 1275-1296 of SEQ ID NO: 1 (C31).

In yet another aspect of this embodiment, a BoNT/A peptide useful in a method disclosed in the present specification for preventing or reducing BoNT toxicity has a length of at most 60 amino acids and consisting of at least 5 contiguous amino acids selected from one of the following BoNT/A amino acid sequences: 463-481 of SEQ ID NO:1 (N2), 505-523 of SEQ ID NO:1 (N5), 519-537 of SEQ ID NO:1 (N6), 533-551 of SEQ ID NO:1 (N7), 603-621 of SEQ ID NO:1 (N12), 645-663 of SEQ ID NO:1 (N15), 659-677 of SEQ ID NO:1 (N16), 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1079-1097 of SEQ ID NO:1 (C17), 1107-1125 of SEQ ID NO:1 (C19), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28) or 1275-1296 of SEQ ID NO:1 (C31). In yet another aspect of this embodiment, such a BoNT/A peptide is selected from one of the following amino acid sequences: 533-551 of SEQ ID NO:1 (N7), 659-677 of SEQ ID NO:1 (N16), 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1107-1125 of SEQ ID NO:1 (C19), 1163-1181 of SEQ ID NO:1 (C23), 1191-1209 of SEQ ID NO:1 (C25), 1233-1251 of SEQ ID NO:1 (C28) or 1275-1296 of SEQ ID NO:1 (C31). In yet another aspect of this embodiment, such a BoNT/A peptide is selected from one of the following amino acid sequences: 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1079-1097 of SEQ ID NO:1 (C17), 1107-1125 of SEQ ID NO:1 (C19), 1163-1181 of SEQ NO:1 (C23), 1177-1195 of SEQ ID NO:1 (C24), 1191-1209 of SEQ ID NO:1 (C25), 1233-1251 of SEQ ID NO:1 (C28) or 1275-1296 of SEQ ID NO:1 (C31). In yet another aspect of this embodiment, such a BoNT/A peptide is selected from one of the following amino acid sequences: 729-747 of SEQ ID NO:1 (N21) 799-817 of SEQ ID NO:1 (N26), 1065-1083 of SEQ ID NO:1 (C16), 1163-1181 of SEQ ID NO:1 (C23) or 1275-1296 of SEQ ID NO:1 (C31).

In yet another embodiment of the present invention, a BoNT/A peptide useful in a method disclosed in the present specification for preventing or reducing BoNT/A toxicity is selected from one of the following BoNT/A amino acid sequences: 449-467 of SEQ ID NO:1(N1), 463-481 of SEQ ID NO:1 (N2), 491-509 of SEQ ID NO:1 (N4), 505-523 of SEQ ID NO:1 (N5), 519-537 of SEQ ID NO:1 (N6), 533-551 of SEQ ID NO:1 (N7), 547-565 of SEQ ID NO:1 (N8), 561-579 of SEQ ID NO:1 (N9), 575-593 of SEQ ID NO:1 (N10), 589-607 of SEQ ID NO:1 (N11), 631-649 of SEQ ID NO:1 (N14), 645-663 of SEQ ID NO:1 (N15), 659-677 of SEQ ID NO:1 (N16), 673-691 of SEQ ID NO:1 (N17), 701-719 of SEQ ID NO:1 (N19), 715-733 of SEQ ID NO:1 (N20), 729-747 of SEQ ID NO:1 (N21), 743-761 of SEQ ID NO:1 (N22), 757-775 of SEQ ID NO:1 (N23), 771-789 of SEQ ID NO:1 (N24), 785-803 of SEQ ID NO:1 (N25), 799-817 of SEQ ID NO:1 (N26), 813-831 of SEQ ID NO:1 (N27), 827-845 of SEQ ID NO:1 (N28), 869-887 of SEQ ID NO:1 (C2), 883-901 of SEQ ID NO:l (C3), 911-929 of SEQ ID NO:1 (C5), 925-943 of SEQ ID NO:1 (C6), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1065-1083 of SEQ ID NO:1 (C16), 1079-1097 of SEQ ID NO:1 (C17), 1107-1125 of SEQ ID NO:1 (C19), 1121-1139 of SEQ ID NO:1 (C20), 1135-1153 of SEQ ID NO:1 (C21), 1149-1167 of SEQ ID NO:1 (C22), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28), 1247-1265 of SEQ ID NO:1 (C29), 1261-1279 of SEQ ID NO:1 (C30), or 1275-1296 of SEQ ID NO:1 (C31). In an aspect of this embodiment, such a BoNT/A peptide is selected from one of the following amino acid sequences: 463-481 of SEQ ID NO:1 (N2), 519-537 of SEQ ID NO:1 (N6), 533-551 of SEQ ID NO:1 (N7), 547-565 of SEQ ID NO:1 (N8), 561-579 of SEQ ID NO:1 (N9), 589-607 of SEQ ID NO:1 (N11), 659-677 of SEQ ID NO:1 (N16), 743-761 of SEQ ID NO:l (N22), 785-803 of SEQ ID NO:1 (N25), 813-831 of SEQ ID NO:1 (N27), 827-845 of SEQ ID NO:1 (N28), 869-887 of SEQ ID NO:1 (C2), 883-901 of SEQ ID NO:1 (C3), 911-929 of SEQ ID NO:1 (C6), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1121-1139 of SEQ ID NO:1 (C20), 1135-1153 of SEQ ID NO:1 (C21), 1177-1195 of SEQ ID NO:1 (C24), 1261-1279 of SEQ ID NO:1 (C30) or 1275-1296 of SEQ ID NO:1 (C31). In another aspect of this embodiment, such a BoNT/A peptide is selected from one of the following amino acid sequences: 463-481 of SEQ ID NO:1 (N2), 533-551 of SEQ ID NO:1 (N7), 547-565 of SEQ ID NO:1 (N8), 561-579 of SEQ ID NO:1 (N9), 659-677 of SEQ ID NO:1 (N16), 743-761 of SEQ ID NO:1 (N22), 785-803 of SEQ ID NO:1 (N25), 813-831 of SEQ ID NO:1 (N27), 883-901 of SEQ ID NO:1 (C3), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1121-1139 of SEQ ID NO:1 (C20) or 1177-1195 of SEQ ID NO:1 (C24) or 1275-1296 of SEQ ID NO: 1 (C31). In another aspect of this embodiment, such a BoNT/A peptide is selected from one of the following amino acid sequences: 533-551 of SEQ ID NO: 1 (N7), 547-565 of SEQ ID NO: 1 (N8), 743-761 of SEQ ID NO: 1 (N22), 785-803 of SEQ ID NO: 1 (N25), 813-831 of SEQ ID NO: 1 (N27), 995-1013 of SEQ ID NO: 1 (C11); 1051-1069 of SEQ ID NO: 1 (Cl 5),1177-1195 of SEQ ID NO: 1 (C24), or 1275-1296 of SEQ ID NO: 1 (C31).

In yet another aspect of this embodiment, a BoNT/A peptide useful in a method disclosed in the present specification for preventing or reducing BoNT toxicity is selected from one of the following BoNT/A amino acid sequences: 463-481 of SEQ ID NO:1 (N2), 505-523 of SEQ ID NO:1 (N5), 519-537 of SEQ ID NO:1 (N6), 533-551 of SEQ ID NO:1 (N7), 603-621 of SEQ ID NO:1 (N12), 645-663 of SEQ ID NO:1 (N15), 659-677 of SEQ ID NO:1 (N16), 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1079-1097 of SEQ ID NO:1 (C17), 1107-1125 of SEQ ID NO:1 (C19), 1163-1181 of SEQ ID NO:1 (C23), 1177-1195 of SEQ ID NO:1 (C24), 1191-1209 of SEQ ID NO:1 (C25), 1233-1251 of SEQ ID NO:l (C28) or 1275-1296 of SEQ ID NO:1 (C31). In yet another aspect of this embodiment, such a BoNT/A peptide is selected from one of the following amino acid sequences: 533-551 of SEQ ID NO:1 (N7), 659-677 of SEQ ID NO:1 (N16), 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16),1107-1125 of SEQ ID NO:1 (C19),1163-1181 of SEQ ID NO:1 (C23),1191-1209 of SEQ ID NO:1 (C25), 1233-1251 of SEQ ID NO:1 (C28) or 1275-1296 of SEQ ID NO:1 (C31). In yet another aspect of this embodiment, such a BoNT/A peptide is selected from one of the following amino acid sequences: 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1079-1097 of SEQ ID NO:1 (C17), 1107-1125 of SEQ ID NO:1 (C19), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28) or 1275-1296 of SEQ ID NO:1 (C31). In yet another aspect of this embodiment, such a BoNT/A peptide is selected from one of the following amino acid sequences: 729-747 of SEQ ID NO:1 (N21) 799-817 of SEQ ID NO:1 (N26), 1065-1083 of SEQ ID NO:1 (C16),1163-1181 of SEQ ID NO:1 (C23) or 1275-1296 of SEQ ID NO:1 (C31).

In yet another embodiment of the present invention, a BoNT/A peptide useful in a method disclosed in the present specification for preventing or reducing BoNT toxicity has a length of at most 60 amino acids and consisting of at least 5 contiguous amino acids selected from one of the following BoNT/A amino acid sequences: 449-467 of SEQ ID NO:1(N1), 463-481 of SEQ ID NO:1 (N2), 491-509 of SEQ ID NO:1 (N4), 505-523 of SEQ ID NO:1 (N5), 519-537 of SEQ ID NO:1 (N6), 533-551 of SEQ ID NO:1 (N7), 547-565 of SEQ ID NO:1 (N8), 561-579 of SEQ ID NO:1 (N9), 575-593 of SEQ ID NO:1 (N10), 589-607 of SEQ ID NO:1 (N11), 631-649 of SEQ ID NO:1 (N14), 645-663 of SEQ ID NO:1 (N15), 659-677 of SEQ ID NO:1 (N16), 673-691 of SEQ ID NO:1 (N17), 701-719 of SEQ ID NO:1 (N19), 715-733 of SEQ ID NO:1 (N20), 729-747 of SEQ ID NO:1 (N21), 743-761 of SEQ ID NO:1 (N22), 757-775 of SEQ ID NO:1 (N23), 771-789 of SEQ ID NO:1 (N24), 785-803 of SEQ ID NO:1 (N25), 799-817 of SEQ ID NO:1 (N26), 813-831 of SEQ ID NO:1 (N27), 827-845 of SEQ ID NO:1 (N28), 869-887 of SEQ ID NO:1 (C2), 883-901 of SEQ ID NO:1 (C3), 911-929 of SEQ ID NO:1 (C5), 925-943 of SEQ ID NO:1 (C6), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1065-1083 of SEQ ID NO:1 (C16), 1079-1097 of SEQ ID NO:1 (C17), 1107-1125 of SEQ ID NO:1 (C19), 1121-1139of SEQ ID NO:1 (C20), 1135-1153 of SEQ ID NO:1 (C21), 1149-1167of SEQ ID NO:1 (C22), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28), 1247-1265 of SEQ ID NO:1 (C29), 1261-1279 of SEQ ID NO:1 (C30), or 1275-1296 of SEQ ID NO:1 (C31), or a conservative variant thereof. In an aspect of this embodiment, such a BoNT/A peptide is selected from one of the following amino acid sequences: 463-481 of SEQ ID NO:1 (N2), 519-537 of SEQ ID NO:1 (N6), 533-551 of SEQ ID NO:1 (N7), 547-565 of SEQ ID NO:1 (N8), 561-579 of SEQ ID NO:1 (N9), 589-607 of SEQ ID NO:1 (N11), 659-677 of SEQ ID NO:1 (N16), 743-761 of SEQ ID NO:1 (N22), 785-803 of SEQ ID NO:1 (N25), 813-831 of SEQ ID NO:1 (N27), 827-845 of SEQ ID NO:1 (N28), 869-887 of SEQ ID NO:1 (C2), 883-901 of SEQ ID NO:1 (C3), 911-929 of SEQ ID NO:1 (C6), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1121-1139 of SEQ ID NO:1 (C20), 1135-1153 of SEQ ID NO:1 (C21), 1177-1195 of SEQ ID NO:1 (C24), 1261-1279 of SEQ ID NO:1 (C30) or 1275-1296 of SEQ ID NO:1 (C31), or a conservative variant thereof. In another aspect of this embodiment, such a BoNT/A peptide is selected from one of the following amino acid sequences: 463-481 of SEQ ID NO:1 (N2), 533-551 of SEQ ID NO:1 (N7), 547-565 of SEQ ID NO:1 (N8), 561-579 of SEQ ID NO:1 (N9), 659-677 of SEQ ID NO:1 (N16), 743-761 of SEQ ID NO:1 (N22), 785-803 of SEQ ID NO:1 (N25), 813-831 of SEQ ID NO:1 (N27), 883-901 of SEQ ID NO:1 (C3), 939-957 of SEQ ID NO:1 (C7), 967-985 of SEQ ID NO:1 (C9), 981-999 of SEQ ID NO:1 (C1o), 995-1013 of SEQ ID NO:1 (C11), 1051-1069 of SEQ ID NO:1 (C15), 1121-1139 of SEQ ID NO:1 (C20) or 1177-1195 of SEQ ID NO:1 (C24) or 1275-1296 of SEQ ID NO: 1 (C31), or a conservative variant thereof. In another aspect of this embodiment, such a BoNT/A peptide is selected from one of the following amino acid sequences: 533-551 of SEQ ID NO: 1 (N7), 547-565 of SEQ ID NO: 1 (N8), 743-761 of SEQ ID NO: 1 (N22), 785-803 of SEQ ID NO: 1 (N25), 813-831 of SEQ ID NO: 1 (N27), 995-1013 of SEQ ID NO: 1 (C11); 1051-1069 of SEQ ID NO: 1 (C15), 1177-1195 of SEQ ID NO: 1 (C24), or 1275-1296 of SEQ ID NO: 1 (C31), or a conservative variant thereof.

In yet another aspect of this embodiment, a BoNT/A peptide useful in a method disclosed in the present specification for preventing or reducing BoNT toxicity has a length of at most 60 amino acids and consisting of at least 5 contiguous amino acids selected from one of the following BoNT/A amino acid sequences: 463-481 of SEQ ID NO:1 (N2), 505-523 of SEQ ID NO:1 (N5), 519-537 of SEQ ID NO:1 (N6), 533-551 of SEQ ID NO:1 (N7), 603-621 of SEQ ID NO:1 (N12), 645-663 of SEQ ID NO:1 (N15), 659-677 of SEQ ID NO:1 (N16), 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1079-1097 of SEQ ID NO:1 (C17), 1107-1125 of SEQ ID NO:1 (C19), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28) or 1275-1296 of SEQ ID NO:1 (C31), or a conservative variant thereof. In yet another aspect of this embodiment, such a BoNT/A peptide is selected from one of the following amino acid sequences: 533-551 of SEQ ID NO:1 (N7), 659-677 of SEQ ID NO:1 (N16), 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1107-1125 of SEQ ID NO:1 (C19), 1163-1181 of SEQ ID NO:1 (C23), 1191-1209 of SEQ ID NO:1 (C25), 1233-1251 of SEQ ID NO:1 (C28) or 1275-1296 of SEQ ID NO:1 (C31), or a conservative variant thereof. In yet another aspect of this embodiment, such a BoNT/A peptide is selected from one of the following amino acid sequences: 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1079-1097 of SEQ ID NO:1 (C17), 1107-1125 of SEQ ID NO:1 (C19), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28) or 1275-1296 of SEQ ID NO:1 (C31), or a conservative variant thereof. In yet another aspect of this embodiment, such a BoNT/A peptide is selected from one of the following amino acid sequences: 729-747 of SEQ ID NO:1 (N21) 799-817 of SEQ ID NO:1 (N26), 1065-1083 of SEQ ID NO:1 (C16), 1163-1181 of SEQ ID NO:1 (C23) or 1275-1296 of SEQ ID NO:1 (C31), or a conservative variant thereof.

In yet another embodiment of the present invention, a BoNT/A peptide useful in a method disclosed in the present specification for preventing or reducing BoNT toxicity has a length of at most 60 amino acids and consisting of at least 5 contiguous amino acids selected from one of the following BoNT/A amino acid sequences: 449-467 of SEQ ID NO:1 (N1), 463-481 of SEQ ID NO:1 (N2), 491-509 of SEQ ID NO:1 (N4), 505-523 of SEQ ID NO:1 (N5), 519-537 of SEQ ID NO:1 (N6), 533-551 of SEQ ID NO:1 (N7), 547-565 of SEQ ID NO:1 (N8), 561-579 of SEQ ID NO:1 (N9), 575-593 of SEQ ID NO:1 (N10), 589-607 of SEQ ID NO:1 (N11), 631-649 of SEQ ID NO:1 (N14), 645-663 of SEQ ID NO:1 (N15), 659-677 of SEQ ID NO:1 (N16), 673-691 of SEQ ID NO:1 (N17), 701-719 of SEQ ID NO:1 (N19), 715-733 of SEQ ID NO:1 (N20), 729-747 of SEQ ID NO:1 (N21), 743-761 of SEQ ID NO:1 (N22), 757-775 of SEQ ID NO:1 (N23), 771-789 of SEQ ID NO:1 (N24), 785-803 of SEQ ID NO:1 (N25), 799-817 of SEQ ID NO:1 (N26), 813-831 of SEQ ID NO:1 (N27), 827-845 of SEQ ID NO:1 (N28), 869-887 of SEQ ID NO:1 (C2), 883-901 of SEQ ID NO:1 (C3), 911-929 of SEQ ID NO:l (C5), 925-943 of SEQ ID NO:1 (C6), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1065-1083 of SEQ ID NO:1 (C16), 1079-1097 of SEQ ID NO:1 (C17), 1107-1125 of SEQ ID NO:1 (C19), 1121-1139 of SEQ ID NO:1 (C20), 1135-1153 of SEQ ID NO:1 (C21), 1149-1167 of SEQ ID NO:l (C22), 1163-1181 of SEQ ID NO:l (C23), 1177-1195 of SEQ ID NO:l (C24), 1191-1209 of SEQ ID NO:1 (C25), 1233-1251 of SEQ ID NO:1 (C28), 1247-1265 of SEQ ID NO:1 (C29), 1261-1279 of SEQ ID NO:1 (C30), or 1275-1296 of SEQ ID NO:1 (C31), or a non-conservative variant thereof. In an aspect of this embodiment, such a BoNT/A peptide is selected from one of the following amino acid sequences: 463-481 of SEQ ID NO:1 (N2), 519-537 of SEQ ID NO:1 (N6), 533-551 of SEQ ID NO:1 (N7), 547-565 of SEQ ID NO:1 (N8), 561-579 of SEQ ID NO:1 (N9), 589-607 of SEQ ID NO:1 (N11), 659-677 of SEQ ID NO:1 (N16), 743-761 of SEQ ID NO:1 (N22), 785-803 of SEQ ID NO:1 (N25), 813-831 of SEQ ID NO:1 (N27), 827-845 of SEQ ID NO:1 (N28), 869-887 of SEQ ID NO:1 (C2), 883-901 of SEQ ID NO:1 (C3), 911-929 of SEQ ID NO:1 (C6), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1121-1139 of SEQ ID NO:1 (C20), 1135-1153 of SEQ ID NO:1 (C21), 1177-1195 of SEQ ID NO:1 (C24), 1261-1279 of SEQ ID NO:1 (C30) or 1275-1296 of SEQ ID NO:1 (C31), or a non-conservative variant thereof. In another aspect of this embodiment, such a BoNT/A peptide is selected from one of the following amino acid sequences: 463-481 of SEQ ID NO:1 (N2), 533-551 of SEQ ID NO:1 (N7), 547-565 of SEQ ID NO:l (N8), 561-579 of SEQ ID NO:1 (N9), 659-677 of SEQ ID NO:1 (N16), 743-761 of SEQ ID NO:1 (N22), 785-803 of SEQ ID NO:1 (N25), 813-831 of SEQ ID NO:1 (N27), 883-901 of SEQ ID NO:1 (C3), 939-957 of SEQ ID NO:1 (C7), 967-985 of SEQ ID NO:1 (C9), 981-999 of SEQ ID NO:1 (Cl 0), 995-1013 of SEQ ID NO:1 (C11), 1051-1069 of SEQ ID NO:1 (C15), 1121-1139 of SEQ ID NO:1 (C20) or 1177-1195 of SEQ ID NO:1 (C24) or 1275-1296 of SEQ ID NO: 1 (C31), or a non-conservative variant thereof. In another aspect of this embodiment, such a BoNT/A peptide is selected from one of the following amino acid sequences: 533-551 of SEQ ID NO: 1 (N7), 547-565 of SEQ ID NO: 1 (N8), 743-761 of SEQ ID NO: 1 (N22), 785-803 of SEQ ID NO: 1 (N25), 813-831 of SEQ ID NO: 1 (N27), 995-1013 of SEQ ID NO: 1 (C11); 1051-1069 of SEQ ID NO: 1 (C15), 1177-1195 of SEQ ID NO: 1 (C24), or 1275-1296 of SEQ ID NO: 1 (C31), or a non-conservative variant thereof.

In yet another aspect of this embodiment, a BoNT/A peptide useful in a method disclosed in the present specification for preventing or reducing BoNT toxicity has a length of at most 60 amino acids and consisting of at least 5 contiguous amino acids selected from one of the following BoNT/A amino acid sequences: 463-481 of SEQ ID NO:1 (N2), 505-523 of SEQ ID NO:1 (N5), 519-537 of SEQ ID NO:1 (N6), 533-551 of SEQ ID NO:1 (N7), 603-621 of SEQ ID NO:1 (N12), 645-663 of SEQ ID NO:1 (N15), 659-677 of SEQ ID NO:1 (N16), 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1079-1097 of SEQ ID NO:1 (C17), 1107-1125 of SEQ ID NO:1 (C19), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28) or 1275-1296 of SEQ ID NO:1 (C31), or a non-conservative variant thereof. In yet another aspect of this embodiment, such a BoNT/A peptide is selected from one of the following amino acid sequences: 533-551 of SEQ ID NO:1 (N7), 659-677 of SEQ ID NO:1 (N16), 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1107-1125 of SEQ ID NO:1 (C19), 1163-1181 of SEQ ID NO:1 (C23), 1191-1209 of SEQ ID NO:1 (C25), 1233-1251 of SEQ ID NO:1 (C28) or 1275-1296 of SEQ ID NO:1 (C31), or a non-conservative variant thereof. In yet another aspect of this embodiment, such a BoNT/A peptide is selected from one of the following amino acid sequences: 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1079-1097 of SEQ ID NO:1 (C17), 1107-1125 of SEQ ID NO:1 (C19), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28) or 1275-1296 of SEQ ID NO:1 (C31), or a non-conservative variant thereof. In yet another aspect of this embodiment, such a BoNT/A peptide is selected from one of the following amino acid sequences: 729-747 of SEQ ID NO:1 (N21) 799-817 of SEQ ID NO:1 (N26), 1065-1083 of SEQ ID NO:1 (C16), 1163-1181 of SEQ ID NO:1 (C23) or 1275-1296 of SEQ ID NO:1 (C31), or a non-conservative variant thereof.

In yet another embodiment of the present invention, a BoNT/A peptide useful in a method disclosed in the present specification for preventing or reducing BoNT toxicity has a length of at most 60 amino acids and consisting of at least 5 contiguous amino acids selected from one of the following BoNT/A amino acid sequences: 449-467 of SEQ ID NO:1(N1), 463-481 of SEQ ID NO:1 (N2), 491-509 of SEQ ID NO:1 (N4), 505-523 of SEQ ID NO:1 (N5), 519-537 of SEQ ID NO:1 (N6), 533-551 of SEQ ID NO:1 (N7), 547-565 of SEQ ID NO:1 (N8), 561-579 of SEQ ID NO:1 (N9), 575-593 of SEQ ID NO:1 (N10), 589-607 of SEQ ID NO:1 (N11), 631-649 of SEQ ID NO:1 (N14), 645-663 of SEQ ID NO:1 (N15), 659-677 of SEQ ID NO:1 (N16), 673-691 of SEQ ID NO:1 (N17), 701-719 of SEQ ID NO:1 (N19), 715-733 of SEQ ID NO:1 (N20), 729-747 of SEQ ID NO:1 (N21), 743-761 of SEQ ID NO:1 (N22), 757-775 of SEQ ID NO:1 (N23), 771-789 of SEQ ID NO:1 (N24), 785-803 of SEQ ID NO:1 (N25), 799-817 of SEQ ID NO:1 (N26), 813-831 of SEQ ID NO:1 (N27), 827-845 of SEQ ID NO:1 (N28), 869-887 of SEQ ID NO:1 (C2), 883-901 of SEQ ID NO:1 (C3), 911-929 of SEQ ID NO:1 (C5), 925-943 of SEQ ID NO:1 (C6), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1065-1083 of SEQ ID NO:1 (C16), 1079-1097 of SEQ ID NO:1 (C17), 1107-1125 of SEQ ID NO:1 (C19), 1121-1139 of SEQ ID NO:1 (C20), 1135-1153 of SEQ ID NO:l (C21), 1149-1167 of SEQ ID NO:1 (C22), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28), 1247-1265 of SEQ ID NO:1 (C29), 1261-1279 of SEQ ID NO:1 (C30), or 1275-1296 of SEQ ID NO:1 (C31), or an immunoreactive fragment thereof, with the proviso that the BoNT/A peptide is not SEQ ID NO:2. In an aspect of this embodiment, such a BoNT/A peptide is selected from one of the following amino acid sequences: 463-481 of SEQ ID NO:1 (N2), 519-537 of SEQ ID NO:1 (N6), 533-551 of SEQ ID NO:1 (N7), 547-565 of SEQ ID NO:1 (N8), 561-579 of SEQ ID NO:1 (N9), 589-607 of SEQ ID NO:1 (N11), 659-677 of SEQ ID NO:1 (N16), 743-761 of SEQ ID NO:1 (N22), 785-803 of SEQ ID NO:1 (N25), 813-831 of SEQ ID NO:1 (N27), 827-845 of SEQ ID NO:1 (N28), 869-887 of SEQ ID NO:1 (C2), 883-901 of SEQ ID NO:1 (C3), 911-929 of SEQ ID NO:1 (C6), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1121-1139 of SEQ ID NO:1 (C20), 1135-1153 of SEQ ID NO:1 (C21), 1177-1195 of SEQ ID NO:1 (C24), 1261-1279 of SEQ ID NO:1 (C30) or 1275-1296 of SEQ ID NO:1 (C31), or an immunoreactive fragment thereof, with the proviso that the BoNT/A peptide is not SEQ ID NO:2. In another aspect of this embodiment, such a BoNT/A peptide is selected from one of the following amino acid sequences: 463-481 of SEQ ID NO:1 (N2), 533-551 of SEQ ID NO:1 (N7), 547-565 of SEQ ID NO:1 (N8), 561-579 of SEQ ID NO:1 (N9), 659-677 of SEQ ID NO:1 (N16), 743-761 of SEQ ID NO:1 (N22), 785-803 of SEQ ID NO:1 (N25), 813-831 of SEQ ID NO:1 (N27), 883-901 of SEQ ID NO:1 (C3), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1121-1139 of SEQ ID NO:1 (C20) or 1177-1195 of SEQ ID NO:1 (C24) or 1275-1296 of SEQ ID NO: 1 (C31), or an immunoreactive fragment thereof, with the proviso that the BoNT/A peptide is not SEQ ID NO:2. In another aspect of this embodiment, such a BoNT/A peptide is selected from one of the following amino acid sequences: 533-551 of SEQ ID NO: 1 (N7), 547-565 of SEQ ID NO: 1 (N8), 743-761 of SEQ ID NO: 1 (N22), 785-803 of SEQ ID NO: 1 (N25), 813-831 of SEQ ID NO: 1 (N27), 995-1013 of SEQ ID NO: 1 (C11); 1051-1069 of SEQ ID NO: 1 (C15), 1177-1195 of SEQ ID NO: 1 (C24), or 1275-1296 of SEQ ID NO: 1 (C31), or an immunoreactive fragment thereof, with the proviso that the BoNT/A peptide is not SEQ ID NO:2.

In yet another aspect of this embodiment, a BoNT/A peptide useful in a method disclosed in the present specification for preventing or reducing BoNT toxicity has a length of at most 60 amino acids and consisting of at least 5 contiguous amino acids selected from one of the following BoNT/A amino acid sequences: 463-481 of SEQ ID NO:1 (N2), 505-523 of SEQ ID NO:1 (N5), 519-537 of SEQ ID NO:1 (N6), 533-551 of SEQ ID NO:1 (N7), 603-621 of SEQ ID NO:1 (N12), 645-663 of SEQ ID NO:1 (N15), 659-677 of SEQ ID NO:1 (N16), 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1079-1097 of SEQ ID NO:1 (C17), 1107-1125 of SEQ ID. NO:1 (C19), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28) or 1275-1296 of SEQ ID NO:1 (C31), or an immunoreactive fragment thereof, with the proviso that the BoNT/A peptide is not SEQ ID NO:2. In yet another aspect of this embodiment, such a BoNT/A peptide is selected from one of the following amino acid sequences: 533-551 of SEQ ID NO:1 (N7), 659-677 of SEQ ID NO:1 (N16), 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1107-1125 of SEQ ID NO:1 (C19), 1163-1181 of SEQ ID NO:1 (C23), 1191-1209 of SEQ ID NO:1 (C25), 1233-1251 of SEQ ID NO:1 (C28) or 1275-1296 of SEQ ID NO:1 (C31), or an immunoreactive fragment thereof, with the proviso that the BoNT/A peptide is not SEQ ID NO:2. In yet another aspect of this embodiment, such a BoNT/A peptide is selected from one of the following amino acid sequences: 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1079-1097 of SEQ ID NO:1 (C17), 1107-1125 of SEQ ID NO:1 (C19), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28) or 1275-1296 of SEQ ID NO:1 (C31), or an immunoreactive fragment thereof, with the proviso that the BoNT/A peptide is not SEQ ID NO:2. In yet another aspect of this embodiment, such a BoNT/A peptide is selected from one of the following amino acid sequences: 729-747 of SEQ ID NO:1 (N21) 799-817 of SEQ ID NO:1 (N26), 1065-1083 of SEQ ID NO:1 (C16), 1163-1181 of SEQ ID NO:1 (C23) or 1275-1296 of SEQ ID NO:1 (C31), or an immunoreactive fragment thereof, with the proviso that the BoNT/A peptide is not SEQ ID NO:2.

In is envisioned that a BoNT/A peptide useful in a method disclosed in the present specification for preventing or reducing BONT toxicity can have any of a variety of lengths from at least 5 amino acids to at most 60 amino acids. Therefore, aspects of this embodiment may include a BoNT/A peptide with at least, e.g., five amino acids, six amino acids, seven amino acids, eight amino acids, nine amino acids, ten amino acids, 11 amino acids, 12 amino acids, 13 amino acids, 14 amino acids, 15 amino acids, 16 amino acids, 17 amino acids, 18 amino acids, 19 amino acids, 20 amino acids, 25 amino acids, 30 amino acids, 35 amino acids, 40 amino acids, 45 amino acids, 50 amino acids, 55 amino acids or 60 amino acids. Other aspects of this embodiment may include a BoNT/A peptide with at least, e.g., five amino acids of SEQ ID NO:1, six amino acids of SEQ ID NO:1, seven amino acids of SEQ ID NO:1, eight amino acids of SEQ ID NO:1, nine amino acids of SEQ ID NO:1, ten amino acids of SEQ ID NO:1, 11 amino acids of SEQ ID NO:1, 12 amino acids of SEQ ID NO:1, 13 amino acids of SEQ ID NO:1, 14 amino acids of SEQ ID NO:1, 15 amino acids of SEQ ID NO:1, 16 amino acids of SEQ ID NO:1, 17 amino acids of SEQ ID NO:1, 18 amino acids of SEQ ID NO:1, 19 amino acids of SEQ ID NO:1, 20 amino acids of SEQ ID NO:1, 25 amino acids of SEQ ID NO:1, 30 amino acids of SEQ ID NO:1, 35 amino acids of SEQ ID NO:1, 40 amino acids of SEQ ID NO:1, 45 amino acids of SEQ ID NO:1, 50 amino acids of SEQ ID NO:1, 55 amino acids or 60 amino acids of SEQ ID NO:1. In further embodiments, such a BoNT/A peptide of the invention may include a BoNT/A peptide with at least, e.g., five amino acids, six amino acids, seven amino acids, eight amino acids, nine amino acids, ten amino acids, 11 amino acids, 12 amino acids, 13 amino acids, 14 amino acids, 15 amino acids, 16 amino acids, 17 amino acids, 18 amino acids, 19 amino acids, 20 amino acids, 25 amino acids, 30 amino acids, 35 amino acids, 40 amino acids, 45 amino acids, 50 amino acids, 55 amino acids or 60 amino acids and consist of at least 5 contiguous amino acids selected from one of the following BoNT/A amino acid sequences: 449-467 of SEQ ID NO:1(N1), 463-481 of SEQ ID NO:1 (N2), 491-509 of SEQ ID NO:1 (N4), 505-523 of SEQ ID NO:1 (N5), 519-537 of SEQ ID NO:1 (N6), 533-551 of SEQ ID NO:1 (N7), 547-565 of SEQ ID NO:1 (N8), 561-579 of SEQ ID NO:1 (N9), 575-593 of SEQ ID NO:1 (N10), 589-607 of SEQ ID NO:1 (N11), 631-649 of SEQ ID NO:1 (N14), 645-663 of SEQ ID NO:1 (N15), 659-677 of SEQ ID NO:1 (N16), 673-691 of SEQ ID NO:1 (N17), 701-719 of SEQ ID NO:1 (N19), 715-733 of SEQ ID NO:1 (N20), 729-747 of SEQ ID NO:1 (N21), 743-761 of SEQ ID NO:1 (N22), 757-775 of SEQ ID NO:1 (N23), 771-789 of SEQ ID NO:1 (N24), 785-803 of SEQ ID NO:1 (N25), 799-817 of SEQ ID NO:1 (N26), 813-831 of SEQ ID NO:1 (N27), 827-845 of SEQ ID NO:1 (N28), 869-887 of SEQ ID NO:1 (C2), 883-901 of SEQ ID NO:1 (C3), 911-929 of SEQ ID NO:1 (C5), 925-943 of SEQ ID NO:1 (C6), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1065-1083 of SEQ ID NO:1 (C16), 1079-1097 of SEQ ID NO:1 (C17), 1107-1125 of SEQ ID NO:1 (C19), 1121-1139 of SEQ ID NO:1 (C20), 1135-1153 of SEQ ID NO:1 (C21), 1149-1167 of SEQ ID NO:1 (C22), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28), 1247-1265 of SEQ ID NO:1 (C29), 1261-1279 of SEQ ID NO:1 (C30) or 1275-1296 of SEQ ID NO:1 (C31), or a conservative variant, a non-conservative variant, or immunoreactive fragment thereof, with the proviso that the BoNT/A peptide is not SEQ ID NO:2.

In another embodiment of the present invention, a BoNT/A peptide useful in a method disclosed in the present specification for preventing or reducing BoNT toxicity can comprise one BoNT/A peptide disclosed in the present specification. In another embodiment of the present invention, a BoNT/A peptide useful in a method disclosed in the present specification for preventing or reducing BoNT toxicity can comprise a plurality of BoNT/A peptides disclosed in the present specification. Thus, aspects of this embodiment can include one or more BoNT/A peptides, two or more BoNT/A peptides, three or more BoNT/A peptides, four or more BoNT/A peptides, five or more BoNT/A peptides, six or more BoNT/A peptides, seven or more BoNT/A peptides, eight or more BoNT/A peptides, nine or more BoNT/A peptides, ten or more BoNT/A peptides, 15 or more BoNT/A peptides, 20 or more BoNT/A peptides, 25 or more BoNT/A peptides or 30 or more BoNT/A peptides. In other aspects of this embodiment can include one or more BoNT/A conservative variants, two or more BoNT/A conservative variants, three or more BoNT/A conservative variants, four or more BoNT/A conservative variants, five or more BoNT/A conservative variants, six or more BoNT/A conservative variants, seven or more BoNT/A conservative variants, eight or more BoNT/A conservative variants, nine or more BoNT/A conservative variants, ten or more BoNT/A conservative variants, 15 or more BoNT/A conservative variants, 20 or more BoNT/A conservative variants, 25 or more BoNT/A conservative variants or 30 or more BoNT/A conservative variants. In further aspects of this embodiment can include one or more BoNT/A non-conservative variants, two or more BoNT/A non-conservative variants, three or more BoNT/A non-conservative variants, four or more BoNT/A non-conservative variants, five or more BoNT/A non-conservative variants, six or more BoNT/A non-conservative variants, seven or more BoNT/A non-conservative variants, eight or more BoNT/A non-conservative variants, nine or more BoNT/A non-conservative variants, ten or more BoNT/A non-conservative variants, 15 or more BoNT/A non-conservative variants, 20 or more BoNT/A non-conservative variants, 25 or more BoNT/A non-conservative variants or 30 or more BoNT/A non-conservative variants. In still other aspects of this embodiment can include one or more BoNT/A immunoreactive fragments, two or more BoNT/A immunoreactive fragments, three or more BoNT/A immunoreactive fragments, four or more BoNT/A immunoreactive fragments, five or more BoNT/A immunoreactive fragments, six or more BoNT/A immunoreactive fragments, seven or more BoNT/A immunoreactive fragments, eight or more BoNT/A immunoreactive fragments, nine or more BoNT/A peptides, ten or more BoNT/A immunoreactive fragments, 15 or more BoNT/A immunoreactive fragments, 20 or more BoNT/A immunoreactive fragments, 25 or more BoNT/A immunoreactive fragments or 30 or more BoNT/A immunoreactive fragments. BoNT/A peptides disclosed in the present specification useful for preventing or reducing BoNT toxicity can be selected, for example, depending on immunological factors, such as potency of the peptide in inducing an immune response, and technical factors, such as chemical synthesis yields. It is also understood that the two or more BoNT/A peptides can be provided separately or as part of a compound molecule such as a chimeric peptide or heterologous protein.

In an aspect of this embodiment, a method of preventing or reducing BoNT toxicity uses two or more immunoreactive BoNT/A peptides selected from the following amino acid sequences: 533-551 of SEQ ID NO:1 (N7), 547-565 of SEQ ID NO:1 (N8), 743-761 of SEQ ID NO:1 (N22), 785-803 of SEQ ID NO:1 (N25), 813-831 of SEQ ID NO:1 (N27), 995-1013 of SEQ ID NO:1 (C11); 1051-1069 of SEQ ID NO:1 (C15), 1177-1195 of SEQ ID NO:1 (C24), and 1275-1296 of SEQ ID NO:1 (C31), or a conservative variant, a non-conservative variant or an immunoreactive fragment thereof, with the proviso that the BoNT/A peptide is not SEQ ID NO:2. In another aspect of this embodiment, one of the selected amino acid sequence is 533-551 of SEQ ID NO:1 (N8) or a conservative variant, a non-conservative variant or an immunoreactive fragment thereof, with the proviso that the BoNT/A peptide is not SEQ ID NO:2. In yet another aspect of this embodiment, the following two amino acid sequences are selected: 533-551 of SEQ ID NO:1 (N8) and 981-999 of SEQ ID NO:1 (C10), or a conservative variant, a non-conservative variant or an immunoreactive fragment thereof, with the proviso that the BoNT/A peptide is not SEQ ID NO:2. In yet another aspect of this embodiment, the following three amino acid sequences are selected: 533-551 of SEQ ID NO:1 (N8), 981-999 of SEQ ID NO:1 (C10) and 1051-1069 of SEQ ID NO:1 (C15), or a conservative variant, a non-conservative variant or an immunoreactive fragment thereof, with the proviso that the BoNT/A peptide is not SEQ ID NO:2. In a further aspect of this embodiment, one of the amino acid sequences selected is 785-803 of SEQ ID NO:1 (N25) or a conservative variant, a non-conservative variant or an immunoreactive fragment thereof, with the proviso that the BoNT/A peptide is not SEQ ID NO:2. In a still further aspect of this embodiment, the following two amino acid sequences are selected: 785-803 of SEQ ID NO:1 (N25) and 981-999 of SEQ ID NO:1 (C10), or a conservative variant, a non-conservative variant or an immunoreactive fragment thereof, with the proviso that the BoNT/A peptide is not SEQ ID NO:2. In a still further aspect of this embodiment, the following three amino acid sequences are selected: 785-803 of SEQ ID NO:1 (N25), 981-999 of SEQ ID NO:1 (C10) and 1051-1069 of SEQ ID NO:1 (C15), or a conservative variant, a non-conservative variant or an immunoreactive fragment thereof, with the proviso that the BoNT/A peptide is not SEQ ID NO:2. In an additional aspect of this embodiment, one of the amino acid sequences selected is 813-831 of SEQ ID NO:1 (N27) or a conservative variant, a non-conservative variant or an immunoreactive fragment thereof, with the proviso that the BoNT/A peptide is not SEQ ID NO:2. In another additional aspect of this embodiment, the following two amino acid sequences are selected: 813-831 of SEQ ID NO:1 (N27) and 981-999 of SEQ ID NO:1 (C10), or a conservative variant, a non-conservative variant or an immunoreactive fragment thereof, with the proviso that the BoNT/A peptide is not SEQ ID NO:2. In another additional aspect of this embodiment, the following three amino acid sequences are selected: 813-831 of SEQ ID NO:1 (N27), 981-999 of SEQ ID NO:1 (C10) and 1051-1069 of SEQ ID NO:1 (C15), or a conservative variant, a non-conservative variant or an immunoreactive fragment thereof, with the proviso that the BoNT/A peptide is not SEQ ID NO:2.

In an aspect of this embodiment, a method of preventing or reducing BoNT toxicity uses two or more immunoreactive BoNT/A peptides selected from the following amino acid sequences: 659-677 of SEQ ID NO:1 (N16), 729-747 of SEQ ID NO:1 (N21), 799-817 of SEQ ID NO:1 (N26), 1065-1083 of SEQ ID NO:1 (C16), 1163-1181 of SEQ ID NO:1 (C23), and 1275-1296 of SEQ ID NO:1 (C31), or a conservative variant, a non-conservative variant or an immunoreactive fragment thereof, with the proviso that the BoNT/A peptide is not SEQ ID NO:2. In another aspect of this embodiment, one of the amino acid sequences selected is 1065-1083 of SEQ ID NO:1 (C16) or a conservative variant, a non-conservative variant or an immunoreactive fragment thereof, with the proviso that the BoNT/A peptide is not SEQ ID NO:2. In yet another aspect of this embodiment, the following two amino acid sequences are selected: 1065-1083 of SEQ ID NO:1 (C16) and 1163-1181 of SEQ ID NO:1 (C23), or a conservative variant, a non-conservative variant or an immunoreactive fragment thereof, with the proviso that the BoNT/A peptide is not SEQ ID NO:2. In yet another aspect of this embodiment, the following three amino acid sequences are selected: 1065-1083 of SEQ ID NO:1 (C16), 1163-1181 of SEQ ID NO:1 (C23) and 1275-1296 of SEQ ID NO:1 (C31), or a conservative variant, a non-conservative variant or an immunoreactive fragment thereof, with the proviso that the BoNT/A peptide is not SEQ ID NO:2. In a further aspect of this embodiment, one of the amino acid sequences selected is 799-817 of SEQ ID NO:1 (N26) or a conservative variant, a non-conservative variant or an immunoreactive fragment thereof, with the proviso that the BoNT/A peptide is not SEQ ID NO:2. In a still further aspect of this embodiment, the following two amino acid sequences are selected: 799-817 of SEQ ID NO:1 (N26) and 1065-1083 of SEQ ID NO:1 (C16), or a conservative variant, a non-conservative variant or an immunoreactive fragment thereof, with the proviso that the BoNT/A peptide is not SEQ ID NO:2. In a still further aspect of this embodiment, the following three amino acid sequences are selected: 799-817 of SEQ ID NO:1 (N26), 1065-1083 of SEQ ID NO:1 (C16) and 1163-1181 of SEQ ID NO:1 (C23), or a conservative variant, a non-conservative variant or an immunoreactive fragment thereof, with the proviso that the BoNT/A peptide is not SEQ ID NO:2. In an additional aspect of this embodiment, one of the amino acid sequences selected is 729-747 of SEQ ID NO:1 (N21) or a conservative variant, a non-conservative variant or an immunoreactive fragment thereof, with the proviso that the BoNT/A peptide is not SEQ ID NO:2. In another additional aspect of this embodiment, the following two amino acid sequences are selected: 729-747 of SEQ ID NO:1 (N21) and 1065-1083 of SEQ ID NO:1 (C16), or a conservative variant, a non-conservative variant or an immunoreactive fragment thereof, with the proviso that the BoNT/A peptide is not SEQ ID NO:2. In another additional aspect of this embodiment, the following three amino acid sequences are selected: 729-747 of SEQ ID NO:1 (N21), 1065-1083 of SEQ ID NO:1 (C16) and 1163-1181 of SEQ ID NO:1 (C23), or a conservative variant, a non-conservative variant or an immunoreactive fragment thereof, with the proviso that the BoNT/A peptide is not SEQ ID NO:2.

It is also envisioned that any and all combinations of BoNT/A peptides disclosed in the specification can be useful for preventing or reducing BoNT toxicity, including, e.g., BoNT/A peptides of SEQ ID NO:1, BoNT/A conservative variants, BoNT/A non-conservative variants and BoNT/A immunoreactive fragments. Thus, aspects of this embodiment include one or more BoNT/A peptides comprising one or more BoNT/A peptides of SEQ ID NO: 1 and one or more BoNT/A conservative variants; one or more BoNT/A peptides of SEQ ID NO: 1 and one or more BoNT/A non-conservative variants; one or more BoNT/A peptides of SEQ ID NO: 1 and one or more BoNT/A immunoreactive fragments; one or more BoNT/A conservative variants and one or more BoNT/A non-conservative variants; one or more BoNT/A conservative variants and one or more BoNT/A immunoreactive fragments; one or more BoNT/A non-conservative variants and one or more BoNT/A immunoreactive fragments; one or more BoNT/A peptides of SEQ ID NO: 1, one or more BoNT/A conservative variants and one or more BoNT/A non-conservative variants; one or more BoNT/A peptides of SEQ ID NO: 1, one or more BoNT/A conservative variants and one or more BoNT/A immunoreactive fragments; one or more BoNT/A peptides of SEQ ID NO: 1, one or more BoNT/A non-conservative variants and one or more BoNT/A immunoreactive fragments; one or more BoNT/A conservative variants, one or more BoNT/A non-conservative variants and one or more BoNT/A immunoreactive fragments; or one or more BoNT/A peptides of SEQ ID NO: 1, one or more BoNT/A conservative variants, one or more BoNT/A non-conservative variants and one or more BoNT/A immunoreactive fragments.

One skilled in the art can determine if a BoNT/A vaccine induces an immune response, as methods for detecting immune responses are well known in the art. Non-limiting examples involve measuring the titer of BoNT/A-selective antibodies in an animal primed with the vaccine and boosted with the antigen, or determining the presence of antibodies in the blood of an immunized animal that are cross-reactive with the antigen by ELISA, Western blotting or other well-known methods. Cell-mediated immune responses can be determined, for example, by measuring cytotoxic T cell response to antigen using a variety of methods described hereinabove or well known in the art.

A vaccine composition useful in a method of the invention can be administered by any of a variety of routes, as described below. Those skilled in the art can readily determine for a particular BoNT/A vaccine, the appropriate antigen payload; route of immunization; volume of dose; and vaccination regimen useful in a particular animal, for example, humans.

As disclosed herein a vaccine composition is administered to a human or other mammal to treat a condition characterized by BoNT/A immunoresistance. As used herein, the term “treating,” when used in reference to administering to a human or other mammal an effective amount of a vaccine composition, means reducing a symptom of a condition characterized by BoNT/A toxicity, or delaying or preventing onset of a symptom of a condition characterized by BoNT/A toxicity in the human or other mammal. For example, the term “treating” can mean reducing a symptom of a condition characterized by BoNT/A toxicity by at least 30%, 40%, 60%, 70%, 80%, 90% or 100%. The effectiveness of a vaccine composition in treating a condition characterized by BoNT/A toxicity can be determined by observing one or more clinical symptoms or physiological indicators associated with the condition. An improvement in a condition characterized by BoNT/A toxicity also can be indicated by a reduced need for a concurrent therapy. Those of skill in the art will know the appropriate symptoms or indicators associated with specific conditions and will know how to determine if a human or other mammal is a candidate for treatment with a vaccine composition disclosed in the present specification. In particular, it is understood that those skilled in the art will be able to determine if a condition if characterized by BoNT/A toxicity, for example, by comparison of levels of BoNT/A toxicity from a normal control human or other mammal.

The appropriate effective amount to be administered for a particular application of the methods can be determined by those skilled in the art, using the guidance provided herein. For example, an effective amount can be extrapolated from in vitro and in vivo assays as described herein above. One skilled in the art will recognize that the condition of the patient can be monitored throughout the course of therapy and that the effective amount of a vaccine composition that is administered can be adjusted accordingly.

A vaccine composition useful in the invention generally is administered in a pharmaceutical acceptable composition. As used herein, the term “pharmaceutically acceptable” refer to any molecular entity or composition that does not produce an adverse, allergic or other untoward or unwanted reaction when administered to a human or other mammal. As used herein, the term “pharmaceutically acceptable composition” refers to a therapeutically effective concentration of an active ingredient. A pharmaceutical composition may be administered to a patient alone, or in combination with other supplementary active ingredients, agents, drugs or hormones. The pharmaceutical compositions may be manufactured using any of a variety of processes, including, without limitation, conventional mixing, dissolving, granulating, dragee-making, levigating, emulsifying, encapsulating, entrapping, and lyophilizing. The pharmaceutical composition can take any of a variety of forms including, without limitation, a sterile solution, suspension, emulsion, lyophilizate, tablet, pill, pellet, capsule, powder, syrup, elixir or any other dosage form suitable for administration.

It is also envisioned that a pharmaceutical composition disclosed in the present specification can optionally include a pharmaceutically acceptable carriers that facilitate processing of an active ingredient into pharmaceutically acceptable compositions. As used herein, the term “pharmacologically acceptable carrier” refers to any carrier that has substantially no long term or permanent detrimental effect when administered and encompasses terms such as “pharmacologically acceptable vehicle, stabilizer, diluent, auxiliary or excipient.” Such a carrier generally is mixed with an active compound, or permitted to dilute or enclose the active compound and can be a solid, semi-solid, or liquid agent. It is understood that the active ingredients can be soluble or can be delivered as a suspension in the desired carrier or diluent. Any of a variety of pharmaceutically acceptable carriers can be used including, without limitation, aqueous media such as, e.g., distilled, deionized water, saline; solvents; dispersion media; coatings; antibacterial and antifungal agents; isotonic and absorption delaying agents; or any other inactive ingredient. Selection of a pharmacologically acceptable carrier can depend on the mode of administration. Except insofar as any pharmacologically acceptable carrier is incompatible with the active ingredient, its use in pharmaceutically acceptable compositions is contemplated. Non-limiting examples of specific uses of such pharmaceutical carriers can be found in PHARMACEUTICAL DOSAGE FORMS AND DRUG DELIVERY SYSTEMS (Howard C. Ansel et al., eds., Lippincott Williams & Wilkins Publishers, 7^{th} ed. 1999); REMINGTON: THE SCIENCE AND PRACTICE OF PHARMACY (Alfonso R. Gennaro ed., Lippincott, Williams & Wilkins, 20^{th} ed. 2000); GOODMAN & GILMAN'S THE PHARMACOLOGICAL BASIS OF THERAPEUTICS (Joel G. Hardman et al., eds., McGraw-Hill Professional, 10^{th} ed. 2001); and HANDBOOK OF PHARMACEUTICAL EXCIPIENTS (Raymond C. Rowe et al., APhA Publications, 4^{th} edition 2003) which are hereby incorporated by reference in their entirety. These protocols are routine procedures and any modifications are well within the scope of one skilled in the art and from the teaching herein.

It is further envisioned that a pharmaceutical composition disclosed in the present specification can optionally include, without limitation, other pharmaceutically acceptable components, including, without limitation, buffers, preservatives, tonicity adjusters, salts, antioxidants, physiological substances, pharmacological substances, bulking agents, emulsifying agents, wetting agents, sweetening or flavoring agents, and the like. Various buffers and means for adjusting pH can be used to prepare a pharmaceutical composition disclosed in the present specification, provided that the resulting preparation is pharmaceutically acceptable. Such buffers include, without limitation, acetate buffers, citrate buffers, phosphate buffers, neutral buffered saline, phosphate buffered saline and borate buffers. It is understood that acids or bases can be used to adjust the pH of a composition as needed. Pharmaceutically acceptable antioxidants include, without limitation, sodium metabisulfite, sodium thiosulfate, acetylcysteine, butylated hydroxyanisole and butylated hydroxytoluene. Useful preservatives include, without limitation, benzalkonium chloride, chlorobutanol, thimerosal, phenylmercuric acetate, phenylmercuric nitrate and a stabilized oxy chloro composition, for example, PURITE®. Tonicity adjustors useful in a pharmaceutical composition include, without limitation, salts such as, e.g., sodium chloride, potassium chloride, mannitol or glycerin and other pharmaceutically acceptable tonicity adjustor. The pharmaceutical composition may be provided as a salt and can be formed with many acids, including but not limited to, hydrochloric, sulfuric, acetic, lactic, tartaric, malic, succinic, etc. Salts tend to be more soluble in aqueous or other protonic solvents than are the corresponding free base forms. It is understood that these and other substances known in the art of pharmacology can be included in a pharmaceutical composition useful in the invention.

A vaccine composition useful in a method of the invention is administered to a human or other mammal in an effective amount. Such an effective amount generally is the minimum dose necessary to achieve the desired therapeutic effect, which can be, for example, that amount roughly necessary to reduce the symptoms associated with BoNT/A toxicity. For example, the term “effective amount” when used with respect to treating BoNT/A toxicity can be a dose sufficient to the symptoms, for example, by at least 30%, 40%, 50%, 60%, 70%, 80%, 90% or 100%. Such a dose generally is in the range of 0.1-1000 mg/day and can be, for example, in the range of 0.1-500 mg/day, 0.5-500 mg/day, 0.5-100 mg/day, 0.5-50 mg/day, 0.5-20 mg/day, 0.5-10 mg/day or 0.5-5 mg/day, with the actual amount to be administered determined by a physician taking into account the relevant circumstances including the severity of the BoNT/A toxicity, the age and weight of the patient, the patient's general physical condition, the vaccine composition, the cause of the BoNT/A toxicity and the route of administration. Where repeated administration is used, the frequency of administration depends, in part, on the half-life of the vaccine composition. Suppositories and extended release formulations can be useful in the invention and include, for example, dermal patches, formulations for deposit on or under the skin and formulations for intramuscular injection. It is understood that slow-release formulations also can be useful in the methods of the invention. The subject receiving the vaccine composition can be any mammal or other vertebrate capable of experiencing BoNT/A toxicity, for example, a human, primate, horse, cow, dog, cat or bird.

Various routes of administration can be useful for treating BoNT/A toxicity, according to a method of the invention. A pharmaceutical composition useful in the methods of the invention can be administered to a mammal by any of a variety of means depending, for example, on the type and location of BoNT/A toxicity to be treated, the vaccine composition or other compound to be included in the composition, and the history, risk factors and symptoms of the subject. Routes of administration suitable for the methods of the invention include both systemic and local administration. As non-limiting examples, a pharmaceutical composition useful for treating BoNT/A toxicity can be administered orally or by subcutaneous pump; by dermal patch; by intravenous, subcutaneous or intramuscular injection; by topical drops, creams, gels or ointments; as an implanted or injected extended release formulation; as a bioerodible or non-bioerodible delivery system; by subcutaneous minipump or other implanted device; by intrathecal pump or injection; or by epidural injection. An exemplary list of biodegradable polymers and methods of use are described in, e.g., HANDBOOK OF BIODEGRADABLE POLYMERS (Abraham J. Domb et al., eds., Overseas Publishers Association, 1997); CONTROLLED DRUG DELIVERY: DESIGNING TECHNOLOGIES FOR THE FUTURE (Kinam Park & Randy J. Mrsny eds., American Chemical Association, 2000); Vernon G. Wong, Method for Reducing or Preventing Transplant Rejection in the Eye and Intraocular Implants for Use Therefor, U.S. Pat. No. 6,699,493 (Mar. 2, 2004); Vernon G. Wong & Mae W. L. Hu, Methods for Treating Inflammation-mediated Conditions of the Eye, U.S. Pat. No. 6,726,918 (Apr. 27, 2004); David A. Weber et al., Methods and Apparatus for Delivery of Ocular Implants, U.S. Patent Publication No. US2004/0054374 (Mar. 18, 2004); Thierry Nivaggioli et al., Biodegradable Ocular Implant, U.S. Patent Publication No. US2004/0137059 (Jul. 15, 2004), which are hereby incorporated by reference in their entirety. It is understood that the frequency and duration of dosing will be dependent, in part, on the relief desired and the half-life of the BoNT/A toxicity.

In particular embodiments, a method of the invention is practiced by peripheral administration of a vaccine composition. As used herein, the term “peripheral administration” or “administered peripherally” means introducing an agent into a subject outside of the central nervous system. Peripheral administration encompasses any route of administration other than direct administration to the spine or brain. As such, it is clear that intrathecal and epidural administration as well as cranial injection or implantation are not within the scope of the term “peripheral administration” or “administered peripherally.”

Peripheral administration can be local or systemic. Local administration results in significantly more of a pharmaceutical composition being delivered to and about the site of local administration than to regions distal to the site of administration. Systemic administration results in delivery of a pharmaceutical composition to essentially the entire peripheral nervous system of the subject and may also result in delivery to the central nervous system depending on the properties of the composition.

Routes of peripheral administration useful in the methods of the invention encompass, without limitation, oral administration, topical administration, intravenous or other injection, and implanted minipumps or other extended release devices or formulations. A pharmaceutical composition useful in the invention can be peripherally administered, for example, orally in any acceptable form such as in a tablet, liquid, capsule, powder, or the like; by intravenous, intraperitoneal, intramuscular, subcutaneous or parenteral injection; by transdermal diffusion or electrophoresis; topically in any acceptable form such as in drops, creams, gels or ointments; and by minipump or other implanted extended release device or formulation.

IX. Method of Preparing Anti-BoNT/A Antibody Compositions

A BoNT/A peptide composition disclosed in the present specification can be used in a process for preparing an anti-BoNT antibody composition. Thus, the present invention provides a method of preparing an anti-BoNT/A antibody by administering to an animal a BoNT/A peptide disclosed in the present specification; collecting from the animal a sample containing an antibody or antibody-producing cell; and processing the sample to isolate the anti-BoNT/A antibody, with the proviso that the BoNT/A peptide is not SEQ ID NO:2. Antibodies to be prepared according to a method of the invention include polyclonal and monoclonal antibodies. An anti-BoNT/A antibody prepared according to a method of the invention, or a monoclonal anti-BoNT/A antibody of the invention as described further below, can be used in a variety of applications. Such applications include, for example, detection of botulinum toxin in a sample, such as a substance suspected to be contaminated with BoNT/A.

In an embodiment of the present invention, a BoNT/A peptide useful in a method disclosed in the present specification for preparing an anti-BoNT/A antibody composition has a length of at most 60 amino acids and consisting of at least 5 contiguous amino acids selected from one of the following BoNT/A amino acid sequences: 449-467 of SEQ ID NO:1(N1), 463-481 of SEQ ID NO:1 (N2), 491-509 of SEQ ID NO:1 (N4), 505-523 of SEQ ID NO:1 (N5), 519-537 of SEQ ID NO:1 (N6), 533-551 of SEQ ID NO:1 (N7), 547-565 of SEQ ID NO:1 (N8), 561-579 of SEQ ID NO:1 (N9), 575-593 of SEQ ID NO:1 (N10), 589-607 of SEQ ID NO:1 (N11), 631-649 of SEQ ID NO:1 (N14), 645-663 of SEQ ID NO:1 (N15), 659-677 of SEQ ID NO:1 (N16), 673-691 of SEQ ID NO:1 (N17), 701-719 of SEQ ID NO:1 (N19), 715-733 of SEQ ID NO:1 (N20), 729-747 of SEQ ID NO:1 (N21), 743-761 of SEQ ID NO:1 (N22), 757-775 of SEQ ID NO:1 (N23), 771-789 of SEQ ID NO:1 (N24), 785-803 of SEQ ID NO:1 (N25), 799-817 of SEQ ID NO:1 (N26), 813-831 of SEQ ID NO:1 (N27), 827-845 of SEQ ID NO:1 (N28), 869-887 of SEQ ID NO:1 (C2), 883-901 of SEQ ID NO:1 (C3), 911-929 of SEQ ID NO:1 (C5), 925-943 of SEQ ID NO:1 (C6), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1065-1083 of SEQ ID NO:1 (C16), 1079-1097 of SEQ ID NO:1 (C17), 1107-1125 of SEQ ID NO:1 (C19), 1121-1139 of SEQ ID NO:1 (C20), 1135-1153 of SEQ ID NO:1 (C21), 1149-1167 of SEQ ID NO:1 (C22), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28), 1247-1265 of SEQ ID NO:1 (C29), 1261-1279 of SEQ ID NO:1 (C30), or 1275-1296 of SEQ ID NO:1 (C31), or a conservative variant or immunoreactive fragment thereof, with the proviso that the BoNT/A peptide is not SEQ ID NO:2.

In another embodiment of the present invention, a BoNT/A peptide useful in a method disclosed in the present specification for preparing an anti-BoNT/A antibody composition has a length of at most 60 amino acids and consisting of at least 5 contiguous amino acids selected from one of the following BoNT/A amino acid sequences: 449-467 of SEQ ID NO:1(N1), 463-481 of SEQ ID NO:1 (N2), 491-509 of SEQ ID NO:1 (N4), 505-523 of SEQ ID NO:1 (N5), 519-537 of SEQ ID NO:1 (N6), 533-551 of SEQ ID NO:1 (N7), 547-565 of SEQ ID NO:1 (N8), 561-579 of SEQ ID NO:1 (N9), 575-593 of SEQ ID NO:1 (N10), 589-607 of SEQ ID NO:1 (N11), 631-649 of SEQ ID NO:1 (N14), 645-663 of SEQ ID NO:1 (N15), 659-677 of SEQ ID NO:1 (N16), 673-691 of SEQ ID NO:1 (N17), 701-719 of SEQ ID NO:1 (N19), 715-733 of SEQ ID NO:1 (N20), 729-747 of SEQ ID NO:1 (N21), 743-761 of SEQ ID NO:1 (N22), 757-775 of SEQ ID NO:1 (N23), 771-789 of SEQ ID NO:1 (N24), 785-803 of SEQ ID NO:1 (N25), 799-817 of SEQ ID NO:1 (N26), 813-831 of SEQ ID NO:1 (N27), 827-845 of SEQ ID NO:1 (N28), 869-887 of SEQ ID NO:1 (C2), 883-901 of SEQ ID NO:1 (C3), 911-929 of SEQ ID NO:1 (C5), 925-943 of SEQ ID NO:1 (C6), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1065-1083 of SEQ ID NO:1 (C16), 1079-1097 of SEQ ID NO:1 (C17), 1107-1125 of SEQ ID NO:1 (C19), 1121-1139 of SEQ ID NO:1 (C20), 1135-1153 of SEQ ID NO:1 (C21), 1149-1167 of SEQ ID NO:1 (C22), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28), 1247-1265 of SEQ ID NO:1 (C29), 1261-1279 of SEQ ID NO:1 (C30), or 1275-1296 of SEQ ID NO:1 (C31). In an aspect of this embodiment, such a BoNT/A peptide is selected from one of the following amino acid sequences: 463-481 of SEQ ID NO:1 (N2), 519-537 of SEQ ID NO:1 (N6), 533-551 of SEQ ID NO:1 (N7), 547-565 of SEQ ID NO:1 (N8), 561-579 of SEQ ID NO:1 (N9), 589-607 of SEQ ID NO:1 (N11), 659-677 of SEQ ID NO:1 (N16), 743-761 of SEQ ID NO:1 (N22), 785-803 of SEQ ID NO:1 (N25), 813-831 of SEQ ID NO:1 (N27), 827-845 of SEQ ID NO:1 (N28), 869-887 of SEQ ID NO:1 (C2), 883-901 of SEQ ID NO:1 (C3), 911-929 of SEQ ID NO:1 (C6), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1121-1139 of SEQ ID NO:1 (C20), 1135-1153 of SEQ ID NO:1 (C21), 1177-1195 of SEQ ID NO:1 (C24), 1261-1279 of SEQ ID NO:1 (C30) or 1275-1296 of SEQ ID NO:1 (C31). In another aspect of this embodiment, such a BoNT/A peptide is selected from one of the following amino acid sequences: 463-481 of SEQ ID NO:1 (N2), 533-551 of SEQ ID NO:1 (N7), 547-565 of SEQ ID NO:1 (N8), 561-579 of SEQ ID NO:1 (N9), 659-677 of SEQ ID NO:1 (N16), 743-761 of SEQ ID NO:1 (N22), 785-803 of SEQ ID NO:1 (N25), 813-831 of SEQ ID NO:1 (N27), 883-901 of SEQ ID NO:1 (C3), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1121-1139 of SEQ ID NO:1 (C20) or 1177-1195 of SEQ ID NO:1 (C24) or 1275-1296 of SEQ ID NO: 1 (C31). In another aspect of this embodiment, such a BoNT/A peptide is selected from one of the following amino acid sequences: 533-551 of SEQ ID NO: 1 (N7), 547-565 of SEQ ID NO: 1 (N8), 743-761 of SEQ ID NO: 1 (N22), 785-803 of SEQ ID NO: 1 (N25), 813-831 of SEQ ID NO: 1 (N27), 995-1013 of SEQ ID NO: 1 (C11); 1051-1069 of SEQ ID NO: 1 (C15), 1177-1195 of SEQ ID NO: 1 (C24), or 1275-1296 of SEQ ID NO: 1 (C31).

In yet another aspect of this embodiment, a BoNT/A peptide useful in a method disclosed in the present specification for preparing an anti-BoNT/A antibody composition has a length of at most 60 amino acids and consisting of at least 5 contiguous amino acids selected from one of the following BoNT/A amino acid sequences: 463-481 of SEQ ID NO:1 (N2), 505-523 of SEQ ID NO:1 (N5), 519-537 of SEQ ID NO:1 (N6), 533-551 of SEQ ID NO:1 (N7), 603-621 of SEQ ID NO:1 (N12), 645-663 of SEQ ID NO:1 (N15), 659-677 of SEQ ID NO:1 (N16), 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1079-1097 of SEQ ID NO:1 (C17), 1107-1125 of SEQ ID NO:1 (C19), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28) or 1275-1296 of SEQ ID NO:1 (C31). In yet another aspect of this embodiment, such a BoNT/A peptide is selected from one of the following amino acid sequences: 533-551 of SEQ ID NO:1 (N7), 659-677 of SEQ ID NO:1 (N16), 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1107-1125 of SEQ ID NO:1 (C19), 1163-1181 of SEQ ID NO:1 (C23), 1191-1209 of SEQ ID NO:1 (C25), 1233-1251 of SEQ ID NO:1 (C28) or 1275-1296 of SEQ ID NO:1 (C31). In yet another aspect of this embodiment, such a BoNT/A peptide is selected from one of the following amino acid sequences: 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1079-1097 of SEQ ID NO:1 (C17), 1107-1125 of SEQ ID NO:1 (C19), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28) or 1275-1296 of SEQ ID NO:1 (C31). In yet another aspect of this embodiment, such a BoNT/A peptide is selected from one of the following amino acid sequences: 729-747 of SEQ ID NO:1 (N21) 799-817 of SEQ ID NO:1 (N26), 1065-1083 of SEQ ID NO:1 (C16), 1163-1181 of SEQ ID NO:1 (C23) or 1275-1296 of SEQ ID NO:1 (C31).

In yet another embodiment of the present invention, a BoNT/A peptide useful in a method disclosed in the present specification for preparing an anti-BoNT/A antibody composition is selected from one of the following BoNT/A amino acid sequences: 449-467 of SEQ ID NO:1(N1), 463-481 of SEQ ID NO:1 (N2), 491-509 of SEQ ID NO:1 (N4), 505-523 of SEQ ID NO:1 (N5), 519-537 of SEQ ID NO:1 (N6), 533-551 of SEQ ID NO:1 (N7), 547-565 of SEQ ID NO:1 (N8), 561-579 of SEQ ID NO:1 (N9), 575-593 of SEQ ID NO:1 (N10), 589-607 of SEQ ID NO:1 (N11), 631-649 of SEQ ID NO:1 (N14), 645-663 of SEQ ID NO:1 (N15), 659-677 of SEQ ID NO:1 (N16), 673-691 of SEQ ID NO:1 (N17), 701-719 of SEQ ID NO:1 (N19), 715-733 of SEQ ID NO:1 (N20), 729-747 of SEQ ID NO:1 (N21), 743-761 of SEQ ID NO:1 (N22), 757-775 of SEQ ID NO:1 (N23), 771-789 of SEQ ID NO:1 (N24), 785-803 of SEQ ID NO:1 (N25), 799-817 of SEQ ID NO:1 (N26), 813-831 of SEQ ID NO:1 (N27), 827-845 of SEQ ID NO:1 (N28), 869-887 of SEQ ID NO:1 (C2), 883-901 of SEQ ID NO:1 (C3), 911-929 of SEQ ID NO:1 (C5), 925-943 of SEQ ID NO:1 (C6), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1065-1083 of SEQ ID NO:1 (C16), 1079-1097 of SEQ ID NO:1 (C17), 1107-1125 of SEQ ID NO:1 (C19), 1121-1139 of SEQ ID NO:1 (C20), 1135-1153 of SEQ ID NO:1 (C21), 1149-1167 of SEQ ID NO:1 (C22), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28), 1247-1265 of SEQ ID NO:1 (C29), 1261-1279 of SEQ ID NO:1 (C30), or 1275-1296 of SEQ ID NO:1 (C31). In an aspect of this embodiment, such a BoNT/A peptide is selected from one of the following amino acid sequences: 463-481 of SEQ ID NO:1 (N2), 519-537 of SEQ ID NO:1 (N6), 533-551 of SEQ ID NO:1 (N7), 547-565 of SEQ ID NO:1 (N8), 561-579 of SEQ ID NO:1 (N9), 589-607 of SEQ ID NO:1 (N11), 659-677 of SEQ ID NO:1 (N16), 743-761 of SEQ ID NO:1 (N22), 785-803 of SEQ ID NO:1 (N25), 813-831 of SEQ ID NO:1 (N27), 827-845 of SEQ ID NO:1 (N28), 869-887 of SEQ ID NO:1 (C2), 883-901 of SEQ ID NO:1 (C3), 911-929 of SEQ ID NO:1 (C6), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1121-1139 of SEQ ID NO:1 (C20), 1135-1153 of SEQ ID NO:1 (C21), 1177-1195 of SEQ ID NO:1 (C24), 1261-1279 of SEQ ID NO:1 (C30) or 1275-1296 of SEQ ID NO:1 (C31). In another aspect of this embodiment, such a BoNT/A peptide is selected from one of the following amino acid sequences: 463-481 of SEQ ID NO:1 (N2), 533-551 of SEQ ID NO:1 (N7), 547-565 of SEQ ID NO:1 (N8), 561-579 of SEQ ID NO:1 (N9), 659-677 of SEQ ID NO:1 (N16), 743-761 of SEQ ID NO:1 (N22), 785-803 of SEQ ID NO:1 (N25), 813-831 of SEQ ID NO:1 (N27), 883-901 of SEQ ID NO:1 (C3), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1121-1139 of SEQ ID NO:1 (C20) or 1177-1195 of SEQ ID NO:1 (C24) or 1275-1296 of SEQ ID NO: 1 (C31). In another aspect of this embodiment, such a BoNT/A peptide is selected from one of the following amino acid sequences: 533-551 of SEQ ID NO: 1 (N7), 547-565 of SEQ ID NO: 1 (N8), 743-761 of SEQ ID NO: 1 (N22), 785-803 of SEQ ID NO: 1 (N25), 813-831 of SEQ ID NO: 1 (N27), 995-1013 of SEQ ID NO: 1 (C11); 1051-1069 of SEQ ID NO: 1 (C15), 1177-1195 of SEQ ID NO: 1 (C24), or 1275-1296 of SEQ ID NO: 1 (C31).

In yet another aspect of this embodiment, a BoNT/A peptide useful in a method disclosed in the present specification for preparing an anti-BoNT/A antibody composition is selected from one of the following BoNT/A amino acid sequences: 463-481 of SEQ ID NO:1 (N2), 505-523 of SEQ ID NO:1 (N5), 519-537 of SEQ ID NO:1 (N6), 533-551 of SEQ ID NO:1 (N7), 603-621 of SEQ ID NO:1 (N12), 645-663 of SEQ ID NO:1 (N15), 659-677 of SEQ ID NO:1 (N16), 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1079-1097 of SEQ ID NO:1 (C17), 1107-1125 of SEQ ID NO:1 (C19), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28) or 1275-1296 of SEQ ID NO:1 (C31). In yet another aspect of this embodiment, such a BoNT/A peptide is selected from one of the following amino acid sequences: 533-551 of SEQ ID NO:1 (N7), 659-677 of SEQ ID NO:1 (N16), 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1107-1125 of SEQ ID NO:1 (C19), 1163-1181 of SEQ ID NO:1 (C23), 1191-1209 of SEQ ID NO:1 (C25), 1233-1251 of SEQ ID NO:1 (C28) or 1275-1296 of SEQ ID NO:1 (C31). In yet another aspect of this embodiment, such a BoNT/A peptide is selected from one of the following amino acid sequences: 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1079-1097 of SEQ ID NO:1 (C17), 1107-1125 of SEQ ID NO:1 (C19), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28) or 1275-1296 of SEQ ID NO:1 (C31). In yet another aspect of this embodiment, such a BoNT/A peptide is selected from one of the following amino acid sequences: 729-747 of SEQ ID NO:1 (N21) 799-817 of SEQ ID NO:1 (N26), 1065-1083 of SEQ ID NO:1 (C16), 1163-1181 of SEQ ID NO:1 (C23) or 1275-1296 of SEQ ID NO:1 (C31).

In yet another embodiment of the present invention, a BoNT/A peptide useful in a method disclosed in the present specification for preparing an anti-BoNT/A antibody composition has a length of at most 60 amino acids and consisting of at least 5 contiguous amino acids selected from one of the following BoNT/A amino acid sequences: 449-467 of SEQ ID NO:1(N1), 463-481 of SEQ ID NO:1 (N2), 491-509 of SEQ ID NO:1 (N4), 505-523 of SEQ ID NO:1 (N5), 519-537 of SEQ ID NO:1 (N6), 533-551 of SEQ ID NO:1 (N7), 547-565 of SEQ ID NO:1 (N8), 561-579 of SEQ ID NO:1 (N9), 575-593 of SEQ ID NO:1 (N10), 589-607 of SEQ ID NO:1 (N11), 631-649 of SEQ ID NO:1 (N14), 645-663 of SEQ ID NO:1 (N15), 659-677 of SEQ ID NO:1 (N16), 673-691 of SEQ ID NO:1 (N17), 701-719 of SEQ ID NO:1 (N19), 715-733 of SEQ ID NO:1 (N20), 729-747 of SEQ ID NO:1 (N21), 743-761 of SEQ ID NO:1 (N22), 757-775 of SEQ ID NO:1 (N23), 771-789 of SEQ ID NO:1 (N24), 785-803 of SEQ ID NO:1 (N25), 799-817 of SEQ ID NO:1 (N26), 813-831 of SEQ ID NO:1 (N27), 827-845 of SEQ ID NO:1 (N28), 869-887 of SEQ ID NO:1 (C2), 883-901 of SEQ ID NO:1 (C3), 911-929 of SEQ ID NO:1 (C5), 925-943 of SEQ ID NO:1 (C6), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1065-1083 of SEQ ID NO:1 (C16), 1079-1097 of SEQ ID NO:1 (C17), 1107-1125 of SEQ ID NO:1 (C19), 1121-1139 of SEQ ID NO:1 (C20), 1135-1153 of SEQ ID NO:1 (C21), 1149-1167 of SEQ ID NO:1 (C22), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28), 1247-1265 of SEQ ID NO:1 (C29), 1261-1279 of SEQ ID NO:l (C30), or 1275-1296 of SEQ ID NO:1 (C31), or a conservative variant thereof. In an aspect of this embodiment, such a BoNT/A peptide is selected from one of the following amino acid sequences: 463-481 of SEQ ID NO:1 (N2), 519-537 of SEQ ID NO:1 (N6), 533-551 of SEQ ID NO:1 (N7), 547-565 of SEQ ID NO:1 (N8), 561-579 of SEQ ID NO:1 (N9), 589-607 of SEQ ID NO:1 (N11), 659-677 of SEQ ID NO:1 (N16), 743-761 of SEQ ID NO:1 (N22), 785-803 of SEQ ID NO:1 (N25), 813-831 of SEQ ID NO:1 (N27), 827-845 of SEQ ID NO:1 (N28), 869-887 of SEQ ID NO:1 (C2), 883-901 of SEQ ID NO:1 (C3), 911-929 of SEQ ID NO:1 (C6), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1121-1139 of SEQ ID NO:1 (C20), 1135-1153 of SEQ ID NO:1 (C21), 1177-1195 of SEQ ID NO:1 (C24), 1261-1279 of SEQ ID NO:1 (C30) or 1275-1296 of SEQ ID NO:1 (C31), or a conservative variant thereof. In another aspect of this embodiment, such a BoNT/A peptide is selected from one of the following amino acid sequences: 463-481 of SEQ ID NO:1 (N2), 533-551 of SEQ ID NO:1 (N7), 547-565 of SEQ ID NO:1 (N8), 561-579 of SEQ ID NO:1 (N9), 659-677 of SEQ ID NO:1 (N16), 743-761 of SEQ ID NO:1 (N22), 785-803 of SEQ ID NO:1 (N25), 813-831 of SEQ ID NO:1 (N27), 883-901 of SEQ ID NO:1 (C3), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1121-1139 of SEQ ID NO:1 (C20) or 1177-1195 of SEQ ID NO:1 (C24) or 1275-1296 of SEQ ID NO: 1 (C31), or a conservative variant thereof. In another aspect of this embodiment, such a BoNT/A peptide is selected from one of the following amino acid sequences: 533-551 of SEQ ID NO: 1 (N7), 547-565 of SEQ ID NO: 1 (N8), 743-761 of SEQ ID NO: 1 (N22), 785-803 of SEQ ID NO: 1 (N25), 813-831 of SEQ ID NO: 1 (N27), 995-1013 of SEQ ID NO: 1 (C11); 1051-1069 of SEQ ID NO: 1 (C15), 1177-1195 of SEQ ID NO: 1 (C24), or 1275-1296 of SEQ ID NO: 1 (C31), or a conservative variant thereof.

In yet another aspect of this embodiment, a BoNT/A peptide useful in a method disclosed in the present specification for preparing an anti-BoNT/A antibody composition has a length of at most 60 amino acids and consisting of at least 5 contiguous amino acids selected from one of the following BoNT/A amino acid sequences: 463-481 of SEQ ID NO:1 (N2), 505-523 of SEQ ID NO:1 (N5), 519-537 of SEQ ID NO:1 (N6), 533-551 of SEQ ID NO:1 (N7), 603-621 of SEQ ID NO:1 (N12), 645-663 of SEQ ID NO:1 (N15), 659-677 of SEQ ID NO:1 (N16), 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1079-1097 of SEQ ID NO:1 (C17), 1107-1125 of SEQ ID NO:1 (C19), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28) or 1275-1296 of SEQ ID NO:1 (C31), or a conservative variant thereof. In yet another aspect of this embodiment, such a BoNT/A peptide is selected from one of the following amino acid sequences: 533-551 of SEQ ID NO:1 (N7), 659-677 of SEQ ID NO:1 (N16), 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1107-1125 of SEQ ID NO:1 (C19), 1163-1181 of SEQ ID NO:1 (C23), 1191-1209 of SEQ ID NO:1 (C25), 1233-1251 of SEQ ID NO:1 (C28) or 1275-1296 of SEQ ID NO:1 (C31), or a conservative variant thereof. In yet another aspect of this embodiment, such a BoNT/A peptide is selected from one of the following amino acid sequences: 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1079-1097 of SEQ ID NO:1 (C17), 1107-1125 of SEQ ID NO:1 (C19), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28) or 1275-1296 of SEQ ID NO:1 (C31), or a conservative variant thereof. In yet another aspect of this embodiment, such a BoNT/A peptide is selected from one of the following amino acid sequences: 729-747 of SEQ ID NO:1 (N21) 799-817 of SEQ ID NO:1 (N26), 1065-1083 of SEQ ID NO:1 (C16), 1163-1181 of SEQ ID NO:1 (C23) or 1275-1296 of SEQ ID NO:1 (C31), or a conservative variant thereof.

In yet another embodiment of the present invention, a BoNT/A peptide useful in a method disclosed in the present specification for preparing an anti-BoNT/A antibody composition has a length of at most 60 amino acids and consisting of at least 5 contiguous amino acids selected from one of the following BoNT/A amino acid sequences: 449-467 of SEQ ID NO:1(N1), 463-481 of SEQ ID NO:1 (N2), 491-509 of SEQ ID NO:1 (N4), 505-523 of SEQ ID NO:1 (N5), 519-537 of SEQ ID NO:1 (N6), 533-551 of SEQ ID NO:1 (N7), 547-565 of SEQ ID NO:1 (N8), 561-579 of SEQ ID NO:1 (N9), 575-593 of SEQ ID NO:1 (N10), 589-607 of SEQ ID NO:1 (N11), 631-649 of SEQ ID NO:1 (N14), 645-663 of SEQ ID NO:1 (N15), 659-677 of SEQ ID NO:1 (N16), 673-691 of SEQ ID NO:1 (N17), 701-719 of SEQ ID NO:1 (N19), 715-733 of SEQ ID NO:1 (N20), 729-747 of SEQ ID NO:1 (N21), 743-761 of SEQ ID NO:1 (N22), 757-775 of SEQ ID NO:1 (N23), 771-789 of SEQ ID NO:1 (N24), 785-803 of SEQ ID NO:1 (N25), 799-817 of SEQ ID NO:1 (N26), 813-831 of SEQ ID NO:1 (N27), 827-845 of SEQ ID NO:1 (N28), 869-887 of SEQ ID NO:l (C2), 883-901 of SEQ ID NO:l (C3), 911-929 of SEQ ID NO:1 (C5), 925-943 of SEQ ID NO:1 (C6), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1065-1083 of SEQ ID NO:1 (C16), 1079-1097 of SEQ ID NO:1 (C17), 1107-1125 of SEQ ID NO:1 (C19), 1121-1139 of SEQ ID NO:1 (C20), 1135-1153 of SEQ ID NO:1 (C21), 1149-1167 of SEQ ID NO:l (C22), 1163-1181 of SEQ ID NO:l (C23), 1177-1195 of SEQ ID NO:1 (C24), 1191-1209 of SEQ ID NO:1 (C25), 1233-1251 of SEQ ID NO:1 (C28), 1247-1265 of SEQ ID NO:1 (C29), 1261-1279 of SEQ ID NO:1 (C30), or 1275-1296 of SEQ ID NO:1 (C31), non-conservative variant thereof. In an aspect of this embodiment, such a BoNT/A peptide is selected from one of the following amino acid sequences: 463-481 of SEQ ID NO:1 (N2), 519-537 of SEQ ID NO:1 (N6), 533-551 of SEQ ID NO:1 (N7), 547-565 of SEQ ID NO:1 (N8), 561-579 of SEQ ID NO:1 (N9), 589-607 of SEQ ID NO:1 (N11), 659-677 of SEQ ID NO:1 (N16), 743-761 of SEQ ID NO:1 (N22), 785-803 of SEQ ID NO:1 (N25), 813-831 of SEQ ID NO:1 (N27), 827-845 of SEQ ID NO:1 (N28), 869-887 of SEQ ID NO:1 (C2), 883-901 of SEQ ID NO:1 (C3), 911-929 of SEQ ID NO:1 (C6), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1121-1139 of SEQ ID NO:1 (C20), 1135-1153 of SEQ ID NO:1 (C21), 1177-1195 of SEQ ID NO:1 (C24), 1261-1279 of SEQ ID NO:1 (C30) or 1275-1296 of SEQ ID NO:1 (C31), or a non-conservative variant thereof. In another aspect of this embodiment, such a BoNT/A peptide is selected from one of the following amino acid sequences: 463-481 of SEQ ID NO:1 (N2), 533-551 of SEQ ID NO:1 (N7), 547-565 of SEQ ID NO:1 (N8), 561-579 of SEQ ID NO:1 (N9), 659-677 of SEQ ID NO:1 (N16), 743-761 of SEQ ID NO:1 (N22), 785-803 of SEQ ID NO:1 (N25), 813-831 of SEQ ID NO:1 (N27), 883-901 of SEQ ID NO:1 (C3), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1121-1139 of SEQ ID NO:1 (C20) or 1177-1195 of SEQ ID NO:1 (C24) or 1275-1296 of SEQ ID NO: 1 (C31), or a non-conservative variant thereof. In another aspect of this embodiment, such a BoNT/A peptide is selected from one of the following amino acid sequences: 533-551 of SEQ ID NO: 1 (N7), 547-565 of SEQ ID NO: 1 (N8), 743-761 of SEQ ID NO: 1 (N22), 785-803 of SEQ ID NO: 1 (N25), 813-831 of SEQ ID NO: 1 (N27), 995-1013 of SEQ ID NO: 1 (C11); 1051-1069 of SEQ ID NO: 1 (C15), 1177-1195 of SEQ ID NO: 1 (C24), or 1275-1296 of SEQ ID NO: 1 (C31), or a non-conservative variant thereof.

In yet another aspect of this embodiment, a BoNT/A peptide useful in a method disclosed in the present specification for preparing an anti-BoNT/A antibody composition has a length of at most 60 amino acids and consisting of at least 5 contiguous amino acids selected from one of the following BoNT/A amino acid sequences: 463-481 of SEQ ID NO:1 (N2), 505-523 of SEQ ID NO:1 (N5), 519-537 of SEQ ID NO:1 (N6), 533-551 of SEQ ID NO:1 (N7), 603-621 of SEQ ID NO:1 (N12), 645-663 of SEQ ID NO:1 (N15), 659-677 of SEQ ID NO:1 (N16), 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1079-1097 of SEQ ID NO:1 (C17), 1107-1125 of SEQ ID NO:1 (C19), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28) or 1275-1296 of SEQ ID NO:1 (C31), or a non-conservative variant thereof. In yet another aspect of this embodiment, such a BoNT/A peptide is selected from one of the following amino acid sequences: 533-551 of SEQ ID NO:1 (N7), 659-677 of SEQ ID NO:1 (N16), 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1107-1125 of SEQ ID NO:1 (C19), 1163-1181 of SEQ ID NO:1 (C23), 1191-1209 of SEQ ID NO:1 (C25), 1233-1251 of SEQ ID NO:1 (C28) or 1275-1296 of SEQ ID NO:1 (C31), or a non-conservative variant thereof. In yet another aspect of this embodiment, such a BoNT/A peptide is selected from one of the following amino acid sequences: 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1079-1097 of SEQ ID NO:1 (C17), 1107-1125 of SEQ ID NO:1 (C19), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28) or 1275-1296 of SEQ ID NO:1 (C31), or a non-conservative variant thereof. In yet another aspect of this embodiment, such a BoNT/A peptide is selected from one of the following amino acid sequences: 729-747 of SEQ ID NO:1 (N21) 799-817 of SEQ ID NO:1 (N26), 1065-1083 of SEQ ID NO:1 (C16), 1163-1181 of SEQ ID NO:1 (C23) or 1275-1296 of SEQ ID NO:1 (C31), or a non-conservative variant thereof.

In yet another embodiment of the present invention, a BoNT/A peptide useful in a method disclosed in the present specification for preparing an anti-BoNT/A antibody composition has a length of at most 60 amino acids and consisting of at least 5 contiguous amino acids selected from one of the following BoNT/A amino acid sequences: 449-467 of SEQ ID NO:1(N1), 463-481 of SEQ ID NO:1 (N2), 491-509 of SEQ ID NO:1 (N4), 505-523 of SEQ ID NO:1 (N5), 519-537 of SEQ ID NO:1 (N6), 533-551 of SEQ ID NO:1 (N7), 547-565 of SEQ ID NO:1 (N8), 561-579 of SEQ ID NO:1 (N9), 575-593 of SEQ ID NO:1 (N10), 589-607 of SEQ ID NO:1 (N11), 631-649 of SEQ ID NO:1 (N14), 645-663 of SEQ ID NO:1 (N15), 659-677 of SEQ ID NO:1 (N16), 673-691 of SEQ ID NO:1 (N17), 701-719 of SEQ ID NO:1 (N19), 715-733 of SEQ ID NO:1 (N20), 729-747 of SEQ ID NO:1 (N21), 743-761 of SEQ ID NO:1 (N22), 757-775 of SEQ ID NO:1 (N23), 771-789 of SEQ ID NO:1 (N24), 785-803 of SEQ ID NO:1 (N25), 799-817 of SEQ ID NO:1 (N26), 813-831 of SEQ ID NO:1 (N27), 827-845 of SEQ ID NO:1 (N28), 869-887 of SEQ ID NO:1 (C2), 883-901 of SEQ ID NO:1 (C3), 911-929 of SEQ ID NO:1 (C5), 925-943 of SEQ ID NO:1 (C6), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1065-1083 of SEQ ID NO:1 (C16), 1079-1097 of SEQ ID NO:1 (C17), 1107-1125 of SEQ ID NO:1 (C19), 1121-1139 of SEQ ID NO:1 (C20), 1135-1153 of SEQ ID NO:1 (C21), 1149-1167 of SEQ ID NO:1 (C22), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28), 1247-1265 of SEQ ID NO:1 (C29), 1261-1279 of SEQ ID NO:1 (C30), or 1275-1296 of SEQ ID NO:1 (C31), or an immunoreactive fragment thereof, with the proviso that the BoNT/A peptide is not SEQ ID NO:2. In an aspect of this embodiment, such a BoNT/A peptide is selected from one of the following amino acid sequences: 463-481 of SEQ ID NO:1 (N2), 519-537 of SEQ ID NO:l (N6), 533-551 of SEQ ID NO:1 (N7), 547-565 of SEQ ID NO:1 (N8), 561-579 of SEQ ID NO:1 (N9), 589-607 of SEQ ID NO:1 (N11), 659-677 of SEQ ID NO:1 (N16), 743-761 of SEQ ID NO:1 (N22), 785-803 of SEQ ID NO:1 (N25), 813-831 of SEQ ID NO:1 (N27), 827-845 of SEQ ID NO:1 (N28), 869-887 of SEQ ID NO:1 (C2), 883-901 of SEQ ID NO:1 (C3), 911-929 of SEQ ID NO:1 (C6), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1121-1139 of SEQ ID NO:1 (C20), 1135-1153 of SEQ ID NO:1 (C21), 1177-1195 of SEQ ID NO:1 (C24), 1261-1279 of SEQ ID NO:1 (C30) or 1275-1296 of SEQ ID NO:1 (C31), or an immunoreactive fragment thereof, with the proviso that the BoNT/A peptide is not SEQ ID NO:2. In another aspect of this embodiment, such a BoNT/A peptide is selected from one of the following amino acid sequences: 463-481 of SEQ ID NO:1 (N2), 533-551 of SEQ ID NO:1 (N7), 547-565 of SEQ ID NO:1 (N8), 561-579 of SEQ ID NO:1 (N9), 659-677 of SEQ ID NO:1 (N16), 743-761 of SEQ ID NO:1 (N22), 785-803 of SEQ ID NO:1 (N25), 813-831 of SEQ ID NO:1 (N27), 883-901 of SEQ ID NO:1 (C3), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1121-1139 of SEQ ID NO:1 (C20) or 1177-1195 of SEQ ID NO:1 (C24) or 1275-1296 of SEQ ID NO: 1 (C31), or an immunoreactive fragment thereof, with the proviso that the BoNT/A peptide is not SEQ ID NO:2. In another aspect of this embodiment, such a BoNT/A peptide is selected from one of the following amino acid sequences: 533-551 of SEQ ID NO: 1 (N7), 547-565 of SEQ ID NO: 1 (N8), 743-761 of SEQ ID NO: 1 (N22), 785-803 of SEQ ID NO: 1 (N25), 813-831 of SEQ ID NO: 1 (N27), 995-1013 of SEQ ID NO: 1 (C11); 1051-1069 of SEQ ID NO: 1 (C15), 1177-1195 of SEQ ID NO: 1 (C24), or 1275-1296 of SEQ ID NO: 1 (C31), or an immunoreactive fragment thereof, with the proviso that the BoNT/A peptide is not SEQ ID NO:2.

In yet another aspect of this embodiment, a BoNT/A peptide useful in a method disclosed in the present specification for preparing an anti-BoNT/A antibody composition has a length of at most 60 amino acids and consisting of at least 5 contiguous amino acids selected from one of the following BoNT/A amino acid sequences: 463-481 of SEQ ID NO:1 (N2), 505-523 of SEQ ID NO:1 (N5), 519-537 of SEQ ID NO:1 (N6), 533-551 of SEQ ID NO:1 (N7), 603-621 of SEQ ID NO:1 (N12), 645-663 of SEQ ID NO:1 (N15), 659-677 of SEQ ID NO:1 (N16), 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1079-1097 of SEQ ID NO:1 (C17), 1107-1125 of SEQ ID NO:1 (C19), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28) or 1275-1296 of SEQ ID NO:1 (C31), or an immunoreactive fragment thereof, with the proviso that the BoNT/A peptide is not SEQ ID NO:2. In yet another aspect of this embodiment, such a BoNT/A peptide is selected from one of the following amino acid sequences: 533-551 of SEQ ID NO:1 (N7), 659-677 of SEQ ID NO:1 (N16), 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1107-1125 of SEQ ID NO:l (C19), 1163-1181 of SEQ ID NO:1 (C23), 1191-1209 of SEQ ID NO:1 (C25), 1233-1251 of SEQ ID NO:1 (C28) or 1275-1296 of SEQ ID NO:1 (C31), or an immunoreactive fragment thereof, with the proviso that the BoNT/A peptide is not SEQ ID NO:2. In yet another aspect of this embodiment, such a BoNT/A peptide is selected from one of the following amino acid sequences: 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1079-1097 of SEQ ID NO:1 (C17), 1107-1125 of SEQ ID NO:1 (C19), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28) or 1275-1296 of SEQ ID NO:1 (C31), or an immunoreactive fragment thereof, with the proviso that the BoNT/A peptide is not SEQ ID NO:2. In yet another aspect of this embodiment, such a BoNT/A peptide is selected from one of the following amino acid sequences: 729-747 of SEQ ID NO:1 (N21) 799-817 of SEQ ID NO:1 (N26), 1065-1083 of SEQ ID NO:1 (C16), 1163-1181 of SEQ ID NO:1 (C23) or 1275-1296 of SEQ ID NO:1 (C31), or an immunoreactive fragment thereof, with the proviso that the BoNT/A peptide is not SEQ ID NO:2.

In is envisioned that a BoNT/A peptide useful in a method disclosed in the present specification for preparing an anti-BoNT/A antibody composition can have any of a variety of lengths from at least 5 amino acids to at most 60 amino acids. Therefore, aspects of this embodiment may include a BoNT/A peptide with at least, e.g., five amino acids, six amino acids, seven amino acids, eight amino acids, nine amino acids, ten amino acids, 11 amino acids, 12 amino acids, 13 amino acids, 14 amino acids, 15 amino acids, 16 amino acids, 17 amino acids, 18 amino acids, 19 amino acids, 20 amino acids, 25 amino acids, 30 amino acids, 35 amino acids, 40 amino acids, 45 amino acids, 50 amino acids, 55 amino acids or 60 amino acids. Other aspects of this embodiment may include a BoNT/A peptide with at least, e.g., five amino acids of SEQ ID NO:1, six amino acids of SEQ ID NO:1, seven amino acids of SEQ ID NO:1, eight amino acids of SEQ ID NO:1, nine amino acids of SEQ ID NO:1, ten amino acids of SEQ ID NO:1, 11 amino acids of SEQ ID NO:1, 12 amino acids of SEQ ID NO:1, 13 amino acids of SEQ ID NO:1, 14 amino acids of SEQ ID NO:1, 15 amino acids of SEQ ID NO:1, 16 amino acids of SEQ ID NO:1, 17 amino acids of SEQ ID NO:1, 18 amino acids of SEQ ID NO:1, 19 amino acids of SEQ ID NO:1, 20 amino acids of SEQ ID NO:1, 25 amino acids of SEQ ID NO:1, 30 amino acids of SEQ ID NO:1, 35 amino acids of SEQ ID NO:1, 40 amino acids of SEQ ID NO:1, 45 amino acids of SEQ ID NO:1, 50 amino acids of SEQ ID NO:1, 55 amino acids or 60 amino acids of SEQ ID NO:1. In further embodiments, such a BoNT/A peptide of the invention may include a BoNT/A peptide with at least, e.g., five amino acids, six amino acids, seven amino acids, eight amino acids, nine amino acids, ten amino acids, 11 amino acids, 12 amino acids, 13 amino acids, 14 amino acids, 15 amino acids, 16 amino acids, 17 amino acids, 18 amino acids, 19 amino acids, 20 amino acids, 25 amino acids, 30 amino acids, 35 amino acids, 40 amino acids, 45 amino acids, 50 amino acids, 55 amino acids or 60 amino acids and consist of at least 5 contiguous amino acids selected from one of the following BoNT/A amino acid sequences: 449-467 of SEQ ID NO:1(N1), 463-481 of SEQ ID NO:1 (N2), 491-509 of SEQ ID NO:1 (N4), 505-523 of SEQ ID NO:1 (N5), 519-537 of SEQ ID NO:1 (N6), 533-551 of SEQ ID NO:1 (N7), 547-565 of SEQ ID NO:1 (N8), 561-579 of SEQ ID NO:1 (N9), 575-593 of SEQ ID NO:1 (N10), 589-607 of SEQ ID NO:1 (N11), 631-649 of SEQ ID NO:1 (N14), 645-663 of SEQ ID NO:1 (N15), 659-677 of SEQ ID NO:1 (N16), 673-691 of SEQ ID NO:1 (N17), 701-719 of SEQ ID NO:1 (N19), 715-733 of SEQ ID NO:1 (N20), 729-747 of SEQ ID NO:1 (N21), 743-761 of SEQ ID NO:1 (N22), 757-775 of SEQ ID NO:1 (N23), 771-789 of SEQ ID NO:1 (N24), 785-803 of SEQ ID NO:1 (N25), 799-817 of SEQ ID NO:1 (N26), 813-831 of SEQ ID NO:1 (N27), 827-845 of SEQ ID NO:1 (N28), 869-887 of SEQ ID NO:1 (C2), 883-901 of SEQ ID NO:1 (C3), 911-929 of SEQ ID NO:1 (C5), 925-943 of SEQ ID NO:1 (C6), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1065-1083 of SEQ ID NO:1 (C16), 1079-1097 of SEQ ID NO:1 (C17), 1107-1125 of SEQ ID NO:1 (C19), 1121-1139 of SEQ ID NO:1 (C20), 1135-1153 of SEQ ID NO:1 (C21), 1149-1167 of SEQ ID NO:1 (C22), 1163-1181 of SEQ ID NO:1 (C23), 1177-1195 of SEQ ID NO:1 (C24),1191-1209 of SEQ ID NO:1 (C25), 1233-1251 of SEQ ID NO:1 (C28), 1247-1265 of SEQ ID NO:1 (C29), 1261-1279 of SEQ ID NO:1 (C30) or 1275-1296 of SEQ ID NO:1 (C31), or a conservative variant, a non-conservative variant, or immunoreactive fragment thereof, with the proviso that the BoNT/A peptide is not SEQ ID NO:2.

In another embodiment of the present invention, a BoNT/A peptide useful in a method disclosed in the present specification for preparing an anti-BoNT/A antibody composition can comprise one BoNT/A peptide disclosed in the present specification. In another embodiment of the present invention, a BoNT/A peptide useful in a method disclosed in the present specification for preventing or reducing BoNT toxicity can comprise a plurality of BoNT/A peptides disclosed in the present specification. Thus, aspects of this embodiment can include one or more BoNT/A peptides, two or more BoNT/A peptides, three or more BoNT/A peptides, four or more BoNT/A peptides, five or more BoNT/A peptides, six or more BoNT/A peptides, seven or more BoNT/A peptides, eight or more BoNT/A peptides, nine or more BoNT/A peptides, ten or more BoNT/A peptides, 15 or more BoNT/A peptides, 20 or more BoNT/A peptides, 25 or more BoNT/A peptides or 30 or more BoNT/A peptides. In other aspects of this embodiment can include one or more BoNT/A conservative variants, two or more BoNT/A conservative variants, three or more BoNT/A conservative variants, four or more BoNT/A conservative variants, five or more BoNT/A conservative variants, six or more BoNT/A conservative variants, seven or more BoNT/A conservative variants, eight or more BoNT/A conservative variants, nine or more BoNT/A conservative variants, ten or more BoNT/A conservative variants, 15 or more BoNT/A conservative variants, 20 or more BoNT/A conservative variants, 25 or more BoNT/A conservative variants or 30 or more BoNT/A conservative variants. In further aspects of this embodiment can include one or more BoNT/A non-conservative variants, two or more BoNT/A non-conservative variants, three or more BoNT/A non-conservative variants, four or more BoNT/A non-conservative variants, five or more BoNT/A non-conservative variants, six or more BoNT/A non-conservative variants, seven or more BoNT/A non-conservative variants, eight or more BoNT/A non-conservative variants, nine or more BoNT/A non-conservative variants, ten or more BoNT/A non-conservative variants, 15 or more BoNT/A non-conservative variants, 20 or more BoNT/A non-conservative variants, 25 or more BoNT/A non-conservative variants or 30 or more BoNT/A non-conservative variants. In still other aspects of this embodiment can include one or more BoNT/A immunoreactive fragments, two or more BoNT/A immunoreactive fragments, three or more BoNT/A immunoreactive fragments, four or more BoNT/A immunoreactive fragments, five or more BoNT/A immunoreactive fragments, six or more BoNT/A immunoreactive fragments, seven or more BoNT/A immunoreactive fragments, eight or more BoNT/A immunoreactive fragments, nine or more BoNT/A peptides, ten or more BoNT/A immunoreactive fragments, 15 or more BoNT/A immunoreactive fragments, 20 or more BoNT/A immunoreactive fragments, 25 or more BoNT/A immunoreactive fragments or 30 or more BoNT/A immunoreactive fragments. BoNT/A peptides disclosed in the present specification useful for preparing an anti-BoNT/A antibody composition can be selected, for example, depending on immunological factors, such as potency of the peptide in inducing an immune response, and technical factors, such as chemical synthesis yields. It is also understood that the two or more BoNT/A peptides can be provided separately or as part of a compound molecule such as a chimeric peptide or heterologous protein.

In an aspect of this embodiment, a method of preparing an anti-BoNT/A antibody composition uses two or more immunoreactive BoNT/A peptides selected from the following amino acid sequences: 533-551 of SEQ ID NO:1 (N7), 547-565 of SEQ ID NO:1 (N8), 743-761 of SEQ ID NO:1 (N22), 785-803 of SEQ ID NO:1 (N25), 813-831 of SEQ ID NO:1 (N27), 995-1013 of SEQ ID NO:1 (C11); 1051-1069 of SEQ ID NO:l (C15), 1177-1195 of SEQ ID NO:1 (C24), and 1275-1296 of SEQ ID NO:1 (C31), or a conservative variant, a non-conservative variant or an immunoreactive fragment thereof, with the proviso that the BoNT/A peptide is not SEQ ID NO:2. In another aspect of this embodiment, one of the selected amino acid sequence is 533-551 of SEQ ID NO:1 (N8) or a conservative variant, a non-conservative variant or an immunoreactive fragment thereof, with the proviso that the BoNT/A peptide is not SEQ ID NO:2. In yet another aspect of this embodiment, the following two amino acid sequences are selected: 533-551 of SEQ ID NO:1 (N8) and 981-999 of SEQ ID NO:1 (C10), or a conservative variant, a non-conservative variant or an immunoreactive fragment thereof, with the proviso that the BoNT/A peptide is not SEQ ID NO:2. In yet another aspect of this embodiment, the following three amino acid sequences are selected: 533-551 of SEQ ID NO:1 (N8), 981-999 of SEQ ID NO:1 (C10) and 1051-1069 of SEQ ID NO:1 (C15), or a conservative variant, a non-conservative variant or an immunoreactive fragment thereof, with the proviso that the BoNT/A peptide is not SEQ ID NO:2. In a further aspect of this embodiment, one of the amino acid sequences selected is 785-803 of SEQ ID NO:1 (N25) or a conservative variant, a non-conservative variant or an immunoreactive fragment thereof, with the proviso that the BoNT/A peptide is not SEQ ID NO:2. In a still further aspect of this embodiment, the following two amino acid sequences are selected: 785-803 of SEQ ID NO:1 (N25) and 981-999 of SEQ ID NO:1 (C10), or a conservative variant, a non-conservative variant or an immunoreactive fragment thereof, with the proviso that the BoNT/A peptide is not SEQ ID NO:2. In a still further aspect of this embodiment, the following three amino acid sequences are selected: 785-803 of SEQ ID NO:1 (N25), 981-999 of SEQ ID NO:1 (C10) and 1051-1069 of SEQ ID NO:1 (C15), or a conservative variant, a non-conservative variant or an immunoreactive fragment thereof, with the proviso that the BoNT/A peptide is not SEQ ID NO:2. In an additional aspect of this embodiment, one of the amino acid sequences selected is 813-831 of SEQ ID NO:1 (N27) or a conservative variant, a non-conservative variant or an immunoreactive fragment thereof, with the proviso that the BoNT/A peptide is not SEQ ID NO:2. In another additional aspect of this embodiment, the following two amino acid sequences are selected: 813-831 of SEQ ID NO:1 (N27) and 981-999 of SEQ ID NO:1 (C10), or a conservative variant, a non-conservative variant or an immunoreactive fragment thereof, with the proviso that the BoNT/A peptide is not SEQ ID NO:2. In another additional aspect of this embodiment, the following three amino acid sequences are selected: 813-831 of SEQ ID NO:1 (N27), 981-999 of SEQ ID NO:1 (C10) and 1051-1069 of SEQ ID NO:1 (C15), or a conservative variant, a non-conservative variant or an immunoreactive fragment thereof, with the proviso that the BoNT/A peptide is not SEQ ID NO:2.

In an aspect of this embodiment, a method of preparing an anti-BoNT/A antibody composition uses two or more immunoreactive BoNT/A peptides selected from the following amino acid sequences: 659-677 of SEQ ID NO:1 (N16), 729-747 of SEQ ID NO:1 (N21), 799-817 of SEQ ID NO:1 (N26), 1065-1083 of SEQ ID NO:1 (C16), 1163-1181 of SEQ ID NO:1 (C23), and 1275-1296 of SEQ ID NO:1 (C31), or a conservative variant, a non-conservative variant or an immunoreactive fragment thereof, with the proviso that the BoNT/A peptide is not SEQ ID NO:2. In another aspect of this embodiment, one of the amino acid sequences selected is 1065-1083 of SEQ ID NO:1 (C16) or a conservative variant, a non-conservative variant or an immunoreactive fragment thereof, with the proviso that the BoNT/A peptide is not SEQ ID NO:2. In yet another aspect of this embodiment, the following two amino acid sequences are selected: 1065-1083 of SEQ ID NO:l (C16) and 1163-1181 of SEQ ID NO:1 (C23), or a conservative variant, a non-conservative variant or an immunoreactive fragment thereof, with the proviso that the BoNT/A peptide is not SEQ ID NO:2. In yet another aspect of this embodiment, the following three amino acid sequences are selected: 1065-1083 of SEQ ID NO:1 (C16), 1163-1181 of SEQ ID NO:1 (C23) and 1275-1296 of SEQ ID NO:1 (C31), or a conservative variant, a non-conservative variant or an immunoreactive fragment thereof, with the proviso that the BoNT/A peptide is not SEQ ID NO:2. In a further aspect of this embodiment, one of the amino acid sequences selected is 799-817 of SEQ ID NO:1 (N26) or a conservative variant, a non-conservative variant or an immunoreactive fragment thereof, with the proviso that the BoNT/A peptide is not SEQ ID NO:2. In a still further aspect of this embodiment, the following two amino acid sequences are selected: 799-817 of SEQ ID NO:1 (N26) and 1065-1083 of SEQ ID NO:1 (C16), or a conservative variant, a non-conservative variant or an immunoreactive fragment thereof, with the proviso that the BoNT/A peptide is not SEQ ID NO:2. In a still further aspect of this embodiment, the following three amino acid sequences are selected: 799-817 of SEQ ID NO:1 (N26), 1065-1083 of SEQ ID NO:1 (C16) and 1163-1181 of SEQ ID NO:1 (C23), or a conservative variant, a non-conservative variant or an immunoreactive fragment thereof, with the proviso that the BoNT/A peptide is not SEQ ID NO:2. In an additional aspect of this embodiment, one of the amino acid sequences selected is 729-747 of SEQ ID NO:1 (N21) or a conservative variant, a non-conservative variant or an immunoreactive fragment thereof, with the proviso that the BoNT/A peptide is not SEQ ID NO:2. In another additional aspect of this embodiment, the following two amino acid sequences are selected: 729-747 of SEQ ID NO:1 (N21) and 1065-1083 of SEQ ID NO:1 (C16), or a conservative variant, a non-conservative variant or an immunoreactive fragment thereof, with the proviso that the BoNT/A peptide is not SEQ ID NO:2. In another additional aspect of this embodiment, the following three amino acid sequences are selected: 729-747 of SEQ ID NO:1 (N21), 1065-1083 of SEQ ID NO:1 (C16) and 1163-1181 of SEQ ID NO:1 (C23), or a conservative variant, a non-conservative variant or an immunoreactive fragment thereof, with the proviso that the BoNT/A peptide is not SEQ ID NO:2.

It is also envisioned that any and all combinations of BoNT/A peptides disclosed in the specification can be useful for preparing an anti-BoNT/A antibody composition, including, e.g., BoNT/A peptides of SEQ ID NO:1, BoNT/A conservative variants, BoNT/A non-conservative variants and BoNT/A immunoreactive fragments. Thus, aspects of this embodiment include one or more BoNT/A peptides comprising one or more BoNT/A peptides of SEQ ID NO: 1 and one or more BoNT/A conservative variants; one or more BoNT/A peptides of SEQ ID NO: 1 and one or more BoNT/A non-conservative variants; one or more BoNT/A peptides of SEQ ID NO: 1 and one or more BoNT/A immunoreactive fragments; one or more BoNT/A conservative variants and one or more BoNT/A non-conservative variants; one or more BoNT/A conservative variants and one or more BoNT/A immunoreactive fragments; one or more BoNT/A non-conservative variants and one or more BoNT/A immunoreactive fragments; one or more BoNT/A peptides of SEQ ID NO: 1, one or more BoNT/A conservative variants and one or more BoNT/A non-conservative variants; one or more BoNT/A peptides of SEQ ID NO: 1, one or more BoNT/A conservative variants and one or more BoNT/A immunoreactive fragments; one or more BoNT/A peptides of SEQ ID NO: 1, one or more BoNT/A non-conservative variants and one or more BoNT/A immunoreactive fragments; one or more BoNT/A conservative variants, one or more BoNT/A non-conservative variants and one or more BoNT/A immunoreactive fragments; or one or more BoNT/A peptides of SEQ ID NO: 1, one or more BoNT/A conservative variants, one or more BoNT/A non-conservative variants and one or more BoNT/A immunoreactive fragments.

The term “antibody”, as used herein, includes polyclonal and monoclonal antibodies, as well as antigenic compound-binding fragments of such antibodies including, without limitation, Fab, F(ab′).sub.2, Fd, Fv fragments, and single chain derivatives of the same. “Antibody” also includes cell-associated antibodies, such as Ig receptors, for example. In addition, the term “antibody” includes naturally occurring antibodies, as well as non-naturally occurring antibodies, including, for example, chimeric, bifunctional, and humanized antibodies, and related synthetic isoforms. As used herein, an “epitope” means the site on an antigen that is recognized and bound by a particular antibody or T-cell receptor. The minimal size of a protein epitope, as defined herein, is about five amino acids, and a protein epitope typically comprises at least eight amino acids. It is to be noted, however, that an epitope might comprise a portion of an antigen other than the amino acid sequence, e.g., a carbohydrate moiety or a lipid moiety. Furthermore, an epitope may be discontinuous, i.e., it comprises amino acid residues that are not adjacent in the polypeptide but are brought together into an epitope by way of the secondary, tertiary, or quaternary structure of the protein. As used herein, the term “selectively binds” means the discriminatory binding of the antibody to the indicated target peptide or polypeptide such that the antibody does not substantially cross react with unrelated peptides or polypeptides. Specific reactivity can include binding properties such as binding specificity, binding affinity and binding avidity. For example, an antibody can bind a target peptide or polypeptide with a binding affinity (Kd) of about 10⁻⁴ M or more, 10⁻⁶ M or more, 10⁻⁷ M or more, 10⁻⁸ M or more, 10⁻⁹ M or more, or 10⁻¹⁰ M or more. Several methods for detecting or measuring antibody binding are known in the art and disclosed herein.

Monoclonal antibodies refer to a homogeneous population of antibody molecules that contain only one species of antibody capable of binding a particular antigen. By definition, a monoclonal antibody binds to a single epitope. Methods of producing a monoclonal antibody are well known, see, e.g., Harlow & Lane, supra, 1998a; and Harlow & Lane, supra, 1998b. As a non-limiting example, a hybridoma that produces a BoNT/A monoclonal antibody can be identified by screening hybridoma supernatants for the presence of antibodies that bind to a BoNT/A peptide of the invention, see, e.g., Harlow & Lane, supra, 1998a; and Harlow & Lane, supra, 1998b. For example, hybridoma supernatants can be screened using BoNT/A-positive sera in a radioimmunoassay or enzyme-linked immunosorbent assay. Polyclonal antibodies refer to a heterogeneous population of antibody molecules that contain two or more species of antibody capable of binding to a particular antigen. Methods of producing a polyclonal antibody are well known, see, e.g., Harlow & Lane, supra, 1 998a; and Harlow & Lane, supra, 1998b. As a non-limiting example, serum from an animal immunized with a BoNT/A peptide of the invention can be screened in a radioimmunoassay or enzyme-linked immunosorbent assay to identify a polyclonal BoNT/A antibody.

A variety of well known methods can be used for collecting from an animal a sample containing an antibody or antibody-producing cell. Such methods are described, see, e.g., Harlow & Lane, supra, 1998a; and Harlow & Lane, supra, 1998b. Similarly, a variety of well known methods can be used for processing a sample to isolate an anti-BoNT/A antibody. A procedure for collecting an processing a sample can be selected based on the type of antibody to be isolated. As an example, when isolating polyclonal antibodies, an appropriate sample can be a blood sample containing antibodies, whereas when isolating monoclonal antibodies, an appropriate sample can be an antibody-producing cell such as a spleen cell. Exemplary well known procedures for isolating both monoclonal and polyclonal antibodies are known in the art art as described above. In still further embodiments, any of the monoclonal antibodies disclosed above are of the IgG subtype.

X. Method of Treating Botulinum Toxicity in an Individual

Botulinum toxicity refers to intoxication resulting from exposure to botulinum toxin. Botulism clinical syndromes include food borne botulism, which can result from ingestion of preformed botulinum toxin in contaminated foods; wound botulism, which can result from the production of botulinum toxin in vivo after growth of C. botulinum in an infected wound; GI colonization syndromes, which can result from the production of botulinum toxin in vivo due after growth of C. botulinum in the intestinal tract of a colonized individual; iatrogenic botulism, which can result from injection of botulinum toxin into a tissue of an individual; and inhalation botulism, which can occur accidentally in humans, for example, in a veterinary setting when working with infected animals, and as a result of biological warfare. The signs and symptoms of botulinum intoxication are well known to those skilled in the art. Experiments performed in vivo and in vitro indicate that antibodies can enter cholinergic nerves and neutralize internalized BoNT, see, e.g., Lance L. Simpson, The Study of Clostridial and Related Toxins. The Search for Unique Mechanisms and Common Denominators, 84(2) J. PHYSIOL. (PARIS) 143-51 (1990). As such, anti-BoNT antibodies can act, for example, extracellularly by interfering with the binding of BoNT to the cell surface and intracellularly by interfering with BoNT enzymatic activity. A BoNT/A antibody prepared according to a method of the invention can bind to a botulinum toxin and neutralize its effects. Thus, the present invention provides a method of treating botulinum toxicity in a human or other mammal by administering to the human or other mammal a pharmaceutical composition comprising an anti-BoNT/A antibody composition disclosed in the present specification.

In an embodiment of the present invention, an anti-BoNT/A antibody composition useful in a method disclosed in the present specification for preventing or reducing BoNT toxicity is prepared from a BoNT/A peptide having a length of at most 60 amino acids and consisting of at least 5 contiguous amino acids selected from one of the following BoNT/A amino acid sequences: 449-467 of SEQ ID NO:1(N1), 463-481 of SEQ ID NO:1 (N2), 491-509 of SEQ ID NO:1 (N4), 505-523 of SEQ ID NO:1 (N5), 519-537 of SEQ ID NO:1 (N6), 533-551 of SEQ ID NO:1 (N7), 547-565 of SEQ ID NO:1 (N8), 561-579 of SEQ ID NO:1 (N9), 575-593 of SEQ ID NO:1 (N10), 589-607 of SEQ ID NO:1 (N11), 631-649 of SEQ ID NO:1 (N14), 645-663 of SEQ ID NO:1 (N15), 659-677 of SEQ ID NO:1 (N16), 673-691 of SEQ ID NO:1 (N17), 701-719 of SEQ ID NO:1 (N19), 715-733 of SEQ ID NO:1 (N20), 729-747 of SEQ ID NO:1 (N21), 743-761 of SEQ ID NO:1 (N22), 757-775 of SEQ ID NO:1 (N23), 771-789 of SEQ ID NO:1 (N24), 785-803 of SEQ ID NO:1 (N25), 799-817 of SEQ ID NO:1 (N26), 813-831 of SEQ ID NO:1 (N27), 827-845 of SEQ ID NO:1 (N28), 869-887 of SEQ ID NO:1 (C2), 883-901 of SEQ ID NO:1 (C3), 911-929 of SEQ ID NO:1 (C5), 925-943 of SEQ ID NO:1 (C6), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15),1065-1083 of SEQ ID NO:1 (C16), 1079-1097 of SEQ ID NO:1 (C17), 1107-1125 of SEQ ID NO:1 (C19), 1121-1139 of SEQ ID NO:l (C20), 1135-1153 of SEQ ID NO:1 (C21), 1149-1167 of SEQ ID NO:1 (C22), 1163-1181 of SEQ ID NO:1 (C23), 1177-1195 of SEQ ID NO:1 (C24),1191-1209 of SEQ ID NO:1 (C25), 1233-1251 of SEQ ID NO:1 (C28), 1247-1265 of SEQ ID NO:1 (C29), 1261-1279 of SEQ ID NO:1 (C30), or 1275-1296 of SEQ ID NO:1 (C31), or a conservative variant or immunoreactive fragment thereof, with the proviso that the BoNT/A peptide is not SEQ ID NO:2.

In another embodiment of the present invention, an anti-BoNT/A antibody composition useful in a method disclosed in the present specification for preventing or reducing BoNT toxicity is prepared from a BoNT/A peptide having a length of at most 60 amino acids and consisting of at least 5 contiguous amino acids selected from one of the following BoNT/A amino acid sequences: 449-467 of SEQ ID NO:1(N1), 463-481 of SEQ ID NO:1 (N2), 491-509 of SEQ ID NO:1 (N4), 505-523 of SEQ ID NO:1 (N5), 519-537 of SEQ ID NO:1 (N6), 533-551 of SEQ ID NO:1 (N7), 547-565 of SEQ ID NO:1 (N8), 561-579 of SEQ ID NO:1 (N9), 575-593 of SEQ ID NO:1 (N10), 589-607 of SEQ ID NO:1 (N11), 631-649 of SEQ ID NO:1 (N14), 645-663 of SEQ ID NO:1 (N15), 659-677 of SEQ ID NO:1 (N16), 673-691 of SEQ ID NO:1 (N17), 701-719 of SEQ ID NO:1 (N19), 715-733 of SEQ ID NO:1 (N20), 729-747 of SEQ ID NO:1 (N21), 743-761 of SEQ ID NO:1 (N22), 757-775 of SEQ ID NO:1 (N23), 771-789 of SEQ ID NO:1 (N24), 785-803 of SEQ ID NO:1 (N25), 799-817 of SEQ ID NO:1 (N26), 813-831 of SEQ ID NO:1 (N27), 827-845 of SEQ ID NO:1 (N28), 869-887 of SEQ ID NO:1 (C2), 883-901 of SEQ ID NO:1 (C3), 911-929 of SEQ ID NO:1 (C5), 925-943 of SEQ ID NO:1 (C6), 939-957 of SEQ ID NO:1 (C7), 967-985 of SEQ ID NO:1 (C9), 981-999 of SEQ ID NO:1 (C10), 995-1013 of SEQ ID NO:1 (C1), 1051-1069 of SEQ ID NO:1 (C15), 1065-1083 of SEQ ID NO:1 (C16), 1079-1097 of SEQ ID NO:1 (C17), 1107-1125 of SEQ ID NO:1 (C19), 1121-1139 of SEQ ID NO:1 (C20), 1135-1153 of SEQ ID NO:1 (C21), 1149-1167 of SEQ ID NO:1 (C22), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28),1247-1265 of SEQ ID NO:1 (C29),1261-1279 of SEQ ID NO:1 (C30), or 1275-1296 of SEQ ID NO:1 (C31). In an aspect of this embodiment, such a BoNT/A peptide is selected from one of the following amino acid sequences: 463-481 of SEQ ID NO:1 (N2), 519-537 of SEQ ID NO:1 (N6), 533-551 of SEQ ID NO:1 (N7), 547-565 of SEQ ID NO:1 (N8), 561-579 of SEQ ID NO:1 (N9), 589-607 of SEQ ID NO:1 (N11), 659-677 of SEQ ID NO:1 (N16), 743-761 of SEQ ID NO:1 (N22), 785-803 of SEQ ID NO:1 (N25), 813-831 of SEQ ID NO:1 (N27),827-845 of SEQ ID NO:1 (N28),869-887 of SEQ ID NO:1 (C2),883-901 of SEQ ID NO:1 (C3), 911-929 of SEQ ID NO:1 (C6), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1121-1139 of SEQ ID NO:1 (C20), 1135-1153 of SEQ ID NO:1 (C21), 1177-1195 of SEQ ID NO:1 (C24),1261-1279 of SEQ ID NO:1 (C30) or 1275-1296 of SEQ ID NO:1 (C31). In another aspect of this embodiment, such a BoNT/A peptide is selected from one of the following amino acid sequences: 463-481 of SEQ ID NO:l (N2), 533-551 of SEQ ID NO:1 (N7), 547-565 of SEQ ID NO:1 (N8), 561-579 of SEQ ID NO:1 (N9), 659-677 of SEQ ID NO:1 (N16), 743-761 of SEQ ID NO:1 (N22), 785-803 of SEQ ID NO:1 (N25), 813-831 of SEQ ID NO:1 (N27), 883-901 of SEQ ID NO:1 (C3), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1121-1139 of SEQ ID NO:1 (C20) or 1177-1195 of SEQ ID NO:1 (C24) or 1275-1296 of SEQ ID NO: 1 (C31). In another aspect of this embodiment, such a BoNT/A peptide is selected from one of the following amino acid sequences: 533-551 of SEQ ID NO: 1 (N7), 547-565 of SEQ ID NO: 1 (N8), 743-761 of SEQ ID NO: 1 (N22), 785-803 of SEQ ID NO: 1 (N25), 813-831 of SEQ ID NO: 1 (N27), 995-1013 of SEQ ID NO: 1 (C11); 1051-1069 of SEQ ID NO: 1 (C15), 1177-1195 of SEQ ID NO: 1 (C24), or 1275-1296 of SEQ ID NO: 1 (C31).

In yet another aspect of this embodiment, an anti-BoNT/A antibody composition useful in a method disclosed in the present specification for preventing or reducing BoNT toxicity is prepared from a BoNT/A peptide having a length of at most 60 amino acids and consisting of at least 5 contiguous amino acids selected from one of the following BoNT/A amino acid sequences: 463-481 of SEQ ID NO:1 (N2), 505-523 of SEQ ID NO:1 (N5), 519-537 of SEQ ID NO:1 (N6), 533-551 of SEQ ID NO:1 (N7), 603-621 of SEQ ID NO:1 (N12), 645-663 of SEQ ID NO:1 (N15), 659-677 of SEQ ID NO:1 (N16), 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1079-1097 of SEQ ID NO:1 (C17), 1107-1125 of SEQ ID NO:1 (C19), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28) or 1275-1296 of SEQ ID NO:1 (C31). In yet another aspect of this embodiment, such a BoNT/A peptide is selected from one of the following amino acid sequences: 533-551 of SEQ ID NO:1 (N7), 659-677 of SEQ ID NO:1 (N16), 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1107-1125 of SEQ ID NO:1 (C19), 1163-1181 of SEQ ID NO:1 (C23), 1191-1209 of SEQ ID NO:1 (C25), 1233-1251 of SEQ ID NO:1 (C28) or 1275-1296 of SEQ ID NO:1 (C31). In yet another aspect of this embodiment, such a BoNT/A peptide is selected from one of the following amino acid sequences: 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1079-1097 of SEQ ID NO:1 (C17), 1107-1125 of SEQ ID NO:1 (C19), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28) or 1275-1296 of SEQ ID NO:1 (C31). In yet another aspect of this embodiment, such a BoNT/A peptide is selected from one of the following amino acid sequences: 729-747 of SEQ ID NO:1 (N21) 799-817 of SEQ ID NO:1 (N26), 1065-1083 of SEQ ID NO:1 (C16), 1163-1181 of SEQ ID NO:1 (C23) or 1275-1296 of SEQ ID NO:1 (C31).

In yet another embodiment of the present invention, an anti-BoNT/A antibody composition useful in a method disclosed in the present specification for preventing or reducing BoNT toxicity is prepared from a BoNT/A peptide selected from one of the following BoNT/A amino acid sequences: 449-467 of SEQ ID NO:1(N1), 463-481 of SEQ ID NO:1 (N2), 491-509 of SEQ ID NO:1 (N4), 505-523 of SEQ ID NO:1 (N5), 519-537 of SEQ ID NO:1 (N6), 533-551 of SEQ ID NO:1 (N7), 547-565 of SEQ ID NO:1 (N8), 561-579 of SEQ ID NO:1 (N9), 575-593 of SEQ ID NO:1 (N10), 589-607 of SEQ ID NO:1 (N11), 631-649 of SEQ ID NO:1 (N14), 645-663 of SEQ ID NO:1 (N15), 659-677 of SEQ ID NO:1 (N16), 673-691 of SEQ ID NO:1 (N17), 701-719 of SEQ ID NO:1 (N19), 715-733 of SEQ ID NO:1 (N20), 729-747 of SEQ ID NO:1 (N21), 743-761 of SEQ ID NO:1 (N22), 757-775 of SEQ ID NO:1 (N23), 771-789 of SEQ ID NO:1 (N24), 785-803 of SEQ ID NO:1 (N25), 799-817 of SEQ ID NO:1 (N26), 813-831 of SEQ ID NO:1 (N27), 827-845 of SEQ ID NO:1 (N28), 869-887 of SEQ ID NO:1 (C2), 883-901 of SEQ ID NO:1 (C3), 911-929 of SEQ ID NO:1 (C5), 925-943 of SEQ ID NO:1 (C6), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1065-1083 of SEQ ID NO:1 (C16), 1079-1097 of SEQ ID NO:1 (C17), 1107-1125 of SEQ ID NO:1 (C19), 1121-1139 of SEQ ID NO:1 (C20), 1135-1153 of SEQ ID NO:1 (C21), 1149-1167 of SEQ ID NO:l (C22), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28), 1247-1265 of SEQ ID NO:1 (C29), 1261-1279 of SEQ ID NO:1 (C30), or 1275-1296 of SEQ ID NO:1 (C31). In an aspect of this embodiment, such a BoNT/A peptide is selected from one of the following amino acid sequences: 463-481 of SEQ ID NO:1 (N2), 519-537 of SEQ ID NO:1 (N6), 533-551 of SEQ ID NO:1 (N7), 547-565 of SEQ ID NO:1 (N8), 561-579 of SEQ ID NO:1 (N9), 589-607 of SEQ ID NO:1 (N11), 659-677 of SEQ ID NO:1 (N16), 743-761 of SEQ ID NO:1 (N22), 785-803 of SEQ ID NO:1 (N25), 813-831 of SEQ ID NO:1 (N27), 827-845 of SEQ ID NO:1 (N28), 869-887 of SEQ ID NO:1 (C2), 883-901 of SEQ ID NO:1 (C3), 911-929 of SEQ ID NO:1 (C6), 939-957 of SEQ ID NO:1 (C7), 967-985 of SEQ ID NO:1 (C9), 981-999 of SEQ ID NO:1 (C10), 995-1013 of SEQ ID NO:l (C11), 1051-1069 of SEQ ID NO:1 (C15), 1121-1139 of SEQ ID NO:1 (C20), 1135-1153 of SEQ ID NO:1 (C21), 1177-1195 of SEQ ID NO:1 (C24), 1261-1279 of SEQ ID NO:1 (C30) or 1275-1296 of SEQ ID NO:1 (C31). In another aspect of this embodiment, such a BoNT/A peptide is selected from one of the following amino acid sequences: 463-481 of SEQ ID NO:1 (N2), 533-551 of SEQ ID NO:1 (N7), 547-565 of SEQ ID NO:1 (N8), 561-579 of SEQ ID NO:1 (N9), 659-677 of SEQ ID NO:1 (N16), 743-761 of SEQ ID NO:1 (N22), 785-803 of SEQ ID NO:1 (N25), 813-831 of SEQ ID NO:1 (N27), 883-901 of SEQ ID NO:1 (C3), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1121-1139 of SEQ ID NO:1 (C20) or 1177-1195 of SEQ ID NO:1 (C24) or 1275-1296 of SEQ ID NO: 1 (C31). In another aspect of this embodiment, such a BoNT/A peptide is selected from one of the following amino acid sequences: 533-551 of SEQ ID NO: 1 (N7), 547-565 of SEQ ID NO: 1 (N8), 743-761 of SEQ ID NO: 1 (N22), 785-803 of SEQ ID NO: 1 (N25), 813-831 of SEQ ID NO: 1 (N27), 995-1013 of SEQ ID NO: 1 (Cl 1); 1051-1069 of SEQ ID NO: 1 (C15), 1177-1195 of SEQ ID NO: 1 (C24), or 1275-1296 of SEQ ID NO: 1 (C31).

In yet another aspect of this embodiment, an anti-BoNT/A antibody composition useful in a method disclosed in the present specification for preventing or reducing BoNT toxicity is prepared from a BoNT/A peptide selected from one of the following BoNT/A amino acid sequences: 463-481 of SEQ ID NO:1 (N2), 505-523 of SEQ ID NO:1 (N5), 519-537 of SEQ ID NO:1 (N6), 533-551 of SEQ ID NO:1 (N7), 603-621 of SEQ ID NO:1 (N12), 645-663 of SEQ ID NO:1 (N15), 659-677 of SEQ ID NO:1 (N16), 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1079-1097 of SEQ ID NO:1 (C17), 1107-1125 of SEQ ID NO:1 (C19), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28) or 1275-1296 of SEQ ID NO:1 (C31). In yet another aspect of this embodiment, such a BoNT/A peptide is selected from one of the following amino acid sequences: 533-551 of SEQ ID NO:1 (N7), 659-677 of SEQ ID NO:1 (N16), 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1107-1125 of SEQ ID NO:1 (C19), 1163-1181 of SEQ ID NO:1 (C23), 1191-1209 of SEQ ID NO:1 (C25), 1233-1251 of SEQ ID NO:1 (C28) or 1275-1296 of SEQ ID NO:1 (C31). In yet another aspect of this embodiment, such a BoNT/A peptide is selected from one of the following amino acid sequences: 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1079-1097 of SEQ ID NO:1 (C17), 1107-1125 of SEQ ID NO:1 (C19), 1163-1181 of SEQ ID NO:1 (C23), 1177-1195 of SEQ ID NO:1 (C24),1191-1209 of SEQ ID NO:1 (C25), 1233-1251 of SEQ ID NO:1 (C28) or 1275-1296 of SEQ ID NO:1 (C31). In yet another aspect of this embodiment, such a BoNT/A peptide is selected from one of the following amino acid sequences: 729-747 of SEQ ID NO:1 (N21) 799-817 of SEQ ID NO:1 (N26), 1065-1083 of SEQ ID NO:1 (C16), 1163-1181 of SEQ ID NO:1 (C23) or 1275-1296 of SEQ ID NO:1 (C31).

In yet another embodiment of the present invention, an anti-BoNT/A antibody composition useful in a method disclosed in the present specification for preventing or reducing BoNT toxicity is prepared from a BoNT/A peptide having a length of at most 60 amino acids and consisting of at least 5 contiguous amino acids selected from one of the following BoNT/A amino acid sequences: 449-467 of SEQ ID NO:1(N1), 463-481 of SEQ ID NO:1 (N2), 491-509 of SEQ ID NO:1 (N4), 505-523 of SEQ ID NO:1 (N5), 519-537 of SEQ ID NO:1 (N6), 533-551 of SEQ ID NO:1 (N7), 547-565 of SEQ ID NO:1 (N8), 561-579 of SEQ ID NO:1 (N9), 575-593 of SEQ ID NO:1 (N10), 589-607 of SEQ ID NO:1 (N11), 631-649 of SEQ ID NO:1 (N14), 645-663 of SEQ ID NO:1 (N15), 659-677 of SEQ ID NO:1 (N16), 673-691 of SEQ ID NO:1 (N17), 701-719 of SEQ ID NO:1 (N19), 715-733 of SEQ ID NO:1 (N20), 729-747 of SEQ ID NO:1 (N21), 743-761 of SEQ ID NO:1 (N22), 757-775 of SEQ ID NO:1 (N23), 771-789 of SEQ ID NO:1 (N24), 785-803 of SEQ ID NO:1 (N25), 799-817 of SEQ ID NO:1 (N26), 813-831 of SEQ ID NO:1 (N27), 827-845 of SEQ ID NO:1 (N28), 869-887 of SEQ ID NO:1 (C2), 883-901 of SEQ ID NO:1 (C3), 911-929 of SEQ ID NO:1 (C5), 925-943 of SEQ ID NO:1 (C6), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1065-1083 of SEQ ID NO:1 (C16), 1079-1097 of SEQ ID NO:1 (C17), 1107-1125 of SEQ ID NO:1 (C19), 1121-1139 of SEQ ID NO:1 (C20), 1135-1153 of SEQ ID NO:1 (C21), 1149-1167 of SEQ ID NO:1 (C22), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28), 1247-1265 of SEQ ID NO:1 (C29), 1261-1279 of SEQ ID NO:1 (C30), or 1275-1296 of SEQ ID NO:1 (C31), or a conservative variant thereof. In an aspect of this embodiment, such a BoNT/A peptide is selected from one of the following amino acid sequences: 463-481 of SEQ ID NO:1 (N2), 519-537 of SEQ ID NO:1 (N6), 533-551 of SEQ ID NO:1 (N7), 547-565 of SEQ ID NO:1 (N8), 561-579 of SEQ ID NO:1 (N9), 589-607 of SEQ ID NO:1 (N11), 659-677 of SEQ ID NO:1 (N16), 743-761 of SEQ ID NO:1 (N22), 785-803 of SEQ ID NO:1 (N25), 813-831 of SEQ ID NO:1 (N27), 827-845 of SEQ ID NO:1 (N28), 869-887 of SEQ ID NO:1 (C2), 883-901 of SEQ ID NO:1 (C3), 911-929 of SEQ ID NO:l (C6), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1121-1139 of SEQ ID NO:1 (C20), 1135-1153 of SEQ ID NO:1 (C21), 1177-1195 of SEQ ID NO:1 (C24), 1261-1279 of SEQ ID NO:1 (C30) or 1275-1296 of SEQ ID NO:l (C31), or a conservative variant thereof. In another aspect of this embodiment, such a BoNT/A peptide is selected from one of the following amino acid sequences: 463-481 of SEQ ID NO:1 (N2), 533-551 of SEQ ID NO:1 (N7), 547-565 of SEQ ID NO:1 (N8), 561-579 of SEQ ID NO:1 (N9), 659-677 of SEQ ID NO:1 (N16), 743-761 of SEQ ID NO:1 (N22), 785-803 of SEQ ID NO:1 (N25), 813-831 of SEQ ID NO:1 (N27), 883-901 of SEQ ID NO:1 (C3), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1121-1139 of SEQ ID NO:1 (C20) or 1177-1195 of SEQ ID NO:1 (C24) or 1275-1296 of SEQ ID NO: 1 (C31), or a conservative variant thereof. In another aspect of this embodiment, such a BoNT/A peptide is selected from one of the following amino acid sequences: 533-551 of SEQ ID NO: 1 (N7), 547-565 of SEQ ID NO: 1 (N8), 743-761 of SEQ ID NO: 1 (N22), 785-803 of SEQ ID NO: 1 (N25), 813-831 of SEQ ID NO: 1 (N27), 995-1013 of SEQ ID NO: 1 (C11); 1051-1069 of SEQ ID NO: 1 (C15), 1177-1195 of SEQ ID NO: 1 (C24), or 1275-1296 of SEQ ID NO: 1 (C31), or a conservative variant thereof.

In yet another aspect of this embodiment, an anti-BoNT/A antibody composition useful in a method disclosed in the present specification for preventing or reducing BoNT toxicity is prepared from a BoNT/A peptide having a length of at most 60 amino acids and consisting of at least 5 contiguous amino acids selected from one of the following BoNT/A amino acid sequences: 463-481 of SEQ ID NO:1 (N2), 505-523 of SEQ ID NO:1 (N5), 519-537 of SEQ ID NO:1 (N6), 533-551 of SEQ ID NO:1 (N7), 603-621 of SEQ ID NO:1 (N12), 645-663 of SEQ ID NO:1 (N15), 659-677 of SEQ ID NO:1 (N16), 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1079-1097 of SEQ ID NO:1 (C17), 1107-1125 of SEQ ID NO:1 (C19), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28) or 1275-1296 of SEQ ID NO:1 (C31), or a conservative variant thereof. In yet another aspect of this embodiment, such a BoNT/A peptide is selected from one of the following amino acid sequences: 533-551 of SEQ ID NO:1 (N7), 659-677 of SEQ ID NO:1 (N16), 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1107-1125 of SEQ ID NO:1 (C19), 1163-1181 of SEQ ID NO:1 (C23), 1191-1209 of SEQ ID NO:1 (C25), 1233-1251 of SEQ ID NO:1 (C28) or 1275-1296 of SEQ ID NO:1 (C31), or a conservative variant thereof. In yet another aspect of this embodiment, such a BoNT/A peptide is selected from one of the following amino acid sequences: 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1079-1097 of SEQ ID NO:1 (C17), 1107-1125 of SEQ ID NO:1 (C19), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28) or 1275-1296 of SEQ ID NO:1 (C31), or a conservative variant thereof. In yet another aspect of this embodiment, such a BoNT/A peptide is selected from one of the following amino acid sequences: 729-747 of SEQ ID NO:1 (N21) 799-817 of SEQ ID NO:1 (N26), 1065-1083 of SEQ ID NO:1 (C16), 1163-1181 of SEQ ID NO:1 (C23) or 1275-1296 of SEQ ID NO:1 (C31), or a conservative variant thereof.

In yet another embodiment of the present invention, an anti-BoNT/A antibody composition useful in a method disclosed in the present specification for preventing or reducing BoNT toxicity is prepared from a BoNT/A peptide having a length of at most 60 amino acids and consisting of at least 5 contiguous amino acids selected from one of the following BoNT/A amino acid sequences: 449-467 of SEQ ID NO:1(N1), 463-481 of SEQ ID NO:1 (N2), 491-509 of SEQ ID NO:1 (N4), 505-523 of SEQ ID NO:1 (N5), 519-537 of SEQ ID NO:1 (N6), 533-551 of SEQ ID NO:1 (N7), 547-565 of SEQ ID NO:1 (N8), 561-579 of SEQ ID NO:1 (N9), 575-593 of SEQ ID NO:1 (N10), 589-607 of SEQ ID NO:1 (N11), 631-649 of SEQ ID NO:1 (N14), 645-663 of SEQ ID NO:1 (N15), 659-677 of SEQ ID NO:1 (N16), 673-691 of SEQ ID NO:1 (N17), 701-719 of SEQ ID NO:1 (N19), 715-733 of SEQ ID NO:1 (N20), 729-747 of SEQ ID NO:1 (N21), 743-761 of SEQ ID NO:1 (N22), 757-775 of SEQ ID NO:1 (N23), 771-789 of SEQ ID NO:1 (N24), 785-803 of SEQ ID NO:1 (N25), 799-817 of SEQ ID NO:1 (N26), 813-831 of SEQ ID NO:1 (N27), 827-845 of SEQ ID NO:1 (N28), 869-887 of SEQ ID NO:1 (C2), 883-901 of SEQ ID NO:1 (C3), 911-929 of SEQ ID NO:1 (C5), 925-943 of SEQ ID NO:1 (C6), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1065-1083 of SEQ ID NO:1 (C16), 1079-1097 of SEQ ID NO:1 (C17), 1107-1125 of SEQ ID NO:1 (C19), 1121-1139 of SEQ ID NO:1 (C20), 1135-1153 of SEQ ID NO:1 (C21), 1149-1167 of SEQ ID NO:1 (C22), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28), 1247-1265 of SEQ ID NO:1 (C29), 1261-1279 of SEQ ID NO:1 (C30), or 1275-1296 of SEQ ID NO:l (C31), or a non-conservative variant thereof. In an aspect of this embodiment, such a BoNT/A peptide is selected from one of the following amino acid sequences: 463-481 of SEQ ID NO:1 (N2), 519-537 of SEQ ID NO:1 (N6), 533-551 of SEQ ID NO:1 (N7), 547-565 of SEQ ID NO:1 (N8), 561-579 of SEQ ID NO:1 (N9), 589-607 of SEQ ID NO:1 (N11), 659-677 of SEQ ID NO:1 (N16), 743-761 of SEQ ID NO:l (N22), 785-803 of SEQ ID NO:1 (N25), 813-831 of SEQ ID NO:1 (N27), 827-845 of SEQ ID NO:1 (N28), 869-887 of SEQ ID NO:1 (C2), 883-901 of SEQ ID NO:1 (C3), 911-929 of SEQ ID NO:1 (C6), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1121-1139 of SEQ ID NO:1 (C20), 1135-1153 of SEQ ID NO:1 (C21), 1177-1195 of SEQ ID NO:1 (C24), 1261-1279 of SEQ ID NO:1 (C30) or 1275-1296 of SEQ ID NO:l (C31), or a non-conservative variant thereof. In another aspect of this embodiment, such a BoNT/A peptide is selected from one of the following amino acid sequences: 463-481 of SEQ ID NO:1 (N2), 533-551 of SEQ ID NO:1 (N7), 547-565 of SEQ ID NO:1 (N8), 561-579 of SEQ ID NO:1 (N9), 659-677 of SEQ ID NO:1 (N16), 743-761 of SEQ ID NO:1 (N22), 785-803 of SEQ ID NO:1 (N25), 813-831 of SEQ ID NO:1 (N27), 883-901 of SEQ ID NO:1 (C3), 939-957 of SEQ ID NO:1 (C7), 967-985 of SEQ ID NO:l (C9), 981-999 of SEQ ID NO:1 (C10), 995-1013 of SEQ ID NO:1 (C11), 1051-1069 of SEQ ID NO:1 (C15), 1121-1139 of SEQ ID NO:1 (C20) or 1177-1195 of SEQ ID NO:1 (C24) or 1275-1296 of SEQ ID NO: 1 (C31), or a non-conservative variant thereof. In another aspect of this embodiment, such a BoNT/A peptide is selected from one of the following amino acid sequences: 533-551 of SEQ ID NO: 1 (N7), 547-565 of SEQ ID NO: 1 (N8), 743-761 of SEQ ID NO: 1 (N22), 785-803 of SEQ ID NO: 1 (N25), 813-831 of SEQ ID NO: 1 (N27), 995-1013 of SEQ ID NO: 1 (C11); 1051-1069 of SEQ ID NO: 1 (C15), 1177-1195 of SEQ ID NO: 1 (C24), or 1275-1296 of SEQ ID NO: 1 (C31), or a non-conservative variant thereof.

In yet another aspect of this embodiment, an anti-BoNT/A antibody composition useful in a method disclosed in the present specification for preventing or reducing BoNT toxicity is prepared from a BoNT/A peptide having a length of at most 60 amino acids and consisting of at least 5 contiguous amino acids selected from one of the following BoNT/A amino acid sequences: 463-481 of SEQ ID NO:1 (N2), 505-523 of SEQ ID NO:1 (N5), 519-537 of SEQ ID NO:1 (N6), 533-551 of SEQ ID NO:1 (N7), 603-621 of SEQ ID NO:1 (N12), 645-663 of SEQ ID NO:1 (N15), 659-677 of SEQ ID NO:1 (N16), 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1079-1097 of SEQ ID NO:1 (C17), 1107-1125 of SEQ ID NO:1 (C19), 1163-1181 of SEQ ID NO:1 (C23), 1177-1195 of SEQ ID NO:l (C24), 1191-1209 of SEQ ID NO:1 (C25), 1233-1251 of SEQ ID NO:1 (C28) or 1275-1296 of SEQ ID NO:1 (C31), or a non- conservative variant thereof. In yet another aspect of this embodiment, such a BoNT/A peptide is selected from one of the following amino acid sequences: 533-551 of SEQ ID NO:1 (N7), 659-677 of SEQ ID NO:1 (N16), 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1107-1125 of SEQ ID NO:1 (C19), 1163-1181 of SEQ ID NO:1 (C23), 1191-1209 of SEQ ID NO:1 (C25), 1233-1251 of SEQ ID NO:1 (C28) or 1275-1296 of SEQ ID NO:1 (C31), or a non-conservative variant thereof. In yet another aspect of this embodiment, such a BoNT/A peptide is selected from one of the following amino acid sequences: 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1079-1097 of SEQ ID NO:l (C17), 1107-1125 of SEQ ID NO:1 (C19), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28) or 1275-1296 of SEQ ID NO:1 (C31), or a non-conservative variant thereof. In yet another aspect of this embodiment, such a BoNT/A peptide is selected from one of the following amino acid sequences: 729-747 of SEQ ID NO:1 (N21) 799-817 of SEQ ID NO:1 (N26), 1065-1083 of SEQ ID NO:1 (C16), 1163-1181 of SEQ ID NO:1 (C23) or 1275-1296 of SEQ ID NO:1 (C31), or a non-conservative variant thereof.

In yet another embodiment of the present invention, an anti-BoNT/A antibody composition useful in a method disclosed in the present specification for preventing or reducing BoNT toxicity is prepared from a BoNT/A peptide having a length of at most 60 amino acids and consisting of at least 5 contiguous amino acids selected from one of the following BoNT/A amino acid sequences: 449-467 of SEQ ID NO:1(N1), 463-481 of SEQ ID NO:1 (N2), 491-509 of SEQ ID NO:1 (N4), 505-523 of SEQ ID NO:1 (N5), 519-537 of SEQ ID NO:1 (N6), 533-551 of SEQ ID NO:1 (N7), 547-565 of SEQ ID NO:1 (N8), 561-579 of SEQ ID NO:1 (N9), 575-593 of SEQ ID NO:1 (N10), 589-607 of SEQ ID NO:1 (N11), 631-649 of SEQ ID NO:1 (N14), 645-663 of SEQ ID NO:1 (N15), 659-677 of SEQ ID NO:1 (N16), 673-691 of SEQ ID NO:1 (N17), 701-719 of SEQ ID NO:1 (N19), 715-733 of SEQ ID NO:1 (N20), 729-747 of SEQ ID NO:1 (N21), 743-761 of SEQ ID NO:1 (N22), 757-775 of SEQ ID NO:1 (N23), 771-789 of SEQ ID NO:1 (N24), 785-803 of SEQ ID NO:1 (N25), 799-817 of SEQ ID NO:1 (N26), 813-831 of SEQ ID NO:1 (N27), 827-845 of SEQ ID NO:1 (N28), 869-887 of SEQ ID NO:1 (C2), 883-901 of SEQ ID NO:1 (C3), 911-929 of SEQ ID NO:1 (C5), 925-943 of SEQ ID NO:1 (C6), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1065-1083 of SEQ ID NO:1 (C16), 1079-1097 of SEQ ID NO:1 (C17), 1107-1125 of SEQ ID NO:1 (C19), 1121-1139 of SEQ ID NO:1 (C20), 1135-1153 of SEQ ID NO:1 (C21), 1149-1167 of SEQ ID NO:1 (C22), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28), 1247-1265 of SEQ ID NO:1 (C29), 1261-1279 of SEQ ID NO:1 (C30), or 1275-1296 of SEQ ID NO:1 (C31), or an immunoreactive fragment thereof, with the proviso that the BoNT/A peptide is not SEQ ID NO:2. In an aspect of this embodiment, such a BoNT/A peptide is selected from one of the following amino acid sequences: 463-481 of SEQ ID NO:1 (N2), 519-537 of SEQ ID NO:1 (N6), 533-551 of SEQ ID NO:1 (N7), 547-565 of SEQ ID NO:1 (N8), 561-579 of SEQ ID NO:1 (N9), 589-607 of SEQ ID NO:1 (N11), 659-677 of SEQ ID NO:1 (N16), 743-761 of SEQ ID NO:1 (N22), 785-803 of SEQ ID NO:1 (N25), 813-831 of SEQ ID NO:1 (N27), 827-845 of SEQ ID NO:1 (N28), 869-887 of SEQ ID NO:1 (C2), 883-901 of SEQ ID NO:1 (C3), 911-929 of SEQ ID NO:1 (C6), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1121-1139 of SEQ ID NO:1 (C20), 1135-1153 of SEQ ID NO:1 (C21), 1177-1195 of SEQ ID NO:1 (C24), 1261-1279 of SEQ ID NO:1 (C30) or 1275-1296 of SEQ ID NO:1 (C31), or an immunoreactive fragment thereof, with the proviso that the BoNT/A peptide is not SEQ ID NO:2. In another aspect of this embodiment, such a BoNT/A peptide is selected from one of the following amino acid sequences: 463-481 of SEQ ID NO:1 (N2), 533-551 of SEQ ID NO:1 (N7), 547-565 of SEQ ID NO:1 (N8), 561-579 of SEQ ID NO:1 (N9), 659-677 of SEQ ID NO:1 (N16), 743-761 of SEQ ID NO:1 (N22), 785-803 of SEQ ID NO:1 (N25), 813-831 of SEQ ID NO:1 (N27), 883-901 of SEQ ID NO:1 (C3), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1121-1139 of SEQ ID NO:1 (C20) or 1177-1195 of SEQ ID NO:1 (C24) or 1275-1296 of SEQ ID NO: 1 (C31), or an immunoreactive fragment thereof, with the proviso that the BoNT/A peptide is not SEQ ID NO:2. In another aspect of this embodiment, such a BoNT/A peptide is selected from one of the following amino acid sequences: 533-551 of SEQ ID NO: 1 (N7), 547-565 of SEQ ID NO: 1 (N8), 743-761 of SEQ ID NO: 1 (N22), 785-803 of SEQ ID NO: 1 (N25), 813-831 of SEQ ID NO: 1 (N27), 995-1013 of SEQ ID NO: 1 (C11); 1051-1069 of SEQ ID NO: 1 (Cl 5), 1177-1195 of SEQ ID NO: 1 (C24), or 1275-1296 of SEQ ID NO: 1 (C31), or an immunoreactive fragment thereof, with the proviso that the BoNT/A peptide is not SEQ ID NO:2.

In yet another aspect of this embodiment, an anti-BoNT/A antibody composition useful in a method disclosed in the present specification for preventing or reducing BoNT toxicity is prepared from a BoNT/A peptide having a length of at most 60 amino acids and consisting of at least 5 contiguous amino acids selected from one of the following BoNT/A amino acid sequences: 463-481 of SEQ ID NO:1 (N2), 505-523 of SEQ ID NO:1 (N5), 519-537 of SEQ ID NO:1 (N6), 533-551 of SEQ ID NO:1 (N7), 603-621 of SEQ ID NO:1 (N12), 645-663 of SEQ ID NO:1 (N15), 659-677 of SEQ ID NO:1 (N16), 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1079-1097 of SEQ ID NO:1 (C17), 1107-1125 of SEQ ID NO:1 (C19), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28) or 1275-1296 of SEQ ID NO:1 (C31), or an immunoreactive fragment thereof, with the proviso that the BoNT/A peptide is not SEQ ID NO:2. In yet another aspect of this embodiment, such a BoNT/A peptide is selected from one of the following amino acid sequences: 533-551 of SEQ ID NO:1 (N7), 659-677 of SEQ ID NO:1 (N16), 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1107-1125 of SEQ ID NO:1 (C19), 1163-1181 of SEQ ID NO:1 (C23), 1191-1209 of SEQ ID NO:1 (C25), 1233-1251 of SEQ ID NO:1 (C28) or 1275-1296 of SEQ ID NO:1 (C31), or an immunoreactive fragment thereof, with the proviso that the BoNT/A peptide is not SEQ ID NO:2. In yet another aspect of this embodiment, such a BoNT/A peptide is selected from one of the following amino acid sequences: 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1079-1097 of SEQ ID NO:1 (C17), 1107-1125 of SEQ ID NO:1 (C19), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28) or 1275-1296 of SEQ ID NO:1 (C31), or an immunoreactive fragment thereof, with the proviso that the BoNT/A peptide is not SEQ ID NO:2. In yet another aspect of this embodiment, such a BoNT/A peptide is selected from one of the following amino acid sequences: 729-747 of SEQ ID NO:1 (N21) 799-817 of SEQ ID NO:1 (N26), 1065-1083 of SEQ ID NO:1 (C16), 1163-1181 of SEQ ID NO:1 (C23) or 1275-1296 of SEQ ID NO:1 (C31), or an immunoreactive fragment thereof, with the proviso that the BoNT/A peptide is not SEQ ID NO:2.

In is envisioned that a BoNT/A peptide useful in a method disclosed in the present specification for preparing an antibody composition useful for preventing or reducing BoNT toxicity can have any of a variety of lengths from at least 5 amino acids to at most 60 amino acids. Therefore, aspects of this embodiment may include a BoNT/A peptide with at least, e.g., five amino acids, six amino acids, seven amino acids, eight amino acids, nine amino acids, ten amino acids, 11 amino acids, 12 amino acids, 13 amino acids, 14 amino acids, 15 amino acids, 16 amino acids, 17 amino acids, 18 amino acids, 19 amino acids, 20 amino acids, 25 amino acids, 30 amino acids, 35 amino acids, 40 amino acids, 45 amino acids, 50 amino acids, 55 amino acids or 60 amino acids. Other aspects of this embodiment may include a BoNT/A peptide with at least, e.g., five amino acids of SEQ ID NO:1, six amino acids of SEQ ID NO:1, seven amino acids of SEQ ID NO:1, eight amino acids of SEQ ID NO:1, nine amino acids of SEQ ID NO:1, ten amino acids of SEQ ID NO:1, 11 amino acids of SEQ ID NO:1, 12 amino acids of SEQ ID NO:1, 13 amino acids of SEQ ID NO:1, 14 amino acids of SEQ ID NO:1, 15 amino acids of SEQ ID NO:1, 16 amino acids of SEQ ID NO:1, 17 amino acids of SEQ ID NO:1, 18 amino acids of SEQ ID NO:1, 19 amino acids of SEQ ID NO:1, 20 amino acids of SEQ ID NO:1, 25 amino acids of SEQ ID NO:1, 30 amino acids of SEQ ID NO:1, 35 amino acids of SEQ ID NO:1, 40 amino acids of SEQ ID NO:1, 45 amino acids of SEQ ID NO:1, 50 amino acids of SEQ ID NO:1, 55 amino acids or 60 amino acids of SEQ ID NO:1. In further embodiments, such a BoNT/A peptide of the invention may include a BoNT/A peptide with at least, e.g., five amino acids, six amino acids, seven amino acids, eight amino acids, nine amino acids, ten amino acids, 11 amino acids, 12 amino acids, 13 amino acids, 14 amino acids, 15 amino acids, 16 amino acids, 17 amino acids, 18 amino acids, 19 amino acids, 20 amino acids, 25 amino acids, 30 amino acids, 35 amino acids, 40 amino acids, 45 amino acids, 50 amino acids, 55 amino acids or 60 amino acids and consist of at least 5 contiguous amino acids selected from one of the following BoNT/A amino acid sequences: 449-467 of SEQ ID NO:1 (N1), 463-481 of SEQ ID NO:1 (N2), 491-509 of SEQ ID NO:1 (N4), 505-523 of SEQ ID NO:1 (N5), 519-537 of SEQ ID NO:1 (N6), 533-551 of SEQ ID NO:1 (N7), 547-565 of SEQ ID NO:1 (N8), 561-579 of SEQ ID NO:1 (N9), 575-593 of SEQ ID NO:1 (N10), 589-607 of SEQ ID NO:1 (N11), 631-649 of SEQ ID NO:1 (N14), 645-663 of SEQ ID NO:1 (N15), 659-677 of SEQ ID NO:1 (N16), 673-691 of SEQ ID NO:1 (N17), 701-719 of SEQ ID NO:1 (N19), 715-733 of SEQ ID NO:1 (N20), 729-747 of SEQ ID NO:1 (N21), 743-761 of SEQ ID NO:1 (N22), 757-775 of SEQ ID NO:1 (N23), 771-789 of SEQ ID NO:1 (N24), 785-803 of SEQ ID NO:1 (N25), 799-817 of SEQ ID NO:1 (N26), 813-831 of SEQ ID NO:1 (N27), 827-845 of SEQ ID NO:1 (N28), 869-887 of SEQ ID NO:1 (C2), 883-901 of SEQ ID NO:1 (C3), 911-929 of SEQ ID NO:1 (C5), 925-943 of SEQ ID NO:1 (C6), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1065-1083 of SEQ ID NO:1 (C16), 1079-1097 of SEQ ID NO:1 (C17), 1107-1125 of SEQ ID NO:1 (C19), 1121-1139 of SEQ ID NO:1 (C20), 1135-1153 of SEQ ID NO:1 (C21), 1149-1167 of SEQ ID NO:1 (C22), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28), 1247-1265 of SEQ ID NO:1 (C29), 1261-1279 of SEQ ID NO:1 (C30) or 1275-1296 of SEQ ID NO:1 (C31), or a conservative variant, a non-conservative variant, or immunoreactive fragment thereof, with the proviso that the BoNT/A peptide is not SEQ ID NO:2.

In another embodiment of the present invention, a BoNT/A peptide useful in a method disclosed in the present specification for preparing an antibody composition useful for preventing or reducing BoNT toxicity can comprise one BoNT/A peptide disclosed in the present specification. In another embodiment of the present invention, a BoNT/A peptide useful in a method disclosed in the present specification for preparing an antibody composition useful for preventing or reducing BoNT toxicity can comprise a plurality of BoNT/A peptides disclosed in the present specification. Thus, aspects of this embodiment can include one or more BoNT/A peptides, two or more BoNT/A peptides, three or more BoNT/A peptides, four or more BoNT/A peptides, five or more BoNT/A peptides, six or more BoNT/A peptides, seven or more BoNT/A peptides, eight or more BoNT/A peptides, nine or more BoNT/A peptides, ten or more BoNT/A peptides, 15 or more BoNT/A peptides, 20 or more BoNT/A peptides, 25 or more BoNT/A peptides or 30 or more BoNT/A peptides. In other aspects of this embodiment can include one or more BoNT/A conservative variants, two or more BoNT/A conservative variants, three or more BoNT/A conservative variants, four or more BoNT/A conservative variants, five or more BoNT/A conservative variants, six or more BoNT/A conservative variants, seven or more BoNT/A conservative variants, eight or more BoNT/A conservative variants, nine or more BoNT/A conservative variants, ten or more BoNT/A conservative variants, 15 or more BoNT/A conservative variants, 20 or more BoNT/A conservative variants, 25 or more BoNT/A conservative variants or 30 or more BoNT/A conservative variants. In further aspects of this embodiment can include one or more BoNT/A non-conservative variants, two or more BoNT/A non-conservative variants, three or more BoNT/A non-conservative variants, four or more BoNT/A non-conservative variants, five or more BoNT/A non-conservative variants, six or more BoNT/A non-conservative variants, seven or more BoNT/A non-conservative variants, eight or more BoNT/A non-conservative variants, nine or more BoNT/A non-conservative variants, ten or more BoNT/A non-conservative variants, 15 or more BoNT/A non-conservative variants, 20 or more BoNT/A non-conservative variants, 25 or more BoNT/A non-conservative variants or 30 or more BoNT/A non-conservative variants. In still other aspects of this embodiment can include one or more BoNT/A immunoreactive fragments, two or more BoNT/A immunoreactive fragments, three or more BoNT/A immunoreactive fragments, four or more BoNT/A immunoreactive fragments, five or more BoNT/A immunoreactive fragments, six or more BoNT/A immunoreactive fragments, seven or more BoNT/A immunoreactive fragments, eight or more BoNT/A immunoreactive fragments, nine or more BoNT/A peptides, ten or more BoNT/A immunoreactive fragments, 15 or more BoNT/A immunoreactive fragments, 20 or more BoNT/A immunoreactive fragments, 25 or more BoNT/A immunoreactive fragments or 30 or more BoNT/A immunoreactive fragments. BoNT/A peptides disclosed in the present specification for preparing an antibody composition useful for preventing or reducing BoNT toxicity can be selected, for example, depending on immunological factors, such as potency of the peptide in inducing an immune response, and technical factors, such as chemical synthesis yields. It is also understood that the two or more BoNT/A peptides can be provided separately or as part of a compound molecule such as a chimeric peptide or heterologous protein.

In an aspect of this embodiment, a method of preventing or reducing BONT toxicity using an antibody composition can be prepared using two or more immunoreactive BoNT/A peptides selected from the following amino acid sequences: 533-551 of SEQ ID NO:1 (N7), 547-565 of SEQ ID NO:1 (N8), 743-761 of SEQ ID NO:1 (N22), 785-803 of SEQ ID NO:1 (N25), 813-831 of SEQ ID NO:1 (N27), 995-1013 of SEQ ID NO:1 (C11); 1051-1069 of SEQ ID NO:1 (C15), 1177-1195 of SEQ ID NO:1 (C24), and 1275-1296 of SEQ ID NO:1 (C31), or a conservative variant, a non-conservative variant or an immunoreactive fragment thereof, with the proviso that the BoNT/A peptide is not SEQ ID NO:2. In another aspect of this embodiment, one of the selected amino acid sequence is 533-551 of SEQ ID NO:1 (N8) or a conservative variant, a non-conservative variant or an immunoreactive fragment thereof, with the proviso that the BoNT/A peptide is not SEQ ID NO:2. In yet another aspect of this embodiment, the following two amino acid sequences are selected: 533-551 of SEQ ID NO:1 (N8) and 981-999 of SEQ ID NO:1 (C10), or a conservative variant, a non-conservative variant or an immunoreactive fragment thereof, with the proviso that the BoNT/A peptide is not SEQ ID NO:2. In yet another aspect of this embodiment, the following three amino acid sequences are selected: 533-551 of SEQ ID NO:1 (N8), 981-999 of SEQ ID NO:1 (C10) and 1051-1069 of SEQ ID NO:1 (C15), or a conservative variant, a non-conservative variant or an immunoreactive fragment thereof, with the proviso that the BoNT/A peptide is not SEQ ID NO:2. In a further aspect of this embodiment, one of the amino acid sequences selected is 785-803 of SEQ ID NO:1 (N25) or a conservative variant, a non-conservative variant or an immunoreactive fragment thereof, with the proviso that the BoNT/A peptide is not SEQ ID NO:2. In a still further aspect of this embodiment, the following two amino acid sequences are selected: 785-803 of SEQ ID NO:1 (N25) and 981-999 of SEQ ID NO:1 (C10), or a conservative variant, a non-conservative variant or an immunoreactive fragment thereof, with the proviso that the BoNT/A peptide is not SEQ ID NO:2. In a still further aspect of this embodiment, the following three amino acid sequences are selected: 785-803 of SEQ ID NO:1 (N25), 981-999 of SEQ ID NO:1 (C10) and 1051-1069 of SEQ ID NO:1 (C15), or a conservative variant, a non-conservative variant or an immunoreactive fragment thereof, with the proviso that the BoNT/A peptide is not SEQ ID NO:2. In an additional aspect of this embodiment, one of the amino acid sequences selected is 813-831 of SEQ ID NO:1 (N27) or a conservative variant, a non-conservative variant or an immunoreactive fragment thereof, with the proviso that the BoNT/A peptide is not SEQ ID NO:2. In another additional aspect of this embodiment, the following two amino acid sequences are selected: 813-831 of SEQ ID NO:1 (N27) and 981-999 of SEQ ID NO:1 (C10), or a conservative variant, a non-conservative variant or an immunoreactive fragment thereof, with the proviso that the BoNT/A peptide is not SEQ ID NO:2. In another additional aspect of this embodiment, the following three amino acid sequences are selected: 813-831 of SEQ ID NO:1 (N27), 981-999 of SEQ ID NO:1 (C10) and 1051-1069 of SEQ ID NO:1 (C15), or a conservative variant, a non-conservative variant or an immunoreactive fragment thereof, with the proviso that the BoNT/A peptide is not SEQ ID NO:2.

In an aspect of this embodiment, a method of preventing or reducing BoNT toxicity using an antibody composition can be prepared using two or more immunoreactive BoNT/A peptides selected from the following amino acid sequences: 659-677 of SEQ ID NO:1 (N16), 729-747 of SEQ ID NO:1 (N21), 799-817 of SEQ ID NO:1 (N26), 1065-1083 of SEQ ID NO:1 (C16), 1163-1181 of SEQ ID NO:1 (C23), and 1275-1296 of SEQ ID NO:1 (C31), or a conservative variant, a non-conservative variant or an immunoreactive fragment thereof, with the proviso that the BoNT/A peptide is not SEQ ID NO:2. In another aspect of this embodiment, one of the amino acid sequences selected is 1065-1083 of SEQ ID NO:1 (C16) or a conservative variant, a non-conservative variant or an immunoreactive fragment thereof, with the proviso that the BoNT/A peptide is not SEQ ID NO:2. In yet another aspect of this embodiment, the following two amino acid sequences are selected: 1065-1083 of SEQ ID NO:1 (C16) and 1163-1181 of SEQ ID NO:1 (C23), or a conservative variant, a non-conservative variant or an immunoreactive fragment thereof, with the proviso that the BoNT/A peptide is not SEQ ID NO:2. In yet another aspect of this embodiment, the following three amino acid sequences are selected: 1065-1083 of SEQ ID NO:1 (C16), 1163-1181 of SEQ ID NO:1 (C23) and 1275-1296 of SEQ ID NO:1 (C31), or a conservative variant, a non-conservative variant or an immunoreactive fragment thereof, with the proviso that the BoNT/A peptide is not SEQ ID NO:2. In a further aspect of this embodiment, one of the amino acid sequences selected is 799-817 of SEQ ID NO:1 (N26) or a conservative variant, a non-conservative variant or an immunoreactive fragment thereof, with the proviso that the BoNT/A peptide is not SEQ ID NO:2. In a still further aspect of this embodiment, the following two amino acid sequences are selected: 799-817 of SEQ ID NO:1 (N26) and 1065-1083 of SEQ ID NO:1 (C16), or a conservative variant, a non-conservative variant or an immunoreactive fragment thereof, with the proviso that the BoNT/A peptide is not SEQ ID NO:2. In a still further aspect of this embodiment, the following three amino acid sequences are selected: 799-817 of SEQ ID NO:1 (N26), 1065-1083 of SEQ ID NO:1 (C16) and 1163-1181 of SEQ ID NO:1 (C23), or a conservative variant, a non-conservative variant or an immunoreactive fragment thereof, with the proviso that the BoNT/A peptide is not SEQ ID NO:2. In an additional aspect of this embodiment, one of the amino acid sequences selected is 729-747 of SEQ ID NO:1 (N21) or a conservative variant, a non-conservative variant or an immunoreactive fragment thereof, with the proviso that the BoNT/A peptide is not SEQ ID NO:2. In another additional aspect of this embodiment, the following two amino acid sequences are selected: 729-747 of SEQ ID NO:1 (N21) and 1065-1083 of SEQ ID NO:1 (C16), or a conservative variant, a non-conservative variant or an immunoreactive fragment thereof, with the proviso that the BoNT/A peptide is not SEQ ID NO:2. In another additional aspect of this embodiment, the following three amino acid sequences are selected: 729-747 of SEQ ID NO:1 (N21), 1065-1083 of SEQ ID NO:1 (C16) and 1163-1181 of SEQ ID NO:1 (C23), or a conservative variant, a non-conservative variant or an immunoreactive fragment thereof, with the proviso that the BoNT/A peptide is not SEQ ID NO:2.

It is also envisioned that any and all combinations of BoNT/A peptides disclosed in the specification can be useful for preparing an antibody composition useful in a method of preventing or reducing BoNT toxicity, including, e.g., BoNT/A peptides of SEQ ID NO:1, BoNT/A conservative variants, BoNT/A non-conservative variants and BoNT/A immunoreactive fragments. Thus, aspects of this embodiment include one or more BoNT/A peptides comprising one or more BoNT/A peptides of SEQ ID NO: 1 and one or more BoNT/A conservative variants; one or more BoNT/A peptides of SEQ ID NO: 1 and one or more BoNT/A non-conservative variants; one or more BoNT/A peptides of SEQ ID NO: 1 and one or more BoNT/A immunoreactive fragments; one or more BoNT/A conservative variants and one or more BoNT/A non-conservative variants; one or more BoNT/A conservative variants and one or more BoNT/A immunoreactive fragments; one or more BoNT/A non-conservative variants and one or more BoNT/A immunoreactive fragments; one or more BoNT/A peptides of SEQ ID NO: 1, one or more BoNT/A conservative variants and one or more BoNT/A non-conservative variants; one or more BoNT/A peptides of SEQ ID NO: 1, one or more BoNT/A conservative variants and one or more BoNT/A immunoreactive fragments; one or more BoNT/A peptides of SEQ ID NO: 1, one or more BoNT/A non-conservative variants and one or more BoNT/A immunoreactive fragments; one or more BoNT/A conservative variants, one or more BoNT/A non-conservative variants and one or more BoNT/A immunoreactive fragments; or one or more BoNT/A peptides of SEQ ID NO: 1, one or more BoNT/A conservative variants, one or more BoNT/A non-conservative variants and one or more BoNT/A immunoreactive fragments.

One skilled in the art can determine if an anti-BoNT/A antibody composition induces an immune response, as methods for detecting immune responses are well known in the art. Non-limiting examples involve measuring the titer of anti-BoNT/A-selective antibodies in an animal primed with the antibody composition and boosted with the antigen, or determining the presence of antibodies in the blood of an immunized animal that are cross-reactive with the antigen by ELISA, Western blotting or other well-known methods. Cell-mediated immune responses can be determined, for example, by measuring cytotoxic T cell response to antigen using a variety of methods described hereinabove or well known in the art.

An anti-BoNT/A composition useful in a method of the invention can be administered by any of a variety of routes, as described below. Those skilled in the art can readily determine for a particular anti-BoNT/A composition, the appropriate antigen payload; route of immunization; volume of dose; and vaccination regimen useful in a particular animal, for example, humans.

As disclosed herein an anti-BoNT/A composition is administered to a human or other mammal to treat a condition characterized by BoNT/A toxicity. As used herein, the term “treating,” when used in reference to administering to a human or other mammal an effective amount of an anti-BoNT/A composition, means reducing a symptom of a condition characterized by BoNT/A toxicity, or delaying or preventing onset of a symptom of a condition characterized by BoNT/A toxicity in the human or other mammal. For example, the term “treating” can mean reducing a symptom of a condition characterized by BoNT/A toxicity by at least 30%, 40%, 60%, 70%, 80%, 90% or 100%. The effectiveness of an anti-BoNT/A composition in treating a condition characterized by BoNT/A toxicity can be determined by observing one or more clinical symptoms or physiological indicators associated with the condition. An improvement in a condition characterized by BoNT/A toxicity also can be indicated by a reduced need for a concurrent therapy. Those of skill in the art will know the appropriate symptoms or indicators associated with specific conditions and will know how to determine if a human or other mammal is a candidate for treatment with an anti-BoNT/A composition disclosed in the present specification. In particular, it is understood that those skilled in the art will be able to determine if a condition if characterized by BoNT/A toxicity, for example, by comparison of levels of BoNT/A toxicity from the normal control individuals.

The appropriate effective amount to be administered for a particular application of the methods can be determined by those skilled in the art, using the guidance provided herein. For example, an effective amount can be extrapolated from in vitro and in vivo assays as described herein above. One skilled in the art will recognize that the condition of the patient can be monitored throughout the course of therapy and that the effective amount of an anti-BoNT/A composition that is administered can be adjusted accordingly.

An anti-BoNT/A composition useful in the invention generally is administered in a pharmaceutical acceptable composition. As used herein, the term “pharmaceutically acceptable” refer to any molecular entity or composition that does not produce an adverse, allergic or other untoward or unwanted reaction when administered to a human or other mammal. As used herein, the term “pharmaceutically acceptable composition” refers to a therapeutically effective concentration of an active ingredient. A pharmaceutical composition may be administered to a patient alone, or in combination with other supplementary active ingredients, agents, drugs or hormones. The pharmaceutical compositions may be manufactured using any of a variety of processes, including, without limitation, conventional mixing, dissolving, granulating, dragee-making, levigating, emulsifying, encapsulating, entrapping, and lyophilizing. The pharmaceutical composition can take any of a variety of forms including, without limitation, a sterile solution, suspension, emulsion, lyophilizate, tablet, pill, pellet, capsule, powder, syrup, elixir or any other dosage form suitable for administration.

It is also envisioned that a pharmaceutical composition disclosed in the present specification can optionally include a pharmaceutically acceptable carriers that facilitate processing of an active ingredient into pharmaceutically acceptable compositions. As used herein, the term “pharmacologically acceptable carrier” refers to any carrier that has substantially no long term or permanent detrimental effect when administered and encompasses terms such as “pharmacologically acceptable vehicle, stabilizer, diluent, auxiliary or excipient.” Such a carrier generally is mixed with an active compound, or permitted to dilute or enclose the active compound and can be a solid, semi-solid, or liquid agent. It is understood that the active ingredients can be soluble or can be delivered as a suspension in the desired carrier or diluent. Any of a variety of pharmaceutically acceptable carriers can be used including, without limitation, aqueous media such as, e.g., distilled, deionized water, saline; solvents; dispersion media; coatings; antibacterial and antifungal agents; isotonic and absorption delaying agents; or any other inactive ingredient. Selection of a pharmacologically acceptable carrier can depend on the mode of administration. Except insofar as any pharmacologically acceptable carrier is incompatible with the active ingredient, its use in pharmaceutically acceptable compositions is contemplated. Non-limiting examples of specific uses of such pharmaceutical carriers can be found in PHARMACEUTICAL DOSAGE FORMS AND DRUG DELIVERY SYSTEMS (Howard C. Ansel et al., eds., Lippincott Williams & Wilkins Publishers, 7^{th} ed. 1999); REMINGTON: THE SCIENCE AND PRACTICE OF PHARMACY (Alfonso R. Gennaro ed., Lippincott, Williams & Wilkins, 20^{th} ed. 2000); GOODMAN & GILMAN'S THE PHARMACOLOGICAL BASIS OF THERAPEUTICS (Joel G. Hardman et al., eds., McGraw-Hill Professional, 10^{th} ed. 2001); and HANDBOOK OF PHARMACEUTICAL EXCIPIENTS (Raymond C. Rowe et al., APhA Publications, 4^{th} edition 2003) which are hereby incorporated by reference in their entirety. These protocols are routine procedures and any modifications are well within the scope of one skilled in the art and from the teaching herein.

It is further envisioned that a pharmaceutical composition disclosed in the present specification can optionally include, without limitation, other pharmaceutically acceptable components, including, without limitation, buffers, preservatives, tonicity adjusters, salts, antioxidants, physiological substances, pharmacological substances, bulking agents, emulsifying agents, wetting agents, sweetening or flavoring agents, and the like. Various buffers and means for adjusting pH can be used to prepare a pharmaceutical composition disclosed in the present specification, provided that the resulting preparation is pharmaceutically acceptable. Such buffers include, without limitation, acetate buffers, citrate buffers, phosphate buffers, neutral buffered saline, phosphate buffered saline and borate buffers. It is understood that acids or bases can be used to adjust the pH of a composition as needed. Pharmaceutically acceptable antioxidants include, without limitation, sodium metabisulfite, sodium thiosulfate, acetylcysteine, butylated hydroxyanisole and butylated hydroxytoluene. Useful preservatives include, without limitation, benzalkonium chloride, chlorobutanol, thimerosal, phenylmercuric acetate, phenylmercuric nitrate and a stabilized oxy chloro composition, for example, PURITE®. Tonicity adjustors useful in a pharmaceutical composition include, without limitation, salts such as, e.g., sodium chloride, potassium chloride, mannitol or glycerin and other pharmaceutically acceptable tonicity adjustor. The pharmaceutical composition may be provided as a salt and can be formed with many acids, including but not limited to, hydrochloric, sulfuric, acetic, lactic, tartaric, malic, succinic, etc. Salts tend to be more soluble in aqueous or other protonic solvents than are the corresponding free base forms. It is understood that these and other substances known in the art of pharmacology can be included in a pharmaceutical composition useful in the invention.

An anti-BoNT/A composition useful in a method of the invention is administered to a human or other mammal in an effective amount. Such an effective amount generally is the minimum dose necessary to achieve the desired therapeutic effect, which can be, for example, that amount roughly necessary to reduce the symptoms associated with BoNT/A toxicity. For example, the term “effective amount” when used with respect to treating BoNT/A toxicity can be a dose sufficient to the symptoms, for example, by at least 30%, 40%, 50%, 60%, 70%, 80%, 90% or 100%. Such a dose generally is in the range of 0.1-1000 mg/day and can be, for example, in the range of 0.1-500 mg/day, 0.5-500 mg/day, 0.5-100 mg/day, 0.5-50 mg/day, 0.5-20 mg/day, 0.5-10 mg/day or 0.5-5 mg/day, with the actual amount to be administered determined by a physician taking into account the relevant circumstances including the severity of the BoNT/A toxicity, the age and weight of the patient, the patient's general physical condition, the cause of the BoNT/A toxicity and the route of administration. Where repeated administration is used, the frequency of administration depends, in part, on the half-life of the vaccine composition. Suppositories and extended release formulations can be useful in the invention and include, for example, dermal patches, formulations for deposit on or under the skin and formulations for intramuscular injection. It is understood that slow-release formulations also can be useful in the methods of the invention. The subject receiving the vaccine composition can be any mammal or other vertebrate capable of experiencing BoNT/A toxicity, for example, a human, primate, horse, cow, dog, cat or bird.

Various routes of administration can be useful for treating BoNT/A toxicity, according to a method of the invention. A pharmaceutical composition useful in the methods of the invention can be administered to a mammal by any of a variety of means depending, for example, on the type and location of BoNT/A toxicity to be treated, the anti-BoNT/A composition or other compound to be included in the composition, and the history, risk factors and symptoms of the subject. Routes of administration suitable for the methods of the invention include both systemic and local administration. As non-limiting examples, a pharmaceutical composition useful for treating BoNT/A toxicity can be administered orally or by subcutaneous pump; by dermal patch; by intravenous, subcutaneous or intramuscular injection; by topical drops, creams, gels or ointments; as an implanted or injected extended release formulation; as a bioerodible or non-bioerodible delivery system; by subcutaneous minipump or other implanted device; by intrathecal pump or injection; or by epidural injection. An exemplary list of biodegradable polymers and methods of use are described in, e.g., HANDBOOK OF BIODEGRADABLE POLYMERS (Abraham J. Domb et al., eds., Overseas Publishers Association, 1997); CONTROLLED DRUG DELIVERY: DESIGNING TECHNOLOGIES FOR THE FUTURE (Kinam Park & Randy J. Mrsny eds., American Chemical Association, 2000); Vernon G. Wong, Method for Reducing or Preventing Transplant Rejection in the Eye and Intraocular Implants for Use Therefor, U.S. Pat. No. 6,699,493 (Mar. 2, 2004); Vernon G. Wong & Mae W. L. Hu, Methods for Treating Inflammation-mediated Conditions of the Eye, U.S. Pat. No. 6,726,918 (Apr. 27, 2004); David A. Weber et al., Methods and Apparatus for Delivery of Ocular Implants, U.S. Patent Publication No. US2004/0054374 (Mar. 18, 2004); Thierry Nivaggioli et al., Biodegradable Ocular Implant, U.S. Patent Publication No. US2004/0137059 (Jul. 15, 2004), which are hereby incorporated by reference in their entirety. It is understood that the frequency and duration of dosing will be dependent, in part, on the relief desired and the half-life of the BoNT/A toxicity.

In particular embodiments, a method of the invention is practiced by peripheral administration of an anti-BoNT/A composition. As used herein, the term “peripheral administration” or “administered peripherally” means introducing an agent into a subject outside of the central nervous system. Peripheral administration encompasses any route of administration other than direct administration to the spine or brain. As such, it is clear that intrathecal and epidural administration as well as cranial injection or implantation are not within the scope of the term “peripheral administration” or “administered peripherally.”

Peripheral administration can be local or systemic. Local administration results in significantly more of a pharmaceutical composition being delivered to and about the site of local administration than to regions distal to the site of administration. Systemic administration results in delivery of a pharmaceutical composition to essentially the entire peripheral nervous system of the subject and may also result in delivery to the central nervous system depending on the properties of the composition.

Routes of peripheral administration useful in the methods of the invention encompass, without limitation, oral administration, topical administration, intravenous or other injection, and implanted minipumps or other extended release devices or formulations. A pharmaceutical composition useful in the invention can be peripherally administered, for example, orally in any acceptable form such as in a tablet, liquid, capsule, powder, or the like; by intravenous, intraperitoneal, intramuscular, subcutaneous or parenteral injection; by transdermal diffusion or electrophoresis; topically in any acceptable form such as in drops, creams, gels or ointments; and by minipump or other implanted extended release device or formulation.

XI. Method of Reducing Anti-BoNT/A Antibodies

The present invention provides a method of reducing or eliminating botulinum toxin blocking antibodies from a patient by removing blood from a patient; contacting the blood, or an antibody-containing component thereof, with a BoNT/A peptide disclosed in the present invention under conditions suitable for forming a complex of each of the amino acid sequences and anti-botulinum toxin antibody; and removing the complex from the blood or antibody-containing component thereof.

In an embodiment of the present invention, patient blood, or an antibody-containing component thereof, is contacted with a BoNT/A peptide composition having a length of at most 60 amino acids and consisting of at least 5 contiguous amino acids selected from one of the following BoNT/A amino acid sequences: 449-467 of SEQ ID NO:1(N1), 463-481 of SEQ ID NO:1 (N2), 491-509 of SEQ ID NO:1 (N4), 505-523 of SEQ ID NO:1 (N5), 519-537 of SEQ ID NO:1 (N6), 533-551 of SEQ ID NO:1 (N7), 547-565 of SEQ ID NO:1 (N8), 561-579 of SEQ ID NO:1 (N9), 575-593 of SEQ ID NO:1 (N10), 589-607 of SEQ ID NO:1 (N11), 631-649 of SEQ ID NO:1 (N14), 645-663 of SEQ ID NO:1 (N15), 659-677 of SEQ ID NO:1 (N16), 673-691 of SEQ ID NO:1 (N17), 701-719 of SEQ ID NO:1 (N19), 715-733 of SEQ ID NO:1 (N20), 729-747 of SEQ ID NO:1 (N21), 743-761 of SEQ ID NO:1 (N22), 757-775 of SEQ ID NO:1 (N23), 771-789 of SEQ ID NO:1 (N24), 785-803 of SEQ ID NO:1 (N25), 799-817 of SEQ ID NO:1 (N26), 813-831 of SEQ ID NO:1 (N27), 827-845 of SEQ ID NO:1 (N28), 869-887 of SEQ ID NO:1 (C2), 883-901 of SEQ ID NO:1 (C3), 911-929 of SEQ ID NO:1 (C5), 925-943 of SEQ ID NO:1 (C6), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1065-1083 of SEQ ID NO:1 (C16), 1079-1097 of SEQ ID NO:1 (C17), 1107-1125 of SEQ ID NO:1 (C19), 1121-1139 of SEQ ID NO:1 (C20), 1135-1153 of SEQ ID NO:1 (C21), 1149-1167 of SEQ ID NO:1 (C22), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28), 1247-1265 of SEQ ID NO:1 (C29), 1261-1279 of SEQ ID NO:1 (C30), or 1275-1296 of SEQ ID NO:1 (C31), or a conservative variant or immunoreactive fragment thereof, with the proviso that the BoNT/A peptide is not SEQ ID NO:2.

In another embodiment of the present invention, patient blood, or an antibody-containing component thereof, is contacted with a BoNT/A peptide composition having a length of at most 60 amino acids and consisting of at least 5 contiguous amino acids selected from one of the following BoNT/A amino acid sequences: 449-467 of SEQ ID NO:1(N1), 463-481 of SEQ ID NO:1 (N2), 491-509 of SEQ ID NO:1 (N4), 505-523 of SEQ ID NO:1 (N5), 519-537 of SEQ ID NO:1 (N6), 533-551 of SEQ ID NO:1 (N7), 547-565 of SEQ ID NO:1 (N8), 561-579 of SEQ ID NO:1 (N9), 575-593 of SEQ ID NO:1 (N10), 589-607 of SEQ ID NO:1 (N11), 631-649 of SEQ ID NO:1 (N14), 645-663 of SEQ ID NO:1 (N15), 659-677 of SEQ ID NO:1 (N16), 673-691 of SEQ ID NO:1 (N17), 701-719 of SEQ ID NO:1 (N19), 715-733 of SEQ ID NO:1 (N20), 729-747 of SEQ ID NO:1 (N21), 743-761 of SEQ ID NO:1 (N22), 757-775 of SEQ ID NO:1 (N23), 771-789 of SEQ ID NO:1 (N24), 785-803 of SEQ ID NO:1 (N25), 799-817 of SEQ ID NO:1 (N26), 813-831 of SEQ ID NO:1 (N27), 827-845 of SEQ ID NO:1 (N28), 869-887 of SEQ ID NO:1 (C2), 883-901 of SEQ ID NO:1 (C3), 911-929 of SEQ ID NO:1 (C5), 925-943 of SEQ ID NO:1 (C6), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1065-1083 of SEQ ID NO:1 (C16), 1079-1097 of SEQ ID NO:1 (C17), 1107-1125 of SEQ ID NO:1 (C19), 1121-1139 of SEQ ID NO:1 (C20), 1135-1153 of SEQ ID NO:1 (C21), 1149-1167 of SEQ ID NO:1 (C22), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28), 1247-1265 of SEQ ID NO:1 (C29), 1261-1279 of SEQ ID NO:1 (C30), or 1275-1296 of SEQ ID NO:1 (C31). In an aspect of this embodiment, such a BoNT/A peptide is selected from one of the following amino acid sequences: 463-481 of SEQ ID NO:1 (N2), 519-537 of SEQ ID NO:1 (N6), 533-551 of SEQ ID NO:1 (N7), 547-565 of SEQ ID NO:1 (N8), 561-579 of SEQ ID NO:1 (N9), 589-607 of SEQ ID NO:1 (N11), 659-677 of SEQ ID NO:1 (N16), 743-761 of SEQ ID NO:1 (N22), 785-803 of SEQ ID NO:1 (N25), 813-831 of SEQ ID NO:1 (N27), 827-845 of SEQ ID NO:1 (N28), 869-887 of SEQ ID NO:1 (C2), 883-901 of SEQ ID NO:1 (C3), 911-929 of SEQ ID NO:1 (C6), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1121-1139 of SEQ ID NO:1 (C20), 1135-1153 of SEQ ID NO:1 (C21), 1177-1195 of SEQ ID NO:1 (C24), 1261-1279 of SEQ ID NO:1 (C30) or 1275-1296 of SEQ ID NO:1 (C31). In another aspect of this embodiment, such a BoNT/A peptide is selected from one of the following amino acid sequences: 463-481 of SEQ ID NO:1 (N2), 533-551 of SEQ ID NO:1 (N7), 547-565 of SEQ ID NO:1 (N8), 561-579 of SEQ ID NO:1 (N9), 659-677 of SEQ ID NO:1 (N16), 743-761 of SEQ ID NO:1 (N22), 785-803 of SEQ ID NO:1 (N25), 813-831 of SEQ ID NO:1 (N27), 883-901 of SEQ ID NO:1 (C3), 939-957 of SEQ ID NO:l (C7), 967-985 of SEQ ID NO:1 (C9), 981-999 of SEQ ID NO:1 (C1 0), 995-1013 of SEQ ID NO:1 (C11), 1051-1069 of SEQ ID NO:1 (C15), 1121-1139 of SEQ ID NO:1 (C20) or 1177-1195 of SEQ ID NO:1 (C24) or 1275-1296 of SEQ ID NO: 1 (C31). In another aspect of this embodiment, such a BoNT/A peptide is selected from one of the following amino acid sequences: 533-551 of SEQ ID NO: 1 (N7), 547-565 of SEQ ID NO: 1 (N8), 743-761 of SEQ ID NO: 1 (N22), 785-803 of SEQ ID NO: 1 (N25), 813-831 of SEQ ID NO: 1 (N27), 995-1013 of SEQ ID NO: 1 (C11); 1051-1069 of SEQ ID NO: 1 (C15), 1177-1195 of SEQ ID NO: 1 (C24), or 1275-1296 of SEQ ID NO: 1 (C31).

In yet another aspect of this embodiment, patient blood, or an antibody-containing component thereof, is contacted with a BoNT/A peptide composition having a length of at most 60 amino acids and consisting of at least 5 contiguous amino acids selected from one of the following BoNT/A amino acid sequences: 463-481 of SEQ ID NO:1 (N2), 505-523 of SEQ ID NO:1 (N5), 519-537 of SEQ ID NO:1 (N6), 533-551 of SEQ ID NO:1 (N7), 603-621 of SEQ ID NO:1 (N12), 645-663 of SEQ ID NO:1 (N15), 659-677 of SEQ ID NO:1 (N16), 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1079-1097 of SEQ ID NO:1 (C17), 1107-1125 of SEQ ID NO:1 (C19), 1163-1181 of SEQ ID NO:l (C23), 1177-1195 of SEQ ID NO:1 (C24), 1191-1209 of SEQ ID NO:1 (C25), 1233-1251 of SEQ ID NO:1 (C28) or 1275-1296 of SEQ ID NO:1 (C31). In yet another aspect of this embodiment, such a BoNT/A peptide is selected from one of the following amino acid sequences: 533-551 of SEQ ID NO:1 (N7), 659-677 of SEQ ID NO:1 (N16), 701-719 of SEQ ID NO:1 (N19), 729-747 of SEQ ID NO:1 (N21 ), 757-775 of SEQ ID NO:1 (N23), 799-817 of SEQ ID NO:1 (N26), 1065-1083 of SEQ ID NO:1 (C16), 1107-1125 of SEQ ID NO:1 (C19), 1163-1181 of SEQ ID NO:1 (C23), 1191-1209 of SEQ ID NO:1 (C25), 1233-1251 of SEQ ID NO:1 (C28) or 1275-1296 of SEQ ID NO:1 (C31). In yet another aspect of this embodiment, such a BoNT/A peptide is selected from one of the following amino acid sequences: 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1079-1097 of SEQ ID NO:1 (C17), 1107-1125 of SEQ ID NO:1 (C19), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28) or 1275-1296 of SEQ ID NO:1 (C31). In yet another aspect of this embodiment, such a BoNT/A peptide is selected from one of the following amino acid sequences: 729-747 of SEQ ID NO:1 (N21) 799-817 of SEQ ID NO:1 (N26), 1065-1083 of SEQ ID NO:1 (C16), 1163-1181 of SEQ ID NO:1 (C23) or 1275-1296 of SEQ ID NO:1 (C31).

In yet another embodiment of the present invention, patient blood, or an antibody-containing component thereof, is contacted with a BoNT/A peptide composition selected from one of the following BoNT/A amino acid sequences: 449-467 of SEQ ID NO:1(N1), 463-481 of SEQ ID NO:1 (N2), 491-509 of SEQ ID NO:1 (N4), 505-523 of SEQ ID NO:1 (N5), 519-537 of SEQ ID NO:1 (N6), 533-551 of SEQ ID NO:1 (N7), 547-565 of SEQ ID NO:1 (N8), 561-579 of SEQ ID NO:1 (N9), 575-593 of SEQ ID NO:1 (N10), 589-607 of SEQ ID NO:1 (N11), 631-649 of SEQ ID NO:1 (N14), 645-663 of SEQ ID NO:1 (N15), 659-677 of SEQ ID NO:1 (N16), 673-691 of SEQ ID NO:1 (N17), 701-719 of SEQ ID NO:1 (N19), 715-733 of SEQ ID NO:1 (N20), 729-747 of SEQ ID NO:1 (N21), 743-761 of SEQ ID NO:1 (N22), 757-775 of SEQ ID NO:1 (N230, 771-789 of SEQ ID NO:1 (N24), 785-803 of SEQ ID NO:1 (N25), 799-817 of SEQ ID NO:1 (N26), 813-831 of SEQ ID NO:1 (N27), 827-845 of SEQ ID NO:1 (N28), 869-887 of SEQ ID NO:1 (C2), 883-901 of SEQ ID NO:1 (C3), 911-929 of SEQ ID NO:1 (C5), 925-943 of SEQ ID NO:1 (C6), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1065-1083 of SEQ ID NO:1 (C16), 1079-1097 of SEQ ID NO:1 (C17), 1107-1125 of SEQ ID NO:1 (C19), 1121-1139 of SEQ ID NO:1 (C20), 1135-1153 of SEQ ID NO:1 (C21), 1149-1167 of SEQ ID NO:1 (C22), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28), 1247-1265 of SEQ ID NO:1 (C29), 1261-1279 of SEQ ID NO:1 (C30), or 1275-1296 of SEQ ID NO:1 (C31). In an aspect of this embodiment, such a BoNT/A peptide is selected from one of the following amino acid sequences: 463-481 of SEQ ID NO:1 (N2), 519-537 of SEQ ID NO:1 (N6), 533-551 of SEQ ID NO:1 (N7), 547-565 of SEQ ID NO:1 (N8), 561-579 of SEQ ID NO:1 (N9), 589-607 of SEQ ID NO:1 (N11), 659-677 of SEQ ID NO:1 (N16), 743-761 of SEQ ID NO:1 (N22), 785-803 of SEQ ID NO:1 (N25), 813-831 of SEQ ID NO:1 (N27), 827-845 of SEQ ID NO:1 (N28), 869-887 of SEQ ID NO:1 (C2), 883-901 of SEQ ID NO:1 (C3), 911-929 of SEQ ID NO:1 (C6), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1121-1139 of SEQ ID NO:1 (C20), 1135-1153 of SEQ ID NO:1 (C21), 1177-1195 of SEQ ID NO:1 (C24), 1261-1279 of SEQ ID NO:1 (C30) or 1275-1296 of SEQ ID NO:1 (C31). In another aspect of this embodiment, such a BoNT/A peptide is selected from one of the following amino acid sequences: 463-481 of SEQ ID NO:1 (N2), 533-551 of SEQ ID NO:1 (N7), 547-565 of SEQ ID NO:1 (N8), 561-579 of SEQ ID NO:1 (N9), 659-677 of SEQ ID NO:1 (N16), 743-761 of SEQ ID NO:1 (N22), 785-803 of SEQ ID NO:1 (N25), 813-831 of SEQ ID NO:1 (N27), 883-901 of SEQ ID NO:1 (C3), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1121-1139 of SEQ ID NO:1 (C20) or 1177-1195 of SEQ ID NO:1 (C24) or 1275-1296 of SEQ ID NO: 1 (C31). In another aspect of this embodiment, such a BoNT/A peptide is selected from one of the following amino acid sequences: 533-551 of SEQ ID NO: 1 (N7), 547-565 of SEQ ID NO: 1 (N8), 743-761 of SEQ ID NO: 1 (N22), 785-803 of SEQ ID NO: 1 (N25), 813-831 of SEQ ID NO: 1 (N27), 995-1013 of SEQ ID NO: 1 (C11); 1051-1069 of SEQ ID NO: 1 (C15), 1177-1195 of SEQ ID NO: 1 (C24), or 1275-1296 of SEQ ID NO: 1 (C31).

In yet another aspect of this embodiment, patient blood, or an antibody-containing component thereof, is contacted with a BoNT/A peptide composition selected from one of the following BoNT/A amino acid sequences: 463-481 of SEQ ID NO:1 (N2), 505-523 of SEQ ID NO:1 (N5), 519-537 of SEQ ID NO:1 (N6), 533-551 of SEQ ID NO:1 (N7), 603-621 of SEQ ID NO:1 (N12), 645-663 of SEQ ID NO:1 (N15), 659-677 of SEQ ID NO:1 (N16), 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1079-1097 of SEQ ID NO:1 (C17), 1107-1125 of SEQ ID NO:1 (C19), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28) or 1275-1296 of SEQ ID NO:1 (C31). In yet another aspect of this embodiment, such a BoNT/A peptide is selected from one of the following amino acid sequences: 533-551 of SEQ ID NO:1 (N7), 659-677 of SEQ ID NO:1 (N16), 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1107-1125 of SEQ ID NO:l (C19), 1163-1181 of SEQ ID NO:l (C23), 1191-1209 of SEQ ID NO:1 (C25), 1233-1251 of SEQ ID NO:1 (C28) or 1275-1296 of SEQ ID NO:1 (C31). In yet another aspect of this embodiment, such a BoNT/A peptide is selected from one of the following amino acid sequences: 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1079-1097 of SEQ ID NO:1 (C17), 1107-1125 of SEQ ID NO:l (C19), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28) or 1275-1296 of SEQ ID NO:1 (C31). In yet another aspect of this embodiment, such a BoNT/A peptide is selected from one of the following amino acid sequences: 729-747 of SEQ ID NO:1 (N21) 799-817 of SEQ ID NO:1 (N26), 1065-1083 of SEQ ID NO:1 (C16),1163-1181 of SEQ ID NO:1 (C23) or 1275-1296 of SEQ ID NO:1 (C31).

In yet another embodiment of the present invention, patient blood, or an antibody-containing component thereof, is contacted with a BoNT/A peptide composition having a length of at most 60 amino acids and consisting of at least 5 contiguous amino acids selected from one of the following BoNT/A amino acid sequences: 449-467 of SEQ ID NO :1(N1), 463-481 of SEQ ID NO:1 (N2), 491-509 of SEQ ID NO:1 (N4), 505-523 of SEQ ID NO:1 (N5), 519-537 of SEQ ID NO:1 (N6), 533-551 of SEQ ID NO:1 (N7), 547-565 of SEQ ID NO:1 (N8), 561-579 of SEQ ID NO:1 (N9), 575-593 of SEQ ID NO:1 (N10), 589-607 of SEQ ID NO:1 (N11), 631-649 of SEQ ID NO:1 (N14), 645-663 of SEQ ID NO:1 (N15), 659-677 of SEQ ID NO:1 (N16), 673-691 of SEQ ID NO:1 (N17), 701-719 of SEQ ID NO:1 (N19), 715-733 of SEQ ID NO:1 (N20), 729-747 of SEQ ID NO:1 (N21), 743-761 of SEQ ID NO:1 (N22), 757-775 of SEQ ID NO:1 (N23), 771-789 of SEQ ID NO:1 (N24), 785-803 of SEQ ID NO:1 (N25), 799-817 of SEQ ID NO:1 (N26), 813-831 of SEQ ID NO:1 (N27), 827-845 of SEQ ID NO:1 (N28), 869-887 of SEQ ID NO:1 (C2), 883-901 of SEQ ID NO:1 (C3), 911-929 of SEQ ID NO:1 (C5), 925-943 of SEQ ID NO:1 (C6), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:l (C15), 1065-1083 of SEQ ID NO:1 (C16), 1079-1097 of SEQ ID NO:1 (C17), 1107-1125 of SEQ ID NO:1 (C19), 1121-1139 of SEQ ID NO:1 (C20), 1135-1153 of SEQ ID NO:1 (C21), 1149-1167 of SEQ ID NO:1 (C22), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28), 1247-1265 of SEQ ID NO:1 (C29), 1261-1279 of SEQ ID NO:1 (C30), or 1275-1296 of SEQ ID NO:1 (C31), or a conservative variant thereof. In an aspect of this embodiment, such a BoNT/A peptide is selected from one of the following amino acid sequences: 463-481 of SEQ ID NO:1 (N2), 519-537 of SEQ ID NO:1 (N6), 533-551 of SEQ ID NO:1 (N7), 547-565 of SEQ ID NO:1 (N8), 561-579 of SEQ ID NO:1 (N9), 589-607 of SEQ ID NO:1 (N11), 659-677 of SEQ ID NO:1 (N16), 743-761 of SEQ ID NO:1 (N22), 785-803 of SEQ ID NO:1 (N25), 813-831 of SEQ ID NO:1 (N27), 827-845 of SEQ ID NO:1 (N28), 869-887 of SEQ ID NO:1 (C2), 883-901 of SEQ ID NO:1 (C3), 911-929 of SEQ ID NO:1 (C6), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1121-1139 of SEQ ID NO:1 (C20), 1135-1153 of SEQ ID NO:1 (C21), 1177-1195 of SEQ ID NO:1 (C24), 1261-1279 of SEQ ID NO:1 (C30) or 1275-1296 of SEQ ID NO:1 (C31), or a conservative variant thereof. In another aspect of this embodiment, such a BoNT/A peptide is selected from one of the following amino acid sequences: 463-481 of SEQ ID NO:1 (N2), 533-551 of SEQ ID NO:1 (N7), 547-565 of SEQ ID NO:1 (N8), 561-579 of SEQ ID NO:1 (N9), 659-677 of SEQ ID NO:1 (N16), 743-761 of SEQ ID NO:1 (N22), 785-803 of SEQ ID NO:1 (N25), 813-831 of SEQ ID NO:1 (N27), 883-901 of SEQ ID NO:1 (C3), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1121-1139 of SEQ ID NO:1 (C20) or 1177-1195 of SEQ ID NO:1 (C24) or 1275-1296 of SEQ ID NO: 1 (C31), or a conservative variant thereof. In another aspect of this embodiment, such a BoNT/A peptide is selected from one of the following amino acid sequences: 533-551 of SEQ ID NO: 1 (N7), 547-565 of SEQ ID NO: 1 (N8), 743-761 of SEQ ID NO: 1 (N22), 785-803 of SEQ ID NO: 1 (N25), 813-831 of SEQ ID NO: 1 (N27), 995-1013 of SEQ ID NO: 1 (C11); 1051-1069 of SEQ ID NO: 1 (C15), 1177-1195 of SEQ ID NO: 1 (C24), or 1275-1296 of SEQ ID NO: 1 (C31), or a conservative variant thereof.

In yet another aspect of this embodiment, patient blood, or an antibody-containing component thereof, is contacted with a BoNT/A peptide composition having a length of at most 60 amino acids and consisting of at least 5 contiguous amino acids selected from one of the following BoNT/A amino acid sequences: 463-481 of SEQ ID NO:1 (N2), 505-523 of SEQ ID NO:1 (N5), 519-537 of SEQ ID NO:1 (N6), 533-551 of SEQ ID NO:1 (N7), 603-621 of SEQ ID NO:1 (N12), 645-663 of SEQ ID NO:1 (N15), 659-677 of SEQ ID NO:1 (N16), 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1079-1097 of SEQ ID NO:1 (C17), 1107-1125 of SEQ ID NO:1 (C19), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28) or 1275-1296 of SEQ ID NO:1 (C31), or a conservative variant thereof. In yet another aspect of this embodiment, such a BoNT/A peptide is selected from one of the following amino acid sequences: 533-551 of SEQ ID NO:1 (N7), 659-677 of SEQ ID NO:1 (N16), 701-719 of SEQ ID NO:1 (N19), 729-747 of SEQ ID NO:1 (N21), 757-775 of SEQ ID NO:l (N23), 799-817 of SEQ ID NO:1 (N26), 1065-1083 of SEQ ID NO:1 (C16), 1107-1125 of SEQ ID NO:1 (C19), 1163-1181 of SEQ ID NO:1 (C23), 1191-1209 of SEQ ID NO:1 (C25), 1233-1251 of SEQ ID NO:1 (C28) or 1275-1296 of SEQ ID NO:1 (C31), or a conservative variant thereof. In yet another aspect of this embodiment, such a BoNT/A peptide is selected from one of the following amino acid sequences: 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1079-1097 of SEQ ID NO:1 (C17), 1107-1125 of SEQ ID NO:l (C19), 1163-1181 of SEQ ID NO:1 (C23),1177-1195 of SEQ ID NO:1 (C24), 1191-1209 of SEQ ID NO:1 (C25), 1233-1251 of SEQ ID NO:1 (C28) or 1275-1296 of SEQ ID NO:1 (C31), or a conservative variant thereof. In yet another aspect of this embodiment, such a BoNT/A peptide is selected from one of the following amino acid sequences: 729-747 of SEQ ID NO:1 (N21) 799-817 of SEQ ID NO:1 (N26), 1065-1083 of SEQ ID NO:1 (C16), 1163-1181 of SEQ ID NO:1 (C23) or 1275-1296 of SEQ ID NO:1 (C31), or a conservative variant thereof.

In yet another embodiment of the present invention, patient blood, or an antibody-containing component thereof, is contacted with a BoNT/A peptide composition having a length of at most 60 amino acids and consisting of at least 5 contiguous amino acids selected from one of the following BoNT/A amino acid sequences: 449-467 of SEQ ID NO:1(N1), 463-481 of SEQ ID NO:1 (N2), 491-509 of SEQ ID NO:1 (N4), 505-523 of SEQ ID NO:1 (N5), 519-537 of SEQ ID NO:1 (N6), 533-551 of SEQ ID NO:1 (N7), 547-565 of SEQ ID NO:1 (N8), 561-579 of SEQ ID NO:1 (N9), 575-593 of SEQ ID NO:1 (N10), 589-607 of SEQ ID NO:1 (N11), 631-649 of SEQ ID NO:1 (N14), 645-663 of SEQ ID NO:1 (N15), 659-677 of SEQ ID NO:1 (N16), 673-691 of SEQ ID NO:1 (N17), 701-719 of SEQ ID NO:1 (N19), 715-733 of SEQ ID NO:1 (N20), 729-747 of SEQ ID NO:1 (N21), 743-761 of SEQ ID NO:1 (N22), 757-775 of SEQ ID NO:1 (N23), 771-789 of SEQ ID NO:1 (N24), 785-803 of SEQ ID NO:1 (N25), 799-817 of SEQ ID NO:1 (N26), 813-831 of SEQ ID NO:1 (N27), 827-845 of SEQ ID NO:1 (N28), 869-887 of SEQ ID NO:1 (C2), 883-901 of SEQ ID NO:1 (C3), 911-929 of SEQ ID NO:1 (C5), 925-943 of SEQ ID NO:1 (C6), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1065-1083 of SEQ ID NO:1 (C16),1079-1097 of SEQ ID NO:1 (C17),1107-1125 of SEQ ID NO:1 (C19), 1121-1139 of SEQ ID NO:1 (C20), 1135-1153 of SEQ ID NO:1 (C21), 1149-1167 of SEQ ID NO:1 (C22), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28), 1247-1265 of SEQ ID NO:1 (C29), 1261-1279 of SEQ ID NO:1 (C30), or 1275-1296 of SEQ ID NO:1 (C31), or a non-conservative variant thereof. In an aspect of this embodiment, such a BoNT/A peptide is selected from one of the following amino acid sequences: 463-481 of SEQ ID NO:1 (N2), 519-537 of SEQ ID NO:l (N6), 533-551 of SEQ ID NO:1 (N7), 547-565 of SEQ ID NO:1 (N8), 561-579 of SEQ ID NO:1 (N9), 589-607 of SEQ ID NO:1 (N11), 659-677 of SEQ ID NO:l (N16), 743-761 of SEQ ID NO:1 (N22), 785-803 of SEQ ID NO:1 (N25), 813-831 of SEQ ID NO:1 (N27), 827-845 of SEQ ID NO:1 (N28), 869-887 of SEQ ID NO:1 (C2), 883-901 of SEQ ID NO:1 (C3), 911-929 of SEQ ID NO:1 (C6), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1121-1139 of SEQ ID NO:1 (C20), 1135-1153 of SEQ ID NO:1 (C21), 1177-1195 of SEQ ID NO:1 (C24), 1261-1279 of SEQ ID NO:1 (C30) or 1275-1296 of SEQ ID NO:1 (C31), or a non-conservative variant thereof. In another aspect of this embodiment, such a BoNT/A peptide is selected from one of the following amino acid sequences: 463-481 of SEQ ID NO:1 (N2), 533-551 of SEQ ID NO:1 (N7), 547-565 of SEQ ID NO:1 (N8), 561-579 of SEQ ID NO:1 (N9), 659-677 of SEQ ID NO:1 (N16), 743-761 of SEQ ID NO:1 (N22), 785-803 of SEQ ID NO:1 (N25), 813-831 of SEQ ID NO:1 (N27), 883-901 of SEQ ID NO:1 (C3), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1121-1139 of SEQ ID NO:1 (C20) or 1177-1195 of SEQ ID NO:1 (C24) or 1275-1296 of SEQ ID NO: 1 (C31), or a non-conservative variant thereof. In another aspect of this embodiment, such a BoNT/A peptide is selected from one of the following amino acid sequences: 533-551 of SEQ ID NO: 1 (N7), 547-565 of SEQ ID NO: 1 (N8), 743-761 of SEQ ID NO: 1 (N22), 785-803 of SEQ ID NO: 1 (N25), 813-831 of SEQ ID NO: 1 (N27), 995-1013 of SEQ ID NO: 1 (C11); 1051-1069 of SEQ ID NO: 1 (C15), 1177-1195 of SEQ ID NO: 1 (C24), or 1275-1296 of SEQ ID NO: 1 (C31), or a non-conservative variant thereof.

In yet another aspect of this embodiment, patient blood, or an antibody-containing component thereof, is contacted with a BoNT/A peptide composition having a length of at most 60 amino acids and consisting of at least 5 contiguous amino acids selected from one of the following BoNT/A amino acid sequences: 463-481 of SEQ ID NO:1 (N2), 505-523 of SEQ ID NO:1 (N5), 519-537 of SEQ ID NO:1 (N6), 533-551 of SEQ ID NO:1 (N7), 603-621 of SEQ ID NO:1 (N12), 645-663 of SEQ ID NO:1 (N15), 659-677 of SEQ ID NO:1 (N16), 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1079-1097 of SEQ ID NO:1 (C17), 1107-1125 of SEQ ID NO:1 (C19), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28) or 1275-1296 of SEQ ID NO:1 (C31), or a non-conservative variant thereof. In yet another aspect of this embodiment, such a BoNT/A peptide is selected from one of the following amino acid sequences: 533-551 of SEQ ID NO:1 (N7), 659-677 of SEQ ID NO:1 (N16), 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1107-1125 of SEQ ID NO:1 (C19), 1163-1181 of SEQ ID NO:1 (C23), 1191-1209 of SEQ ID NO:1 (C25), 1233-1251 of SEQ ID NO:1 (C28) or 1275-1296 of SEQ ID NO:1 (C31), or a non-conservative variant thereof. In yet another aspect of this embodiment, such a BoNT/A peptide is selected from one of the following amino acid sequences: 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1079-1097 of SEQ ID NO:1 (C17), 1107-1125 of SEQ ID NO:1 (C19), 1163-1181 of SEQ ID NO:1 (C23), 1177-1195 of SEQ ID NO:l (C24), 1191-1209 of SEQ ID NO:l (C25), 1233-1251 of SEQ ID NO:1 (C28) or 1275-1296 of SEQ ID NO:1 (C31), or a non-conservative variant thereof. In yet another aspect of this embodiment, such a BoNT/A peptide is selected from one of the following amino acid sequences: 729-747 of SEQ ID NO:1 (N21) 799-817 of SEQ ID NO:1 (N26), 1065-1083 of SEQ ID NO:1 (C16), 1163-1181 of SEQ ID NO:1 (C23) or 1275-1296 of SEQ ID NO:1 (C31), or a non-conservative variant thereof.

In yet another embodiment of the present invention, patient blood, or an antibody-containing component thereof, is contacted with a BoNT/A peptide composition having a length of at most 60 amino acids and consisting of at least 5 contiguous amino acids selected from one of the following BoNT/A amino acid sequences: 449-467 of SEQ ID NO:1(N1), 463-481 of SEQ ID NO:1 (N2), 491-509 of SEQ ID NO:1 (N4), 505-523 of SEQ ID NO:1 (N5), 519-537 of SEQ ID NO:1 (N6), 533-551 of SEQ ID NO:1 (N7), 547-565 of SEQ ID NO:1 (N8), 561-579 of SEQ ID NO:1 (N9), 575-593 of SEQ ID NO:1 (N10), 589-607 of SEQ ID NO:1 (N11), 631-649 of SEQ ID NO:1 (N14), 645-663 of SEQ ID NO:1 (N15), 659-677 of SEQ ID NO:1 (N16), 673-691 of SEQ ID NO:1 (N17), 701-719 of SEQ ID NO:1 (N19), 715-733 of SEQ ID NO:1 (N20), 729-747 of SEQ ID NO:1 (N21), 743-761 of SEQ ID NO:1 (N22), 757-775 of SEQ ID NO:1 (N23), 771-789 of SEQ ID NO:1 (N24), 785-803 of SEQ ID NO:1 (N25), 799-817 of SEQ ID NO:1 (N26), 813-831 of SEQ ID NO:1 (N27), 827-845 of SEQ ID NO:1 (N28), 869-887 of SEQ ID NO:1 (C2), 883-901 of SEQ ID NO:1 (C3), 911-929 of SEQ ID NO:1 (C5), 925-943 of SEQ ID NO:1 (C6), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1065-1083 of SEQ ID NO:1 (C16), 1079-1097 of SEQ ID NO:1 (C17), 1107-1125 of SEQ ID NO:1 (C19), 1121-1139 of SEQ ID NO:1 (C20), 1135-1153 of SEQ ID NO:1 (C21), 1149-1167 of SEQ ID NO:1 (C22), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28), 1247-1265 of SEQ ID NO:1 (C29), 1261-1279 of SEQ ID NO:1 (C30), or 1275-1296 of SEQ ID NO:1 (C31), or an immunoreactive fragment thereof, with the proviso that the BoNT/A peptide is not SEQ ID NO:2. In an aspect of this embodiment, such a BoNT/A peptide is selected from one of the following amino acid sequences: 463-481 of SEQ ID NO:1 (N2), 519-537 of SEQ ID NO:1 (N6), 533-551 of SEQ ID NO:1 (N7), 547-565 of SEQ ID NO:1 (N8), 561-579 of SEQ ID NO:1 (N9), 589-607 of SEQ ID NO:1 (N11), 659-677 of SEQ ID NO:1 (N16), 743-761 of SEQ ID NO:l (N22), 785-803 of SEQ ID NO:1 (N25), 813-831 of SEQ ID NO:1 (N27), 827-845 of SEQ ID NO:1 (N28), 869-887 of SEQ ID NO:1 (C2), 883-901 of SEQ ID NO:1 (C3), 911-929 of SEQ ID NO:1 (C6), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1121-1139 of SEQ ID NO:1 (C20), 1135-1153 of SEQ ID NO:1 (C21), 1177-1195 of SEQ ID NO:1 (C24), 1261-1279 of SEQ ID NO:1 (C30) or 1275-1296 of SEQ ID NO:1 (C31), or an immunoreactive fragment thereof, with the proviso that the BoNT/A peptide is not SEQ ID NO:2. In another aspect of this embodiment, such a BoNT/A peptide is selected from one of the following amino acid sequences: 463-481 of SEQ ID NO:1 (N2), 533-551 of SEQ ID NO:1 (N7), 547-565 of SEQ ID NO:1 (N8), 561-579 of SEQ ID NO:1 (N9), 659-677 of SEQ ID NO:1 (N16), 743-761 of SEQ ID NO:1 (N22), 785-803 of SEQ ID NO:1 (N25), 813-831 of SEQ ID NO:1 (N27), 883-901 of SEQ ID NO:1 (C3), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1121-1139 of SEQ ID NO:1 (C20) or 1177-1195 of SEQ ID NO:1 (C24) or 1275-1296 of SEQ ID NO: 1 (C31), or an immunoreactive fragment thereof, with the proviso that the BoNT/A peptide is not SEQ ID NO:2. In another aspect of this embodiment, such a BoNT/A peptide is selected from one of the following amino acid sequences: 533-551 of SEQ ID NO: 1 (N7), 547-565 of SEQ ID NO: 1 (N8), 743-761 of SEQ ID NO: 1 (N22), 785-803 of SEQ ID NO: 1 (N25), 813-831 of SEQ ID NO: 1 (N27), 995-1013 of SEQ ID NO: 1 (C11); 1051-1069 of SEQ ID NO: 1 (C15), 1177-1195 of SEQ ID NO: 1 (C24), or 1275-1296 of SEQ ID NO: 1 (C31), or an immunoreactive fragment thereof, with the proviso that the BoNT/A peptide is not SEQ ID NO:2.

In yet another aspect of this embodiment, patient blood, or an antibody-containing component thereof, is contacted with a BoNT/A peptide composition having a length of at most 60 amino acids and consisting of at least 5 contiguous amino acids selected from one of the following BoNT/A amino acid sequences: 463-481 of SEQ ID NO:1 (N2), 505-523 of SEQ ID NO:1 (N5), 519-537 of SEQ ID NO:1 (N6), 533-551 of SEQ ID NO:1 (N7), 603-621 of SEQ ID NO:1 (N12), 645-663 of SEQ ID NO:1 (N15), 659-677 of SEQ ID NO:1 (N16), 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1079-1097 of SEQ ID NO:1 (C17), 1107-1125 of SEQ ID NO:1 (C19), 1163-1181 of SEQ ID NO:l (C23), 1177-1195 of SEQ ID NO:1 (C24), 1191-1209 of SEQ ID NO:1 (C25), 1233-1251 of SEQ ID NO:l (C28) or 1275-1296 of SEQ ID NO:1 (C31), or an immunoreactive fragment thereof, with the proviso that the BoNT/A peptide is not SEQ ID NO:2. In yet another aspect of this embodiment, such a BoNT/A peptide is selected from one of the following amino acid sequences: 533-551 of SEQ ID NO:1 (N7), 659-677 of SEQ ID NO:1 (N16), 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1107-1125 of SEQ ID NO:1 (C19), 1163-1181 of SEQ ID NO:1 (C23), 1191-1209 of SEQ ID NO:1 (C25), 1233-1251 of SEQ ID NO:1 (C28) or 1275-1296 of SEQ ID NO:1 (C31), or an immunoreactive fragment thereof, with the proviso that the BoNT/A peptide is not SEQ ID NO:2. In yet another aspect of this embodiment, such a BoNT/A peptide is selected from one of the following amino acid sequences: 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1079-1097 of SEQ ID NO:1 (C17), 1107-1125 of SEQ ID NO:1 (C19), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28) or 1275-1296 of SEQ ID NO:1 (C31), or an immunoreactive fragment thereof, with the proviso that the BoNT/A peptide is not SEQ ID NO:2. In yet another aspect of this embodiment, such a BoNT/A peptide is selected from one of the following amino acid sequences: 729-747 of SEQ ID NO:1 (N21) 799-817 of SEQ ID NO:1 (N26), 1065-1083 of SEQ ID NO:1 (C16), 1163-1181 of SEQ ID NO:1 (C23) or 1275-1296 of SEQ ID NO:1 (C31), or an immunoreactive fragment thereof, with the proviso that the BoNT/A peptide is not SEQ ID NO:2.

In is envisioned that a BoNT/A peptide useful in a method disclosed in the present specification for contacting patient blood, or an antibody-containing component thereof, can have any of a variety of lengths from at least 5 amino acids to at most 60 amino acids. Therefore, aspects of this embodiment may include a BoNT/A peptide with at least, e.g., five amino acids, six amino acids, seven amino acids, eight amino acids, nine amino acids, ten amino acids, 11 amino acids, 12 amino acids, 13 amino acids, 14 amino acids, 15 amino acids, 16 amino acids, 17 amino acids, 18 amino acids, 19 amino acids, 20 amino acids, 25 amino acids, 30 amino acids, 35 amino acids, 40 amino acids, 45 amino acids, 50 amino acids, 55 amino acids or 60 amino acids. Other aspects of this embodiment may include a BoNT/A peptide with at least, e.g., five amino acids of SEQ ID NO:1, six amino acids of SEQ ID NO:1, seven amino acids of SEQ ID NO:1, eight amino acids of SEQ ID NO:1, nine amino acids of SEQ ID NO:1, ten amino acids of SEQ ID NO:1, 11 amino acids of SEQ ID NO:1, 12 amino acids of SEQ ID NO:1, 13 amino acids of SEQ ID NO:1, 14 amino acids of SEQ ID NO:1, 15 amino acids of SEQ ID NO:1, 16 amino acids of SEQ ID NO:1,17 amino acids of SEQ ID NO:1, 18 amino acids of SEQ ID NO:1, 19 amino acids of SEQ ID NO:1, 20 amino acids of SEQ ID NO:1, 25 amino acids of SEQ ID NO:1, 30 amino acids of SEQ ID NO:1, 35 amino acids of SEQ ID NO:1, 40 amino acids of SEQ ID NO:1, 45 amino acids of SEQ ID NO:1, 50 amino acids of SEQ ID NO:1, 55 amino acids or 60 amino acids of SEQ ID NO:1. In further embodiments, such a BoNT/A peptide of the invention may include a BoNT/A peptide with at least, e.g., five amino acids, six amino acids, seven amino acids, eight amino acids, nine amino acids, ten amino acids, 11 amino acids, 12 amino acids, 13 amino acids, 14 amino acids, 15 amino acids, 16 amino acids, 17 amino acids, 18 amino acids, 19 amino acids, 20 amino acids, 25 amino acids, 30 amino acids, 35 amino acids, 40 amino acids, 45 amino acids, 50 amino acids, 55 amino acids or 60 amino acids and consist of at least 5 contiguous amino acids selected from one of the following BoNT/A amino acid sequences: 449-467 of SEQ ID NO:1(N1), 463-481 of SEQ ID NO:1 (N2), 491-509 of SEQ ID NO:1 (N4), 505-523 of SEQ ID NO:1 (N5), 519-537 of SEQ ID NO:1 (N6), 533-551 of SEQ ID NO:1 (N7), 547-565 of SEQ ID NO:1 (N8), 561-579 of SEQ ID NO:1 (N9), 575-593 of SEQ ID NO:1 (N10), 589-607 of SEQ ID NO:1 (N11), 631-649 of SEQ ID NO:1 (N14), 645-663 of SEQ ID NO:1 (N15), 659-677 of SEQ ID NO:1 (N16), 673-691 of SEQ ID NO:1 (N17), 701-719 of SEQ ID NO:1 (N19), 715-733 of SEQ ID NO:1 (N20), 729-747 of SEQ ID NO:1 (N21), 743-761 of SEQ ID NO:1 (N22), 757-775 of SEQ ID NO:1 (N23), 771-789 of SEQ ID NO:1 (N24), 785-803 of SEQ ID NO:1 (N25), 799-817 of SEQ ID NO:1 (N26), 813-831 of SEQ ID NO:1 (N27), 827-845 of SEQ ID NO:1 (N28), 869-887 of SEQ ID NO:1 (C2), 883-901 of SEQ ID NO:1 (C3), 911-929 of SEQ ID NO:1 (C5), 925-943 of SEQ ID NO:1 (C6), 939-957 of SEQ ID NO:1 (C7), 967-985 of SEQ ID NO:1 (C9), 981-999 of SEQ ID NO:1 (C10), 995-1013 of SEQ ID NO:1 (C11), 1051-1069 of SEQ ID NO:1 (C15), 1065-1083 of SEQ ID NO:1 (C16), 1079-1097 of SEQ ID NO:1 (C17), 1107-1125 of SEQ ID NO:1 (C19), 1121-1139 of SEQ ID NO:1 (C20), 1135-1153 of SEQ ID NO:1 (C21), 1149-1167 of SEQ ID NO:1 (C22), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28), 1247-1265 of SEQ ID NO:1 (C29), 1261-1279 of SEQ ID NO:1 (C30) or 1275-1296 of SEQ ID NO:1 (C31), or a conservative variant, a non-conservative variant, or immunoreactive fragment thereof, with the proviso that the BoNT/A peptide is not SEQ ID NO:2.

In another embodiment of the present invention, a BoNT/A peptide composition useful for contacting patient blood, or an antibody-containing component thereof, in a method disclosed in the present specification can comprise one BoNT/A peptide disclosed in the present specification. In another embodiment of the present invention, a BoNT/A peptide composition useful for contacting patient blood, or an antibody-containing component thereof, in a method disclosed in the present specification can comprise a plurality of BoNT/A peptides disclosed in the present specification. Thus, aspects of this embodiment can include one or more BoNT/A peptides, two or more BoNT/A peptides, three or more BoNT/A peptides, four or more BoNT/A peptides, five or more BoNT/A peptides, six or more BoNT/A peptides, seven or more BoNT/A peptides, eight or more BoNT/A peptides, nine or more BoNT/A peptides, ten or more BoNT/A peptides, 15 or more BoNT/A peptides, 20 or more BoNT/A peptides, 25 or more BoNT/A peptides or 30 or more BoNT/A peptides. In other aspects of this embodiment can include one or more BoNT/A conservative variants, two or more BoNT/A conservative variants, three or more BoNT/A conservative variants, four or more BoNT/A conservative variants, five or more BoNT/A conservative variants, six or more BoNT/A conservative variants, seven or more BoNT/A conservative variants, eight or more BoNT/A conservative variants, nine or more BoNT/A conservative variants, ten or more BoNT/A conservative variants, 15 or more BoNT/A conservative variants, 20 or more BoNT/A conservative variants, 25 or more BoNT/A conservative variants or 30 or more BoNT/A conservative variants. In further aspects of this embodiment can include one or more BoNT/A non-conservative variants, two or more BoNT/A non-conservative variants, three or more BoNT/A non-conservative variants, four or more BoNT/A non-conservative variants, five or more BoNT/A non-conservative variants, six or more BoNT/A non-conservative variants, seven or more BoNT/A non-conservative variants, eight or more BoNT/A non-conservative variants, nine or more BoNT/A non-conservative variants, ten or more BoNT/A non-conservative variants, 15 or more BoNT/A non-conservative variants, 20 or more BoNT/A non-conservative variants, 25 or more BoNT/A non-conservative variants or 30 or more BoNT/A non-conservative variants. In still other aspects of this embodiment can include one or more BoNT/A immunoreactive fragments, two or more BoNT/A immunoreactive fragments, three or more BoNT/A immunoreactive fragments, four or more BoNT/A immunoreactive fragments, five or more BoNT/A immunoreactive fragments, six or more BoNT/A immunoreactive fragments, seven or more BoNT/A immunoreactive fragments, eight or more BoNT/A immunoreactive fragments, nine or more BoNT/A peptides, ten or more BoNT/A immunoreactive fragments, 15 or more BoNT/A immunoreactive fragments, 20 or more BoNT/A immunoreactive fragments, 25 or more BoNT/A immunoreactive fragments or 30 or more BoNT/A immunoreactive fragments. BoNT/A peptides disclosed in the present specification useful for contacting patient blood, or an antibody-containing component thereof, can be selected, for example, depending on immunological factors, such as potency of the peptide in inducing an immune response, and technical factors, such as chemical synthesis yields. It is also understood that the two or more BoNT/A peptides can be provided separately or as part of a compound molecule such as a chimeric peptide or heterologous protein.

In an aspect of this embodiment, a method disclosed in the present specification of contacting patient blood, or an antibody-containing component thereof, uses two or more immunoreactive BoNT/A peptides selected from the following amino acid sequences: 533-551 of SEQ ID NO:1 (N7), 547-565 of SEQ ID NO:1 (N8), 743-761 of SEQ ID NO:1 (N22), 785-803 of SEQ ID NO:1 (N25), 813-831 of SEQ ID NO:1 (N27), 995-1013 of SEQ ID NO:1 (C11); 1051-1069 of SEQ ID NO:1 (C15), 1177-1195 of SEQ ID NO:1 (C24), and 1275-1296 of SEQ ID NO:1 (C31), or a conservative variant, a non-conservative variant or an immunoreactive fragment thereof, with the proviso that the BoNT/A peptide is not SEQ ID NO:2. In another aspect of this embodiment, one of the selected amino acid sequence is 533-551 of SEQ ID NO:1 (N8) or a conservative variant, a non-conservative variant or an immunoreactive fragment thereof, with the proviso that the BoNT/A peptide is not SEQ ID NO:2. In yet another aspect of this embodiment, the following two amino acid sequences are selected: 533-551 of SEQ ID NO:1 (N8) and 981-999 of SEQ ID NO:1 (C10), or a conservative variant, a non-conservative variant or an immunoreactive fragment thereof, with the proviso that the BoNT/A peptide is not SEQ ID NO:2. In yet another aspect of this embodiment, the following three amino acid sequences are selected: 533-551 of SEQ ID NO:1 (N8), 981-999 of SEQ ID NO:1 (C10) and 1051-1069 of SEQ ID NO:1 (C15), or a conservative variant, a non-conservative variant or an immunoreactive fragment thereof, with the proviso that the BoNT/A peptide is not SEQ ID NO:2. In a further aspect of this embodiment, one of the amino acid sequences selected is 785-803 of SEQ ID NO:1 (N25) or a conservative variant, a non-conservative variant or an immunoreactive fragment thereof, with the proviso that the BoNT/A peptide is not SEQ ID NO:2. In a still further aspect of this embodiment, the following two amino acid sequences are selected: 785-803 of SEQ ID NO:1 (N25) and 981-999 of SEQ ID NO:1 (C10), or a conservative variant, a non-conservative variant or an immunoreactive fragment thereof, with the proviso that the BoNT/A peptide is not SEQ ID NO:2. In a still further aspect of this embodiment, the following three amino acid sequences are selected: 785-803 of SEQ ID NO:1 (N25), 981-999 of SEQ ID NO:1 (C10) and 1051-1069 of SEQ ID NO:1 (C15), or a conservative variant, a non-conservative variant or an immunoreactive fragment thereof, with the proviso that the BoNT/A peptide is not SEQ ID NO:2. In an additional aspect of this embodiment, one of the amino acid sequences selected is 813-831 of SEQ ID NO:1 (N27) or a conservative variant, a non-conservative variant or an immunoreactive fragment thereof, with the proviso that the BoNT/A peptide is not SEQ ID NO:2. In another additional aspect of this embodiment, the following two amino acid sequences are selected: 813-831 of SEQ ID NO:1 (N27) and 981-999 of SEQ ID NO:1 (C10), or a conservative variant, a non-conservative variant or an immunoreactive fragment thereof, with the proviso that the BoNT/A peptide is not SEQ ID NO:2. In another additional aspect of this embodiment, the following three amino acid sequences are selected: 813-831 of SEQ ID NO:1 (N27), 981-999 of SEQ ID NO:1 (C10) and 1051-1069 of SEQ ID NO:1 (C15), or a conservative variant, a non-conservative variant or an immunoreactive fragment thereof, with the proviso that the BoNT/A peptide is not SEQ ID NO:2.

In an aspect of this embodiment, a method disclosed in the present specification of contacting patient blood, or an antibody-containing component thereof, uses two or more immunoreactive BoNT/A peptides selected from the following amino acid sequences: 659-677 of SEQ ID NO:1 (N16), 729-747 of SEQ ID NO:1 (N21), 799-817 of SEQ ID NO:1 (N26), 1065-1083 of SEQ ID NO:1 (C16), 1163-1181 of SEQ ID NO:l (C23), and 1275-1296 of SEQ ID NO:1 (C31), or a conservative variant, a non-conservative variant or an immunoreactive fragment thereof, with the proviso that the BoNT/A peptide is not SEQ ID NO:2. In another aspect of this embodiment, one of the amino acid sequences selected is 1065-1083 of SEQ ID NO:1 (C16) or a conservative variant, a non-conservative variant or an immunoreactive fragment thereof, with the proviso that the BoNT/A peptide is not SEQ ID NO:2. In yet another aspect of this embodiment, the following two amino acid sequences are selected: 1065-1083 of SEQ ID NO:1 (C16) and 1163-1181 of SEQ ID NO:1 (C23), or a conservative variant, a non-conservative variant or an immunoreactive fragment thereof, with the proviso that the BoNT/A peptide is not SEQ ID NO:2. In yet another aspect of this embodiment, the following three amino acid sequences are selected: 1065-1083 of SEQ ID NO:1 (C16), 1163-1181 of SEQ ID NO:1 (C23) and 1275-1296 of SEQ ID NO:1 (C31), or a conservative variant, a non-conservative variant or an immunoreactive fragment thereof, with the proviso that the BoNT/A peptide is not SEQ ID NO:2. In a further aspect of this embodiment, one of the amino acid sequences selected is 799-817 of SEQ ID NO:1 (N26) or a conservative variant, a non-conservative variant or an immunoreactive fragment thereof, with the proviso that the BoNT/A peptide is not SEQ ID NO:2. In a still further aspect of this embodiment, the following two amino acid sequences are selected: 799-817 of SEQ ID NO:1 (N26) and 1065-1083 of SEQ ID NO:1 (C16), or a conservative variant, a non-conservative variant or an immunoreactive fragment thereof, with the proviso that the BoNT/A peptide is not SEQ ID NO:2. In a still further aspect of this embodiment, the following three amino acid sequences are selected: 799-817 of SEQ ID NO:1 (N26), 1065-1083 of SEQ ID NO:1 (C16) and 1163-1181 of SEQ ID NO:1 (C23), or a conservative variant, a non-conservative variant or an immunoreactive fragment thereof, with the proviso that the BoNT/A peptide is not SEQ ID NO:2. In an additional aspect of this embodiment, one of the amino acid sequences selected is 729-747 of SEQ ID NO:1 (N21) or a conservative variant, a non-conservative variant or an immunoreactive fragment thereof, with the proviso that the BoNT/A peptide is not SEQ ID NO:2. In another additional aspect of this embodiment, the following two amino acid sequences are selected: 729-747 of SEQ ID NO:1 (N21) and 1065-1083 of SEQ ID NO:1 (C16), or a conservative variant, a non-conservative variant or an immunoreactive fragment thereof, with the proviso that the BoNT/A peptide is not SEQ ID NO:2. In another additional aspect of this embodiment, the following three amino acid sequences are selected: 729-747 of SEQ ID NO:1 (N21),1065-1083 of SEQ ID NO:1 (C16) and 1163-1181 of SEQ ID NO:1 (C23), or a conservative variant, a non-conservative variant or an immunoreactive fragment thereof, with the proviso that the BoNT/A peptide is not SEQ ID NO:2.

It is also envisioned that any and all combinations of BoNT/A peptides disclosed in the specification can be useful for contacting patient blood, or an antibody-containing component thereof, including, e.g., BoNT/A peptides of SEQ ID NO:1, BoNT/A conservative variants, BoNT/A non-conservative variants and BoNT/A immunoreactive fragments. Thus, aspects of this embodiment include one or more BoNT/A peptides comprising one or more BoNT/A peptides of SEQ ID NO: 1 and one or more BoNT/A conservative variants; one or more BoNT/A peptides of SEQ ID NO: 1 and one or more BoNT/A non-conservative variants; one or more BoNT/A peptides of SEQ ID NO: 1 and one or more BoNT/A immunoreactive fragments; one or more BoNT/A conservative variants and one or more BoNT/A non-conservative variants; one or more BoNT/A conservative variants and one or more BoNT/A immunoreactive fragments; one or more BoNT/A non-conservative variants and one or more BoNT/A immunoreactive fragments; one or more BoNT/A peptides of SEQ ID NO: 1, one or more BoNT/A conservative variants and one or more BoNT/A non-conservative variants; one or more BoNT/A peptides of SEQ ID NO: 1, one or more BoNT/A conservative variants and one or more BoNT/A immunoreactive fragments; one or more BoNT/A peptides of SEQ ID NO: 1, one or more BoNT/A non-conservative variants and one or more BoNT/A immunoreactive fragments; one or more BoNT/A conservative variants, one or more BoNT/A non-conservative variants and one or more BoNT/A immunoreactive fragments; or one or more BoNT/A peptides of SEQ ID NO: 1, one or more BoNT/A conservative variants, one or more BoNT/A non-conservative variants and one or more BoNT/A immunoreactive fragments.

The ability of an anti-BoNT/A antibody prepared according to a method of the invention to neutralize the effects of botulinum toxicity on a human or other mammal, and, thus, “protect against” botulinum toxicity, can be determined in an animal model using a variety of methods well known to those skilled in the art. Exemplary animal models of botulism include rodent, rabbit and monkey models of foodborne botulism, rodent and chicken models of infant botulism and rodent models of wound botulism, all of which are described, for example, in Simpson, supra, 1989. It is understood that any of the above methods of removing botulinum toxin blocking antibodies from a patient can be practiced by selectively removing IgG anti-botulinum toxin antibodies. It is further understood that the two or more amino acid sequences can be provided separately or as part of a compound molecule such as a chimeric peptide or heterologous protein.

The BoNT/A peptides disclosed herein also can be useful for therapeutic immunoadsorption for extracorporeal removal of anti-BoNT/A antibodies. Such therapeutic immunoadsorption is well known in the art. In general, blood can be removed from a patient to be treated or having been treated with a botulinum toxin therapeutic such as BOTOX®; and anti-botulinum toxin antibodies subsequently removed from the blood, serum or plasma using affinity chromatography with one or more BoNT/A peptides of the invention are attached to a biocompatible support. In one embodiment, an N25 BoNT/A peptide is used for therapeutic immunoadsorption such that anti-N25 antibodies are removed from patient blood, serum or plasma. In another embodiment, one or a combination of N25, C10, C15, C20 or C31 BoNT/A peptides are used for therapeutic immunoadsorption such that antibodies to epitopes in the peptides used for the immunoadsorption are removed from patient blood, serum or plasma.

Biocompatible solid supports having combinations of two or more BoNT/A peptides can be useful in plasma or other pheresis, or pheresis can be performed using a series of affinity columns or other solid supports each having a different BoNT/A peptide. It is understood that the blood, serum, plasma or lymph are contacted with the one or more BoNT/A peptides attached to a biocompatible solid support under conditions that promote binding between the one or more BoNT/A peptides and anti-botulinum toxin antibodies in the patient fluid. As an example, extracorporeal hemoperfusion can be performed as described in M. Abdul Mazid, Affinity Supports for Hemoperfusion, U.S. Pat. No. 5,149,425 (Sep. 22, 1992). Such conditions can include, without limitation, contact temperatures in the range of 35° C. and 40° C., and contact times of about one to six hours. It is understood that the unbound portion of the blood, plasma, or serum, which is significantly antibody-depleted, is reintegrated with cellular components of whole blood as necessary and reintroduced into the patient on a continuous basis or following collection. One skilled in the art further understands that, if desired, the antibody-depleted blood, plasma or serum can be assayed prior to reintroduction in the patient, for example, using one of the BoNT/A peptide binding assays or protection assays disclosed herein.

Several techniques can be useful for removing anti-BoNT/A antibodies complexed with a BoNT/A peptide. As an example, a solid phase system can utilize a solid phase matrix which is a solid phrase support to which the one or more BoNT/A peptides are bound. The blood, plasma or serum containing the blocking antibodies is passed over the solid support, exiting the solid support and leaving behind the blocking antibody/peptide complexes. A variety of biocompatible solid supports can be useful in the methods of the invention. Such supports are chemically inert with respect to human antibody-containing fluids, have sufficient binding capacity, and generally are in the form of a continuous large surface such as a sheet or column, or in the form of particles or vesicles. Exemplary solid supports useful in the invention, including those useful for affinity chromatography, encompass, without limitation, silica; synthetic silicates such as porous glass, for example, glass fiber filters; biogenic silicates such as diatomaceous earth; silicate-containing materials such as kaolinite and borosilicate; and synthetic polymers such as polystyrene, polyproplene and polysaccharides, see, e.g., A. Heather Good, et al., Methods and Compositions for Attenuating Antibody-mediated Xenograft Rejection in Human Recipients, U.S. Pat. No. 6,607,723 (Aug. 19, 2003); and Mazid, supra, 1992. Biocompatible solid supports useful in the invention further include, yet are not limited to, agarose, which is a neutral linear polysaccharide generally composed of D-galactose and altered 3,6-anhydrogalactose residues, for example, Sepharose (Pharmacia); activated gels, cellulose, nitrocellulose, polyvinylchloride, and diazotized paper. The skilled person understands that these and a variety of other well known biocompatible solid supports can be useful in the methods of the invention.

The one or more BoNT/A peptides can be covalently or noncovalently bound to the solid support using well known methods. Supports which can be non-covalently bound by incubation with the immunosorbent include, without limitation, nitrocellulose, borosilicate, filters, polyvinylchloride, polystyrene and diazotized paper. Activated solid supports such as activated matrices also are well known in the art and commercially available and useful in the invention. Such activated solid supports encompass, without limitation, epoxy-activated agarose; CNBr-activated agarose; 6-aminohexanoic acid and 1,6-diaminohexane-agarose, thiopropyl agarose; carbonyidiimidazole-activated agarose; and aminoethyl and hydrazide-activated polyacrylamide, see, e.g., Daniel R. Henderson et al., Methods of Enhancing Effectiveness of Therapeutic Viral Immunogenic Agent Administration, U.S. Pat. No. 6,406,861 (Jun. 18, 2001; and Joseph P. Balint, Anti-human IGM Immunoadsorbent and Process for Producing Said Immunoadsorbent, U.S. Pat. No. 4,762,787 (Aug. 9,1988).

In one embodiment, the methods of the invention for selectively removing blocking anti-botulinum toxin antibodies are performed using an affinity column. An affinity column is a cylindrical container with filters on both ends which contains a solid support to which the one or more BoNT/A peptides are bound. One skilled in the art understands that plasma or serum generally is passed through a column since whole blood contains cells and particulate matter such as platelets which can impede column flow. In another embodiment, a sheet such as a nitrocellulose sheet is pre-bound with one or more BoNT/A peptides, and blood, plasma or serum is incubated with the immunosorbent-linked nitrocellulose. In a further embodiment, one or more BoNT/A peptides are bound to large polystyrene petri dishes. Blood, plasma or serum from a human or other mammal is incubated with the BoNT/A peptide-linked polystyrene and is decanted, leaving behind the blocking antibodies complexed to the one or more BoNT/A peptides.

It is further understood that pre-clearance of antibodies, or a class of antibody such as the IgG class, can be performed prior to selective removal of anti-botulinum toxin antibodies. From the pre-cleared antibody pool, BoNT/A peptide-reactive antibodies can be selected, and the remaining antibodies reconstituted into the blood to be reperfused into the individual, thus reducing the volume to be passed over the blocking antibody selective support and also reducing non-specific binding. As a non-limiting example, non-specific Protein G Sepharose columns such as PROSORBA® (IMRE; Munich, Germany) or Ig-THERASORB® (Plasmaselect; Teterow, Germany) can be used to remove a significant portion of IgG antibody. A variety of additional techniques suitable for general pre-clearance of antibodies are well known in the art and include, yet are not limited to, ammonium sulfate precipitation with ion exchange chromatography; caprylic acid; DEAE-matrices (ion-exchange chromatography); hydroxyapatite chromatography, and gel filtration (Sepharose), see, e.g., Harlow & Lane, supra, 1998a; and Harlow & Lane, supra, 1998b.

In still a further embodiment, one or more BoNT/A peptides are bound to lipid vesicles, and the lipid vesicle-immunosorbent is mixed with a patient's plasma or serum to allow binding to the blocking antibodies. The plasma or serum is subsequently filtered to remove the lipid vesicle-immunosorbent-antibody complex, see, e.g., James F. Marten, Therapeutic Apheresis, U.S. Pat. No. 4,643,718 (Feb. 17, 1987).

One skilled in the art further understands that one or more BoNT/A peptides of the invention can be used for liquid phase separation of blocking antibodies from patient blood, plasma or serum. Liquid phase separation can be performed, for example, by conjugating one or more BoNT/A peptides to a hapten such as, without limitation, dinitrophenol or fluorescein. After mixing the hapten/BoNT/A peptide conjugate with a patient's blood, plasma or serum, the conjugate forms complexes with anti-botulinum toxin blocking antibodies. As a non-limiting example, such antibody complexes can be precipitated using polyethylene glycol (PEG), and the precipitated complexes separated from the blood, plasma or serum using centrifugation, see, e.g., Paul A. Liberti & Paul Pollara, Selective Removal of Immunospecifically Recognizable Substances from Solution, U.S. Pat. No. 4,551,435 (Nov. 5, 1985). One skilled in the art appreciates that these and other solid-phase and liquid-phase systems can be use5,149d to separate BoNT/A peptide/blocking antibody complexes from patient blood, plasma or serum.

As disclosed herein in Example 9 and discussed above, one or more of the synthetic peptides N25, C10, N15, N20 or N31 binds protective antibodies in the large majority of protective patient sera in a sample of 28 cervical dystonia patients treated with BOTOX® and having MPA-protective sera. Based on this finding, one or more of the BoNT/A peptides N25, C10, N15, N20 or N31, or a conservative variant or immunoreactive fragment thereof, can be useful for decreasing patient non-responsiveness when administered in excess together with a therapeutic botulinum toxin preparation.

The present invention additionally provides a method of predicting or determining immunoresistance to botulinum toxin therapy in a human or other mammal by determining the level of IgG antibodies immunoreactive with the botulinum toxin in the human or other mammal; and comparing the level of IgG antibodies to a control level of IgG antibodies, where an increase in the level of IgG antibodies in the human or other mammal as compared to the control level indicates immunoresistance to the botulinum toxin therapy. Such an increase can be, for example, at least a 5-fold increase or at least a 10-fold increase. In one embodiment, the control level of IgG antibodies is determined in a human or other mammal who has not been treated with botulinum toxin therapy. In another embodiment, the control level of IgG antibodies is determined in a human or other mammal who is responsive to the botulinum toxin therapy. The methods of the invention can be used to predict or determine immunoresistance to any of several botulinum toxin therapies including, without limitation, BoNT/A therapy.

Techniques for determining a level of IgG antibodies immunoreactive with a botulinum toxin such as BoNT/A are well known in the art and are described herein. For example, Example 8 describes a solid-phase radioimmunoassay for IgG anti-BoNT/A antibodies using an anti-mouse IgG secondary antibody. A variety of additional anti-lgG antibodies, including anti-human IgG antibodies, are well known in the art and are commercially available, including, but not limited to, rabbit anti-human IgG from Bethyl Laboratories, Inc. (Montgomery, Tex.) and goat anti-human IgG from Zymed Laboratories, Inc (San Francisco, Calif.). Thus, the methods of the invention can be practiced using any of the immunoassays described hereinabove or well known in the art which are specific for detection of IgG antibodies, for example, through use of an anti-lgG secondary antibody.

It is understood that modifications that do not substantially affect the activity of the various embodiments of this invention are also included within the definition of the invention provided herein. Accordingly, the following examples are intended to illustrate but not limit the present invention.

### EXAMPLES

### Example 1

### Mapping of Human Anti-Pentavalent Botulinum Toxoid Antibodies Using BoNT/A Synthetic Peptides

This example shows antigenic mapping of botulinum toxin A with human anti-BoNT antisera using 29 BoNT/A synthetic peptides that encompass the H_{N} domain of BoNT/A.

Human antisera against BoNT/A were prepared by immunizing human volunteers with a toxoid preparation made from BoNTs A, B, C, D and E as described in Atassi et al. supra, 1996. The binding assays described below were performed using IgG fractions of these antisera. For use as a control, an IgG fraction was prepared using pre-immune human serum.

For use in antigenic mapping, BoNT/A peptides were synthesized, purified and subjected to amino acid analysis by the procedure previously reported, see, e.g., M. Zouhair Atassi et al., Localization and Synthesis of the Hormone-Binding Regions of the Human Thyrotropin Receptor, 88(9) PROC. NATL. ACAD. SCI. USA 3613-3617 (1991). Each peptide was found to have an amino acid composition consistent with that expected from its covalent structure shown in FIG. 1. BoNTs A and B were purchased from Metabiologics, Inc. (Madison, Wis.).

BoNT/A peptides (2.5 μg in 50 μl of PBS) or active BoNT/A (1 μg in 50 μl PBS) were added to the wells of flexible polyvinyl chloride 96-well plates (Becton Dickinson; San Jose, Calif.) and allowed to bind for 18 hours at 4° C. After washing five times with PBS, the plates were blocked for 1 hour at 37° C. with 1% bovine serum albumin (BSA) in PBS. Aliquots (50 μl) of anti-toxin antisera that had been prediluted with 0.1% BSA in PBS (dilutions were human IgG fraction, 1:1000 and 1:2000 (vol/vol)) were pipetted into the appropriate wells and kept at 4° C. for 20 hours. The wells were washed five times with PBS before adding 50 μl of affinity-purified rabbit Ig against human IgG and IgM (Dako Corporation; Carpinteria, Calif.) diluted 1:1000 with 0.1% BSA in PBS to the wells of the plate, and incubating for 2 hours at 37° C.

The wells were then washed five times with PBS, and 50 μl of ¹²⁵I-labeled Protein A (2×10⁵ cpm in 0.1 % BSA in PBS) was distributed to the wells and allowed to incubate for 2 hours at room temperature. Finally, the plates were washed thoroughly to remove unbound radioactivity, the individual wells were cut out and transferred into separate tubes, and bound radioactivity was counted in a gamma-counter (1277 Gamma Master; LKB, Finland). Controls included binding of preimmune or normal sera to BoNT/A and its peptides, as well as binding of immune sera to BSA and unrelated peptides.

Assays were performed in triplicate. Results of the triplicate analyses were expressed as mean of net cpm Å SD, after correction for nonspecific binding in control wells that were coated with BSA and unrelated peptides.

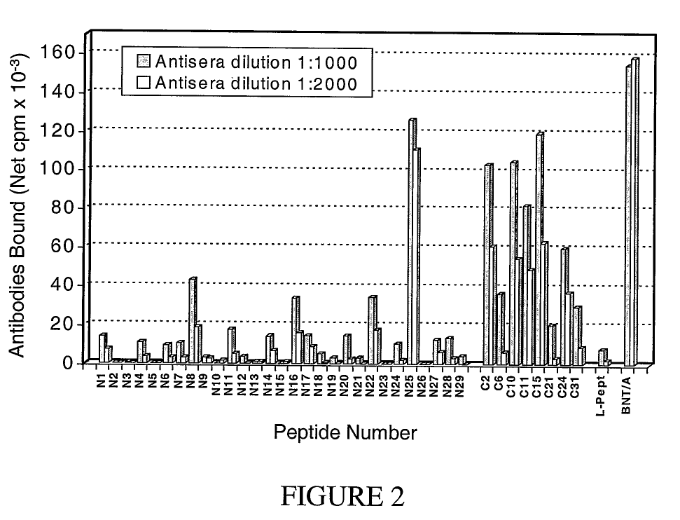

As shown in FIG. 2, human anti-BoNT antisera were observed to bind to several BoNT/A peptides. Peptide N25 (785-803) was observed to be immunodominant followed, in decreasing order, by regions N8 (residues 547-565 of SEQ ID NO:1), N22 (residues 743-761 of SEQ ID NO:1), and N16 (residues 659-677 of SEQ ID NO:1). Lower, but reproducible, amounts of antibodies were bound, in decreasing order, by peptides N11 (residues 589-607 of SEQ ID NO:1), N17 (residues 673-691 of SEQ ID NO:1), N20 (residues 715-733 of SEQ ID NO:1), N14 (residues 631-649 of SEQ ID NO:1), N28 (residues 827-845 of SEQ ID NO:1), N27 (residues 813-831 of SEQ ID NO:1), N4 (residues 491-509 of SEQ ID NO:1), N24 (residues 771-789 of SEQ ID NO:1) and N7 (residues 533-551 of SEQ ID NO:1). The remaining H_{N} peptides bound little or no antibodies. As shown in FIG. 2, human antibodies bound to the H_{C} peptides C2, C6, C10, C11, C1 5, C21, C24, C31 (FIG. 2) in agreement with previous studies, see, e.g., Atassi et al., supra, 1996. Human anti-BoNT antisera exhibited no binding to a control peptide corresponding to amino acids 218-231 of BoNT light chain (“L peptide). Nonimmune human IgG did not bind to any peptides, and human anti-BoNT antisera showed no antibody binding to unrelated proteins and peptides. The results define antigenic portions of the H_{N} domain of BoNT/A.

The three-dimensional structure of BoNT/A reveals the solvent-exposed portions of the primary BoNT/A sequence, D. Borden Lacy et al. Crystal Structure of Botulinum Neurotoxin Type A and Implications for Toxicity, 5(10) NAT. STRUCT. BIOL. 898-902 (1996). Comparison with the results obtained in the present study revealed that the immunodominant antibody-binding regions reside on surface locations on the H subunit of BoNT/A.

In sum, these results demonstrate that BoNT/A peptides N25, N8, N22, N16, N11, N17, N20, N14, N28, N27, N4, N24, N7, C2, C6, C10, C11, C15, C21, C24, and C31 were recognized by human anti-BoNT antisera.

### Example 2

### Mapping of Horse BoNT/A Toxoid Antibodies Using BoNT/A Synthetic Peptides

This example describes antigenic mapping of BoNT/A with horse anti-BoNT antisera using 29 BoNT/A synthetic peptides that encompass the H_{N} domain of BoNT/A.

Horse antisera were prepared by subcutaneous immunization, in multiple sites every two weeks for over a year, with a formaldehyde-inactivated BoNT/A in Ribi adjuvant. The antisera tested in the binding studies were obtained after four injections according to procedures described in Atassi et al., supra, 1996. For use as controls, non-immune horse sera were obtained from the animals before immunization.

Peptide binding assays were performed as described in Example I, except that the dilution for horse antisera was 1:300 (vol/vol). The secondary antibodies were affinity purified rabbit anti-horse IgG obtained from Accurate Chemical & Scientific Corporation (Weston, N.Y.) and were diluted 1:500 (vol/vol).

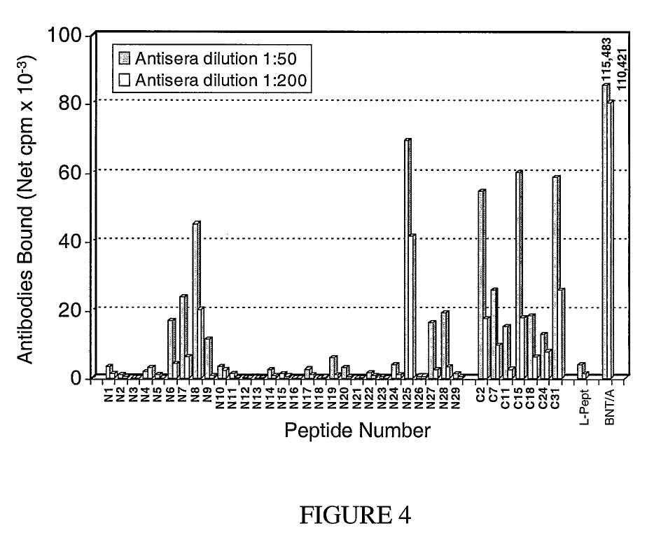

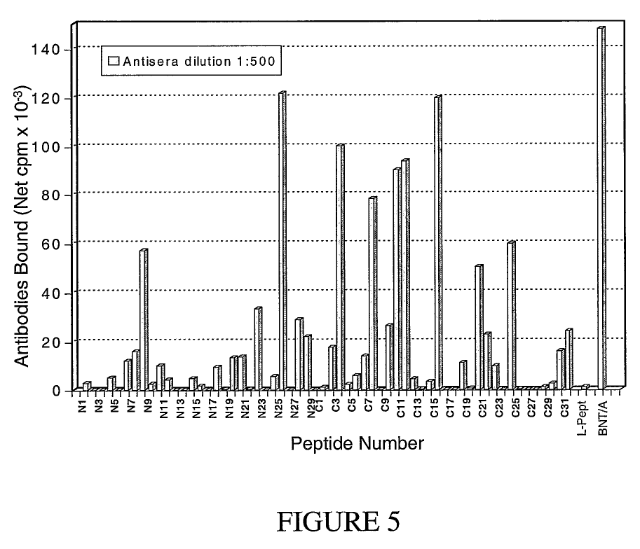

As with the antisera of human, mouse and chicken as described in Examples 1, 2 and 3, one or more regions within the overlapping peptides N7/N8/N9 (residues 533-551/547-565/561-579 of SEQ ID NO:1) were observed to be immunodominant, and peptides N27 (residues 813-831 of SEQ ID NO:1), N25 (residues 785-803 of SEQ ID NO:1), N22 (residues 743-761 of SEQ ID NO:1) and N20 (residues 715-733 of SEQ ID NO:1) possessed binding activity (see FIG. 4). However, horse antibodies exhibited a high level of binding to peptide N2 (residues 463-481 of SEQ ID NO:1), whereas other sera had low levels of binding to peptide N1 (residues 449-467 of SEQ ID NO:1). Therefore, the horse immune response to the BoNT/A region in the vicinity of peptide N2 is shifted to the right by a few residues. The N2 region is also more immunogenic in horse than in human, mouse and chicken. As shown in FIG. 5, horse anti-BoNT antisera were also observed to bind to H_{C} peptides C1, C5, C7, C18, C22, C25, C30 and C31, in agreement with previous studies, see, e.g., Atassi et al., supra, 1996. Using the horse anti-BoNT antisera, no binding to a control peptide corresponding to amino acids 218-231 of BONT light chain was observed. The antisera had no binding to unrelated proteins, and preimmune horse sera bound none of the H_{N} or H_{C} peptides.

In sum, these results demonstrate that peptides N7, N8, N9, N27, N25, N22, N20, N2, N1, C1, C5, C7, C18, C22, C25, C30 and C31 were recognized by horse anti-BoNT antisera.

### Example 3

### Mapping of Mouse Anti-Pentavalent Botulinum Toxoid Antibodies Using BoNT/A Synthetic Peptides

This example describes antigenic mapping of BoNT/A with mouse anti-BoNT antisera using 29 BoNT/A synthetic peptides that encompass the H_{N} domain of BoNT/A.

Mouse anti-BoNT antisera were prepared in outbred ICR mice by subcutaneous immunization with BONT pentavalent toxoid. Antisera used in these studies were obtained 91 days after the first injection, see, e.g., Atassi et al., supra, 1996. Mice were purchased from the National Cancer Institute, and Jackson Laboratory (Bar Harbor, Me.). For use as controls, non-immune mouse sera were obtained from the animals before immunization.

Peptide binding assays were performed as described in Example I, except that the dilution for antisera of outbred mice was 1:50 and 1:200 (vol/vol). The secondary antibodies (mouse IgG (H+L)+IgM (Mu chain) were obtained from Accurate Chemical & Scientific Corporation (Westbury, N.Y.) and were diluted 1:2000 (vol/vol).

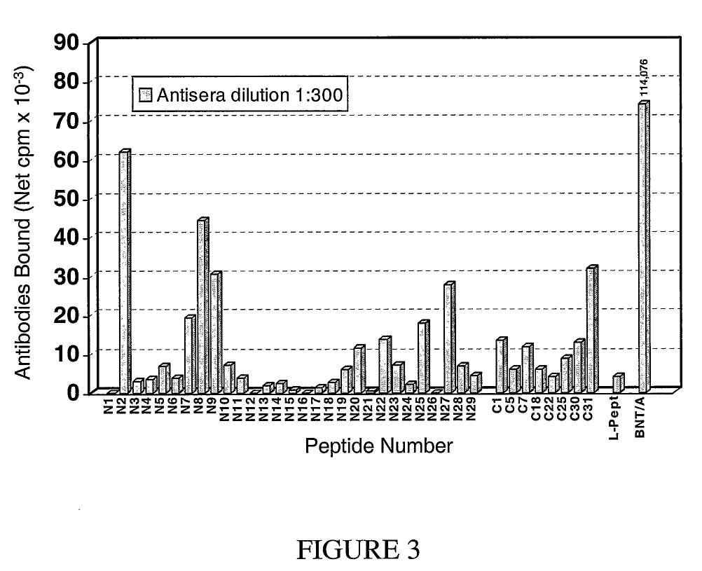

As shown in FIG. 3, mouse anti-BoNT antisera were observed to bind to several BoNT/A peptides. At a dilution of 1:50 (vol/vol), peptide N25 (785-803) was immunodominant, followed by one or more regions within the overlap N6/N7/N8/N9 (residues 519-537/533-551/547-565/561-579 of SEQ ID NO:1) and one or more weaker regions within the overlap N27/N28 (residues 813-831/827-845 of SEQ ID NO:1). At a dilution of 1:200 (vol/vol), peptide N25 (residues 785-803 of SEQ ID NO:1) remained immunodominant; in addition, high amounts of antibodies were bound by the overlap N6/N7/N8 (residues 519-537/533-551/547-565 of SEQ ID NO:1), low amounts of antibodies were bound by the overlap N27/N28 (residues 813-831/827-845 of SEQ ID NO:1), indicating that at least one weak epitope resides within this region (See FIG. 3). As shown in FIG. 3, the H_{C} peptides that possessed antibody binding were C2, C7, C11, C15, C16, C24 and C31, in agreement with previously reported results, see, e.g., Atassi et al., supra, 1996. Mouse anti-BoNT antisera exhibited no binding to a control peptide corresponding to amino acids 218-231 of BoNT light chain (“L peptide”). The mouse anti-BoNT antisera exhibited no antibody binding to unrelated proteins and peptides. Preimmune sera from the same mice did not bind to any of the H_{N} or H_{C} peptides.

In sum, these results demonstrate that peptides N25, N6, N7, N8, N9, N27, N28, C2, C7, C11, C15, C16, C24 and C31 were recognized by mouse anti-BoNT antisera.

### Example 4

### Mapping of Chicken BoNT/A Toxoid Antibodies Using BoNT/A Synthetic Peptides

This example describes antigenic mapping of BoNT/A with chicken anti-BoNT antisera using 29 BoNT/A synthetic peptides that encompass the H_{N} domain of BoNT/A.

Chicken antisera were prepared by monthly subcutaneous injection of formaldehyde-inactivated BoNT/A in Ribi adjuvant. Sera used in this study were obtained after four injections. For use as controls, non-immune chicken sera were obtained from the animals before immunization.

Peptide binding assays were performed as described in Example I, except that the dilution for chicken antisera was 1:500 (vol/vol). The secondary antibodies (rabbit antiserum against chicken IgG) were diluted 1:500 (vol/vol).

As shown in FIG. 4, chicken anti-BoNT antisera were observed to bind to several BoNT/A peptides. In particular, peptide N25 (residues 785-803 of SEQ ID NO:1) was the most immunodominant region, followed by N8 (residues 547-565 of SEQ ID NO:1) (FIG. 4). In addition, lower levels of antibodies were directed, in the following decreasing order of antibody level, against peptides N22 (residues 743-761 of SEQ ID NO:1), N27 (residues 813-831 of SEQ ID NO:1), N28 (residues 827-845 of SEQ ID NO:1), N7 (residues 533-551 of SEQ ID NO:1), N6 (residues 519-537 of SEQ ID NO:1), N19 (residues 701-719 of SEQ ID NO:1) and N20 (residues 715-733 of SEQ ID NO:1). The antibody-binding profile of the peptides corresponding to the entire H chain, including the H_{C} domain is shown in FIG. 4. In the H_{C} domain, chicken antibodies recognized essentially seven major regions, each of which can contain one or more antigenic sites or epitopes. The regions were located within the peptides C15 (residues 1051-1069 of SEQ ID NO:1) and C24 (1177-1195 of SEQ ID NO:1) and the overlaps C2/C3 (residues 869-887/883-901 of SEQ ID NO:1), C6/C7 (residues 925-943/939-957 of SEQ ID NO:1), C9/C10/C11 (residues 967-985/981-999/995-1013 of SEQ ID NO:l), C20/C21/C22 (residue 1121-1139/1135-1153/1149-1167 of SEQ ID NO:l) and C30/C31 (residues 1261-1279/1275-1296 of SEQ ID NO:1). The chicken antisera showed no antibody binding to unrelated proteins and peptides, and chicken anti-BoNT antisera exhibited no binding to a control peptide corresponding to amino acids 218-231 of BoNT light chain. Preimmune chicken sera bound none of the H_{N} or H_{C} peptides.

The binding profile of the chicken anti-BoNT/A antibodies to the panel of H_{C} peptides was similar to that of human antibodies as shown in Table 1. In sum, these results demonstrate that peptides N25, N8 N22, N27, N28, N7, N6, N19, N20, C15, C24,C2, C3, C6, C7, C9, C10, C11, C20, C21, C22, C30, and C31 were recognized by chicken anti-BoNT antisera.

### Example 5

### Comparison of BoNT/A Antiaenicity between Human, Mouse, Chicken and Horse

This example defines several common immunogenic regions of BoNT/A by antigen mapping obtained with antisera from four different species.

The results shown in Examples 1 through 4 indicate that antisera against BoNT/A raised in human, horse, mouse and chicken recognize similar immunodominant regions on the H_{N} domain of BoNT/A. These regions resided, with slight shifts to the left or to the right, within the peptides N6/N7/N8/N9 (residues 519-537/533-551/547-565/561-579 of SEQ ID NO:1) overlap (human, horse and mouse), peptide N22 (residues 743-761 of SEQ ID NO:1) (human, horse and chicken), peptide N25 (residues 785-803 of SEQ ID NO:1) and peptides N27/N28 (residues 813-831/827-845 of SEQ ID NO:1). These results are summarized in Table 2, below.

Whereas peptide N2 was strongly immunodominant with horse antisera, it was unreactive with human, mouse and chicken antisera. However with human, mouse and chicken antisera, peptide N1 reacted weakly and therefore, the reaction of horse antibodies with peptide N2 can represent a shift to the right of the epitope recognized by the horse antibodies. The overlap N16/N17 was highly reactive with human antibodies, whereas with mouse and chicken antisera peptide 17 showed a low level of reactivity. With horse antisera, antibodies against N16/N17 were not detected.

In sum, this example shows that anti-BoNT antibodies from human, mouse, horse and chicken recognize several common immunogenic regions of the BoNT/A H_{N} domain.

TABLE 1

| | Sequence | | | | |
|---|---|---|---|---|---|
| | Position | | | | |
| Peptide | (residues of | | | | |
| No. | SEQ ID NO: 1) | Human | Horse | Mouse | Chicken |
| N1 | 449-467 | ++ | − | + | ± |
| N2 | 463-481 | − | +++++ | − | − |
| N3 | 477-495 | − | ± | − | − |
| N4 | 491-509 | ++ | + | ± | + |
| N5 | 505-523 | − | + | − | − |
| N6 | 519-537 | ++ | + | +++ | ++ |
| N7 | 533-551 | ++ | +++ | +++ | +++ |
| N8 | 547-565 | +++++ | +++++ | +++++ | +++++ |
| N9 | 561-579 | + | ++++ | ++++ | ± |
| N10 | 575-593 | ± | ++ | + | ++ |
| N11 | 589-607 | +++ | + | − | + |
| N12 | 603-621 | + | − | − | − |
| N13 | 617-635 | − | ± | − | − |
| N14 | 631-649 | ++ | ± | ± | + |
| N15 | 645-663 | − | − | − | ± |
| N16 | 659-677 | ++++ | − | − | − |
| N17 | 673-691 | ++ | − | ± | ++ |
| N18 | 687-705 | + | ± | − | − |
| N19 | 701-719 | ± | + | + | ++ |
| N20 | 715-733 | ++ | ++ | ± | ++ |
| N21 | 729-747 | ± | − | − | − |
| N22 | 743-761 | ++++ | ++ | + | ++++ |
| N23 | 757-775 | − | + | − | − |
| N24 | 771-789 | ++ | ± | + | + |
| N25 | 785-803 | +++++ | +++ | +++++ | +++++ |
| N26 | 799-817 | − | − | − | − |
| N27 | 813-831 | ++ | ++++ | +++ | ++++ |
| N28 | 827-845 | ++ | + | +++ | +++ |
| N29 | 841-859 | + | + | − | − |
| L-Peptide | 218-231 | − | − | − | − |
| Active BoNT/A | — | +++++ | +++++ | +++++ | +++++ |

(+) or (−) signs are based on net cpm values and denote the following:
(−), less than 1,500 cpm;
(Å), 1,500-3,000 cpm;
(+), 3,000-7,000 cpm;
(++), 7,000-15,000 cpm;
(+++), 15,000-25,000 cpm;
(++++), 25,000-35,000 cpm;
(+++++), exceeding 35,000 cpm.

TABLE 2

| | Sequence | | | | |
|---|---|---|---|---|---|
| | Position | | | | |
| Peptide | (Residues of | | | | |
| No. | SEQ ID NO: 1) | Human | Horse | Mouse | Chicken |
| C1 | 855-873 | − | +++ | ± | − |
| C2 | 869-887 | +++ | − | +++ | +++ |
| C3 | 883-901 | − | + | + | +++++ |
| C4 | 897-915 | − | ± | − | ± |
| C5 | 911-929 | ++ | + | − | + |
| C6 | 925-943 | +++ | − | − | ++ |
| C7 | 939-957 | + | ++ | + | +++++ |
| C8 | 953-971 | − | − | − | − |
| C9 | 967-985 | + | − | ± | ++++ |
| C10 | 981-999 | +++ | ± | − | +++++ |
| C11 | 995-1013 | +++++ | + | + | +++++ |
| C12 | 1009-1027 | − | − | − | + |
| C13 | 1023-1041 | − | + | − | − |
| C14 | 1037-1055 | − | + | − | + |
| C15 | 1051-1069 | +++++ | ± | ++ | +++++ |
| C16 | 1065-1083 | − | − | − | − |
| C17 | 1079-1097 | − | + | − | − |
| C18 | 1093-1111 | − | + | + | ++ |
| C19 | 1107-1125 | ± | − | − | − |
| C20 | 1121-1139 | + | + | ± | +++++ |
| C21 | 1135-1153 | ++ | ± | ± | +++ |
| C22 | 1149-1167 | ± | + | − | ++ |
| C23 | 1163-1181 | ± | − | − | − |
| C24 | 1177-1195 | +++ | − | ++ | +++++ |
| C25 | 1191-1209 | ± | ++ | − | − |
| C26 | 1205-1223 | − | + | − | − |
| C27 | 1219-1237 | + | − | − | − |
| C28 | 1233-1251 | + | − | − | − |
| C29 | 1247-1265 | ++ | ± | − | ± |
| C30 | 1261-1279 | + | ++ | − | +++ |
| C31 | 1275-1296 | ++ | +++ | ++ | +++ |
| L-Peptide | 218-231 | − | − | − | − |
| Active BoNT/A | — | +++++ | +++++ | +++++ | +++++ |

(+) or (−) signs are based on net cpm values and denote the following:
(−), less than 1,500 cpm;
(Å), 1,500-3,000 cpm;
(+), 3,000-7,000 cpm;
(++), 7,000-15,000 cpm;
(+++), 15,000-25,000 cpm;
(++++), 25,000-35,000 cpm;
(+++++), exceeding 35,000 cpm.

### Example 6

### Identification of Immunodominant Regions of BoNT/A

This example shows the identification of several immunodominant regions of human anti-BoNT antibodies within the H chain of BoNT/A.

The antigenic regions of BONT were determined using anti-BoNT antisera obtained from human, mouse, horse and chicken, as shown in Examples 1 through 4. The location of antigenic regions can be narrowed to shorter domains by the following analysis.

In this analysis, the size of an antigenic site was assigned to be 10-11 residues. The H-chain of BoNT/A was therefore broken down into 13 antigenic sites. The 13 antigenic sites are defined in Table 3, below. The table gives the approximate locations of only the antigenic regions that bind 15,000 cpm of antibody or greater. Although only the immunodominant regions are shown in Table 3, regions binding lower amounts of antibodies can be of equivalent immunological significance.

TABLE 3

| | |
|---|---|
| H_{N} Domain Regions | H_{C} Domain Regions |

| | Amino Acid | | Amino Acid |
|---|---|---|---|
| Antigenic | Residue of | Antigenic | Residue of SEQ |
| Regions | SEQ ID NO: 1 | Regions | ID NO: 1 |
| NR1 | 554-564 | CR1 | 854-887 |
| NR2 | 593-602 | CR2 | 933-943 |
| NR3 | 666-676 | CR3 | 986-995 |
| NR4 | 748-757 | CR4 | 1000-1009 |
| NR5 | 785-794 | CR5 | 1056-1065 |
| | | CR6 | 1137-1147 |
| | | CR7 | 1183-1192 |
| | | CR8 | 1276-1289 |

In sum, this example shows that BoNT/A immunodominant regions having 10-11 residues can be determined based on reactivity of anti-BoNT antisera obtained from human, mouse, horse and chicken with BoNT/A peptides.

### Example 7

### Mapping of T- and B-Cell Recognition Profiles of the BoNT/A H_{N} Domain in Two High-Responder Mouse Strains

This example demonstrates that responses to each antibody or T cell epitope are under separate genetic control and that there is partial, but not complete, coincidence between antibody and T cell H_{N} recognition regions.

A. T Cell Recognition of H_{N} Peptides After One Injection with Toxoid

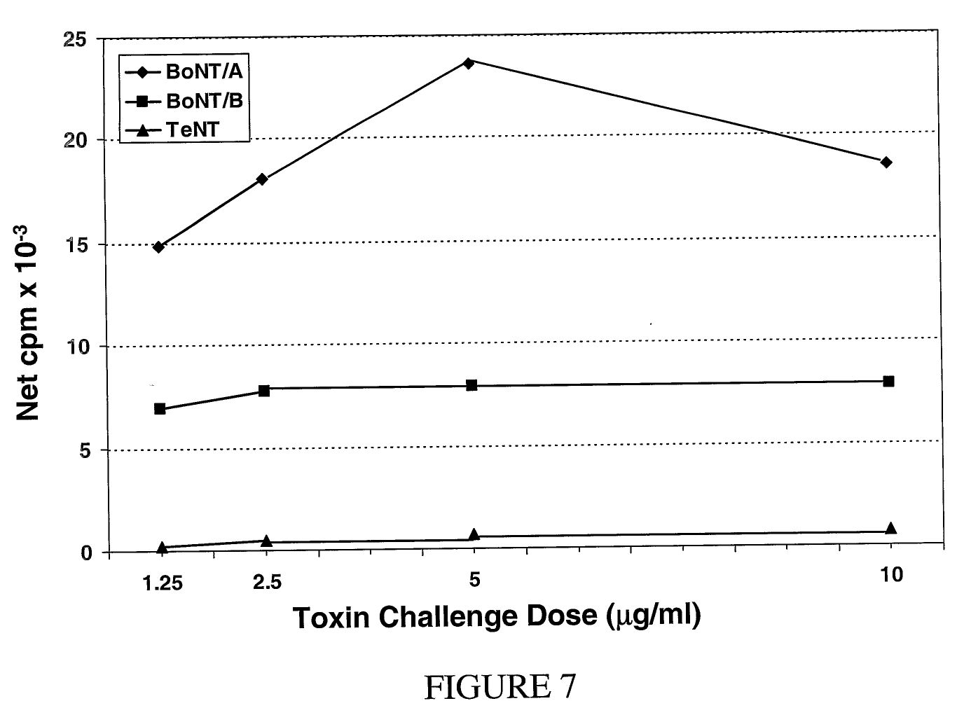

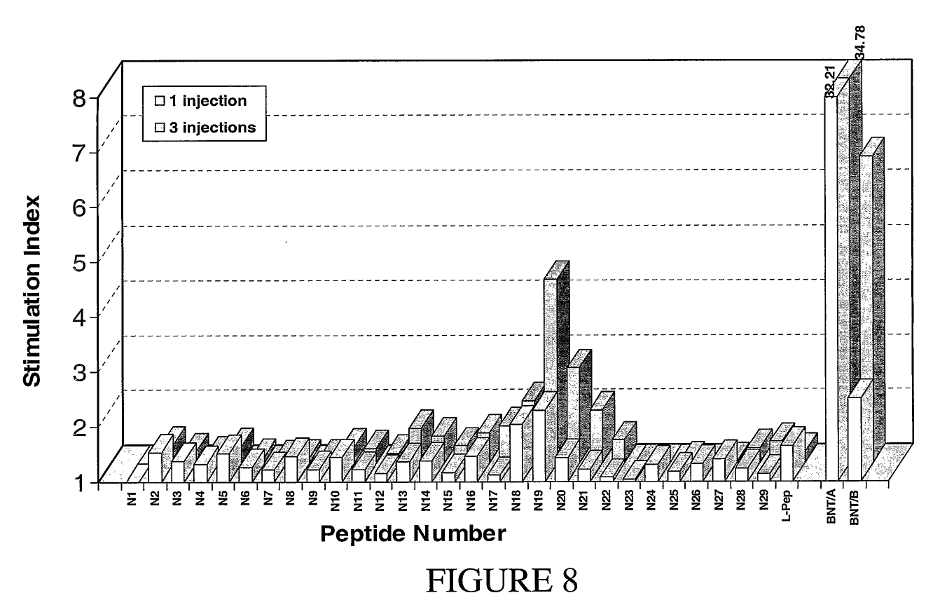

Exemplary proliferative responses of BALB/c lymph node cells (LNCs) were determined at various doses of toxoid as shown in FIG. 7. The response profile to the full panel of H_{N} peptides spanning the entire N-terminal domain of the BoNT/A heavy chain was subsequently determined. As shown in FIG. 8, BALB/c T cells primed with one injection of BoNT/A toxoid recognized one major region localized within overlap N18/N19 (residues 687-705/701-719 of SEQ ID NO: 1) while the remaining peptides had no detectable stimulating activity in vitro. BoNT/A-primed BALB/c T cells showed substantial cross-reaction with BoNT/B (SI values: BoNT/A 23.62, BoNT/B 7.89) but had no cross-reactivity with TeNT (FIG. 7).

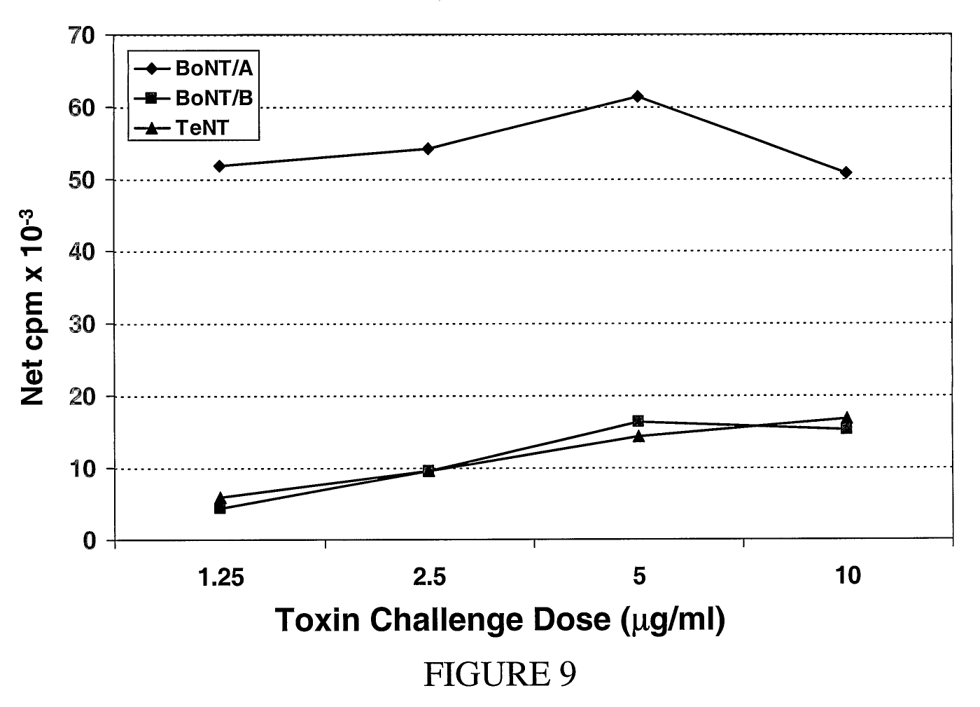

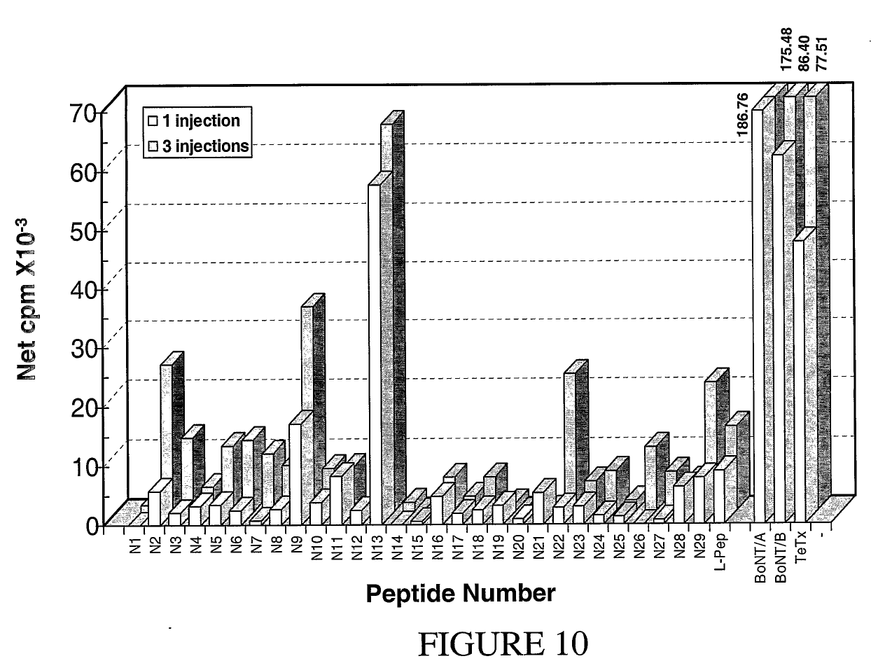

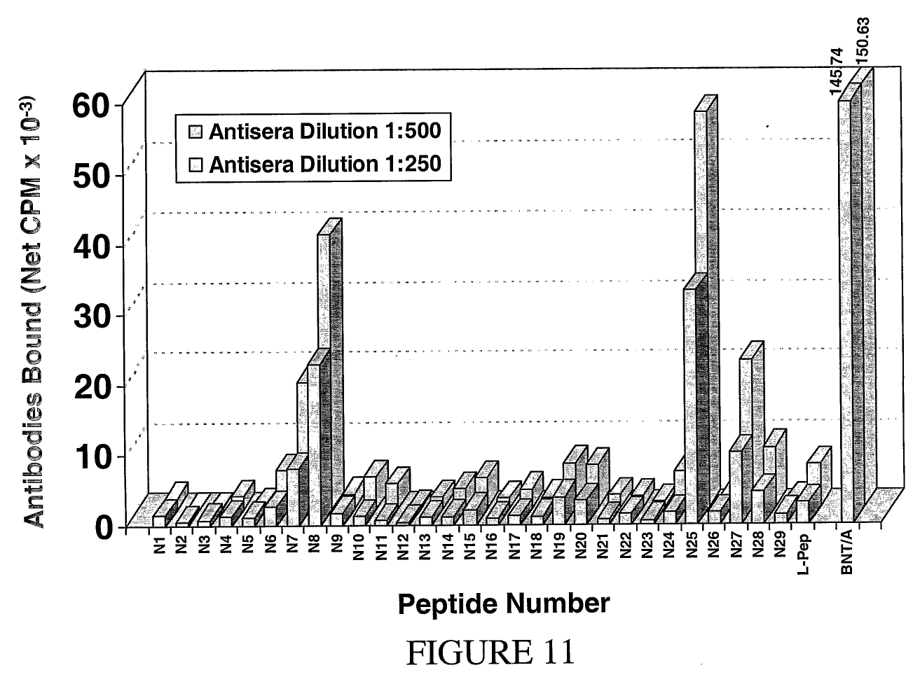

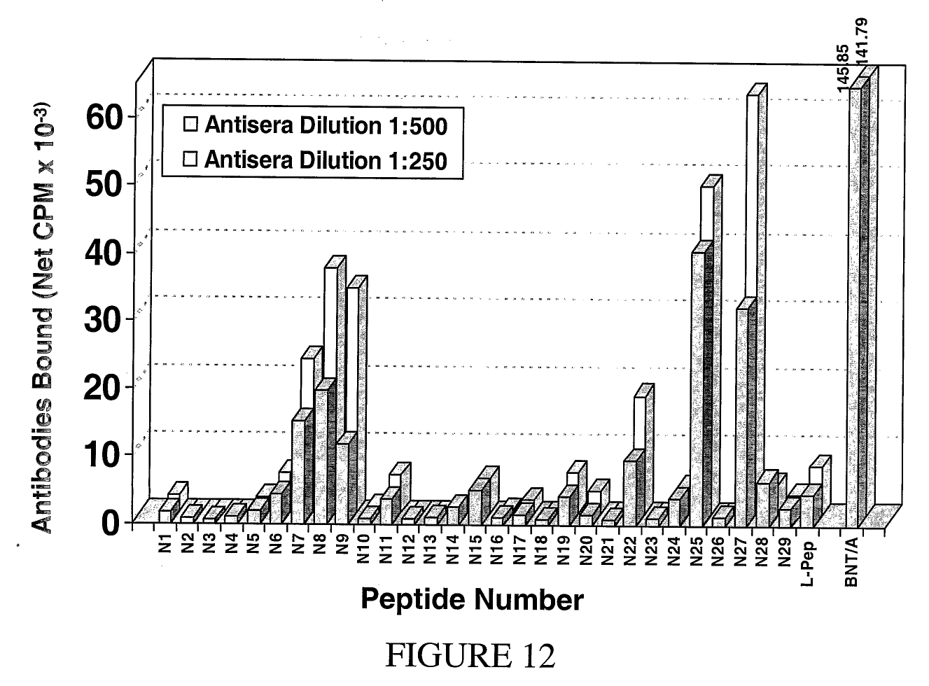

Unlike BALB/c T cells, the T cells from a BoNT/A-primed second inbred strain of mice, SJL/JCr, cross-reacted with both BoNT/B and TeNT (FIG. 9). As summarized in FIG. 10, BoNT/A-primed SJL T cells responded to challenge with a number of the overlapping peptides of H_{N}. In particular, peptides N9 (residues 561-579 of SEQ ID NO: 1), N11 (residues 589-607 of SEQ ID NO: 1), N13 (residues 617-635 of SEQ ID NO: 1), N29 (residues 841-859 of SEQ ID NO: 1) and the L-chain peptide (218-231) stimulated strong-to-medium in vitro T cell responses (SI>5). In addition, peptides N2 (residues 463-481 of SEQ ID NO: 1), N16 (residues 659-677 of SEQ ID NO: 1) and N21 (residues 729-747 of SEQ ID NO: 1) and N28 (residues 827-845 of SEQ ID NO: 1) demonstrated weak (SI>3) stimulating activities. Toxoid-primed T cells of BALB/c and SJL did not respond to the unrelated hen lysozyme or ovalbumin proteins, demonstrating the specificity of the response.

Female BALB/c (H-2^{d}; National Cancer Institute; Frederick, Md.) and SJUJCr (H-2^{s}; (Jackson Laboratory; Bar Harbor, Me.) mice, 7 to 9 weeks old, were used in all experiments. Synthetic peptides were synthesized, purified and characterized as described above. The twenty-nine consecutive overlapping peptides correspond to the complete H_{N} domain (residues 449-859 of SEQ ID NO: 1) and a peptide around the enzymatic active site of the light chain (L-peptide, residues 218-231) of BoNT/A (FIG. 1A). The peptides were 19 residues in length and overlapped consecutively by five residues.

Immunization of mice with BoNT/A toxoid for T cell studies was performed as follows. The optimum priming dose of BoNT/A toxoid was determined in the BALB/c and SJL mouse strains. Mice were immunized subcutaneously at the base of tail with various doses of toxoid (0.125-5 μg/mouse) in a 50-μl emulsion of equal volumes of the toxoid solution in 0.15 M NaCl in 0.01 M sodium phosphate buffer, pH 7.2 (PBS), and complete Freund's adjuvant (CFA) containing Mycobacterium tuberculosis, strain H37Ra (Difco Laboratories; Detroit, Mich.). For both mouse strains, the highest T cell response was obtained at a priming dose of 1 μg/mouse, and subsequent experiments were performed with this dose. The peptides were used in vitro at five doses (5, 10, 20, 40, 80 μg/ml), and the toxin was used in vitro at doses of 1.25, 2.5, 5 and 10 μg/ml.

Lymphocyte proliferation assays were performed as follows. Single-cell suspensions of LNC from toxoid-primed mice were prepared in Hank's balanced salt solution. The cells were washed and resuspended in RPMI 1640 with 1% normal mouse serum and supplemented as described in J. S. Rosenberg et al., Localization of the Regions on the C-Terminal Domain of the Heavy Chain of Botulinum A Recognized by T Lymphocytes and by Antibodies After Immunization of Mice With Pentavalent Toxoid, 26(4) IMMUNOL. INVEST. 491-504 (1997). The number of viable cells was determined by vital staining with fluorescein diacetate. A fixed number of viable LNC (5×10⁵ to 8×10⁵ cells/well) was cocultured in triplicate with various concentrations of mitogen, BoNT/A or synthetic peptides of BoNT/A, BoNT/B or TeNT and control proteins and peptides. The viability of the cells was confirmed in each assay by their responses to ConA and LPS. Negative controls included proteins unrelated to BoNT/A (ovalbumin, myoglobin and hen lysozyme) as well as unrelated control synthetic peptides. After three days of incubation at 37° C. in a humidified, 5% CO₂ atmosphere, lymphocytes were pulsed for 18 hours with [³H]-thymidine (2 μCi/well; Research Products International; Mount Prospect, Ill.) and subsequently harvested onto glass microfiber filters (Whatman; Clinton, N.J.) before counting by liquid scintillation.

B. Mappinci of the T Cell Recognition Profiles After Three Injections with Toxoid

To determine T cell recognition profiles at the time antisera were obtained, proliferative responses were determined for LNC obtained from BALB/c and SJL mice that were used to prepare hyperimmune anti-toxoid antisera for the antibody-binding studies. LNC were harvested at the time of the final bleed on week 10 (i.e. 2 weeks after the last of three injections of toxoid). The proliferative responses to the peptides and toxins of LNC from once-primed and from three-times immunized BALB/c and SJL are shown in FIGS. 8 and 10; the results for both BALB/c and SJL are summarized in Table 4. As shown in FIG. 8, the two recognition profiles for T cells from BALB/c mice were only slightly different (FIG. 8). Hyperimmune T cells responded to challenge in vitro with peptides N18 (residues 687-705 of SEQ ID NO: 1), N19 (residues 701-719 of SEQ ID NO: 1) and N20 (residues 715-733 of SEQ ID NO: 1), with the response to peptide N19 (residues 701-719 of SEQ ID NO: 1) stronger after multiple injections. The recognition profile of the other peptides remained essentially unchanged, and BALB/c hyperimmune T cells did not cross-react with BoNT/B and TeNT.

The recognition profiles of once-primed and of hyperimmune LNC from SJL mice showed greater differences (FIG. 10 and Table 4). As shown in FIG. 10, hyperimmune T cells showed higher cross-reactivity with BoNT/B and TeNT than once-primed cells. In addition, the responses of hyperimmune SJL T cells to peptides N2 (residues 463-481 of SEQ ID NO: 1), N9 (residues 561-579 of SEQ ID NO: 1), N13 (residues 617-635 of SEQ ID NO: 1), N22 (residues 743-761 of SEQ ID NO: 1) and N29 (residues 841-859 of SEQ ID NO: 1) increased markedly. Hyperimmune SJL T cells also responded well to peptides N3 (residues 477-495 of SEQ ID NO: 1), N5 (residues 505-523 of SEQ ID NO: 1), N6 (residues 519-537 of SEQ ID NO: 1), N7 (residues 533-551 of SEQ ID NO: 1), N8 (residues 547-565 of SEQ ID NO: 1), N10 (residues 575-593 of SEQ ID NO: 1), N11 (residues 589-607 of SEQ ID NO: 1), N24 (residues 771-789 of SEQ ID NO: 1), N26 (residues 799-817 of SEQ ID NO: 1), N27 (residues 813-831 of SEQ ID NO: 1) and the L-peptide (218-231).

TABLE 4
T cell recognition regions on BoNT/A H_{N} domain

| | | BALB/c (H-2^{d}) | | SJL (H-2^{S}) | |
|---|---|---|---|---|---|
| | Sequence Position | One | Three | One | Three |
| | (residues of SEQ | Injec- | Injec- | Injec- | Injec- |
| Peptide No. | ID NO: 1) | tion | tions | tion | tions |
| H_{N} Domain | | | | | |
| N1 | 449-467 | − | − | − | − |
| N2 | 463-481 | − | − | + | ++++ |
| N3 | 477-495 | − | − | − | ++ |
| N4 | 491-509 | − | − | − | − |
| N5 | 505-523 | − | − | ± | ++ |
| N6 | 519-537 | − | − | − | ++ |
| N7 | 533-551 | − | − | − | ++ |
| N8 | 547-565 | − | − | − | + |
| N9 | 561-579 | − | − | +++ | ++++ |
| N10 | 575-593 | − | − | ± | + |
| N11 | 589-607 | − | − | ++ | + |
| N12 | 603-621 | − | − | − | − |
| N13 | 617-635 | − | − | +++++ | +++++ |
| N14 | 631-649 | − | − | − | − |
| N15 | 645-663 | − | − | − | − |
| N16 | 659-677 | − | − | + | + |
| N17 | 673-691 | − | − | − | − |
| N18 | 687-705 | ± | ± | − | ± |
| N19 | 701-719 | + | ++ | − | − |
| N20 | 715-733 | − | + | − | − |
| N21 | 729-747 | − | − | + | − |
| N22 | 743-761 | − | − | − | ++++ |
| N23 | 757-775 | − | − | − | ± |
| N24 | 771-789 | − | − | − | + |
| N25 | 785-803 | − | − | − | − |
| N26 | 799-817 | − | − | − | ++ |
| N27 | 813-831 | − | − | − | + |
| N28 | 827-845 | − | − | + | ± |
| N29 | 841-859 | − | − | ++ | +++ |
| Controls | | | | | |
| L-Peptide | 218-231 | − | − | ++ | ++ |
| BoNT/A | — | +++++ | +++++ | +++++ | +++++ |
| BoNT/B | — | + | +++ | +++++ | +++++ |
| TeNT | — | − | − | +++++ | +++++ |

Immunization of mice with BoNT/A toxoid for late T cell responses and antibody binding studies was performed as follows. Mouse antisera were prepared by injection of BALB/c and SJL mice subcutaneously in the hind footpads with 5 μg of toxoid emulsified in complete Freund's adjuvant (CFA). Mice were injected with boosters at 4 and 8 weeks with a similar dose of toxoid, using incomplete Freund's adjuvant (Difco Laboratories; Detroit, Mich.) instead of CFA. Sera were collected prior to the first immunization (pre-immune sera) and two weeks after each injection. For each mouse strain, sera of the respective bleeds from ten mice were pooled and kept at −20° C. until use. Antisera collected on week 10, i.e. 2 weeks after the last injection with toxoid, were employed for peptide binding studies. At the time of the last bleed, lymph nodes were removed, and single cell suspensions prepared for lymphocyte proliferation assays.

C. Binding of Anti-BoNT/A Antibodies to Overlapping Synthetic Peptides and Toxins

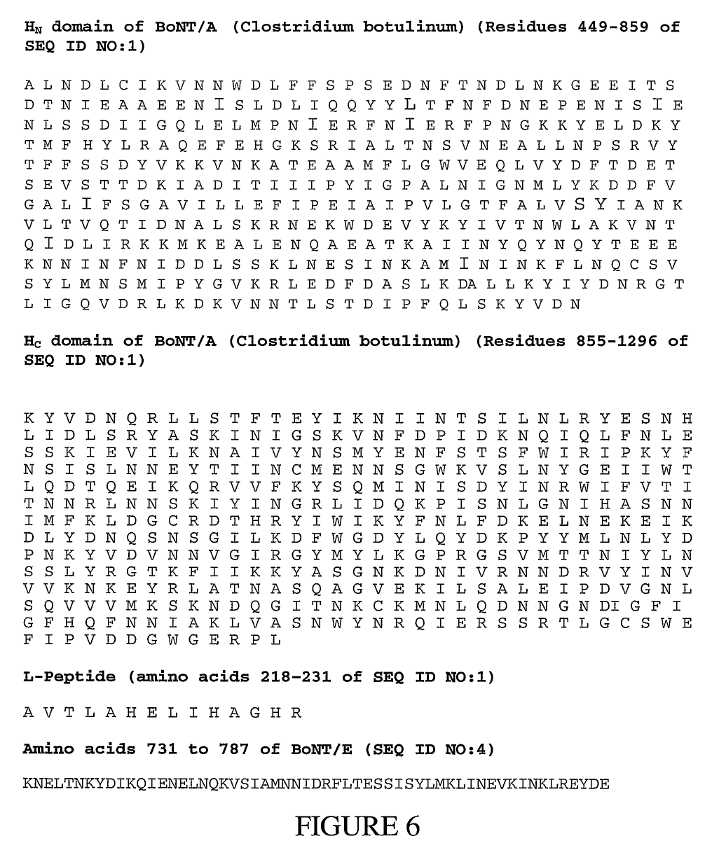

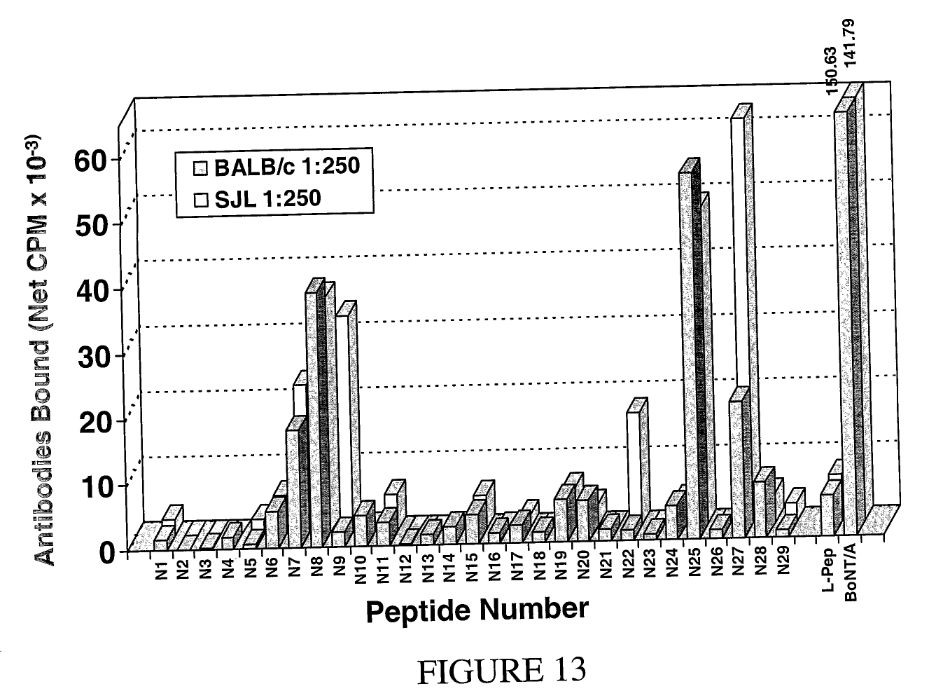

Mapping of antibody binding profiles to peptides in the BALB/c and SLJ inbred mouse strains was performed by assaying antisera at two dilutions (1:250 and 1:500 (vol/vol)). As shown in FIGS. 5 and 6, respectively, the binding profiles of anti-toxoid antibodies from BALB/c and SJL mice were substantially similar. FIG. 13 shows a direct comparison of BALB/c and SJL antisera binding, and Table 5 summarizes the binding profiles for BALB/c and SJL Abs to the H_{N} peptides at a dilution of 1:250 (vol/vol). Antibodies from both mouse strains showed high binding to H_{N} peptides N7, N8, N25 and N27 and low binding to peptides N6, N11, N15 and N19.

Some differences in the binding profiles of antibodies from the two mouse strains were also apparent. In particular, BALB/c antisera showed medium antibody binding to peptide N28 and low antibody binding to peptides N10, N20 and N24, which represented epitopes either unrecognized or poorly recognized by SJL antibodies. On the other hand, SJL antibodies showed high binding to peptides N9 and N22, which were poorly recognized by BALB/c antibodies. In addition, SJL antisera contained much higher amounts of antisera that bound to peptide N27 than did antisera from the other mouse strain. In order to complete the profiles of the H chain recognition by BALB/c and SJL antibodies, Table 5 shows binding profiles to H_{C} peptides previously reported, see, e.g., Rosenberg et al., supra, 1997.

Solid phase radioimmunoassays were performed using Staphylococcal protein A (Pharmacia Biotech; Piscataway, N.J.) radiolabeled with ¹²⁵I (Amersham Corp.; Arlington Heights, Ill.) using the chloramine-T method. Unbound ¹²⁵I was separated from the radiolabeled protein A by gel filtration on a Sephadex G-25 column (0.8×20 cm) equilibrated with PBS containing 0.1% bovine serum albumin (BSA; Sigma Chemicals; St. Louis, Mo.).

Binding of mouse anti-toxoid antibodies to active BoNT/A and synthetic peptides was determined using polyvinylchloride 96-well plates (Becton Dickinson Labware; Oxnard, Calif.), which were coated with each of the 31 overlapping peptides (2.5 μg in 50 μl of PBS/well) or with BoNT/A (1 μg in 50 μl of PBS/well). Wells coated with proteins and synthetic peptides unrelated to BoNTs were used as negative controls. Following overnight incubation at 4° C., plates were washed extensively with PBS and incubated for one hour at 37° C. with 1% BSA in PBS (100 μl/well) to block nonspecific binding in subsequent steps. After washing with PBS, plates were incubated at 37° C. for three hours with mouse antisera (50 μl/well) appropriately prediluted in 0.1% BSA in PBS. For mapping studies, antisera were prediluted 1:250 and 1:500 (vol/vol). Wells were washed with PBS and incubated at 37° C. for two hours with 50 μl of affinity purified rabbit anti-mouse (IgG+IgM) antisera (Accurate Chem. Sci. Corp.; Westbury, N.Y.) pre-diluted 1:1000 (v/v) with 0.1% BSA in PBS. After washing with PBS, ¹²⁵I-labeled protein A was added to the wells (2×10⁵ cpm in 50 μl 0.1% BSA-PBS/well). Plates were subsequently incubated for two hours at room temperature, washed, dried and the wells cut out and counted in a gamma counter (1227 Gammamaster; LKB; Turku, Finland). All determinations were performed in triplicate, and the results expressed as net cpm ±SD, after corrections for nonspecific binding in controls wells that were coated with BSA and of the correlate pre-immune mouse sera to each tested antigen.

TABLE 5
T cells and Abs recognition regions on BoNT/A H_{N} domain^{a}

| | | BALB/ | | SJL | |
|---|---|---|---|---|---|
| Peptide | Sequence Position | c (H-2^{d}) | | (H-2^{S}) | |
| No. | (residues of SEQ ID NO: 1) | Abs | T Cell | Abs | T Cell |
| N1 | 449-467 | ± | − | − | − |
| N2 | 463-481 | − | − | − | ++++ |
| N3 | 477-495 | − | − | − | ++ |
| N4 | 491-509 | ± | − | − | − |
| N5 | 505-523 | − | − | − | ++ |
| N6 | 519-537 | + | − | + | ++ |
| N7 | 533-551 | +++ | − | +++ | ++ |
| N8 | 547-565 | ++++ | − | ++++ | + |
| N9 | 561-579 | ± | − | +++ | ++++ |
| N10 | 575-593 | + | − | − | + |
| N11 | 589-607 | + | − | − | + |
| N12 | 603-621 | − | − | − | − |
| N13 | 617-635 | − | − | − | +++++ |
| N14 | 631-649 | − | − | − | − |
| N15 | 645-663 | + | − | + | − |
| N16 | 659-677 | − | − | − | + |
| N17 | 673-691 | − | − | − | − |
| N18 | 687-705 | − | ± | − | ± |
| N19 | 701-719 | + | ++ | + | − |
| N20 | 715-733 | + | + | − | − |
| N21 | 729-747 | − | − | − | − |
| N22 | 743-761 | ± | − | +++ | ++++ |
| N23 | 757-775 | − | − | − | ± |
| N24 | 771-789 | + | − | − | + |
| N25 | 785-803 | ++++ | − | ++++ | − |
| N26 | 799-817 | − | − | − | ++ |
| N27 | 813-831 | +++ | − | ++++ | + |
| N28 | 827-845 | ++ | − | ± | ± |
| N29 | 841-859 | − | − | − | +++ |
| Controls | | | | | |
| L-Peptide | 218-231 | + | − | + | ++ |
| BoNT/A | — | | +++++ | | +++++ |
| BoNT/B | — | | ++ | | +++++ |
| TeNT | — | | − | | +++++ |
| ^{a}For the purpose of this table, (+) and (−) assignments were based on net cpm values for Ab binding and SI values for T cell proliferation. For Ab binding, the symbols denote the following values: (−), less than 1,500 cpm; (±), 1,500-3,000 cpm; (+), 3,000-7,000 cpm; (++), 7,000-15,000 cpm; (+++), 15,000-25,000 cpm; (++++), 25,000-35,000 cpm; | | | | | |
| # (+++++), exceeding 35,000 cpm. For T cell proliferation, the symbols indicate the following: (−), SI value less then 2.0; (±) 2.0-2.5; (+), SI 2.6-3.5; (++), SI 3.6-6.0; (+++), SI 6.1-10.0; (++++), 10.1-25 (+++++) SI >25.0. | | | | | |

TABLE 6
T cells and Abs recognition regions on BoNT/A H_{c} domain^{a,b}

| | | BALB/ | | SJL | |
|---|---|---|---|---|---|
| Peptide | Sequence Position | c (H-2^{d}) | | (H-2^{S}) | |
| No. | (residues of SEQ ID NO: 1) | Abs | T Cell | Abs | T Cell |
| C1 | 855-873 | − | − | + | − |
| C2 | 869-887 | ++ | − | +++ | − |
| C3 | 883-901 | ++ | − | ++ | − |
| C4 | 897-915 | − | ++ | − | ++++ |
| C5 | 911-929 | − | − | + | + |
| C6 | 925-943 | + | − | + | + |
| C7 | 939-957 | + | ++ | + | +++ |
| C8 | 953-971 | − | − | − | ++ |
| C9 | 967-985 | + | − | + | − |
| C10 | 981-999 | + | − | + | − |
| C11 | 995-1013 | + | − | +++ | − |
| C12 | 1009-1027 | − | + | − | + |
| C13 | 1023-1041 | + | − | − | ++ |
| C14 | 1037-1055 | − | − | − | + |
| C15 | 1051-1069 | + | − | ++ | +++ |
| C16 | 1065-1083 | − | − | − | + |
| C17 | 1079-1097 | − | − | − | ++ |
| C18 | 1093-1111 | − | ± | + | + |
| C19 | 1107-1125 | + | ++ | + | + |
| C20 | 1121-1139 | − | + | + | ++ |
| C21 | 1135-1153 | ++ | − | + | + |
| C22 | 1149-1167 | − | − | + | + |
| C23 | 1163-1181 | − | − | − | ++ |
| C24 | 1177-1195 | − | − | +++ | + |
| C25 | 1191-1209 | − | − | + | + |
| C26 | 1205-1223 | − | − | + | − |
| C27 | 1219-1237 | − | − | − | − |
| C28 | 1233-1251 | − | − | + | + |
| C29 | 1247-1265 | − | − | − | + |
| C30 | 1261-1279 | − | | + | − |
| C31 | 1275-1296 | ++ | − | ++ | ++ |
| Controls | | | | | |
| L-Peptide | 218-231 | + | − | + | ++ |
| BoNT/A | — | | +++++ | | +++++ |
| BoNT/B | — | | ++ | | +++++ |
| TeNT | — | | − | | +++++ |
| ^{a}Results of The Hc domain peptide recognition by anti-toxoid Abs and T cells of BALB/c and SJL mice are from Rosenberg et al., supra, 1997. | | | | | |
| ^{b}For the purpose of this table, (+) and (−) assignments were based on net cpm values for Ab binding and SI values for T cell proliferation. For Ab binding, the symbols denote the following values: (−), less than 1,500 cpm; (±), 1,500-3,000 cpm; (+), 3,000-7,000 cpm; (++), 7,000-15,000 cpm; (+++), 15,000-25,000 cpm; (++++), 25,000-35,000 cpm; | | | | | |
| # (+++++), exceeding 35,000 cpm. For T cell proliferation, the symbols indicate the following: (−), SI value less then 2.0; (±) 2.0-2.5; (+), SI 2.6-3.5; (++), SI 3.6-6.0; (+++), SI 6.1-10.0; (++++), 10.1-25 (+++++) SI >25.0. | | | | | |

D. Protective Activity of Anti-BoNT/A Antibodies In Vivo

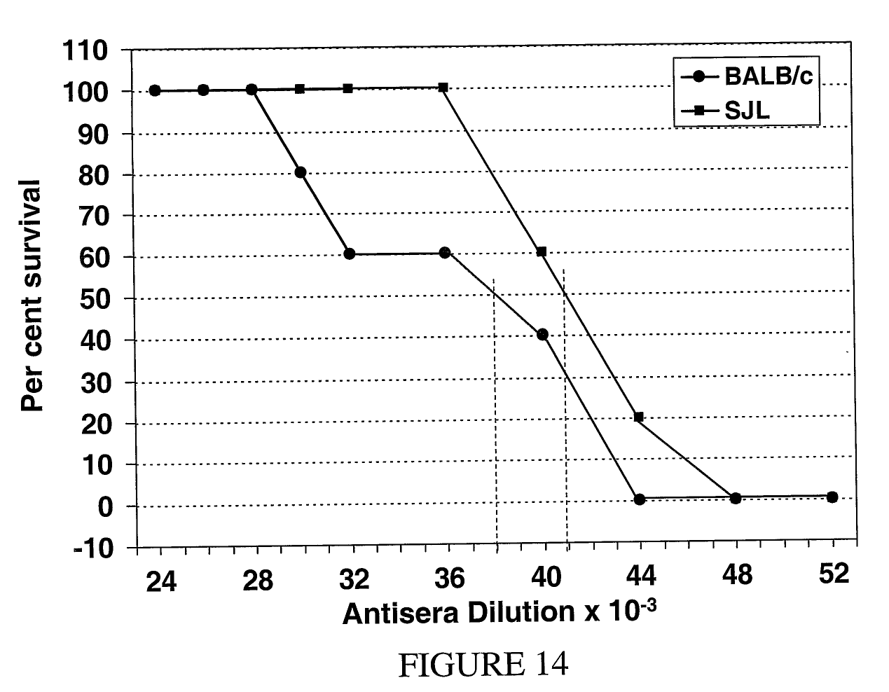

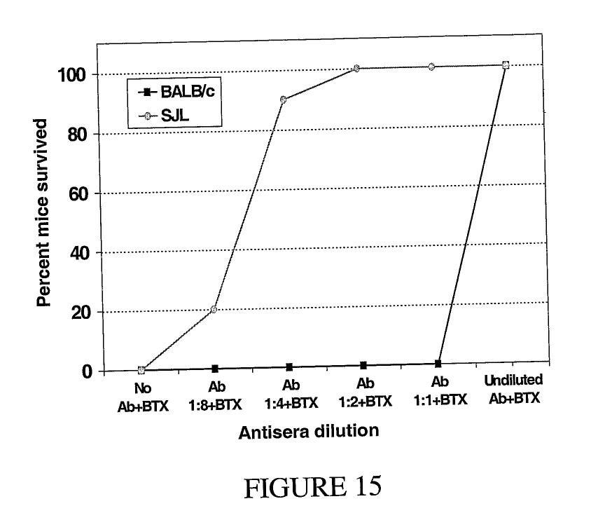

Anti-BoNT/A antisera from BALB/c and SJL mice were assayed for the ability to protect against a lethal dose of active BoNT/A as described further below. Serial dilutions of BALB/c and SJL antisera were assayed for the ability to protect ICR mice against 1.05×LD₁₀₀ (i.e., 6.5 pg) of BoNT/A. As shown in FIG. 14, antisera of both BALB/c and SJL contained high titers of blocking antibodies that protected mice at very high dilutions. Anti-toxin antisera of BALB/c mice were fully protective in recipient ICR mice at dilutions up to 1:28000 (vol/vol), and 50% protection was obtained at 1:38000 (vol/vol). SJL antisera were even more protective, fully protecting recipient ICR mice against a lethal dose of active BoNT/A at 1:36000 dilution (vol/vol), while 50% protection was achieved at 1:41000 dilution (vol/vol). As expected, non-immune sera were not protective at any dilution. These results indicate that anti-toxoid antibodies can be useful for conferring protection against botulinum toxin.

The presence of blocking antibodies in mouse antisera against BoNT/A was determined by a mouse protection assay essentially as follows. The survival of outbred (ICR) mice against various doses of BoNT/A administered intravenously was determined using five mice at each dose. The dose at which no mice survived (i.e., LD₁₀₀) was 5.0 pg/mouse when a fresh preparation of BoNT/A was tested. At the time the mouse protection assays were performed, after storage of toxoid for about 6 months at −20° C. in PBS containing 20% glycerin, the LD₁₀₀ was 6.2 pg/mouse. To determine the protective activity of BALB/c and SJL anti-BoNT/A antisera, ICR mice were injected intravenously in the tail with a mixture of 1.05×LD₁₀₀ of active BoNT/A (i.e., 6.5 pg/mouse) and 100 μl of serial dilutions of the indicated mouse antiserum. Each dilution was injected into five mice, and the mice were observed three times a day for six days. Where test antisera contained blocking antibodies, all mice recovered and survived the challenge. When protecting antibodies were either absent or their amounts too low at high dilution, then none or only some of the mice survived the BoNT/A challenge. The results were plotted as percent survival versus antisera dilutions.

### Example 8

### Submolecular Recognition Profiles in Two Mouse Strains of Non-Protective And Protective Anti-Bont/A Antibodies

This example demonstrates that the switch in BALB/c and SJL mice from non-protective to protective antibodies is not associated with major changes in epitope recognition profiles but is rather associated with the immunoglobulin class of the antibodies.

A. Protective Activity of Anti-BoNT/A Antibodies In Vivo

As described above, female BALB/c (H-2^{d}) and SJL/JCr (H-2^{s}) mice, 7 to 9 weeks old, were used in all experiments. The mouse protection assay was performed as described in Example 7 above. Formaldehyde-inactivated, and active BoNT/A were purchased from Metabiologics (Madison, Wis.).

Anti-toxin antisera of BALB/c and SJL remained unprotective in recipient ICR mice on 26 day after the first BoNT/A injection. Mice were boosted on day 27, and nine days after the second injection (i.e., day 36 after the first injection), antisera were tested for protection. BALB/c antisera were protective against a challenge dose of 1.05×LD₁₀₀, when administered undiluted. SJL antisera were protective on day 36; these antisera were protective at dilutions up to 1:4 and were not protective at dilutions of 1:8. Non-immune sera were not protective even when undiluted. These results serve to define the timing of the switch between production of unprotective and protective anti-BoNT/A antibodies.

B. Binding Profile of Non-Protective and Protective Total Antibodies

For mapping of peptide binding profiles, antisera were assayed at dilutions of 1:100 and 1:250 (vol/vol). Binding profiles of total (IgG and IgM) anti-toxin antibodies from BALB/c and SJL mice were determined for two bleeds: The bleed on day 26 containing non-protective antibodies and the bleed following it on day 36 in which the antibodies demonstrated protective activity.

As shown in FIG. 16, upper panel, non-protective and protective BALB/c antisera showed very similar peptide-binding profiles at a dilution of 1:100. At a dilution of 1:250, the protective BALB/c antisera displayed higher binding to essentially every peptide (FIG. 16, lower panel). The BALB/c antibody-binding peptides were: N6, N7, N25, C2, C3, C9, C10, C11, C15, C18, C24, C30 and C31. Antibodies in the non-protective and protective antisera bound to peptide C30 at similar levels at a dilution of 1:100. However, at a dilution of 1:250, antibody binding to C30 in the non-protective antisera was greatly diminished while binding in the protective antisera remained unaffected, indicating a lower affinity of the antibodies directed against region C30 in the non-protective antisera. Low, but reproducible amounts of antibodies were bound by peptides N19, C6, C7 and C28.

The binding profiles for SJL total antibodies are shown in FIG. 17 at dilutions of 1:100 and 1:250 (vol/vol), upper and lower panels, respectively. In the case of the SJL mice, some differences were apparent between non-protective and protective antisera when total antibodies were analyzed. Peptides N5, N22 and C21, which were recognized by protective antisera, were only slightly recognized (N22 and C21) or not recognized (N5) by non-protective sera. Additionally, in the protective antisera, peptides N7, N8, N25, C11, C15 and less so N27, N28 bound two-fold or higher amounts of antibodies as compared with non-protective antisera. Additional peptides, C4 and C29, bound higher amounts of antibodies in protective sera as compared to non-protective sera at a dilution of 1:100. However these differences disappeared at 1:250, indicating that these antibodies were of relatively low affinity. Peptides C2, C3, C7, C18, C19, C24, C30 and C31 also bound higher amounts of antibodies in protective sera as compared with non-protective antisera, but the differences were less than double. As expected, anti-toxin antibodies did not bind to unrelated proteins or peptides, and pre-immune sera displayed no binding to BoNT/A or its peptides, indicative of specific binding.

In sum, these results demonstrate only very small differences between the peptide recognition profiles of protective and non-protective antisera. These results further indicate that differences in antibody binding levels likely do not account for the difference in protective activity of the non-protective and protective antisera. (0386] Assays were performed as follows. A total of 60 consecutive overlapping peptides corresponding to the complete H subunit (residues 449-1296 of SEQ ID NO: 1), and a peptide around the enzymatic active site of the light chain (L-peptide, residues 218-231), of BoNT/A (FIG. 1) were synthesized, purified and characterized as described above. The peptides were 19 residues long and overlapped consecutively by five residues except for the last peptide in the sequence (C31, residues 1275-1296 of SEQ ID NO: 1). Mice were immunized as described above, with two boosters given at days 27 and 60 with a similar dose of toxoid, using incomplete Freund's adjuvant. Sera were collected prior to the first immunization (preimmune sera) and on days 20, 26, 36, 46, 57, 68 and 70. For each mouse strain, sera of the respective bleeds from 10 mice were pooled and kept at −20° C. The non-protective sera from day 26 and protective sera from day 36 were employed for peptide binding studies. Binding was determined by solid-phase radioimmunoassay as described in Example 7 above, except that affinity-purified rabbit anti-mouse (IgG and IgM) or anti-mouse IgG antisera (Accurate Chem. Sci. Corp.; Westbury, N.Y.) was used as appropriate.

C. Binding of Non-Protective and Protective IgG Antibodies to Synthetic BoNT/A Peptides and to BoNT/A

As described above, differences in total antibody reactivity between protective and non-protective antisera, particularly in the case of BALB/c antisera, appeared insufficient to explain the protective properties of the antisera. The peptide-binding profiles of IgG antibodies alone showed different results. In their binding to active BoNT/A, BALB/c and SJL protective antisera had 13-36 fold higher levels of lgG antibodies relative to non-protective antisera. The profiles for BALB/c and SJL protective and non-protective antibodies are shown in FIGS. 18 and 19, respectively. IgG antibodies in the protective antisera of each mouse strain bound to the same peptides as did total antibodies (IgG and IgM) in the correlate antiserum. However, in both mouse strains, the non-protective antisera contained few, if any, IgG antibodies that bound to these peptides, even at a dilution of 1:100. Again, specific binding was demonstrated by the absence of binding to unrelated proteins and peptides, and by the absence of BoNT/A binding by non-immune sera.

These results demonstrate that protective antibodies had much higher IgG levels that bound to BoNT/A and to synthetic BoNT/A peptides (FIG. 18). In their binding to active BoNT/A, BALB/c protective antisera had up to 36-fold higher amounts of IgG antibodies relative to non-protective antisera (FIG. 18). Similarly, for SJL, the protective antibodies had up to 16-fold higher levels of IgG that bound to active BoNT/A than did the non-protective antibodies (FIG. 19). Furthermore, non-protective SJL and BALB/c antibodies each exhibited little or no binding to the peptides. These results demonstrate that the major difference between the protective and non-protective antibodies was the fact that non-protective antibodies, obtained after only one immunization with BoNT/A, were primarily of the IgM class. In contrast, protective antibodies obtained 10 days after the first booster displayed an IgM-to-IgG switch. In sum, these results indicate that protection is associated with antibodies of the IgG class.

### Example 9

### Mapping of the H Chain Recognition Profile in Antisera From a Cohort of Cervical Dystonia (CD) Patients

This example demonstrates that an in vitro assay can be used to determine amounts of blocking or protective antibodies against BoNT/A in human serum samples. This example further demonstrates that a combination assay using, for example, two or three synthetic BoNT/A peptides can be used for sensitive detection of the presence of specific anti-toxin antibodies in, for example, BOTOX® treated patients.

A. Methods for Data Analysis

MPA-positive cervical dystonia (CD) serum samples were obtained from Allergan, Parkinson's Disease Center and Movement Disorders Clinic of Baylor College of Medicine, and the Arizona Dystonia Institute. CD patient sera protected against a lethal dose of BoNT/A in a mouse protection bioassay were screened with 60 synthetic toxin peptides corresponding to the entire H chain of BoNT/A (FIG. 1). The IgG fraction of hyperimmune sera of human volunteers (obtained from the Department of the Army) against pentavalent toxin (BoNT/A, B, C, D and E) was used as a positive control. An aliquot (50 μl) of each of the 60 synthetic overlapping peptides, dissolved in 0.01 M phosphate buffer, pH 7.2 containing 0.15 M NaCl (1.0 μg/50 μl of PBS), was added to three wells of a flexible polyvinyl chloride 96-well plate. Peptides were allowed to bind for two hours at 37° C. followed by overnight incubation at 4° C. Plates were washed five times with PBS to remove unbound peptide and then blocked for one hour at 37° C. with 0.5% bovine serum albumin in PBS (BSA/PBS). An appropriate volume of each of the mouse protection assay (MPA)-positive CD sera was preincubated with an equal volume of TeNT toxoid (1 mg/ml) for three hours at 37° C. after which it was diluted to 1:500 (vol/vol) with 0.1% BSA/PBS, pipefted (50 μl) into peptide-coated wells and allowed to reactforthree hours at 37° C. followed by further incubation overnight at 4° C. After washing the wells five times with PBS, 50 μl of prediluted (1:500 vol/vol, in 0.1% BSA/PBS) immunoglobulin fraction of rabbit anti-human IgG (DAKO Corporation; Carpinteria, Calif. A0424)+IgM (Mu chain; DAKO, A0426) was added and allowed to react at 37° C. for two hours. The wells were washed five times with PBS followed by addition of 50 μl of ¹²⁵I-Protein A (2×10⁵ CPM in 0.1% BSA/PBS) to each well and incubation for two hours at room temperature. Finally, plates were washed thoroughly to eliminate unbound radioactivity; individual wells were cut out and transferred into separate tubes; and the incorporated radioactivity was counted in a gamma-counter (1277 Gamma Master; LKB, Finland). The results, which were obtained from triplicate analyses, were expressed as the ratios of mean CPM bound by peptides over CPM bound by control peptides or bovine serum albumin (BSA).

For determining antibody binding to BoNT/A or BoNT/B, triplicate wells were coated with the appropriate inactive BoNT/A or BoNT/B toxin (0.5 μg/50 μl of PBS). A similar procedure was then used to determine the amount of antibody bound by BoNT/A or BoNT/B using human MPA-positive CD sera pre-absorbed with TeNT.

B. Assay of Total Antibodies Bound to BoNT/A and BoNT/B

Due to varying amounts of anti-TeNT antibodies in human sera and the cross-reactivity of these antibodies with both BoNT/A as well as BoNT/B, see, e.g., Behzod Z. Dolimbek et al., Cross Reaction of Tetanus and Botulinum Neurotoxins A and B and the Boosting Effect of Botulinum Neurotoxins A and B on a Primary Anti-Tetanus Antibody Response, 31(3-4) IMMUNOL. INVEST. 247-262 (2002), the binding assay described above was modified. Essentially, the reaction with BoNT/A or synthetic BoNT/A peptides was carried out either after absorption of the sera with TeNT or, more conveniently, in the presence of a large excess of TeNT as described further below. The pool of positive control antisera was obtained from human volunteers, and was tested at two dilutions (1:1000 and 1:2000, vol/vol).

Binding studies of the antisera from CD patients as well as sera from unimmunized controls showed that the sera had different levels of non-specific binding to unrelated protein (BSA) and peptides. This high non-specific binding affected both the net cpm values as well as the ratio of the signal (specific binding) to background (non-specific binding). Sera from the same cervical dystonia patients prior to toxin treatment (pre-immune sera) were not available to correct for the non-specific binding. However, the amount of radiolabel bound by certain synthetic H peptides was observed to be essentially the same as the amount of radiolabel bound to unrelated proteins and peptides. These non-antibody-binding H chain peptides (for example, N2, N3, N5, N6, N7, N9, N10, N11, N12, etc.; see FIG. 1) were utilized as an internal control for each serum. In particular, binding was expressed for each serum as a ratio of the amount of antibody bound by a test peptide over the average of the amounts of antibody bound by four of the non-antibody binding H-chain peptides (N2, N12, C17 and C23). The value for such a ratio of antibodies bound to a given peptide from a given serum was essentially constant.

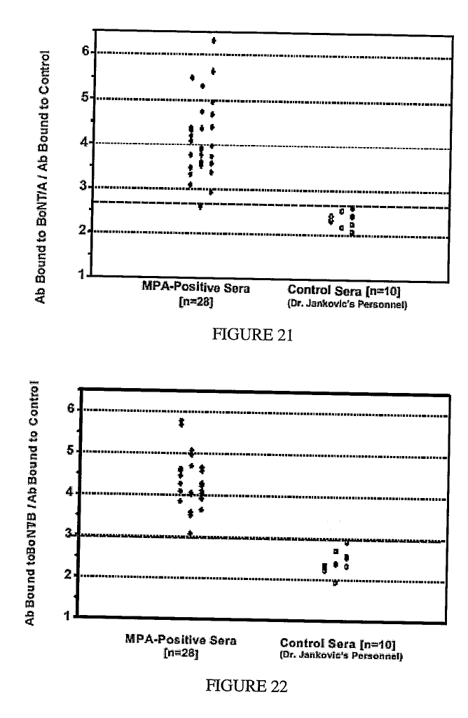

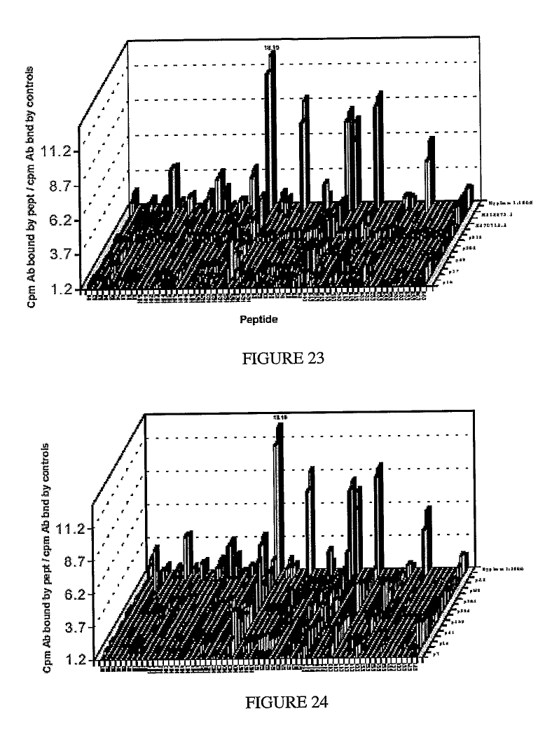

In assays to determine the total amounts of antibody present in CD patient sera, BSA and the four non-binding peptides N2, N12, C17 and C23 were used as negative controls. The results of antibody binding to BoNT/A toxoid in 28 MPA-positive CD sera and 10 human sera from unimmunized controls are summarized in FIG. 21. The results show that 27 out of 28 (96.4%) MPA-positive sera bound antibody levels that were clearly higher than those bound by the controls. These results validate the use of assays performed with human sera in a large excess of TeNT to determine the total amounts of antibodies to BoNT/A present in the serum of a patient in the course of treatment with BOTOX®.

In determining the total amount of anti-BoNT/B antibodies present in CD patient sera, BSA was used as the negative control. The results of binding to the BoNT/B toxoid of antibodies in 28 MPA-positive CD sera and 10 human sera from unimmunized controls are summarized in FIG. 22. The results show that 27 out of 28 (96.4%) of MPA-positive sera bound antibody levels that were clearly higher than those bound by the controls, while one was close to the borderline. These results validate the use of this assay for determining total amounts of antibodies to BoNT/B present in patient serum in the course of treatment with a BoNT/B formulation.

C. Mapping of Epitopes Recognized by Antibodies in of MPA-Positive Aera of Cervical Dystonia Patients

The results of mapping by the synthetic H-chain peptides of antibodies from 28 CD patients that were MPA positive are shown in FIGS. 24 to 26 and summarized in Table 6. These data, which represent four replicate experiments, are compared to binding profiles obtained with hyperimmune human sera at 1:1000 and 1:2000 (vol/vol). As described above, the results in FIGS. 24 to 26 and in Table 6 are based on the ratio of cpm bound by a given peptide/cpm bound by BSA and/or average of cpm bound by peptides N2, N12, C17 and C23. In Table 6, (−) denotes no detectable binding; (±) indicates very low but reproducible binding (ratio of specific over non-specific binding of 1.61-2.0); and different numbers of (+) signs indicate different levels of binding. As can be seen by comparison with the data reported above, peptides which bound antibodies in the sera of the CD patients also bound antibodies within hyperimmune sera. However, not every peptide that bound antibodies in hyperimmune serum was able to bind antibodies in patient sera, indicating that the antibody-binding profile of the patients' sera was more restricted than the profile of the hyperimmune sera.

Furthermore, variability was seen among the binding profiles for different patients. As an example, the antisera of some patients bound peptide N4, whereas other sera had no such binding-activity. This inter-patient variability is consistent with the fact that immune responses to protein antigens are known to be under genetic control and that the response to each epitope within a protein is under separate genetic control, see, e.g., K. Okuda et al., Genetic Control of Immune Response to Sperm Whale Myoglobin in Mice. I. T Lymphocyte Proliferative Response Under H-2-Linked Ir Gene Control, 121(3) J. IMMUNOL. 866-868 (1978).

Significantly, however, some peptides bound antibodies in most of the patients. For example, 25 out of 28 sera contained antibodies that bound to peptide N25, although the amounts bound varied from patient to patient with three sera (patients 45, 304 and 310) having marginal levels of antibodies to this peptide. Peptide C10 bound antibodies in sera of 24 out of 28 patients, with the sera of patients 43, 45, 53 and SD displaying very low (±) or no (−) antibody binding to peptide C10. Peptide C15 displayed low (+) to medium (++) binding to antibodies in 17 patient sera; very low (±) binding with nine patient sera; and no binding with two sera. The antibody-binding activity of peptide C20 was generally lower than peptides N25 or C10 but was low (+) to high (+++) in nine of the patient sera, while eight sera showed no antibody binding, and 11 sera showed very low (±), but reproducible, levels of binding. In addition, peptide C31 bound antibodies in 17 sera, showed very low binding in eight patient sera, and displayed no detectable antibody binding with three patient sera. These results indicate that, while there is some peptide-binding variability among MPA-positive CD patient sera, several synthetic BoNT/A peptides bind antibodies in the large majority of patient sera.

D. Synthetic Peptide Assay for Analysis of Reactivity of MPA-Positive Patient Sera

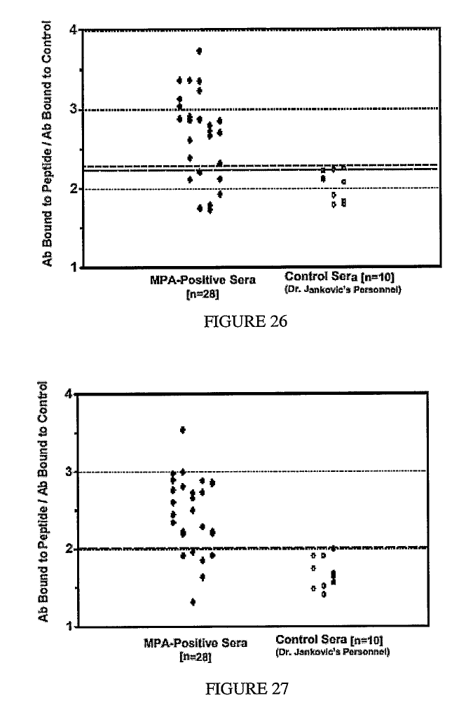

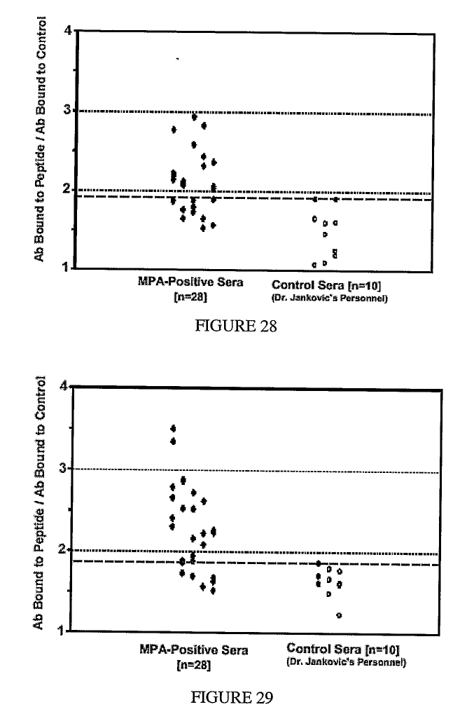

As disclosed above, MPA-positive cervical dystonia patient sera contained antibodies that bound to one or more of the peptides N25, C10, C15, C20 and C31, indicating that binding to one or more of these peptides can used to determine the presence of antibody responses in patient sera. FIG. 27, 28, 29 and 30 show the ratio of the specific cpm bound in the same assay to non-binding peptides and to BSA. As shown in FIG. 26, analysis on the basis of peptide N25 was able to distinguish clearly 21 out of 28 (75%) of patient sera from unimmunized controls. Binding to peptide C10 was also able to distinguish 21 out of 28 sera from the controls, while binding to peptides C15 and C31 distinguished 18 (64.3%) and 20 (71.4%) out of 28 sera, respectively (FIGS. 28 to 30).

Combinations of two or more peptides were also assayed for their discriminatory capability. As shown in FIG. 30, when peptides N25 and C10 were combined in the assay, 25 out of 28 (89.3%) of the CD sera were discriminated from controls. The combination of peptides N25, C10 and C31 distinguished 24 out of 28 sera (85.7%; FIG. 31), and the combination of peptides N25, C10 and C15 distinguished 25 out of 28 (89.3%) of the MPA-positive CD sera from controls (see FIG. 32). Finally, a combination of four peptides (N25, C10, C15 and C31) distinguished 21 out of 28 sera (75%) from the controls, as shown in FIG. 33. These results demonstrate that a combination assay using peptides N25 and C10 or N25, C10 and C15 can be useful for detecting the presence of specific anti-toxin antibodies in BOTOX® treated patients.

Throughout this application various publications have been referenced within parentheses. The disclosures of these publications in their entireties are hereby incorporated by reference in this application in order to more fully describe the state of the art to which this invention pertains.

Although the invention has been described with reference to the disclosed embodiments, those skilled in the art will readily appreciate that the specific experiments detailed are only illustrative of the invention. It should be understood that various modifications can be made without departing from the spirit of the invention.

### Example 10

### Mapping of Synaptosome-Binding Regions of the Heavy Chain of Botulinum Neurotoxin A by Synthetic Overlapping Peptides Encompassing the Entire Chain

A. Binding of 125I-Labled BONT/A to Synaptosomes

To make radioactively-labeled active BoNT/A toxin, active BoNT/A (Metabiologics, Inc., Madison, Wis.) was labeled with ¹²⁵Iodine using a chloramine T method as described in, e.g. W. M Hunter & F. C. Greenwood, Preparation of Iodine-131-labeled human growth hormone of high specific activity, 194 NATURE 495-496, (1962). A labeling reaction comprising 50 μl of 100 mM potassium phosphate, pH 8.0 containing 1.0 μg active BoNT/A toxin, 5 μl of 10 mCi/mL sodium ¹²⁵Iodine, and 25 μl of 100 mM potassium phosphate, pH 8.0 containing 2 mg/mL chloramine T was incubated on ice for 5 minutes. To this labeling reaction, 50 μl of 100 mM potassium phosphate, pH 8.0 containing 20 mg/mL sodium metabisulfite was added to stop the reaction. Excess unlabeled radioactive iodine was removed from the ¹²⁵Iodine-labeled toxin by applying the labeling mixture through a Sephadex G-25 gel filtration column equilibrated and eluted as a single fraction with a column solution comprising 10 mM phosphate-buffered saline, pH 7.2; 150 mM sodium chloride; and 0.1% bovine serum albumin (BSA). The level of ¹²⁵Iodine incorporation was determined by measuring the radioactivity from a 1 μL aliquot using a gamma scintillation counter. The ¹²⁵Iodine-labeled BoNT/A peptide containing eluent was adjusted to a radioactivity level suitable for synaptosome binding assays. The ¹²⁵Iodine-labeled active BoNT/A toxin was stored at 4° C. and used within two days.

To determine the amount of synaptosome required to achieve saturation binding with a fixed amount of BoNT/A peptide, a synaptosome binding assay was conducted. Approximately 50,000 counts/minute of ¹²⁵I-labeled active BoNT/A peptide was mixed with increasing volumes of a synaptosome preparation (from 0 to 8 μL) in 100 μL of Ringer's solution, pH 7.0 (120 mM sodium chloride, 2.5 mM potassium chloride, 2 mM calcium chloride, 4 mM magnesium chloride, 5 mM 2-amino-2-hydroxymethyl-1,3-propanediol hydrochloric acid (Tris-HCl, pH 7.0), 0.5% (w/v) bovine serum albumin). The reaction mixtures were incubated at 37° C. for 20 minutes in order to allow for the formation of any peptide-synaptosome complexes. The reaction mixtures were then microcentrifuged (23,000×g at 20° C. for 3 minutes) to pellet peptide-synaptosome complexes. The pellets were washed twice in 800 μL of Ringer's solution, pH 7.0 to remove any unbound toxin. The pellets containing peptide-synaptosome complexes were resuspended in 300 μL of Ringer's solution, pH 7.0, transferred to a glass scintillation tube, and the amount of radioactivity from these complexes measured using a gamma scintillation counter. The percent synaptosome binding inhibition of a synthetic peptide sample was calculated using the following formula: [1−(count of the sample/count of control)]×100. The experiment was carried out in triplicate.

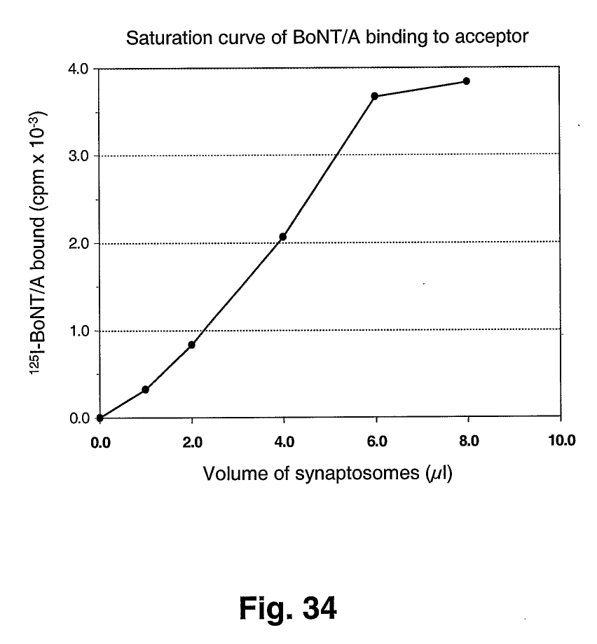

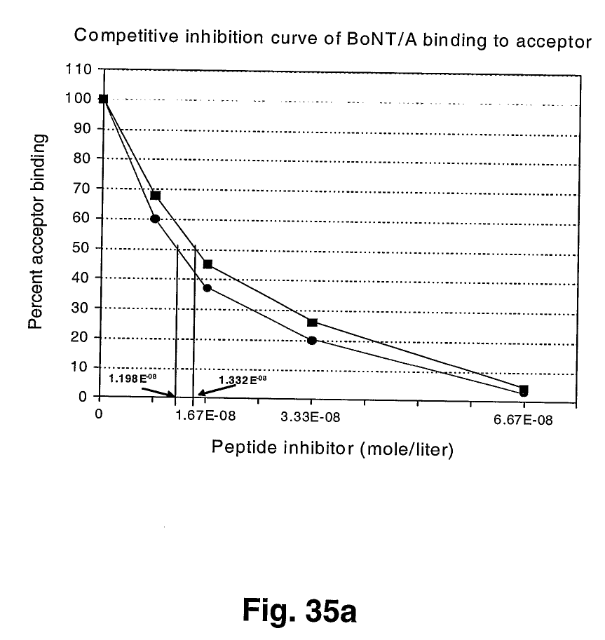

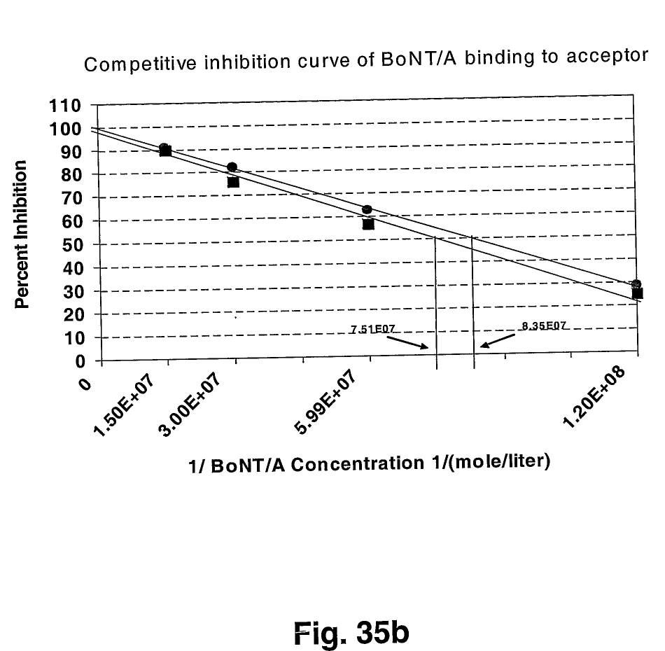

Titration of a fixed amount of ¹²⁵I-labeled BoNT/A (50,000 cpm) with increasing amounts of synaptosomes is shown in FIG. 34. The amount of labeled toxin bound increased until it reached a plateau of about 8% of the added labeled toxin at about 6 μl of synaptosomes. FIG. 35 shows an example of the inhibition of the binding of labeled BoNT/A by unlabeled toxin. It can be seen in FIG. 35a that the binding decreased steadily in the presence of increasing amounts of unlabeled BoNT/A. The binding was completely (100%) inhibited by unlabeled BoNT/A (FIG. 35b), but not by unrelated proteins indicating that the binding of ¹²⁵I-labeled BoNT/A to synaptosomes was entirely specific. The 50% inhibition value (IC₅₀) was obtained at an inhibitor concentration of 1.2×10⁻⁸ M. I also carried out titrations to determine inhibition of the binding of each of active BoNT/A or inactivated toxin (toxoid) to synaptosomes by the other. It was found that toxoid inhibited at maximum (2 μg) the synaptosome-binding of active toxin completely (98%) while maximum inhibition at plateau (2 μg) by active toxin of toxoid synaptosome-binding was 78%.

B. Inhibition by the Individual Peptides of the Binding of 125I-Labled BoNT/A to Synaptosomes

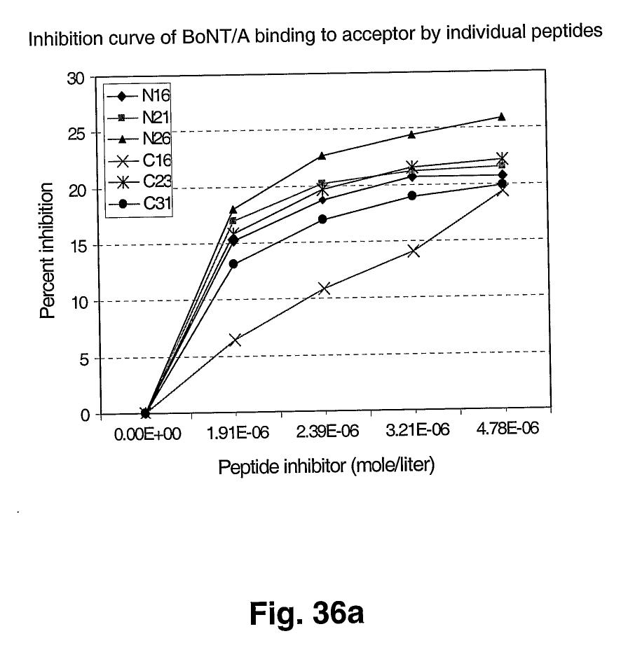

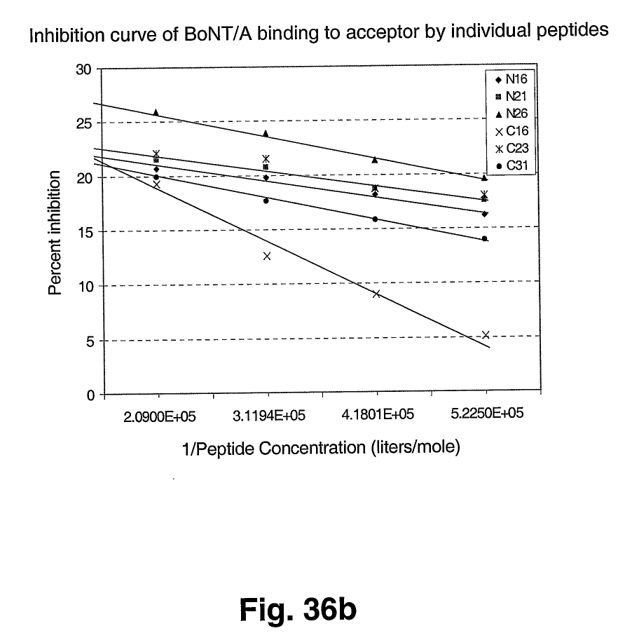

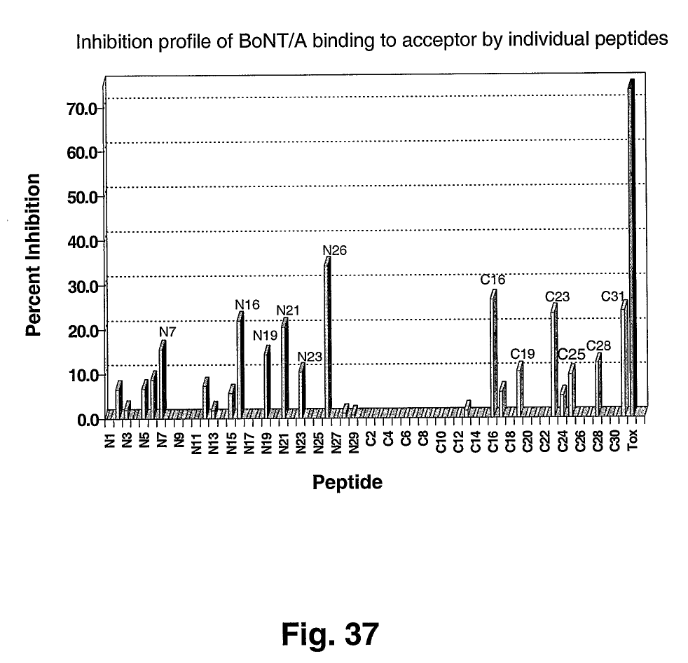

The regions of the H chain involved in the binding to synaptosomes were mapped by determining the ability of each peptide to inhibit the binding of ¹²⁵I-labeled BoNT/A to synaptosomes. FIG. 36a shows an example of the inhibition curves obtained with the synthetic peptides. The values of maximum inhibition were obtained by plotting the inhibition values against the reciprocal of the different peptide concentrations used in the inhibition assay (see FIG. 36b). The maximum inhibitory activities of the 60 peptides are summarized in FIG. 37. The results showed that the synaptosome-binding regions were not only present on the H_{C} domain, but a number of such regions were also found on the H_{N} domain (FIG. 37). On the H_{N} domain, inhibitory activities greater than 10% were exhibited, in decreasing order, by peptides N26 (33.4%), N21 (25.0%), N16 (23.2%), N7 (15.7%), N19 (14.4%), and N23 (10.3%). Five other peptides, N2, N5, N6, N12 and N15, possessed inhibitory activities between 5.6-8.7%. The remaining 18 H_{N} peptides had little or no detectable inhibitory activity. In the H_{C} domain, regions within peptides C16, C23 and C31 had the highest inhibitory activities (between 25-29%), followed in inhibitory activity (10-12%) by peptides C19, C25 and C28. Two other peptides, C17 and C24, had low inhibitory activities (5.8 and 4.9%, respectively). The remaining 23 H_{C} peptides showed little or no detectable inhibitory activity.

C. Inhibition of the Binding of 125I-Labled BoNT/A to Synaptosomes by Mixtures of Equimolar Quantities of the Active Peptides

The competitive inhibitory activities were also determined for mixtures containing equimolar quantities of various H_{C} and H_{N} synthetic peptides. For example, the following peptide mixtures were used as the competitive inhibitor peptide source: (1) The six H_{N} peptides N7, N16, N19, N21, N23 and N26 in a mixture containing 0.167 μg of each peptide in 100 μL of reaction mixture; (2) the five H_{C} peptides C16, C19, C23, C28 and C31 in a mixture containing 0.200 μg of each peptide in 100 μL of reaction mixture; (3) all eleven H_{N}/H_{C peptides N}7, N16, N19, N21, N23, N26, C16, C19, C23, C28 and C31 in a mixture containing 0.091 μg of each peptide. In these experiments the amounts of inhibiting peptide mixture used were increased up to 1 μg/100 μL of each reaction mixture. Under these conditions, the controls that did not have synaptosomes showed no non-specific binding of ¹²⁵I-labeled BoNT/A to the peptides. When higher amounts of peptide mixture were used, some non-specific binding of ¹²⁵I-labeled BoNT/A to the peptides was observed, which increased with the amount of peptide mixture and thus afforded unreliable inhibition values. In addition, the inhibitory capability of each peptide was determined individually FIG. 38 and Table 1 show the inhibitory activities of the three mixtures.

The mixture of the H_{N} peptides contained at maximum amount 0.167 μg of each of the six peptides and exhibited a maximum inhibitory activity of 30.1%. At this excess, the sum of the inhibition of the six peptides is expected to be 31.2%. This compares very well with the inhibition exerted by a mixture containing similar amounts of peptides. The inhibition afforded by the mixture of the five H_{C} peptides (37.4%) was significantly higher than the sum of the inhibition values by the same amount of the individual H_{C} peptides (28.3%). Finally, the inhibition by the mixture of the 11 H_{N} and H_{C} peptides together (44.8%) was also substantially higher than the sum of inhibition values of similar amounts of the individual peptides (31.2%).

TABLE 7
Inhibitory activities of equimolar mixtures of the active peptides

| | Percent inhibition^{a} | |
|---|---|---|
| | Sum of | |
| | individual peptide | Peptide mixture |
| Inhibitor mixture | inhibition (%) | inhibition (%) |
| H_{N} peptides (0.167 μg/peptide) | 31.2 | 30.1 |
| H_{C} peptides (0.200 μg/peptide) | 28.4 | 37.4 |
| H_{N}/H_{C} peptides (0.091 μg/peptide) | 31.2 | 44.8 |
| ^{a}The inhibition of the six H_{N} peptides was determined individually at 0.167 μg or in a mixture containing 0.167 μg of each peptide in 100 μL of reaction mixture; the inhibition of the five H_{C} peptides was determined individually at 0.200 μg or in a mixture containing 0.200 μg of each peptide in 100 μL of reaction mixture; and the inhibition of | | |
| # the six H_{N} peptides and the five H_{C} peptides was determined individually at 0.091 μg or in a mixture containing 0.091 μg of each peptide in 100 μL of reaction mixture. | | |

Tables 8 and 9 compare the peptides that bind protecting mouse anti-BoNT/A Abs (see Tables 1 & 2) to the regions that bind to mouse synaptosomes. Interestingly, many of the synaptosome-binding peptides seem to flank antibody-binding peptides. In the H_{N} domain, the major synaptosome-binding regions within peptides N16, N19, N21 and N23 as well as the minor regions within peptides N2, N12, and N15 did not correspond to binding regions of mouse antibodies. However, the major synaptosome-binding regions within the overlap N6/N7 coincided with an antibody-binding region. The major synaptosome-binding region within peptide N26 shared an overlap with the antibody-binding region within peptide N25 and it is very likely that the two binding regions are displaced relative to another by 2-4 residues. Therefore, antibodies binding to the peptides N6, N7, N8 and to peptide N25 most likely work by blocking the ability of regions N5, N6, N7 and region N26 respectively to bind to synaptosomes. In the H_{C} domain, the major synaptosome-binding regions C16, C17, C19, C23, C24, C25 and C31 also correspond in the vicinity of antibody-binding regions C15, C24 and C31. The extensive correspondence between the synaptosome-binding and the antibody-binding regions on the H_{C} domain can explain the high protective capacity of anti-BoNT/A antibodies (Middlebrook, 1995; Byrne and Smith, 2000; Woodward et al., 2003).

TABLE 8

| | Sequence | | | | | |
|---|---|---|---|---|---|---|
| | Position | | | | | |
| | (Residues of | Antibody Binding^{a} | | | | Synaptosome |
| Peptide No. | SEQ ID NO: 1) | Human | Horse | Mouse | Chicken | Binding^{b} |
| N1 | 449-467 | ++ | − | + | ± | − |
| N2 | 463-481 | − | +++++ | − | − | ++ |
| N3 | 477-495 | − | ± | − | − | − |
| N4 | 491-509 | ++ | + | ± | + | − |
| N5 | 505-523 | − | + | − | − | ++ |
| N6 | 519-537 | ++ | + | +++ | ++ | ++ |
| N7 | 533-551 | ++ | +++ | +++ | +++ | ++++ |
| N8 | 547-565 | +++++ | +++++ | +++++ | +++++ | − |
| N9 | 561-579 | + | ++++ | ++++ | ± | − |
| N10 | 575-593 | ± | ++ | + | ++ | − |
| N11 | 589-607 | +++ | + | − | + | − |
| N12 | 603-621 | + | − | − | − | ++ |
| N13 | 617-635 | − | ± | − | − | − |
| N14 | 631-649 | ++ | ± | ± | + | − |
| N15 | 645-663 | − | − | − | ± | ++ |
| N16 | 659-677 | ++++ | − | − | − | +++++ |
| N17 | 673-691 | ++ | − | ± | ++ | − |
| N18 | 687-705 | + | ± | − | − | − |
| N19 | 701-719 | ± | + | + | ++ | +++ |
| N20 | 715-733 | ++ | ++ | ± | ++ | − |
| N21 | 729-747 | ± | − | − | − | +++++ |
| N22 | 743-761 | ++++ | ++ | + | ++++ | − |
| N23 | 757-775 | − | + | − | − | +++ |
| N24 | 771-789 | ++ | ± | + | + | − |
| N25 | 785-803 | +++++ | +++ | +++++ | +++++ | − |
| N26 | 799-817 | − | − | − | − | +++++ |
| N27 | 813-831 | ++ | ++++ | +++ | ++++ | − |
| N28 | 827-845 | ++ | + | +++ | +++ | − |
| N29 | 841-859 | + | + | − | − | − |
| L-Peptide | 218-231 | − | − | − | − | − |
| Active BoNT/A | — | +++++ | +++++ | +++++ | +++++ | +++++ |

^{a}(+) or (−) signs are based on net cpm values and denote the following: (−), less than 1,500 cpm; (±), 1,500-3,000 cpm; (+), 3,000-7,000 cpm; (++), 7,000-15,000 cpm; (+++), 15,000-25,000 cpm; (++++), 25,000-35,000 cpm; (+++++), exceeding 35,000 cpm.
^{b}(+) or (−) signs are based on percent binding inhibition values of BoNT/A to a synaptosome preperation by a synthetic peptide and denote the following: (−), less than 2% inhibition; (+), 2-5% inhibition; (++), 5-10% inhibition; (+++), 10-15% inhibition; (++++), 15-20% inhibition; (+++++), greater than 20% inhibition.

TABLE 9

| | Sequence | | | | | |
|---|---|---|---|---|---|---|
| | Position | | | | | |
| | (Residues of | Antibody Binding^{a} | | | | Synaptosome |
| Peptide No. | SEQ ID NO: 1) | Human | Horse | Mouse | Chicken | Binding^{b} |
| C1 | 855-873 | − | +++ | ± | − | − |
| C2 | 869-887 | +++ | − | +++ | +++ | − |
| C3 | 883-901 | − | + | + | +++++ | − |
| C4 | 897-915 | − | ± | − | ± | − |
| C5 | 911-929 | ++ | + | − | + | − |
| C6 | 925-943 | +++ | − | − | ++ | − |
| C7 | 939-957 | + | ++ | + | +++++ | − |
| C8 | 953-971 | − | − | − | − | − |
| C9 | 967-985 | + | − | ± | ++++ | − |
| C10 | 981-999 | +++ | ± | − | +++++ | − |
| C11 | 995-1013 | +++++ | + | + | +++++ | − |
| C12 | 1009-1027 | − | − | − | + | − |
| C13 | 1023-1041 | − | + | − | − | − |
| C14 | 1037-1055 | − | + | − | + | − |
| C15 | 1051-1069 | +++++ | ± | ++ | +++++ | − |
| C16 | 1065-1083 | − | − | − | − | +++++ |
| C17 | 1079-1097 | − | + | − | − | ++ |
| C18 | 1093-1111 | − | + | + | ++ | − |
| C19 | 1107-1125 | ± | − | − | − | +++ |
| C20 | 1121-1139 | + | + | ± | +++++ | − |
| C21 | 1135-1153 | ++ | ± | ± | +++ | − |
| C22 | 1149-1167 | ± | + | − | ++ | − |
| C23 | 1163-1181 | ± | − | − | − | +++++ |
| C24 | 1177-1195 | +++ | − | ++ | +++++ | + |
| C25 | 1191-1209 | ± | ++ | − | − | +++ |
| C26 | 1205-1223 | − | + | − | − | − |
| C27 | 1219-1237 | + | − | − | − | − |
| C28 | 1233-1251 | + | − | − | − | +++ |
| C29 | 1247-1265 | ++ | ± | − | ± | − |
| C30 | 1261-1279 | + | ++ | − | +++ | − |
| C31 | 1275-1296 | ++ | +++ | ++ | +++ | +++++ |
| L-Peptide | 218-231 | − | − | − | − | − |
| Active BoNT/A | — | +++++ | +++++ | +++++ | +++++ | +++++ |

^{a}(+) or (−) signs are based on net cpm values and denote the following: (−), less than 1,500 cpm; (±), 1,500-3,000 cpm; (+), 3,000-7,000 cpm; (++), 7,000-15,000 cpm; (+++), 15,000-25,000 cpm; (++++), 25,000-35,000 cpm; (+++++), exceeding 35,000 cpm.
^{b}(+) or (−) signs are based on percent binding inhibition values of BoNT/A to a synaptosome preperation by a synthetic peptide and denote the following: (−), less than 2% inhibition; (+), 2-5% inhibition; (++), 5-10% inhibition; (+++), 10-15% inhibition; (++++), 15-20% inhibition; (+++++), greater than 20% inhibition.

What is claimed:

1. A BoNT/A peptide composition having a length of at most 60 amino acids and wherein said peptide comprises at least 5 contiguous amino acids selected from the group consisting of 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1079-1097 of SEQ ID NO:1 (C17), 1107-1125 of SEQ ID NO:1 (C19), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28) or 1275-1296 of SEQ ID NO:1 (C31).

2. The composition according to claim 1, wherein said amino acid sequence is selected from the group consisting of 729-747 of SEQ ID NO:1 (N21) 799-817 of SEQ ID NO:1 (N26), 1065-1083 of SEQ ID NO:1 (C16), 1163-1181 of SEQ ID NO:1 (C23) or 1275-1296 of SEQ ID NO:1 (C31).

3. The composition according to claim 1, wherein said amino acid sequence is selected from the group consisting of 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1079-1097 of SEQ ID NO:1 (C17), 1107-1125 of SEQ ID NO:1 (C19), 1163-1181 of SEQ ID NO:1 (C23), 1177-1195 of SEQ ID NO:l (C24), 1191-1209 of SEQ ID NO:1 (C25), 1233-1251 of SEQ ID NO:1 (C28) or 1275-1296 of SEQ ID NO:1 (C31), or a conservative variant thereof.

4. The composition according to claim 1, wherein said amino acid sequence is selected from the group consisting of 729-747 of SEQ ID NO:1 (N21) 799-817 of SEQ ID NO:1 (N26), 1065-1083 of SEQ ID NO:1 (C16), 1163-1181 of SEQ ID NO:1 (C23) or 1275-1296 of SEQ ID NO:1 (C31), or a conservative variant thereof.

5. The composition according to claim 1, wherein said amino acid sequence is selected from the group consisting of 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1079-1097 of SEQ ID NO:1 (C17), 1107-1125 of SEQ ID NO:1 (C19), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28) or 1275-1296 of SEQ ID NO:1 (C31), or a non-conservative variant thereof.

6. The composition according to claim 1, wherein said amino acid sequence is selected from the group consisting of 729-747 of SEQ ID NO:1 (N21) 799-817 of SEQ ID NO:1 (N26), 1065-1083 of SEQ ID NO:1 (C16), 1163-1181 of SEQ ID NO:1 (C23) or 1275-1296 of SEQ ID NO:1 (C31), or a non-conservative variant thereof.

7. The composition according to claim 1, wherein said amino acid sequence is selected from the group consisting of 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1079-1097 of SEQ ID NO:1 (C17), 1107-1125 of SEQ ID NO:1 (C19), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28) or 1275-1296 of SEQ ID NO:1 (C31), or an immunoreactive fragment thereof, with the proviso that the BoNT/A peptide is not SEQ ID NO:2.

8. The composition according to claim 1, wherein said amino acid sequence is selected from the group consisting of 729-747 of SEQ ID NO:1 (N21) 799-817 of SEQ ID NO:1 (N26), 1065-1083 of SEQ ID NO:1 (C16), 1163-1181 of SEQ ID NO:1 (C23) or 1275-1296 of SEQ ID NO:1 (C31), or an immunoreactive fragment thereof, with the proviso that the BoNT/A peptide is not SEQ ID NO:2.

9. The composition of claim 1, wherein said BoNT/A peptide has a length of at most 40 amino acids.

10. The composition of claim 1, wherein said BoNT/A peptide has a length of at most 20 amino acids.

11. A tolerogizing composition comprising a tolerogizing agent operationally linked to a BoNT/A peptide having a length of at most 60 amino acids and wherein said peptide comprises at least 5 contiguous amino acids selected from the group consisting of 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1079-1097 of SEQ ID NO:1 (C17), 1107-1125 of SEQ ID NO:1 (C19), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28) or 1275-1296 of SEQ ID NO:1 (C31).

12. The composition according to claim 11, wherein said amino acid sequence is selected from the group consisting of 729-747 of SEQ ID NO:1 (N21) 799-817 of SEQ ID NO:1 (N26), 1065-1083 of SEQ ID NO:1 (C16), 1163-1181 of SEQ ID NO:1 (C23) or 1275-1296 of SEQ ID NO:1 (C31).

13. The composition according to claim 11, wherein said amino acid sequence is selected from the group consisting of 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1079-1097 of SEQ ID NO:1 (C17), 1107-1125 of SEQ ID NO:1 (C19), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28) or 1275-1296 of SEQ ID NO:1 (C31), or a conservative variant thereof.

14. The composition according to claim 11, wherein said amino acid sequence is selected from the group consisting of 729-747 of SEQ ID NO:1 (N21) 799-817 of SEQ ID NO:1 (N26), 1065-1083 of SEQ ID NO:1 (C16), 1163-1181 of SEQ ID NO:1 (C23) or 1275-1296 of SEQ ID NO:1 (C31), or a conservative variant thereof.

15. The composition according to claim 11, wherein said amino acid sequence is selected from the group consisting of 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1079-1097 of SEQ ID NO:1 (C17), 1107-1125 of SEQ ID NO:1 (C19), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28) or 1275-1296 of SEQ ID NO:1 (C31), or a non-conservative variant thereof.

16. The composition according to claim 11, wherein said amino acid sequence is selected from the group consisting of 729-747 of SEQ ID NO:1 (N21) 799-817 of SEQ ID NO:1 (N26), 1065-1083 of SEQ ID NO:1 (C16), 1163-1181 of SEQ ID NO:1 (C23) or 1275-1296 of SEQ ID NO:1 (C31), or a non-conservative variant thereof.

17. The composition according to claim 11, wherein said amino acid sequence is selected from the group consisting of 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1079-1097 of SEQ ID NO:1 (C17), 1107-1125 of SEQ ID NO:1 (C19), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28) or 1275-1296 of SEQ ID NO:1 (C31), or an immunoreactive fragment thereof, with the proviso that the BoNT/A peptide is not SEQ ID NO:2.

18. The composition according to claim 11, wherein said amino acid sequence is selected from the group consisting of 729-747 of SEQ ID NO:1 (N21) 799-817 of SEQ ID NO:1 (N26), 1065-1083 of SEQ ID NO:1 (C16), 1163-1181 of SEQ ID NO:1 (C23) or 1275-1296 of SEQ ID NO:1 (C31), or an immunoreactive fragment thereof, with the proviso that the BoNT/A peptide is not SEQ ID NO:2.

19. The composition according to claim 11, wherein said BoNT/A peptide has a length of at most 40 amino acids.

20. The composition according to claim 11, wherein said BoNT/A peptide has a length of at most 20 amino acids.

21. A vaccine composition comprising a BoNT/A peptide having a length of at most 60 amino acids and wherein said peptide comprises at least 5 contiguous amino acids selected from the group consisting of 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1079-1097 of SEQ ID NO:1 (C17), 1107-1125 of SEQ ID NO:1 (C19), 1163-1181 of SEQ ID NO:1 (C23),1177-1195 of SEQ ID NO:1 (C24), 1191-1209 of SEQ ID NO:1 (C25), 1233-1251 of SEQ ID NO:1 (C28) or 1275-1296 of SEQ ID NO:1 (C31).

22. The composition according to claim 21, wherein said amino acid sequence is selected from the group consisting of 729-747 of SEQ ID NO:1 (N21) 799-817 of SEQ ID NO:1 (N26), 1065-1083 of SEQ ID NO:l (C16), 1163-1181 of SEQ ID NO:l (C23) or 1275-1296 of SEQ ID NO:1 (C31).

23. The composition according to claim 21, wherein said amino acid sequence is selected from the group consisting of 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1079-1097 of SEQ ID NO:1 (C17), 1107-1125 of SEQ ID NO:1 (C19), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28) or 1275-1296 of SEQ ID NO:1 (C31), or a conservative variant thereof.

24. The composition according to claim 21, wherein said amino acid sequence is selected from the group consisting of 729-747 of SEQ ID NO:1 (N21) 799-817 of SEQ ID NO:1 (N26), 1065-1083 of SEQ ID NO:1 (C16), 1163-1181 of SEQ ID NO:1 (C23) or 1275-1296 of SEQ ID NO:1 (C31), or a conservative variant thereof.

25. The composition according to claim 21, wherein said amino acid sequence is selected from the group consisting of 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1079-1097 of SEQ ID NO:1 (C17), 1107-1125 of SEQ ID NO:1 (C19), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28) or 1275-1296 of SEQ ID NO:1 (C31), or a non-conservative variant thereof.

26. The composition according to claim 21, wherein said amino acid sequence is selected from the group consisting of 729-747 of SEQ ID NO:1 (N21) 799-817 of SEQ ID NO:1 (N26), 1065-1083 of SEQ ID NO:1 (C16), 1163-1181 of SEQ ID NO:1 (C23) or 1275-1296 of SEQ ID NO:1 (C31), or a non-conservative variant thereof.

27. The composition according to claim 21, wherein said amino acid sequence is selected from the group consisting of 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1079-1097 of SEQ ID NO:1 (C17), 1107-1125 of SEQ ID NO:1 (C19), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28) or 1275-1296 of SEQ ID NO:1 (C31), or an immunoreactive fragment thereof, with the proviso that the BoNT/A peptide is not SEQ ID NO:2.

28. The composition according to claim 21, wherein said amino acid sequence is selected from the group consisting of 729-747 of SEQ ID NO:1 (N21) 799-817 of SEQ ID NO:1 (N26), 1065-1083 of SEQ ID NO:1 (C16), 1163-1181 of SEQ ID NO:1 (C23) or 1275-1296 of SEQ ID NO:1 (C31), or an immunoreactive fragment thereof, with the proviso that the BoNT/A peptide is not SEQ ID NO:2.

29. The composition according to claim 21, wherein said BoNT/A peptide has a length of at most 40 amino acids.

30. The composition according to claim 21, wherein said BoNT/A peptide has a length of at most 20 amino acids.

31. An antibody composition prepared from a BoNT/A peptide having a length of at most 60 amino acids and wherein said peptide comprises at least 5 contiguous amino acids selected from the group consisting of 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1079-1097 of SEQ ID NO:1 (C17), 1107-1125 of SEQ ID NO:l (C19), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28) or 1275-1296 of SEQ ID NO:1 (C31).

32. The composition according to claim 31, wherein said amino acid sequence is selected from the group consisting of 729-747 of SEQ ID NO:1 (N21) 799-817 of SEQ ID NO:1 (N26), 1065-1083 of SEQ ID NO:1 (C16), 1163-1181 of SEQ ID NO:1 (C23) or 1275-1296 of SEQ ID NO:1 (C31).

33. The composition according to claim 31, wherein said amino acid sequence is selected from the group consisting of 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1079-1097 of SEQ ID NO:1 (C17), 1107-1125 of SEQ ID NO:1 (C19), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28) or 1275-1296 of SEQ ID NO:1 (C31), or a conservative variant thereof.

34. The composition according to claim 31, wherein said amino acid sequence is selected from the group consisting of 729-747 of SEQ ID NO:1 (N21) 799-817 of SEQ ID NO:1 (N26), 1065-1083 of SEQ ID NO:1 (C16), 1163-1181 of SEQ ID NO:1 (C23) or 1275-1296 of SEQ ID NO:1 (C31), or a conservative variant thereof.

35. The composition according to claim 31, wherein said amino acid sequence is selected from the group consisting of 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1079-1097 of SEQ ID NO:1 (C17), 1107-1125 of SEQ ID NO:1 (C19), 1163-1181 of SEQ ID NO:1 (C23),1177-1195 of SEQ ID NO:1 (C24), 1191-1209 of SEQ ID NO:1 (C25), 1233-1251 of SEQ ID NO:1 (C28) or 1275-1296 of SEQ ID NO:1 (C31), or a non-conservative variant thereof.

36. The composition according to claim 31, wherein said amino acid sequence is selected from the group consisting of 729-747 of SEQ ID NO:1 (N21) 799-817 of SEQ ID NO:1 (N26), 1065-1083 of SEQ ID NO:1 (C16), 1163-1181 of SEQ ID NO:1 (C23) or 1275-1296 of SEQ ID NO:1 (C31), or a non-conservative variant thereof.

37. The composition according to claim 31, wherein said amino acid sequence is selected from the group consisting of 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1079-1097 of SEQ ID NO:1 (C17), 1107-1125 of SEQ ID NO:1 (C19), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28) or 1275-1296 of SEQ ID NO:1 (C31), or an immunoreactive fragment thereof, with the proviso that the BoNT/A peptide is not SEQ ID NO:2.

38. The composition according to claim 31, wherein said amino acid sequence is selected from the group consisting of 729-747 of SEQ ID NO:1 (N21) 799-817 of SEQ ID NO:1 (N26), 1065-1083 of SEQ ID NO:l (C16), 1163-1181 of SEQ ID NO:1 (C23) or 1275-1296 of SEQ ID NO:1 (C31), or an immunoreactive fragment thereof, with the proviso that the BoNT/A peptide is not SEQ ID NO:2.

39. The composition according to claim 31, wherein said BoNT/A peptide has a length of at most 40 amino acids.

40. The composition according to claim 31, wherein said BoNT/A peptide has a length of at most 20 amino acids.

41. A method of determining immunoresistance to botulinum toxin therapy in a human or other mammal by determining the presence or absence in said human or other mammal of antibodies immunoreactive with a BoNT/A peptide composition having a length of at most 60 amino acids and wherein said peptide comprises at least 5 contiguous amino acids selected from the group consisting of 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1079-1097 of SEQ ID NO:1 (C17), 1107-1125 of SEQ ID NO:1 (C19), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28) or 1275-1296 of SEQ ID NO:1 (C31), where the presence of said antibodies immunoreactive with the said BoNT/A peptide indicates immunoresistance to said therapy.

42. The method according to claim 41, wherein said amino acid sequence is selected from the group consisting of 729-747 of SEQ ID NO:1 (N21) 799-817 of SEQ ID NO:1 (N26), 1065-1083 of SEQ ID NO:l (C16), 1163-1181 of SEQ ID NO:1 (C23) or 1275-1296 of SEQ ID NO:1 (C31).

43. The method according to claim 41, wherein said amino acid sequence is selected from the group consisting of 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1079-1097 of SEQ ID NO:1 (C17), 1107-1125 of SEQ ID NO:1 (C19), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28) or 1275-1296 of SEQ ID NO:1 (C31), or a conservative variant thereof.

44. The method according to claim 41, wherein said amino acid sequence is selected from the group consisting of 729-747 of SEQ ID NO:1 (N21) 799-817 of SEQ ID NO:1 (N26), 1065-1083 of SEQ ID NO:1 (C16), 1163-1181 of SEQ ID NO:1 (C23) or 1275-1296 of SEQ ID NO:1 (C31), or a conservative variant thereof.

45. The method according to claim 41, wherein said amino acid sequence is selected from the group consisting of 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1079-1097 of SEQ ID NO:1 (C17), 1107-1125 of SEQ ID NO:1 (C19), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28) or 1275-1296 of SEQ ID NO:1 (C31), or a non-conservative variant thereof.

46. The method according to claim 41, wherein said amino acid sequence is selected from the group consisting of 729-747 of SEQ ID NO:1 (N21) 799-817 of SEQ ID NO:1 (N26), 1065-1083 of SEQ ID NO:1 (C16), 1163-1181 of SEQ ID NO:1 (C23) or 1275-1296 of SEQ ID NO:1 (C31), or a non-conservative variant thereof.

47. The method according to claim 41, wherein said amino acid sequence is selected from the group consisting of 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1079-1097 of SEQ ID NO:1 (C17), 1107-1125 of SEQ ID NO:1 (C19), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28) or 1275-1296 of SEQ ID NO:1 (C31), or an immunoreactive fragment thereof, with the proviso that the BoNT/A peptide is not SEQ ID NO:2.

48. The method according to claim 41, wherein said amino acid sequence is selected from the group consisting of 729-747 of SEQ ID NO:1 (N21) 799-817 of SEQ ID NO:1 (N26), 1065-1083 of SEQ ID NO:1 (C16), 1163-1181 of SEQ ID NO:1 (C23) or 1275-1296 of SEQ ID NO:1 (C31), or an immunoreactive fragment thereof, with the proviso that the BoNT/A peptide is not SEQ ID NO:2.

49. The method according to claim 41, wherein said BoNT/A peptide has a length of at most 40 amino acids.

50. The method according to claim 41, wherein said BoNT/A peptide has a length of at most 20 amino acids.

51. The method according to claim 41, wherein said peptide is immobilized on a solid support.

52. The method according to claim 41, wherein said presence or absence of antibodies immunoreactive with said BoNT/A peptide is determined using an enzyme-linked immunosorbent assay.

53. The method according to claim 41, wherein said presence or absence of antibodies immunoreactive with said BoNT/A peptide is determined using a radioimmunoassay.

54. The method according to claim 41, wherein said presence or absence of antibodies immunoreactive with said BoNT/A peptide composition is determined by selectively determining the presence or absence of IgG antibodies immunoreactive with said BoNT/A peptide.

55. The method according to claim 41, wherein said botulinum toxin therapy is a BoNT/A therapy.

56. A method of preventing or reducing immunoresistance to botulinum toxin therapy in a human or other mammal by administering to said human or other mammal a tolerogizing composition comprising a tolerogizing agent operationally linked to a BoNT/A peptide having a length of at most 60 amino acids and wherein said peptide comprises at least 5 contiguous amino acids selected from the group consisting of 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1079-1097 of SEQ ID NO:1 (C17), 1107-1125 of SEQ ID NO:1 (C19), 1163-1181 of SEQ ID NO:1 (C23), 1177-1195 of SEQ ID NO:1 (C24),1191-1209 of SEQ ID NO:1 (C25), 1233-1251 of SEQ ID NO:1 (C28) or 1275-1296 of SEQ ID NO:1 (C31), or a conservative variant, a non-conservative variant or an immunoreactive fragment thereof, with the proviso that the BoNT/A peptide is not SEQ ID NO:2.

57. The method according to claim 56, wherein said amino acid sequence is selected from the group consisting of 729-747 of SEQ ID NO:1 (N21) 799-817 of SEQ ID NO:1 (N26), 1065-1083 of SEQ ID NO:1 (C16), 1163-1181 of SEQ ID NO:1 (C23) or 1275-1296 of SEQ ID NO:1 (C31).

58. The method according to claim 56, wherein said amino acid sequence is selected from the group consisting of 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1079-1097 of SEQ ID NO:1 (C17), 1107-1125 of SEQ ID NO:1 (C19), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28) or 1275-1296 of SEQ ID NO:1 (C31), or a conservative variant thereof.

59. The method according to claim 56, wherein said amino acid sequence is selected from the group consisting of 729-747 of SEQ ID NO:1 (N21) 799-817 of SEQ ID NO:1 (N26), 1065-1083 of SEQ ID NO:1 (C16), 1163-1181 of SEQ ID NO:1 (C23) or 1275-1296 of SEQ ID NO:1 (C31), or a conservative variant thereof.

60. The method according to claim 56, wherein said amino acid sequence is selected from the group consisting of 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1079-1097 of SEQ ID NO:1 (C17), 1107-1125 of SEQ ID NO:1 (C19), 1163-1181 of SEQ ID NO:1 (C23), 1177-1195 of SEQ ID NO:l (C24), 1191-1209 of SEQ ID NO:1 (C25), 1233-1251 of SEQ ID NO:1 (C28) or 1275-1296 of SEQ ID NO:1 (C31), or a non-conservative variant thereof.

61. The method according to claim 56, wherein said amino acid sequence is selected from the group consisting of 729-747 of SEQ ID NO:1 (N21) 799-817 of SEQ ID NO:1 (N26), 1065-1083 of SEQ ID NO:1 (C16), 1163-1181 of SEQ ID NO:1 (C23) or 1275-1296 of SEQ ID NO:1 (C31), or a non-conservative variant thereof.

62. The method according to claim 56, wherein said amino acid sequence is selected from the group consisting of 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1079-1097 of SEQ ID NO:1 (C17), 1107-1125 of SEQ ID NO:1 (C19), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28) or 1275-1296 of SEQ ID NO:1 (C31), or an immunoreactive fragment thereof, with the proviso that the BoNT/A peptide is not SEQ ID NO:2.

63. The method according to claim 56, wherein said amino acid sequence is selected from the group consisting of 729-747 of SEQ ID NO:1 (N21) 799-817 of SEQ ID NO:1 (N26), 1065-1083 of SEQ ID NO:1 (C16), 1163-1181 of SEQ ID NO:1 (C23) or 1275-1296 of SEQ ID NO:1 (C31), or an immunoreactive fragment thereof, with the proviso that the BoNT/A peptide is not SEQ ID NO:2.

64. The method according to claim 56, wherein said BoNT/A peptide has a length of at most 40 amino acids.

65. The method according to claim 56, wherein said BoNT/A peptide has a length of at most 20 amino acids.

66. The method according to claim 56, wherein said botulinum toxin therapy is a BoNT/A therapy.

67. The method according to claim 56, wherein said botulinum toxin therapy is a BoNT/E therapy.

68. A method of preventing or reducing BoNT/A toxicity in a human or other mammal by administering to a human or other mammal a vaccine composition comprising a BoNT/A peptide having a length of at most 60 amino acids and wherein said peptide comprises at least 5 contiguous amino acids selected from the group consisting of 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1079-1097 of SEQ ID NO:1 (C17), 1107-1125 of SEQ ID NO:1 (C19), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28) or 1275-1296 of SEQ ID NO:1 (C31), thereby producing an immune response to botulinum toxin in said human or other mammal, with the proviso that the BoNT/A peptide is not SEQ ID NO:2.

69. The method according to claim 68, wherein said amino acid sequence is selected from the group consisting of 729-747 of SEQ ID NO:1 (N21) 799-817 of SEQ ID NO:1 (N26), 1065-1083 of SEQ ID NO:1 (C16), 1163-1181 of SEQ ID NO:1 (C23) or 1275-1296 of SEQ ID NO:1 (C31).

70. The method according to claim 68, wherein said amino acid sequence is selected from the group consisting of 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1079-1097 of SEQ ID NO:1 (C17), 1107-1125 of SEQ ID NO:1 (C19), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28) or 1275-1296 of SEQ ID NO:1 (C31), or a conservative variant thereof.

71. The method according to claim 68, wherein said amino acid sequence is selected from the group consisting of 729-747 of SEQ ID NO:1 (N21) 799-817 of SEQ ID NO:1 (N26), 1065-1083 of SEQ ID NO:1 (C16), 1163-1181 of SEQ ID NO:1 (C23) or 1275-1296 of SEQ ID NO:1 (C31), or a conservative variant thereof.

72. The method according to claim 68, wherein said amino acid sequence is selected from the group consisting of 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1079-1097 of SEQ ID NO:1 (C17), 1107-1125 of SEQ ID NO:1 (C19), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28) or 1275-1296 of SEQ ID NO:1 (C31), or a non-conservative variant thereof.

73. The method according to claim 68, wherein said amino acid sequence is selected from the group consisting of 729-747 of SEQ ID NO:1 (N21) 799-817 of SEQ ID NO:1 (N26), 1065-1083 of SEQ ID NO:1 (C16), 1163-1181 of SEQ ID NO:1 (C23) or 1275-1296 of SEQ ID NO:1 (C31), or a non-conservative variant thereof.

74. The method according to claim 68, wherein said amino acid sequence is selected from the group consisting of 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1079-1097 of SEQ ID NO:1 (C17), 1107-1125 of SEQ ID NO:1 (C19), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28) or 1275-1296 of SEQ ID NO:1 (C31), or an immunoreactive fragment thereof, with the proviso that the BoNT/A peptide is not SEQ ID NO:2.

75. The method according to claim 68, wherein said amino acid sequence is selected from the group consisting of 729-747 of SEQ ID NO:1 (N21) 799-817 of SEQ ID NO:1 (N26), 1065-1083 of SEQ ID NO:1 (C16), 1163-1181 of SEQ ID NO:1 (C23) or 1275-1296 of SEQ ID NO:1 (C31), or an immunoreactive fragment thereof, with the proviso that the BoNT/A peptide is not SEQ ID NO:2.

76. The method according to claim 68, wherein said BoNT/A peptide has a length of at most 40 amino acids.

77. The method according to claim 68, wherein said BoNT/A peptide has a length of at most 20 amino acids.

78. A method of preparing an anti-BoNT/A antibody, comprising the steps of:
a. administering to a human or other mammal a BoNT/A peptide having a length of at most 60 amino acids and wherein said peptide comprises at least 5 contiguous amino acids selected from the group consisting of 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1079-1097 of SEQ ID NO:1 (C17), 1107-1125 of SEQ ID NO:1 (C19), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28) or 1275-1296 of SEQ ID NO:1 (C31);
b. collecting from the animal a sample containing an antibody or antibody-producing cell;
and
c. processing the sample to isolate the anti-BoNT/A antibody, with the proviso that the BoNT/A peptide is not SEQ ID NO:2.

79. The method according to claim 78, wherein said amino acid sequence is selected from the group consisting of 729-747 of SEQ ID NO:1 (N21) 799-817 of SEQ ID NO:1 (N26), 1065-1083 of SEQ ID NO:1 (C16), 1163-1181 of SEQ ID NO:1 (C23) or 1275-1296 of SEQ ID NO:1 (C31).

80. The method according to claim 78, wherein said amino acid sequence is selected from the group consisting of 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1079-1097 of SEQ ID NO:1 (C17), 1107-1125 of SEQ ID NO:1 (C19), 1163-1181 SEQ ID NO:1 (C23), 1177-1195 of SEQ ID NO:1 (C24), 1191-1209 of SEQ ID NO:1 (C25), 1233-1251 of SEQ ID NO:1 (C28) or 1275-1296 of SEQ ID NO:1 (C31), or a conservative variant thereof.

81. The method according to claim 78, wherein said amino acid sequence is selected from the group consisting of 729-747 of SEQ ID NO:1 (N21) 799-817 of SEQ ID NO:1 (N26), 1065-1083 of SEQ ID NO:1 (C16), 1163-1181 of SEQ ID NO:1 (C23) or 1275-1296 of SEQ ID NO:1 (C31), or a conservative variant thereof.

82. The method according to claim 78, wherein said amino acid sequence is selected from the group consisting of 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1079-1097 of SEQ ID NO:1 (C17), 1107-1125 of SEQ ID NO:l (C19), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28) or 1275-1296 of SEQ ID NO:1 (C31), or a non-conservative variant thereof.

83. The method according to claim 78, wherein said amino acid sequence is selected from the group consisting of 729-747 of SEQ ID NO:1 (N21) 799-817 of SEQ ID NO:1 (N26), 1065-1083 of SEQ ID NO:1 (C16), 1163-1181 of SEQ ID NO:1 (C23) or 1275-1296 of SEQ ID NO:1 (C31), or a non-conservative variant thereof.

84. The method according to claim 78, wherein said amino acid sequence is selected from the group consisting of 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1079-1097 of SEQ ID NO:1 (C17), 1107-1125 of SEQ ID NO:l (C19), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28) or 1275-1296 of SEQ ID NO:1 (C31), or an immunoreactive fragment thereof, with the proviso that the BoNT/A peptide is not SEQ ID NO:2.

85. The method according to claim 78, wherein said amino acid sequence is selected from the group consisting of 729-747 of SEQ ID NO:1 (N21) 799-817 of SEQ ID NO:1 (N26), 1065-1083 of SEQ ID NO:1 (C16), 1163-1181 of SEQ ID NO:1 (C23) or 1275-1296 of SEQ ID NO:1 (C31), or an immunoreactive fragment thereof, with the proviso that the BoNT/A peptide is not SEQ ID NO:2.

86. The method according to claim 78, wherein said BoNT/A peptide has a length of at most 40 amino acids.

87. The method according to claim 78, wherein said BoNT/A peptide has a length of at most 20 amino acids.

88. The method according to claim 78, wherein said antibody is polyclonal.

89. The method according to claim 78, wherein said antibody is monoclonal.

88. A method of treating botulinum toxicity in a human or other mammal by administering to said human or other mammal a pharmaceutical composition comprising an anti-BoNT/A antibody prepared from a BoNT/A peptide having a length of at most 60 amino acids and wherein said peptide comprises at least 5 contiguous amino acids selected from the group consisting of 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1079-1097 of SEQ ID NO:1 (C17), 1107-1125 of SEQ ID NO:1 (C19), 1163-1181 of SEQ ID NO:1 (C23), 1177-1195 of SEQ ID NO:1 (C24), 1191-1209 of SEQ ID NO:l (C25), 1233-1251 of SEQ ID NO:1 (C28) or 1275-1296 of SEQ ID NO:1 (C31).

89. The method according to claim 88, wherein said amino acid sequence is selected from the group consisting of 729-747 of SEQ ID NO:1 (N21) 799-817 of SEQ ID NO:1 (N26), 1065-1083 of SEQ ID NO:1 (C16), 1163-1181 of SEQ ID NO:1 (C23) or 1275-1296 of SEQ ID NO:1 (C31).

90. The method according to claim 88, wherein said amino acid sequence is selected from the group consisting of 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1079-1097 of SEQ ID NO:1 (C17), 1107-1125 of SEQ ID NO:l (C19), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28) or 1275-1296 of SEQ ID NO:1 (C31), or a conservative variant thereof.

91. The method according to claim 88, wherein said amino acid sequence is selected from the group consisting of 729-747 of SEQ ID NO:1 (N21) 799-817 of SEQ ID NO:1 (N26), 1065-1083 of SEQ ID NO:1 (C16), 1163-1181 of SEQ ID NO:1 (C23) or 1275-1296 of SEQ ID NO:1 (C31), or a conservative variant thereof.

92. The method according to claim 88, wherein said amino acid sequence is selected from the group consisting of 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1079-1097 of SEQ ID NO:1 (C17), 1107-1125 of SEQ ID NO:1 (C19), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28) or 1275-1296 of SEQ ID NO:1 (C31), or a non-conservative variant thereof.

93. The method according to claim 88, wherein said amino acid sequence is selected from the group consisting of 729-747 of SEQ ID NO:1 (N21) 799-817 of SEQ ID NO:1 (N26), 1065-1083 of SEQ ID NO:1 (C16), 1163-1181 of SEQ ID NO:1 (C23) or 1275-1296 of SEQ ID NO:1 (C31), or a non-conservative variant thereof.

94. The method according to claim 88, wherein said amino acid sequence is selected from the group consisting of 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1079-1097 of SEQ ID NO:1 (C17), 1107-1125 of SEQ ID NO:l (C19), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28) or 1275-1296 of SEQ ID NO:1 (C31), or an immunoreactive fragment thereof, with the proviso that the BoNT/A peptide is not SEQ ID NO:2.

95. The method according to claim 88, wherein said amino acid sequence is selected from the group consisting of 729-747 of SEQ ID NO:1 (N21) 799-817 of SEQ ID NO:1 (N26), 1065-1083 of SEQ ID NO:1 (C16), 1163-1181 of SEQ ID NO:1 (C23) or 1275-1296 of SEQ ID NO:1 (C31), or an immunoreactive fragment thereof, with the proviso that the BoNT/A peptide is not SEQ ID NO:2.

96. The method according to claim 88, wherein said BoNT/A peptide has a length of at most 40 amino acids.

97. The method according to claim 88, wherein said BoNT/A peptide has a length of at most 20 amino acids.

98. A method of reducing or eliminating botulinum toxin antibodies from a human or other mammal, comprising the steps of:
a. removing blood from said human or other mammal;
b. contacting the blood, or an antibody-containing component thereof, with a length of at most 60 amino acids and wherein said peptide comprises at least 5 contiguous amino acids selected from the group consisting of 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1079-1097 of SEQ ID NO:1 (C17), 1107-1125 of SEQ ID NO:1 (C19), 1163-1181 of SEQ ID NO:l (C23), 1177-1195 of SEQ ID NO:1 (C24), 1191-1209 of SEQ ID NO:1 (C25), 1233-1251 of SEQ ID NO:1 (C28) or 1275-1296 of SEQ ID NO:1 (C31), under conditions suitable for forming a complex of each of said amino acid sequences and said anti-botulinum toxin antibody; and
c. removing the complex from the blood or antibody-containing component thereof.

99. The method according to claim 98, wherein said amino acid sequence is selected from the group consisting of 729-747 of SEQ ID NO:1 (N21) 799-817 of SEQ ID NO:1 (N26), 1065-1083 of SEQ ID NO:1 (C16), 1163-1181 of SEQ ID NO:1 (C23) or 1275-1296 of SEQ ID NO:1 (C31).

100. The method according to claim 98, wherein said amino acid sequence is selected from the group consisting of 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1079-1097 of SEQ ID NO:1 (C17), 1107-1125 of SEQ ID NO:1 (C19), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28) or 1275-1296 of SEQ ID NO:1 (C31), or a conservative variant thereof.

101. The method according to claim 98, wherein said amino acid sequence is selected from the group consisting of 729-747 of SEQ ID NO:1 (N21) 799-817 of SEQ ID NO:1 (N26), 1065-1083 of SEQ ID NO:1 (C16), 1163-1181 of SEQ ID NO:1 (C23) or 1275-1296 of SEQ ID NO:1 (C31), or a conservative variant thereof.

102. The method according to claim 98, wherein said amino acid sequence is selected from the group consisting of 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1079-1097 of SEQ ID NO:1 (C17), 1107-1125 of SEQ ID NO:l (C19), 1163-1181 of SEQ ID NO:1 (C23), 1177-1195 of SEQ ID NO:1 (C24), 1191-1209 of SEQ ID NO:1 (C25), 1233-1251 of SEQ ID NO:1 (C28) or 1275-1296 of SEQ ID NO:1 (C31), or a non-conservative variant thereof.

103. The method according to claim 98, wherein said amino acid sequence is selected from the group consisting of 729-747 of SEQ ID NO:1 (N21) 799-817 of SEQ ID NO:1 (N26), 1065-1083 of SEQ ID NO:1 (C16), 1163-1181 of SEQ ID NO:1 (C23) or 1275-1296 of SEQ ID NO:1 (C31), or a non-conservative variant thereof.

104. The method according to claim 98, wherein said amino acid sequence is selected from the group consisting of 701-719 of SEQ ID NO:1 (N19), 729-747 of SEQ ID NO:1 (N21), 757-775 of SEQ ID NO:1 (N23), 799-817 of SEQ ID NO:1 (N26), 1065-1083 of SEQ ID NO:1 (C16), 1079-1097 of SEQ ID NO:1 (C17), 1107-1125 of SEQ ID NO:1 (C19), 1163-1181 of SEQ ID NO:1 (C23), 1177-1195 of SEQ ID NO:l (C24), 1191-1209 of SEQ ID NO:1 (C25), 1233-1251 of SEQ ID NO:1 (C28) or 1275-1296 of SEQ ID NO:1 (C31), or an immunoreactive fragment thereof, with the proviso that the BoNT/A peptide is not SEQ ID NO:2.

105. The method according to claim 98, wherein said amino acid sequence is selected from the group consisting of 729-747 of SEQ ID NO:1 (N21) 799-817 of SEQ ID NO:1 (N26), 1065-1083 of SEQ ID NO:1 (C16), 1163-1181 of SEQ ID NO:1 (C23) or 1275-1296 of SEQ ID NO:1 (C31), or an immunoreactive fragment thereof, with the proviso that the BoNT/A peptide is not SEQ ID NO:2.

106. The method according to claim 98, wherein removing said complex comprising selectively removing IgG botulinum toxin blocking antibodies from said patient.

107. The method according to claim 98, wherein said BoNT/A peptide has a length of at most 40 amino acids.

108. The method according to claim 98, wherein said BoNT/A peptide has a length of at most 20 amino acids.
